(19) United States
(12) Patent Application Publication
(10) Pub. No.: US 2021/0167299 A1
(43) Pub. Date: Jun. 3, 2021

(54) HETEROCYCLIC COMPOUND AND ORGANIC LIGHT-EMITTING DEVICE INCLUDING THE SAME

(71) Applicant: Samsung Electronics Co., Ltd., Suwon-si (KR)

(72) Inventors: Yeonsook Chung, Seoul (KR); Yongsik Jung, Seoul (KR); Hosuk Kang, Suwon-si (KR); Sangmo Kim, Suwon-si (KR); Joonghyuk Kim, Seoul (KR); Sungho Nam, Daegu (KR); Jun Chwae, Seoul (KR)

(21) Appl. No.: 17/036,083

(22) Filed: Sep. 29, 2020

(30) Foreign Application Priority Data
Dec. 2, 2019 (KR) 10-2019-0158454

(51) Int. Cl. H01L 51/00; C07D 307/91; C09K 11/06

(52) U.S. Cl. CPC H01L 51/0073; C07D 307/91; H01L 51/5012; H01L 51/0058; C09K 11/06

(57) ABSTRACT

Provided are a heterocyclic compound represented by Formula 1 and having an asymmetric structure and an organic light-emitting device including the heterocyclic compound:

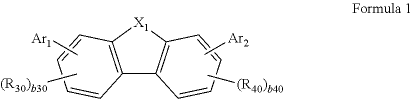

wherein, in Formula 1, X₁ is O or Se, Ar₁ is a group represented by Formula 1A, and Ar₂ is a group represented by Formula 1B,

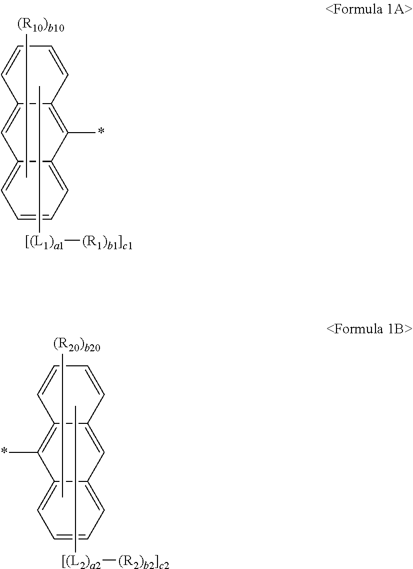

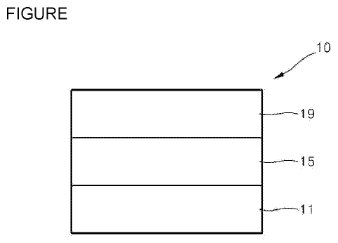

and the other substituents are understood as described in connection with the detailed description.

### CROSS-REFERENCE TO RELATED APPLICATION

This application claims priority to and the benefit of Korean Patent Application No. 10-2019-0158454, filed on Dec. 2, 2019, in the Korean Intellectual Property Office, the content of which is incorporated herein in its entirety by reference.

### BACKGROUND

### 1. Field

One or more embodiments relate to a heterocyclic compound and an organic light-emitting device including the same.

### 2. Description of Related Art

Organic light-emitting devices are self-emission devices, which have improved characteristics in terms of a viewing angle, a response time, brightness, a driving voltage, and a response speed, and produce full-color images.

In an example, an organic light-emitting device includes an anode, a cathode, and an organic layer between the anode and the cathode, wherein the organic layer includes an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transit from an excited state to a ground state, thereby generating light.

### SUMMARY

One or more embodiments provide a heterocyclic compound and an organic light-emitting device including the same.

Additional aspects will be set forth in part in the description which follows and, in part, will be apparent from the description, or may be learned by practice of the presented embodiments of the disclosure.

An aspect of the present disclosure provides a heterocyclic compound represented by Formula 1 and having an asymmetric structure.

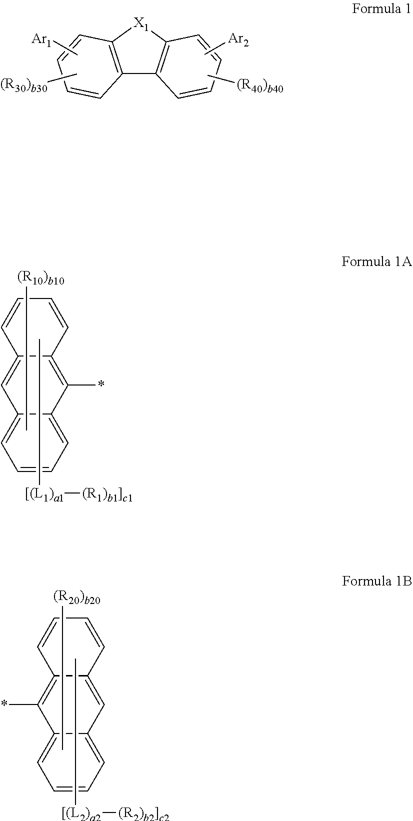

In Formulae 1, 1A, and 1B,

X₁ may be O or Se,

Ar₁ may be a group represented by Formula 1A and Are may be a group represented by Formula 1B,

L₁ and L₂ may each independently be a substituted or unsubstituted C₅-C₃₀ carbocyclic group, a substituted or unsubstituted C₁-C₃₀ heterocyclic group, or any combination thereof,

a1 and a2 may each independently be an integer from 0 to 3,

wherein when a1 is 2 or more, two or more of L₁(s) may be identical to or different from each other, and when a2 is 2 or more, two or more of L₂(s) may be identical to or different from each other,

R₁, R₂, R₁₀, R₂₀, R₃₀, and R₄₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), —P(═O)(Q₈)(Q₉), or any combination thereof,

b1 and b2 may each independently be an integer from 1 to 5,

wherein when b1 is 2 or more, two or more of R₁(s) may be identical to or different from each other, and when b2 is 2 or more, two or more of R₂ may be identical to or different from each other,

b10 and b20 may each independently be an integer from 1 to 8,

b30 and b40 may each independently be an integer from 1 to 3,

c1 and c2 may each independently be an integer from 1 to 8,

the sum of b10 and c1 may be 9 and the sum of b20 and c2 may be 9, and

at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, or any combination thereof,

a C₁-C₆₀ alkyl group, a C₂-C₆₀ alkenyl group, a C₂-C₆₀ alkynyl group, a C₁-C₆₀ alkoxy group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇), or any combination thereof,

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or any combination thereof,

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), —B(Q₂₆)(Q₂₇), or any combination thereof, or

—Si(Q₃₁)(Q₃₂)(Q₃₃), —N(Q₃₄)(Q₃₅), —B(Q₃₆)(Q₃₇), or any combination thereof,

wherein Q₁ to Q₇, Q₁₁ to Q₁₇, Q₂₁ to Q₂₇, and Q₃₁ to Q₃₇ may each independently be hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, or any combination thereof.

An organic light-emitting device according to another aspect includes a first electrode, a second electrode, and an organic layer between the first electrode and the second electrode and including at least one heterocyclic compound.

### BRIEF DESCRIPTION OF THE DRAWING

The above and other aspects, features, and advantages of certain embodiments of the disclosure will be more apparent from the following description taken in conjunction with FIGURE which shows a schematic cross-sectional view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween In contrast, when an element is referred to as being “directly on” another element, there are no intervening elements present

It will be understood that, although the terms “first,” “second,” “third” etc. may be used herein to describe various elements, components, regions, layers and/or sections, these elements, components, regions, layers and/or sections should not be limited by these terms These terms are only used to distinguish one element, component, region, layer or section from another element, component, region, layer or section Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” As used herein, the term “and/or” includes any and all combinations of one or more of the associated listed items It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures It will be understood that relative terms are intended to encompass different orientations of the device in addition to the orientation depicted in the Figures For example, if the device in one of the figures is turned over, elements described as being on the “lower” side of other elements would then be oriented on “upper” sides of the other elements The exemplary term “lower,” can therefore, encompasses both an orientation of “lower” and “upper,” depending on the particular orientation of the figure Similarly, if the device in one of the figures is turned over, elements described as “below” or “beneath” other elements would then be oriented “above” the other elements The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments As such, variations from the shapes of the illustrations as a result, for example, of manufacturing techniques and/or tolerances, are to be expected Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features Moreover, sharp angles that are illustrated may be rounded Thus, the regions illustrated in the figures are schematic in nature and their shapes are not intended to illustrate the precise shape of a region and are not intended to limit the scope of the present claims.

A heterocyclic compound according to an embodiment of the present disclosure may be represented by Formula 1:

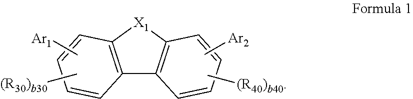

X₁ in Formula 1 may be O or Se.

In one or more embodiments, X₁ may be O. In one or more embodiments, X₁ may be Se.

In Formula 1, Ar₁ may be a group represented by Formula 1A, and Are may be a group represented by Formula 1B.

In one or more embodiments, Ar₂ may be a group represented by one of Formulae 1A-1 to 1A-5:

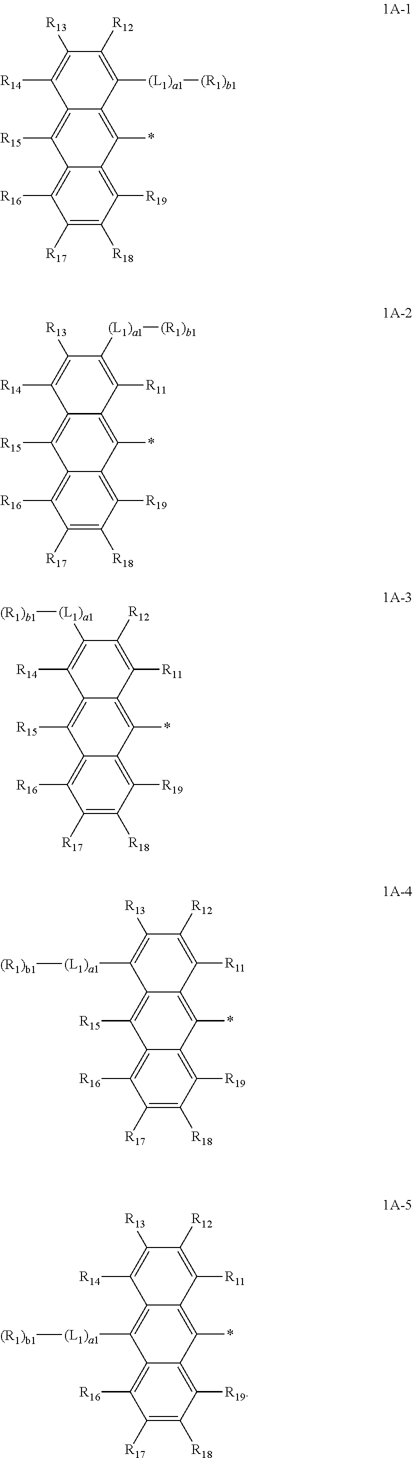

In Formulae 1A-1 to 1A-5,
L₁, a1, R₁, and b1 may be the same as described in the present specification,
R₁₁ to R₁₉ may be the same as described in connection with R₁₀, and
* indicates a binding site to a neighboring atom.

In one or more embodiments, Ar₂ may be a group represented by one of Formulae 2A-1 to 2A-5:

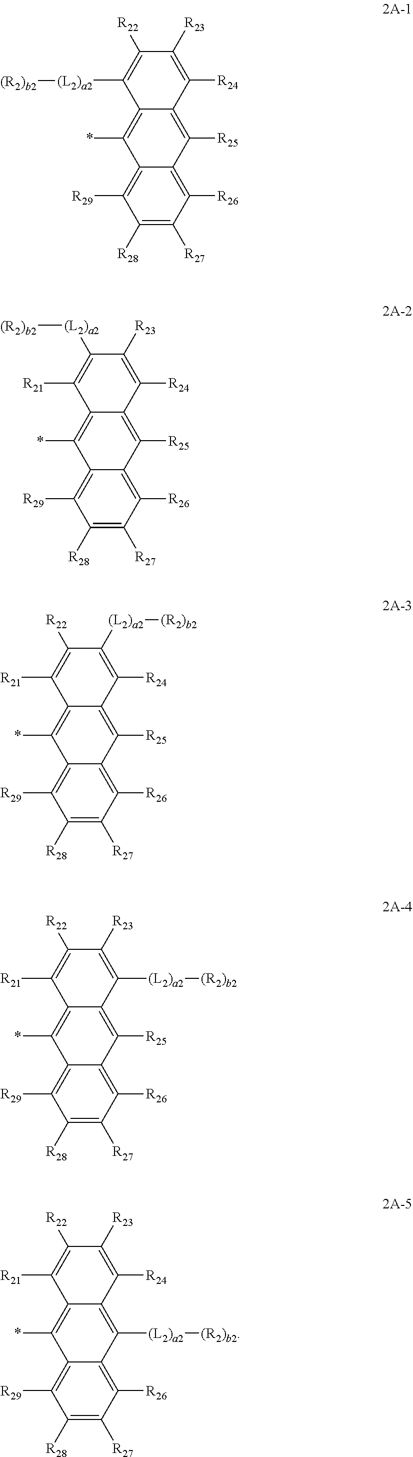

In Formulae 2A-1 to 2A-5,
L₂, a2, R₂, and b2 may be the same as described in the present specification,
R₂₁ to R₂₉ may be the same as described in connection with R₂₀, and
* indicates a binding site to a neighboring atom.

In one or more embodiments, the heterocyclic compound represented by Formula 1 may be a compound represented by one of Formulae 1-1 to 1-6:

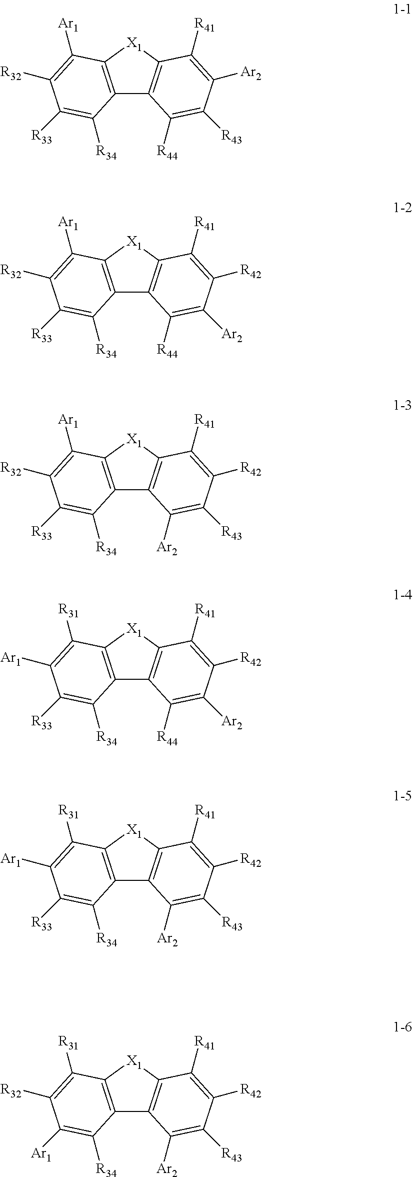

In Formulae 1-1 to 1-6,
X₁, Ar₁ and Ar₂ may be the same as described in the present specification,
R₃₁ to R₃₄ may be the same as described in connection with R₃₀,
R₄₁ to R₄₄ may be the same as described in connection with R₄₀.

L₁ and L₂ in Formulae 1A and 1B may each independently be a substituted or unsubstituted C₅-C₃₀ carbocyclic group, a substituted or unsubstituted C₁-C₃₀ heterocyclic group, or any combination thereof.

In one or more embodiments, L₁ and L₂ may each independently be: a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, a pentacenylene group, or any combination thereof; or

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, a pentacenylene group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or any combination thereof.

a1 and a2 in Formulae 1A and 1B may each independently be an integer from 0 to 3. When a1 is 2 or more, two or more of L₁(s) may be identical to or different from each other. When a2 is 2 or more, two or more of L₂(s) may be identical to or different from each other.

R₁, R₂, R₁₀, R₂₀, R₃₀, and R₄₀ in Formulae 1, 1A, and 1B may each independently be hydrogen, deuterium, —F, —Cl, —Br, —I, —SF₅, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), —P(═O)(Q₈)(Q₉), or any combination thereof.

In one or more embodiments, R₁, R₂, R₁₀, R₂₀, R₃₀, and R₄₀ may be each independently:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkoxy group, or any combination thereof;

a C₁-C₂₀ alkyl group, a C₁-C₂₀ alkoxy group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocartazolyl group, an imidazopyridinyl group, an imidazopyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof; or

—N(Q₁)(Q₂), —Si(Q₃)(Q₄)(Q₅), —B(Q₆)(Q₇), —P(═O)(Q₈)(Q₉), or any combination thereof,

wherein Q₁ to Q₉ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CH₃, —CD₂CD₃, —CD₂CD₂H, —CD₂CDH₂, or any combination thereof;

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, or any combination thereof; or

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, or any combination thereof, each substituted with at least one deuterium, a C₁-C₁₀ alkyl group, a phenyl group, or any combination thereof.

b1 and b2 in Formulae 1, 1A, and 1B may each independently be an integer from 1 to 5. When b1 is 2 or more, two or more of R₁(s) may be identical to or different from each other. When b2 is 2 or more, two or more of R₂(s) may be identical to or different from each other.

b10 and b20 in Formulae 1, 1A and 1B may each independently be an integer from 1 to 8. When b10 is 2 or more, two or more of R₁₀(s) may be identical to or different from each other. When b20 is 2 or more, two or more of R₂₀(s) may be identical to or different from each other.

b30 and b40 in Formulae 1, 1A and 1B may each independently be an integer from 1 to 3. When b30 is 2 or more, two or more of R₃₀(s) may be identical to or different from each other. When b40 is 2 or more, two or more of R₄₀(s) may be identical to or different from each other.

c1 and c2 in Formulae 1, 1A and 1B may each independently be an integer from 1 to 8. When c1 is 2 or more, two or more of-(L₁)ₐ₁-(R₁)_{b1} group(s) may be identical to or different from each other. When c2 is 2 or more, two or more of-(L₂)ₐ₂-(R₂)_{b2} group(s) may be identical to or different from each other.

In Formulae 1, 1A, and 1B, the sum of b10 and c1 may be 9 and the sum of b20 and c2 may be 9.

In one or more embodiments, b10 may be 8 and c1 may be 1.

In one or more embodiments, b20 may be 8 and c2 may be 1.

In one or more embodiments, R₁ and R₂ may each independently be deuterium, —F, —Cl, —Br, —I, —CF₃, —SF₅, a hydroxyl group, a cyano group, a nitro group, —SF₅, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), —P(═O)(Q₈)(Q₉), or any combination thereof.

In one or more embodiments, R₁ and R₂ may each independently be deuterium, —F, —CF₃, a cyano group, a nitro group, —SF₅, a group represented by Formulae 9-1 to 9-19, a group represented by Formulae 10-1 to 10-208, or any combination thereof:

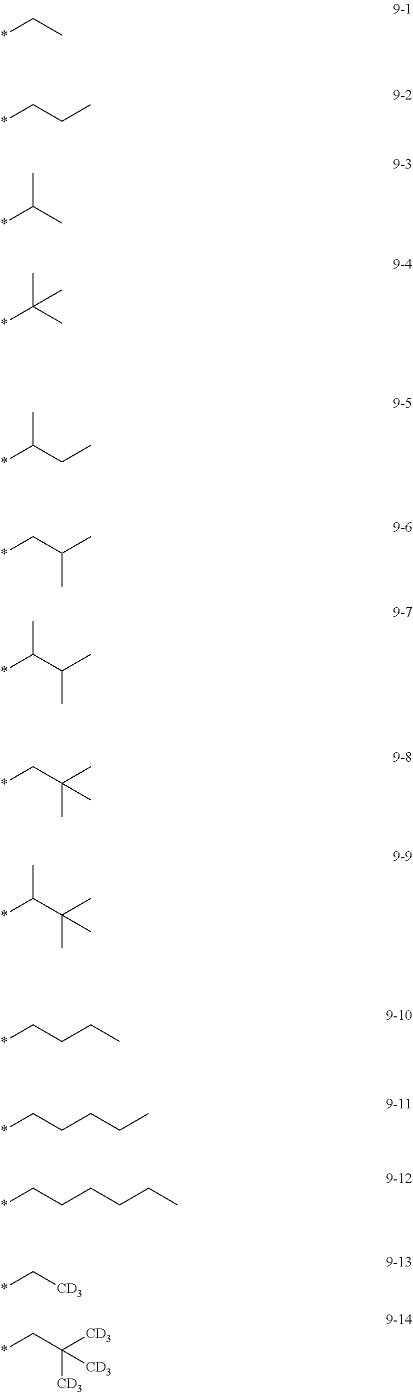

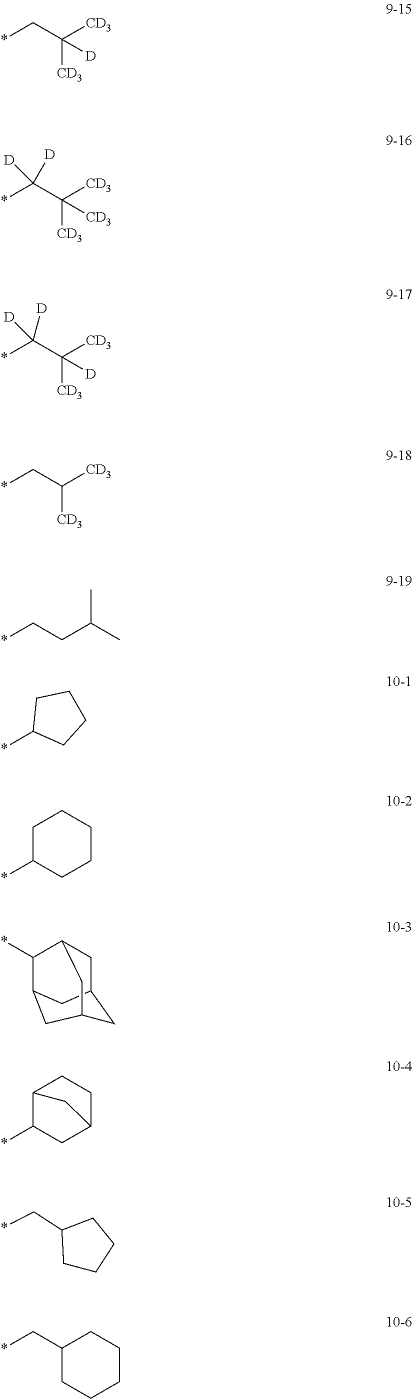

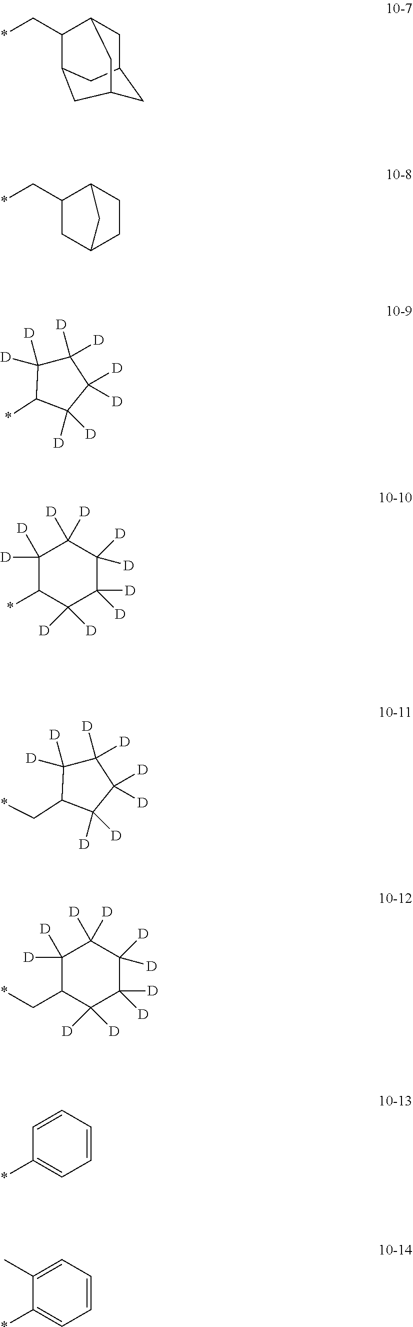

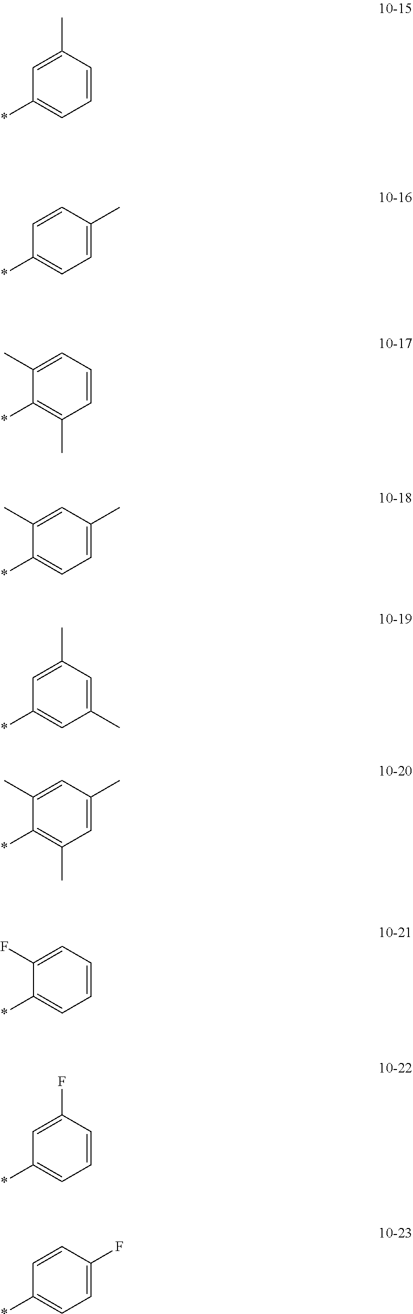

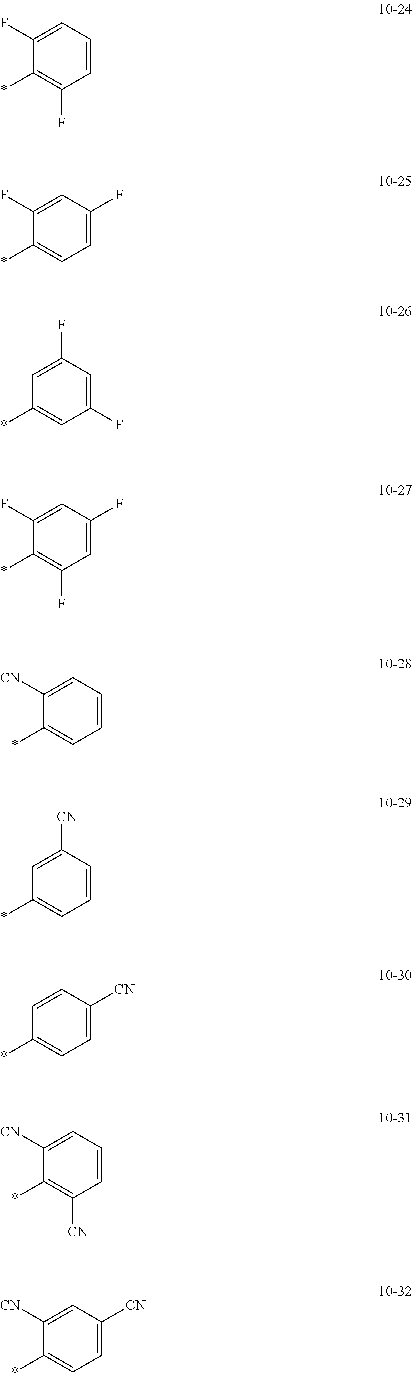

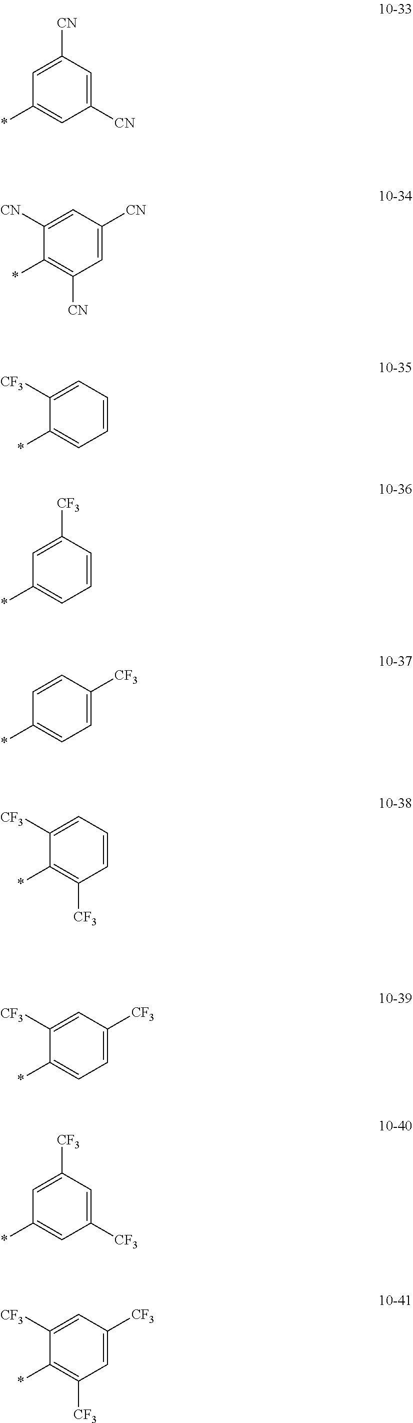

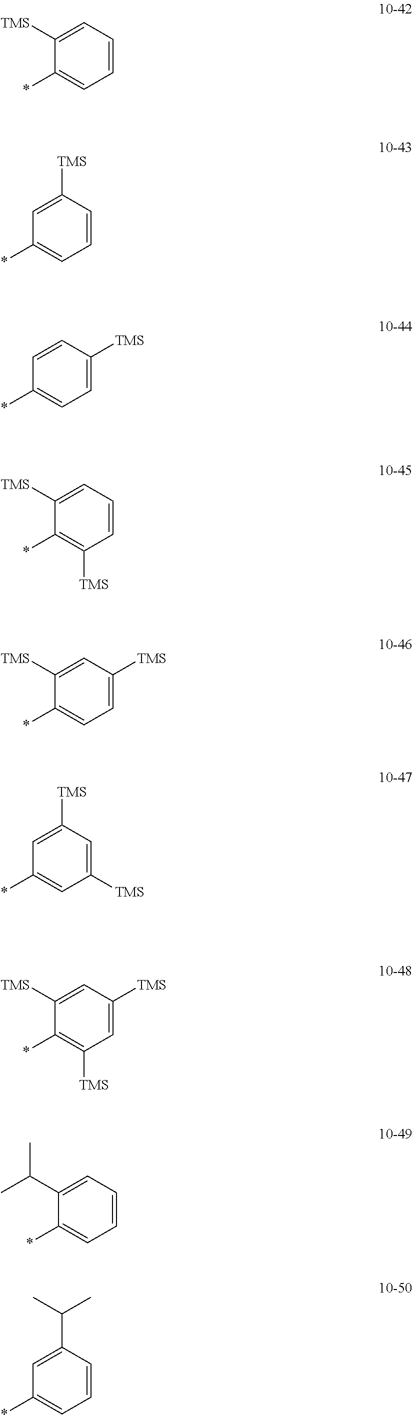

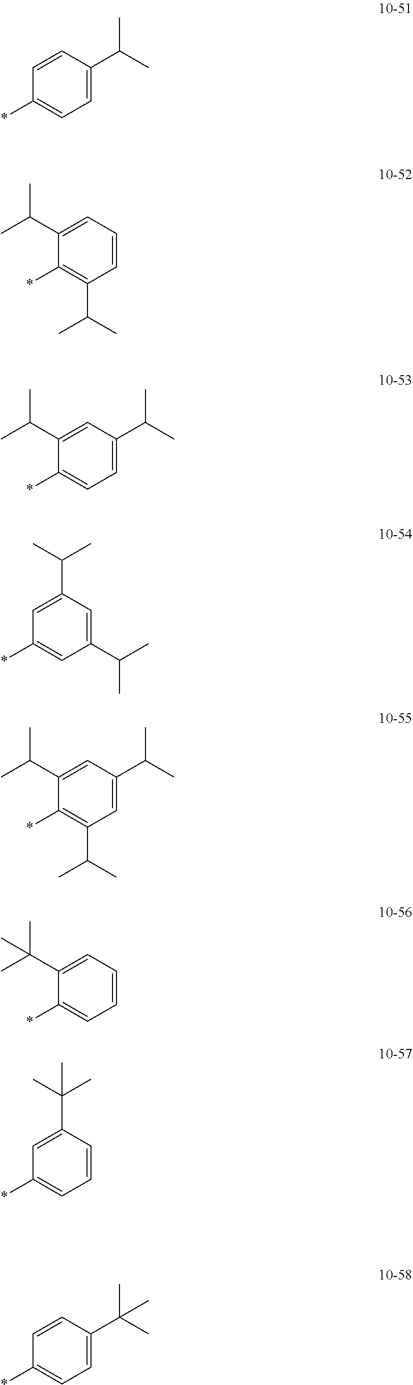

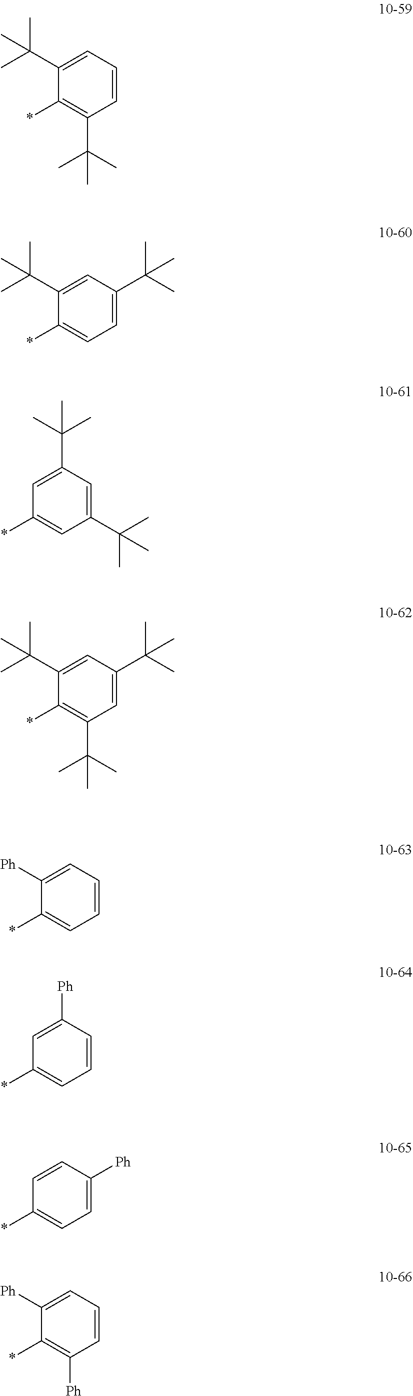

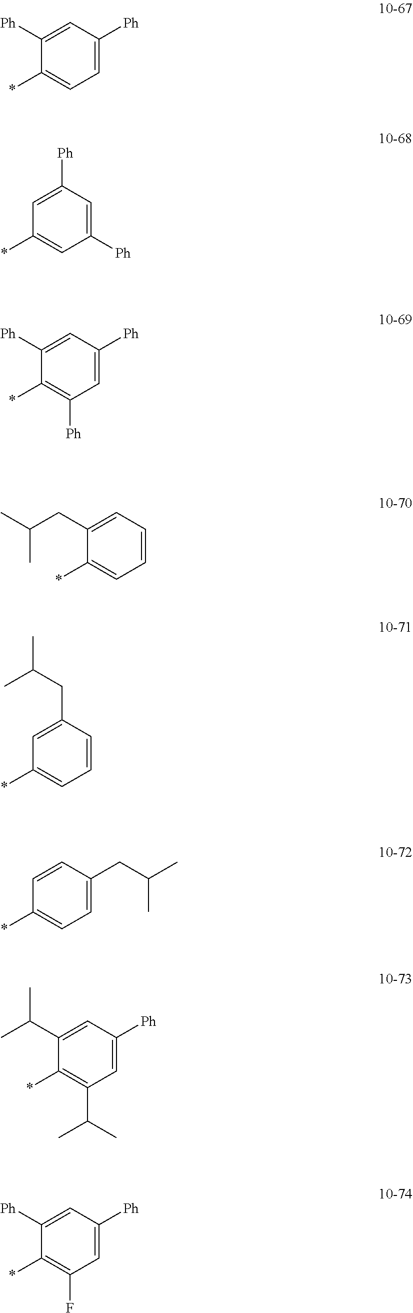

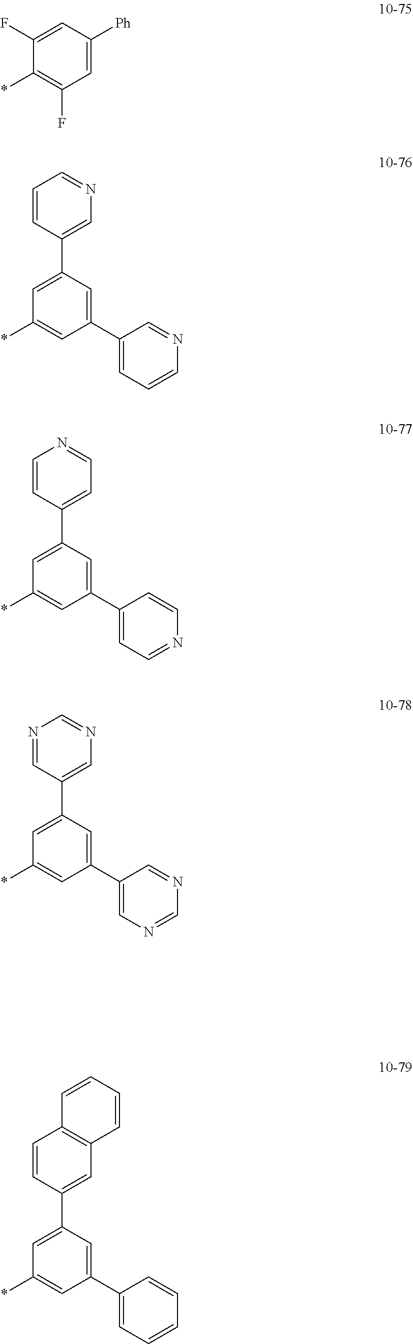 [SMILES: *c1c(F)cc(-c2ccccc2)cc1F ; *c1cc(-c2cccnc2)cc(-c2cccnc2)c1 ; *c1cc(-c2ccncc2)cc(-c2ccncc2)c1 ; *c1cc(-c2cncnc2)cc(-c2cncnc2)c1 ; *c1cc(-c2ccccc2)cc(-c2ccc3ccccc3c2)c1]

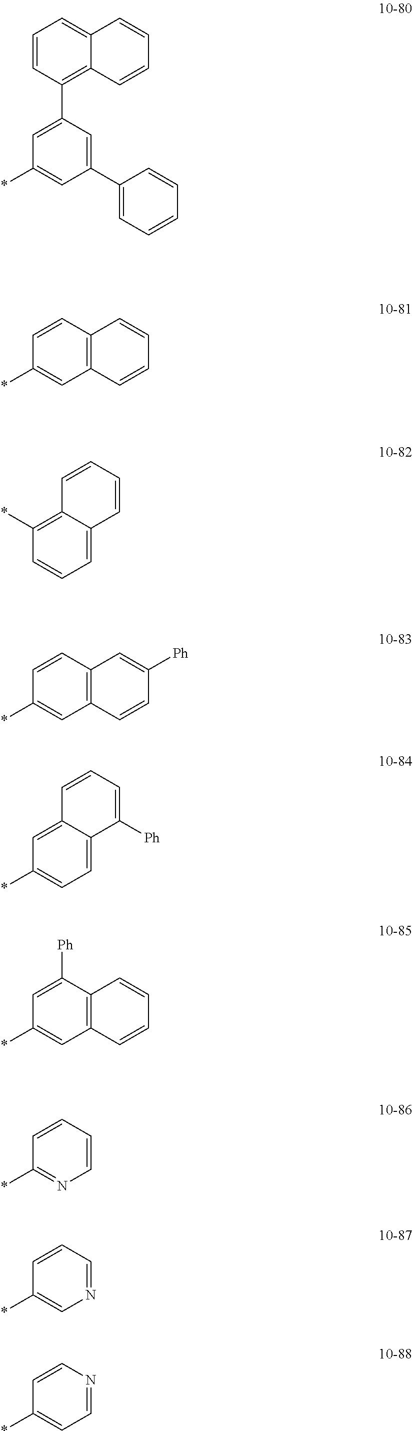

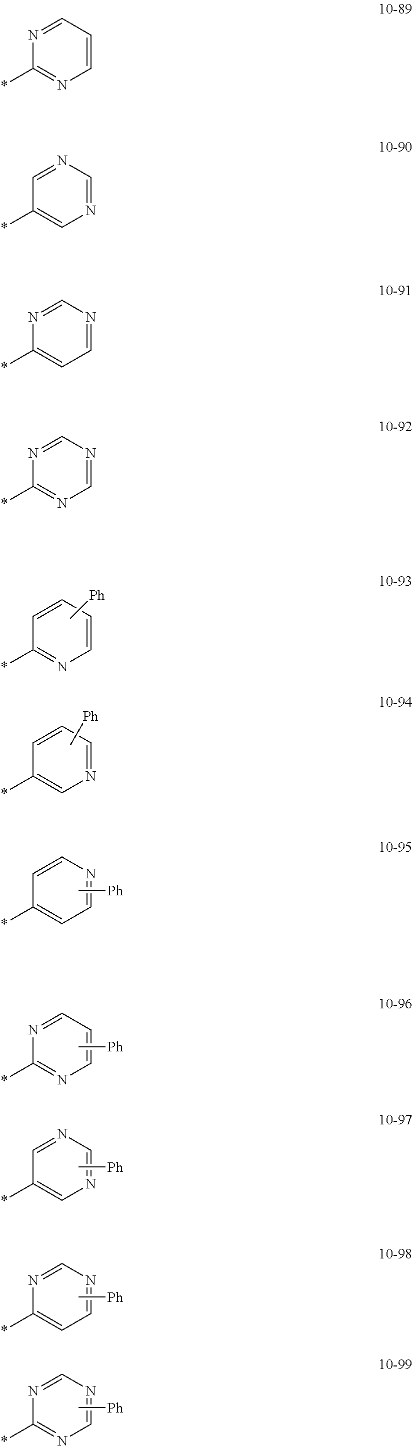

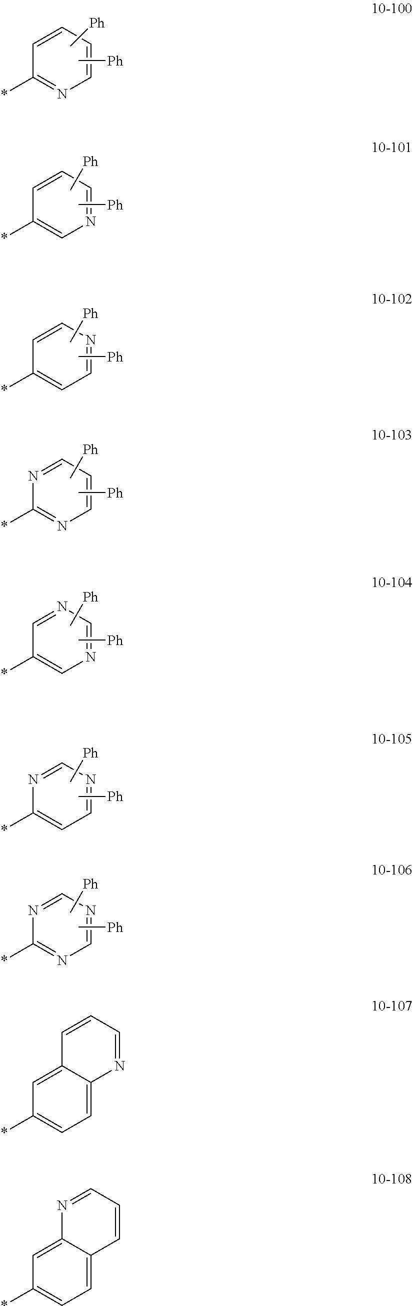

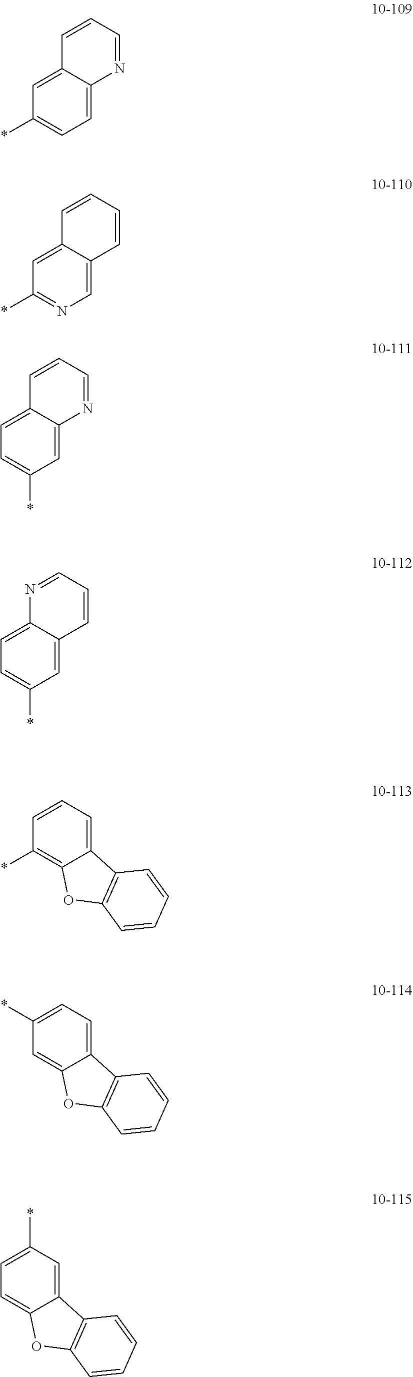

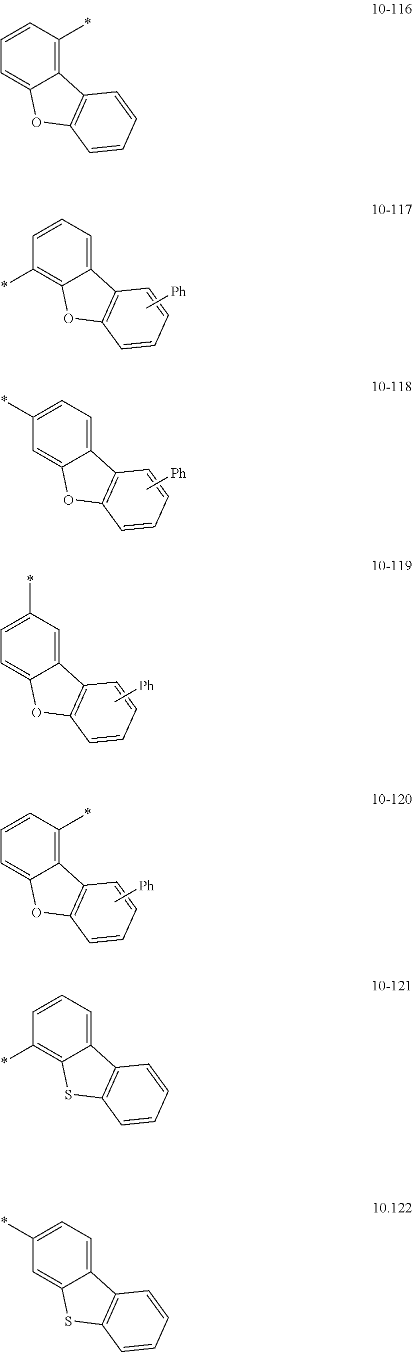

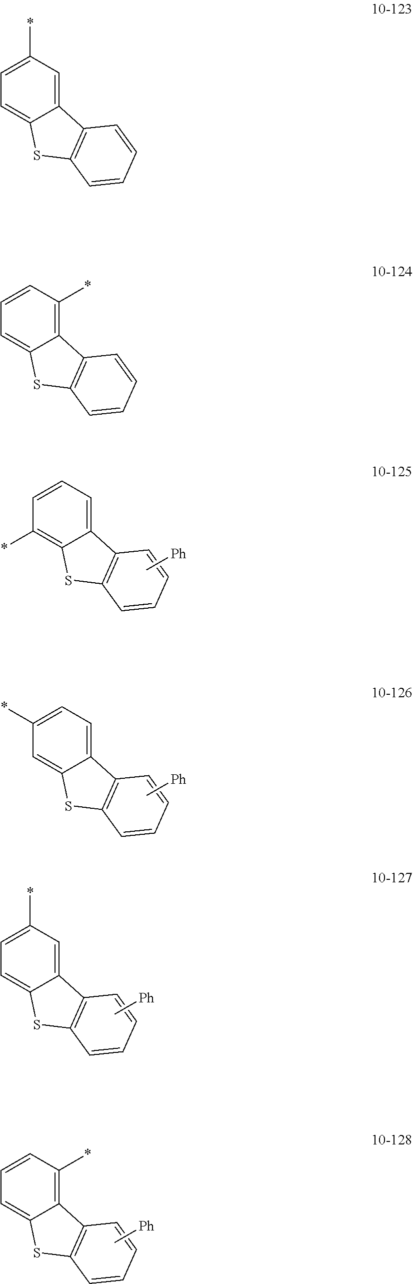

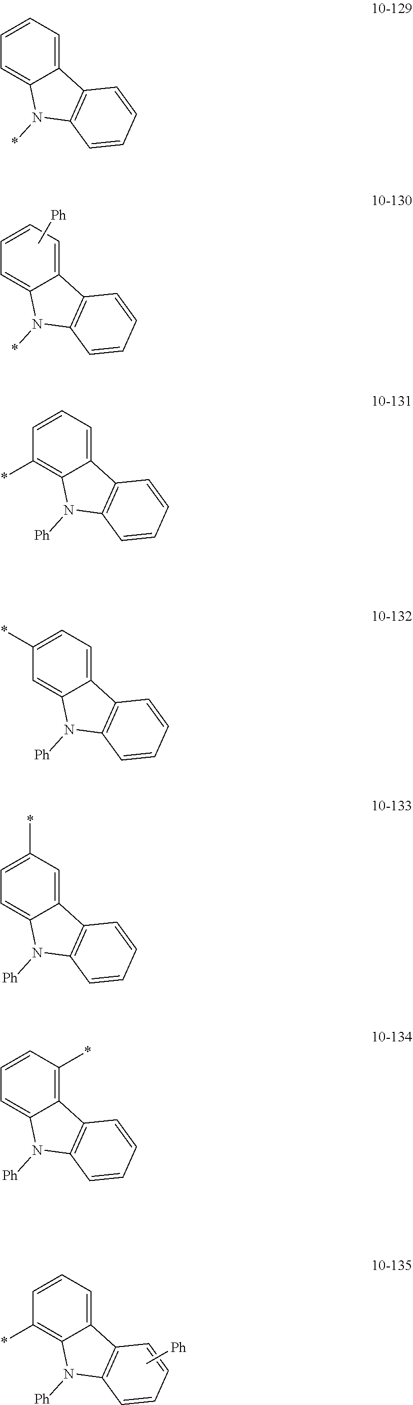

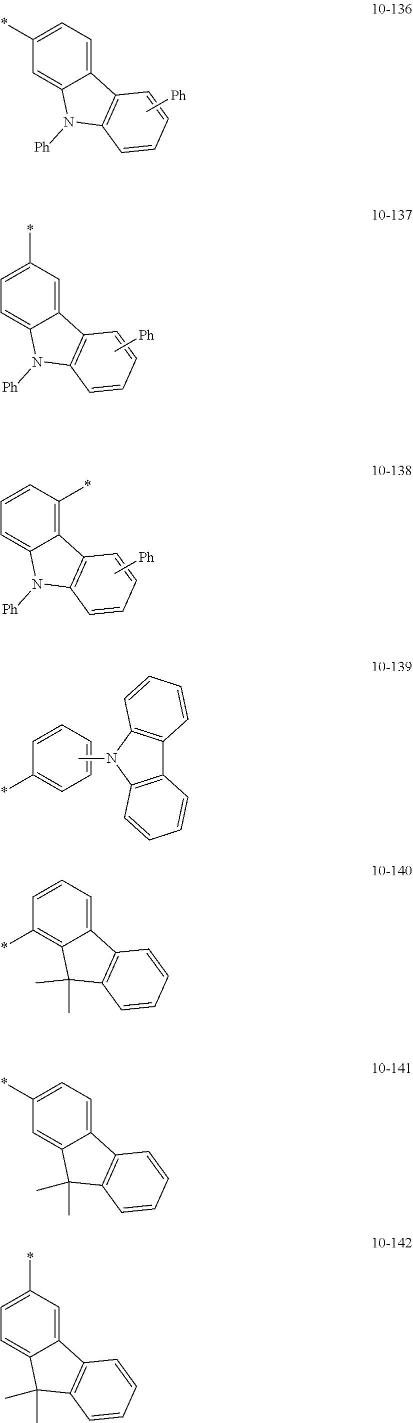

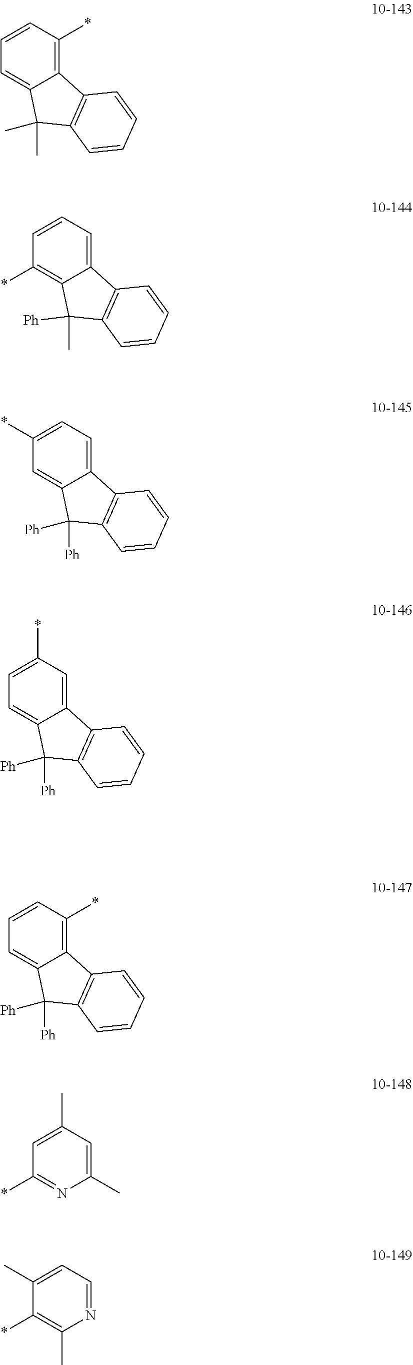

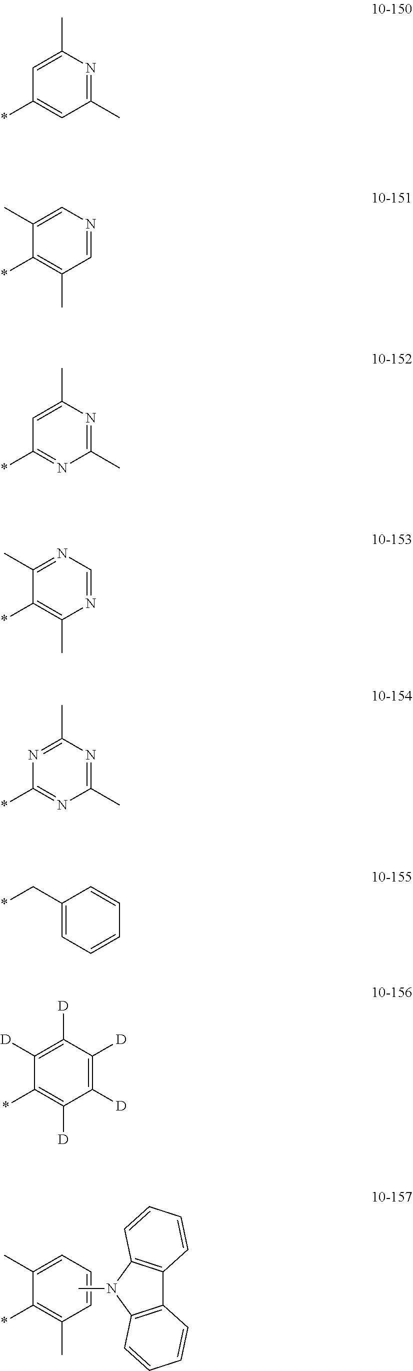

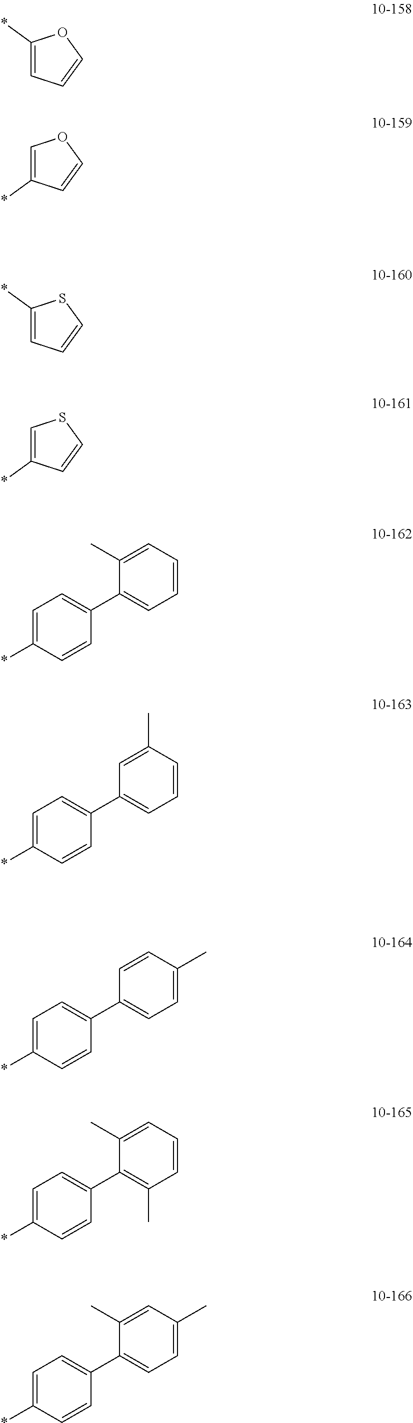

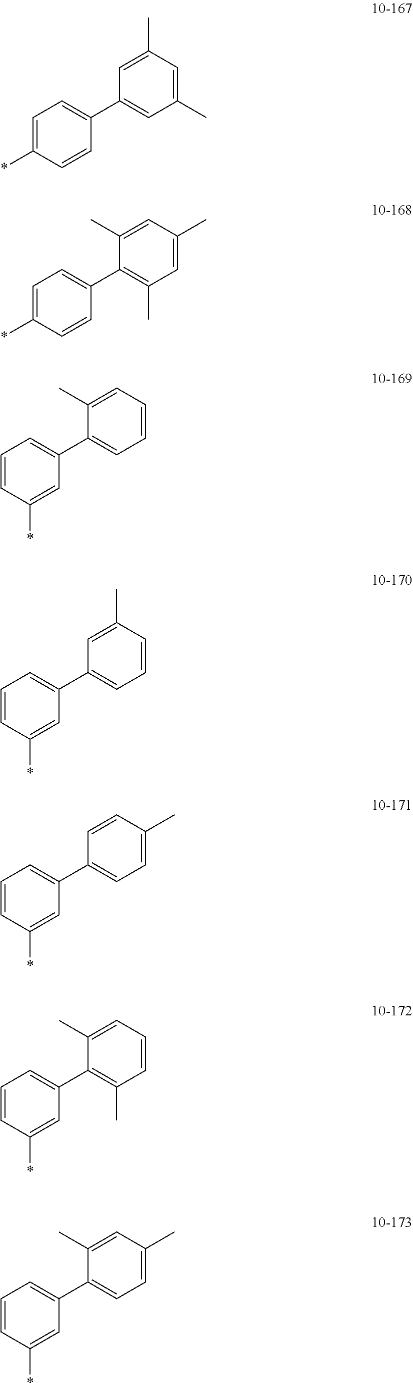

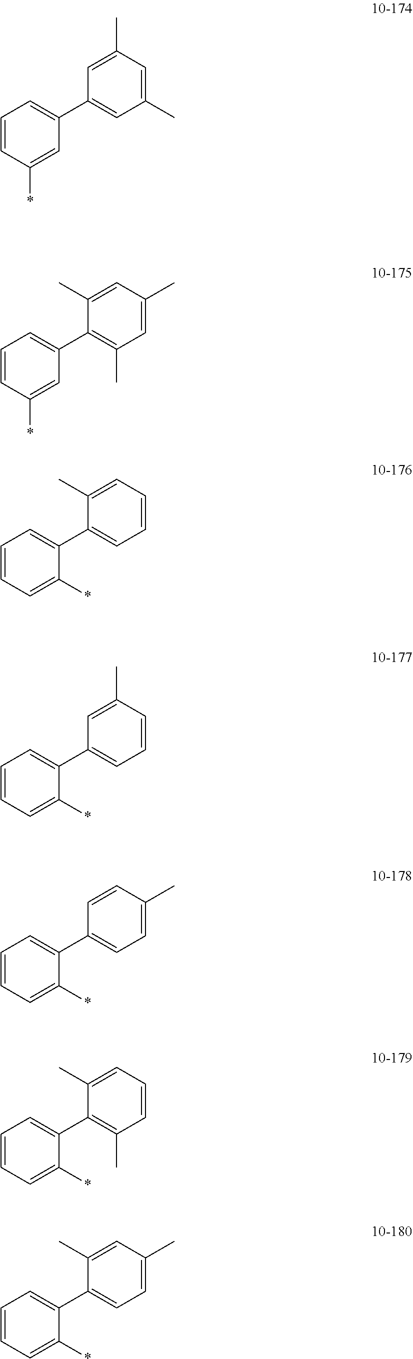

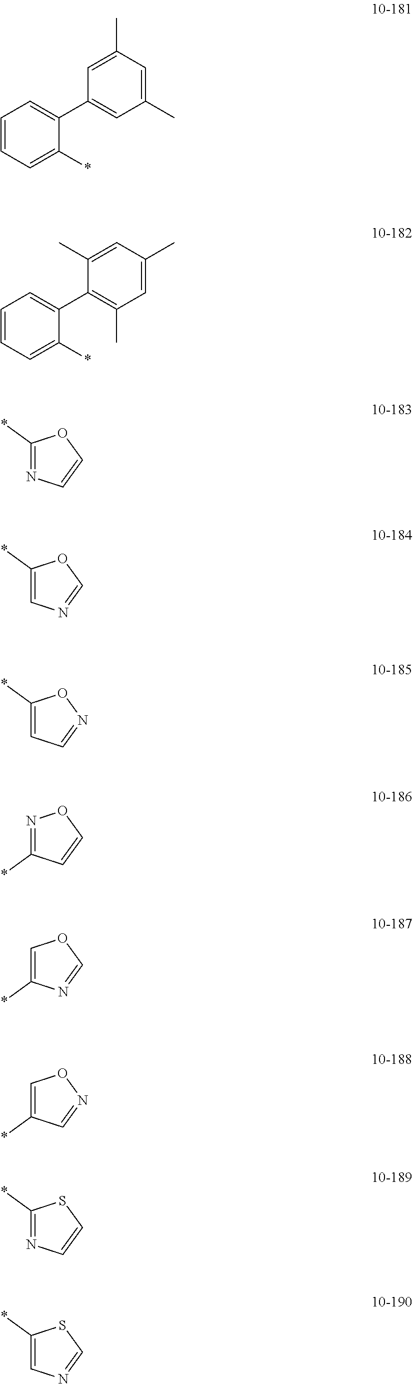

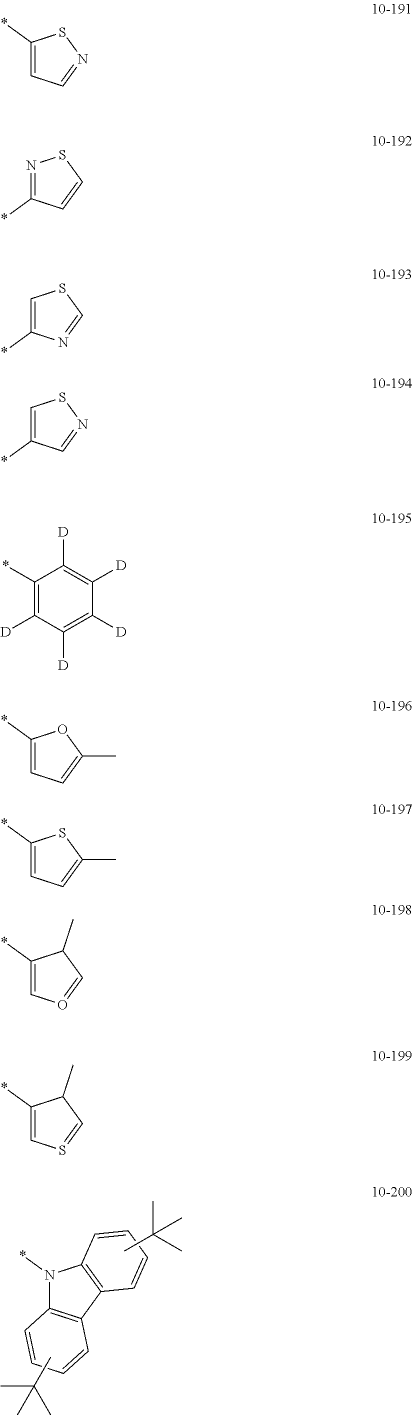

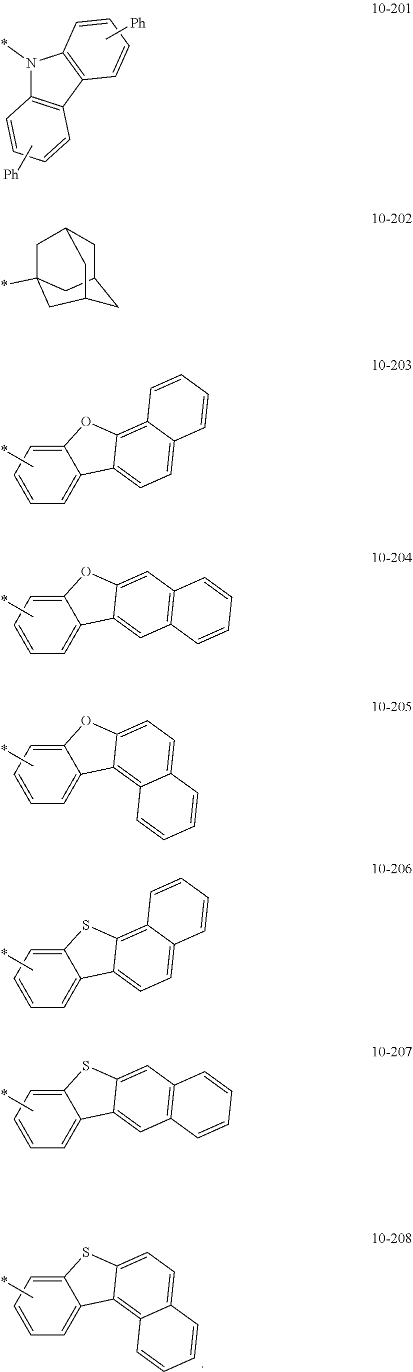

In Formulae 9-1 to 9-19 and 10-1 to 10-208, * indicates a binding site to a neighboring atom, Ph may be a phenyl group, and TMS may be a trimethylsilyl group.

In one or more embodiments, R₁ and R₂ may each independently be: hydrogen, deuterium, —F, —Cl, —Br, —I, —CF₃, a hydroxyl group, a cyano group, a nitro group, —SF₅, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, or any combination thereof;

a C₁-C₂₀ alkyl group, a C₁-C₂₀ alkoxy group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a pyridinyl group, a pyrimidinyl group, a pyrazinyl group, a pyridazinyl group, a triazinyl group, or any combination thereof;

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyrimidinyl group, an imidazopyridinyl group, or any combination thereof; or

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a thazinyl group, a dibenzofuranyl group, a dibenzothiophenyl group, an imidazopyrimidinyl group, an imidazopyridinyl group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, or any combination thereof.

In one or more embodiments, R₁₀, R₂₀, R₃₀, and R₄₀ may each independently be hydrogen, deuterium, —F, —CF₃, a cyano group, a nitro group, —SF₅, —CH₃, —CD₃, —CD₂H, —CDH₂, —CF₃, —CF₂H, —CFH₂, a group represented by Formulae 9-1 to 9-19, a group represented by Formulae 10-1 to 10-208, or any combination thereof:

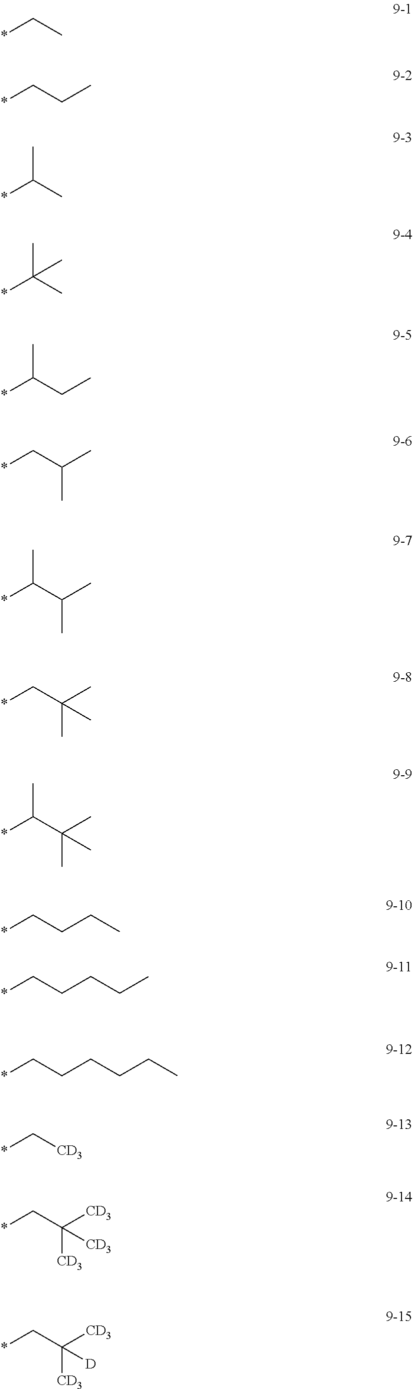

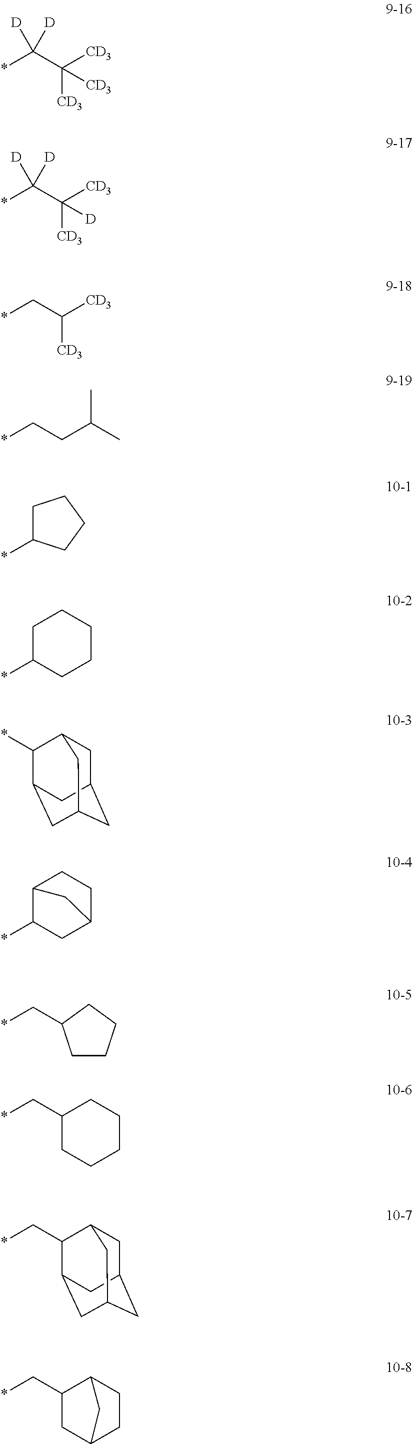

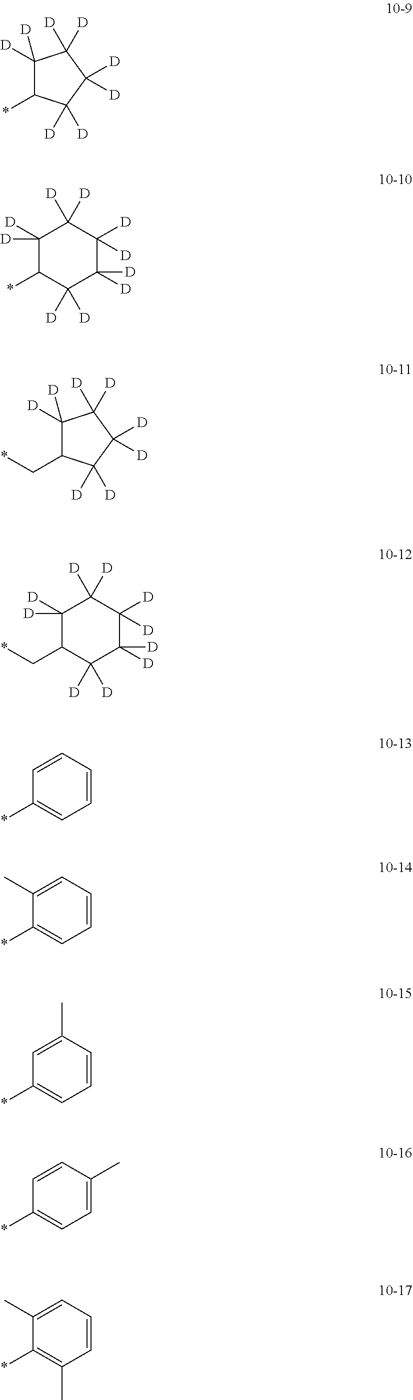

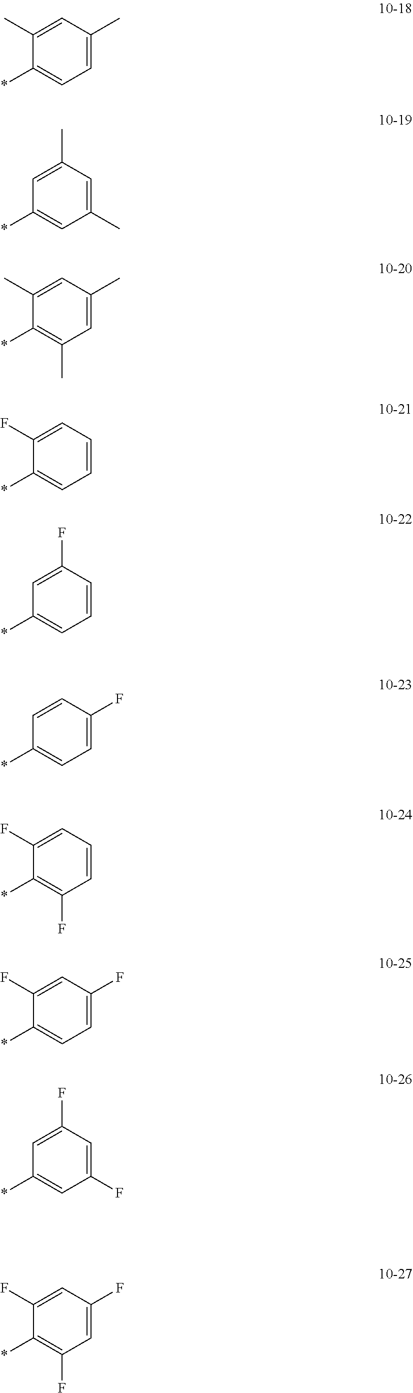

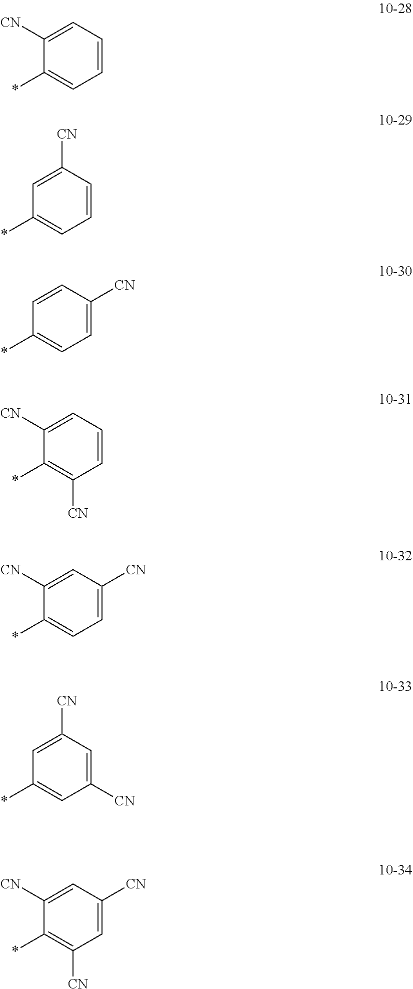

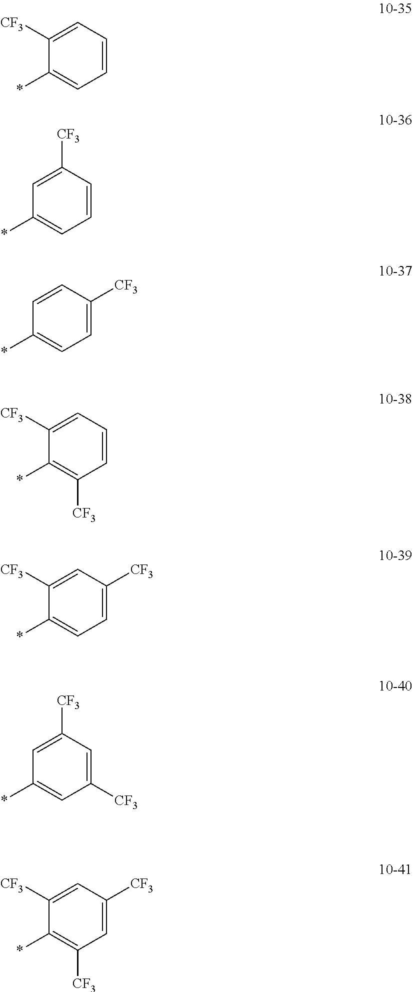

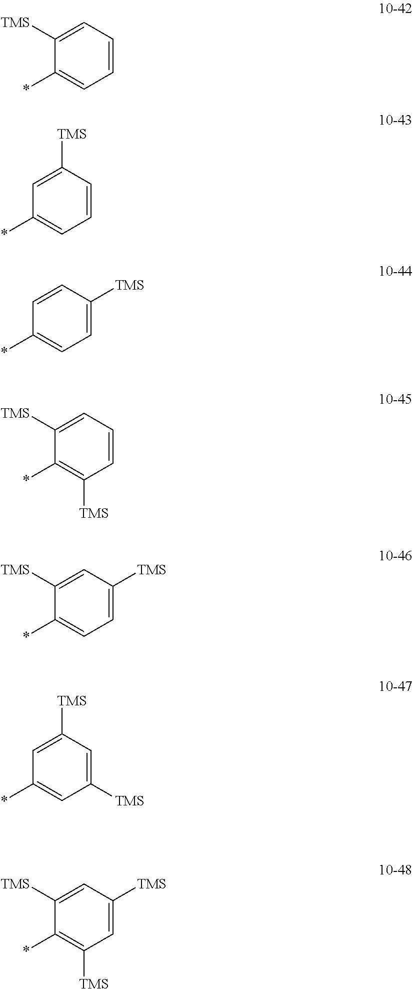

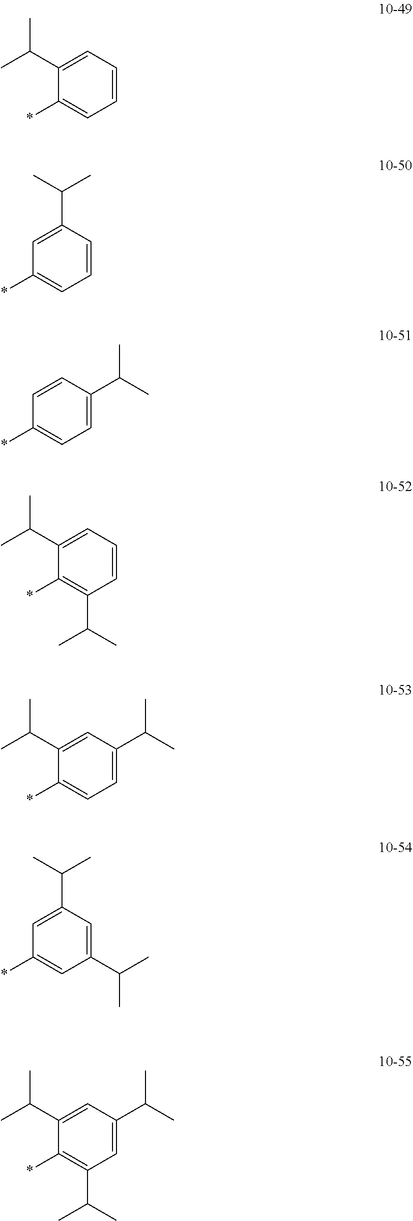

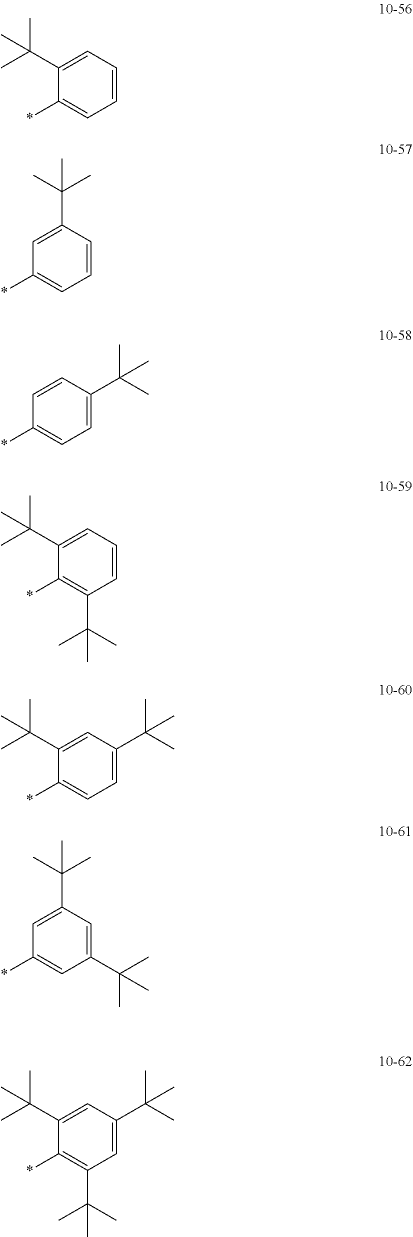

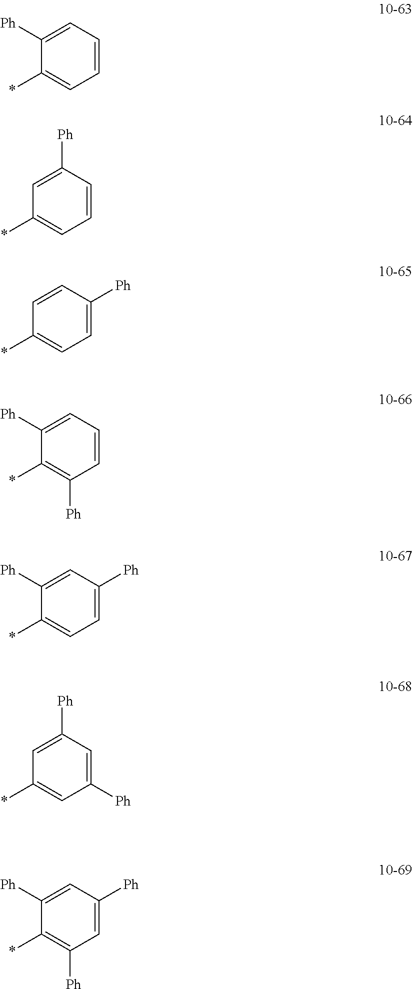

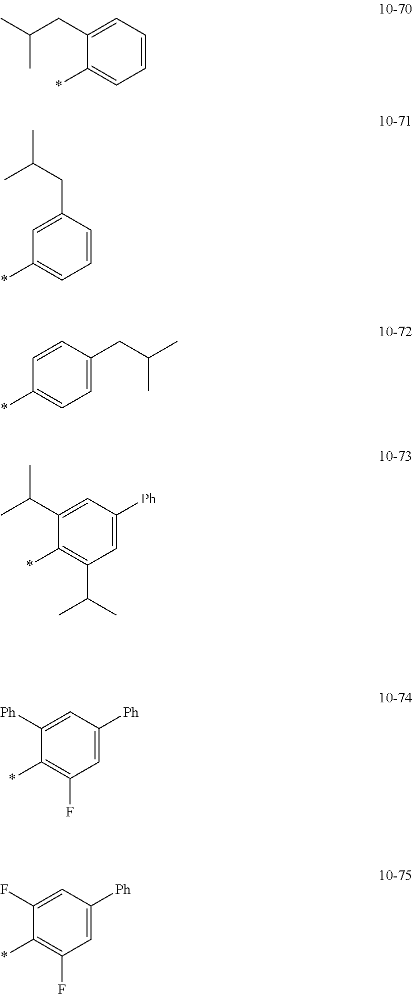

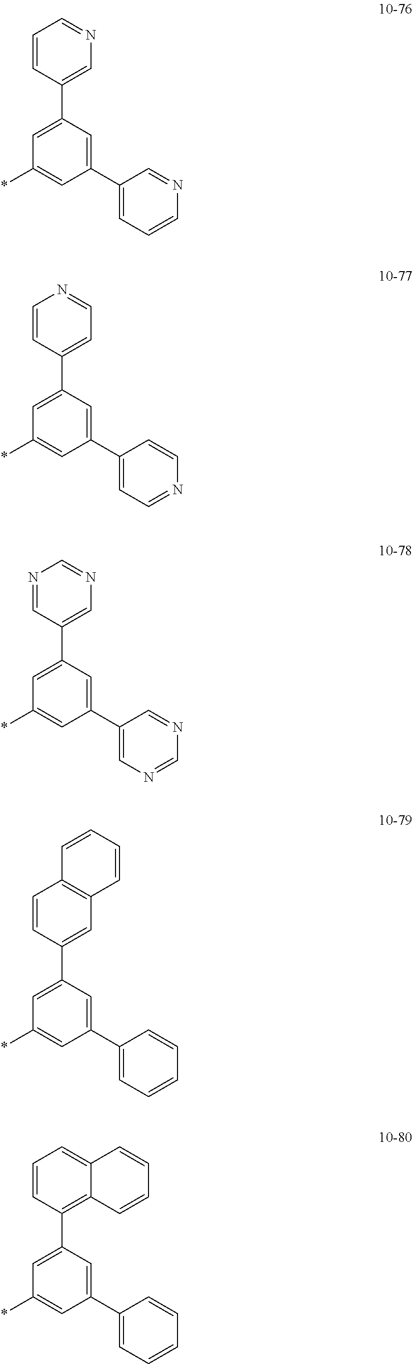 [SMILES: *c1cc(-c2cccnc2)cc(-c2cccnc2)c1 ; *c1cc(-c2ccncc2)cc(-c2ccncc2)c1 ; *c1cc(-c2cncnc2)cc(-c2cncnc2)c1 ; *c1cc(-c2ccccc2)cc(-c2ccc3ccccc3c2)c1 ; *c1cc(-c2ccccc2)cc(-c2cccc3ccccc23)c1]

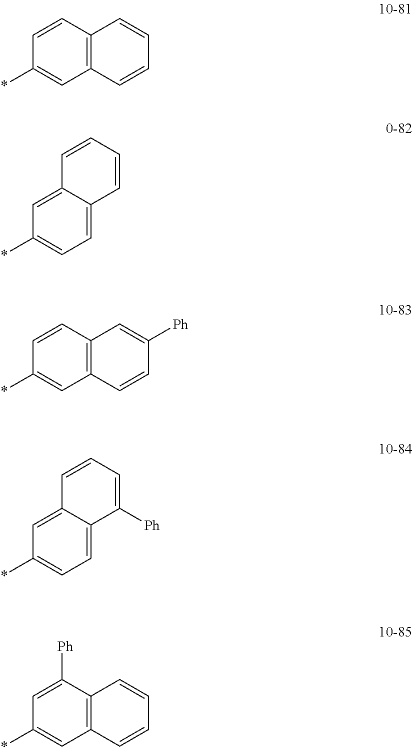 [SMILES: *c1ccc2ccccc2c1 ; *c1ccc2ccccc2c1 ; *c1ccc2cc(-c3ccccc3)ccc2c1 ; *c1ccc2c(-c3ccccc3)cccc2c1 ; *c1cc(-c2ccccc2)c2ccccc2c1]

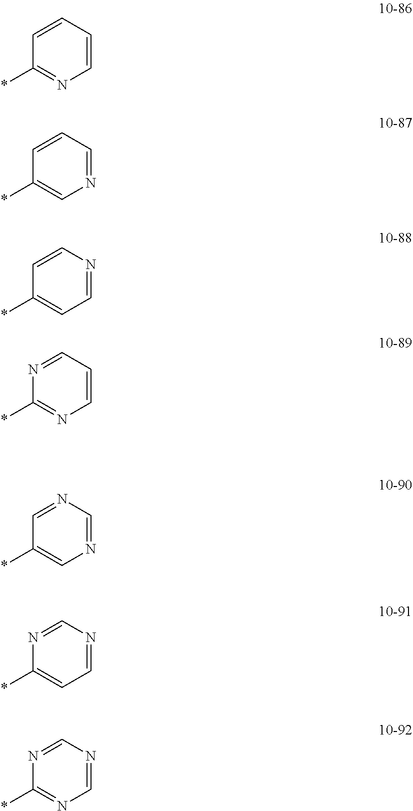

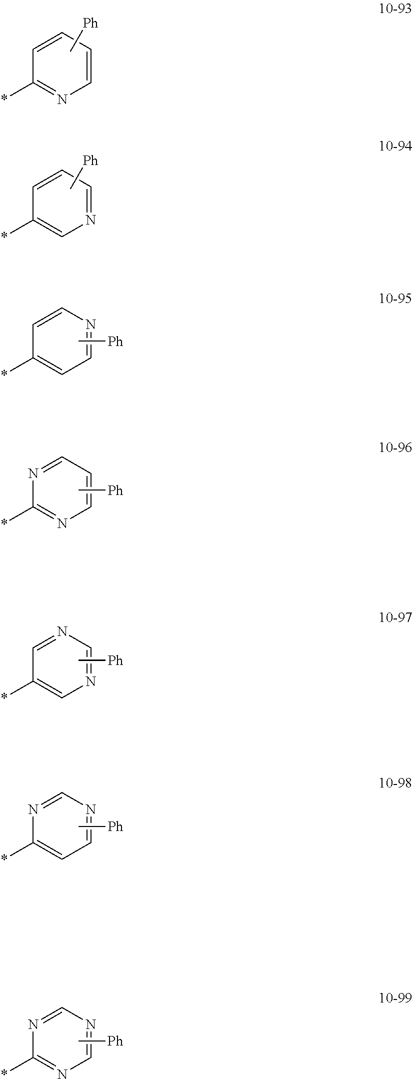

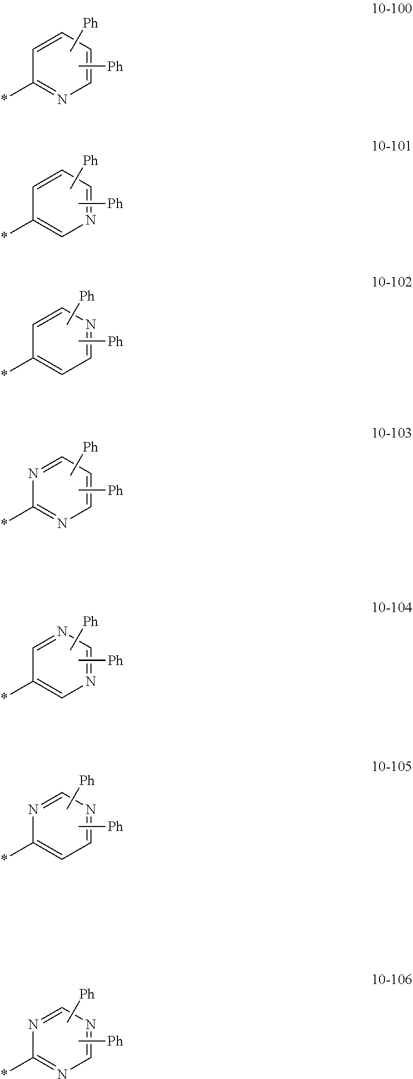

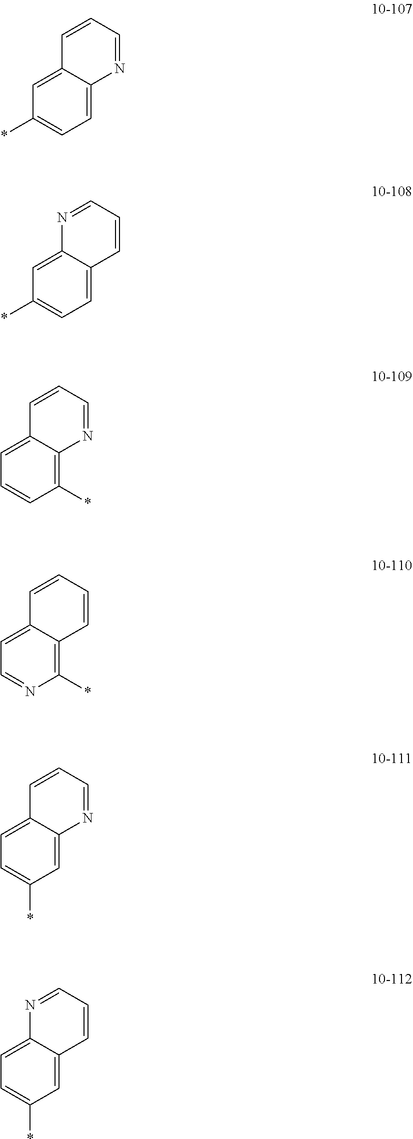 [SMILES: *c1ccc2ncccc2c1 ; *c1ccc2cccnc2c1 ; *c1cccc2cccnc12 ; *c1nccc2ccccc12 ; *c1ccc2cccnc2c1 ; *c1ccc2ncccc2c1]

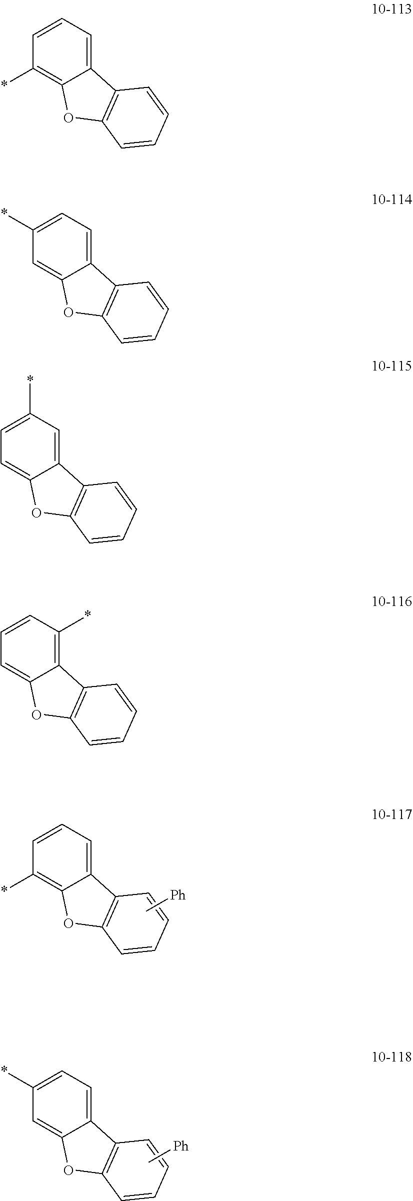

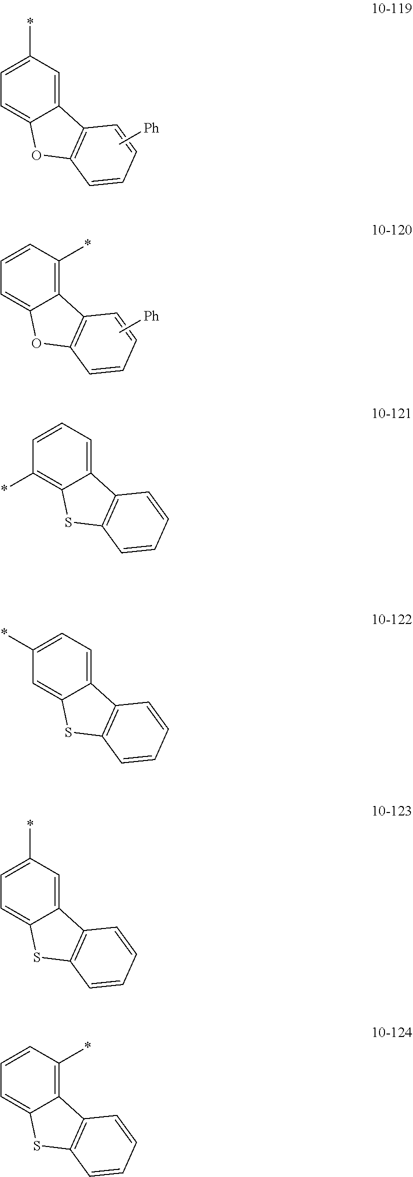

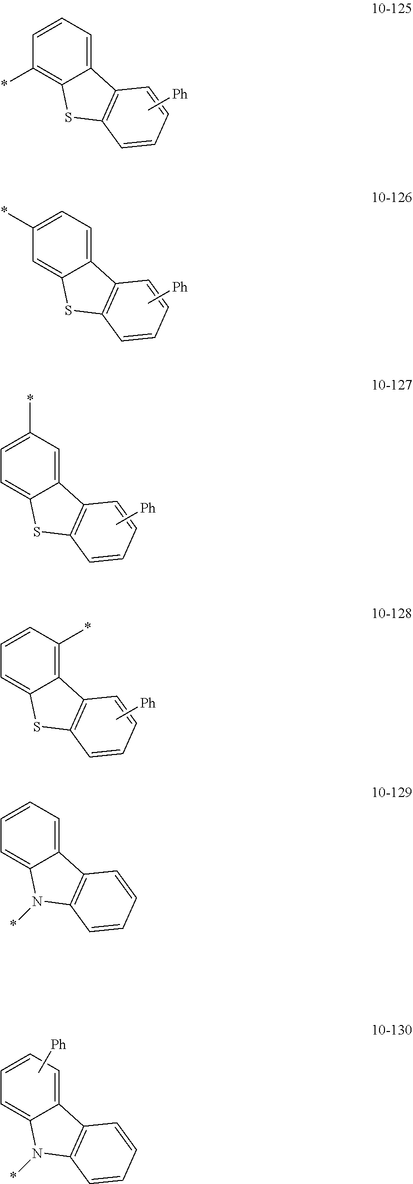

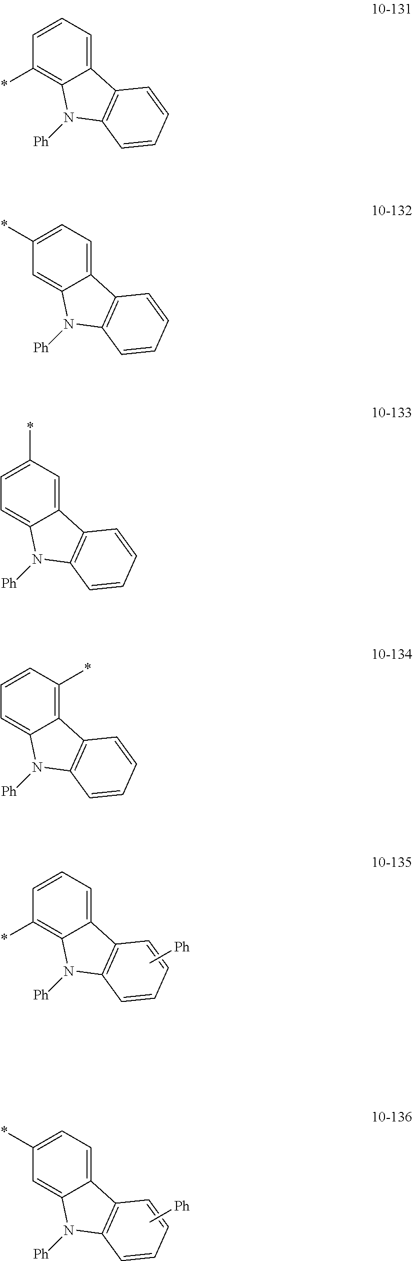

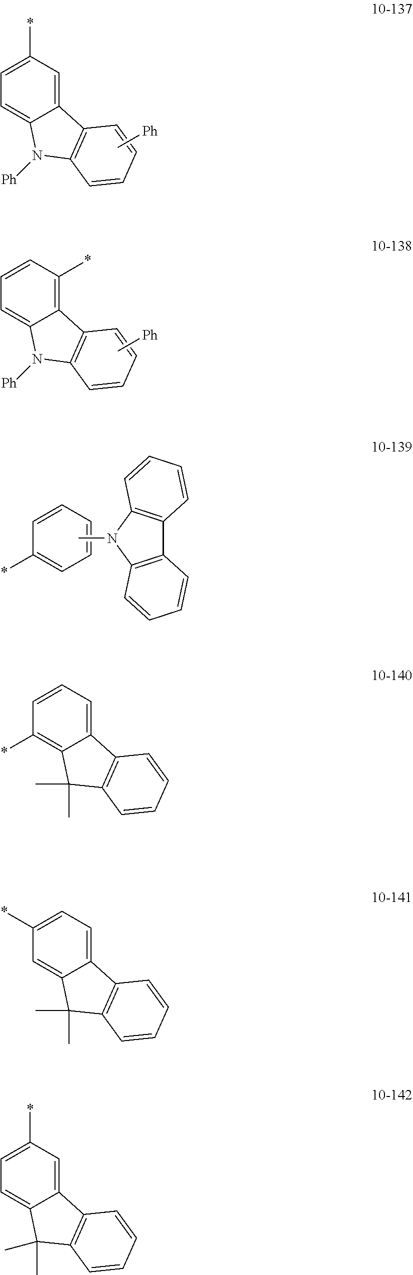

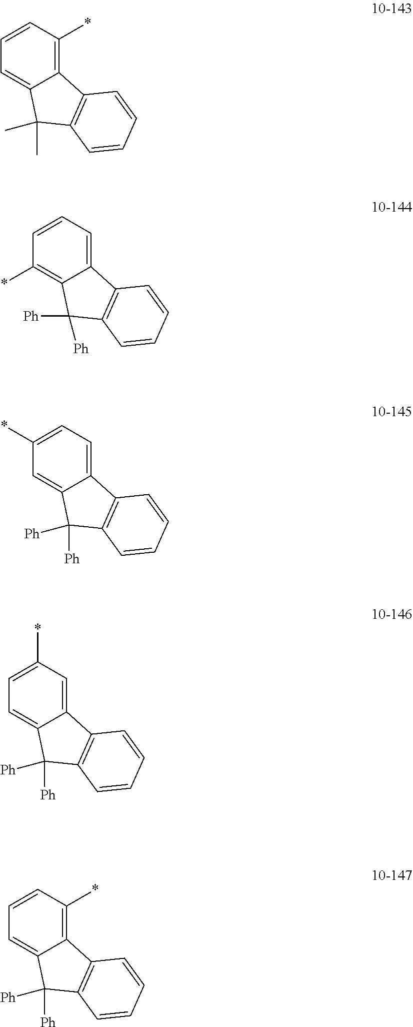 [SMILES: *c1cccc2c1-c1ccccc1C2(C)C ; *c1cccc2c1C(c1ccccc1)(c1ccccc1)c1ccccc1-2 ; *c1ccc2c(c1)C(c1ccccc1)(c1ccccc1)c1ccccc1-2 ; *c1ccc2c(c1)-c1ccccc1C2(c1ccccc1)c1ccccc1 ; *c1cccc2c1-c1ccccc1C2(c1ccccc1)c1ccccc1]

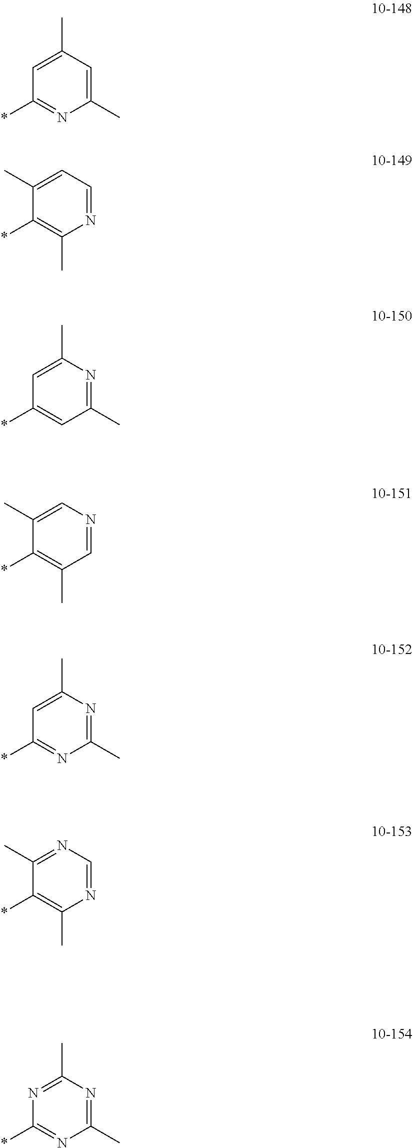

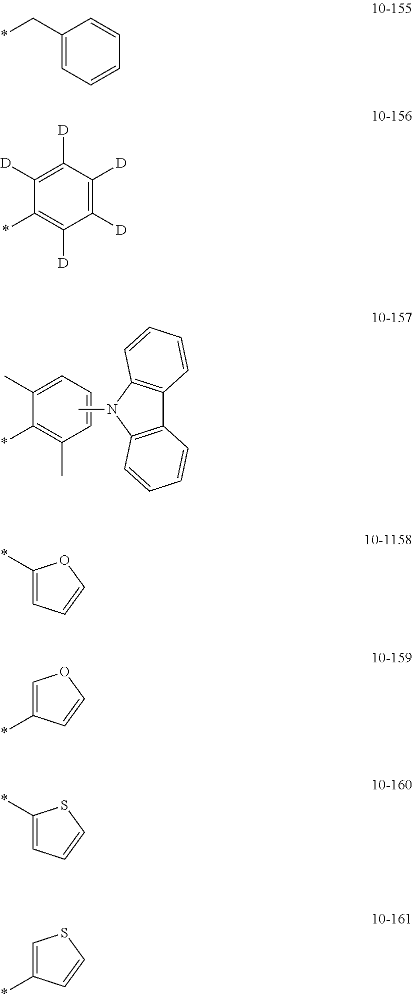

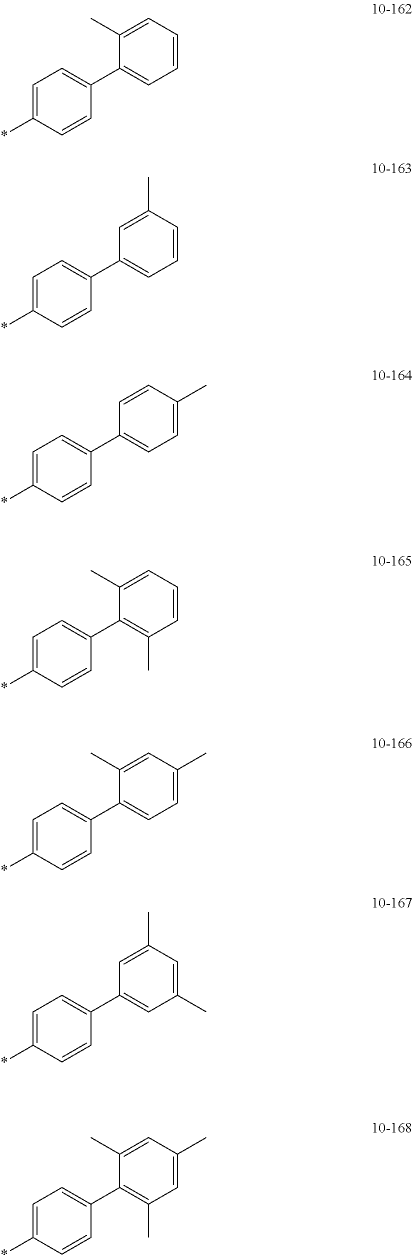

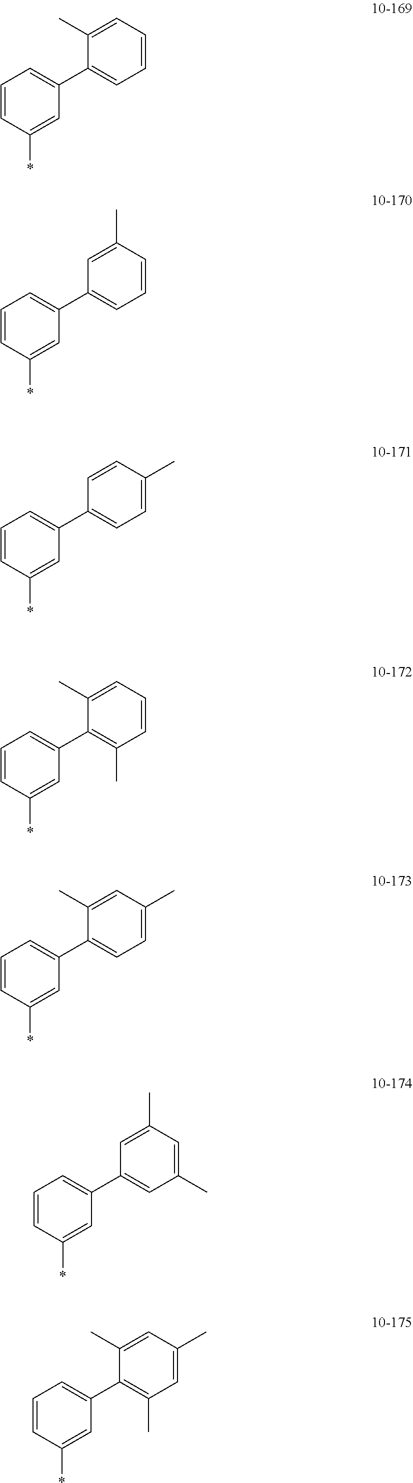

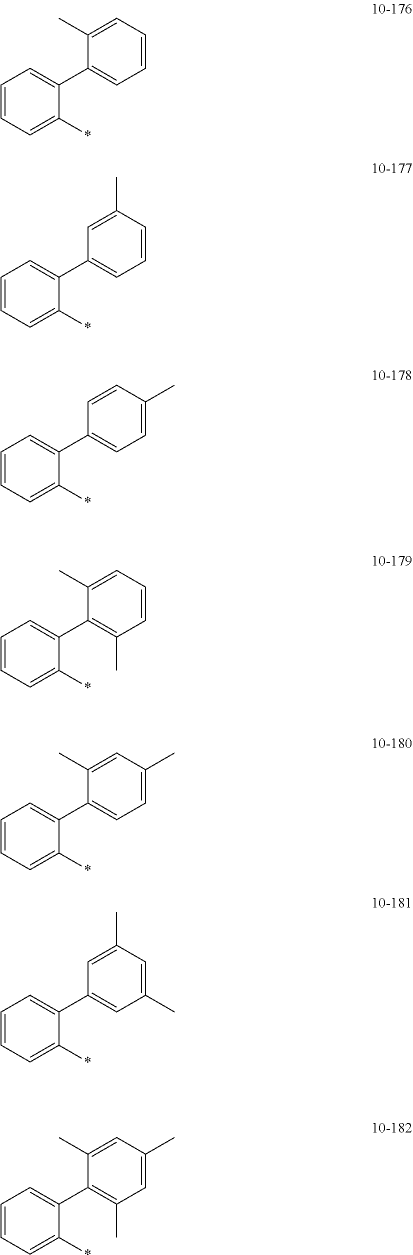

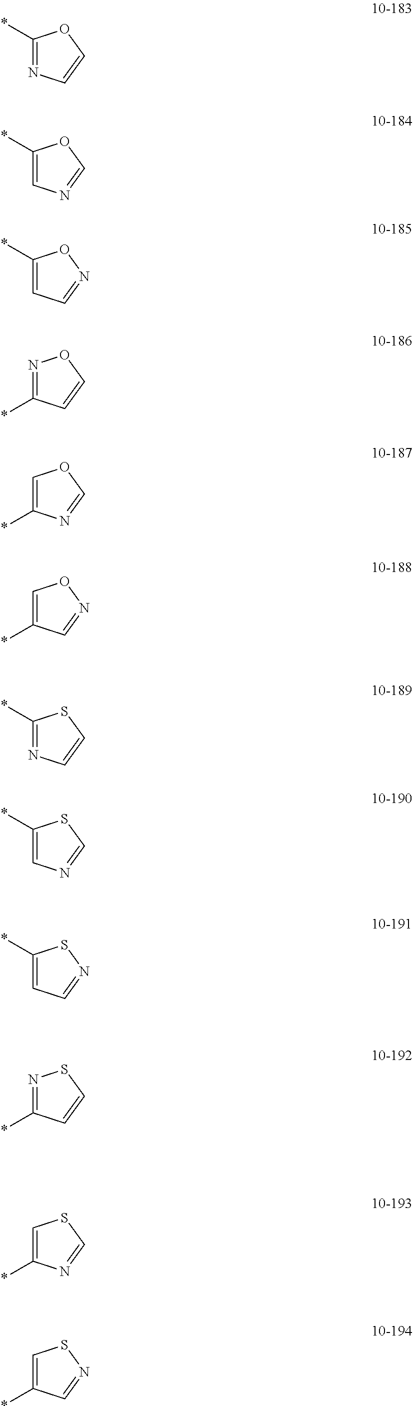

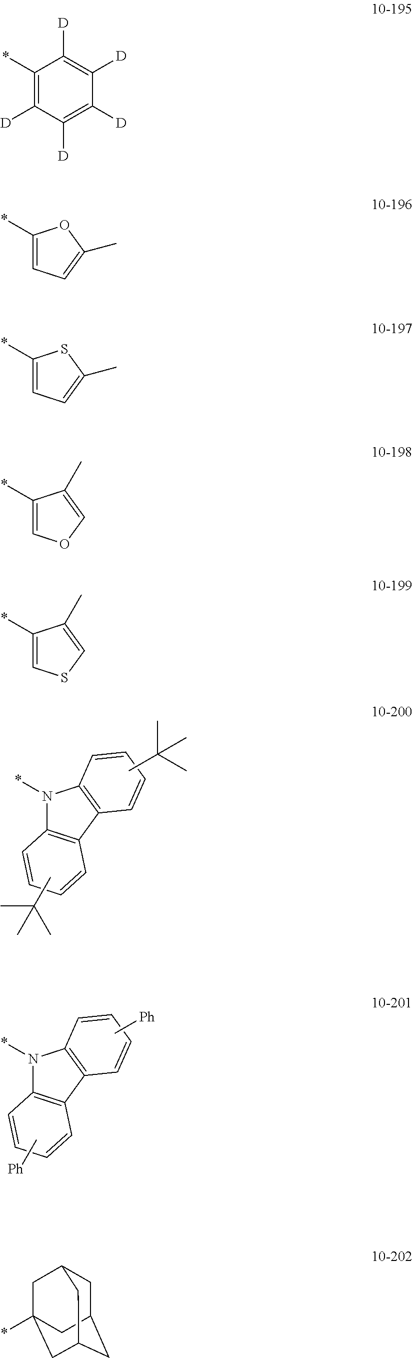

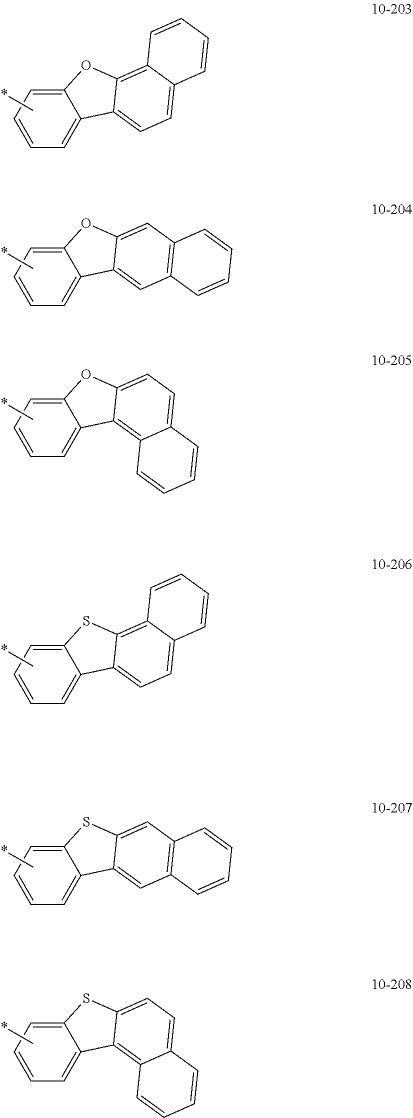

In Formulae 9-1 to 9-19 and 10-1 to 10-208, * indicates a binding site to a neighboring atom, Ph may be a phenyl group, and TMS may be a trimethylsilyl group.

In one or more embodiments, R₁₀, R₂₀, R₃₀, and R₄₀ may each be hydrogen.

In one or more embodiments, the heterocyclic compound represented by Formula 1 may be represented by one of Formulae 10-1 to 10-6:

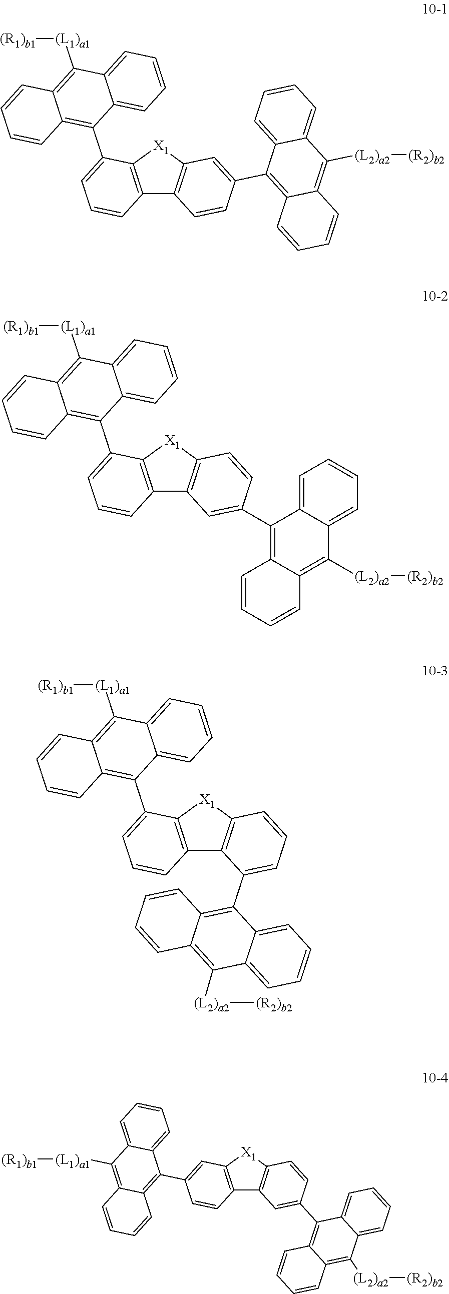

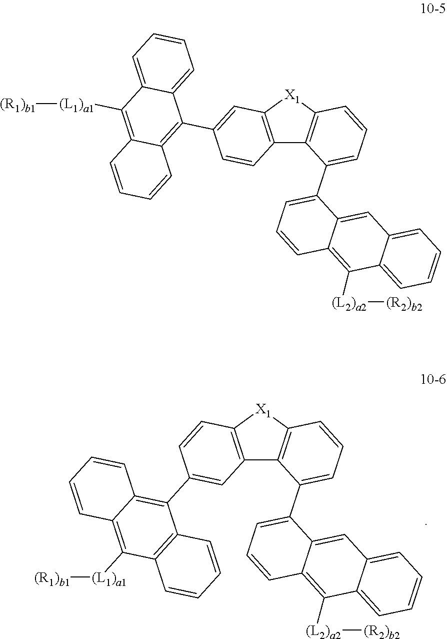

In Formulae 10-1 to 10-6,
X₁, L₁, L₂, a1, a2, R₁, R₂, b1, and b2 are the same as described above.

In one or more embodiments, the heterocyclic compound may be one of Compounds 1 to 2120:

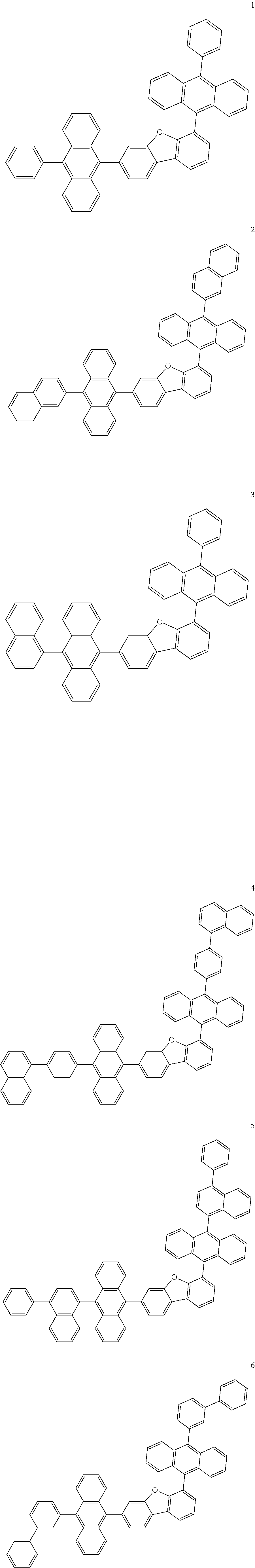

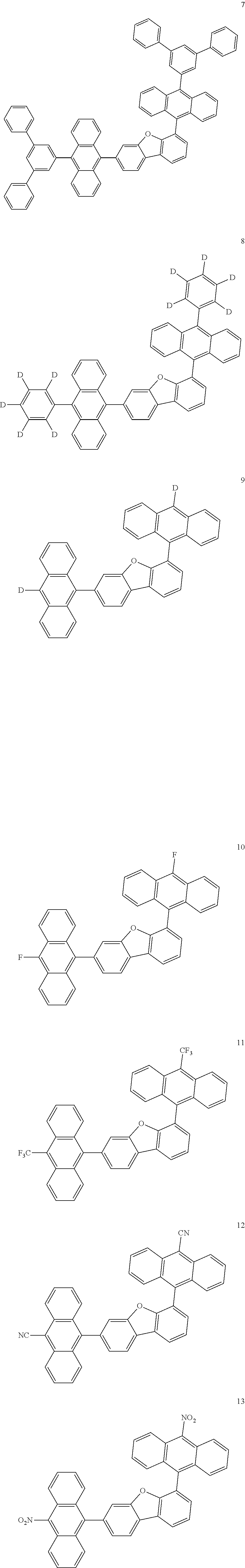

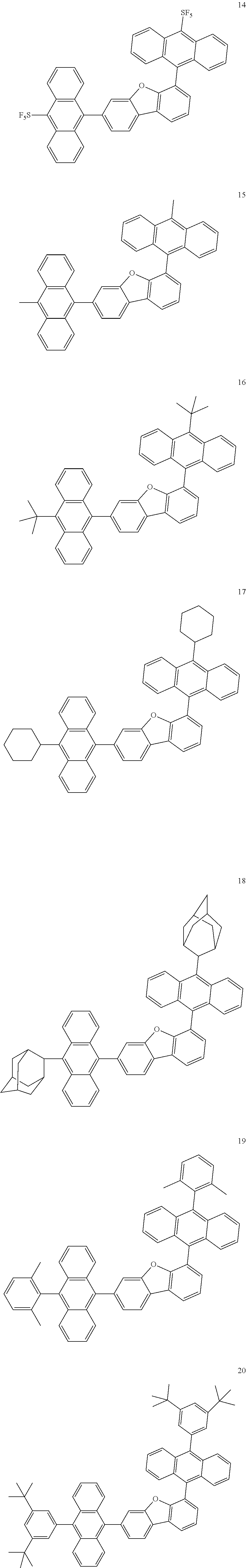

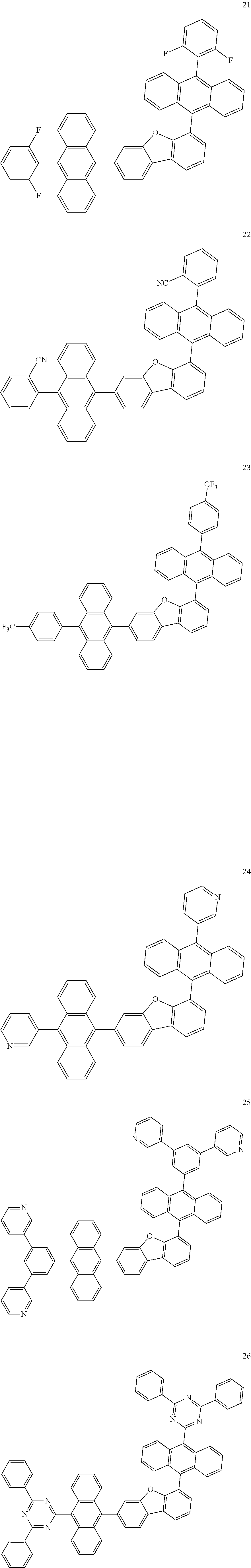

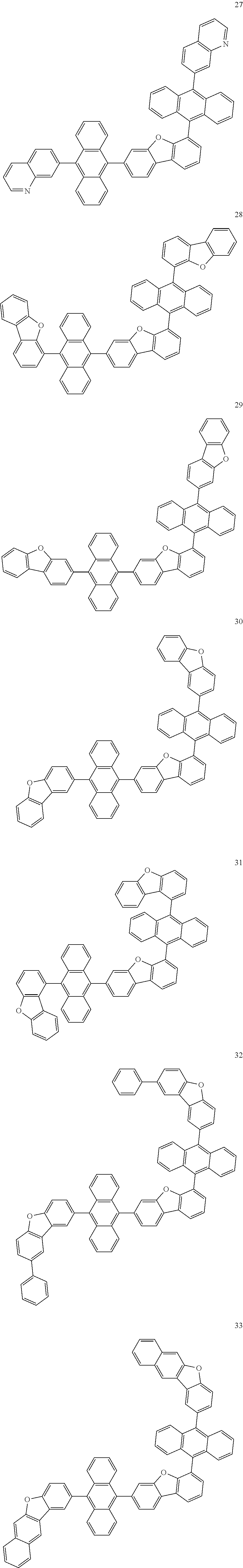

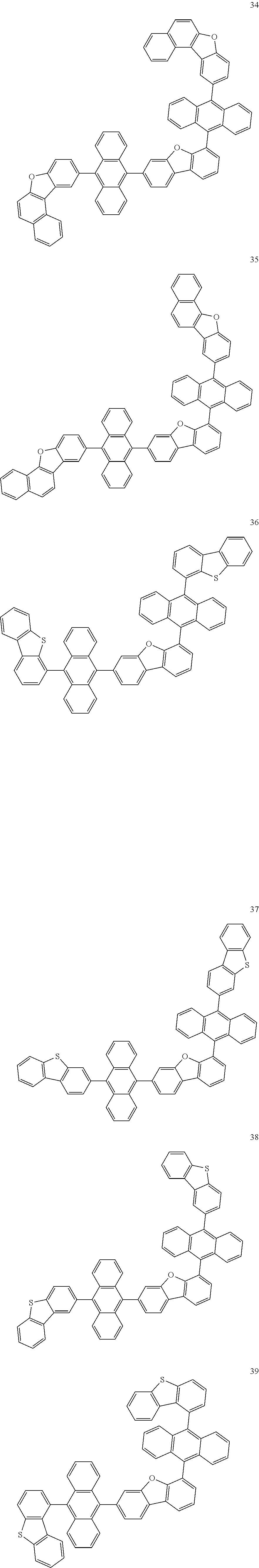

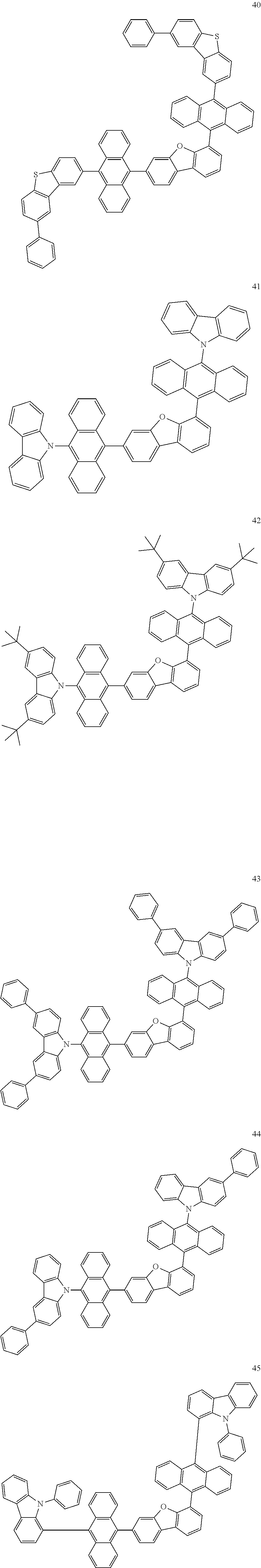

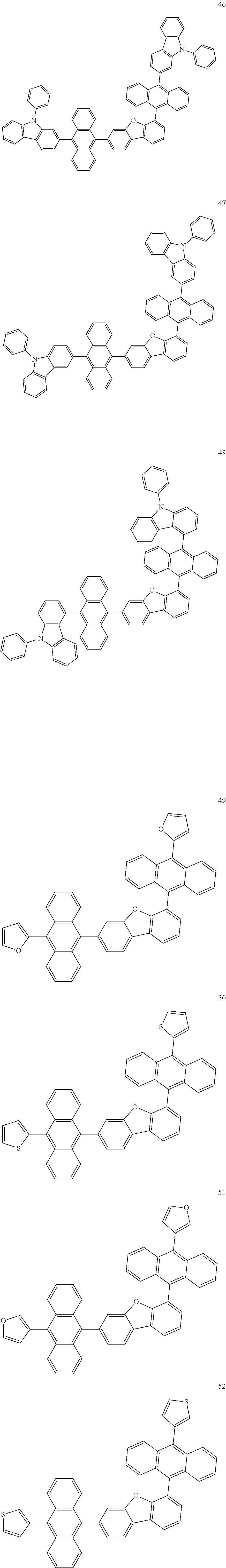

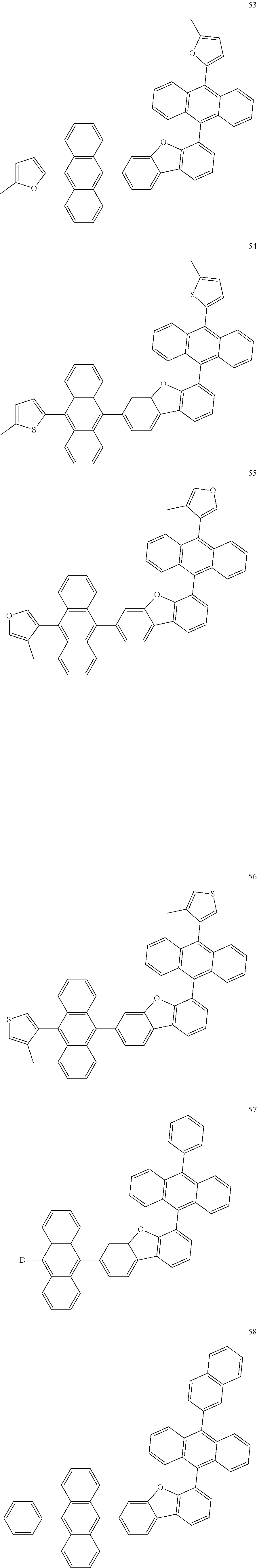

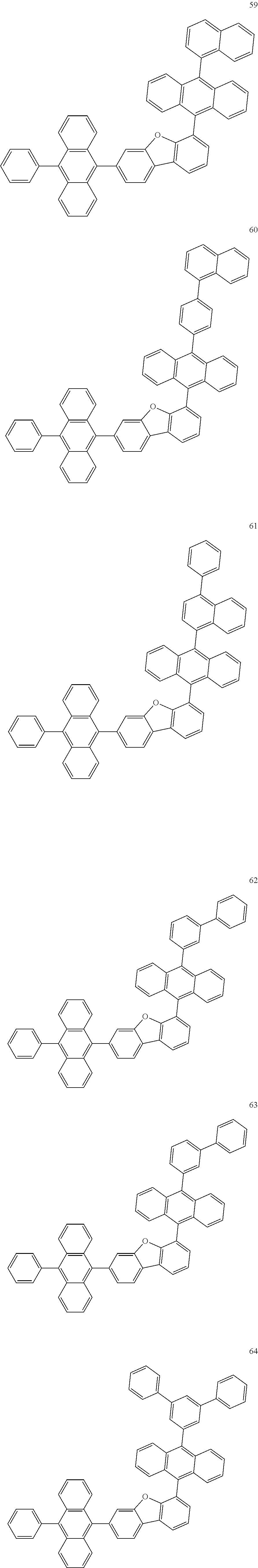

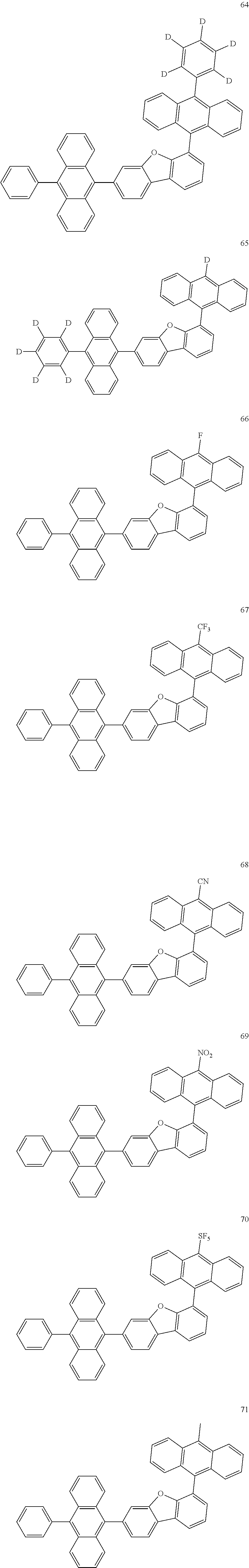

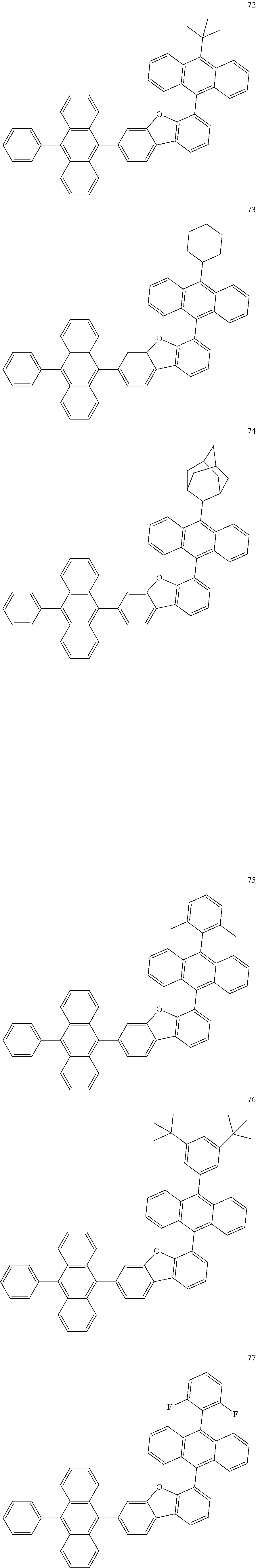

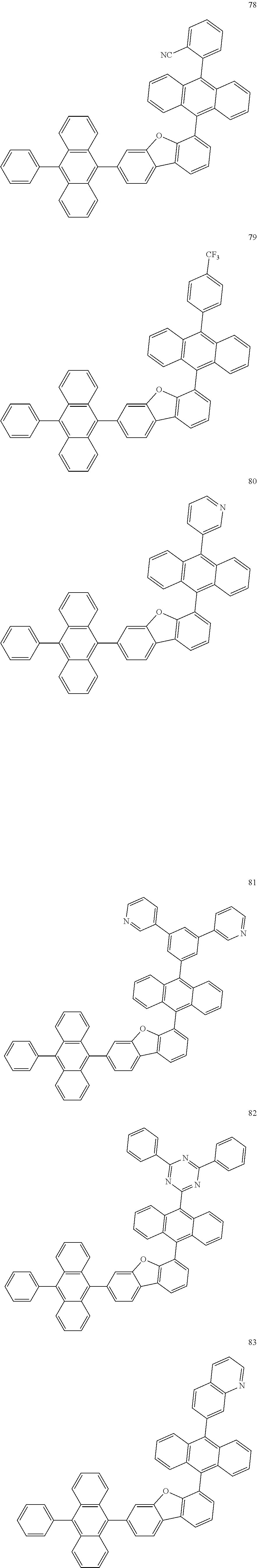

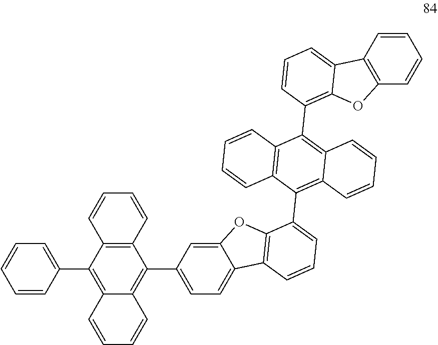 [SMILES: c1ccc(-c2c3ccccc3c(-c3ccc4c(c3)oc3c(-c5c6ccccc6c(-c6cccc7c6oc6ccccc67)c6ccccc56)cccc34)c3ccccc23)cc1]

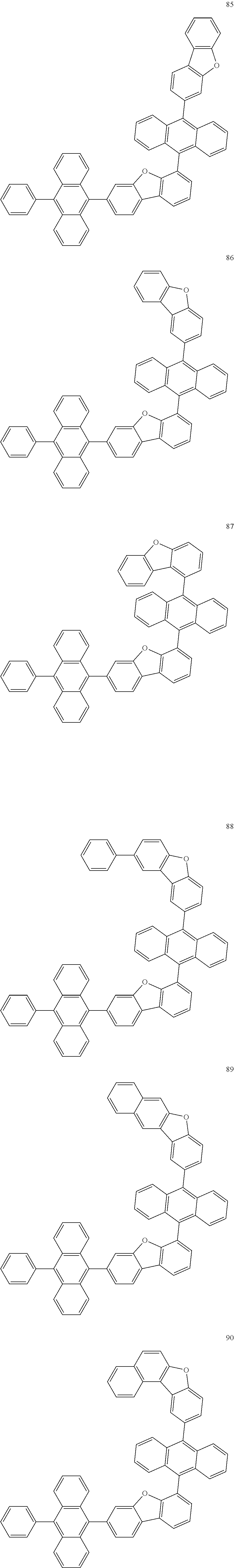

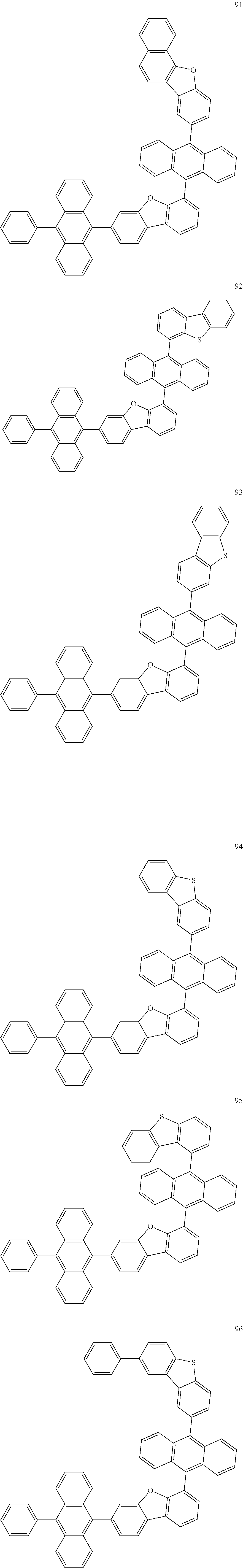

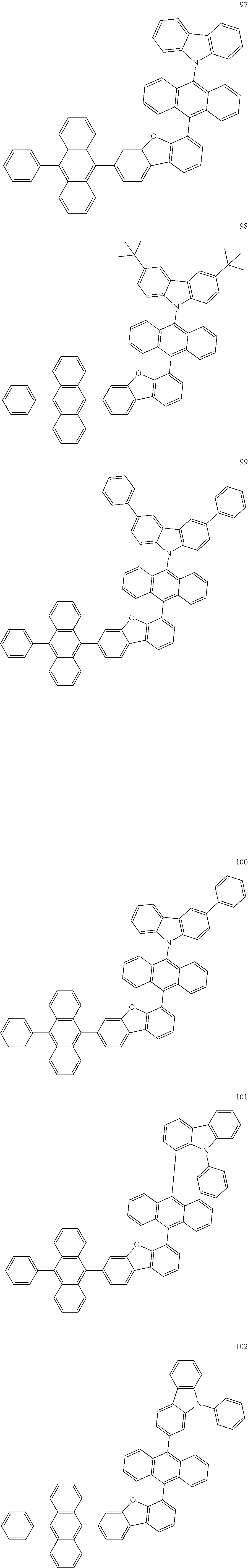

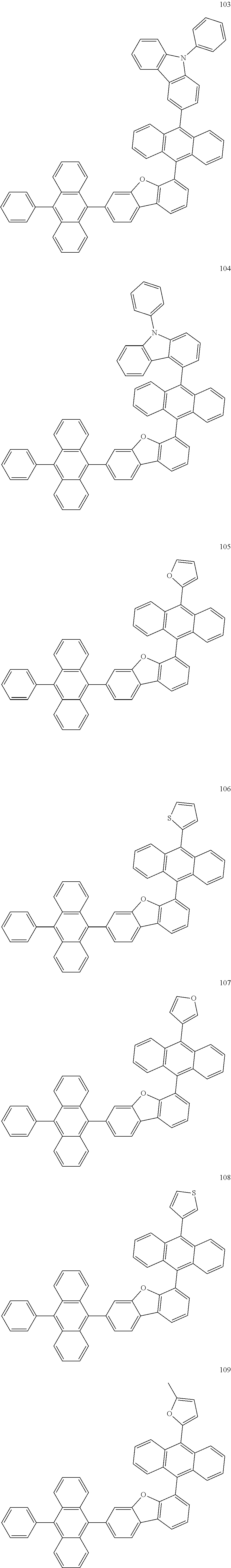

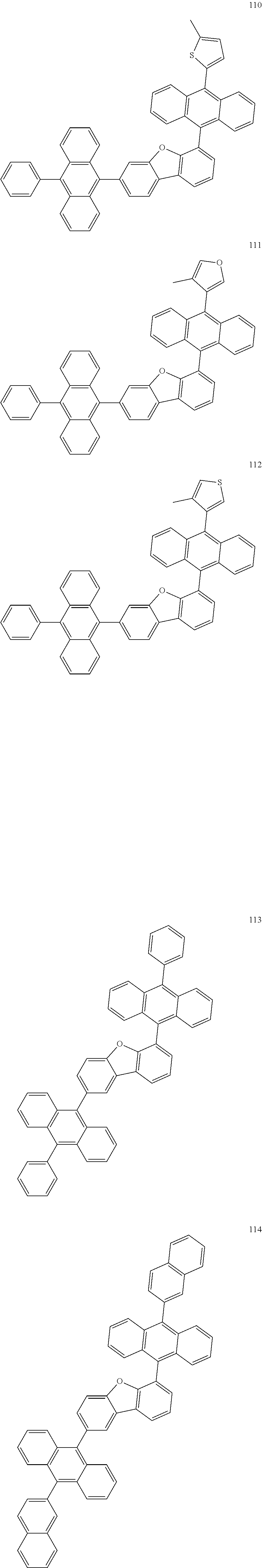 [SMILES: Cc1ccc(-c2c3ccccc3c(-c3cccc4c3oc3cc(-c5c6ccccc6c(-c6ccccc6)c6ccccc56)ccc34)c3ccccc23)s1 ; Cc1cocc1-c1c2ccccc2c(-c2cccc3c2oc2cc(-c4c5ccccc5c(-c5ccccc5)c5ccccc45)ccc23)c2ccccc12 ; Cc1cscc1-c1c2ccccc2c(-c2cccc3c2oc2cc(-c4c5ccccc5c(-c5ccccc5)c5ccccc45)ccc23)c2ccccc12 ; c1ccc(-c2c3ccccc3c(-c3ccc4oc5c(-c6c7ccccc7c(-c7ccccc7)c7ccccc67)cccc5c4c3)c3ccccc23)cc1 ; c1ccc2cc(-c3c4ccccc4c(-c4ccc5oc6c(-c7c8ccccc8c(-c8ccc9ccccc9c8)c8ccccc78)cccc6c5c4)c4ccccc34)ccc2c1]

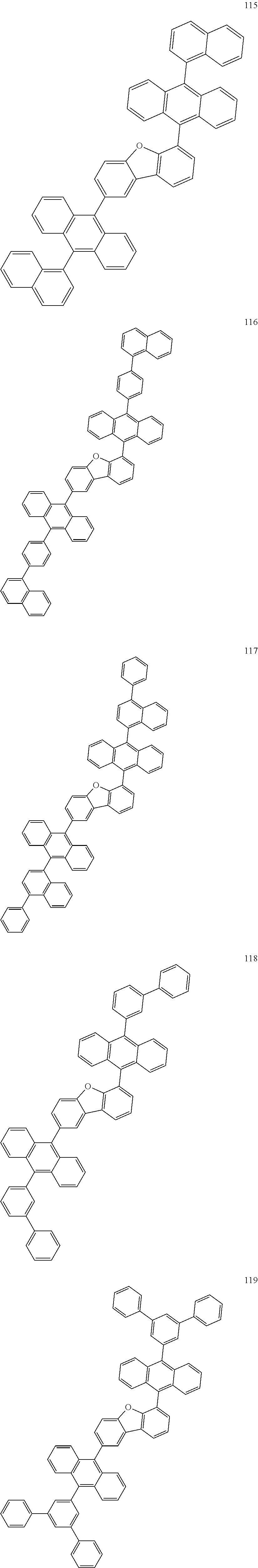

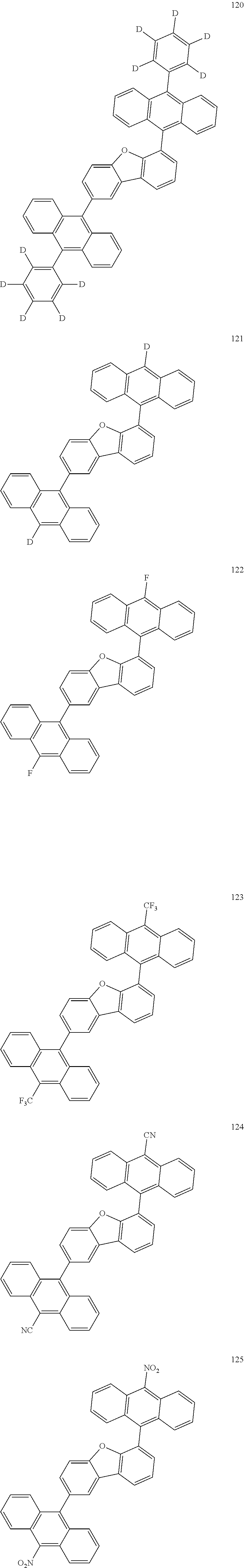

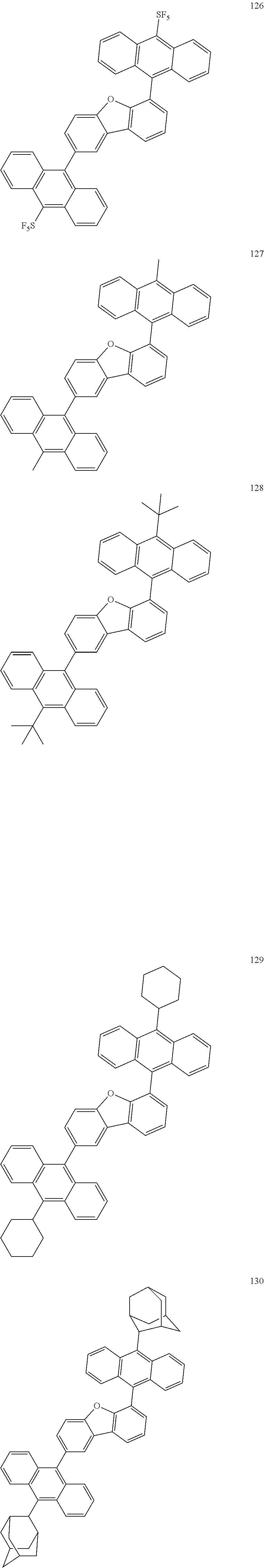

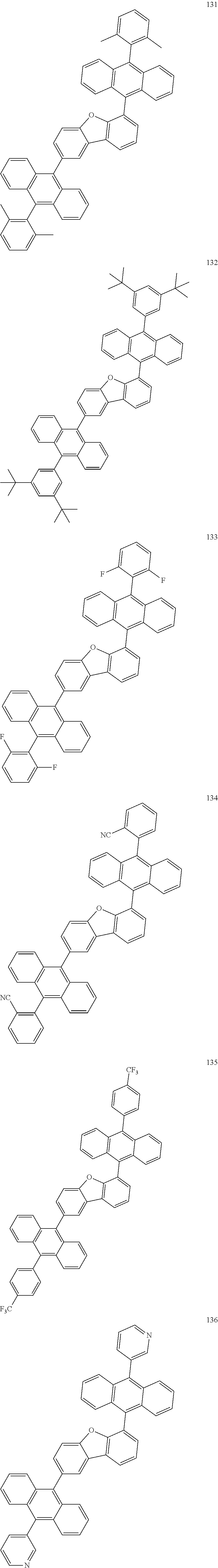

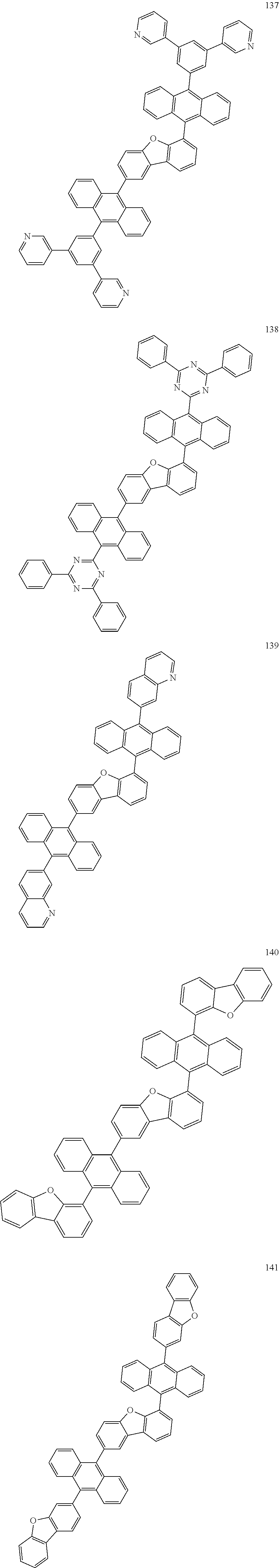

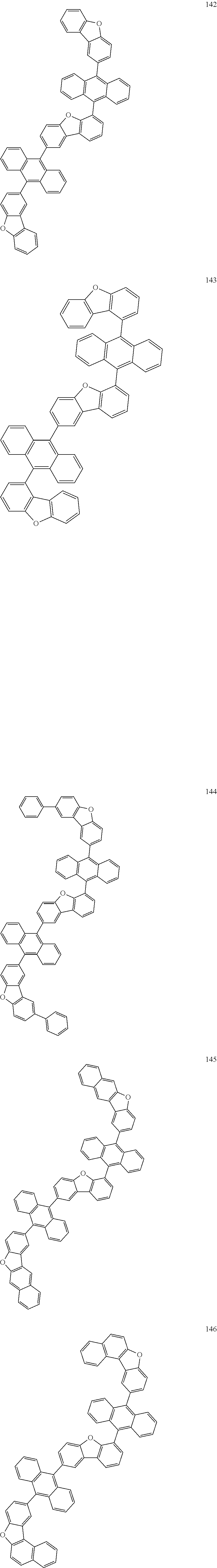

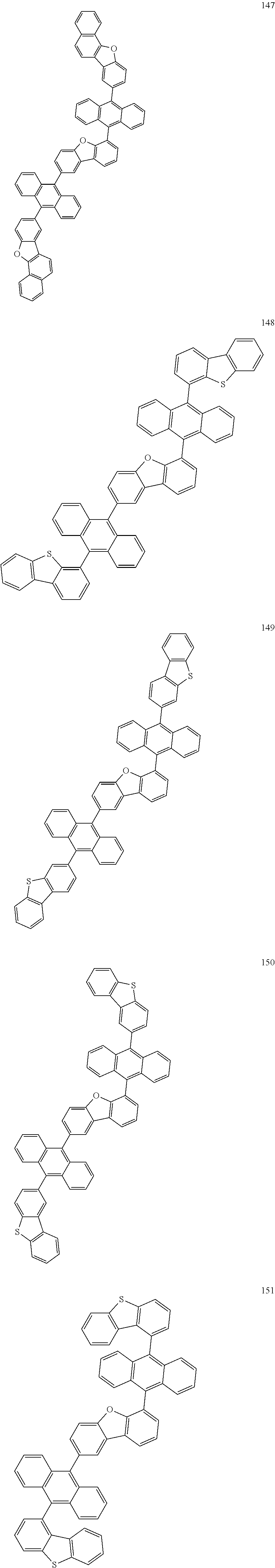

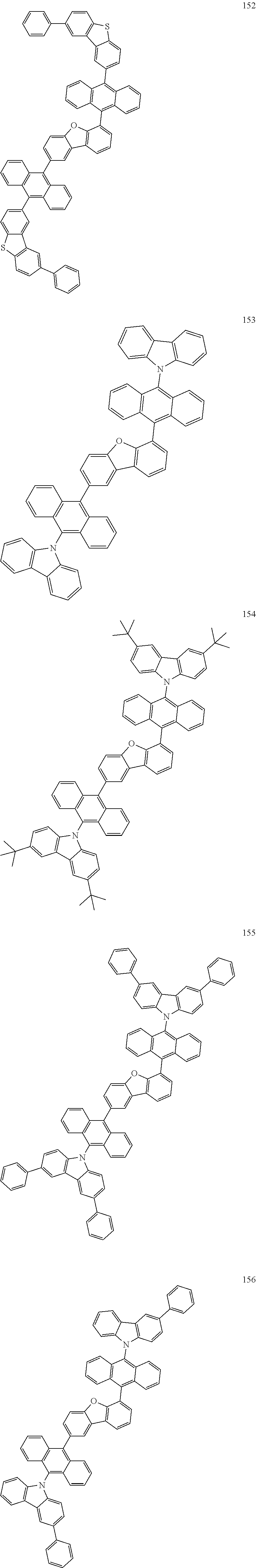

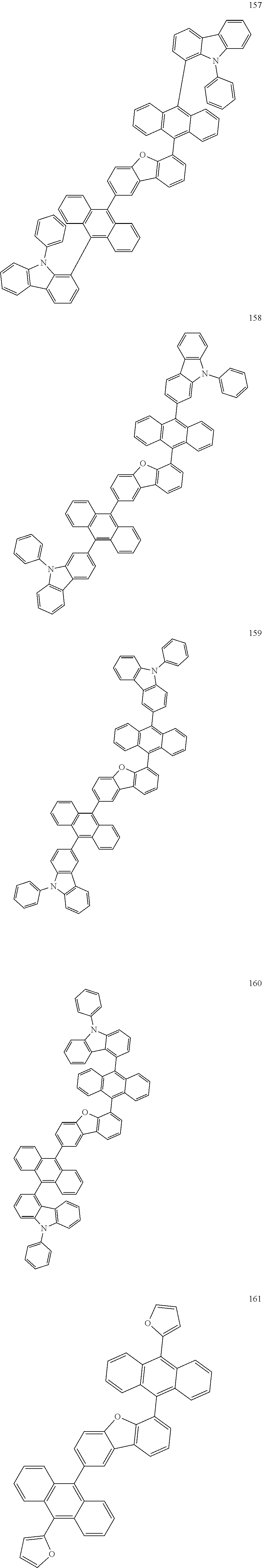

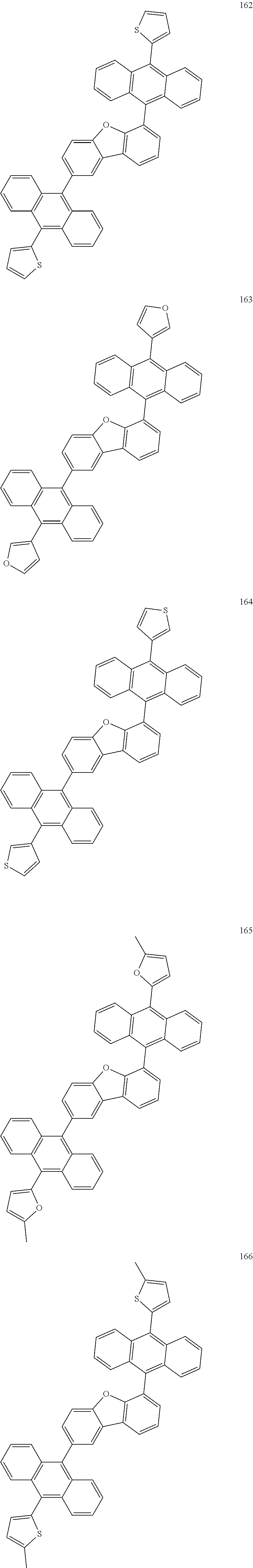 [SMILES: c1csc(-c2c3ccccc3c(-c3ccc4oc5c(-c6c7ccccc7c(-c7cccs7)c7ccccc67)cccc5c4c3)c3ccccc23)c1 ; c1ccc2c(-c3ccc4oc5c(-c6c7ccccc7c(-c7ccoc7)c7ccccc67)cccc5c4c3)c3ccccc3c(-c3ccoc3)c2c1 ; c1ccc2c(-c3ccc4oc5c(-c6c7ccccc7c(-c7ccsc7)c7ccccc67)cccc5c4c3)c3ccccc3c(-c3ccsc3)c2c1 ; Cc1ccc(-c2c3ccccc3c(-c3ccc4oc5c(-c6c7ccccc7c(-c7ccc(C)o7)c7ccccc67)cccc5c4c3)c3ccccc23)o1 ; Cc1ccc(-c2c3ccccc3c(-c3ccc4oc5c(-c6c7ccccc7c(-c7ccc(C)s7)c7ccccc67)cccc5c4c3)c3ccccc23)s1]

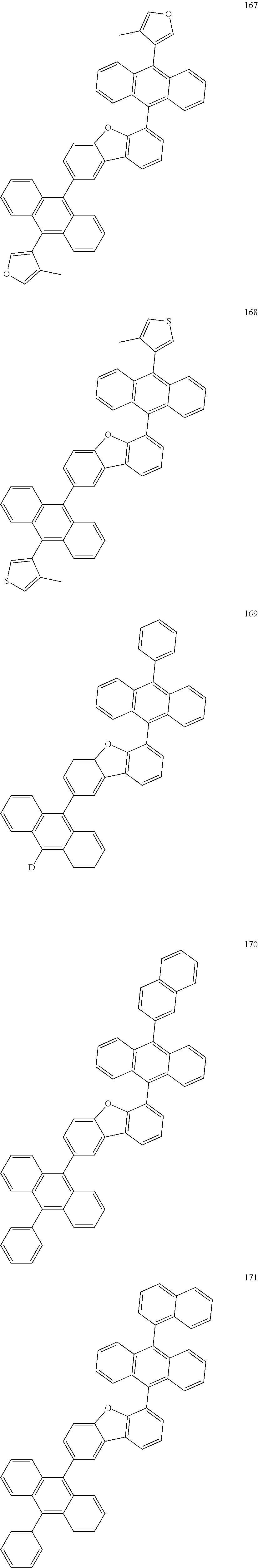 [SMILES: Cc1cocc1-c1c2ccccc2c(-c2ccc3oc4c(-c5c6ccccc6c(-c6cocc6C)c6ccccc56)cccc4c3c2)c2ccccc12 ; Cc1cscc1-c1c2ccccc2c(-c2ccc3oc4c(-c5c6ccccc6c(-c6cscc6C)c6ccccc56)cccc4c3c2)c2ccccc12 ; [2H]c1c2ccccc2c(-c2ccc3oc4c(-c5c6ccccc6c(-c6ccccc6)c6ccccc56)cccc4c3c2)c2ccccc12 ; c1ccc(-c2c3ccccc3c(-c3ccc4oc5c(-c6c7ccccc7c(-c7ccc8ccccc8c7)c7ccccc67)cccc5c4c3)c3ccccc23)cc1 ; c1ccc(-c2c3ccccc3c(-c3ccc4oc5c(-c6c7ccccc7c(-c7cccc8ccccc78)c7ccccc67)cccc5c4c3)c3ccccc23)cc1]

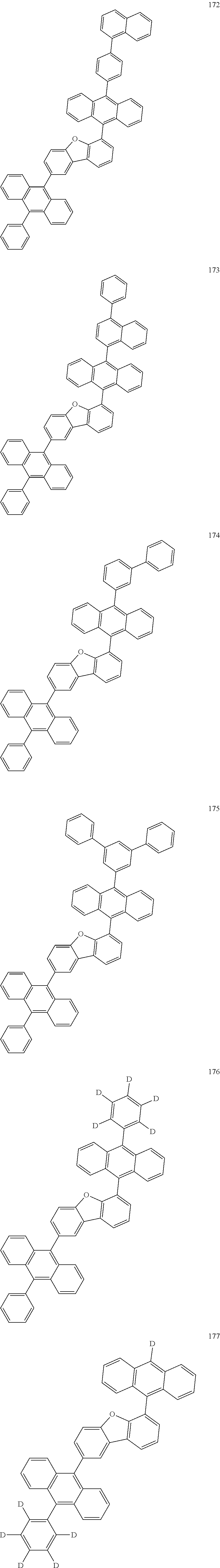

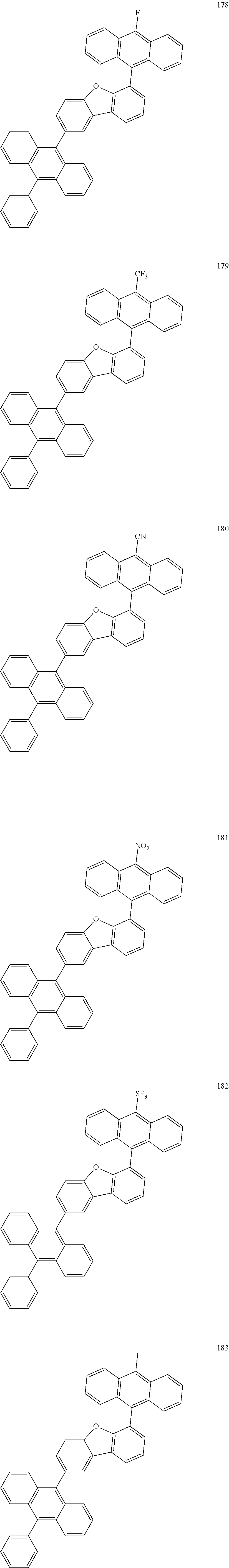

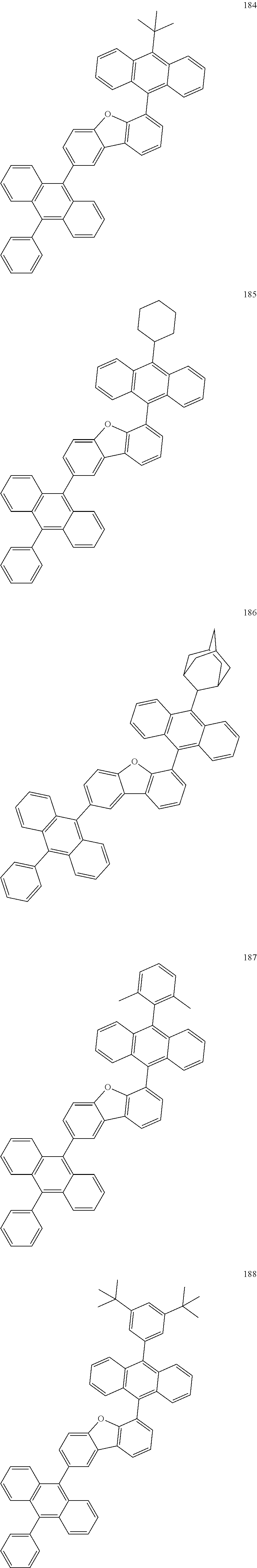 [SMILES: CC(C)(C)c1c2ccccc2c(-c2cccc3c2oc2ccc(-c4c5ccccc5c(-c5ccccc5)c5ccccc45)cc23)c2ccccc12 ; c1ccc(-c2c3ccccc3c(-c3ccc4oc5c(-c6c7ccccc7c(C7CCCCC7)c7ccccc67)cccc5c4c3)c3ccccc23)cc1 ; c1ccc(-c2c3ccccc3c(-c3ccc4oc5c(-c6c7ccccc7c(C7C8CC9CC(C8)CC7C9)c7ccccc67)cccc5c4c3)c3ccccc23)cc1 ; Cc1cccc(C)c1-c1c2ccccc2c(-c2cccc3c2oc2ccc(-c4c5ccccc5c(-c5ccccc5)c5ccccc45)cc23)c2ccccc12 ; CC(C)(C)c1cc(-c2c3ccccc3c(-c3cccc4c3oc3ccc(-c5c6ccccc6c(-c6ccccc6)c6ccccc56)cc34)c3ccccc23)cc(C(C)(C)C)c1]

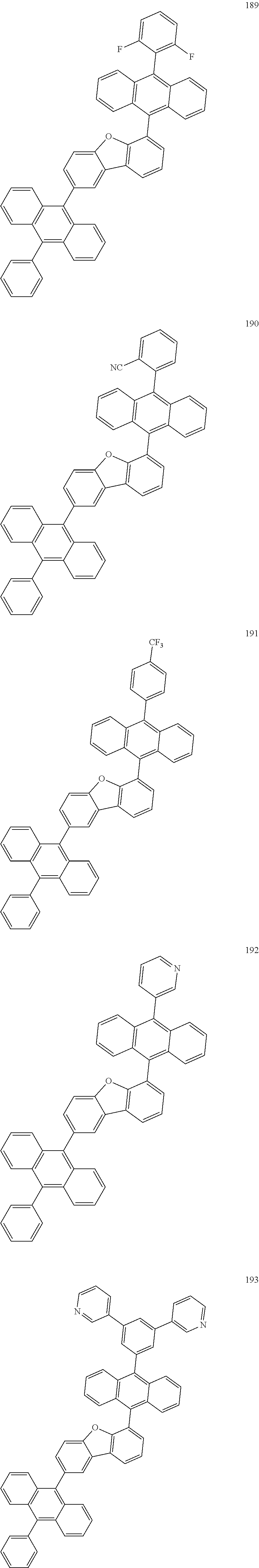 [SMILES: Fc1cccc(F)c1-c1c2ccccc2c(-c2cccc3c2oc2ccc(-c4c5ccccc5c(-c5ccccc5)c5ccccc45)cc23)c2ccccc12 ; N#Cc1ccccc1-c1c2ccccc2c(-c2cccc3c2oc2ccc(-c4c5ccccc5c(-c5ccccc5)c5ccccc45)cc23)c2ccccc12 ; FC(F)(F)c1ccc(-c2c3ccccc3c(-c3cccc4c3oc3ccc(-c5c6ccccc6c(-c6ccccc6)c6ccccc56)cc34)c3ccccc23)cc1 ; c1ccc(-c2c3ccccc3c(-c3ccc4oc5c(-c6c7ccccc7c(-c7cccnc7)c7ccccc67)cccc5c4c3)c3ccccc23)cc1 ; c1ccc(-c2c3ccccc3c(-c3ccc4oc5c(-c6c7ccccc7c(-c7cc(-c8cccnc8)cc(-c8cccnc8)c7)c7ccccc67)cccc5c4c3)c3ccccc23)cc1]

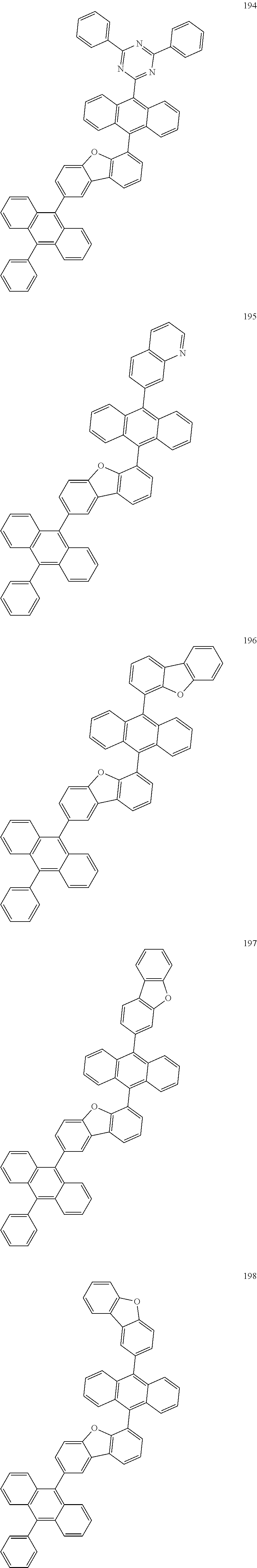

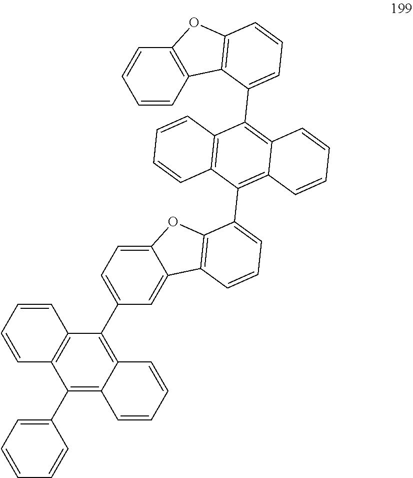 [SMILES: c1ccc(-c2c3ccccc3c(-c3ccc4oc5c(-c6c7ccccc7c(-c7cccc8oc9ccccc9c78)c7ccccc67)cccc5c4c3)c3ccccc23)cc1]

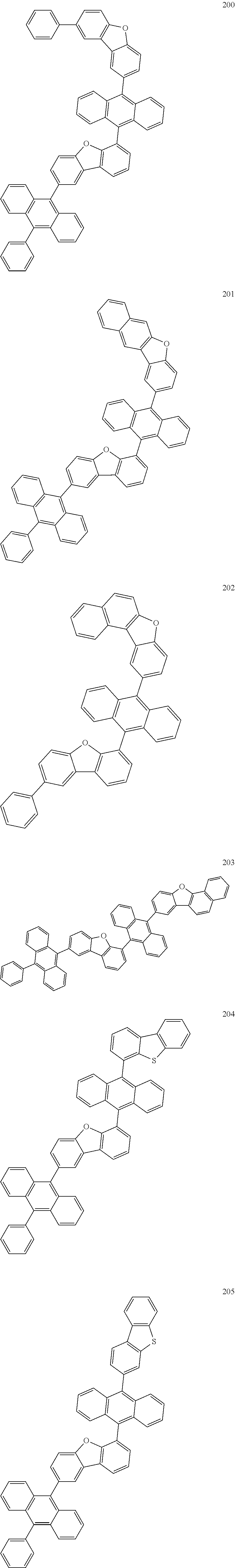

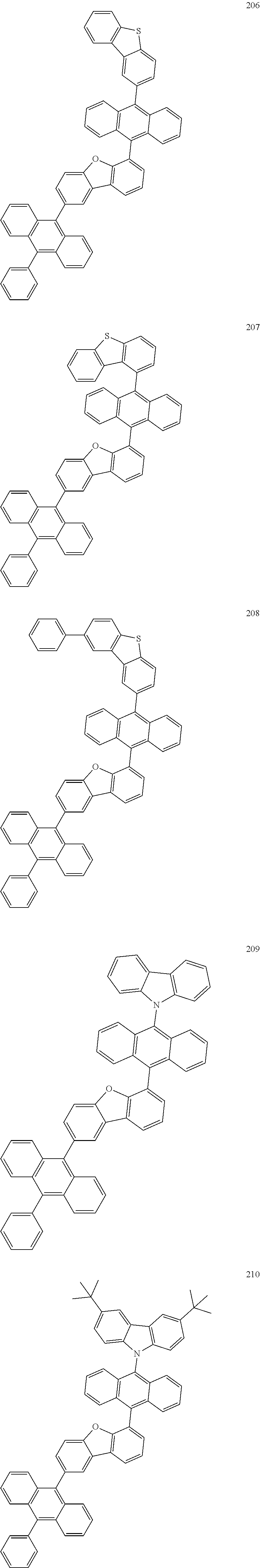

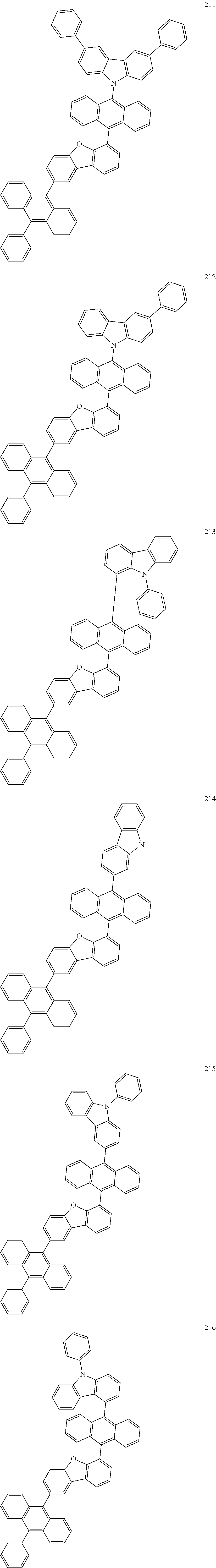

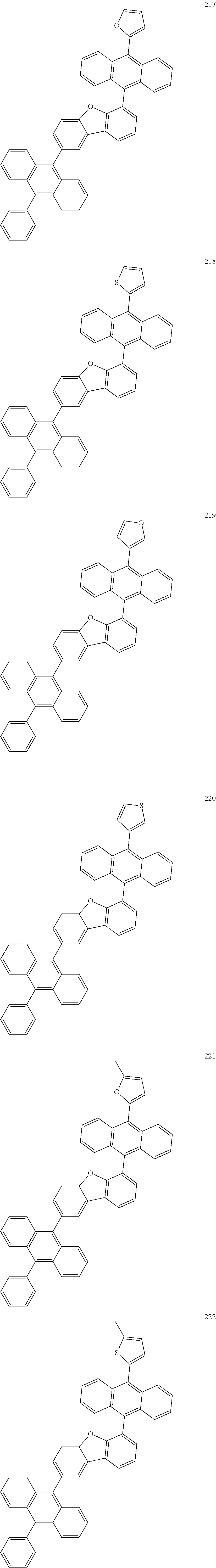

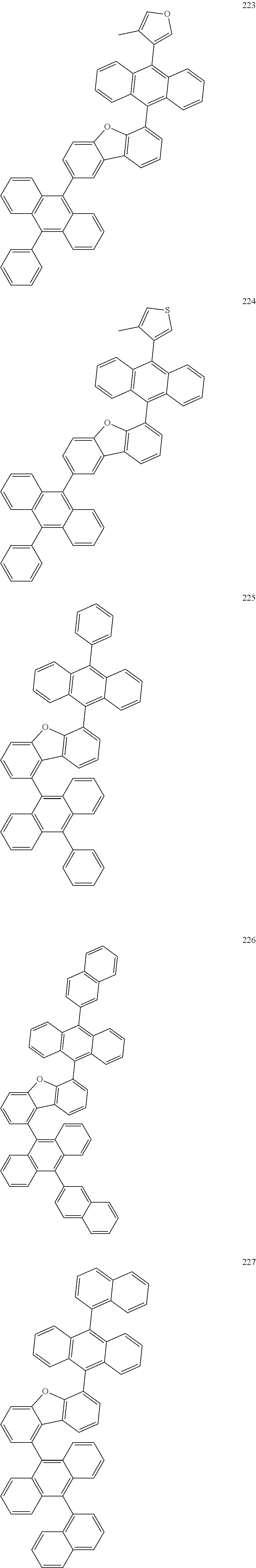 [SMILES: Cc1cocc1-c1c2ccccc2c(-c2cccc3c2oc2ccc(-c4c5ccccc5c(-c5ccccc5)c5ccccc45)cc23)c2ccccc12 ; Cc1cscc1-c1c2ccccc2c(-c2cccc3c2oc2ccc(-c4c5ccccc5c(-c5ccccc5)c5ccccc45)cc23)c2ccccc12 ; c1ccc(-c2c3ccccc3c(-c3cccc4c3oc3cccc(-c5c6ccccc6c(-c6ccccc6)c6ccccc56)c34)c3ccccc23)cc1 ; c1ccc2cc(-c3c4ccccc4c(-c4cccc5c4oc4cccc(-c6c7ccccc7c(-c7ccc8ccccc8c7)c7ccccc67)c45)c4ccccc34)ccc2c1 ; c1ccc2c(-c3c4ccccc4c(-c4cccc5c4oc4cccc(-c6c7ccccc7c(-c7cccc8ccccc78)c7ccccc67)c45)c4ccccc34)cccc2c1]

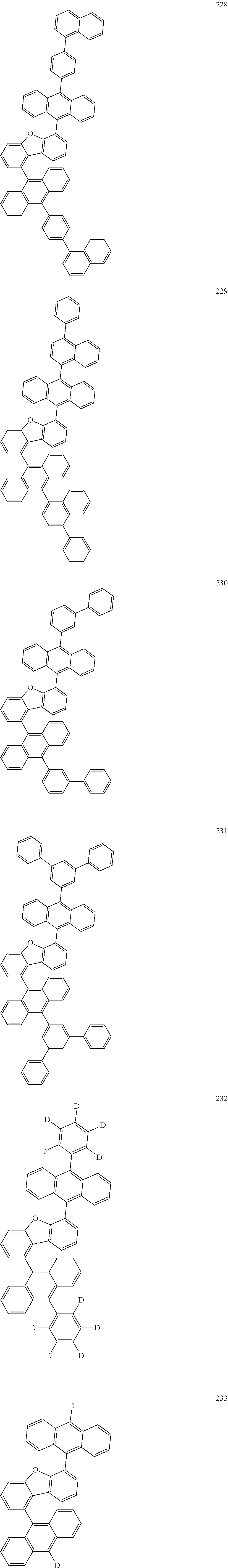

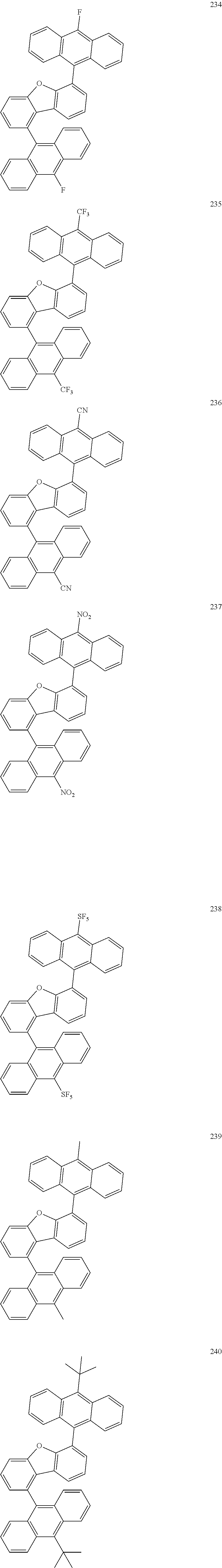

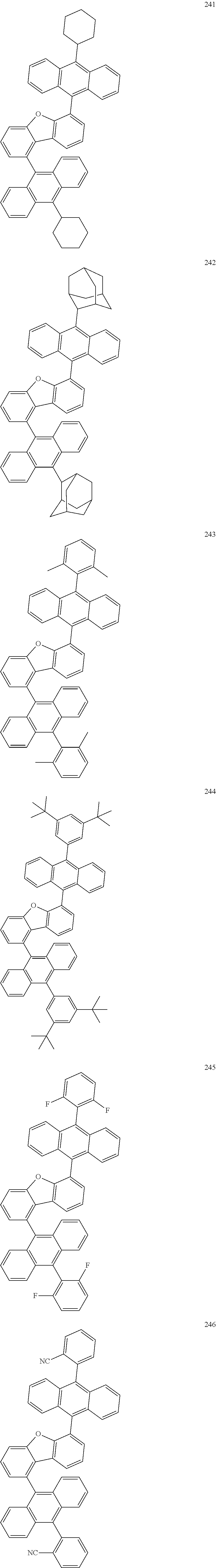

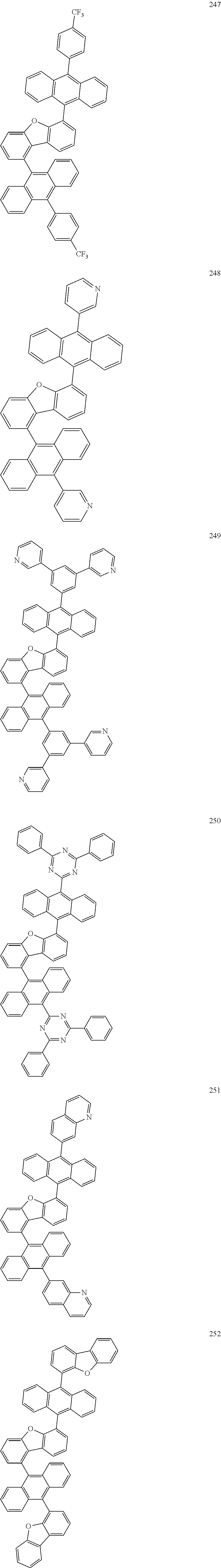

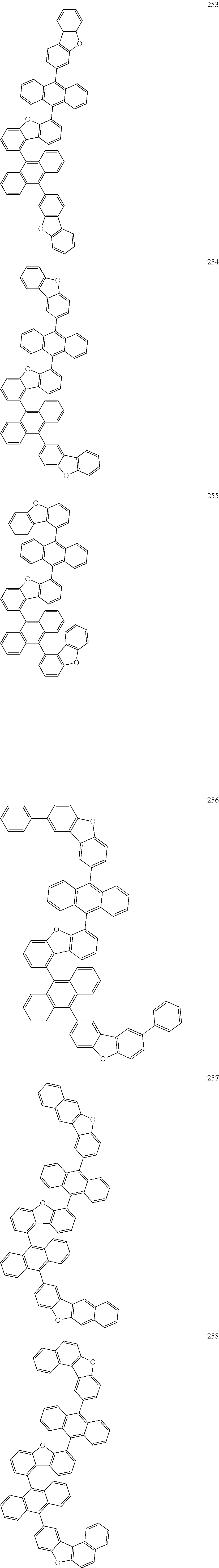

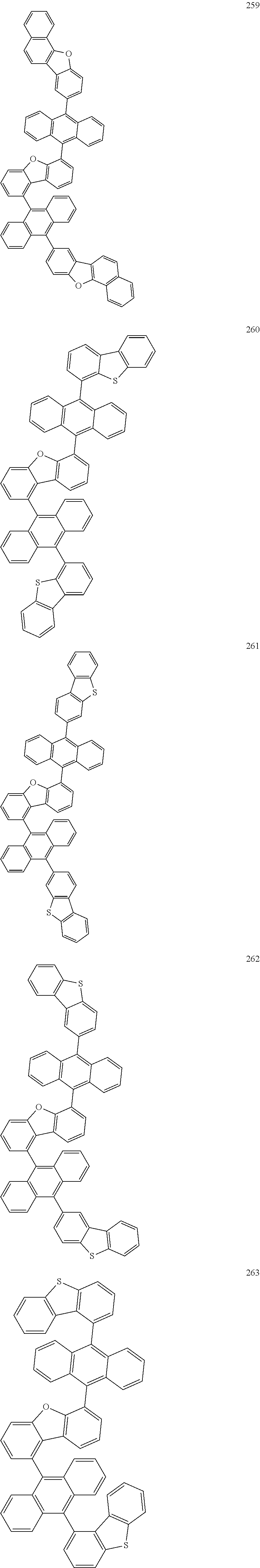

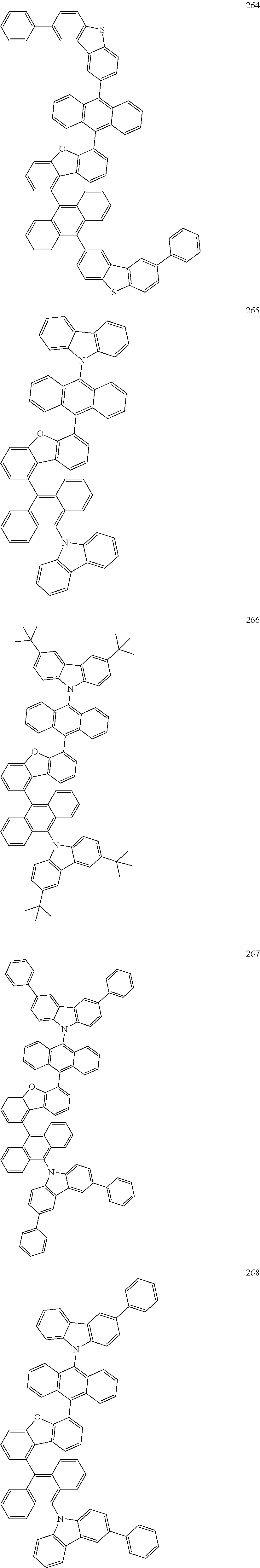

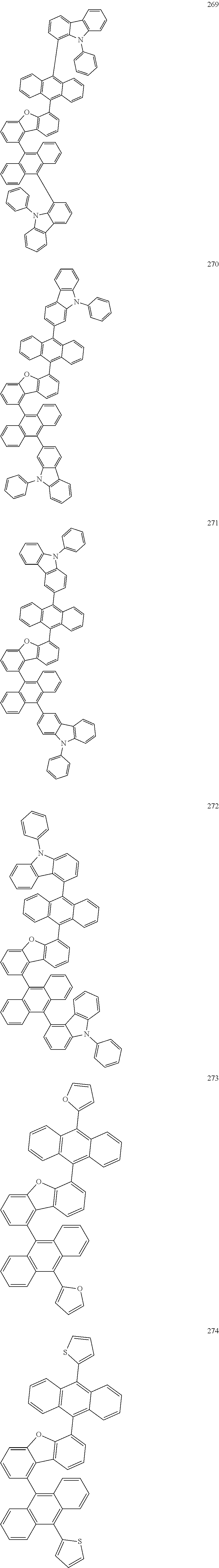

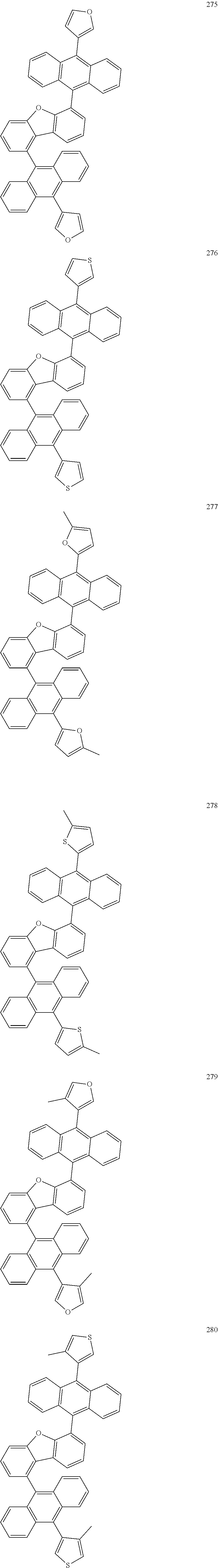

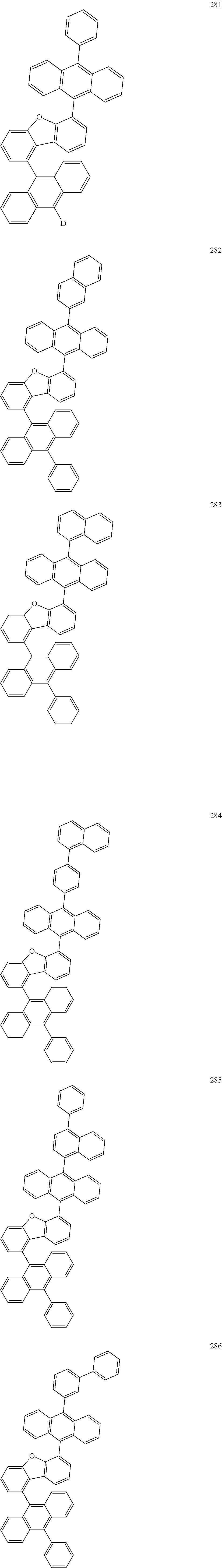

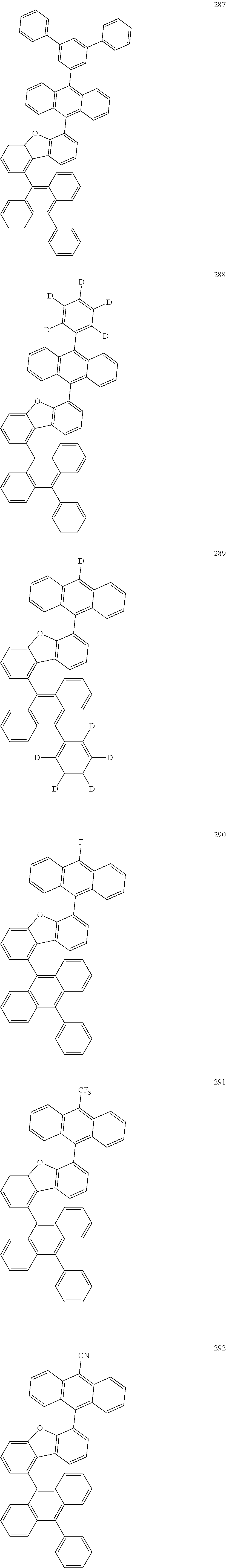

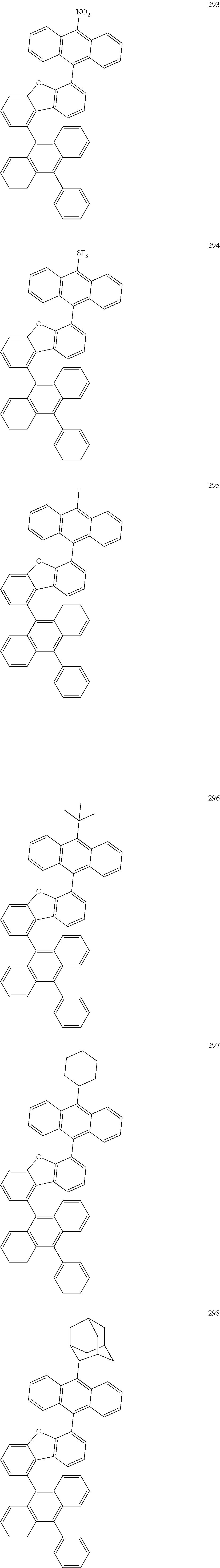

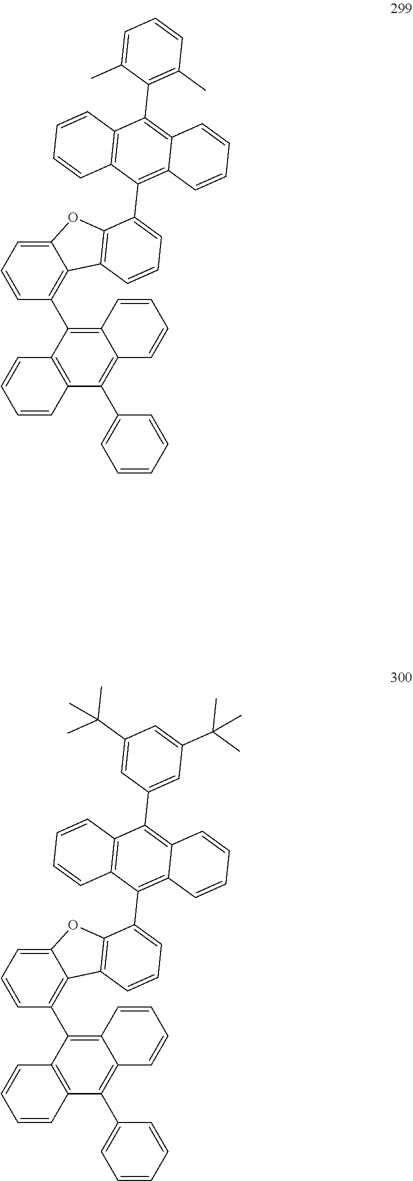 [SMILES: Cc1cccc(C)c1-c1c2ccccc2c(-c2cccc3c2oc2cccc(-c4c5ccccc5c(-c5ccccc5)c5ccccc45)c23)c2ccccc12 ; CC(C)(C)c1cc(-c2c3ccccc3c(-c3cccc4c3oc3cccc(-c5c6ccccc6c(-c6ccccc6)c6ccccc56)c34)c3ccccc23)cc(C(C)(C)C)c1]

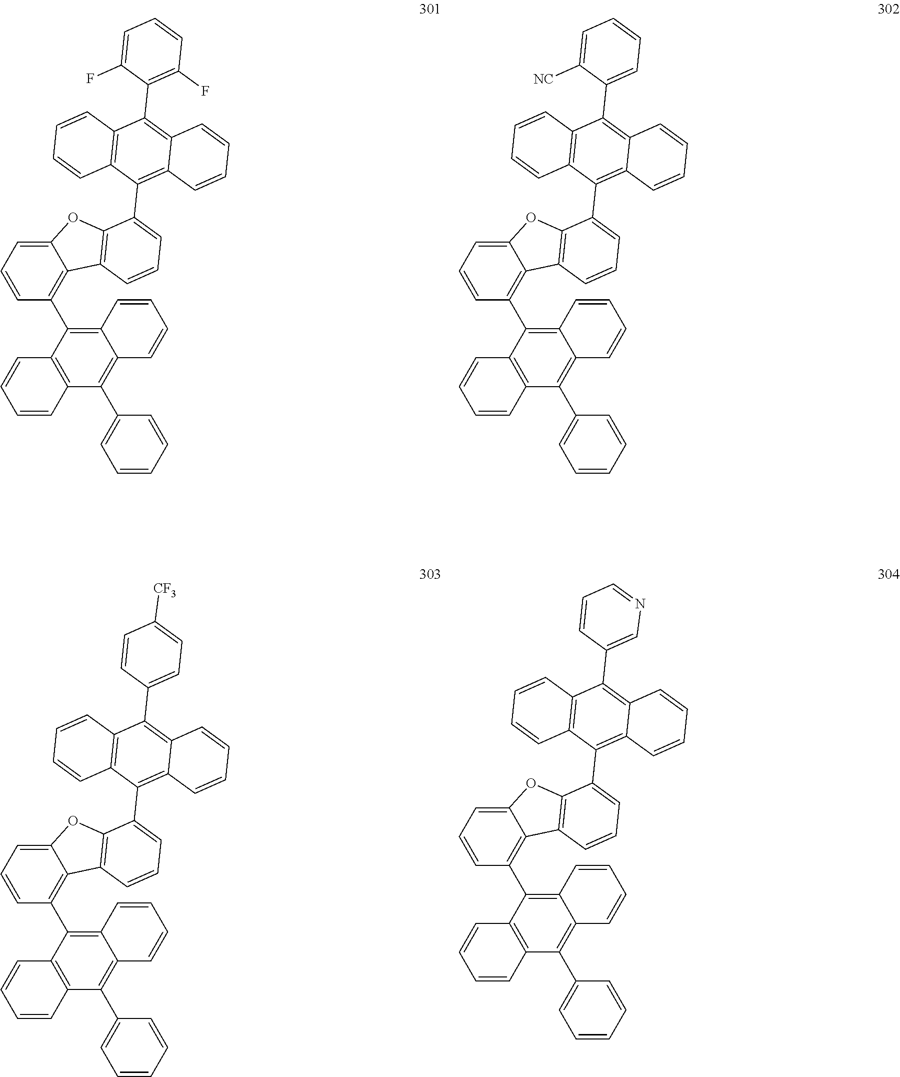 [SMILES: Fc1cccc(F)c1-c1c2ccccc2c(-c2cccc3c2oc2cccc(-c4c5ccccc5c(-c5ccccc5)c5ccccc45)c23)c2ccccc12 ; N#Cc1ccccc1-c1c2ccccc2c(-c2cccc3c2oc2cccc(-c4c5ccccc5c(-c5ccccc5)c5ccccc45)c23)c2ccccc12 ; FC(F)(F)c1ccc(-c2c3ccccc3c(-c3cccc4c3oc3cccc(-c5c6ccccc6c(-c6ccccc6)c6ccccc56)c34)c3ccccc23)cc1 ; c1ccc(-c2c3ccccc3c(-c3cccc4oc5c(-c6c7ccccc7c(-c7cccnc7)c7ccccc67)cccc5c34)c3ccccc23)cc1]

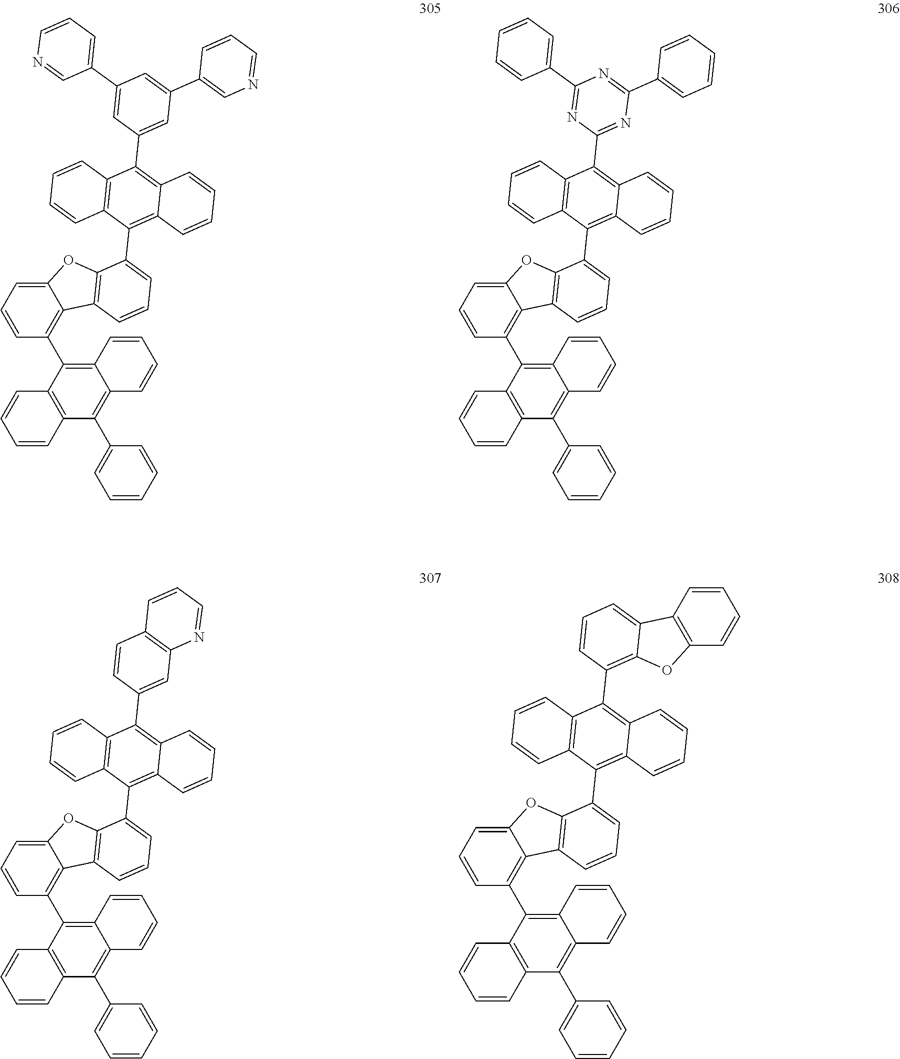 [SMILES: c1ccc(-c2c3ccccc3c(-c3cccc4oc5c(-c6c7ccccc7c(-c7cc(-c8cccnc8)cc(-c8cccnc8)c7)c7ccccc67)cccc5c34)c3ccccc23)cc1 ; c1ccc(-c2nc(-c3ccccc3)nc(-c3c4ccccc4c(-c4cccc5c4oc4cccc(-c6c7ccccc7c(-c7ccccc7)c7ccccc67)c45)c4ccccc34)n2)cc1 ; c1ccc(-c2c3ccccc3c(-c3cccc4oc5c(-c6c7ccccc7c(-c7ccc8cccnc8c7)c7ccccc67)cccc5c34)c3ccccc23)cc1 ; c1ccc(-c2c3ccccc3c(-c3cccc4oc5c(-c6c7ccccc7c(-c7cccc8c7oc7ccccc78)c7ccccc67)cccc5c34)c3ccccc23)cc1]

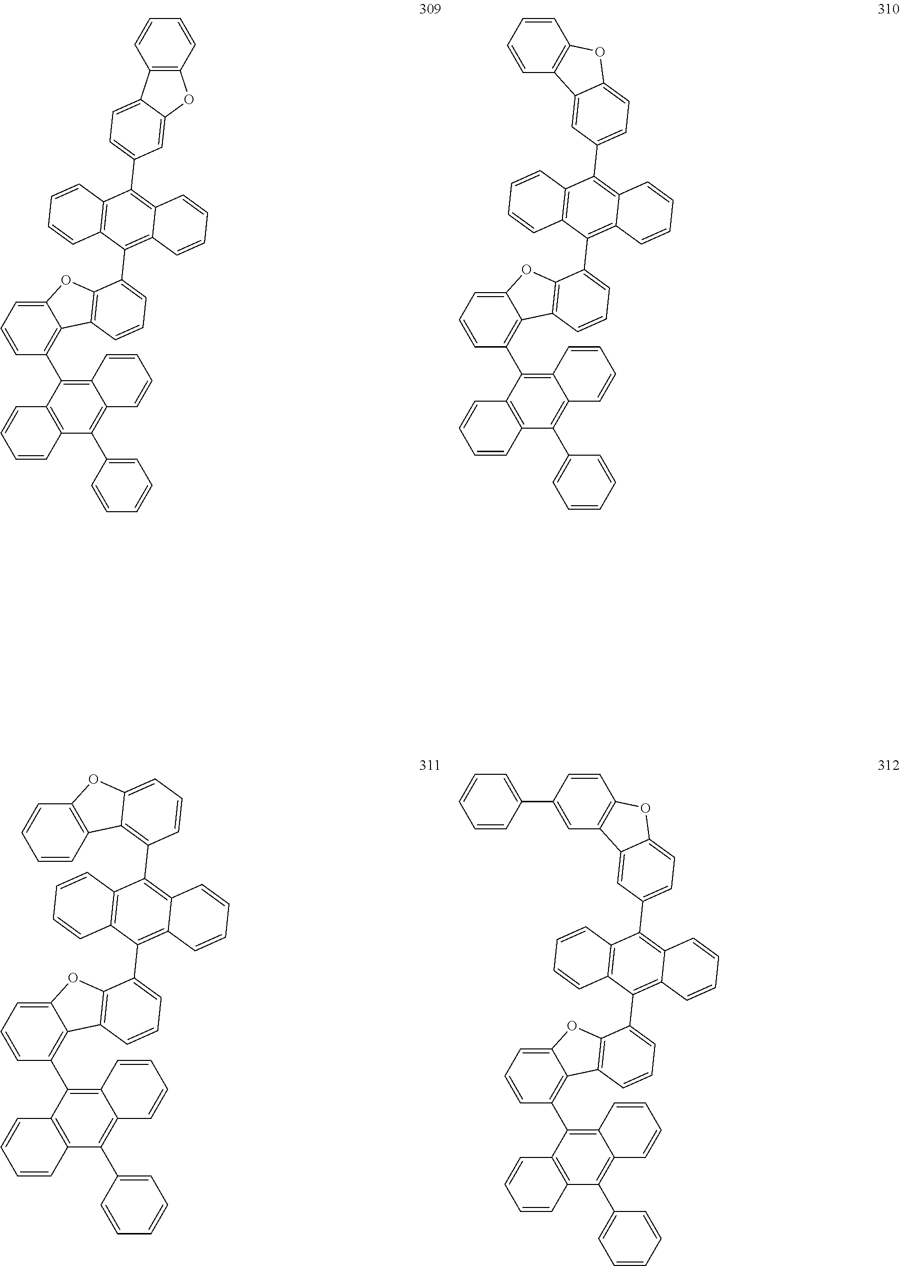 [SMILES: c1ccc(-c2c3ccccc3c(-c3cccc4oc5c(-c6c7ccccc7c(-c7ccc8c(c7)oc7ccccc78)c7ccccc67)cccc5c34)c3ccccc23)cc1 ; c1ccc(-c2c3ccccc3c(-c3cccc4oc5c(-c6c7ccccc7c(-c7ccc8oc9ccccc9c8c7)c7ccccc67)cccc5c34)c3ccccc23)cc1 ; c1ccc(-c2c3ccccc3c(-c3cccc4oc5c(-c6c7ccccc7c(-c7cccc8oc9ccccc9c78)c7ccccc67)cccc5c34)c3ccccc23)cc1 ; c1ccc(-c2ccc3oc4ccc(-c5c6ccccc6c(-c6cccc7c6oc6cccc(-c8c9ccccc9c(-c9ccccc9)c9ccccc89)c67)c6ccccc56)cc4c3c2)cc1]

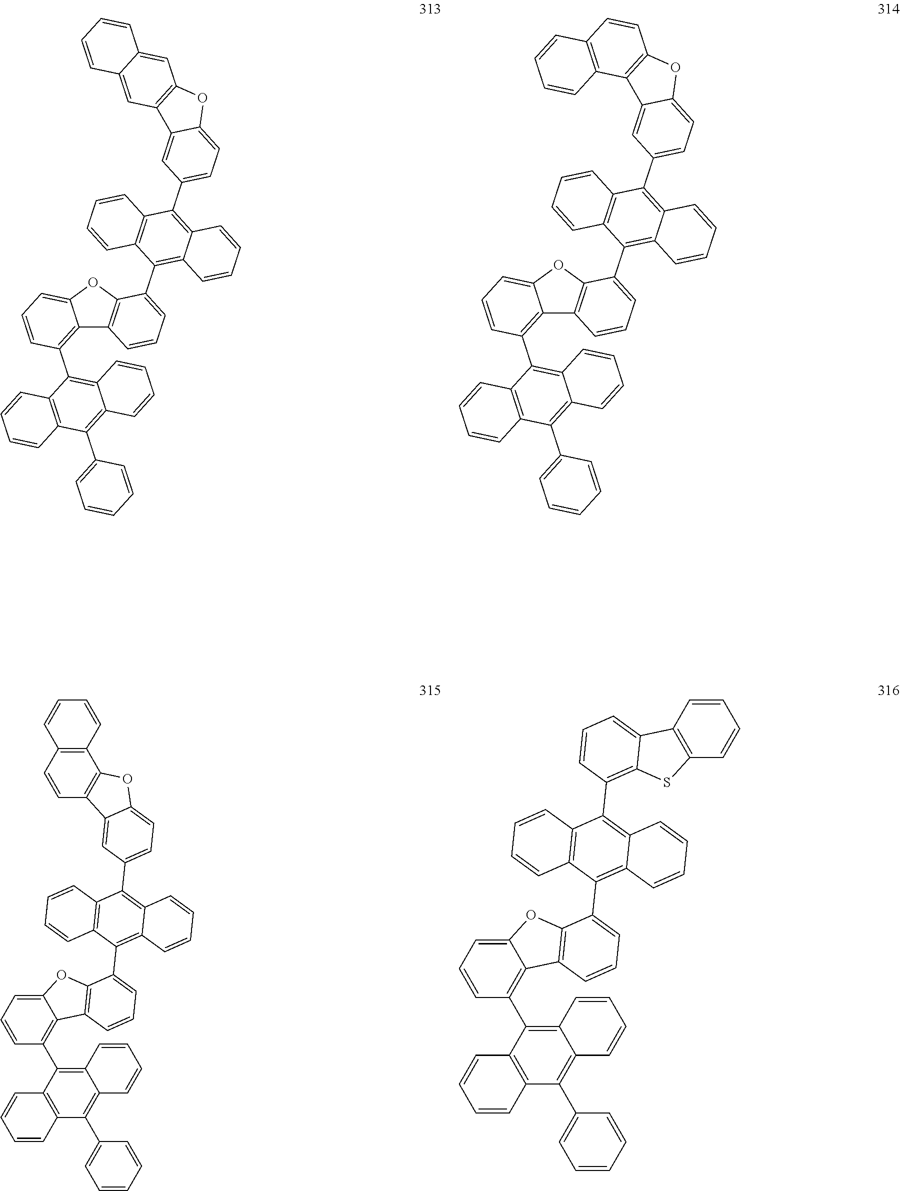 [SMILES: c1ccc(-c2c3ccccc3c(-c3cccc4oc5c(-c6c7ccccc7c(-c7ccc8oc9cc%10ccccc%10cc9c8c7)c7ccccc67)cccc5c34)c3ccccc23)cc1 ; c1ccc(-c2c3ccccc3c(-c3cccc4oc5c(-c6c7ccccc7c(-c7ccc8oc9ccc%10ccccc%10c9c8c7)c7ccccc67)cccc5c34)c3ccccc23)cc1 ; c1ccc(-c2c3ccccc3c(-c3cccc4oc5c(-c6c7ccccc7c(-c7ccc8oc9c%10ccccc%10ccc9c8c7)c7ccccc67)cccc5c34)c3ccccc23)cc1 ; c1ccc(-c2c3ccccc3c(-c3cccc4oc5c(-c6c7ccccc7c(-c7cccc8c7sc7ccccc78)c7ccccc67)cccc5c34)c3ccccc23)cc1]

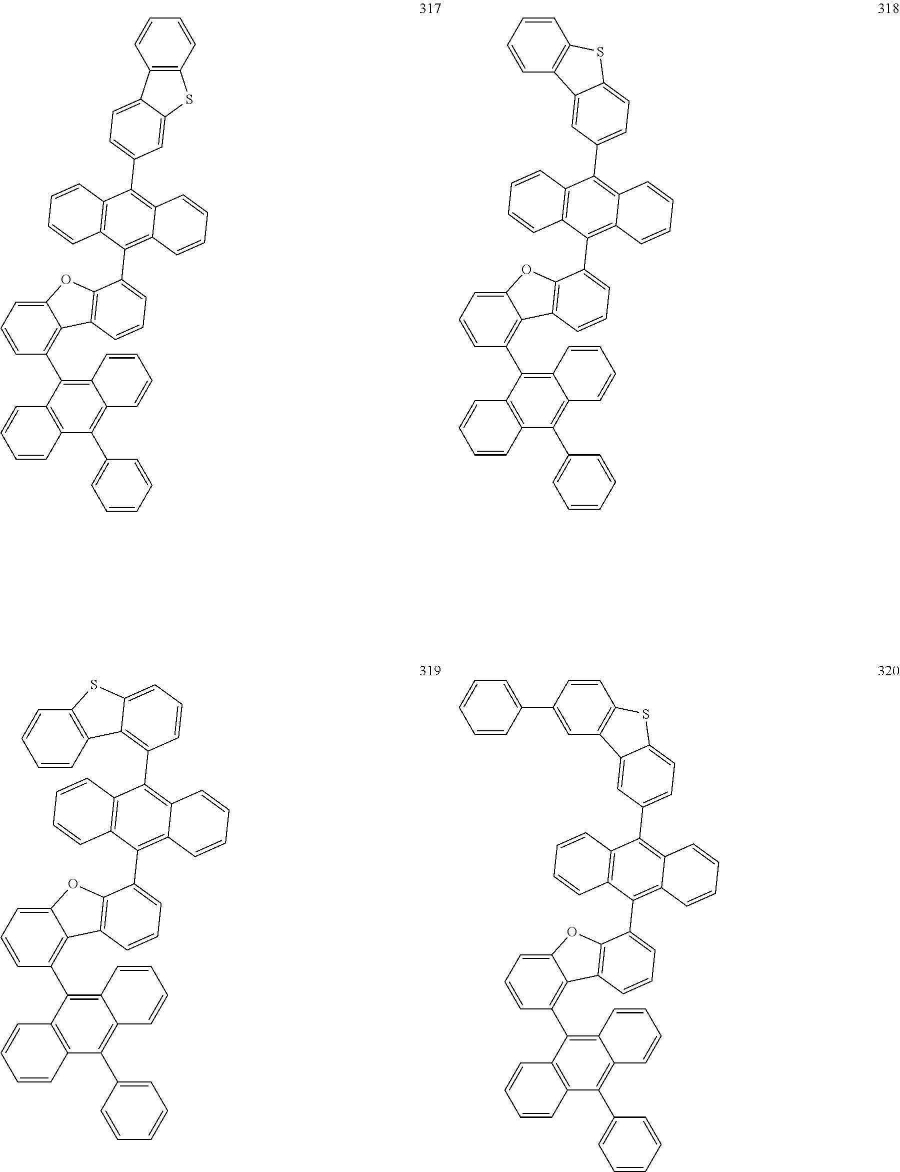 [SMILES: c1ccc(-c2c3ccccc3c(-c3cccc4oc5c(-c6c7ccccc7c(-c7ccc8c(c7)sc7ccccc78)c7ccccc67)cccc5c34)c3ccccc23)cc1 ; c1ccc(-c2c3ccccc3c(-c3cccc4oc5c(-c6c7ccccc7c(-c7ccc8sc9ccccc9c8c7)c7ccccc67)cccc5c34)c3ccccc23)cc1 ; c1ccc(-c2c3ccccc3c(-c3cccc4oc5c(-c6c7ccccc7c(-c7cccc8sc9ccccc9c78)c7ccccc67)cccc5c34)c3ccccc23)cc1 ; c1ccc(-c2ccc3sc4ccc(-c5c6ccccc6c(-c6cccc7c6oc6cccc(-c8c9ccccc9c(-c9ccccc9)c9ccccc89)c67)c6ccccc56)cc4c3c2)cc1]

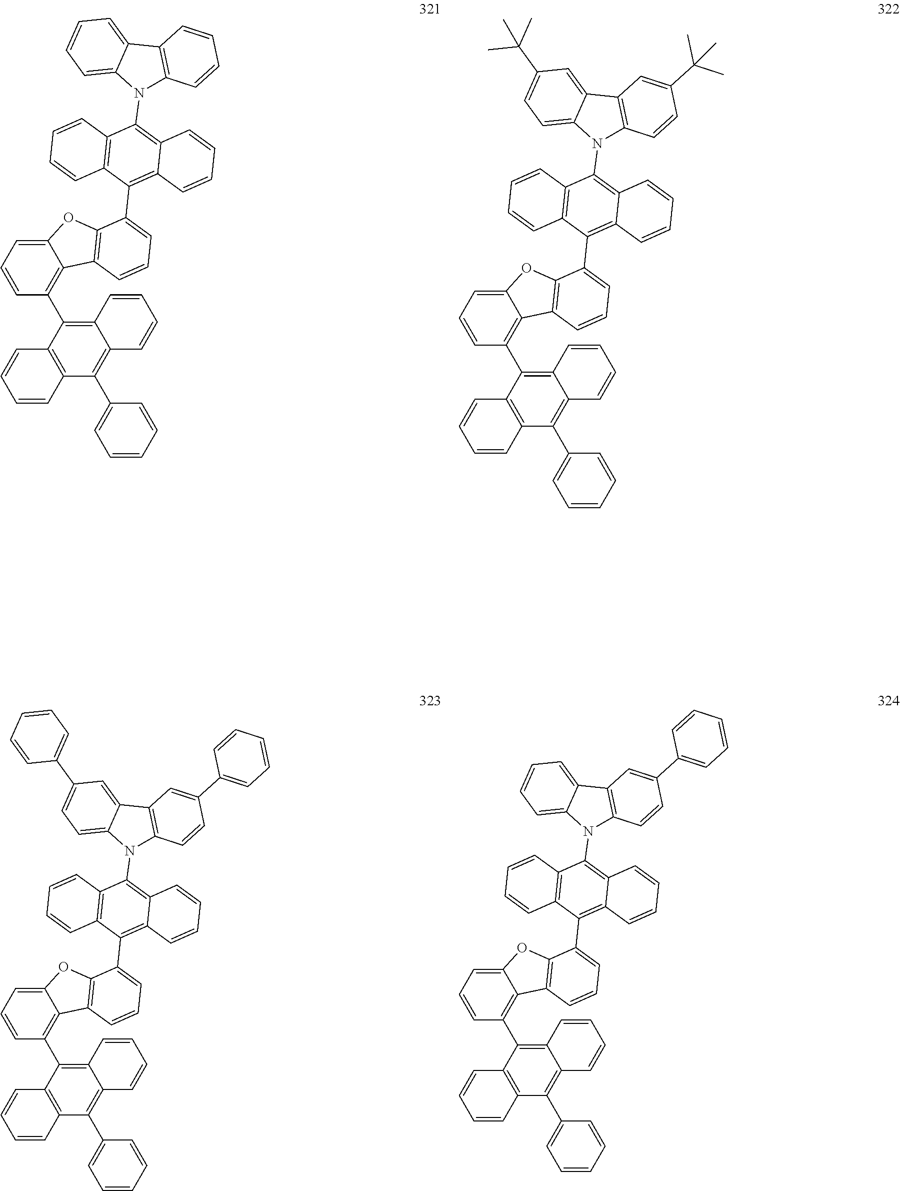 [SMILES: c1ccc(-c2c3ccccc3c(-c3cccc4oc5c(-c6c7ccccc7c(-n7c8ccccc8c8ccccc87)c7ccccc67)cccc5c34)c3ccccc23)cc1 ; CC(C)(C)c1ccc2c(c1)c1cc(C(C)(C)C)ccc1n2-c1c2ccccc2c(-c2cccc3c2oc2cccc(-c4c5ccccc5c(-c5ccccc5)c5ccccc45)c23)c2ccccc12 ; c1ccc(-c2ccc3c(c2)c2cc(-c4ccccc4)ccc2n3-c2c3ccccc3c(-c3cccc4c3oc3cccc(-c5c6ccccc6c(-c6ccccc6)c6ccccc56)c34)c3ccccc23)cc1 ; c1ccc(-c2ccc3c(c2)c2ccccc2n3-c2c3ccccc3c(-c3cccc4c3oc3cccc(-c5c6ccccc6c(-c6ccccc6)c6ccccc56)c34)c3ccccc23)cc1]

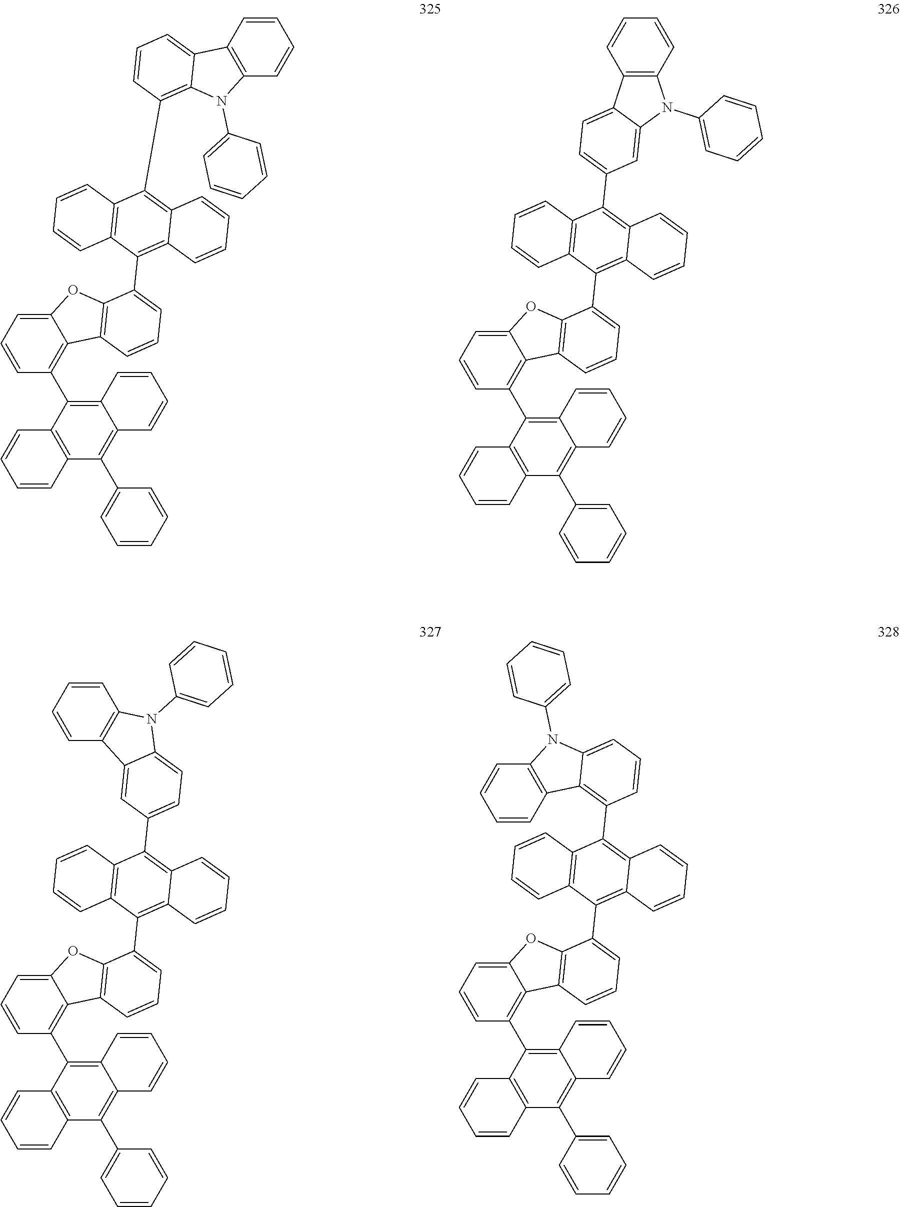 [SMILES: c1ccc(-c2c3ccccc3c(-c3cccc4oc5c(-c6c7ccccc7c(-c7cccc8c9ccccc9n(-c9ccccc9)c78)c7ccccc67)cccc5c34)c3ccccc23)cc1 ; c1ccc(-c2c3ccccc3c(-c3cccc4oc5c(-c6c7ccccc7c(-c7ccc8c9ccccc9n(-c9ccccc9)c8c7)c7ccccc67)cccc5c34)c3ccccc23)cc1 ; c1ccc(-c2c3ccccc3c(-c3cccc4oc5c(-c6c7ccccc7c(-c7ccc8c(c7)c7ccccc7n8-c7ccccc7)c7ccccc67)cccc5c34)c3ccccc23)cc1 ; c1ccc(-c2c3ccccc3c(-c3cccc4oc5c(-c6c7ccccc7c(-c7cccc8c7c7ccccc7n8-c7ccccc7)c7ccccc67)cccc5c34)c3ccccc23)cc1]

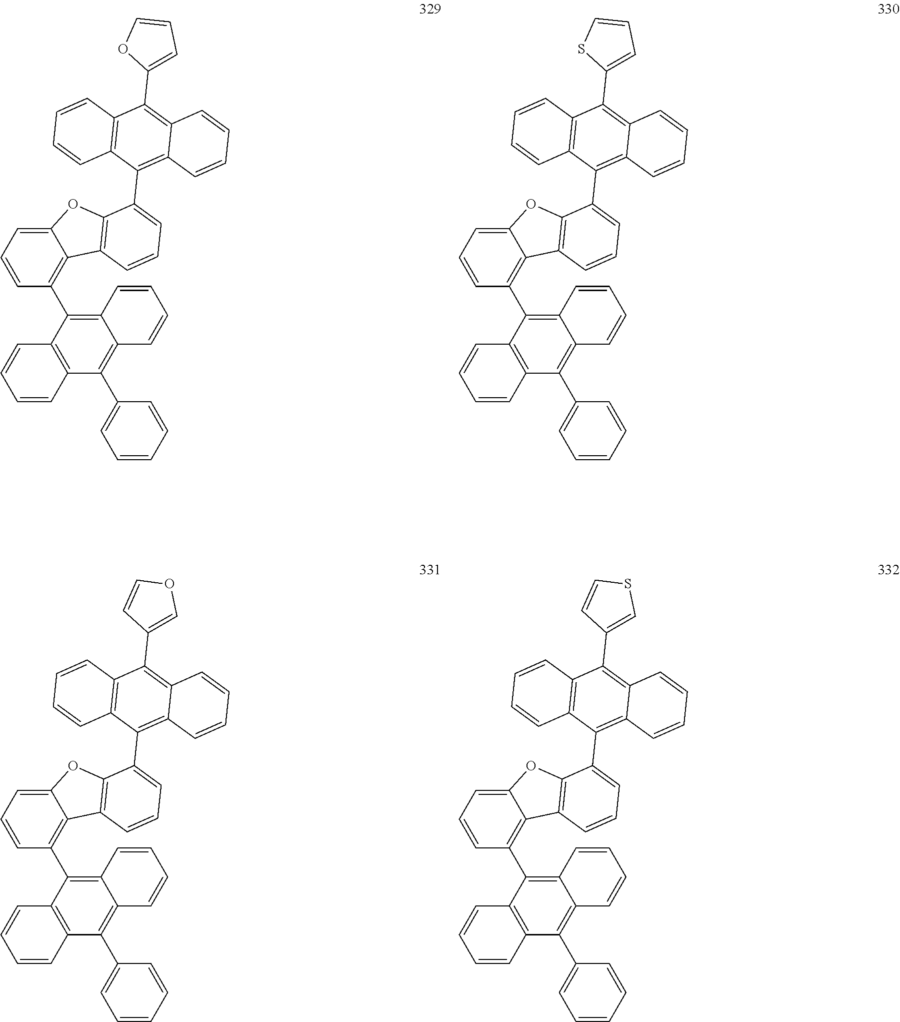 [SMILES: c1ccc(-c2c3ccccc3c(-c3cccc4oc5c(-c6c7ccccc7c(-c7ccco7)c7ccccc67)cccc5c34)c3ccccc23)cc1 ; c1ccc(-c2c3ccccc3c(-c3cccc4oc5c(-c6c7ccccc7c(-c7cccs7)c7ccccc67)cccc5c34)c3ccccc23)cc1 ; c1ccc(-c2c3ccccc3c(-c3cccc4oc5c(-c6c7ccccc7c(-c7ccoc7)c7ccccc67)cccc5c34)c3ccccc23)cc1 ; c1ccc(-c2c3ccccc3c(-c3cccc4oc5c(-c6c7ccccc7c(-c7ccsc7)c7ccccc67)cccc5c34)c3ccccc23)cc1]

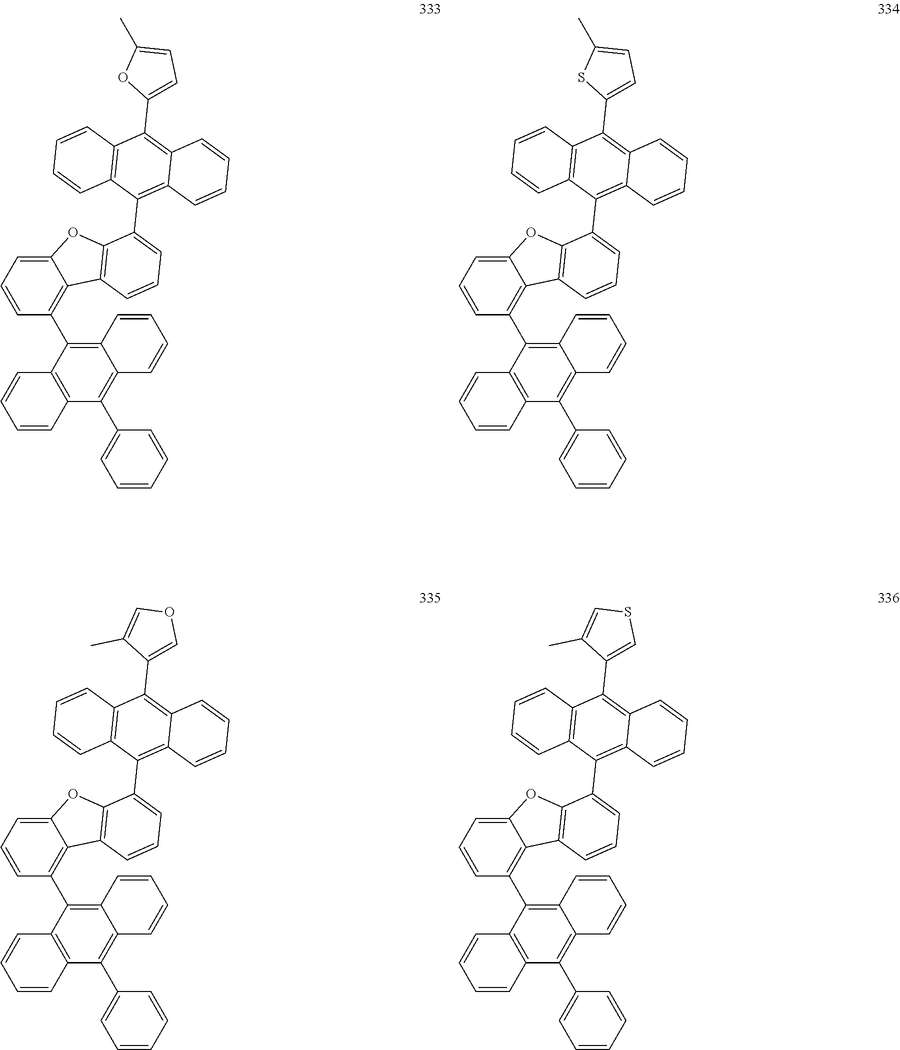 [SMILES: Cc1ccc(-c2c3ccccc3c(-c3cccc4c3oc3cccc(-c5c6ccccc6c(-c6ccccc6)c6ccccc56)c34)c3ccccc23)o1 ; Cc1ccc(-c2c3ccccc3c(-c3cccc4c3oc3cccc(-c5c6ccccc6c(-c6ccccc6)c6ccccc56)c34)c3ccccc23)s1 ; Cc1cocc1-c1c2ccccc2c(-c2cccc3c2oc2cccc(-c4c5ccccc5c(-c5ccccc5)c5ccccc45)c23)c2ccccc12 ; Cc1cscc1-c1c2ccccc2c(-c2cccc3c2oc2cccc(-c4c5ccccc5c(-c5ccccc5)c5ccccc45)c23)c2ccccc12]

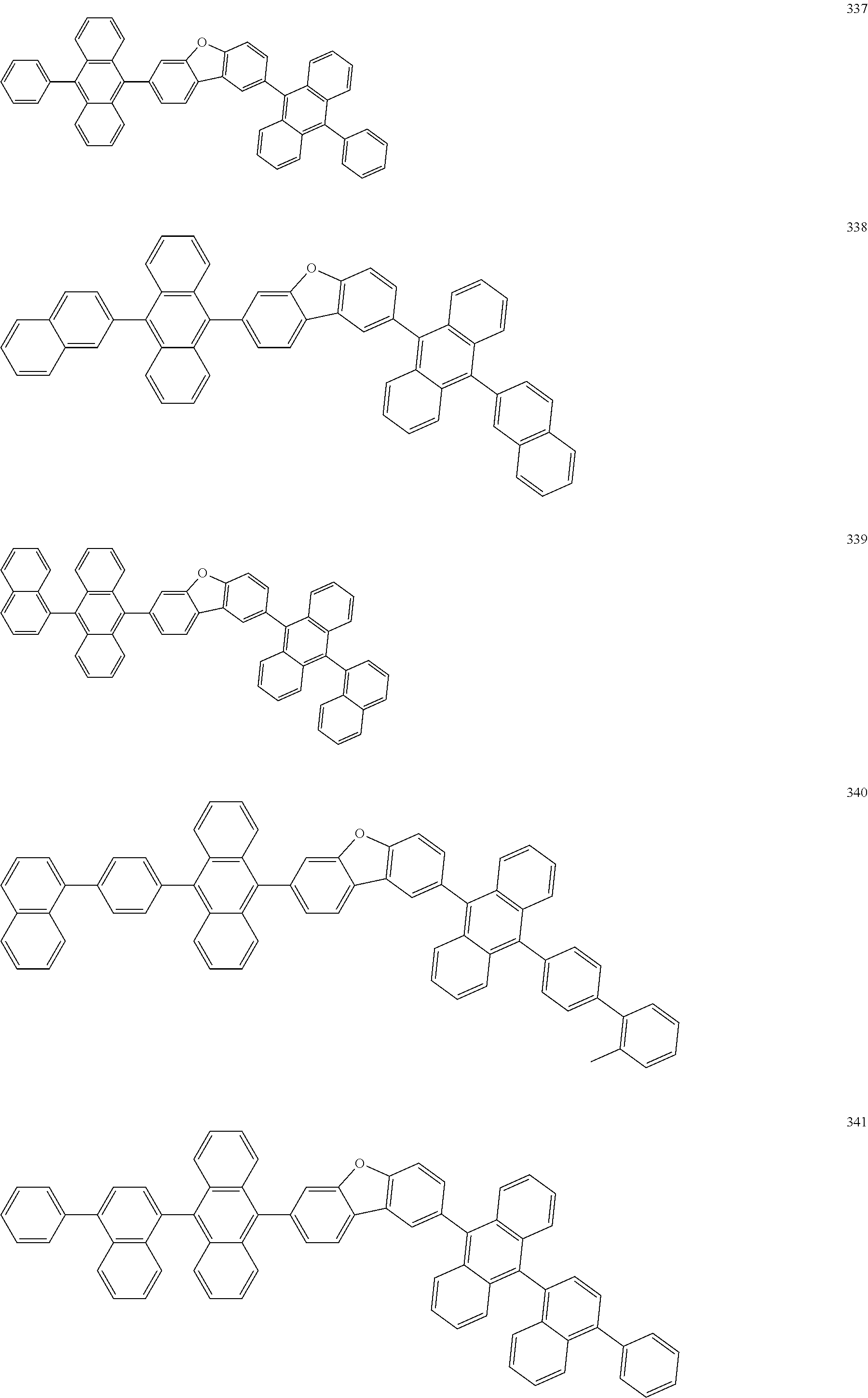

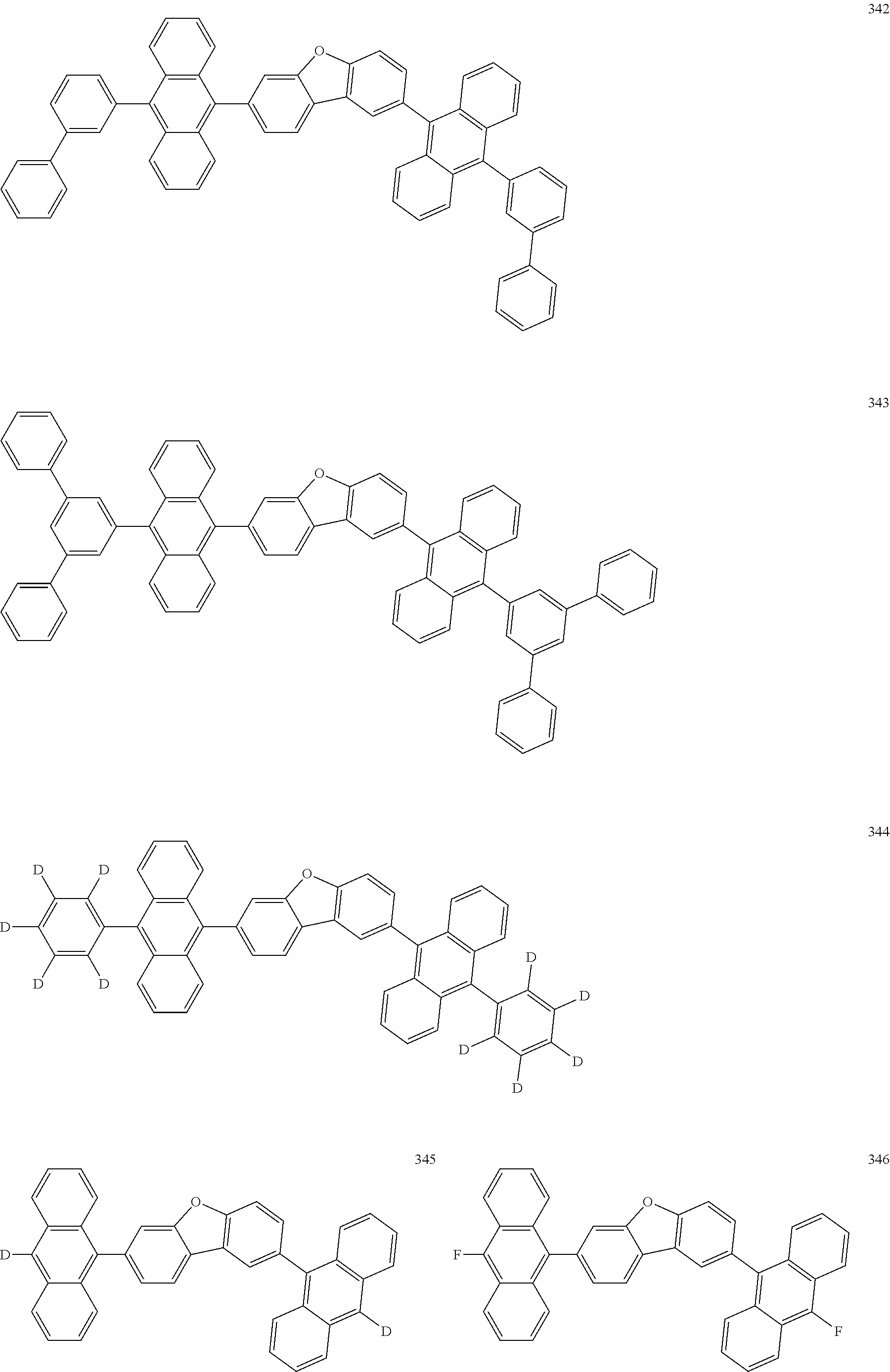

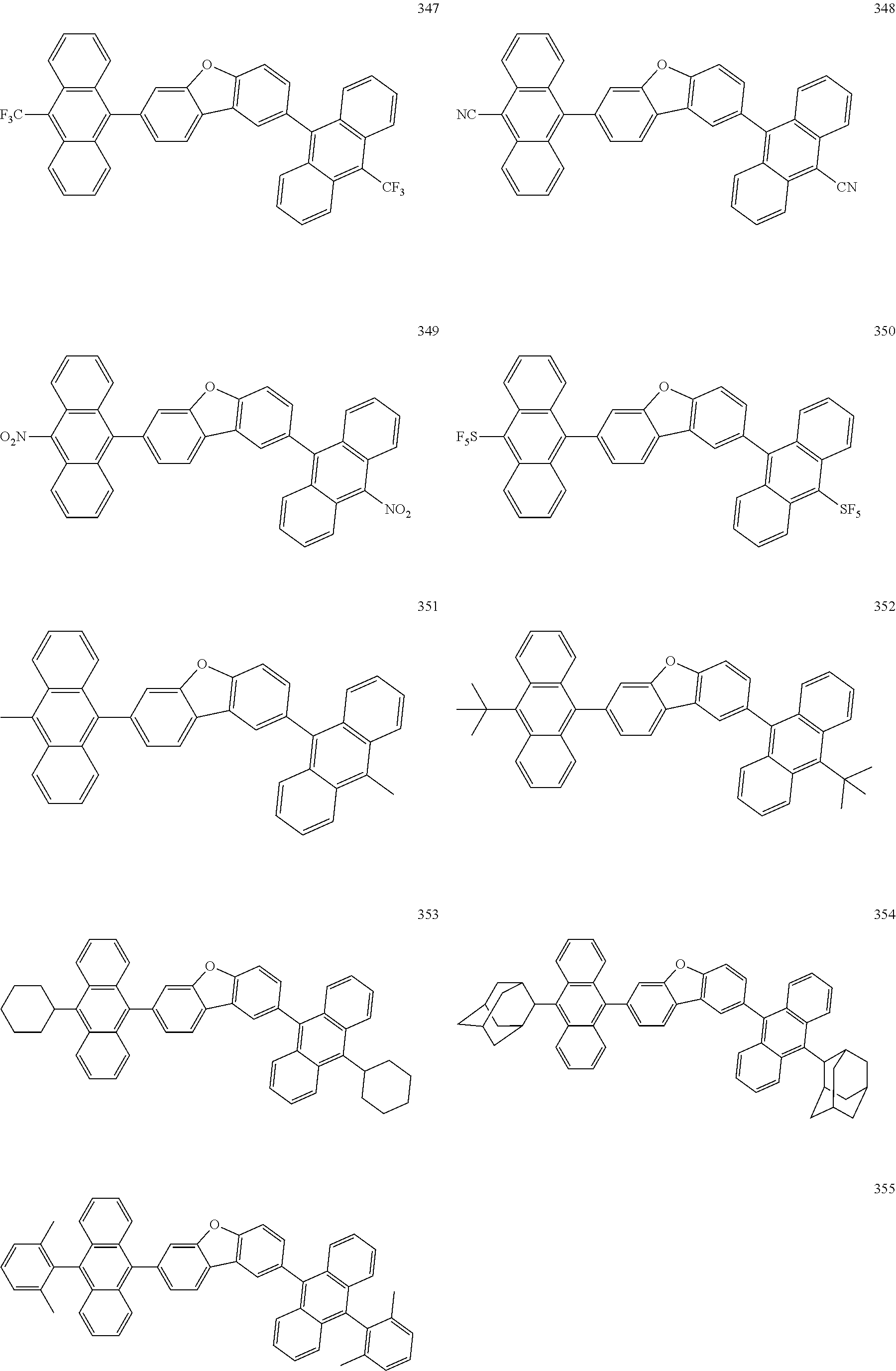

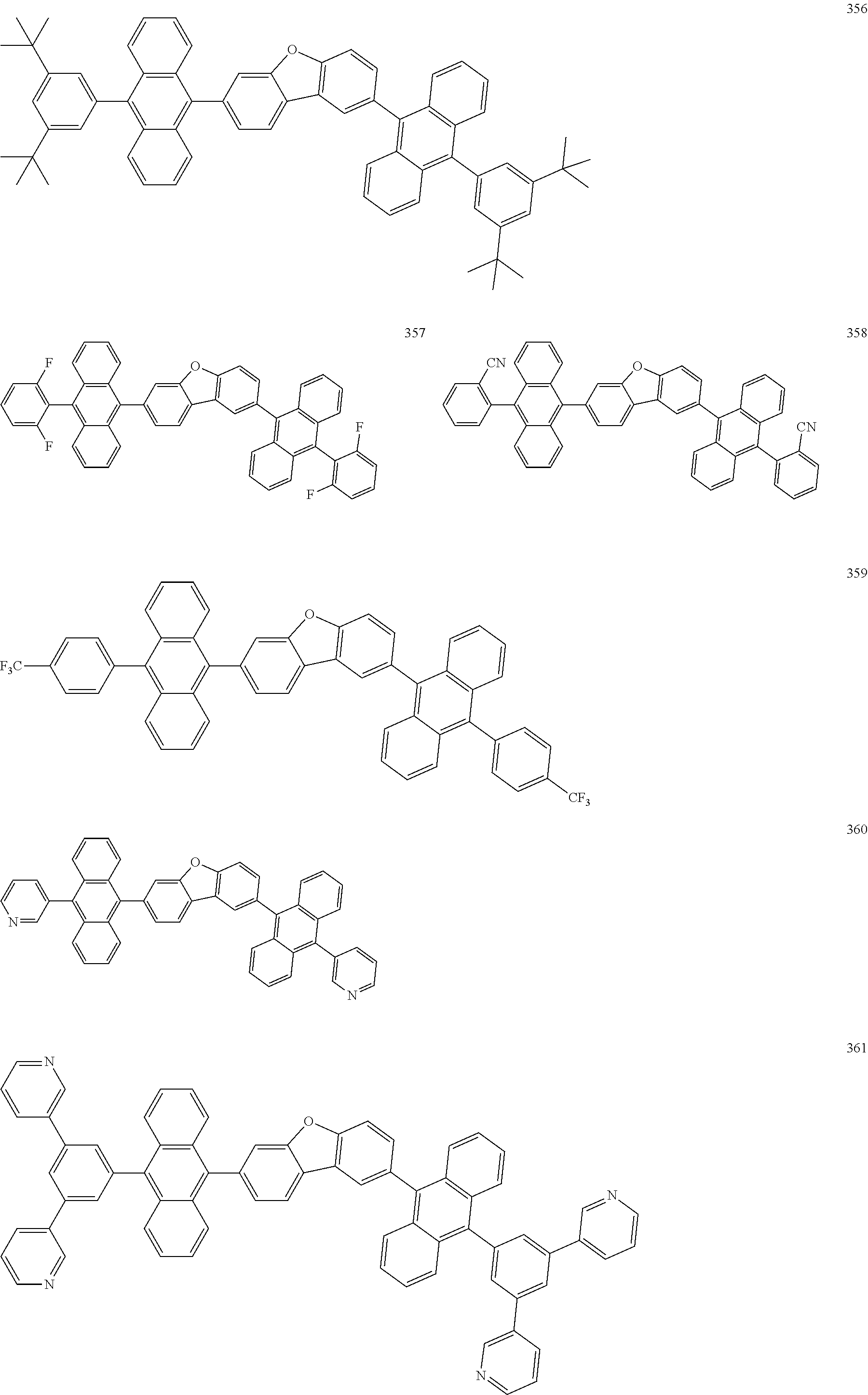

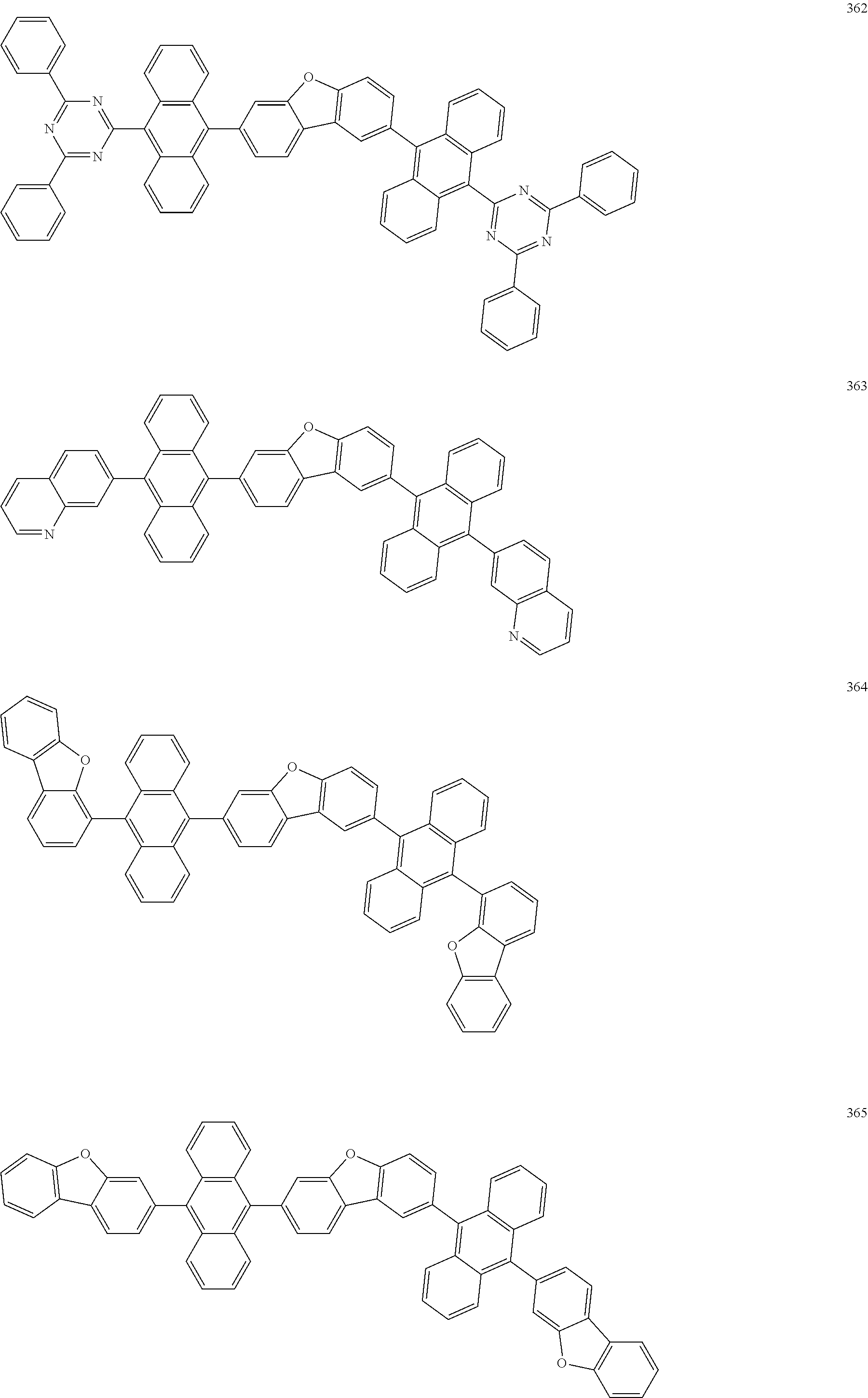 [SMILES: c1ccc(-c2nc(-c3ccccc3)nc(-c3c4ccccc4c(-c4ccc5c(c4)oc4ccc(-c6c7ccccc7c(-c7nc(-c8ccccc8)nc(-c8ccccc8)n7)c7ccccc67)cc45)c4ccccc34)n2)cc1 ; c1cnc2cc(-c3c4ccccc4c(-c4ccc5c(c4)oc4ccc(-c6c7ccccc7c(-c7ccc8cccnc8c7)c7ccccc67)cc45)c4ccccc34)ccc2c1 ; c1ccc2c(c1)oc1c(-c3c4ccccc4c(-c4ccc5c(c4)oc4ccc(-c6c7ccccc7c(-c7cccc8c7oc7ccccc78)c7ccccc67)cc45)c4ccccc34)cccc12 ; c1ccc2c(c1)oc1cc(-c3c4ccccc4c(-c4ccc5c(c4)oc4ccc(-c6c7ccccc7c(-c7ccc8c(c7)oc7ccccc78)c7ccccc67)cc45)c4ccccc34)ccc12]

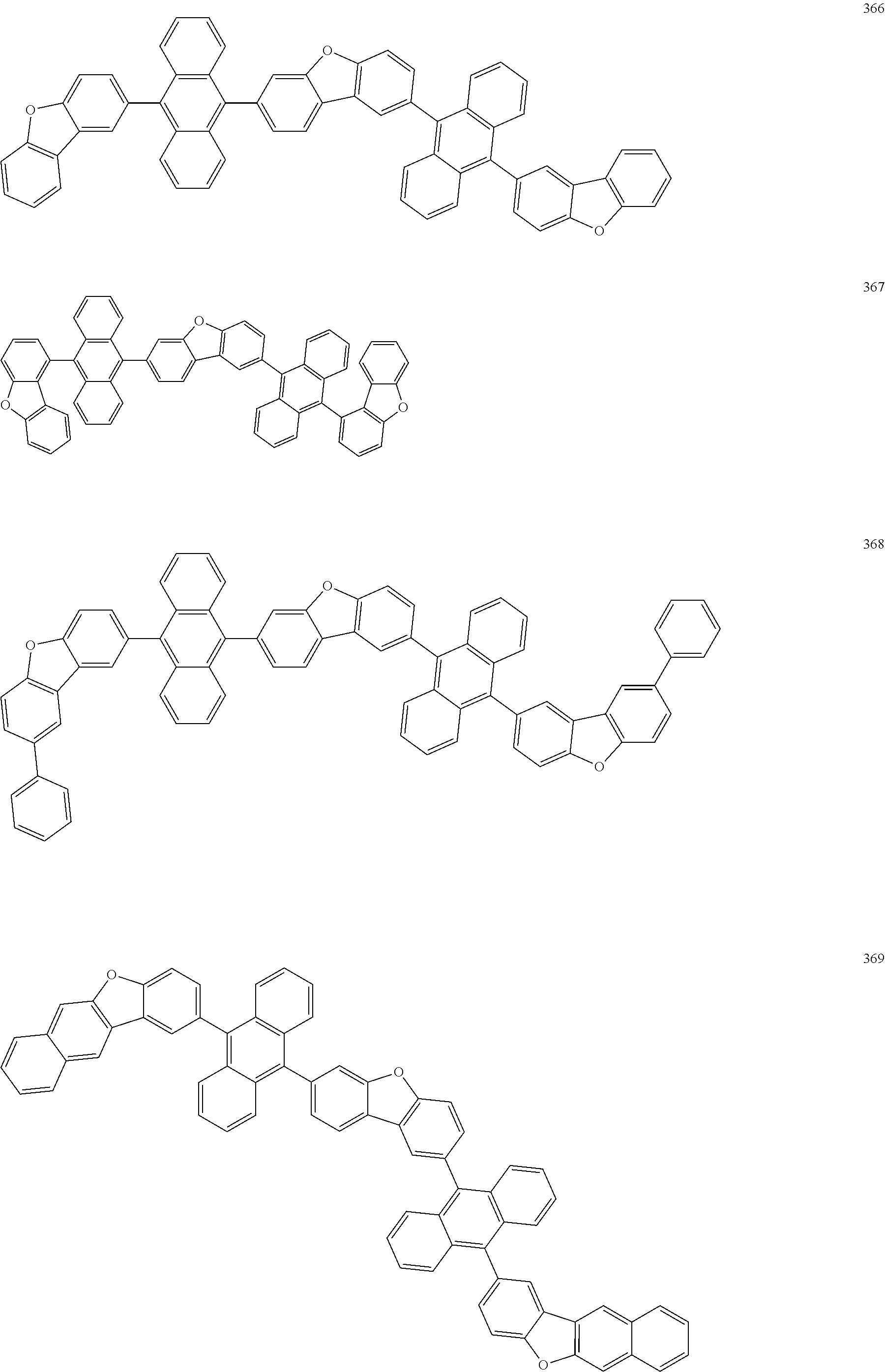

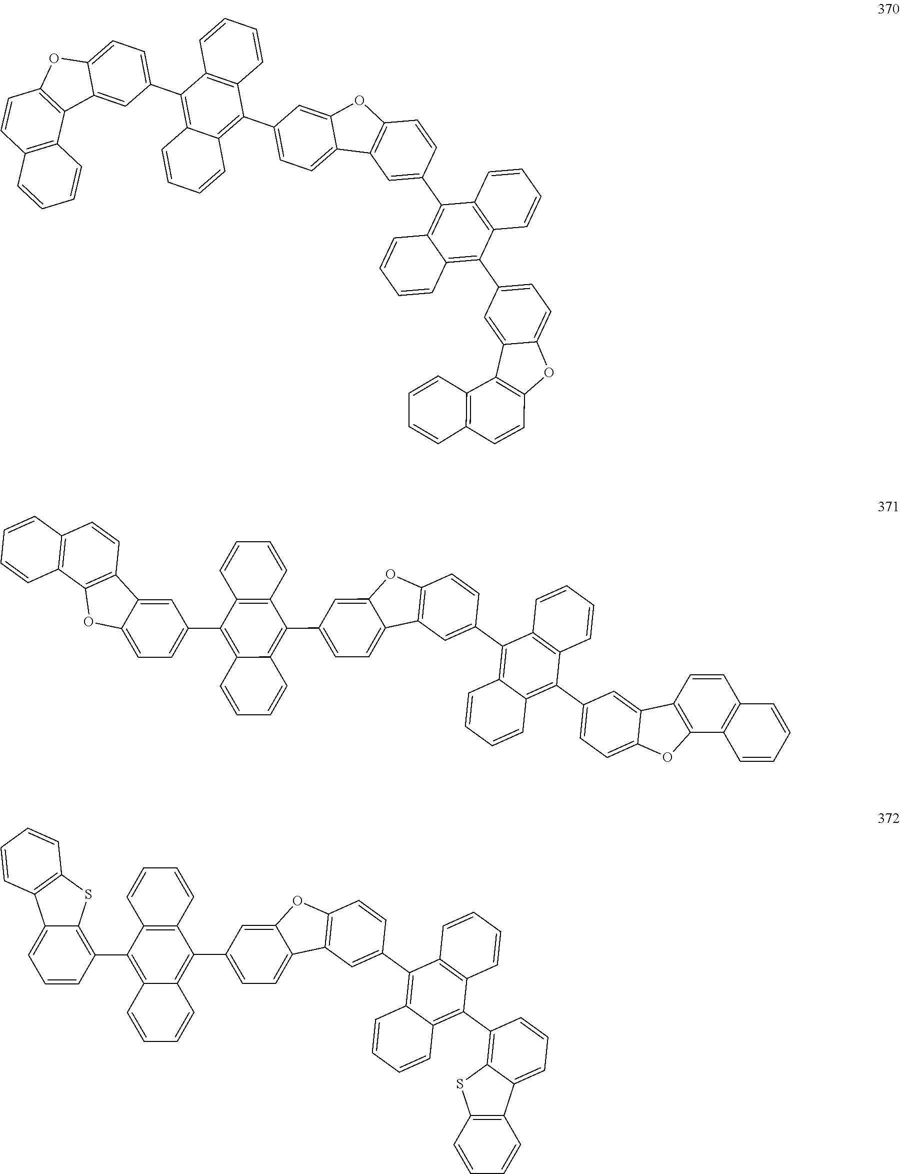 [SMILES: c1ccc2c(c1)ccc1oc3ccc(-c4c5ccccc5c(-c5ccc6c(c5)oc5ccc(-c7c8ccccc8c(-c8ccc9oc%10ccc%11ccccc%11c%10c9c8)c8ccccc78)cc56)c5ccccc45)cc3c12 ; c1ccc2c(c1)ccc1c3cc(-c4c5ccccc5c(-c5ccc6c(c5)oc5ccc(-c7c8ccccc8c(-c8ccc9oc%10c%11ccccc%11ccc%10c9c8)c8ccccc78)cc56)c5ccccc45)ccc3oc21 ; c1ccc2c(c1)sc1c(-c3c4ccccc4c(-c4ccc5c(c4)oc4ccc(-c6c7ccccc7c(-c7cccc8c7sc7ccccc78)c7ccccc67)cc45)c4ccccc34)cccc12]

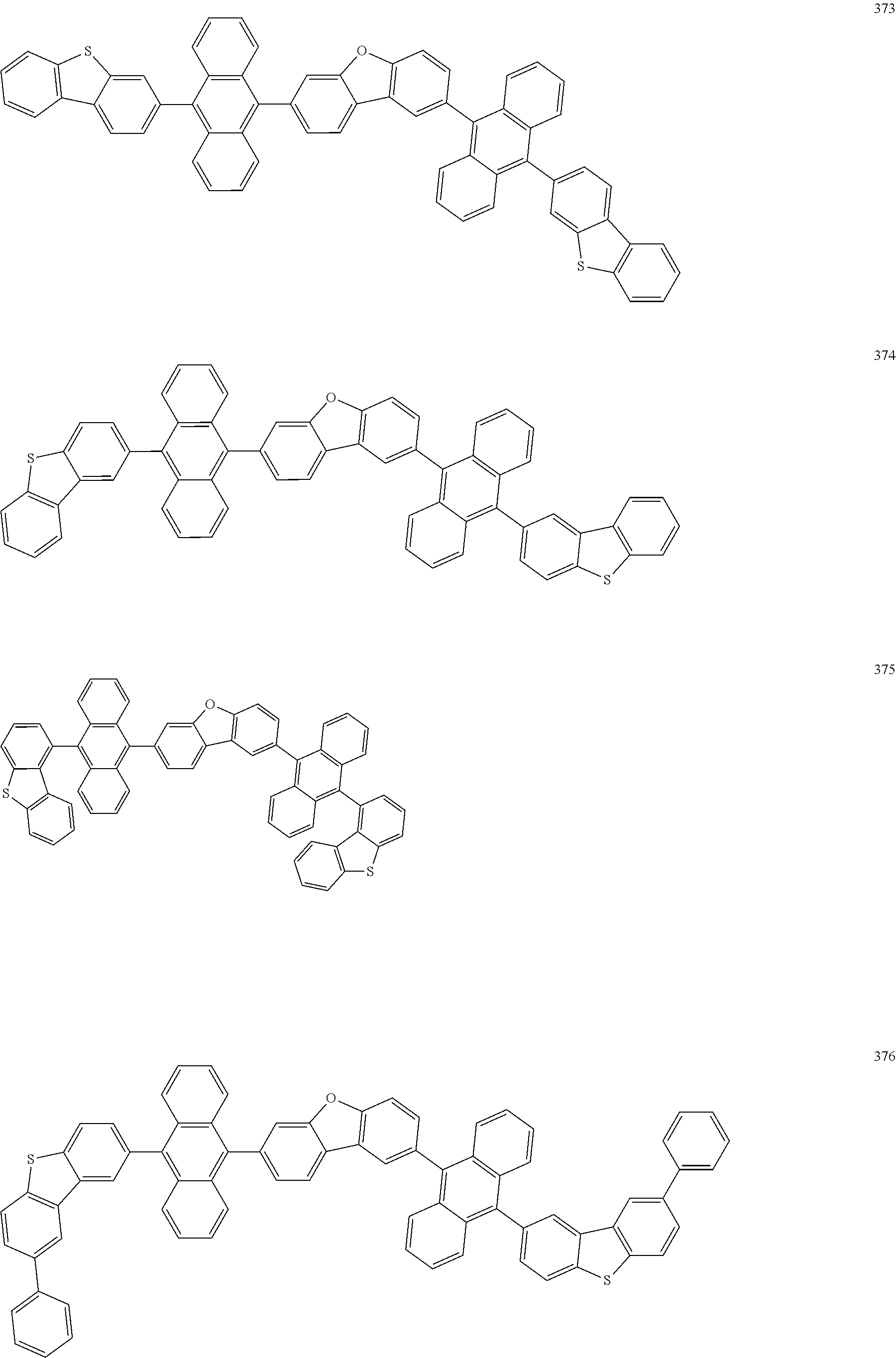 [SMILES: c1ccc2c(c1)sc1cc(-c3c4ccccc4c(-c4ccc5c(c4)oc4ccc(-c6c7ccccc7c(-c7ccc8c(c7)sc7ccccc78)c7ccccc67)cc45)c4ccccc34)ccc12 ; c1ccc2c(c1)sc1ccc(-c3c4ccccc4c(-c4ccc5c(c4)oc4ccc(-c6c7ccccc7c(-c7ccc8sc9ccccc9c8c7)c7ccccc67)cc45)c4ccccc34)cc12 ; c1ccc2c(c1)sc1cccc(-c3c4ccccc4c(-c4ccc5c(c4)oc4ccc(-c6c7ccccc7c(-c7cccc8sc9ccccc9c78)c7ccccc67)cc45)c4ccccc34)c12 ; c1ccc(-c2ccc3sc4ccc(-c5c6ccccc6c(-c6ccc7c(c6)oc6ccc(-c8c9ccccc9c(-c9ccc%10sc%11ccc(-c%12ccccc%12)cc%11c%10c9)c9ccccc89)cc67)c6ccccc56)cc4c3c2)cc1]

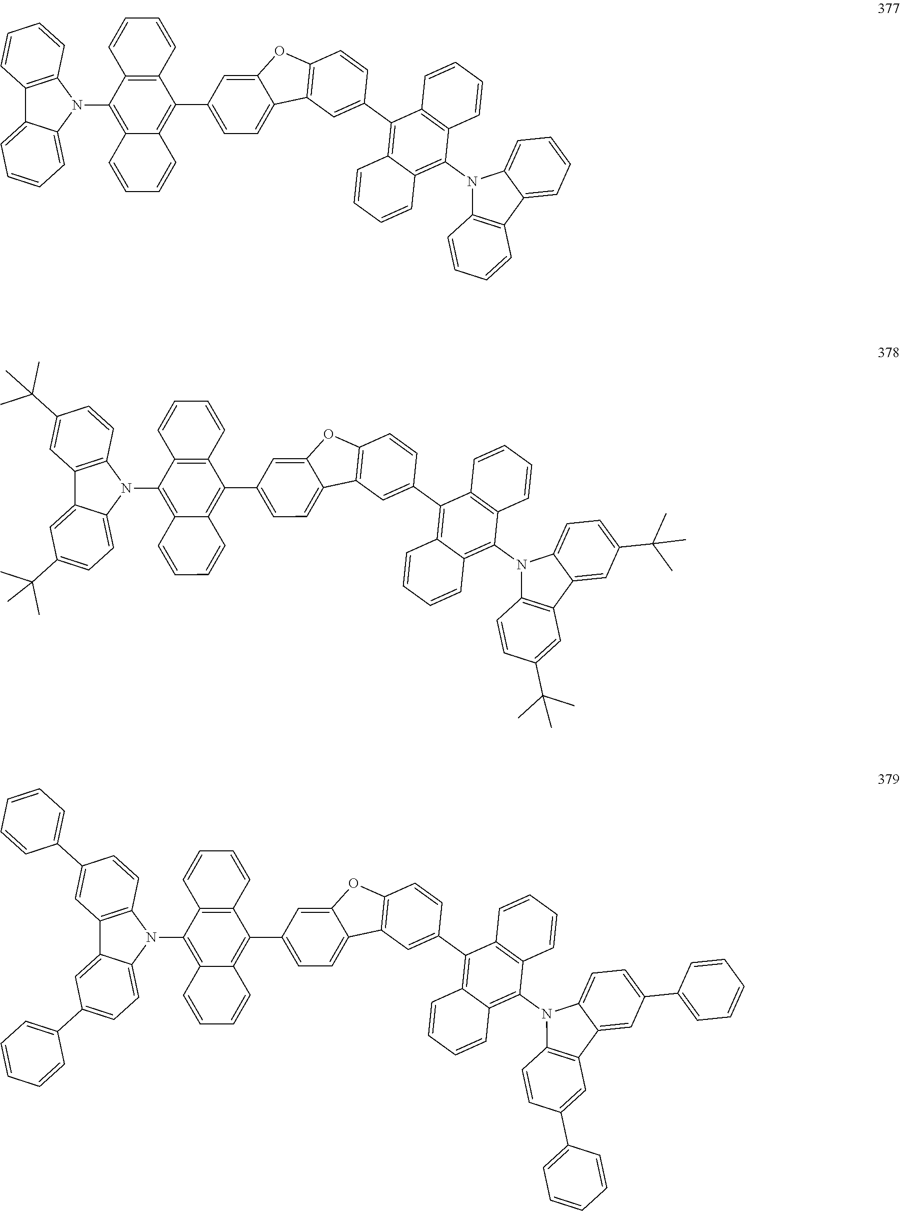 [SMILES: c1ccc2c(-n3c4ccccc4c4ccccc43)c3ccccc3c(-c3ccc4c(c3)oc3ccc(-c5c6ccccc6c(-n6c7ccccc7c7ccccc76)c6ccccc56)cc34)c2c1 ; CC(C)(C)c1ccc2c(c1)c1cc(C(C)(C)C)ccc1n2-c1c2ccccc2c(-c2ccc3c(c2)oc2ccc(-c4c5ccccc5c(-n5c6ccc(C(C)(C)C)cc6c6cc(C(C)(C)C)ccc65)c5ccccc45)cc23)c2ccccc12 ; c1ccc(-c2ccc3c(c2)c2cc(-c4ccccc4)ccc2n3-c2c3ccccc3c(-c3ccc4c(c3)oc3ccc(-c5c6ccccc6c(-n6c7ccc(-c8ccccc8)cc7c7cc(-c8ccccc8)ccc76)c6ccccc56)cc34)c3ccccc23)cc1]

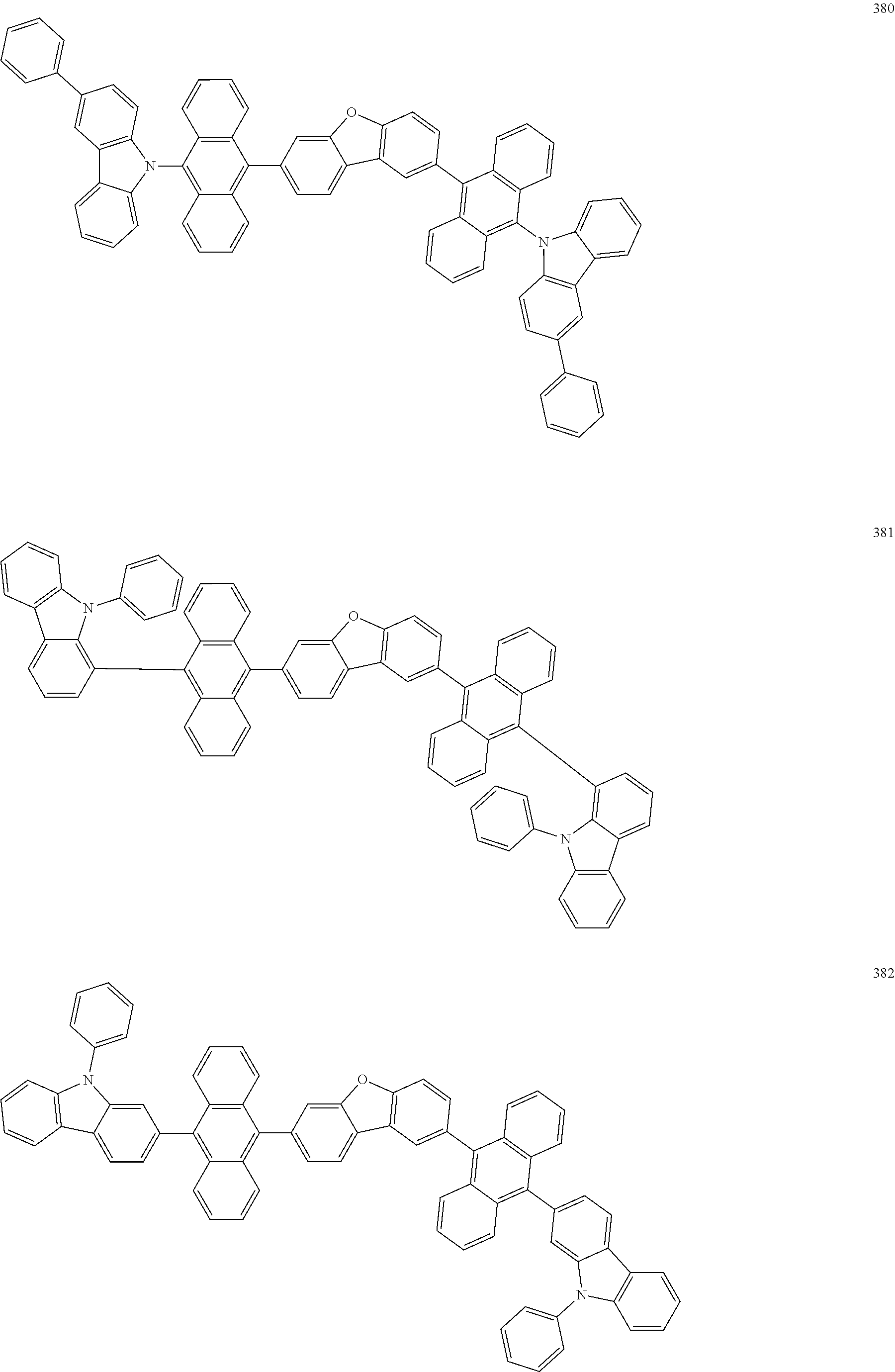 [SMILES: c1ccc(-c2ccc3c(c2)c2ccccc2n3-c2c3ccccc3c(-c3ccc4c(c3)oc3ccc(-c5c6ccccc6c(-n6c7ccccc7c7cc(-c8ccccc8)ccc76)c6ccccc56)cc34)c3ccccc23)cc1 ; c1ccc(-n2c3ccccc3c3cccc(-c4c5ccccc5c(-c5ccc6c(c5)oc5ccc(-c7c8ccccc8c(-c8cccc9c%10ccccc%10n(-c%10ccccc%10)c89)c8ccccc78)cc56)c5ccccc45)c32)cc1 ; c1ccc(-n2c3ccccc3c3ccc(-c4c5ccccc5c(-c5ccc6c(c5)oc5ccc(-c7c8ccccc8c(-c8ccc9c%10ccccc%10n(-c%10ccccc%10)c9c8)c8ccccc78)cc56)c5ccccc45)cc32)cc1]

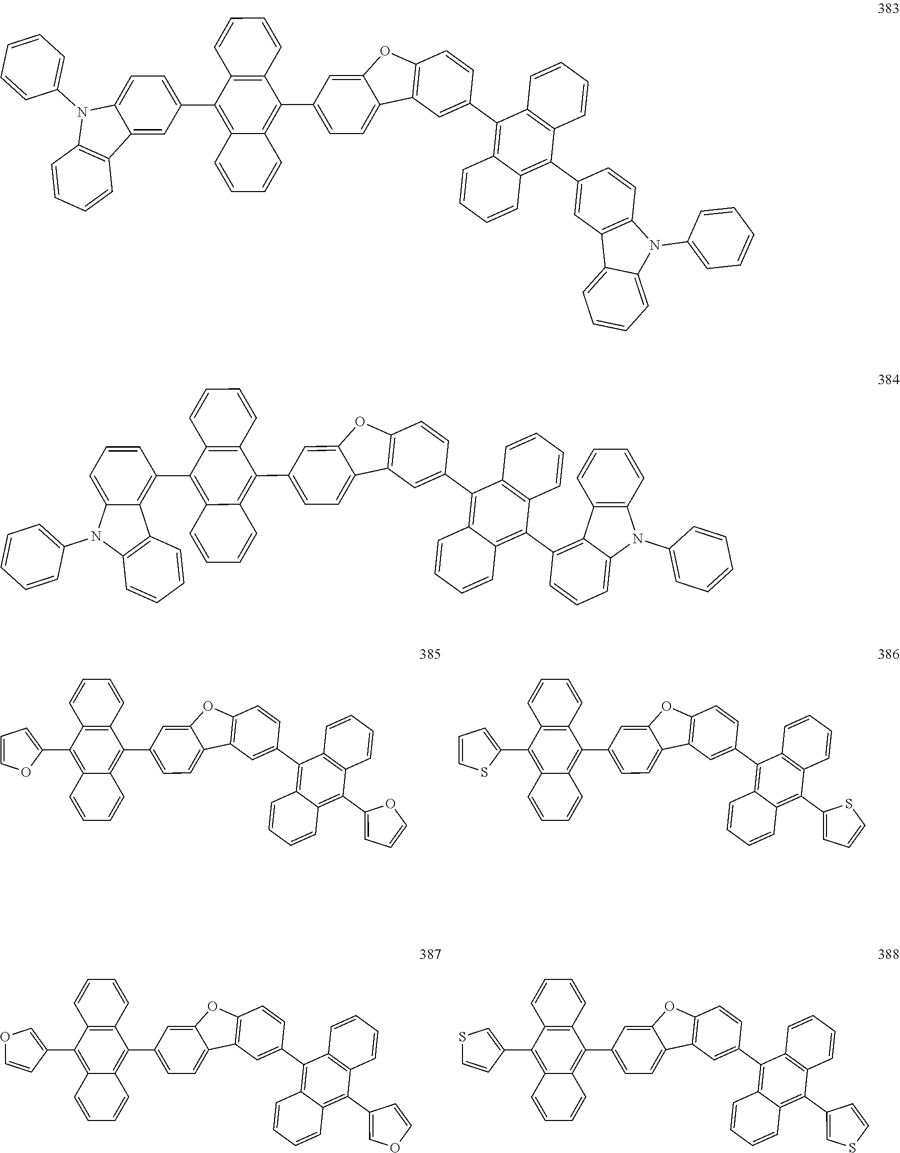

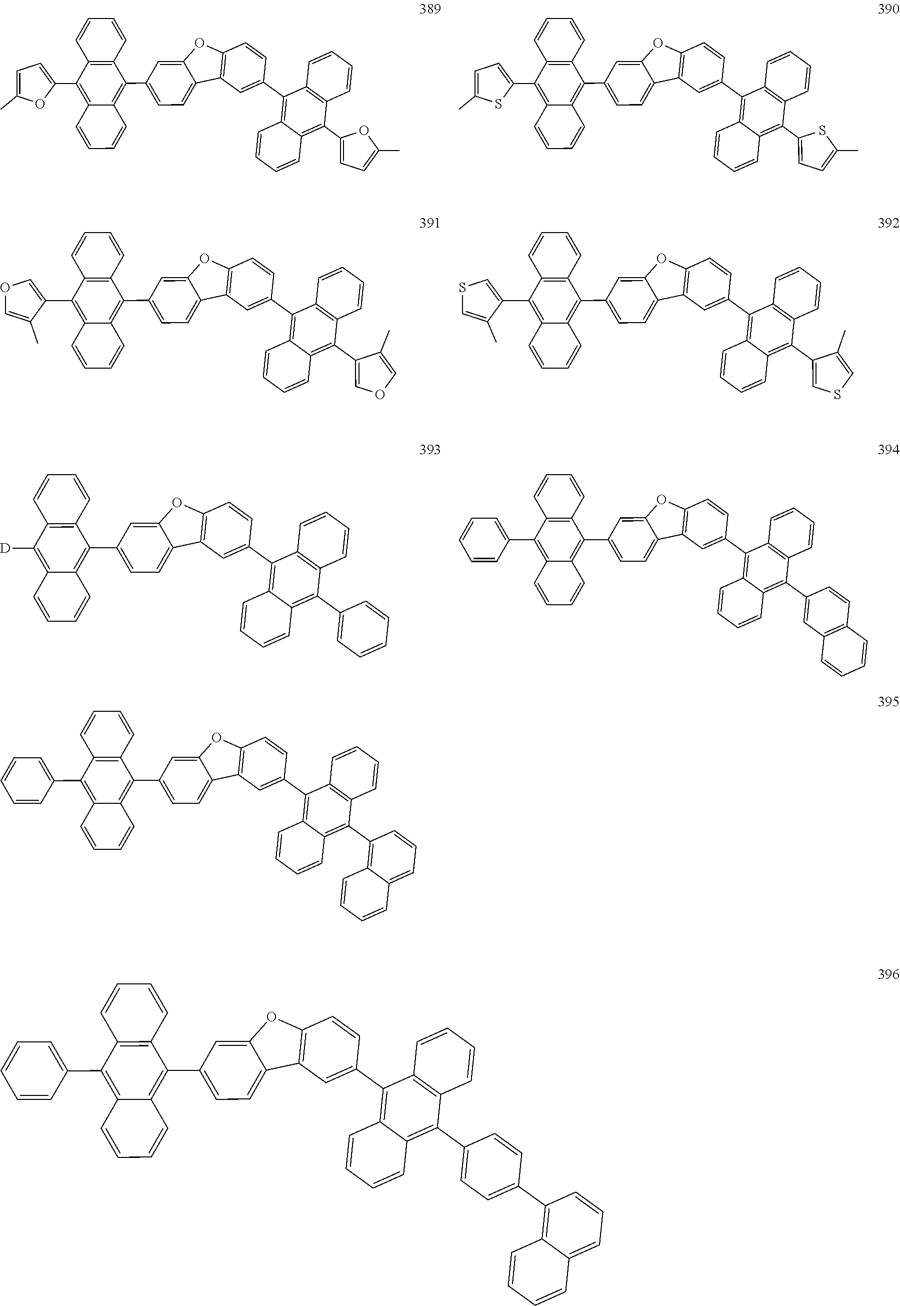

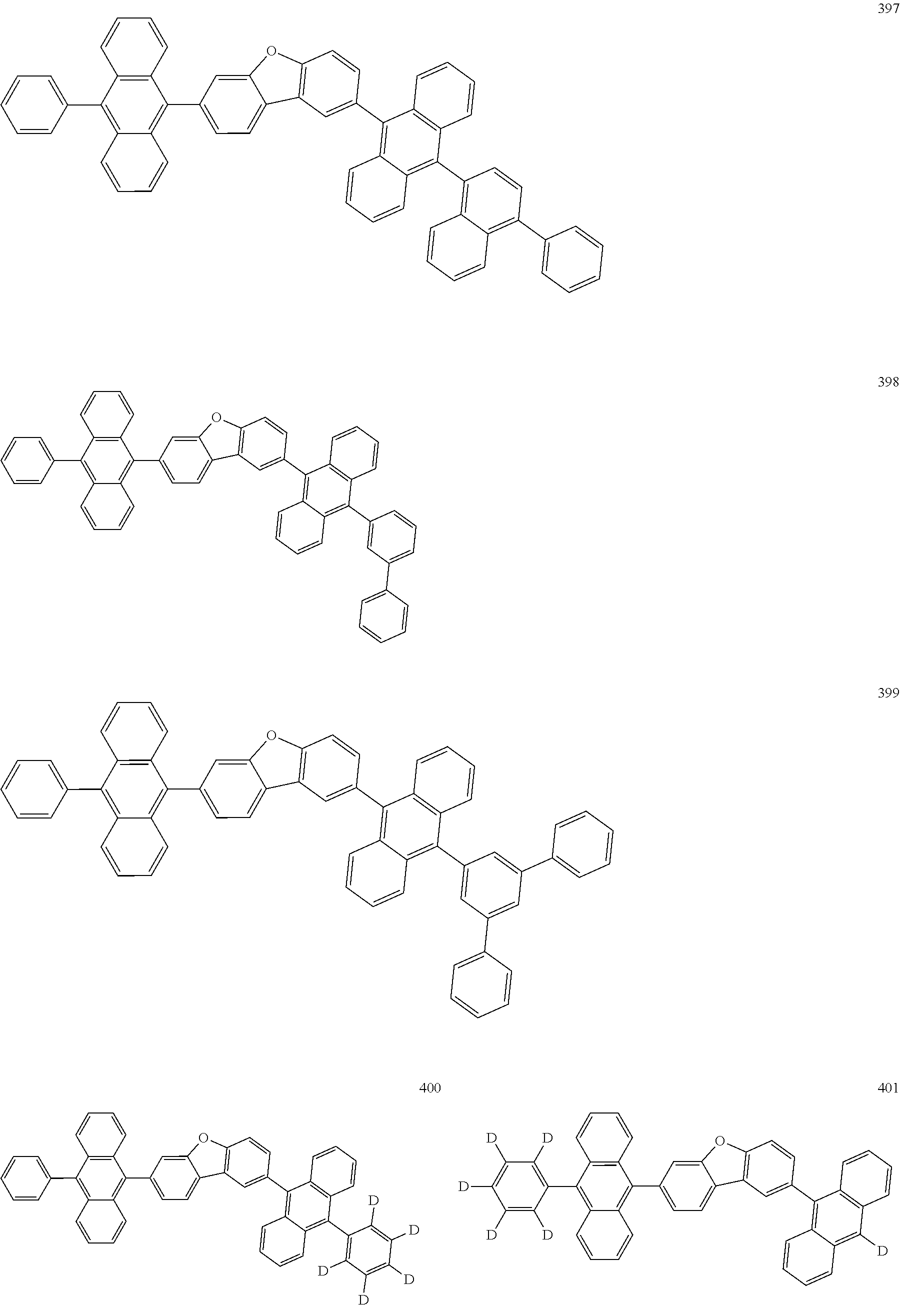

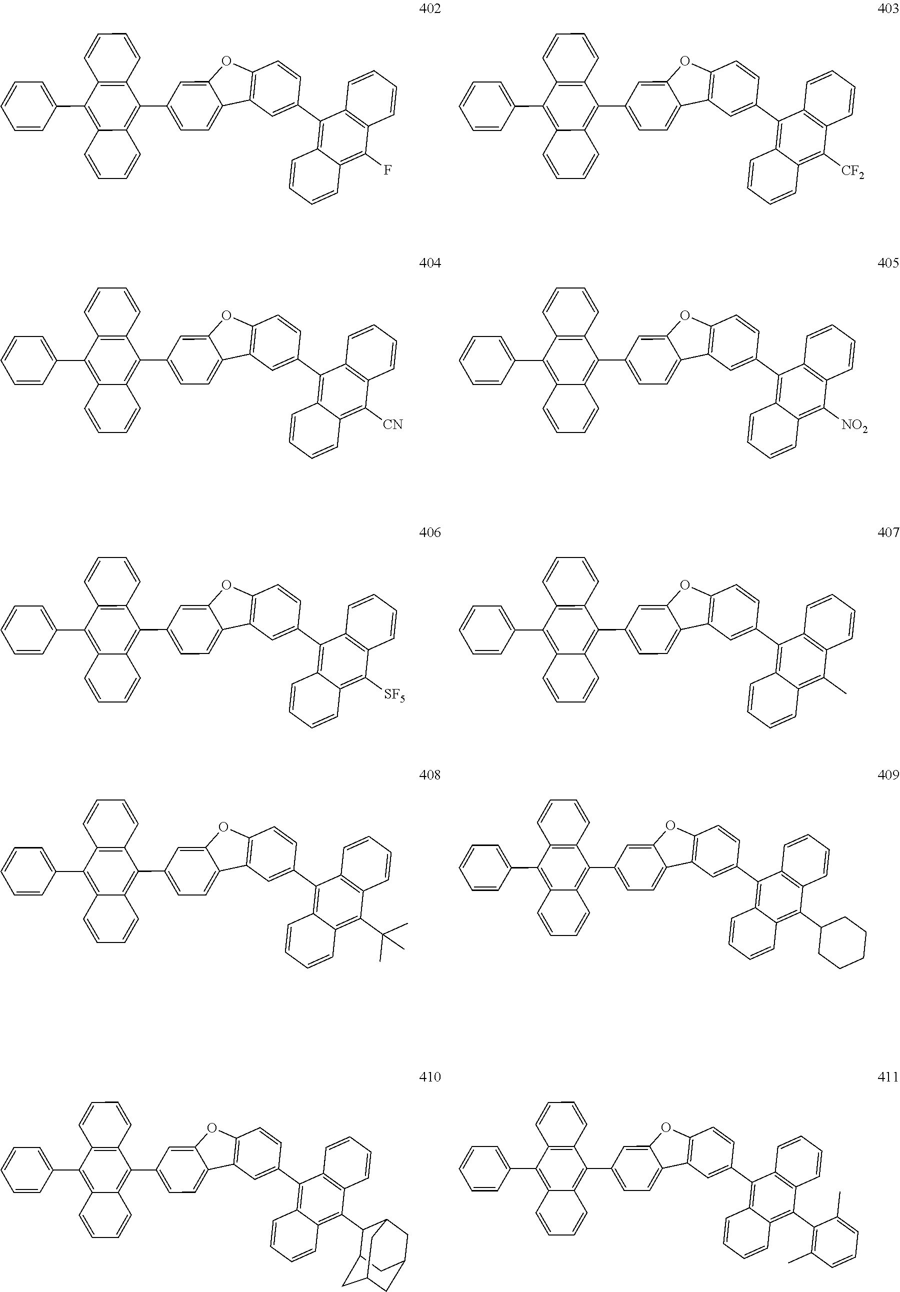

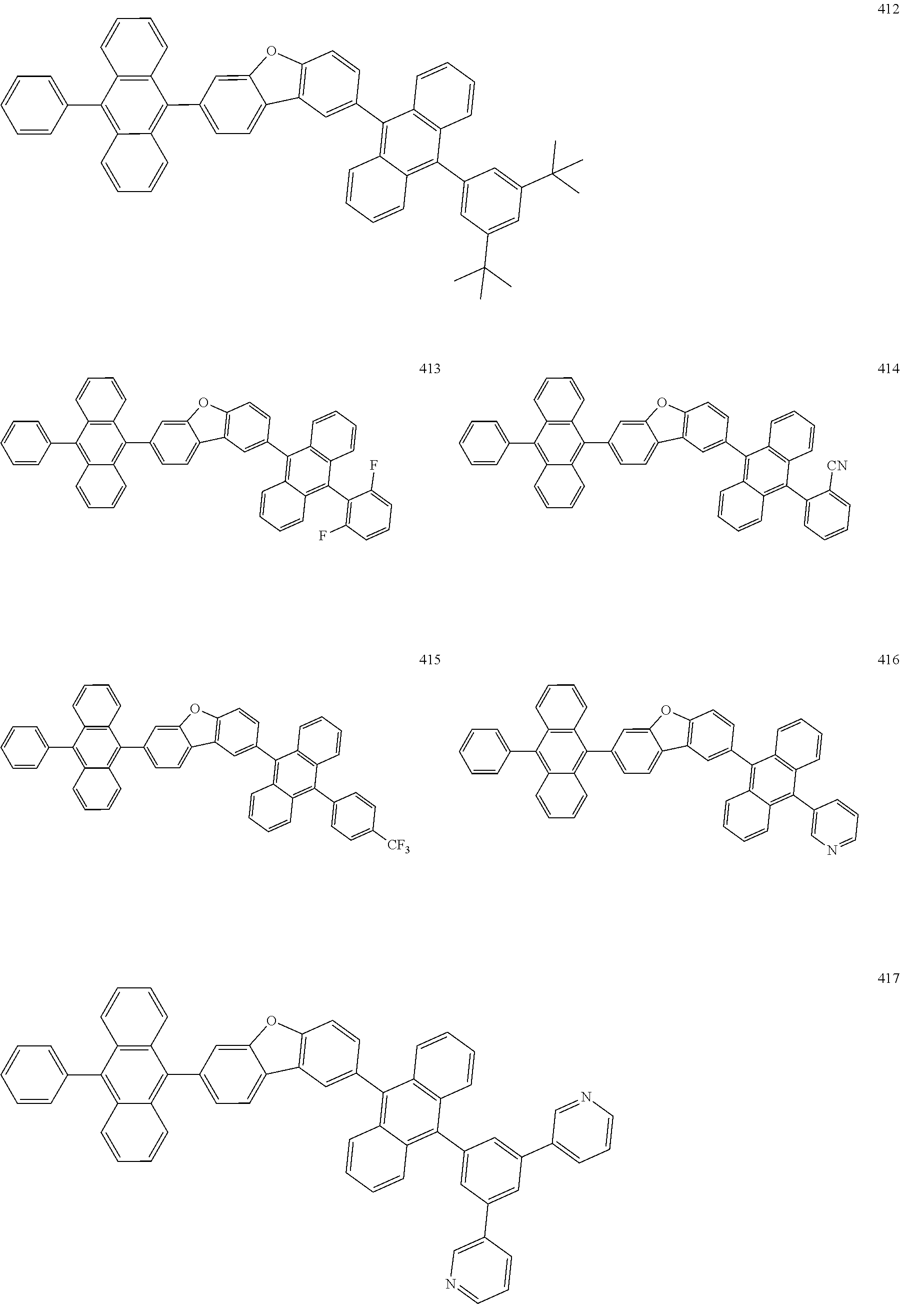

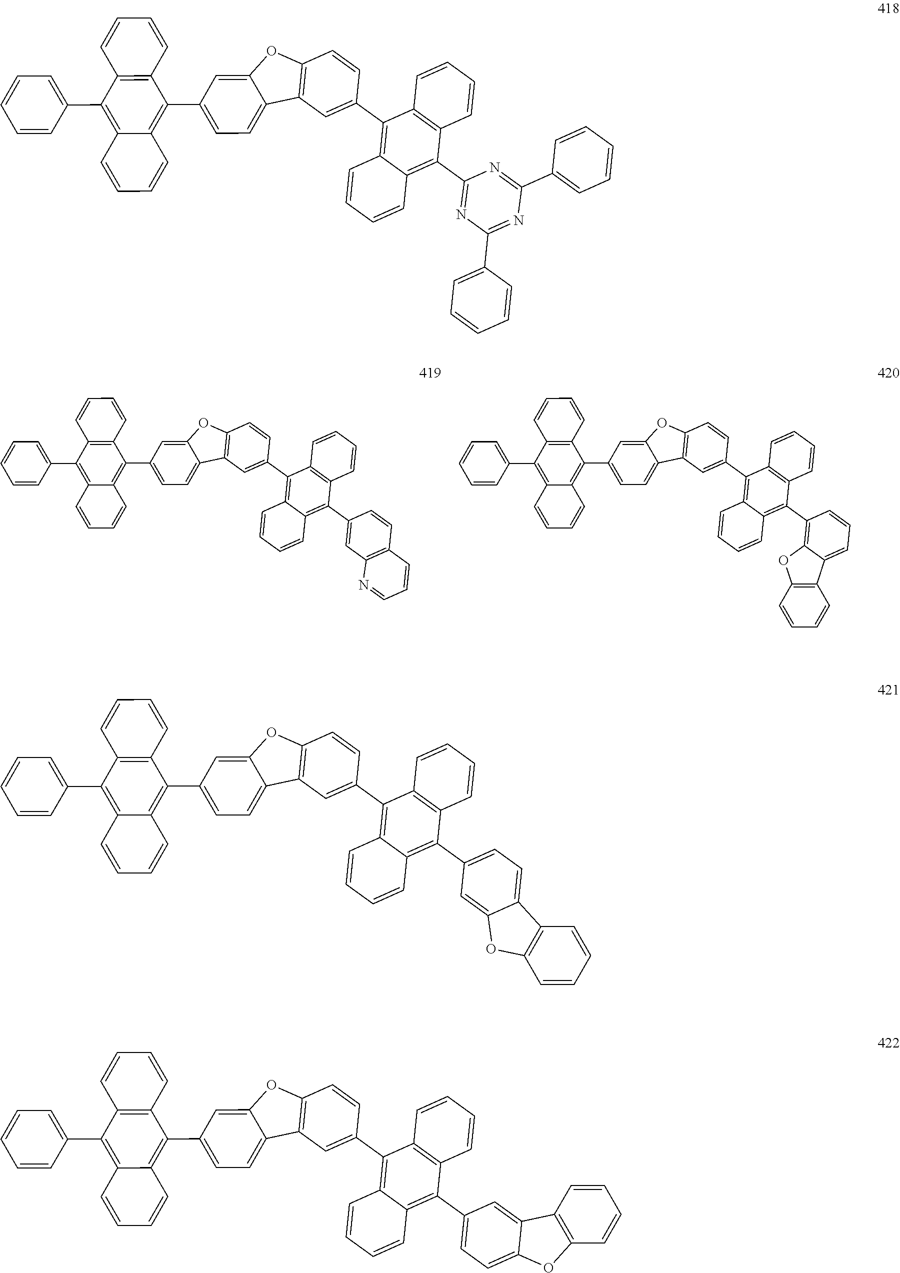

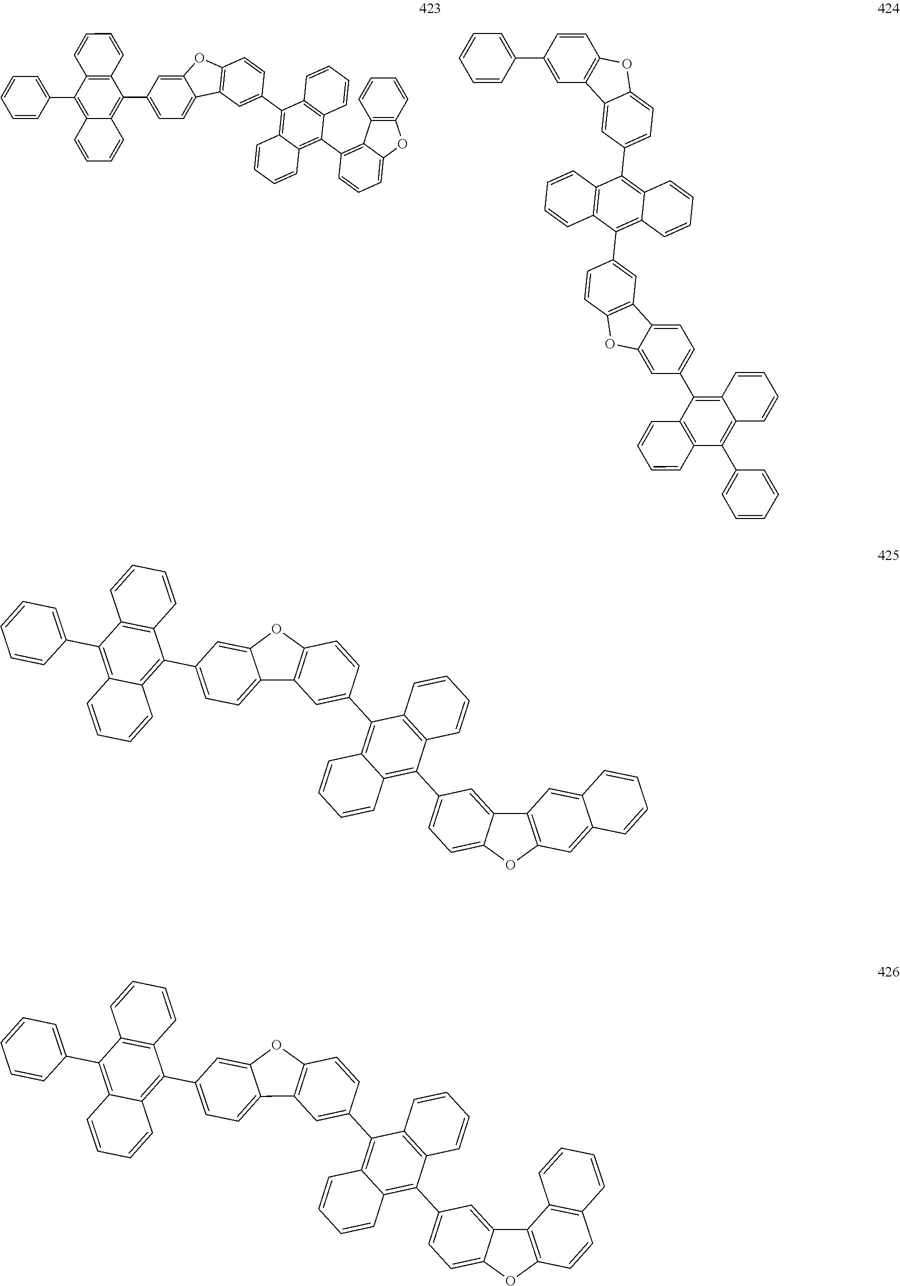 [SMILES: c1ccc(-c2c3ccccc3c(-c3ccc4c(c3)oc3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7oc8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)oc3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3ccc4c(c3)oc3ccc(-c5c6ccccc6c(-c6ccc7oc8ccc9ccccc9c8c7c6)c6ccccc56)cc34)c3ccccc23)cc1]

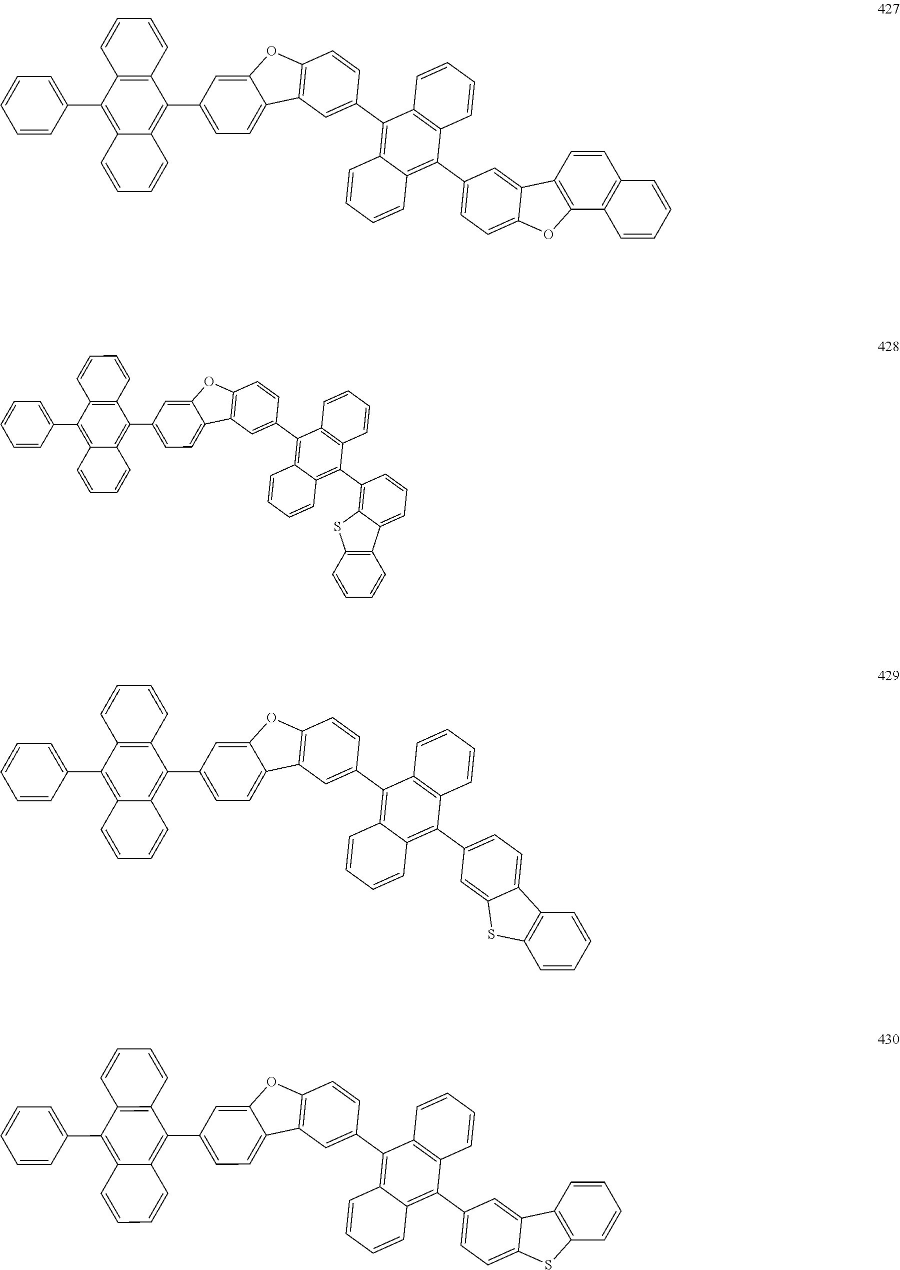 [SMILES: c1ccc(-c2c3ccccc3c(-c3ccc4c(c3)oc3ccc(-c5c6ccccc6c(-c6ccc7oc8c9ccccc9ccc8c7c6)c6ccccc56)cc34)c3ccccc23)cc1 ; c1ccc(-c2c3ccccc3c(-c3ccc4c(c3)oc3ccc(-c5c6ccccc6c(-c6cccc7c6sc6ccccc67)c6ccccc56)cc34)c3ccccc23)cc1 ; c1ccc(-c2c3ccccc3c(-c3ccc4c(c3)oc3ccc(-c5c6ccccc6c(-c6ccc7c(c6)sc6ccccc67)c6ccccc56)cc34)c3ccccc23)cc1 ; c1ccc(-c2c3ccccc3c(-c3ccc4c(c3)oc3ccc(-c5c6ccccc6c(-c6ccc7sc8ccccc8c7c6)c6ccccc56)cc34)c3ccccc23)cc1]

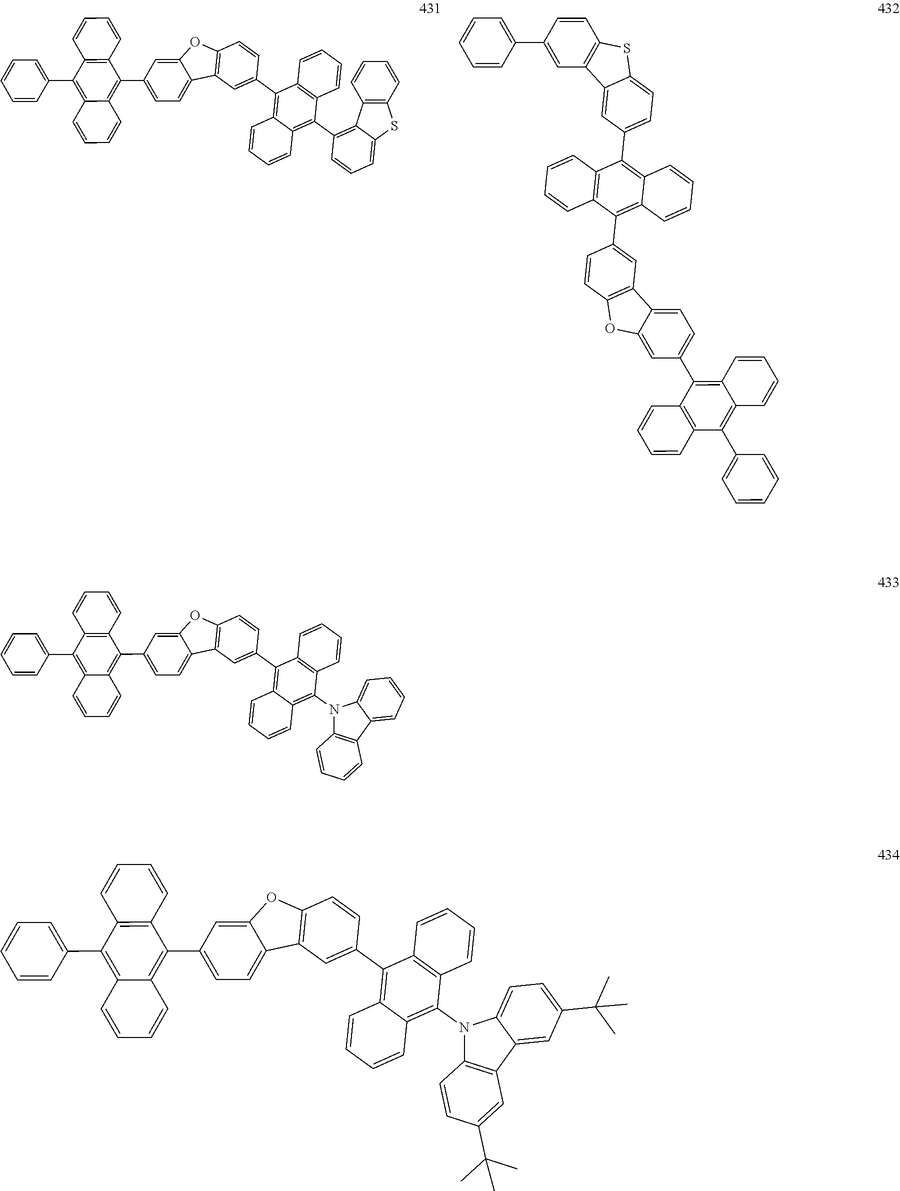 [SMILES: c1ccc(-c2c3ccccc3c(-c3ccc4c(c3)oc3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7oc8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)oc3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3oc4cc(-c5c6ccccc6c(-c6ccccc6)c6ccccc56)ccc4c3c2)c2ccccc12]

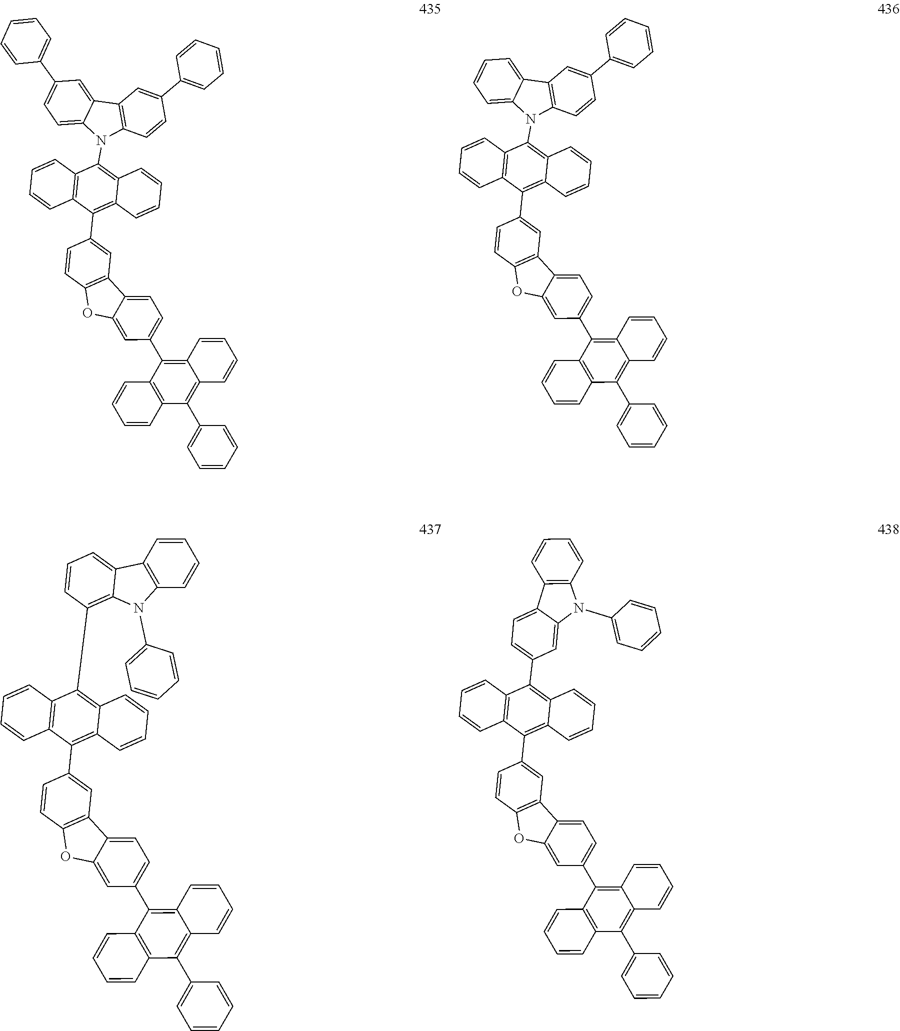 [SMILES: c1ccc(-c2ccc3c(c2)c2cc(-c4ccccc4)ccc2n3-c2c3ccccc3c(-c3ccc4oc5cc(-c6c7ccccc7c(-c7ccccc7)c7ccccc67)ccc5c4c3)c3ccccc23)cc1 ; c1ccc(-c2ccc3c(c2)c2ccccc2n3-c2c3ccccc3c(-c3ccc4oc5cc(-c6c7ccccc7c(-c7ccccc7)c7ccccc67)ccc5c4c3)c3ccccc23)cc1 ; c1ccc(-c2c3ccccc3c(-c3ccc4c(c3)oc3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)oc3ccc(-c5c6ccccc6c(-c6ccc7c8ccccc8n(-c8ccccc8)c7c6)c6ccccc56)cc34)c3ccccc23)cc1]

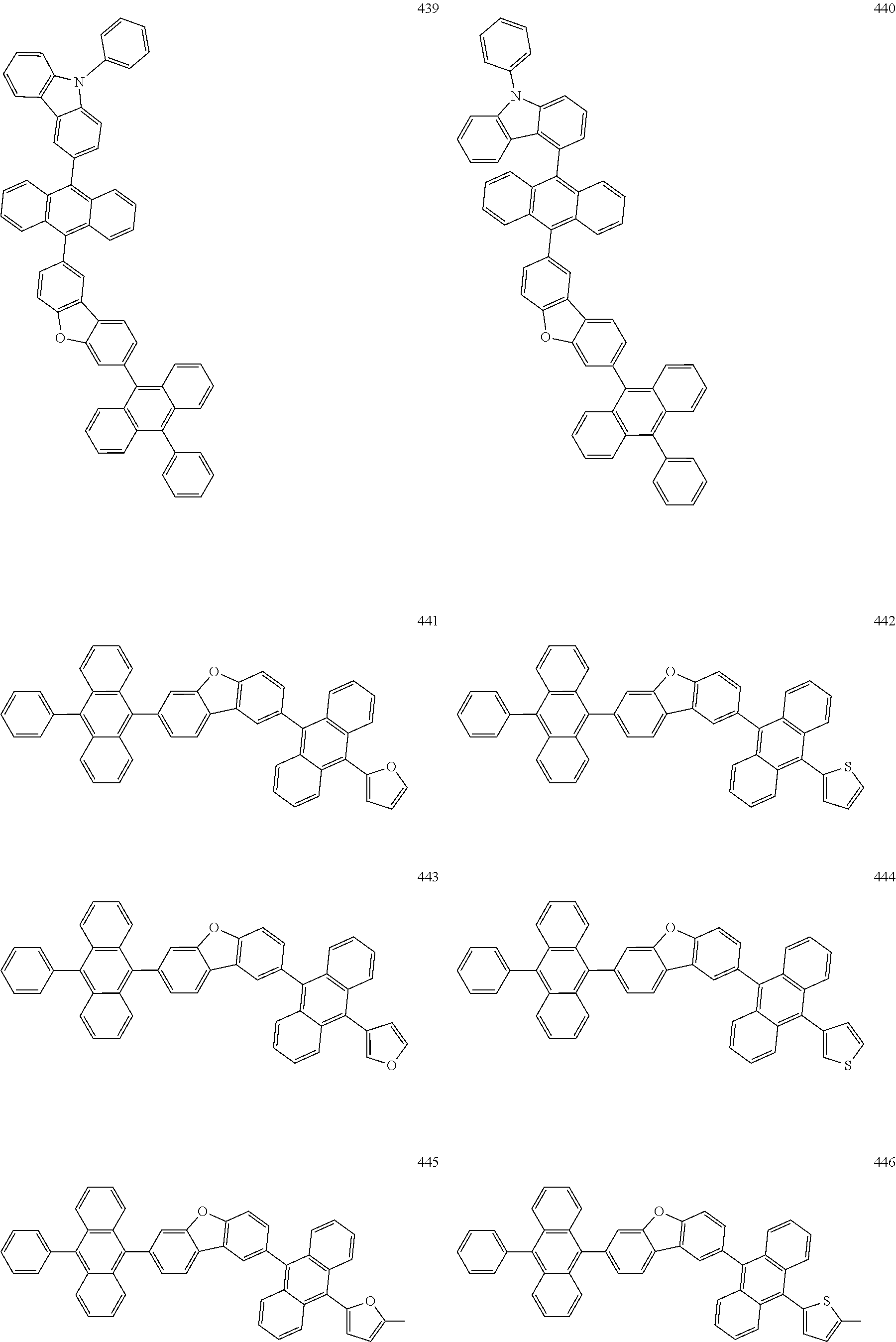

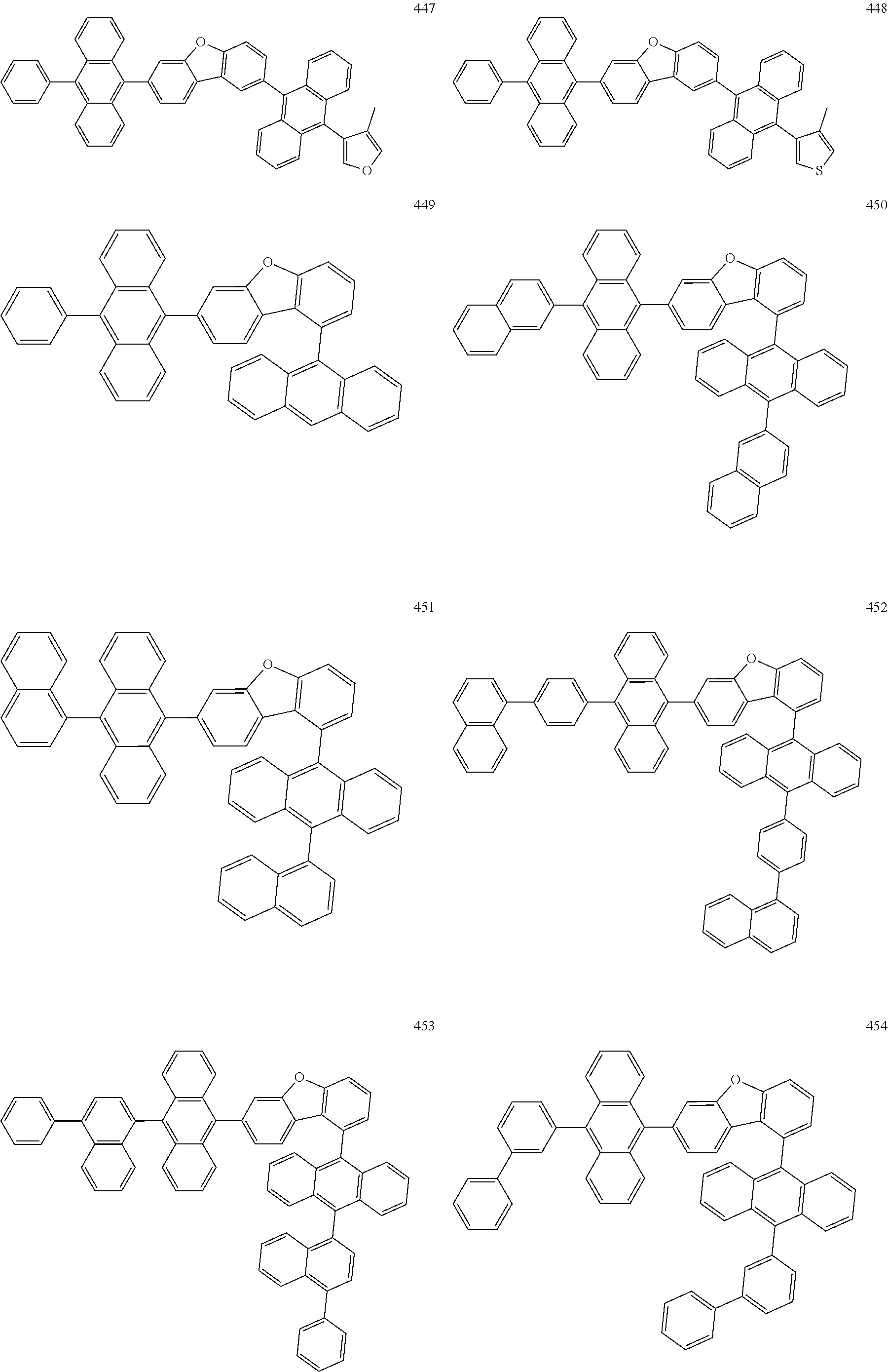

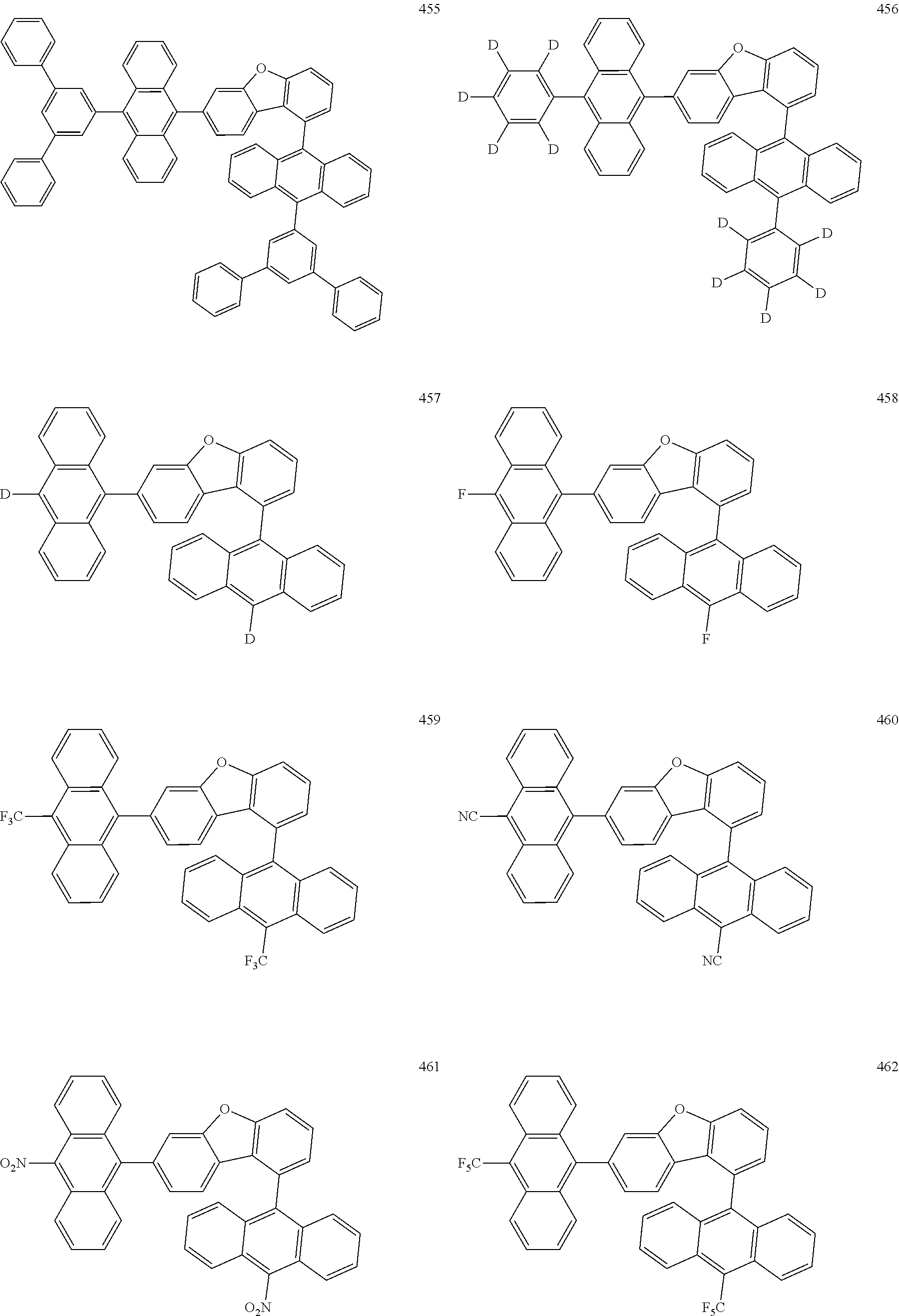

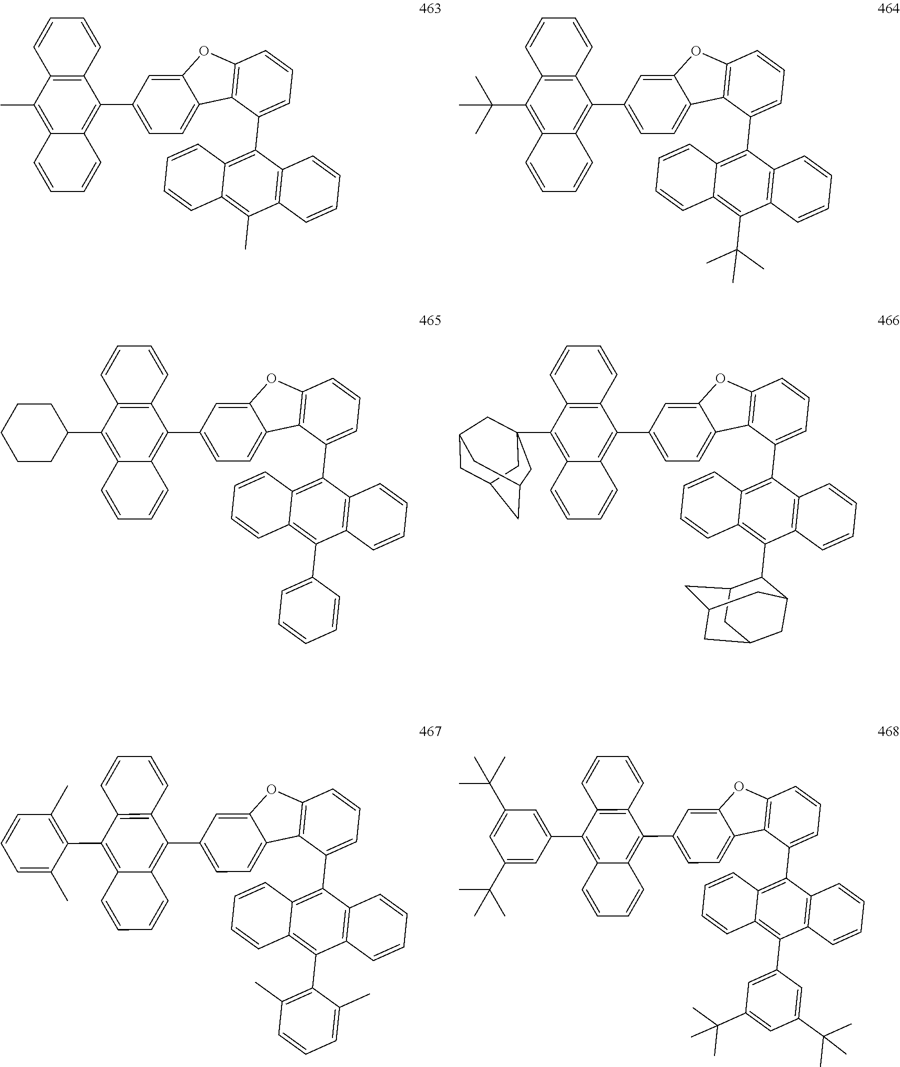

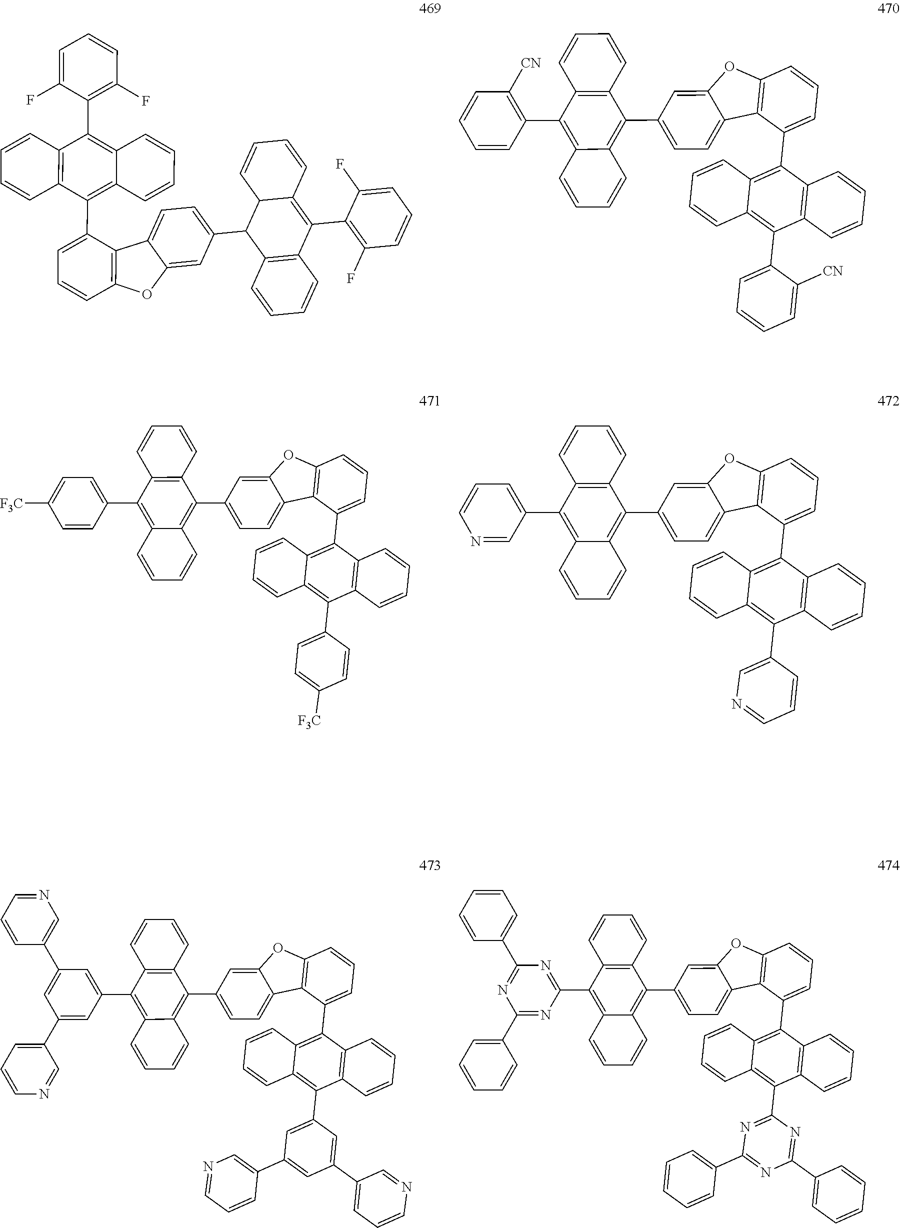

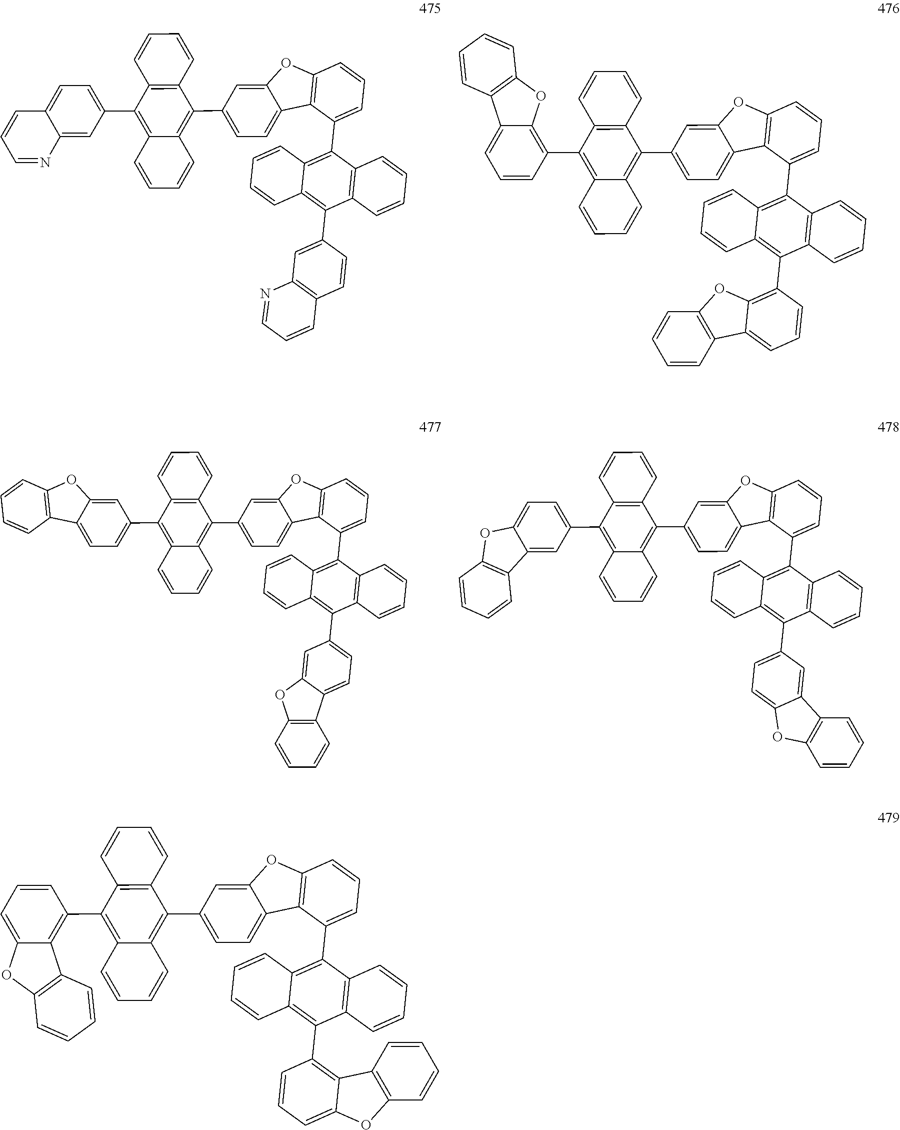

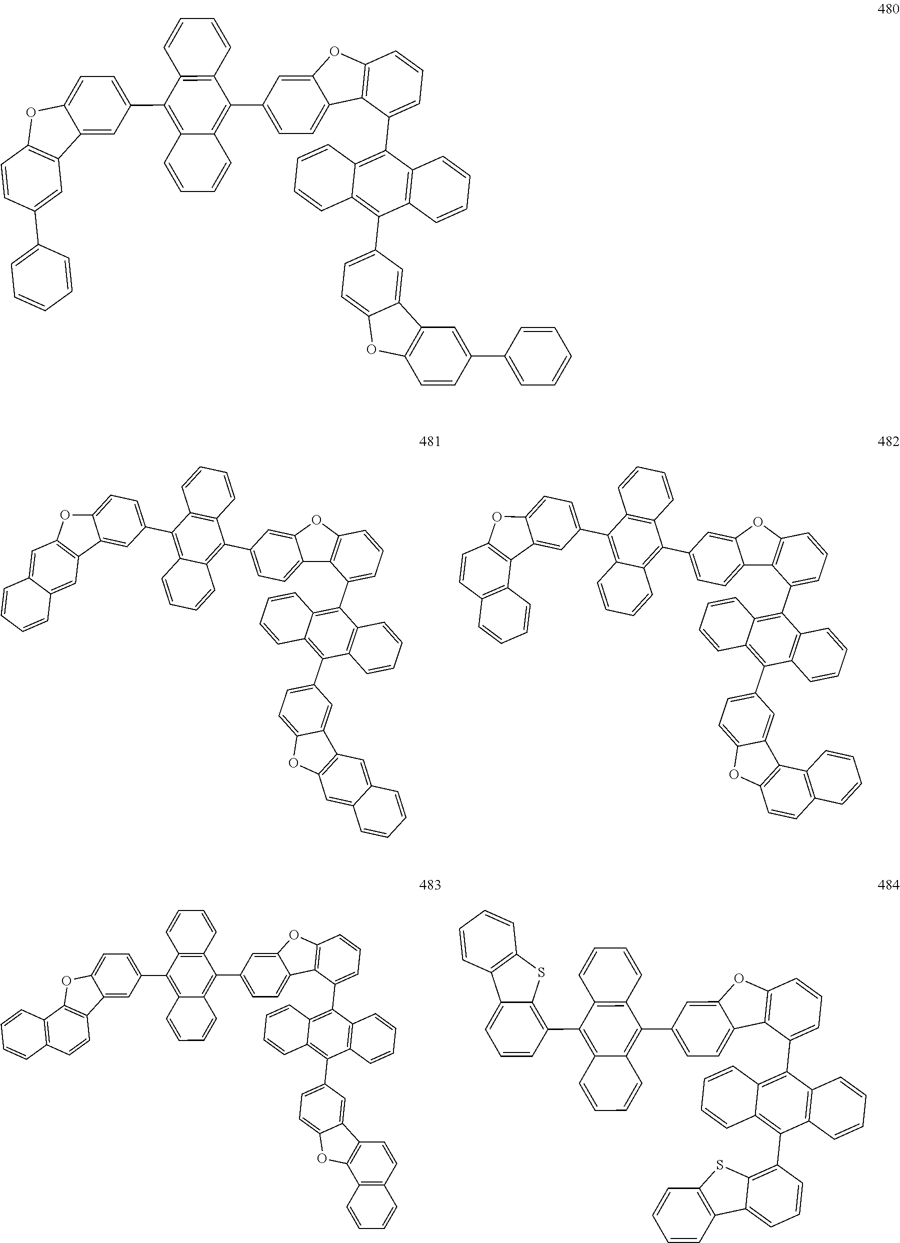

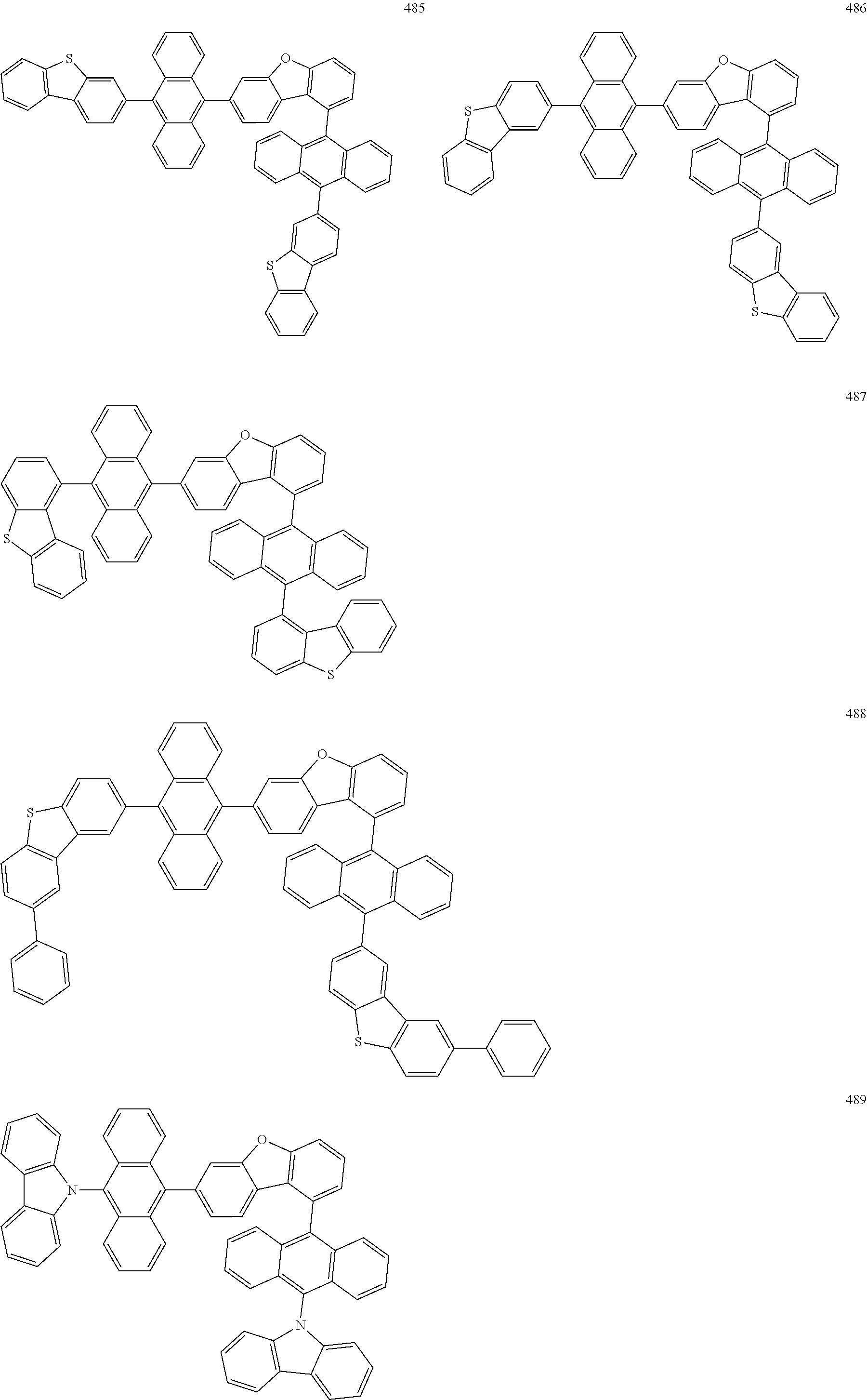

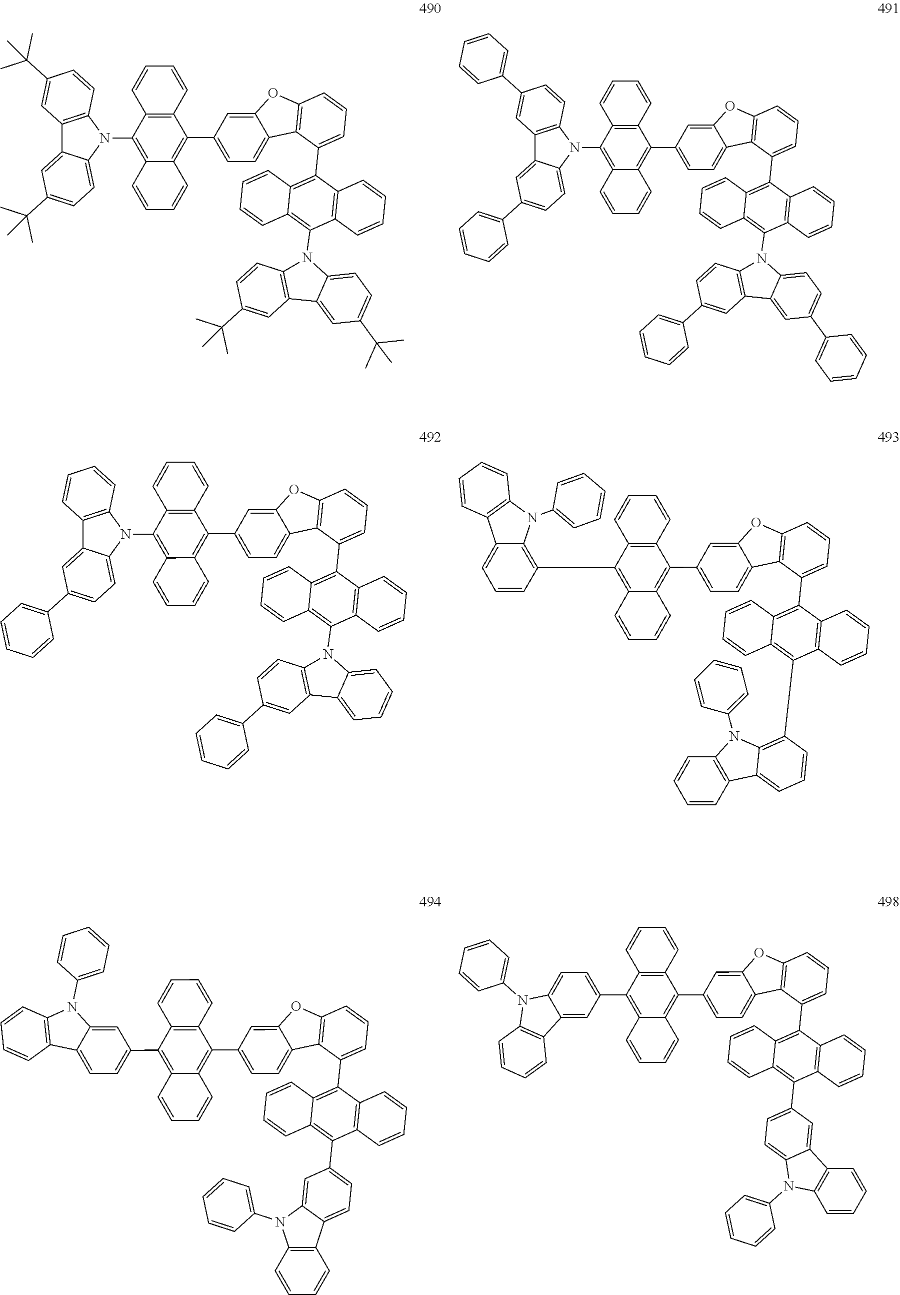

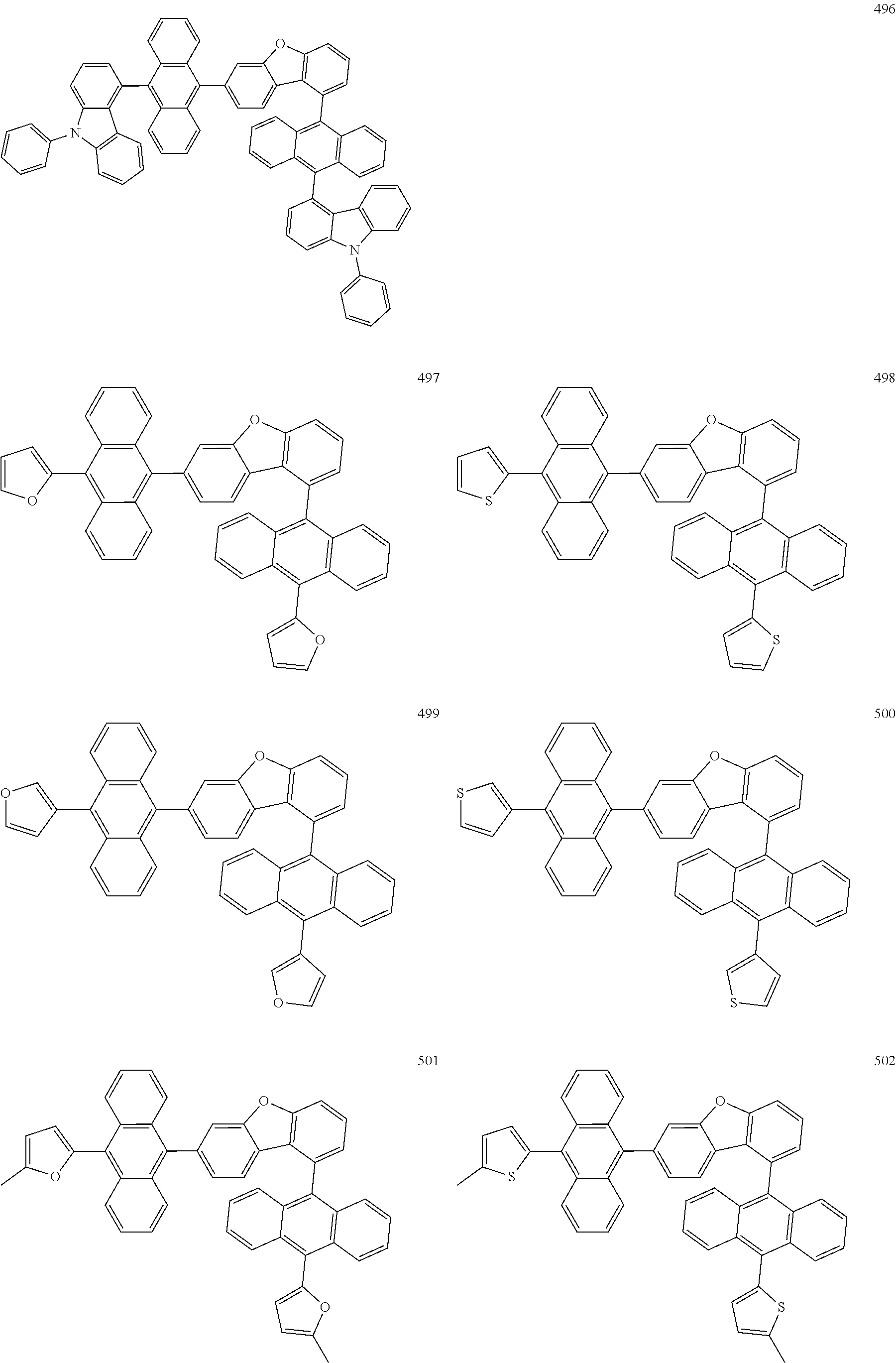

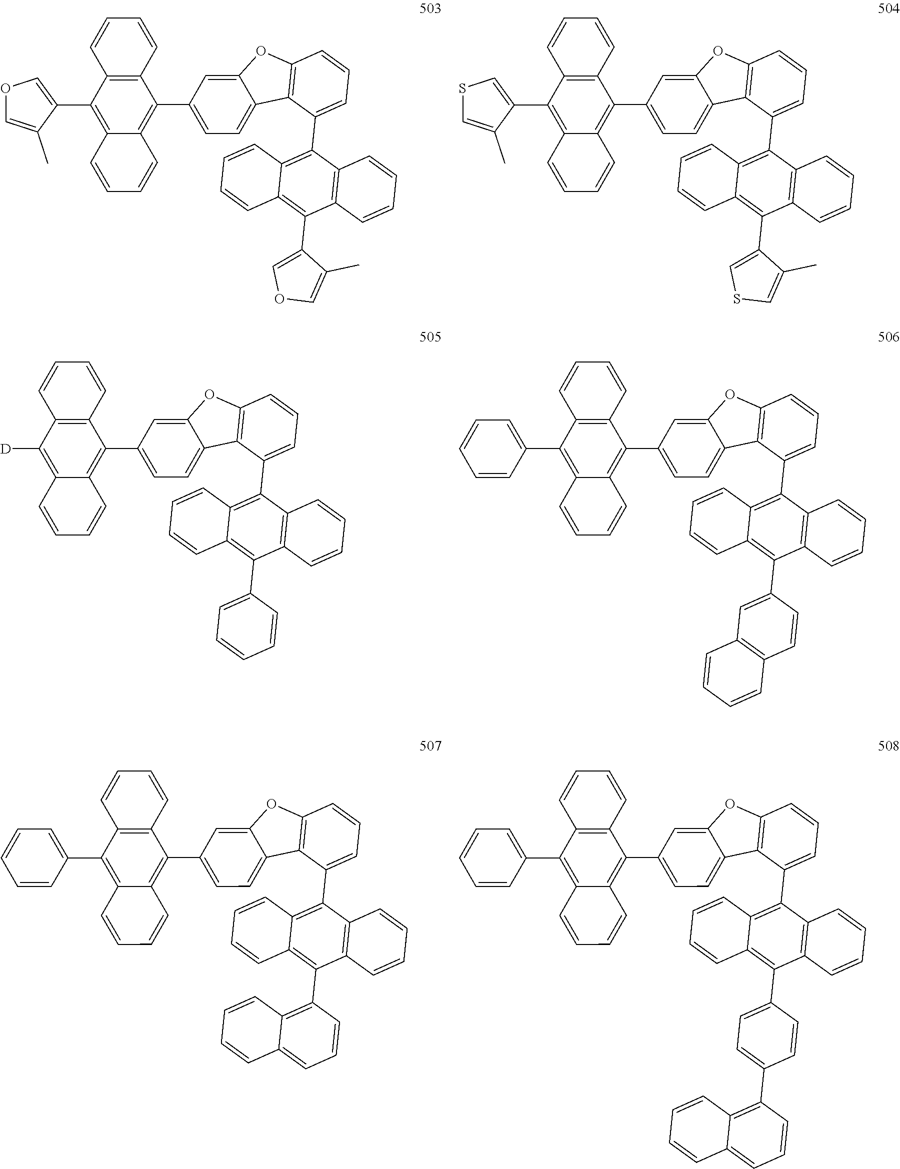

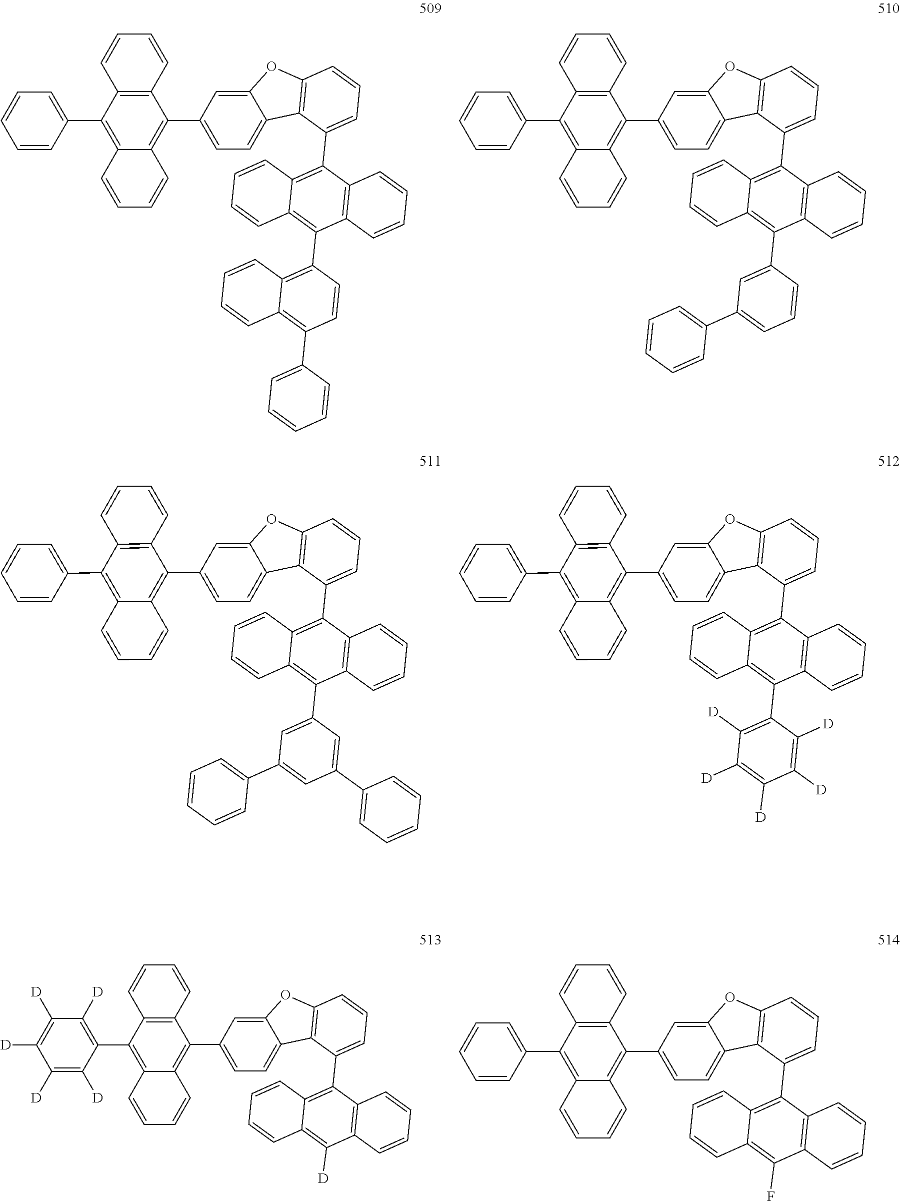

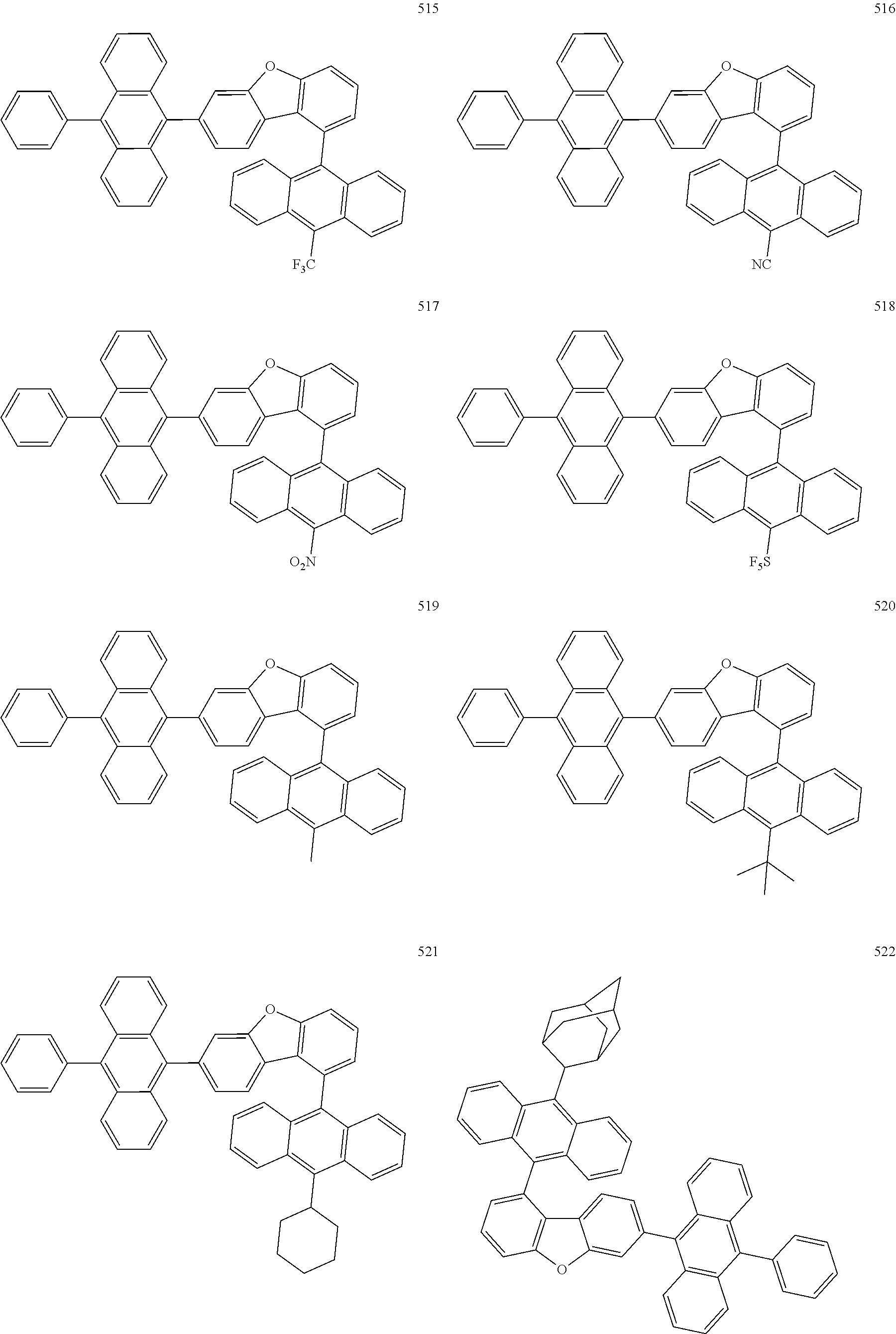

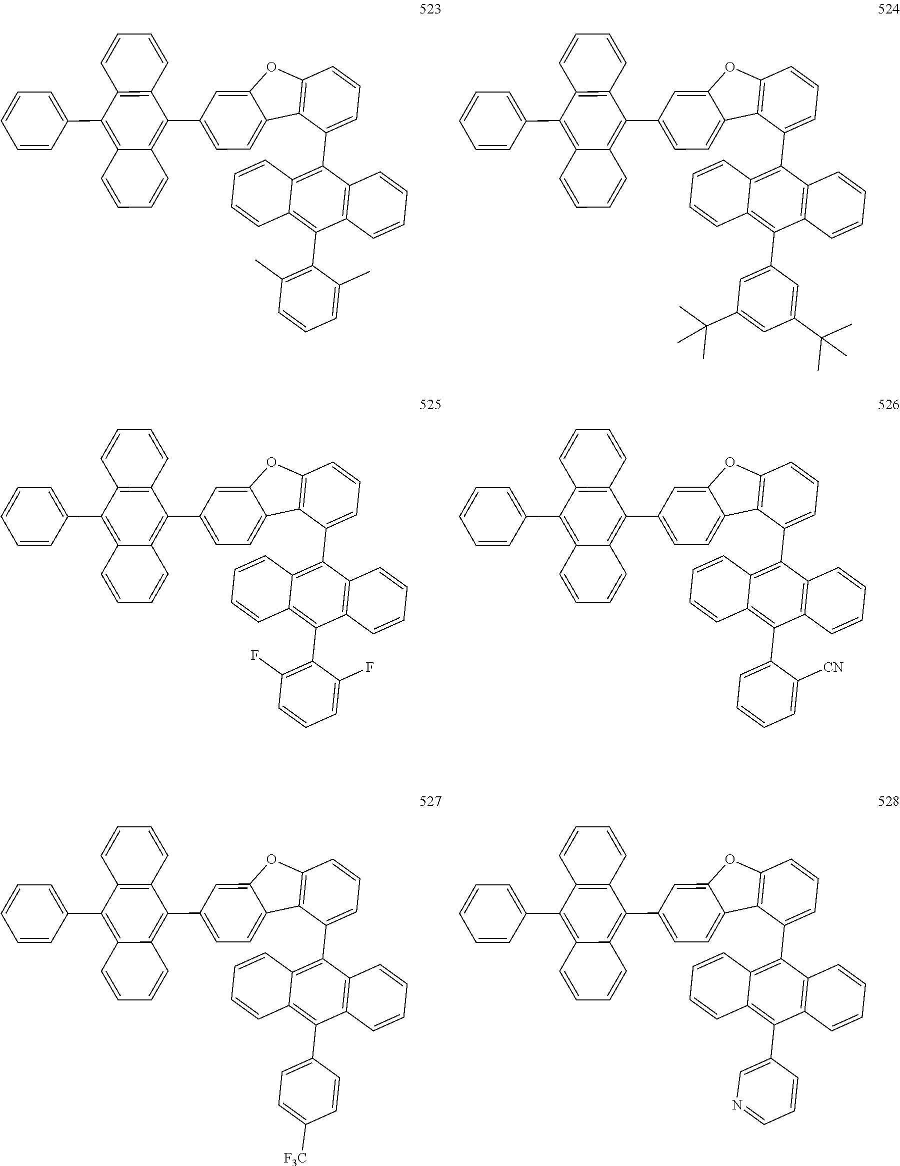

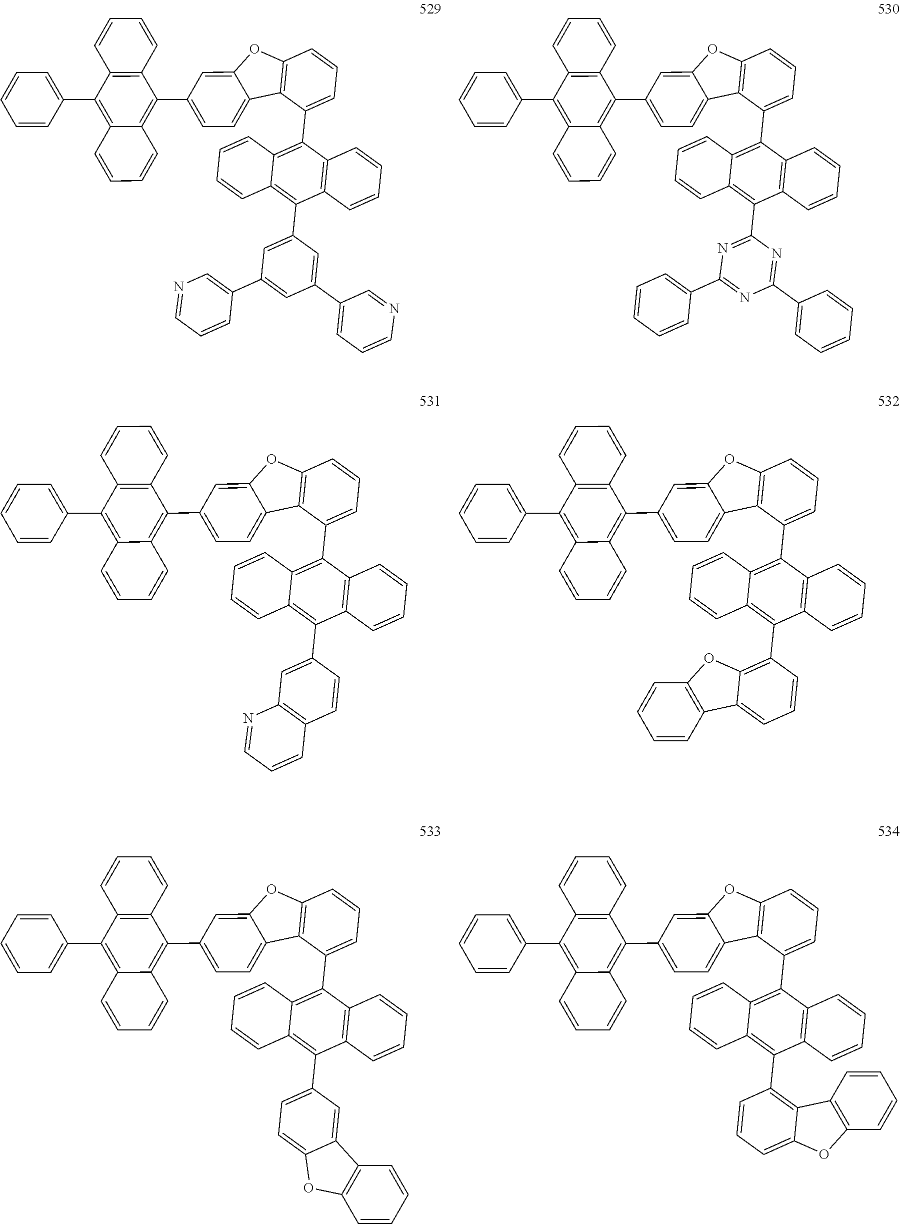

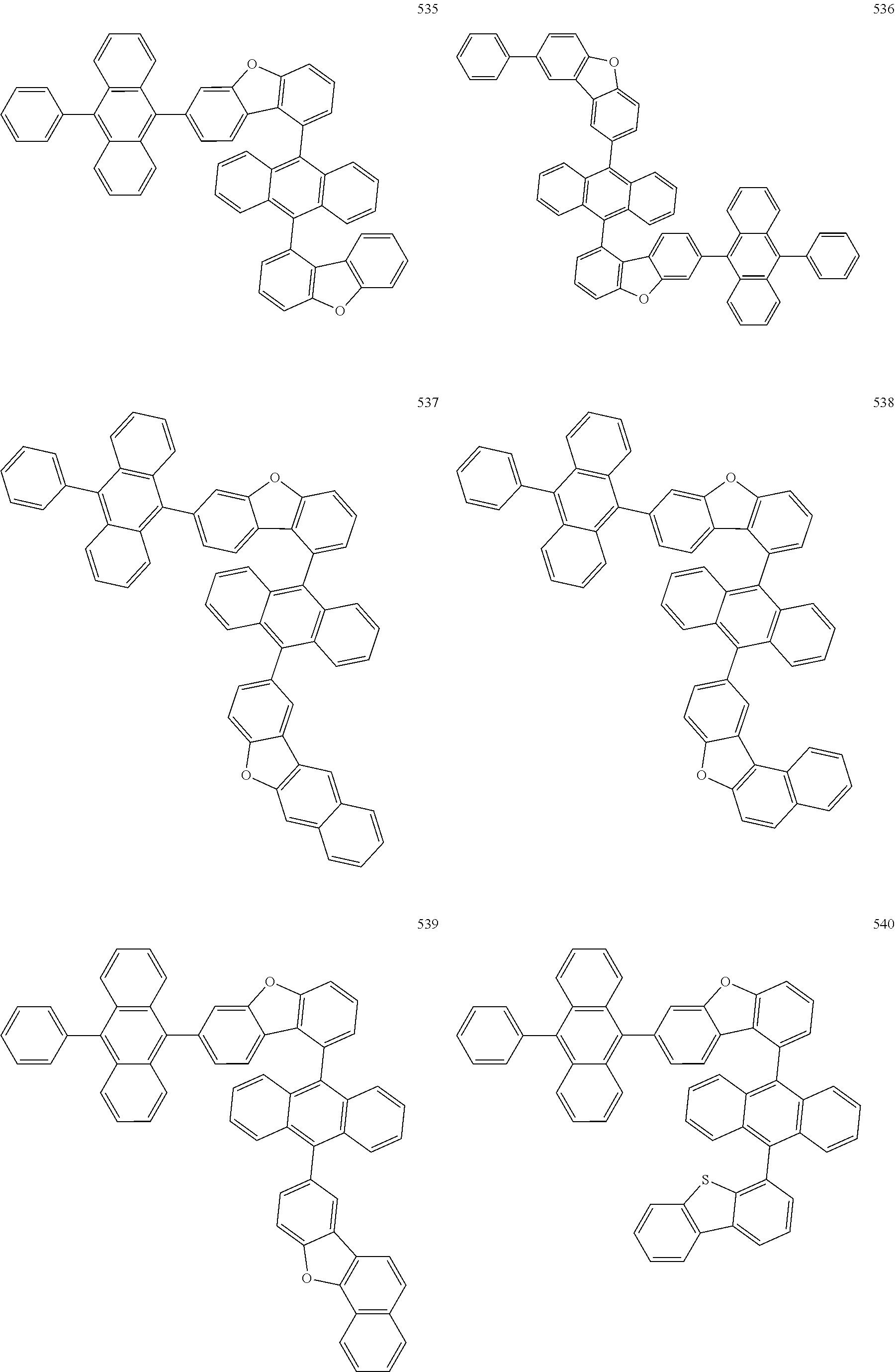

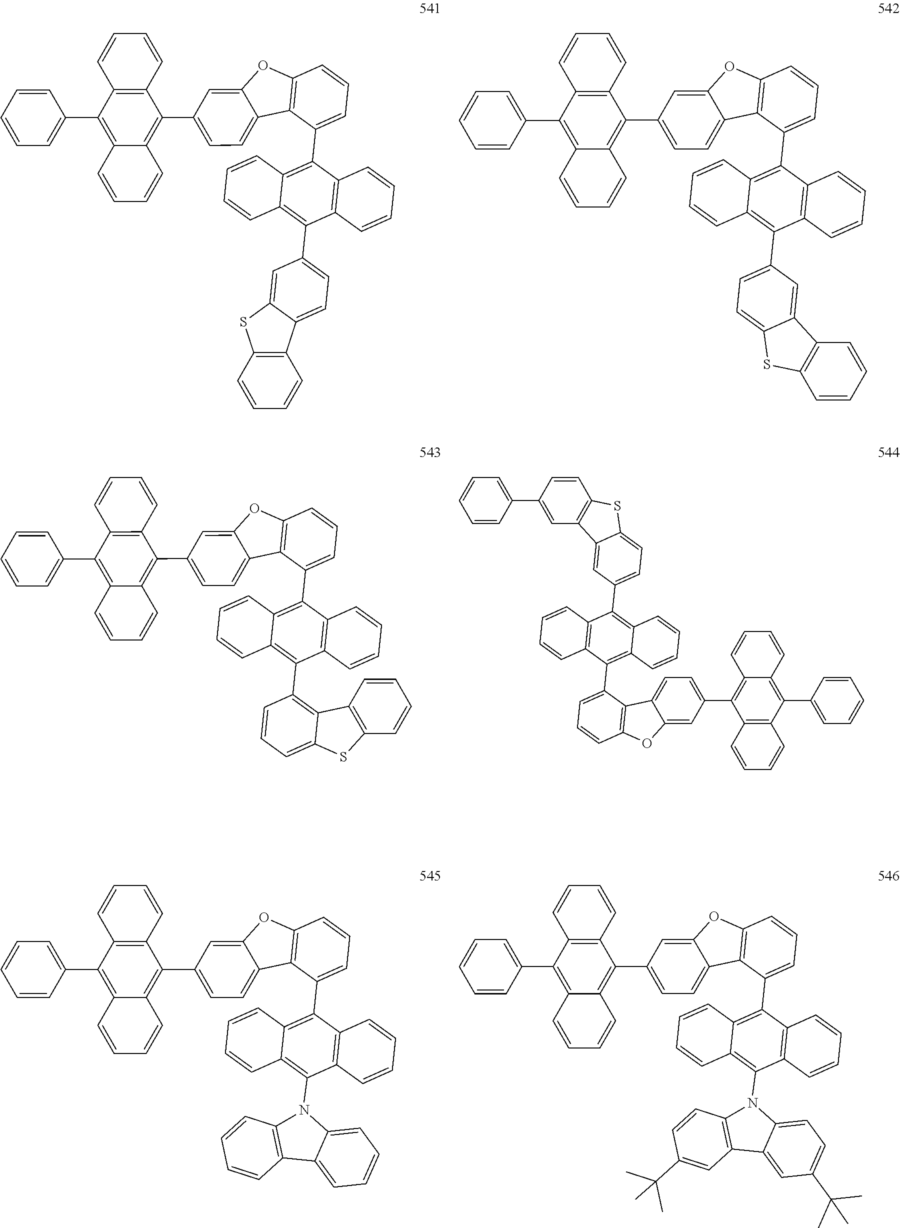

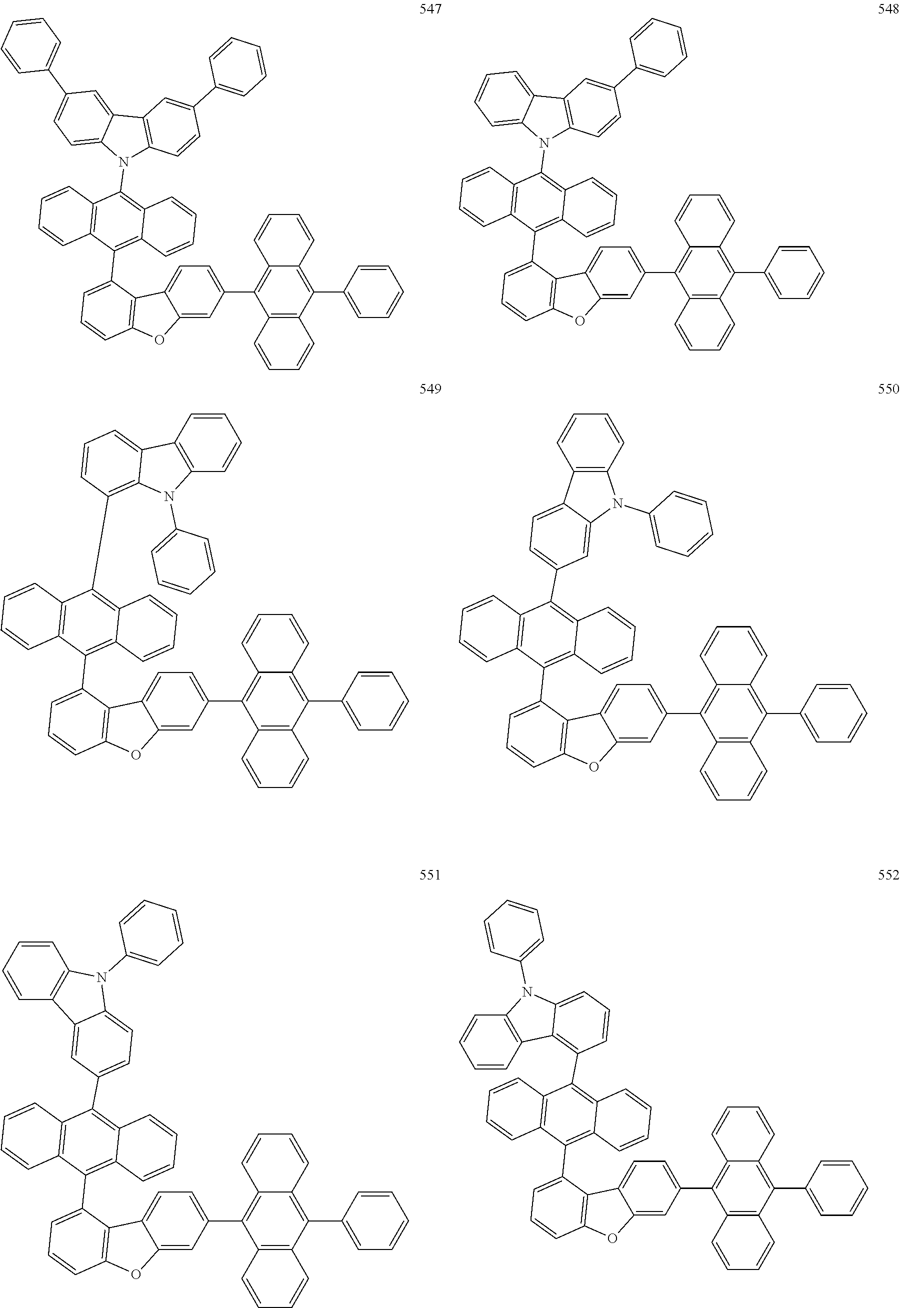

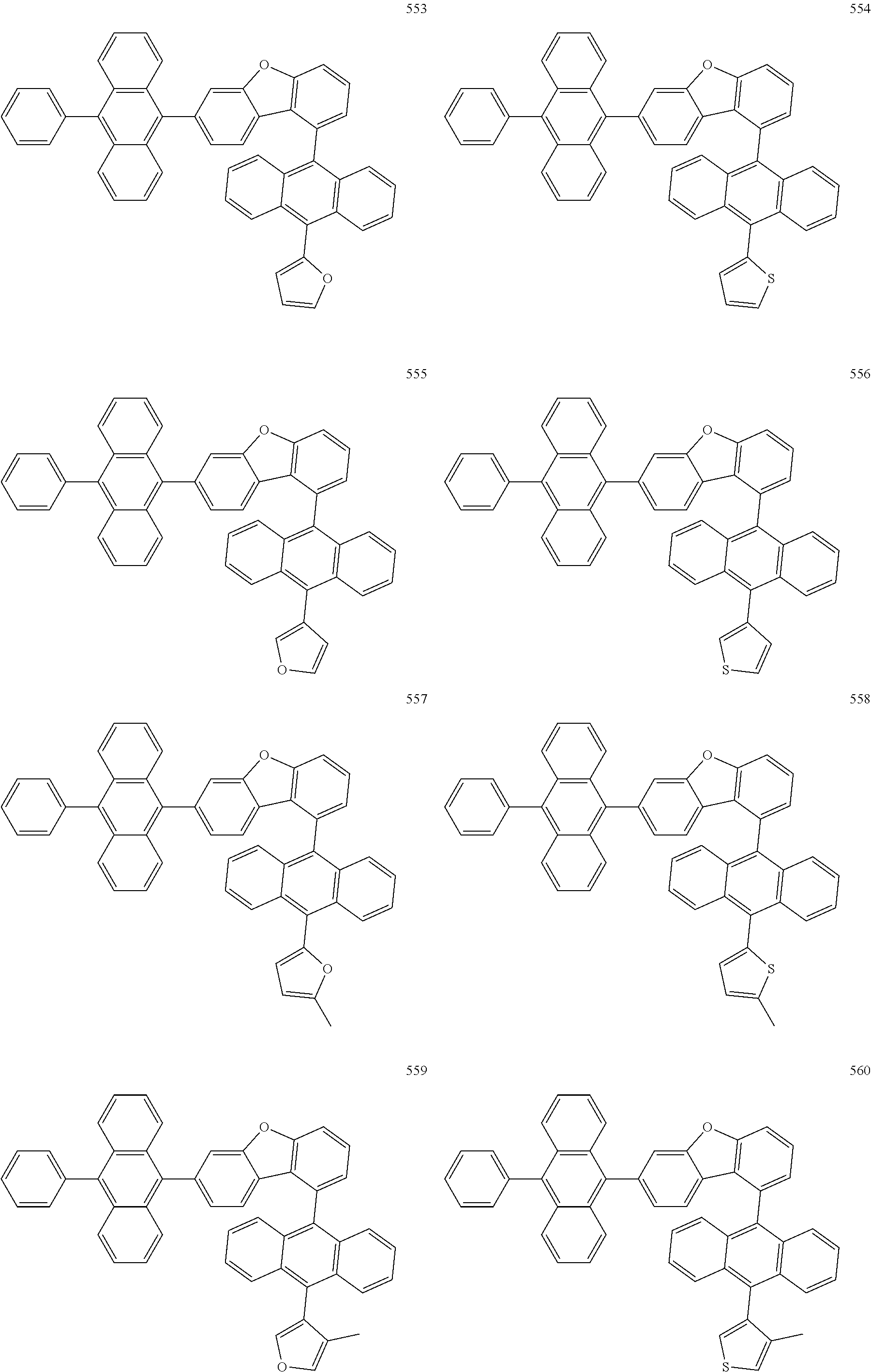

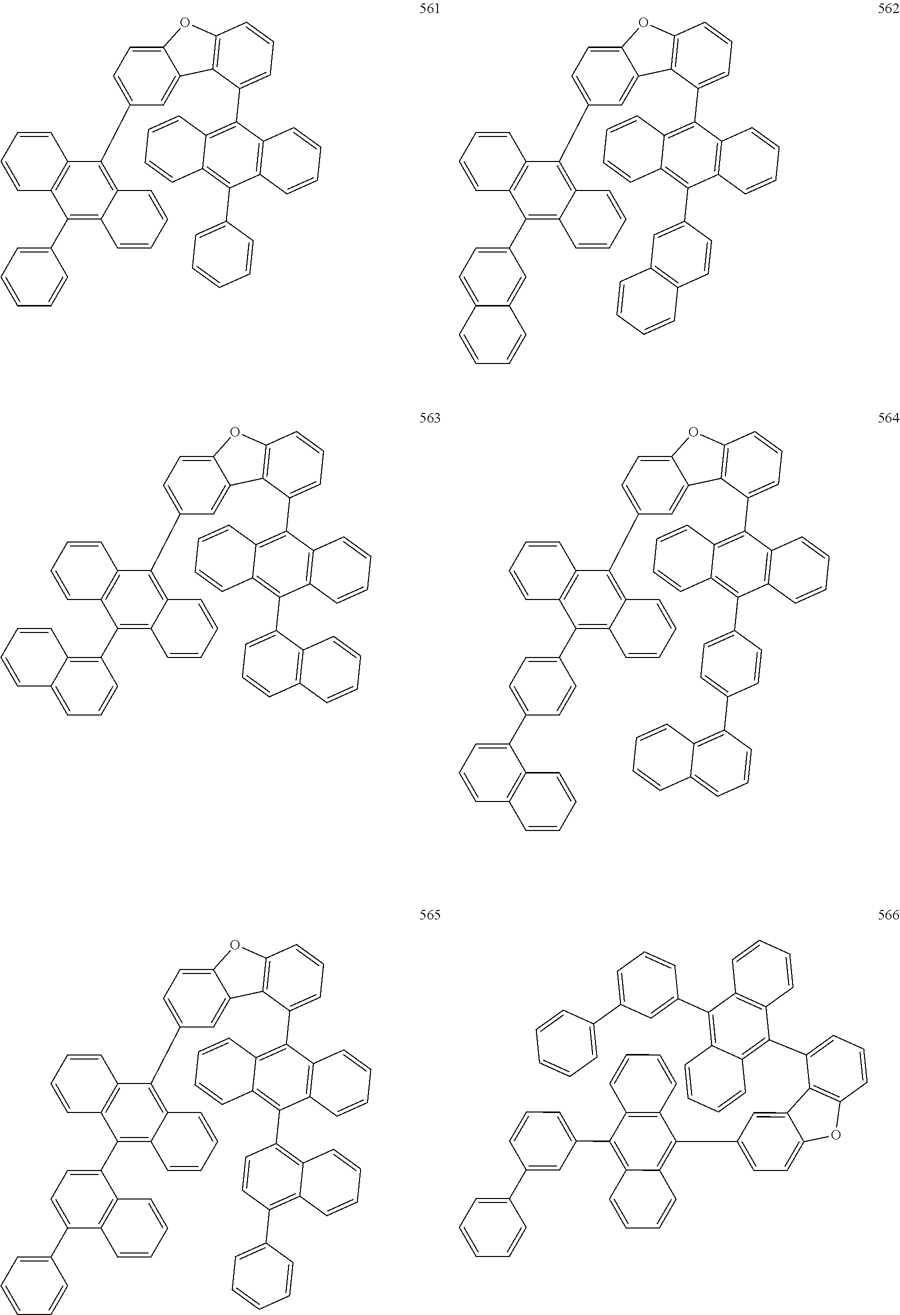

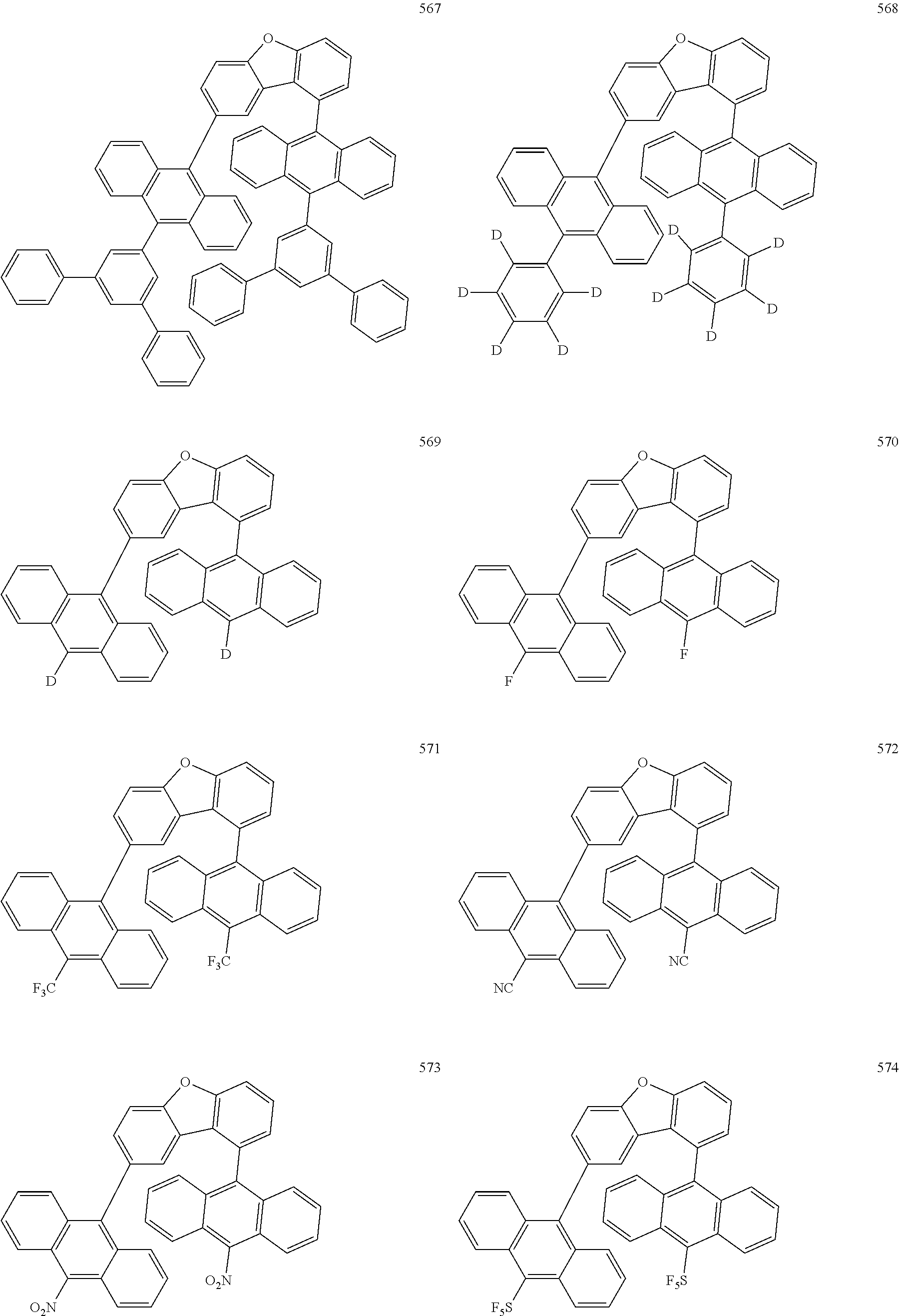

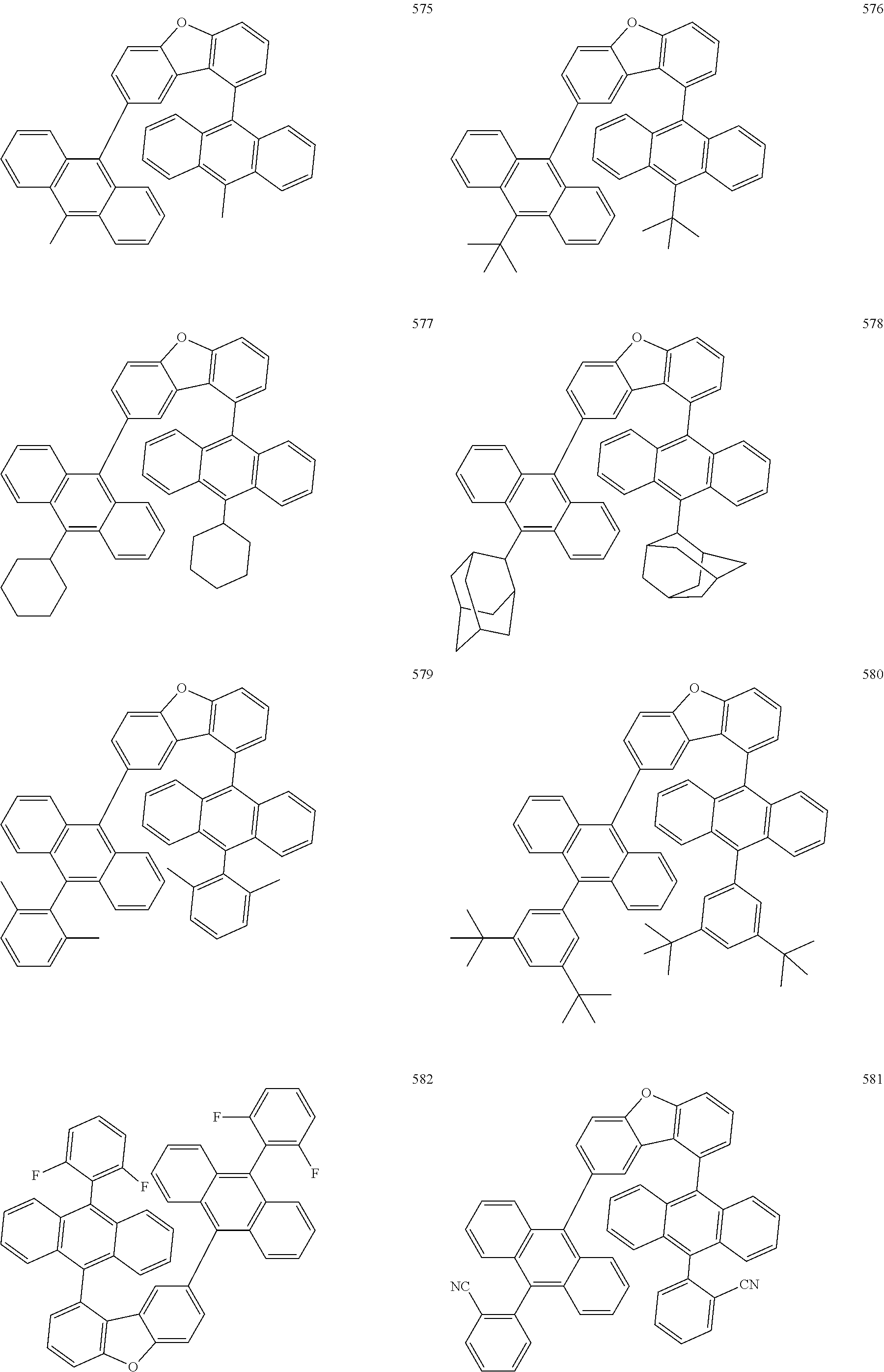

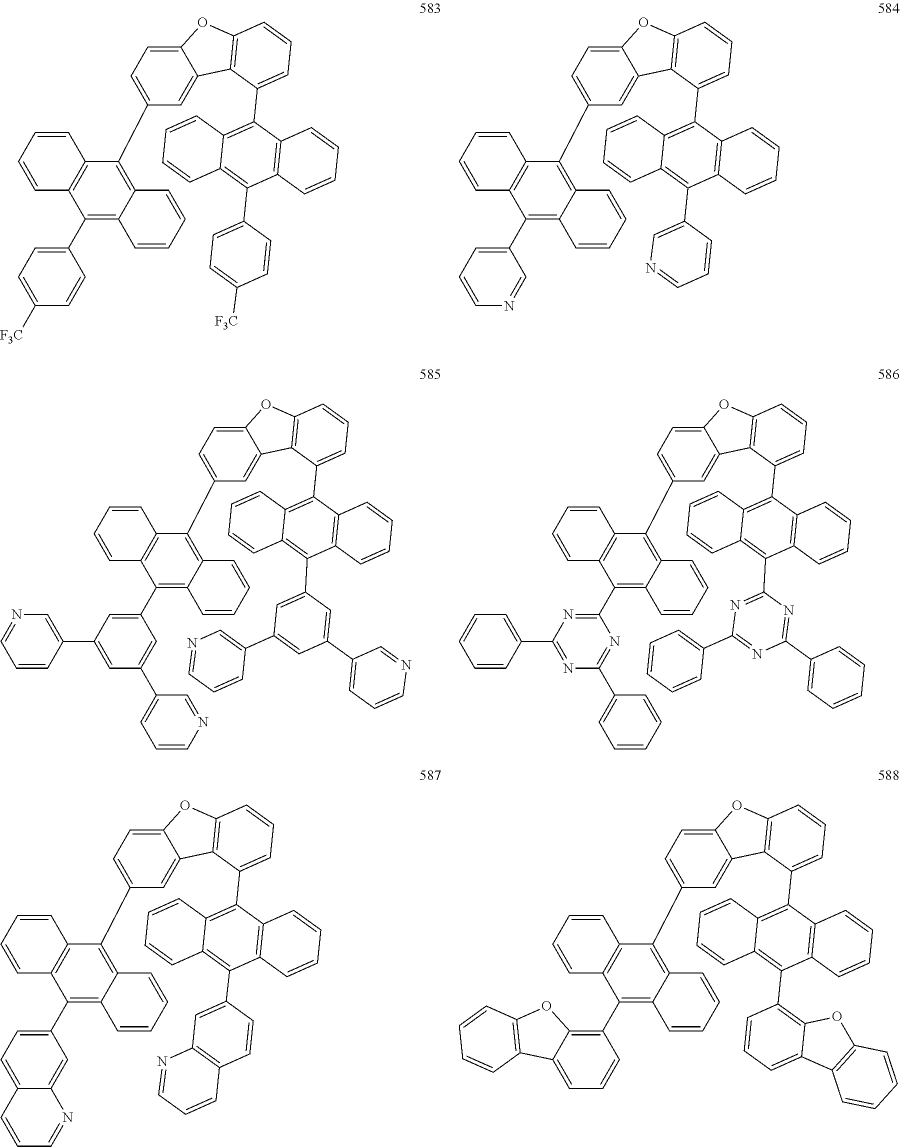

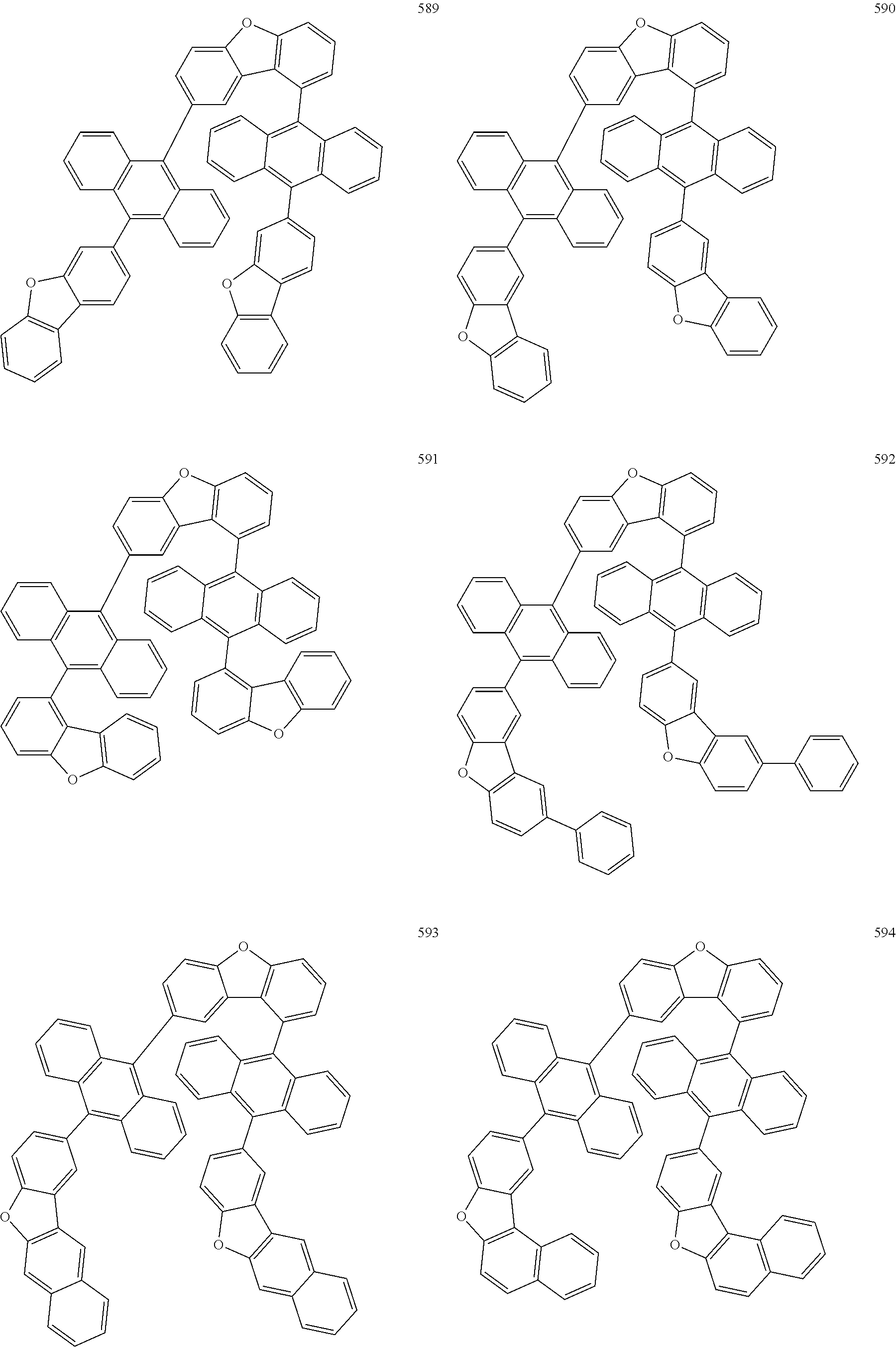

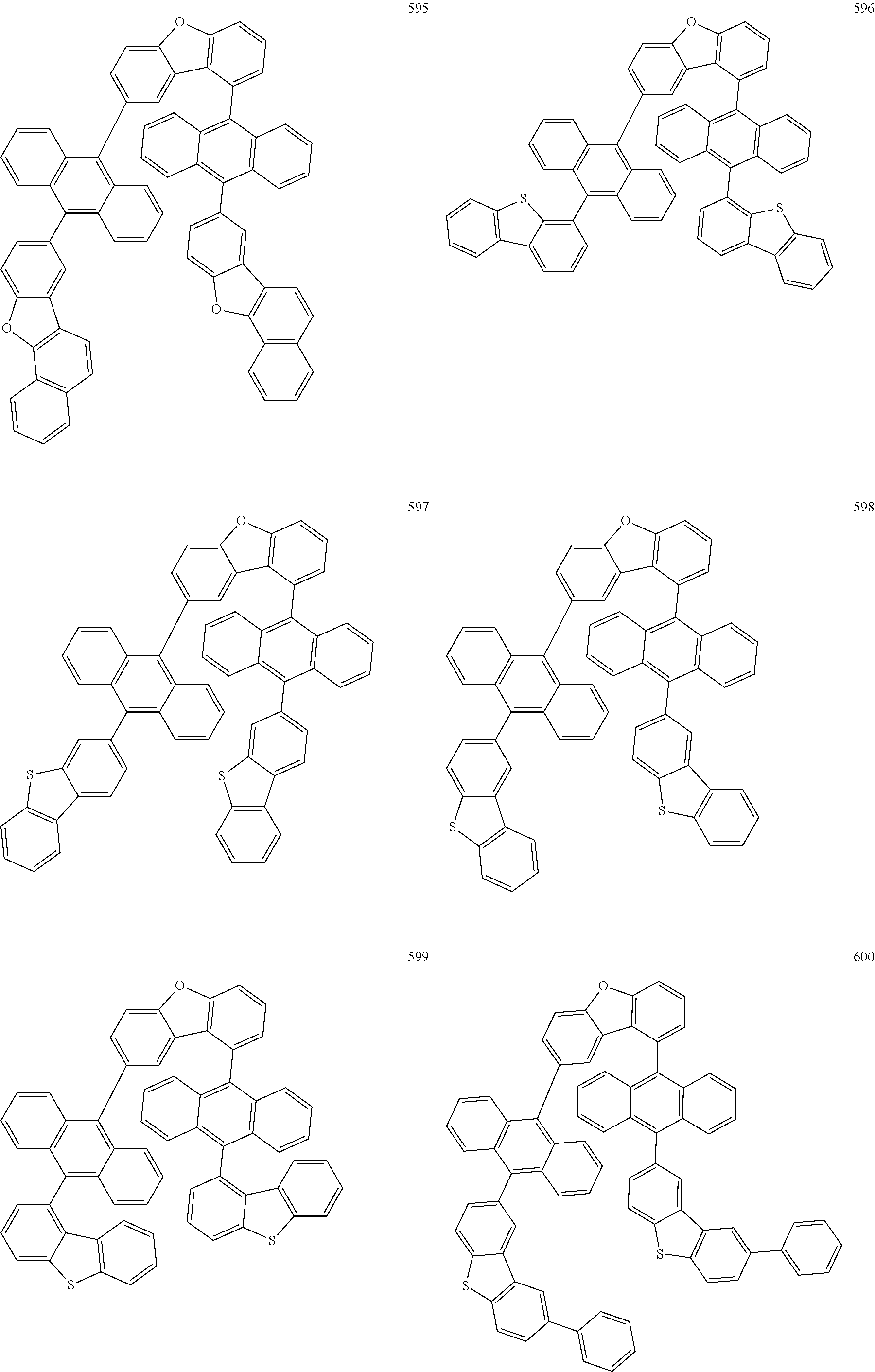

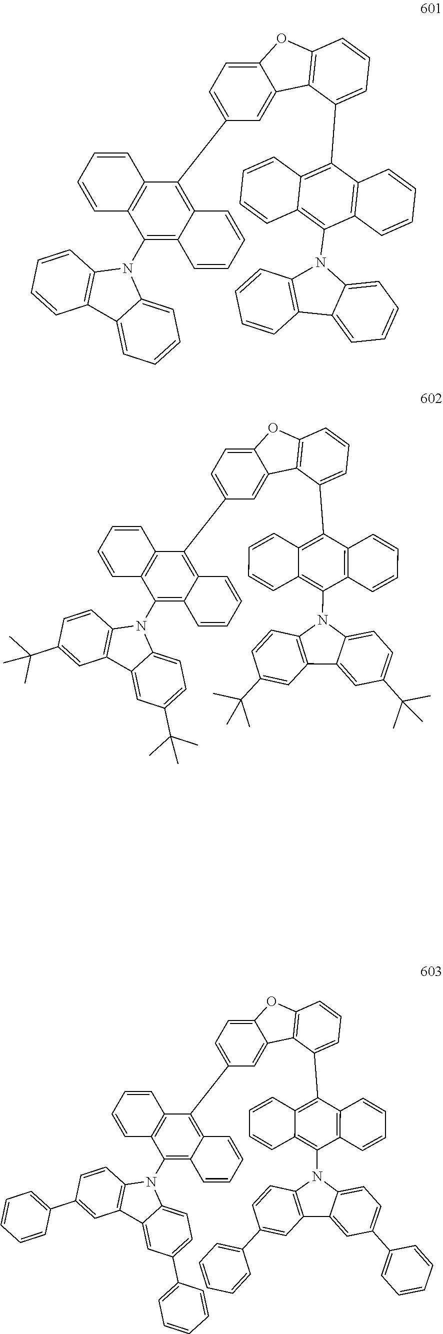 [SMILES: c1cc(-c2c3ccccc3c(-n3c4ccccc4c4ccccc43)c3ccccc23)c2c(c1)oc1ccc(-c3c4ccccc4c(-n4c5ccccc5c5ccccc54)c4ccccc34)cc12 ; CC(C)(C)c1ccc2c(c1)c1cc(C(C)(C)C)ccc1n2-c1c2ccccc2c(-c2ccc3oc4cccc(-c5c6ccccc6c(-n6c7ccc(C(C)(C)C)cc7c7cc(C(C)(C)C)ccc76)c6ccccc56)c4c3c2)c2ccccc12 ; c1ccc(-c2ccc3c(c2)c2cc(-c4ccccc4)ccc2n3-c2c3ccccc3c(-c3ccc4oc5cccc(-c6c7ccccc7c(-n7c8ccc(-c9ccccc9)cc8c8cc(-c9ccccc9)ccc87)c7ccccc67)c5c4c3)c3ccccc23)cc1]

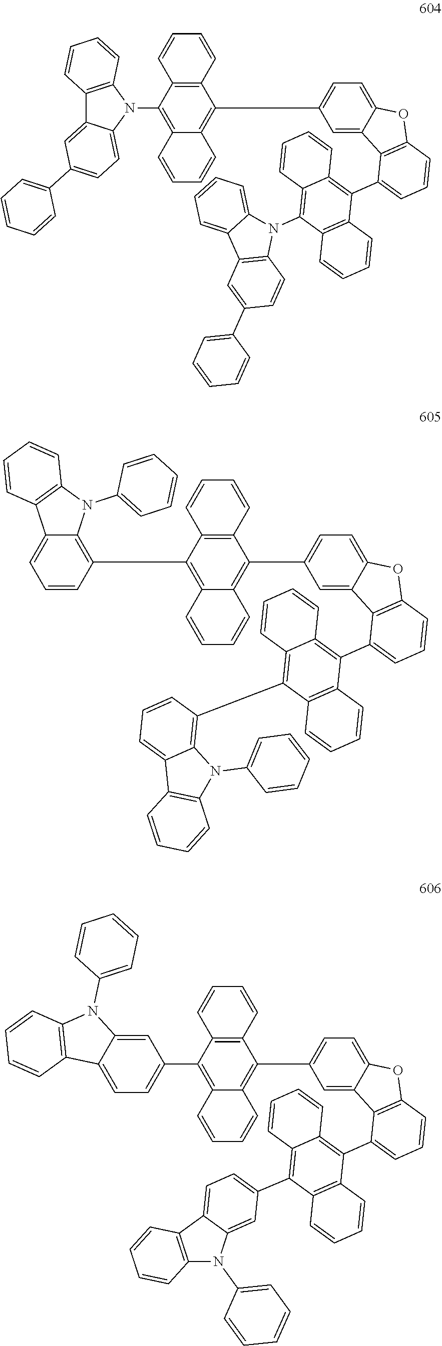 [SMILES: c1ccc(-c2ccc3c(c2)c2ccccc2n3-c2c3ccccc3c(-c3ccc4oc5cccc(-c6c7ccccc7c(-n7c8ccccc8c8cc(-c9ccccc9)ccc87)c7ccccc67)c5c4c3)c3ccccc23)cc1 ; c1ccc(-n2c3ccccc3c3cccc(-c4c5ccccc5c(-c5ccc6oc7cccc(-c8c9ccccc9c(-c9cccc%10c%11ccccc%11n(-c%11ccccc%11)c9%10)c9ccccc89)c7c6c5)c5ccccc45)c32)cc1 ; c1ccc(-n2c3ccccc3c3ccc(-c4c5ccccc5c(-c5ccc6oc7cccc(-c8c9ccccc9c(-c9ccc%10c%11ccccc%11n(-c%11ccccc%11)c%10c9)c9ccccc89)c7c6c5)c5ccccc45)cc32)cc1]

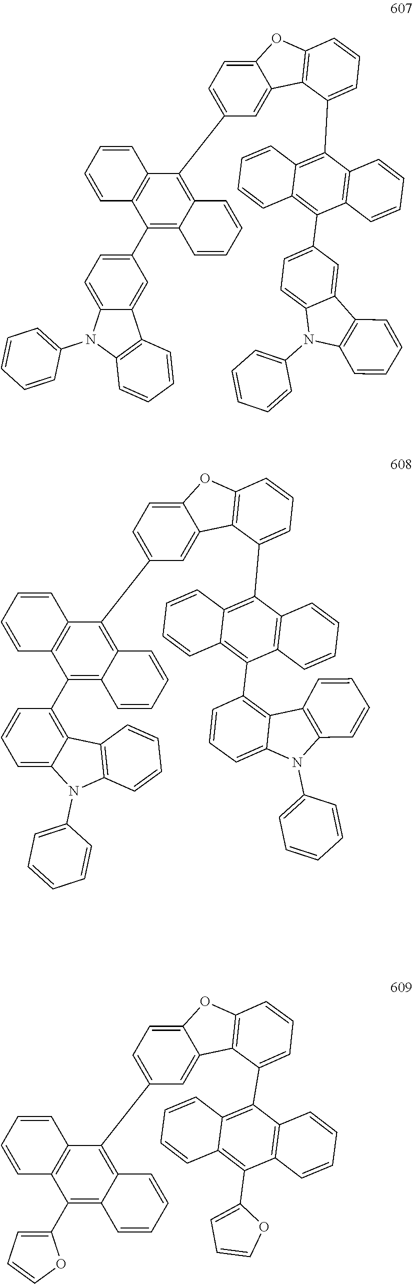 [SMILES: c1ccc(-n2c3ccccc3c3cc(-c4c5ccccc5c(-c5ccc6oc7cccc(-c8c9ccccc9c(-c9ccc%10c(c9)c9ccccc9n%10-c9ccccc9)c9ccccc89)c7c6c5)c5ccccc45)ccc32)cc1 ; c1ccc(-n2c3ccccc3c3c(-c4c5ccccc5c(-c5ccc6oc7cccc(-c8c9ccccc9c(-c9cccc%10c9c9ccccc9n%10-c9ccccc9)c9ccccc89)c7c6c5)c5ccccc45)cccc32)cc1 ; c1coc(-c2c3ccccc3c(-c3ccc4oc5cccc(-c6c7ccccc7c(-c7ccco7)c7ccccc67)c5c4c3)c3ccccc23)c1]

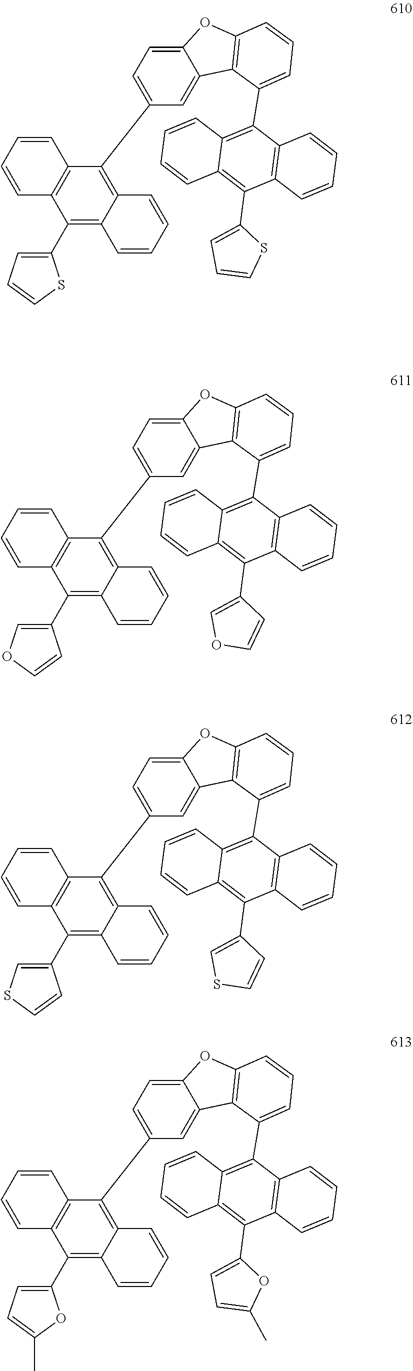 [SMILES: c1csc(-c2c3ccccc3c(-c3ccc4oc5cccc(-c6c7ccccc7c(-c7cccs7)c7ccccc67)c5c4c3)c3ccccc23)c1 ; c1cc(-c2c3ccccc3c(-c3ccoc3)c3ccccc23)c2c(c1)oc1ccc(-c3c4ccccc4c(-c4ccoc4)c4ccccc34)cc12 ; c1cc(-c2c3ccccc3c(-c3ccsc3)c3ccccc23)c2c(c1)oc1ccc(-c3c4ccccc4c(-c4ccsc4)c4ccccc34)cc12 ; Cc1ccc(-c2c3ccccc3c(-c3ccc4oc5cccc(-c6c7ccccc7c(-c7ccc(C)o7)c7ccccc67)c5c4c3)c3ccccc23)o1]

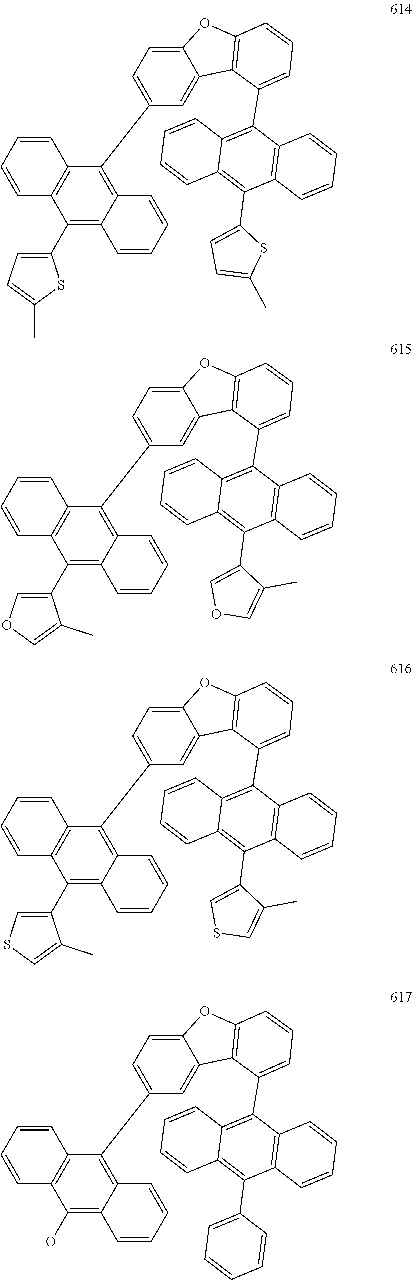 [SMILES: Cc1ccc(-c2c3ccccc3c(-c3ccc4oc5cccc(-c6c7ccccc7c(-c7ccc(C)s7)c7ccccc67)c5c4c3)c3ccccc23)s1 ; Cc1cocc1-c1c2ccccc2c(-c2ccc3oc4cccc(-c5c6ccccc6c(-c6cocc6C)c6ccccc56)c4c3c2)c2ccccc12 ; Cc1cscc1-c1c2ccccc2c(-c2ccc3oc4cccc(-c5c6ccccc6c(-c6cscc6C)c6ccccc56)c4c3c2)c2ccccc12 ; Oc1c2ccccc2c(-c2ccc3oc4cccc(-c5c6ccccc6c(-c6ccccc6)c6ccccc56)c4c3c2)c2ccccc12]

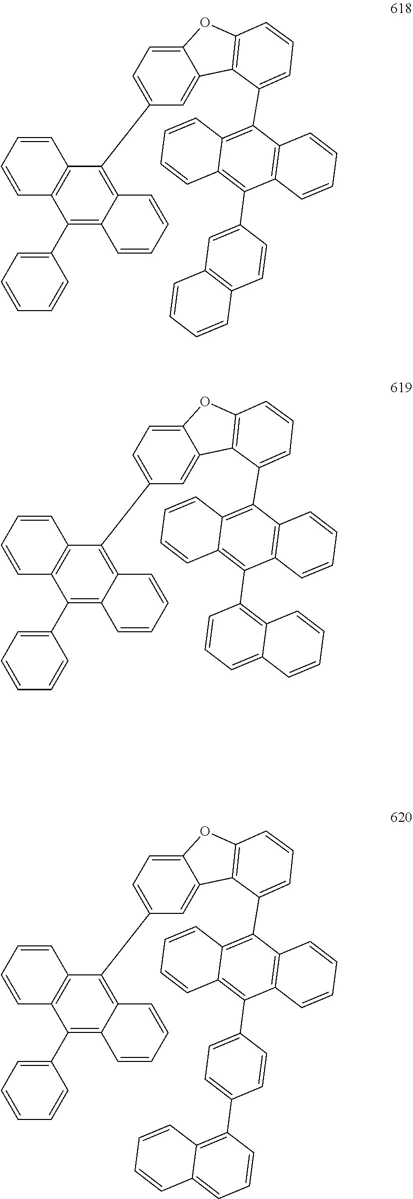 [SMILES: c1ccc(-c2c3ccccc3c(-c3ccc4oc5cccc(-c6c7ccccc7c(-c7ccc8ccccc8c7)c7ccccc67)c5c4c3)c3ccccc23)cc1 ; c1ccc(-c2c3ccccc3c(-c3ccc4oc5cccc(-c6c7ccccc7c(-c7cccc8ccccc78)c7ccccc67)c5c4c3)c3ccccc23)cc1 ; c1ccc(-c2c3ccccc3c(-c3ccc4oc5cccc(-c6c7ccccc7c(-c7ccc(-c8cccc9ccccc89)cc7)c7ccccc67)c5c4c3)c3ccccc23)cc1]

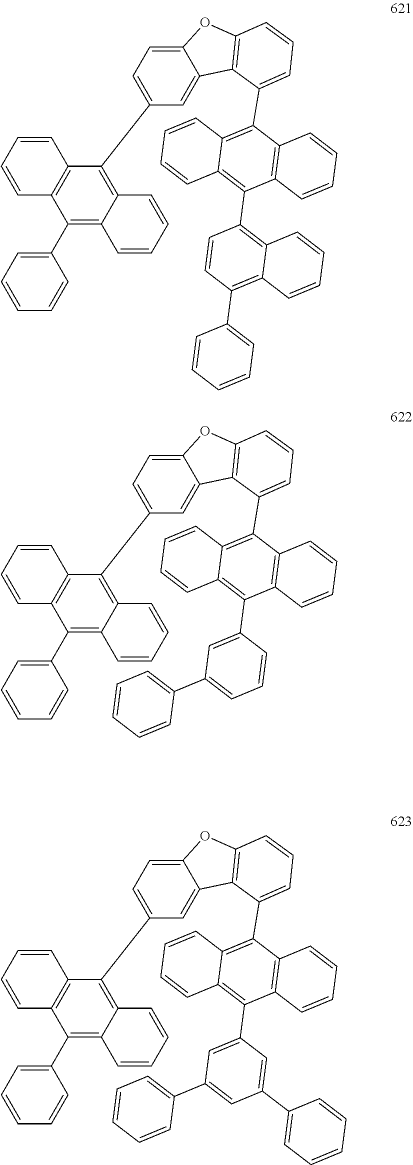

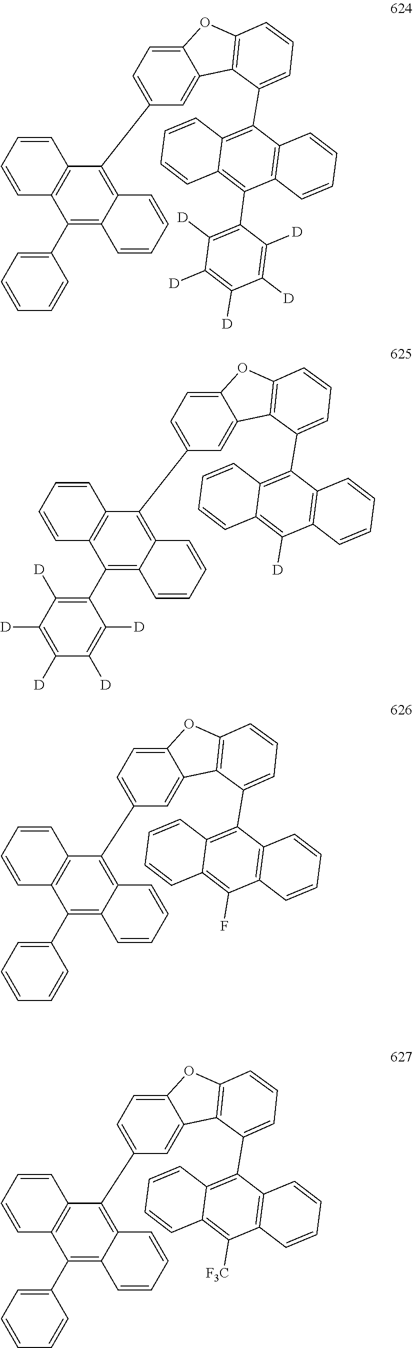 [SMILES: [2H]c1c([2H])c([2H])c(-c2c3ccccc3c(-c3cccc4oc5ccc(-c6c7ccccc7c(-c7ccccc7)c7ccccc67)cc5c34)c3ccccc23)c([2H])c1[2H] ; [2H]c1c([2H])c([2H])c(-c2c3ccccc3c(-c3ccc4oc5cccc(-c6c7ccccc7c([2H])c7ccccc67)c5c4c3)c3ccccc23)c([2H])c1[2H] ; Fc1c2ccccc2c(-c2cccc3oc4ccc(-c5c6ccccc6c(-c6ccccc6)c6ccccc56)cc4c23)c2ccccc12 ; FC(F)(F)c1c2ccccc2c(-c2cccc3oc4ccc(-c5c6ccccc6c(-c6ccccc6)c6ccccc56)cc4c23)c2ccccc12]

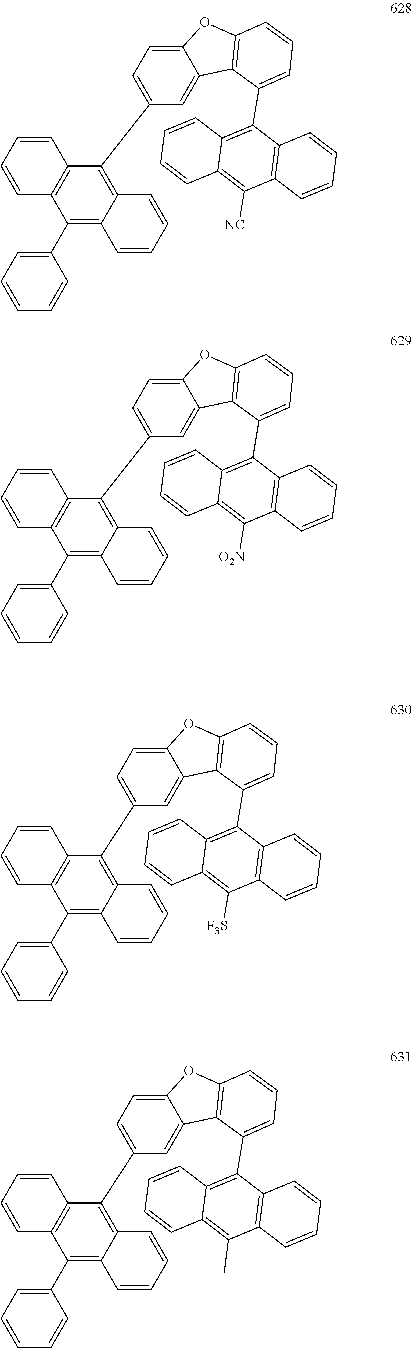

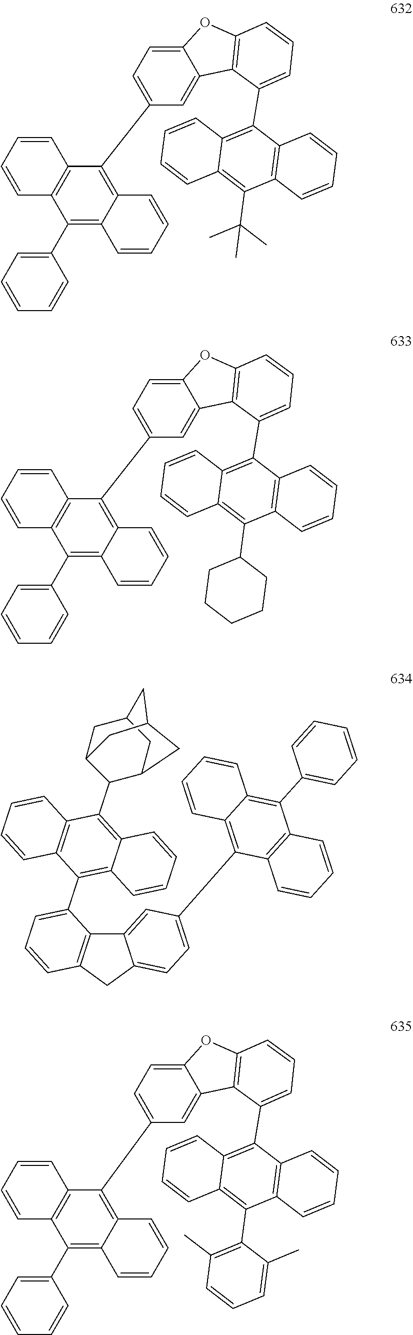

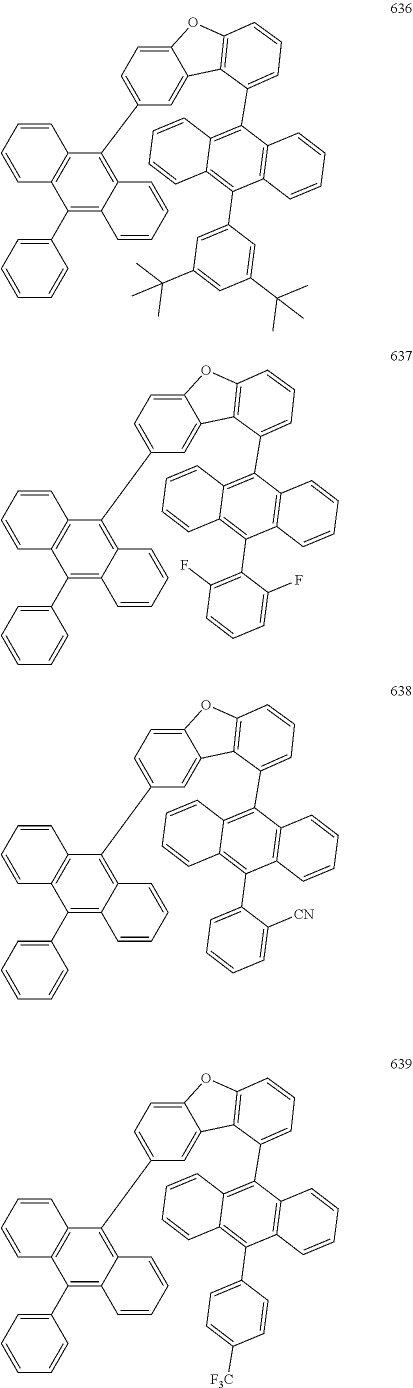

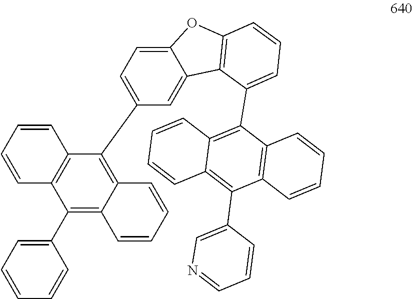 [SMILES: c1ccc(-c2c3ccccc3c(-c3ccc4oc5cccc(-c6c7ccccc7c(-c7cccnc7)c7ccccc67)c5c4c3)c3ccccc23)cc1]

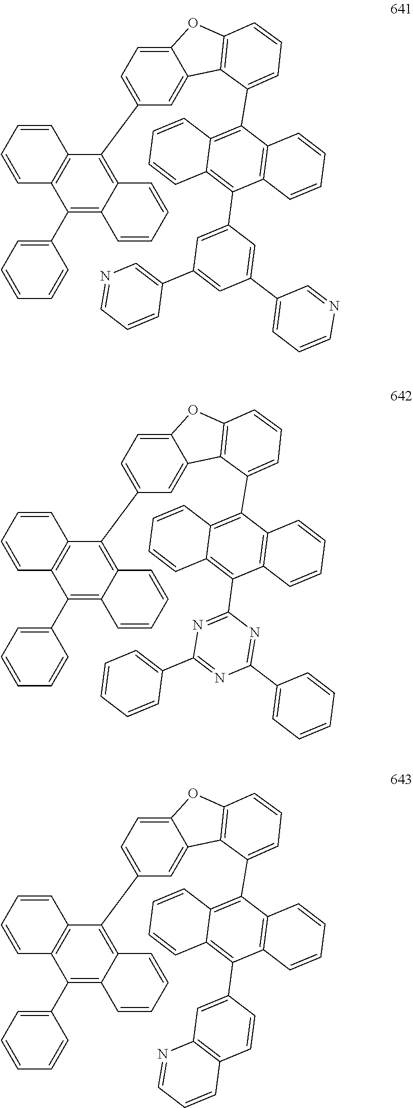 [SMILES: c1ccc(-c2c3ccccc3c(-c3ccc4oc5cccc(-c6c7ccccc7c(-c7cc(-c8cccnc8)cc(-c8cccnc8)c7)c7ccccc67)c5c4c3)c3ccccc23)cc1 ; c1ccc(-c2nc(-c3ccccc3)nc(-c3c4ccccc4c(-c4cccc5oc6ccc(-c7c8ccccc8c(-c8ccccc8)c8ccccc78)cc6c45)c4ccccc34)n2)cc1 ; c1ccc(-c2c3ccccc3c(-c3ccc4oc5cccc(-c6c7ccccc7c(-c7ccc8cccnc8c7)c7ccccc67)c5c4c3)c3ccccc23)cc1]

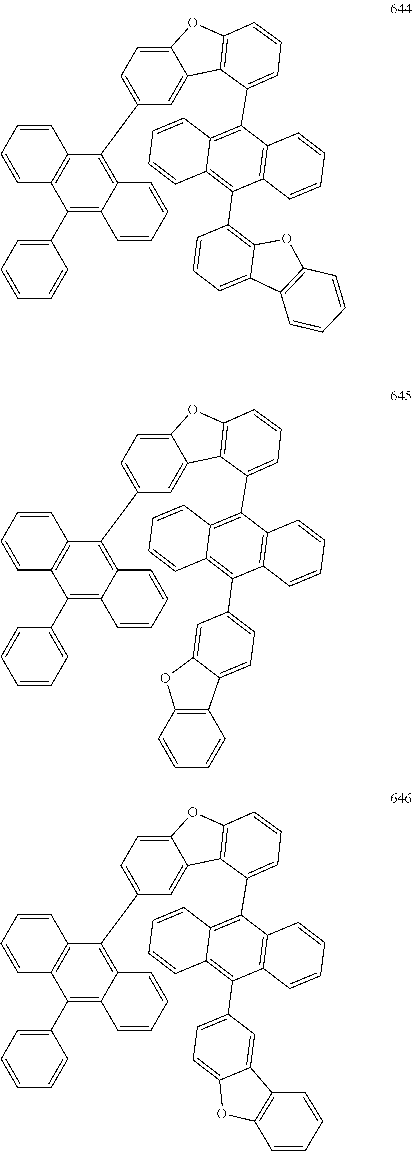 [SMILES: c1ccc(-c2c3ccccc3c(-c3ccc4oc5cccc(-c6c7ccccc7c(-c7cccc8c7oc7ccccc78)c7ccccc67)c5c4c3)c3ccccc23)cc1 ; c1ccc(-c2c3ccccc3c(-c3ccc4oc5cccc(-c6c7ccccc7c(-c7ccc8c(c7)oc7ccccc78)c7ccccc67)c5c4c3)c3ccccc23)cc1 ; c1ccc(-c2c3ccccc3c(-c3ccc4oc5cccc(-c6c7ccccc7c(-c7ccc8oc9ccccc9c8c7)c7ccccc67)c5c4c3)c3ccccc23)cc1]

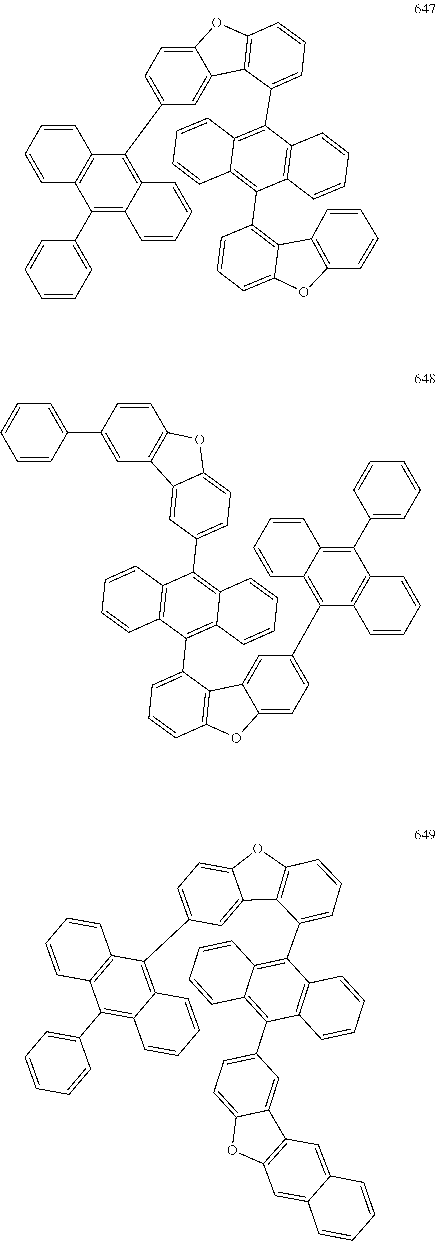 [SMILES: c1ccc(-c2c3ccccc3c(-c3ccc4oc5cccc(-c6c7ccccc7c(-c7cccc8oc9ccccc9c78)c7ccccc67)c5c4c3)c3ccccc23)cc1 ; c1ccc(-c2ccc3oc4ccc(-c5c6ccccc6c(-c6cccc7oc8ccc(-c9c%10ccccc%10c(-c%10ccccc%10)c%10ccccc9%10)cc8c67)c6ccccc56)cc4c3c2)cc1 ; c1ccc(-c2c3ccccc3c(-c3ccc4oc5cccc(-c6c7ccccc7c(-c7ccc8oc9cc%10ccccc%10cc9c8c7)c7ccccc67)c5c4c3)c3ccccc23)cc1]

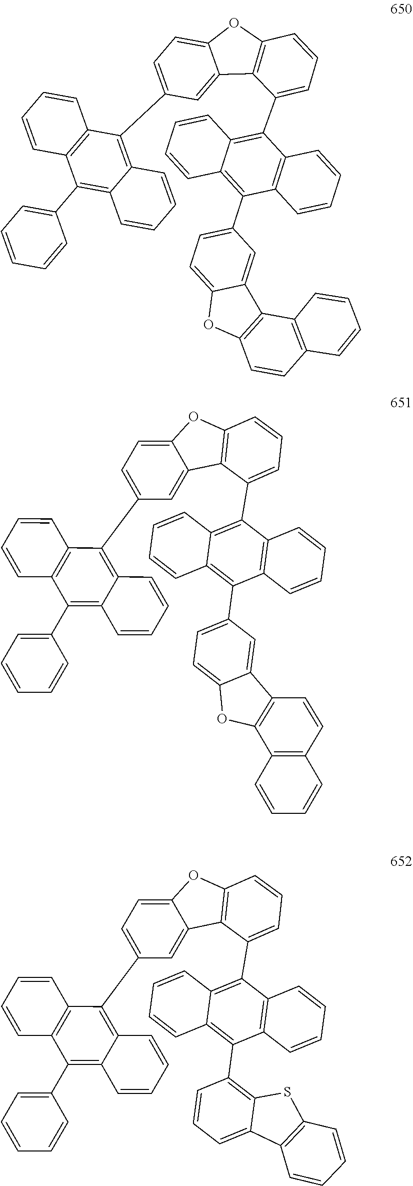 [SMILES: c1ccc(-c2c3ccccc3c(-c3ccc4oc5cccc(-c6c7ccccc7c(-c7ccc8oc9ccc%10ccccc%10c9c8c7)c7ccccc67)c5c4c3)c3ccccc23)cc1 ; c1ccc(-c2c3ccccc3c(-c3ccc4oc5cccc(-c6c7ccccc7c(-c7ccc8oc9c%10ccccc%10ccc9c8c7)c7ccccc67)c5c4c3)c3ccccc23)cc1 ; c1ccc(-c2c3ccccc3c(-c3ccc4oc5cccc(-c6c7ccccc7c(-c7cccc8c7sc7ccccc78)c7ccccc67)c5c4c3)c3ccccc23)cc1]

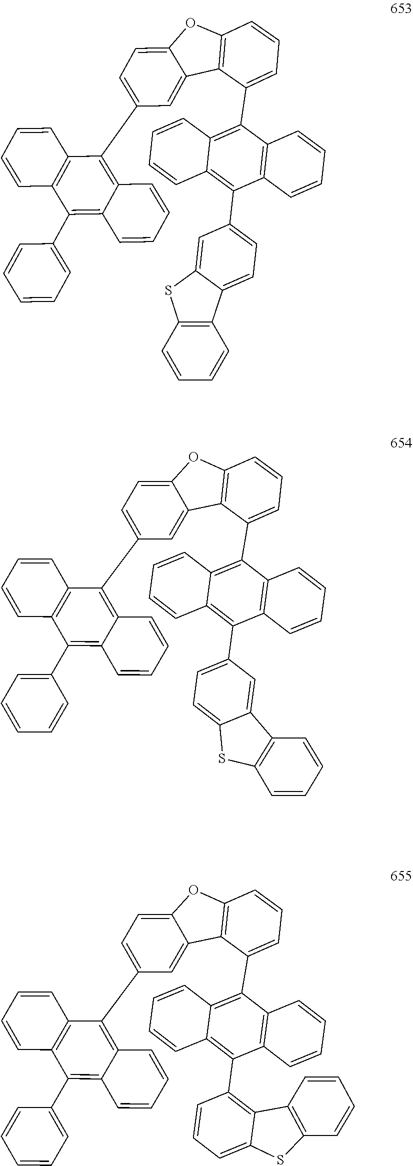 [SMILES: c1ccc(-c2c3ccccc3c(-c3ccc4oc5cccc(-c6c7ccccc7c(-c7ccc8c(c7)sc7ccccc78)c7ccccc67)c5c4c3)c3ccccc23)cc1 ; c1ccc(-c2c3ccccc3c(-c3ccc4oc5cccc(-c6c7ccccc7c(-c7ccc8sc9ccccc9c8c7)c7ccccc67)c5c4c3)c3ccccc23)cc1 ; c1ccc(-c2c3ccccc3c(-c3ccc4oc5cccc(-c6c7ccccc7c(-c7cccc8sc9ccccc9c78)c7ccccc67)c5c4c3)c3ccccc23)cc1]

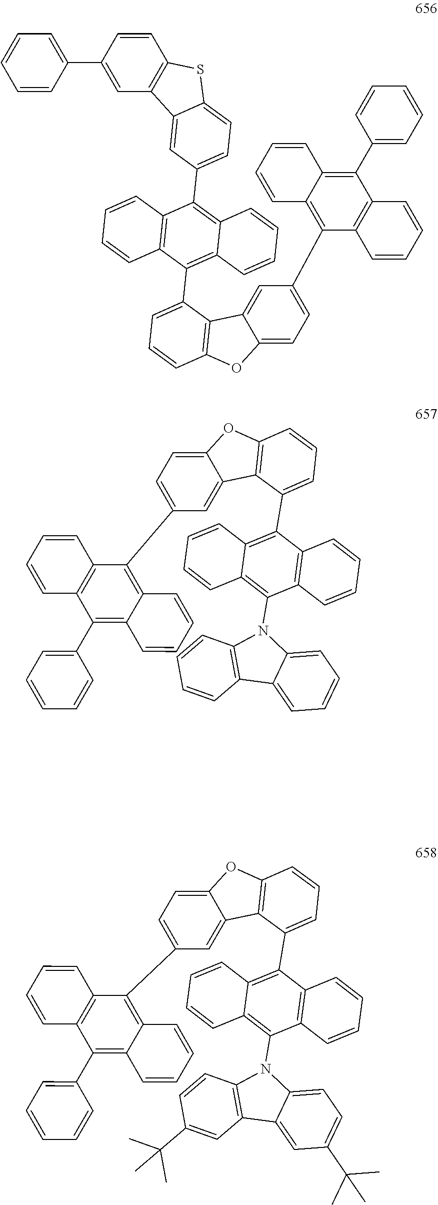 [SMILES: c1ccc(-c2ccc3sc4ccc(-c5c6ccccc6c(-c6cccc7oc8ccc(-c9c%10ccccc%10c(-c%10ccccc%10)c%10ccccc9%10)cc8c67)c6ccccc56)cc4c3c2)cc1 ; c1ccc(-c2c3ccccc3c(-c3ccc4oc5cccc(-c6c7ccccc7c(-n7c8ccccc8c8ccccc87)c7ccccc67)c5c4c3)c3ccccc23)cc1 ; CC(C)(C)c1ccc2c(c1)c1cc(C(C)(C)C)ccc1n2-c1c2ccccc2c(-c2cccc3oc4ccc(-c5c6ccccc6c(-c6ccccc6)c6ccccc56)cc4c23)c2ccccc12]

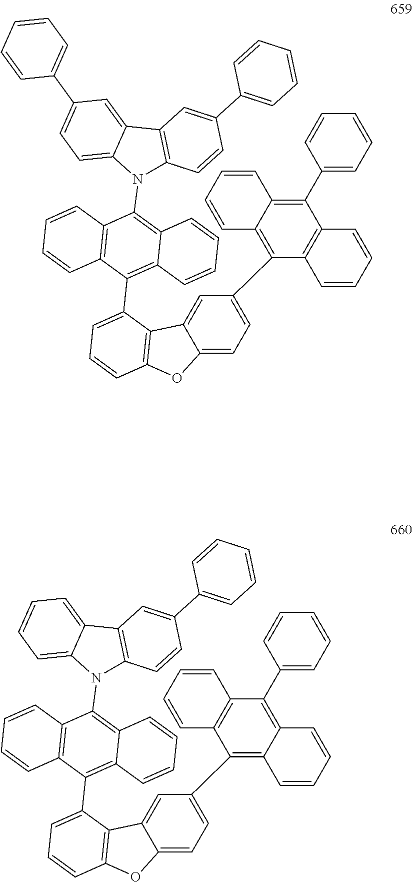 [SMILES: c1ccc(-c2ccc3c(c2)c2cc(-c4ccccc4)ccc2n3-c2c3ccccc3c(-c3cccc4oc5ccc(-c6c7ccccc7c(-c7ccccc7)c7ccccc67)cc5c34)c3ccccc23)cc1 ; c1ccc(-c2ccc3c(c2)c2ccccc2n3-c2c3ccccc3c(-c3cccc4oc5ccc(-c6c7ccccc7c(-c7ccccc7)c7ccccc67)cc5c34)c3ccccc23)cc1]

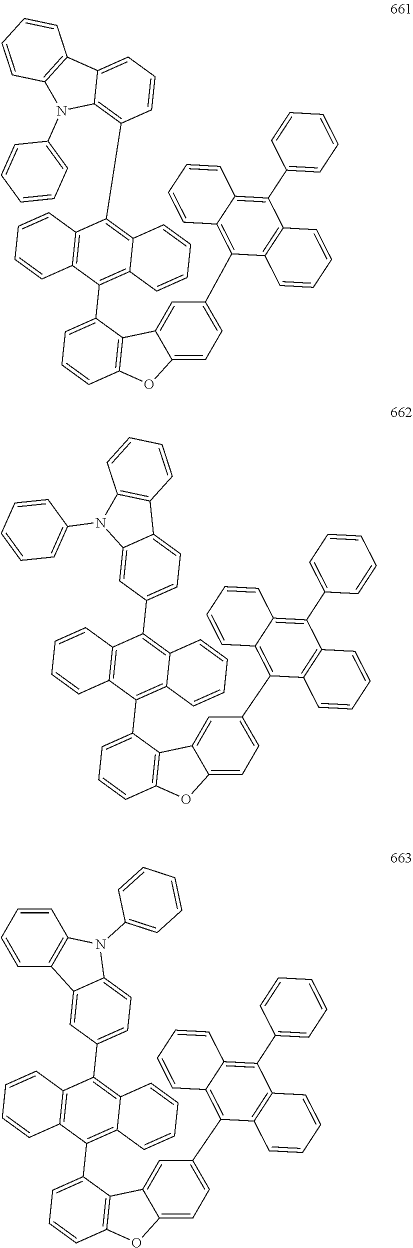 [SMILES: c1ccc(-c2c3ccccc3c(-c3ccc4oc5cccc(-c6c7ccccc7c(-c7cccc8c9ccccc9n(-c9ccccc9)c78)c7ccccc67)c5c4c3)c3ccccc23)cc1 ; c1ccc(-c2c3ccccc3c(-c3ccc4oc5cccc(-c6c7ccccc7c(-c7ccc8c9ccccc9n(-c9ccccc9)c8c7)c7ccccc67)c5c4c3)c3ccccc23)cc1 ; c1ccc(-c2c3ccccc3c(-c3ccc4oc5cccc(-c6c7ccccc7c(-c7ccc8c(c7)c7ccccc7n8-c7ccccc7)c7ccccc67)c5c4c3)c3ccccc23)cc1]

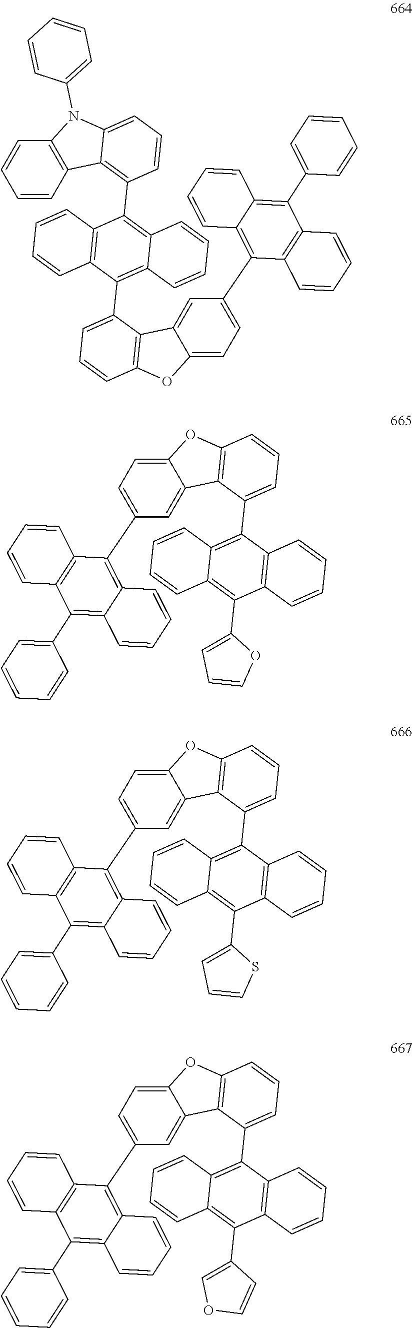 [SMILES: c1ccc(-c2c3ccccc3c(-c3ccc4oc5cccc(-c6c7ccccc7c(-c7cccc8c7c7ccccc7n8-c7ccccc7)c7ccccc67)c5c4c3)c3ccccc23)cc1 ; c1ccc(-c2c3ccccc3c(-c3ccc4oc5cccc(-c6c7ccccc7c(-c7ccco7)c7ccccc67)c5c4c3)c3ccccc23)cc1 ; c1ccc(-c2c3ccccc3c(-c3ccc4oc5cccc(-c6c7ccccc7c(-c7cccs7)c7ccccc67)c5c4c3)c3ccccc23)cc1 ; c1ccc(-c2c3ccccc3c(-c3ccc4oc5cccc(-c6c7ccccc7c(-c7ccoc7)c7ccccc67)c5c4c3)c3ccccc23)cc1]

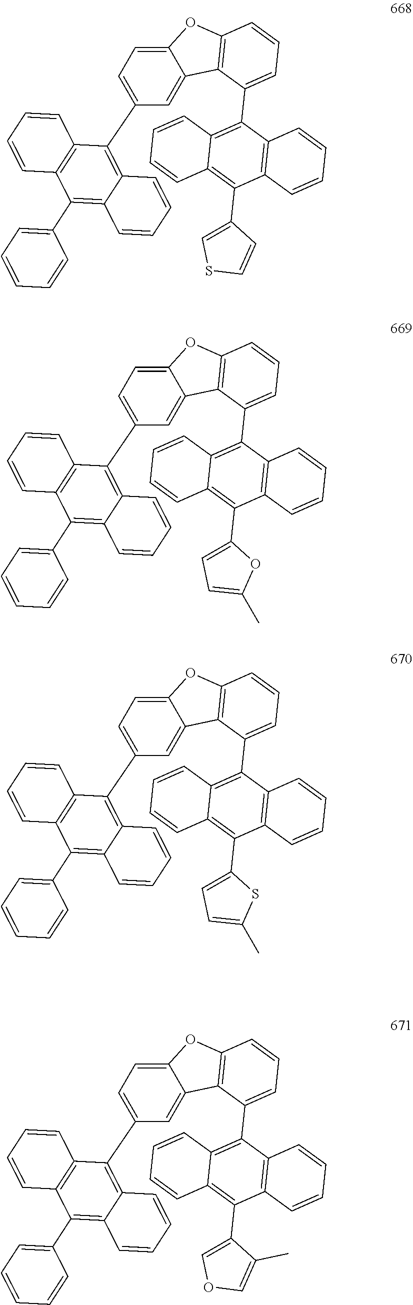 [SMILES: c1ccc(-c2c3ccccc3c(-c3ccc4oc5cccc(-c6c7ccccc7c(-c7ccsc7)c7ccccc67)c5c4c3)c3ccccc23)cc1 ; Cc1ccc(-c2c3ccccc3c(-c3cccc4oc5ccc(-c6c7ccccc7c(-c7ccccc7)c7ccccc67)cc5c34)c3ccccc23)o1 ; Cc1ccc(-c2c3ccccc3c(-c3cccc4oc5ccc(-c6c7ccccc7c(-c7ccccc7)c7ccccc67)cc5c34)c3ccccc23)s1 ; Cc1cocc1-c1c2ccccc2c(-c2cccc3oc4ccc(-c5c6ccccc6c(-c6ccccc6)c6ccccc56)cc4c23)c2ccccc12]

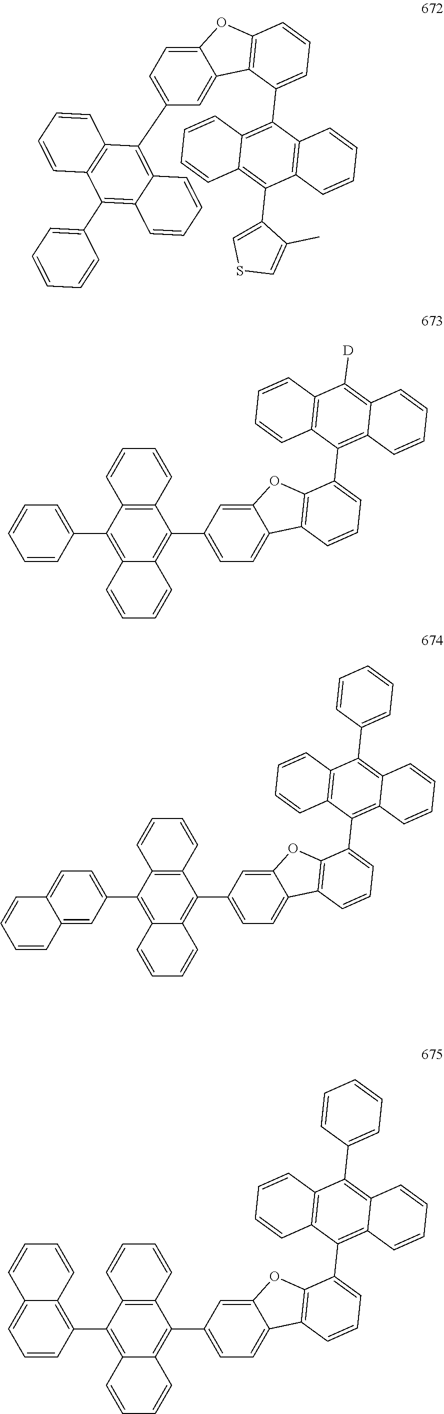 [SMILES: Cc1cscc1-c1c2ccccc2c(-c2cccc3oc4ccc(-c5c6ccccc6c(-c6ccccc6)c6ccccc56)cc4c23)c2ccccc12 ; [2H]c1c2ccccc2c(-c2cccc3c2oc2cc(-c4c5ccccc5c(-c5ccccc5)c5ccccc45)ccc23)c2ccccc12 ; c1ccc(-c2c3ccccc3c(-c3cccc4c3oc3cc(-c5c6ccccc6c(-c6ccc7ccccc7c6)c6ccccc56)ccc34)c3ccccc23)cc1 ; c1ccc(-c2c3ccccc3c(-c3cccc4c3oc3cc(-c5c6ccccc6c(-c6cccc7ccccc67)c6ccccc56)ccc34)c3ccccc23)cc1]

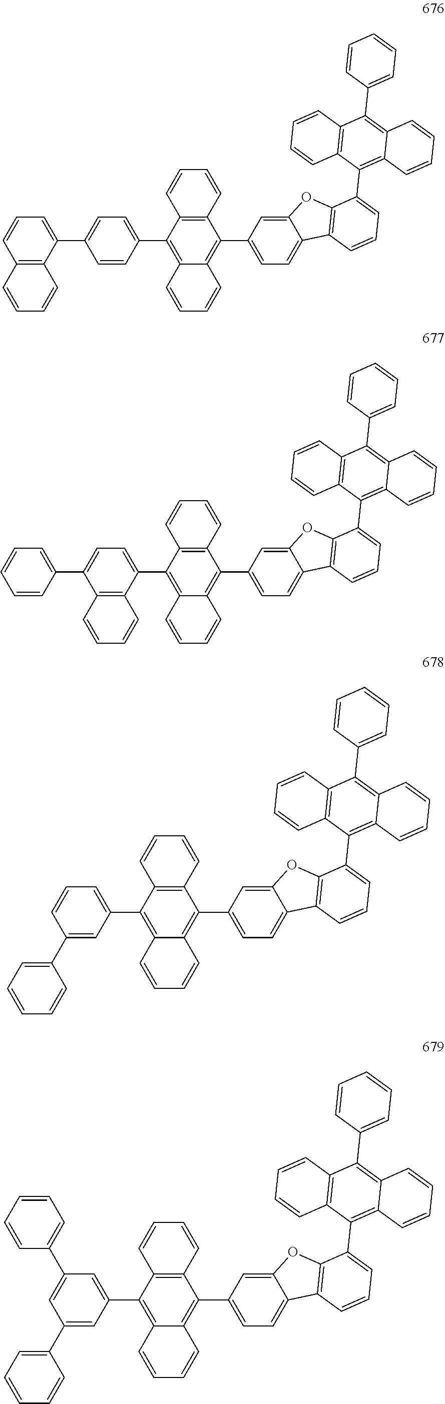 [SMILES: c1ccc(-c2c3ccccc3c(-c3cccc4c3oc3cc(-c5c6ccccc6c(-c6ccc(-c7cccc8ccccc78)cc6)c6ccccc56)ccc34)c3ccccc23)cc1 ; c1ccc(-c2ccc(-c3c4ccccc4c(-c4ccc5c(c4)oc4c(-c6c7ccccc7c(-c7ccccc7)c7ccccc67)cccc45)c4ccccc34)c3ccccc23)cc1 ; c1ccc(-c2cccc(-c3c4ccccc4c(-c4ccc5c(c4)oc4c(-c6c7ccccc7c(-c7ccccc7)c7ccccc67)cccc45)c4ccccc34)c2)cc1 ; c1ccc(-c2cc(-c3ccccc3)cc(-c3c4ccccc4c(-c4ccc5c(c4)oc4c(-c6c7ccccc7c(-c7ccccc7)c7ccccc67)cccc45)c4ccccc34)c2)cc1]

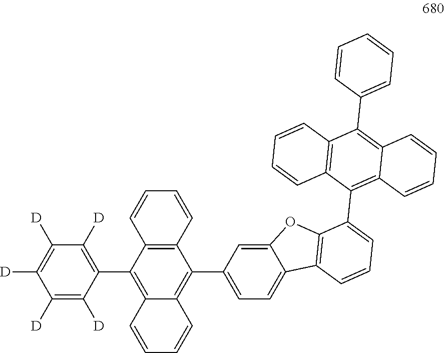 [SMILES: [2H]c1c([2H])c([2H])c(-c2c3ccccc3c(-c3ccc4c(c3)oc3c(-c5c6ccccc6c(-c6ccccc6)c6ccccc56)cccc34)c3ccccc23)c([2H])c1[2H]]

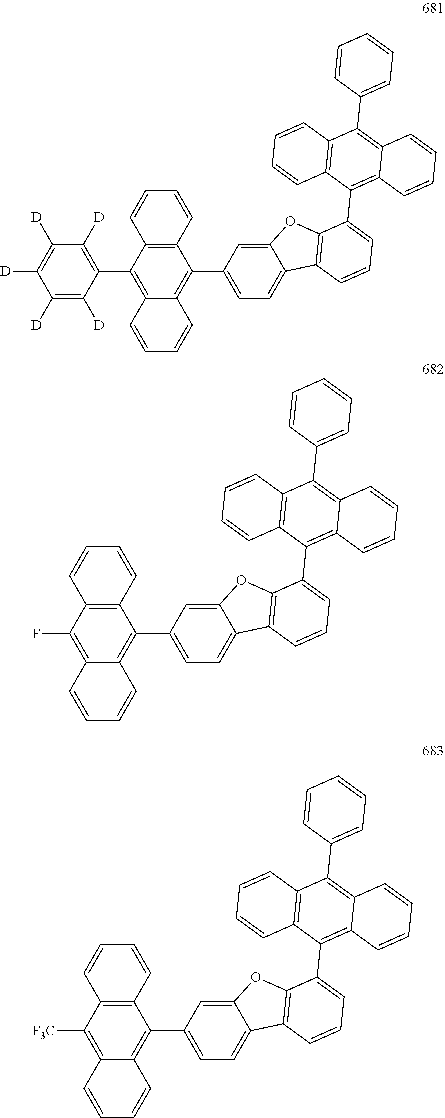 [SMILES: [2H]c1c([2H])c([2H])c(-c2c3ccccc3c(-c3ccc4c(c3)oc3c(-c5c6ccccc6c(-c6ccccc6)c6ccccc56)cccc34)c3ccccc23)c([2H])c1[2H] ; Fc1c2ccccc2c(-c2ccc3c(c2)oc2c(-c4c5ccccc5c(-c5ccccc5)c5ccccc45)cccc23)c2ccccc12 ; FC(F)(F)c1c2ccccc2c(-c2ccc3c(c2)oc2c(-c4c5ccccc5c(-c5ccccc5)c5ccccc45)cccc23)c2ccccc12]

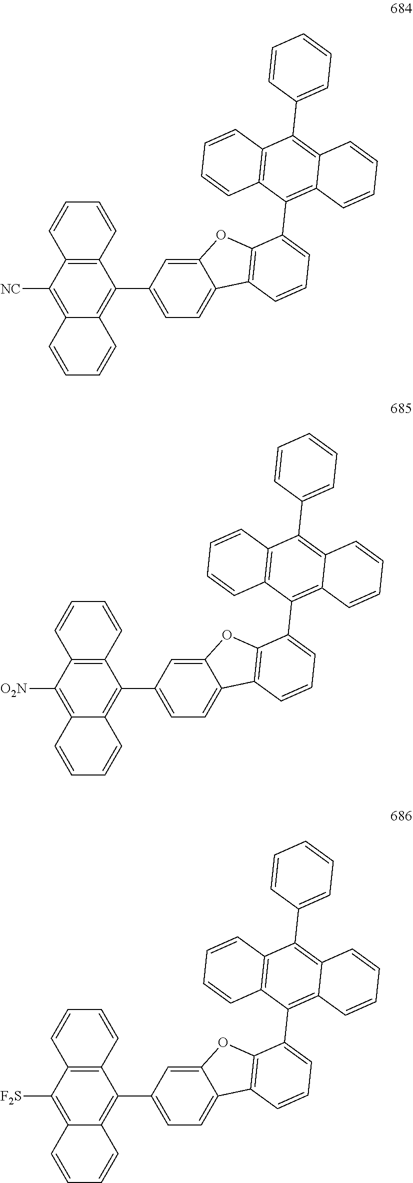

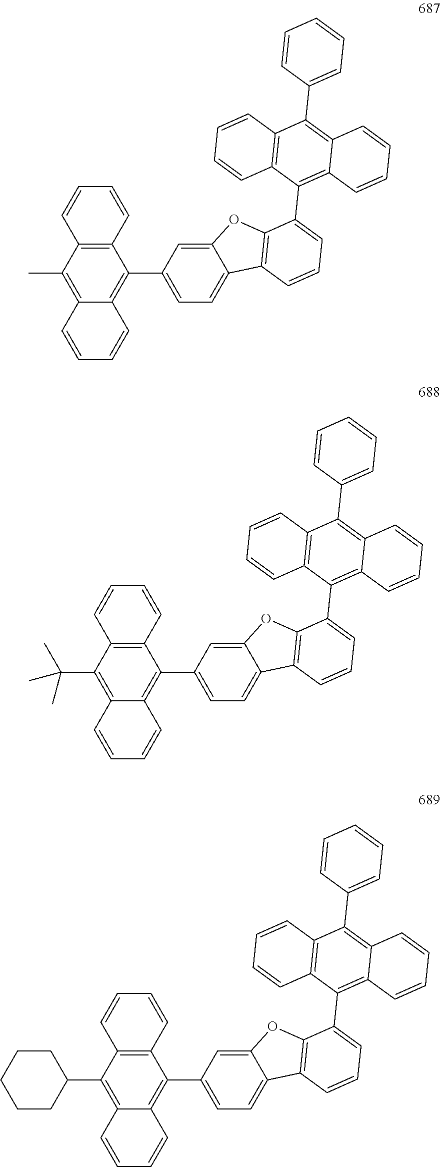 [SMILES: Cc1c2ccccc2c(-c2ccc3c(c2)oc2c(-c4c5ccccc5c(-c5ccccc5)c5ccccc45)cccc23)c2ccccc12 ; CC(C)(C)c1c2ccccc2c(-c2ccc3c(c2)oc2c(-c4c5ccccc5c(-c5ccccc5)c5ccccc45)cccc23)c2ccccc12 ; c1ccc(-c2c3ccccc3c(-c3cccc4c3oc3cc(-c5c6ccccc6c(C6CCCCC6)c6ccccc56)ccc34)c3ccccc23)cc1]

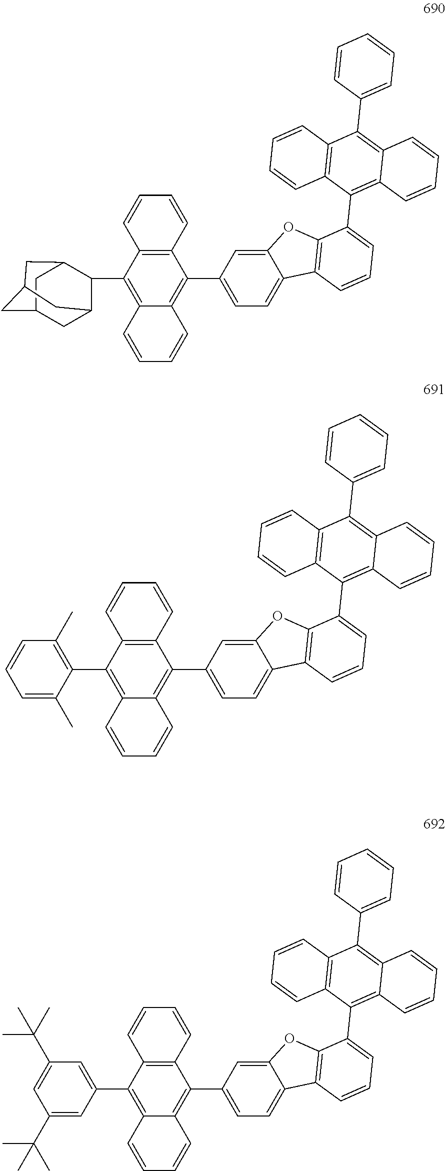 [SMILES: c1ccc(-c2c3ccccc3c(-c3cccc4c3oc3cc(-c5c6ccccc6c(C6C7CC8CC(C7)CC6C8)c6ccccc56)ccc34)c3ccccc23)cc1 ; Cc1cccc(C)c1-c1c2ccccc2c(-c2ccc3c(c2)oc2c(-c4c5ccccc5c(-c5ccccc5)c5ccccc45)cccc23)c2ccccc12 ; CC(C)(C)c1cc(-c2c3ccccc3c(-c3ccc4c(c3)oc3c(-c5c6ccccc6c(-c6ccccc6)c6ccccc56)cccc34)c3ccccc23)cc(C(C)(C)C)c1]

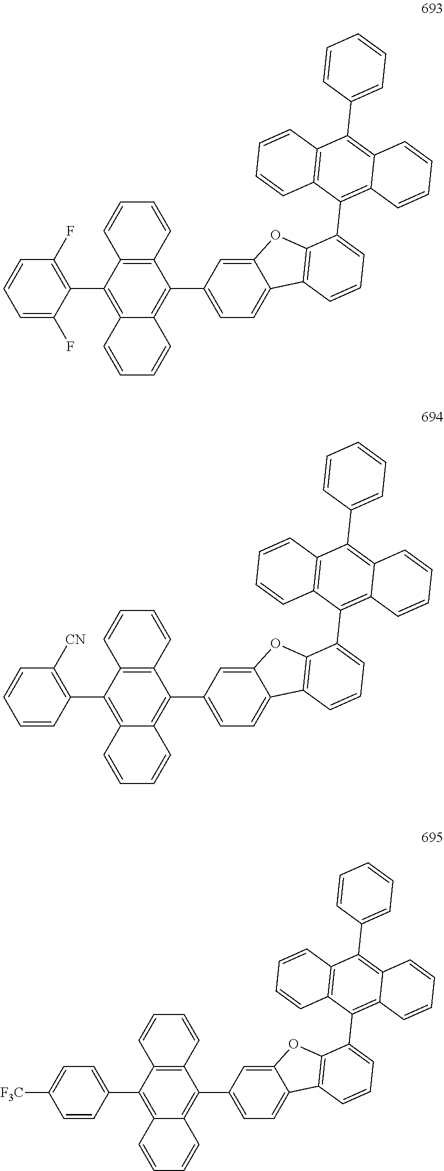 [SMILES: Fc1cccc(F)c1-c1c2ccccc2c(-c2ccc3c(c2)oc2c(-c4c5ccccc5c(-c5ccccc5)c5ccccc45)cccc23)c2ccccc12 ; N#Cc1ccccc1-c1c2ccccc2c(-c2ccc3c(c2)oc2c(-c4c5ccccc5c(-c5ccccc5)c5ccccc45)cccc23)c2ccccc12 ; FC(F)(F)c1ccc(-c2c3ccccc3c(-c3ccc4c(c3)oc3c(-c5c6ccccc6c(-c6ccccc6)c6ccccc56)cccc34)c3ccccc23)cc1]

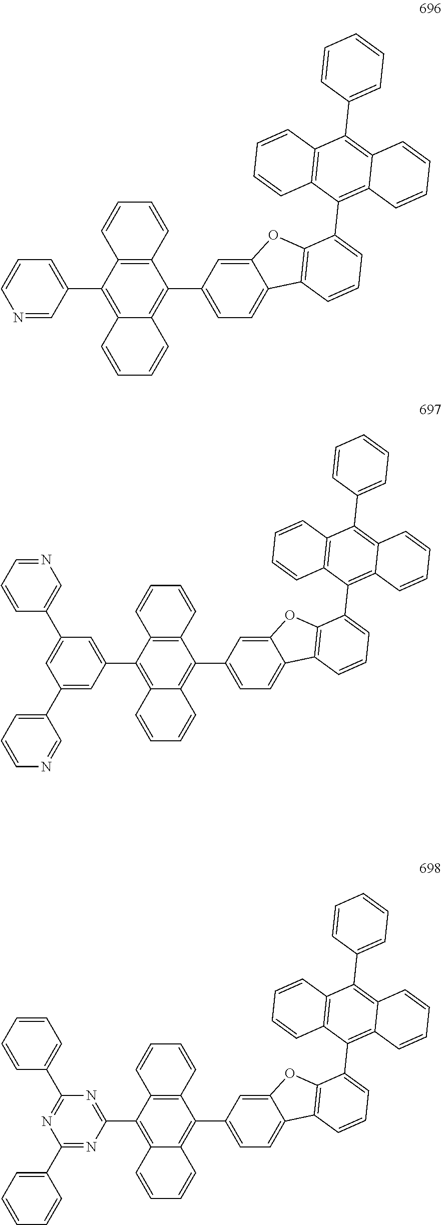 [SMILES: c1ccc(-c2c3ccccc3c(-c3cccc4c3oc3cc(-c5c6ccccc6c(-c6cccnc6)c6ccccc56)ccc34)c3ccccc23)cc1 ; c1ccc(-c2c3ccccc3c(-c3cccc4c3oc3cc(-c5c6ccccc6c(-c6cc(-c7cccnc7)cc(-c7cccnc7)c6)c6ccccc56)ccc34)c3ccccc23)cc1 ; c1ccc(-c2nc(-c3ccccc3)nc(-c3c4ccccc4c(-c4ccc5c(c4)oc4c(-c6c7ccccc7c(-c7ccccc7)c7ccccc67)cccc45)c4ccccc34)n2)cc1]

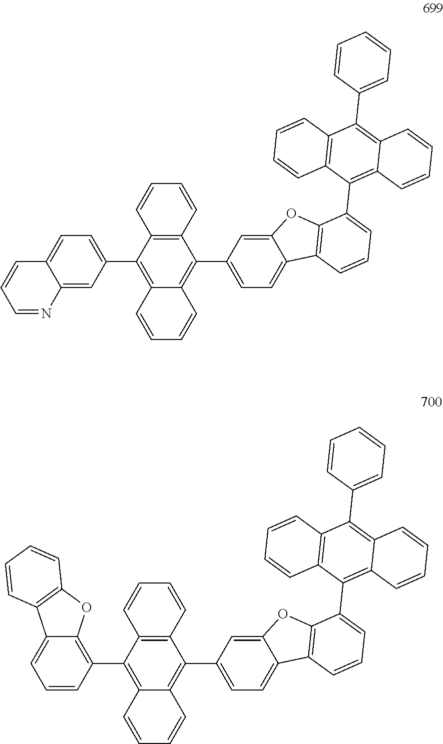 [SMILES: c1ccc(-c2c3ccccc3c(-c3cccc4c3oc3cc(-c5c6ccccc6c(-c6ccc7cccnc7c6)c6ccccc56)ccc34)c3ccccc23)cc1 ; c1ccc(-c2c3ccccc3c(-c3cccc4c3oc3cc(-c5c6ccccc6c(-c6cccc7c6oc6ccccc67)c6ccccc56)ccc34)c3ccccc23)cc1]

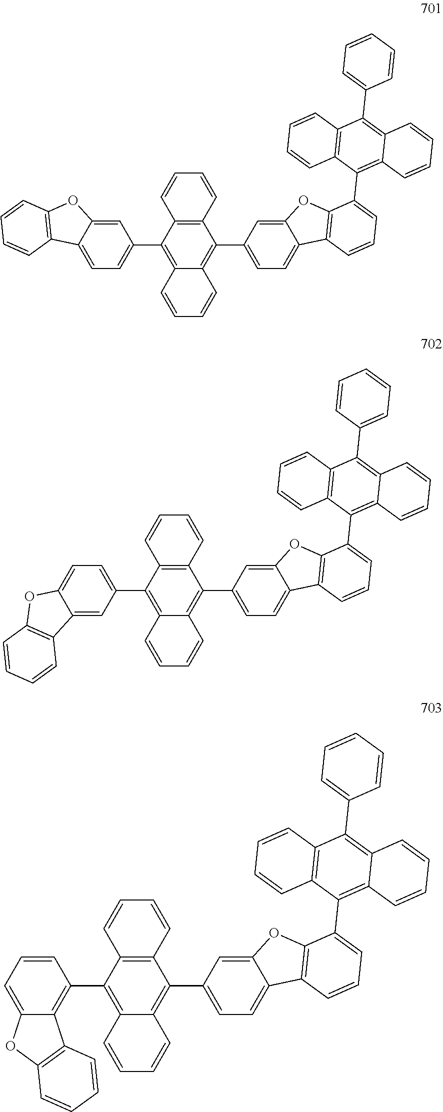 [SMILES: c1ccc(-c2c3ccccc3c(-c3cccc4c3oc3cc(-c5c6ccccc6c(-c6ccc7c(c6)oc6ccccc67)c6ccccc56)ccc34)c3ccccc23)cc1 ; c1ccc(-c2c3ccccc3c(-c3cccc4c3oc3cc(-c5c6ccccc6c(-c6ccc7oc8ccccc8c7c6)c6ccccc56)ccc34)c3ccccc23)cc1 ; c1ccc(-c2c3ccccc3c(-c3cccc4c3oc3cc(-c5c6ccccc6c(-c6cccc7oc8ccccc8c67)c6ccccc56)ccc34)c3ccccc23)cc1]

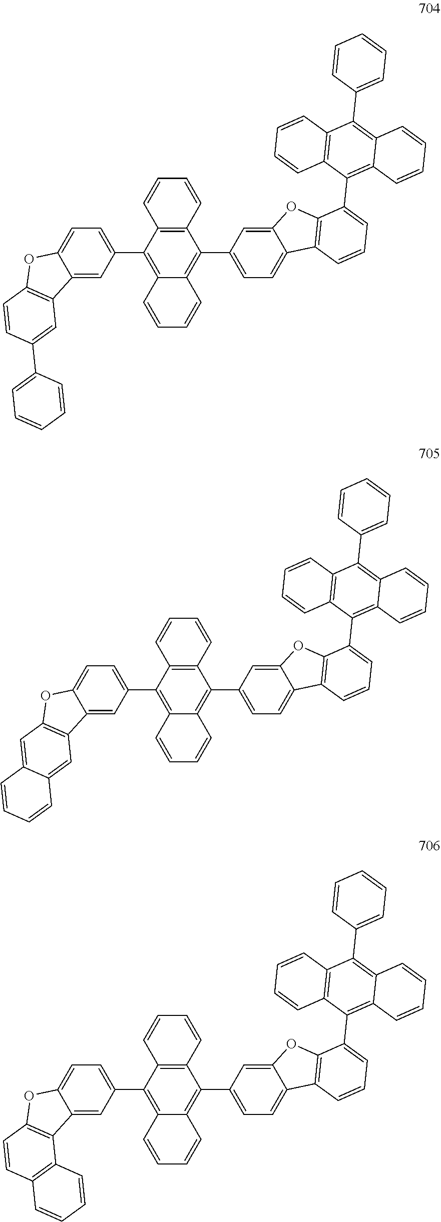 [SMILES: c1ccc(-c2ccc3oc4ccc(-c5c6ccccc6c(-c6ccc7c(c6)oc6c(-c8c9ccccc9c(-c9ccccc9)c9ccccc89)cccc67)c6ccccc56)cc4c3c2)cc1 ; c1ccc(-c2c3ccccc3c(-c3cccc4c3oc3cc(-c5c6ccccc6c(-c6ccc7oc8cc9ccccc9cc8c7c6)c6ccccc56)ccc34)c3ccccc23)cc1 ; c1ccc(-c2c3ccccc3c(-c3cccc4c3oc3cc(-c5c6ccccc6c(-c6ccc7oc8ccc9ccccc9c8c7c6)c6ccccc56)ccc34)c3ccccc23)cc1]

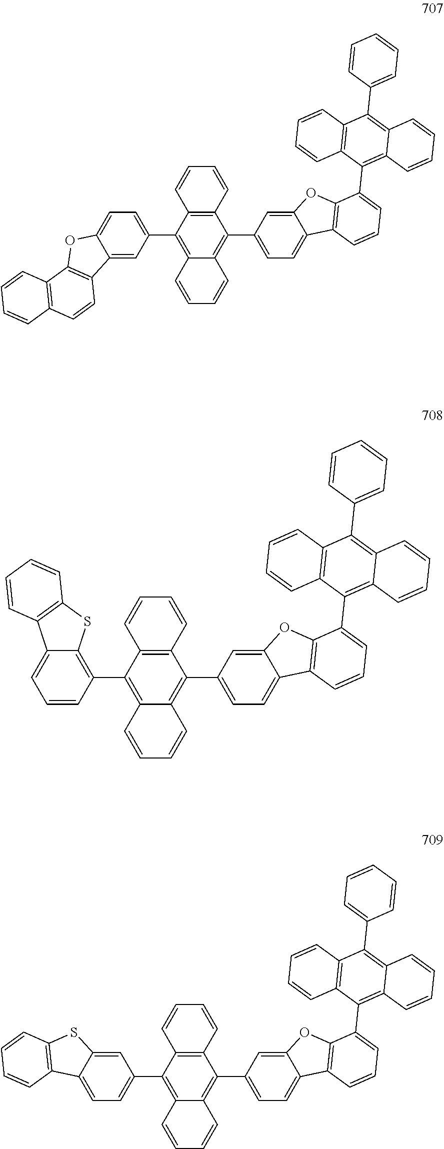 [SMILES: c1ccc(-c2c3ccccc3c(-c3cccc4c3oc3cc(-c5c6ccccc6c(-c6ccc7oc8c9ccccc9ccc8c7c6)c6ccccc56)ccc34)c3ccccc23)cc1 ; c1ccc(-c2c3ccccc3c(-c3cccc4c3oc3cc(-c5c6ccccc6c(-c6cccc7c6sc6ccccc67)c6ccccc56)ccc34)c3ccccc23)cc1 ; c1ccc(-c2c3ccccc3c(-c3cccc4c3oc3cc(-c5c6ccccc6c(-c6ccc7c(c6)sc6ccccc67)c6ccccc56)ccc34)c3ccccc23)cc1]

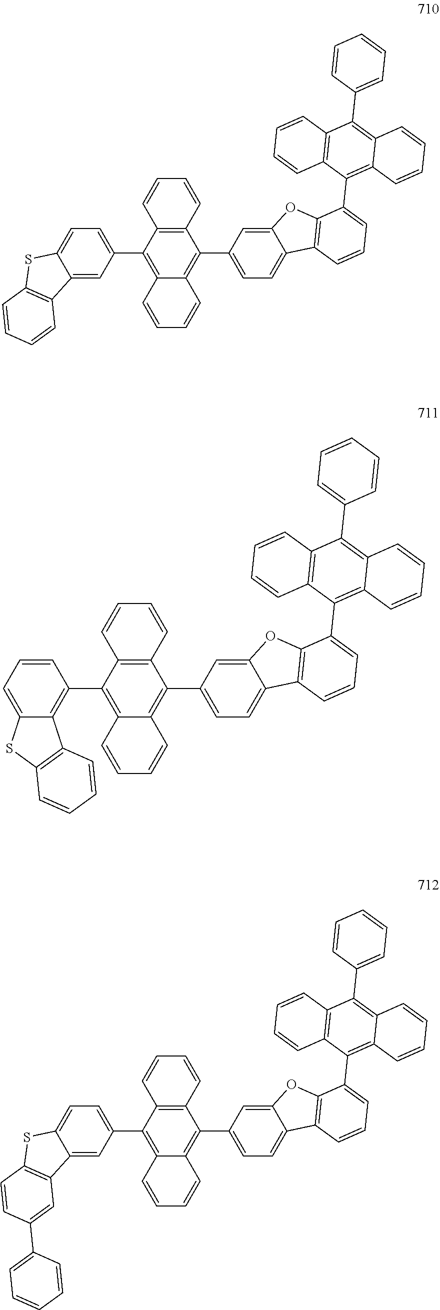 [SMILES: c1ccc(-c2c3ccccc3c(-c3cccc4c3oc3cc(-c5c6ccccc6c(-c6ccc7sc8ccccc8c7c6)c6ccccc56)ccc34)c3ccccc23)cc1 ; c1ccc(-c2c3ccccc3c(-c3cccc4c3oc3cc(-c5c6ccccc6c(-c6cccc7sc8ccccc8c67)c6ccccc56)ccc34)c3ccccc23)cc1 ; c1ccc(-c2ccc3sc4ccc(-c5c6ccccc6c(-c6ccc7c(c6)oc6c(-c8c9ccccc9c(-c9ccccc9)c9ccccc89)cccc67)c6ccccc56)cc4c3c2)cc1]

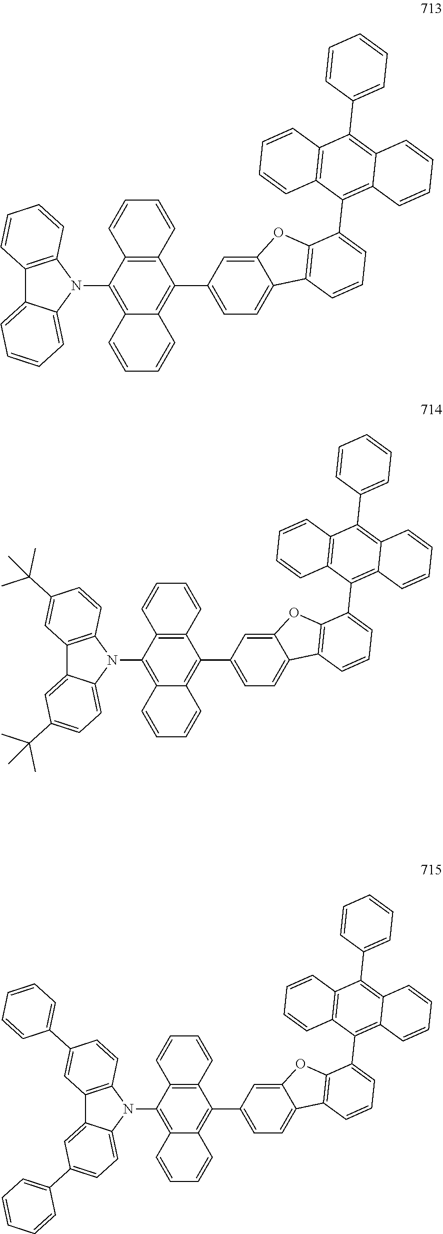 [SMILES: c1ccc(-c2c3ccccc3c(-c3cccc4c3oc3cc(-c5c6ccccc6c(-n6c7ccccc7c7ccccc76)c6ccccc56)ccc34)c3ccccc23)cc1 ; CC(C)(C)c1ccc2c(c1)c1cc(C(C)(C)C)ccc1n2-c1c2ccccc2c(-c2ccc3c(c2)oc2c(-c4c5ccccc5c(-c5ccccc5)c5ccccc45)cccc23)c2ccccc12 ; c1ccc(-c2ccc3c(c2)c2cc(-c4ccccc4)ccc2n3-c2c3ccccc3c(-c3ccc4c(c3)oc3c(-c5c6ccccc6c(-c6ccccc6)c6ccccc56)cccc34)c3ccccc23)cc1]

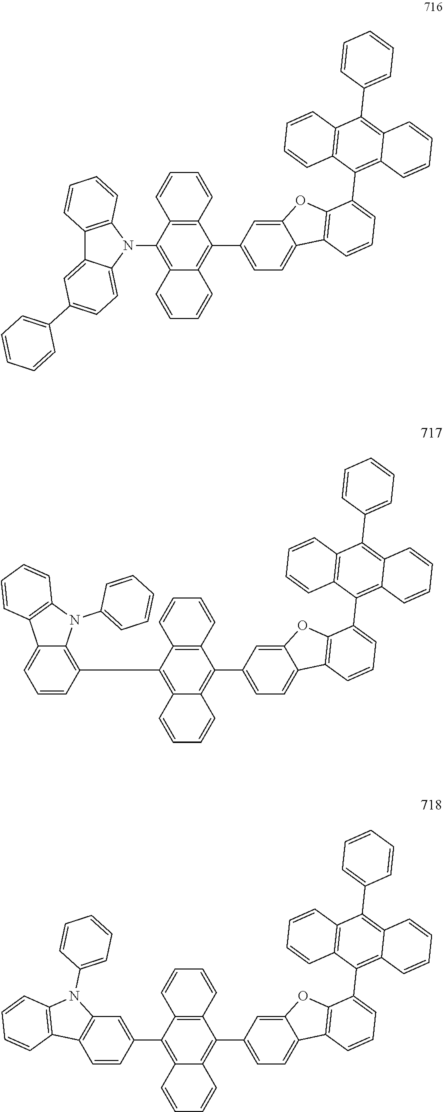 [SMILES: c1ccc(-c2ccc3c(c2)c2ccccc2n3-c2c3ccccc3c(-c3ccc4c(c3)oc3c(-c5c6ccccc6c(-c6ccccc6)c6ccccc56)cccc34)c3ccccc23)cc1 ; c1ccc(-c2c3ccccc3c(-c3cccc4c3oc3cc(-c5c6ccccc6c(-c6cccc7c8ccccc8n(-c8ccccc8)c67)c6ccccc56)ccc34)c3ccccc23)cc1 ; c1ccc(-c2c3ccccc3c(-c3cccc4c3oc3cc(-c5c6ccccc6c(-c6ccc7c8ccccc8n(-c8ccccc8)c7c6)c6ccccc56)ccc34)c3ccccc23)cc1]

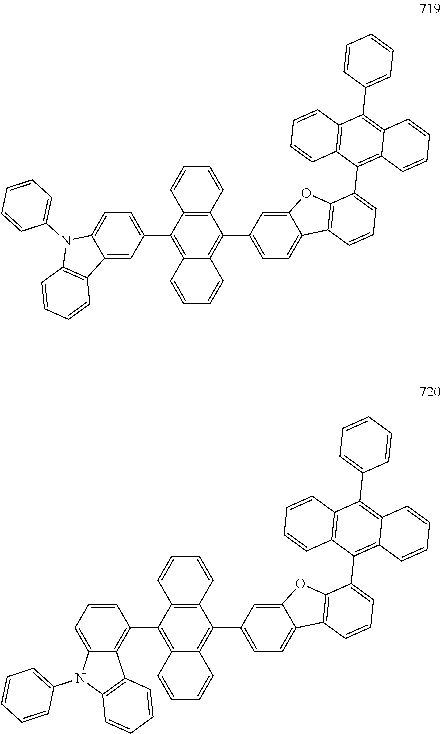 [SMILES: c1ccc(-c2c3ccccc3c(-c3cccc4c3oc3cc(-c5c6ccccc6c(-c6ccc7c(c6)c6ccccc6n7-c6ccccc6)c6ccccc56)ccc34)c3ccccc23)cc1 ; c1ccc(-c2c3ccccc3c(-c3cccc4c3oc3cc(-c5c6ccccc6c(-c6cccc7c6c6ccccc6n7-c6ccccc6)c6ccccc56)ccc34)c3ccccc23)cc1]

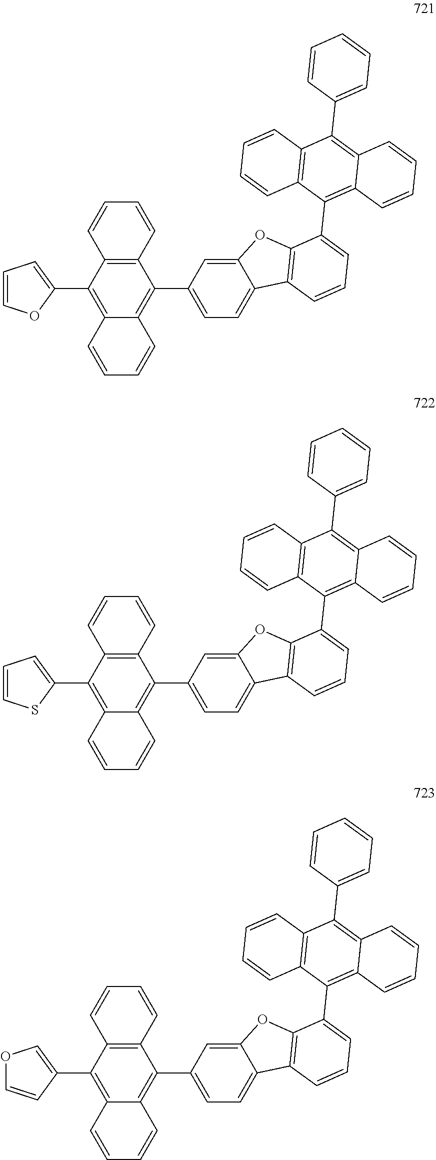 [SMILES: c1ccc(-c2c3ccccc3c(-c3cccc4c3oc3cc(-c5c6ccccc6c(-c6ccco6)c6ccccc56)ccc34)c3ccccc23)cc1 ; c1ccc(-c2c3ccccc3c(-c3cccc4c3oc3cc(-c5c6ccccc6c(-c6cccs6)c6ccccc56)ccc34)c3ccccc23)cc1 ; c1ccc(-c2c3ccccc3c(-c3cccc4c3oc3cc(-c5c6ccccc6c(-c6ccoc6)c6ccccc56)ccc34)c3ccccc23)cc1]

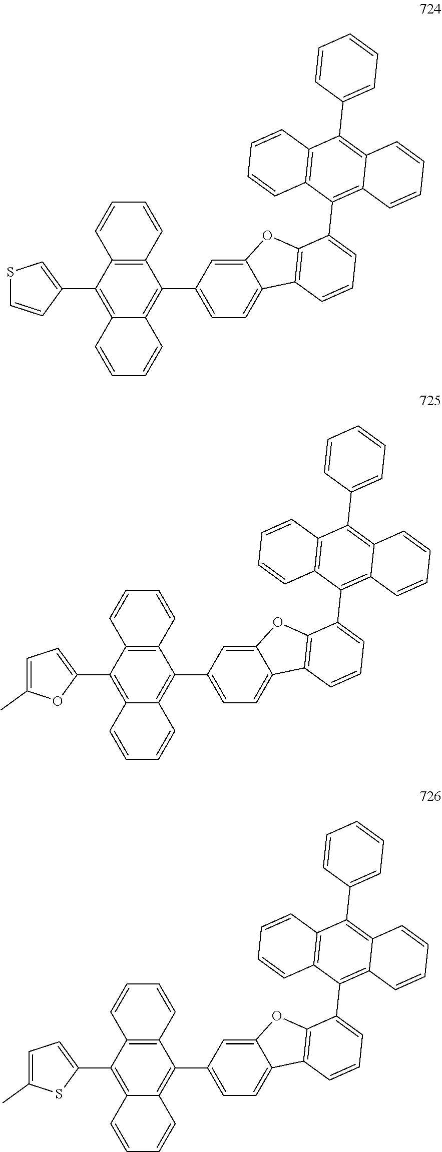 [SMILES: c1ccc(-c2c3ccccc3c(-c3cccc4c3oc3cc(-c5c6ccccc6c(-c6ccsc6)c6ccccc56)ccc34)c3ccccc23)cc1 ; Cc1ccc(-c2c3ccccc3c(-c3ccc4c(c3)oc3c(-c5c6ccccc6c(-c6ccccc6)c6ccccc56)cccc34)c3ccccc23)o1 ; Cc1ccc(-c2c3ccccc3c(-c3ccc4c(c3)oc3c(-c5c6ccccc6c(-c6ccccc6)c6ccccc56)cccc34)c3ccccc23)s1]

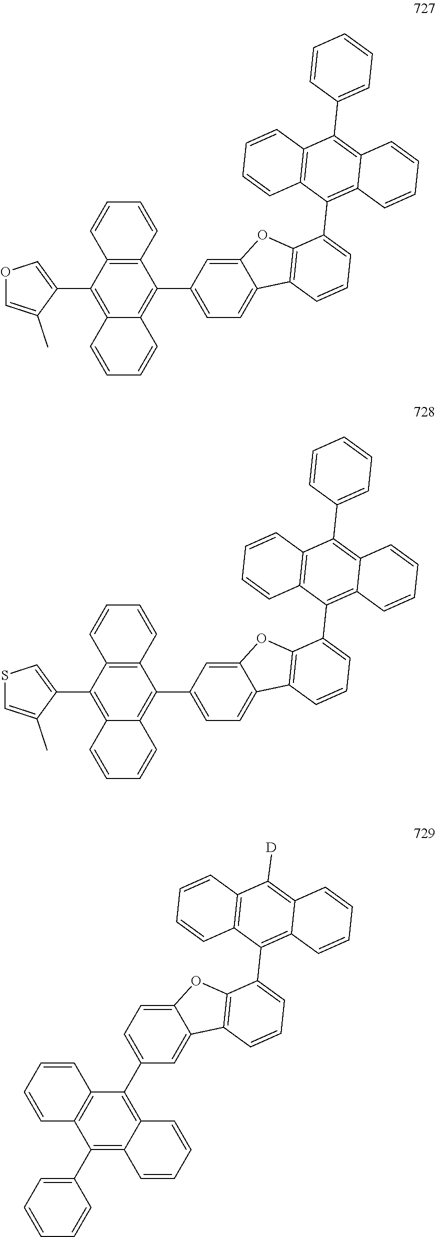 [SMILES: Cc1cocc1-c1c2ccccc2c(-c2ccc3c(c2)oc2c(-c4c5ccccc5c(-c5ccccc5)c5ccccc45)cccc23)c2ccccc12 ; Cc1cscc1-c1c2ccccc2c(-c2ccc3c(c2)oc2c(-c4c5ccccc5c(-c5ccccc5)c5ccccc45)cccc23)c2ccccc12 ; [2H]c1c2ccccc2c(-c2cccc3c2oc2ccc(-c4c5ccccc5c(-c5ccccc5)c5ccccc45)cc23)c2ccccc12]

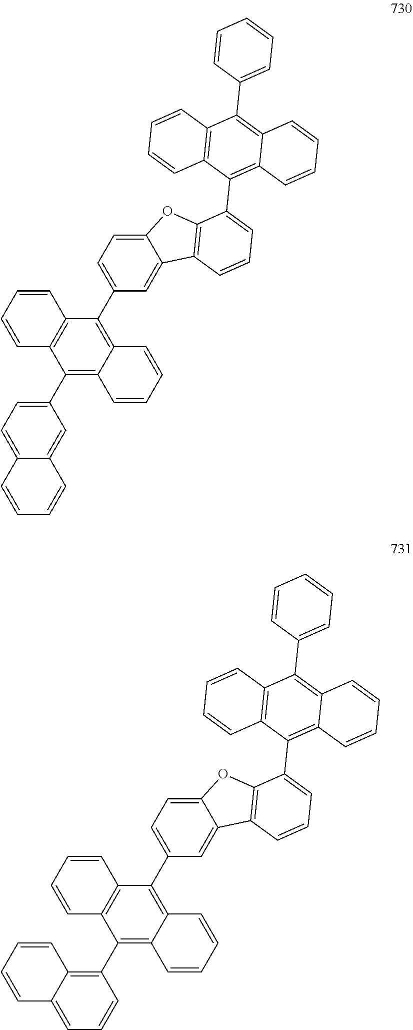 [SMILES: c1ccc(-c2c3ccccc3c(-c3cccc4c3oc3ccc(-c5c6ccccc6c(-c6ccc7ccccc7c6)c6ccccc56)cc34)c3ccccc23)cc1 ; c1ccc(-c2c3ccccc3c(-c3cccc4c3oc3ccc(-c5c6ccccc6c(-c6cccc7ccccc67)c6ccccc56)cc34)c3ccccc23)cc1]

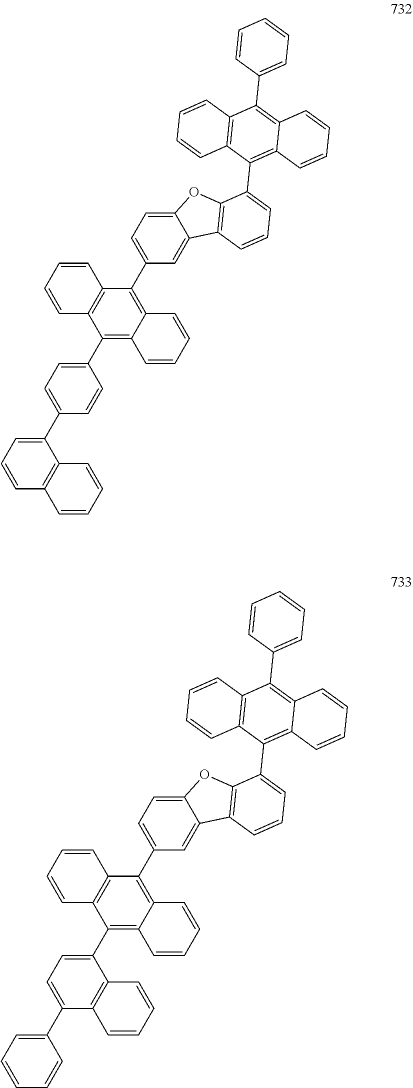 [SMILES: c1ccc(-c2c3ccccc3c(-c3cccc4c3oc3ccc(-c5c6ccccc6c(-c6ccc(-c7cccc8ccccc78)cc6)c6ccccc56)cc34)c3ccccc23)cc1 ; c1ccc(-c2ccc(-c3c4ccccc4c(-c4ccc5oc6c(-c7c8ccccc8c(-c8ccccc8)c8ccccc78)cccc6c5c4)c4ccccc34)c3ccccc23)cc1]

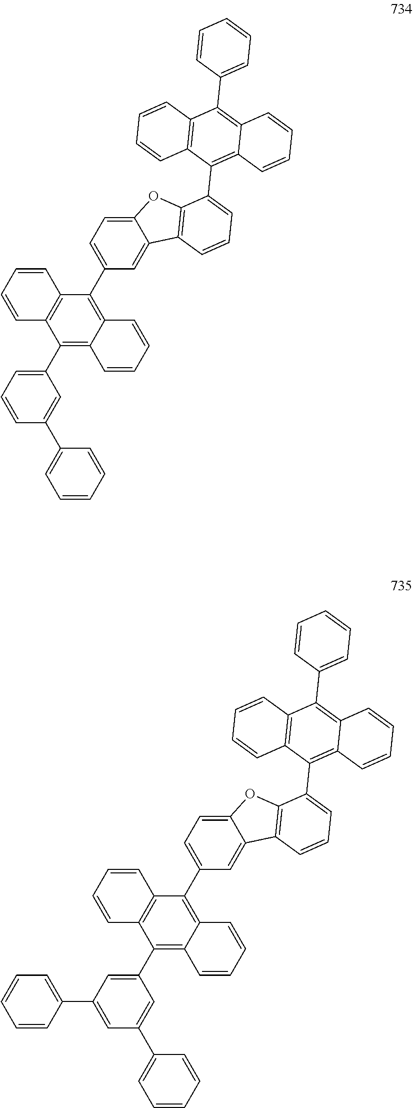 [SMILES: c1ccc(-c2cccc(-c3c4ccccc4c(-c4ccc5oc6c(-c7c8ccccc8c(-c8ccccc8)c8ccccc78)cccc6c5c4)c4ccccc34)c2)cc1 ; c1ccc(-c2cc(-c3ccccc3)cc(-c3c4ccccc4c(-c4ccc5oc6c(-c7c8ccccc8c(-c8ccccc8)c8ccccc78)cccc6c5c4)c4ccccc34)c2)cc1]

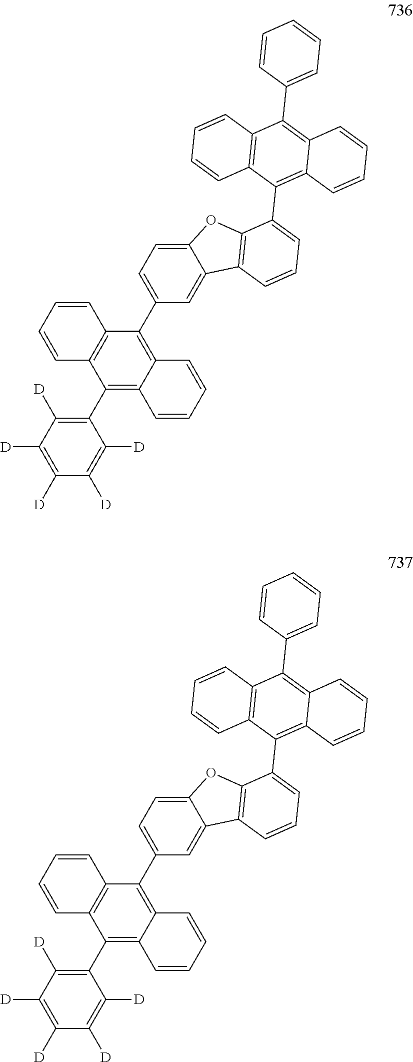 [SMILES: [2H]c1c([2H])c([2H])c(-c2c3ccccc3c(-c3ccc4oc5c(-c6c7ccccc7c(-c7ccccc7)c7ccccc67)cccc5c4c3)c3ccccc23)c([2H])c1[2H] ; [2H]c1c([2H])c([2H])c(-c2c3ccccc3c(-c3ccc4oc5c(-c6c7ccccc7c(-c7ccccc7)c7ccccc67)cccc5c4c3)c3ccccc23)c([2H])c1[2H]]

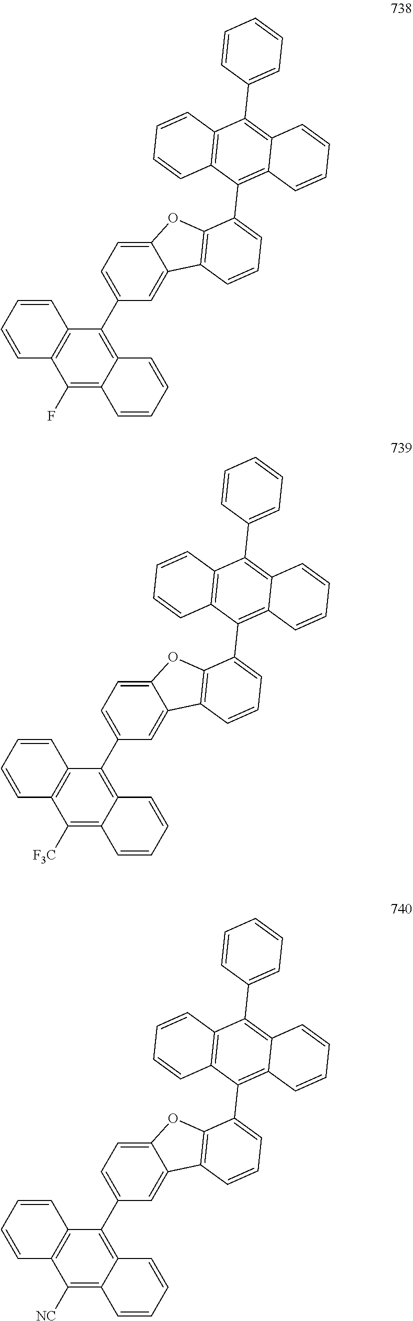 [SMILES: Fc1c2ccccc2c(-c2ccc3oc4c(-c5c6ccccc6c(-c6ccccc6)c6ccccc56)cccc4c3c2)c2ccccc12 ; FC(F)(F)c1c2ccccc2c(-c2ccc3oc4c(-c5c6ccccc6c(-c6ccccc6)c6ccccc56)cccc4c3c2)c2ccccc12 ; N#Cc1c2ccccc2c(-c2ccc3oc4c(-c5c6ccccc6c(-c6ccccc6)c6ccccc56)cccc4c3c2)c2ccccc12]

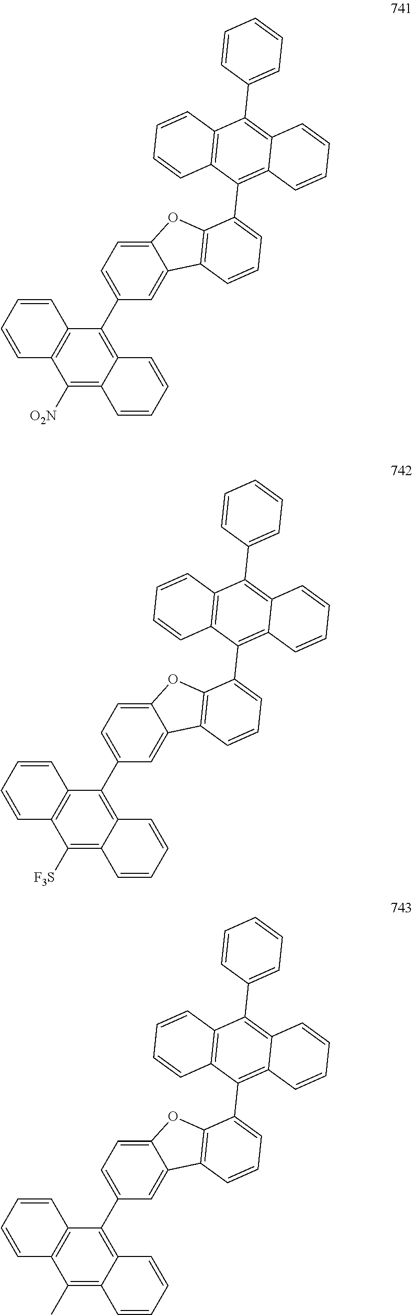

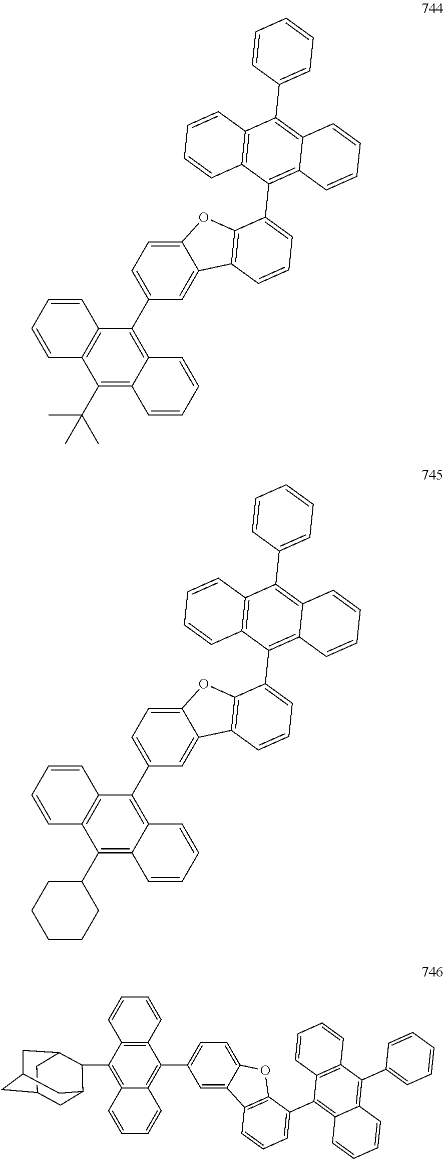 [SMILES: CC(C)(C)c1c2ccccc2c(-c2ccc3oc4c(-c5c6ccccc6c(-c6ccccc6)c6ccccc56)cccc4c3c2)c2ccccc12 ; c1ccc(-c2c3ccccc3c(-c3cccc4c3oc3ccc(-c5c6ccccc6c(C6CCCCC6)c6ccccc56)cc34)c3ccccc23)cc1 ; c1ccc(-c2c3ccccc3c(-c3cccc4c3oc3ccc(-c5c6ccccc6c(C6C7CC8CC(C7)CC6C8)c6ccccc56)cc34)c3ccccc23)cc1]

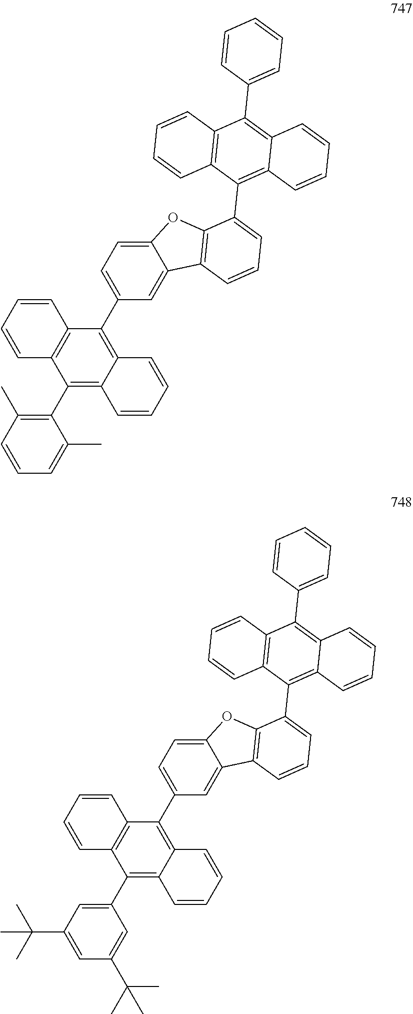 [SMILES: Cc1cccc(C)c1-c1c2ccccc2c(-c2ccc3oc4c(-c5c6ccccc6c(-c6ccccc6)c6ccccc56)cccc4c3c2)c2ccccc12 ; CC(C)(C)c1cc(-c2c3ccccc3c(-c3ccc4oc5c(-c6c7ccccc7c(-c7ccccc7)c7ccccc67)cccc5c4c3)c3ccccc23)cc(C(C)(C)C)c1]

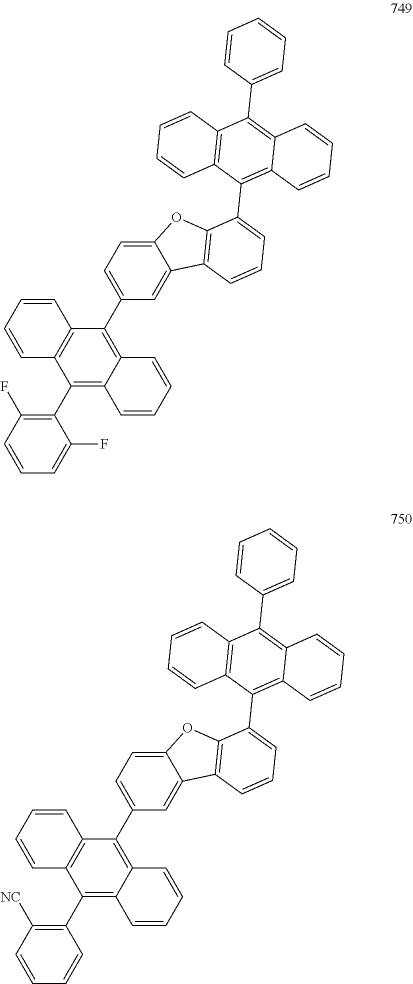 [SMILES: Fc1cccc(F)c1-c1c2ccccc2c(-c2ccc3oc4c(-c5c6ccccc6c(-c6ccccc6)c6ccccc56)cccc4c3c2)c2ccccc12 ; N#Cc1ccccc1-c1c2ccccc2c(-c2ccc3oc4c(-c5c6ccccc6c(-c6ccccc6)c6ccccc56)cccc4c3c2)c2ccccc12]

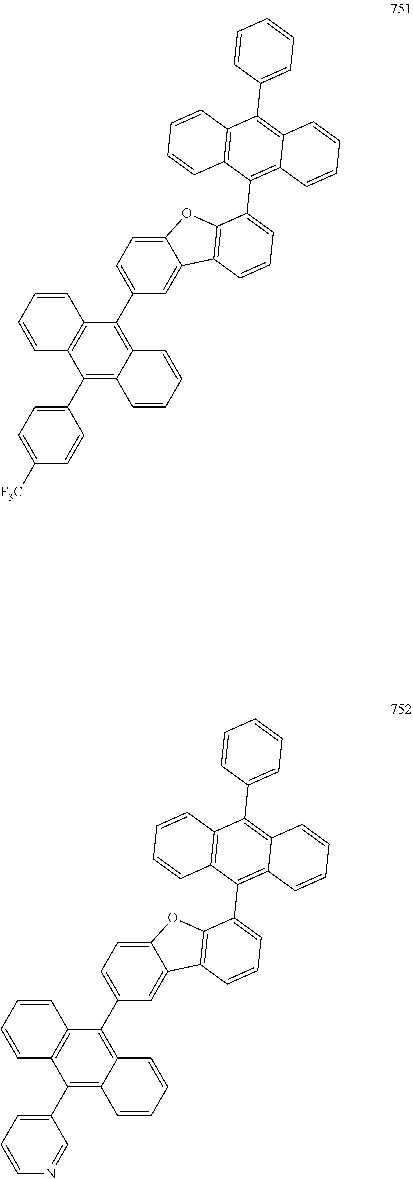 [SMILES: FC(F)(F)c1ccc(-c2c3ccccc3c(-c3ccc4oc5c(-c6c7ccccc7c(-c7ccccc7)c7ccccc67)cccc5c4c3)c3ccccc23)cc1 ; c1ccc(-c2c3ccccc3c(-c3cccc4c3oc3ccc(-c5c6ccccc6c(-c6cccnc6)c6ccccc56)cc34)c3ccccc23)cc1]

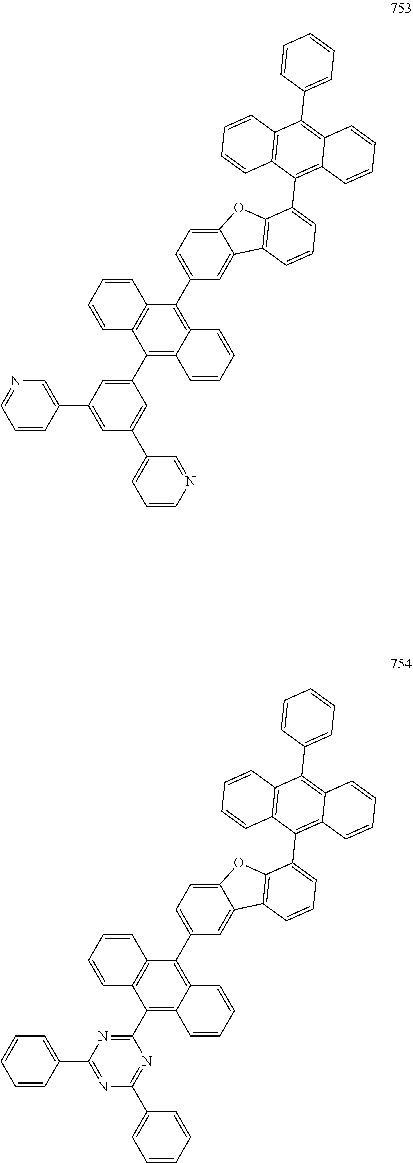 [SMILES: c1ccc(-c2c3ccccc3c(-c3cccc4c3oc3ccc(-c5c6ccccc6c(-c6cc(-c7cccnc7)cc(-c7cccnc7)c6)c6ccccc56)cc34)c3ccccc23)cc1 ; c1ccc(-c2nc(-c3ccccc3)nc(-c3c4ccccc4c(-c4ccc5oc6c(-c7c8ccccc8c(-c8ccccc8)c8ccccc78)cccc6c5c4)c4ccccc34)n2)cc1]

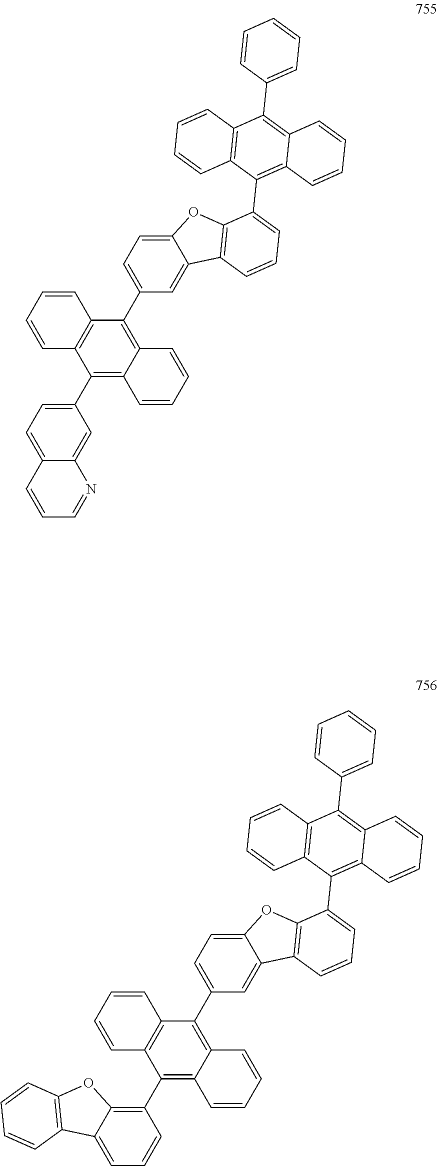 [SMILES: c1ccc(-c2c3ccccc3c(-c3cccc4c3oc3ccc(-c5c6ccccc6c(-c6ccc7cccnc7c6)c6ccccc56)cc34)c3ccccc23)cc1 ; c1ccc(-c2c3ccccc3c(-c3cccc4c3oc3ccc(-c5c6ccccc6c(-c6cccc7c6oc6ccccc67)c6ccccc56)cc34)c3ccccc23)cc1]

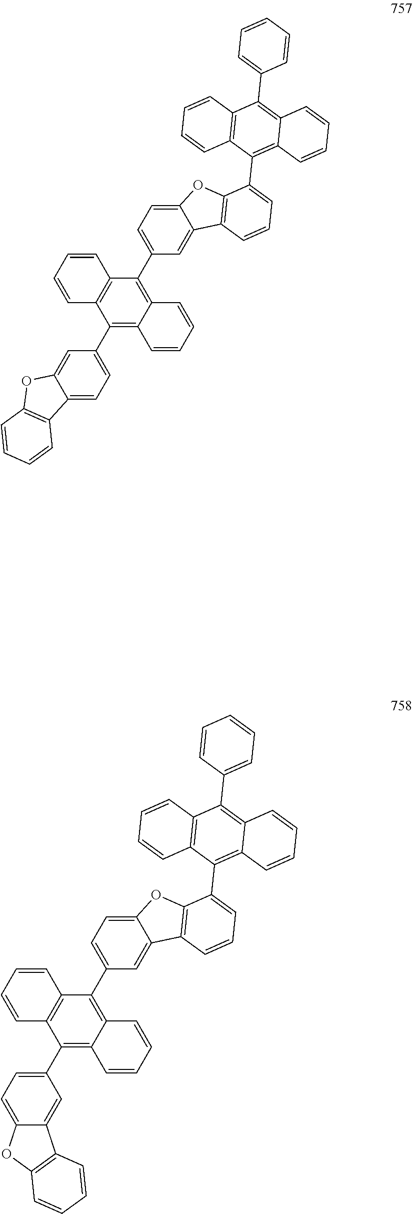 [SMILES: c1ccc(-c2c3ccccc3c(-c3cccc4c3oc3ccc(-c5c6ccccc6c(-c6ccc7c(c6)oc6ccccc67)c6ccccc56)cc34)c3ccccc23)cc1 ; c1ccc(-c2c3ccccc3c(-c3cccc4c3oc3ccc(-c5c6ccccc6c(-c6ccc7oc8ccccc8c7c6)c6ccccc56)cc34)c3ccccc23)cc1]

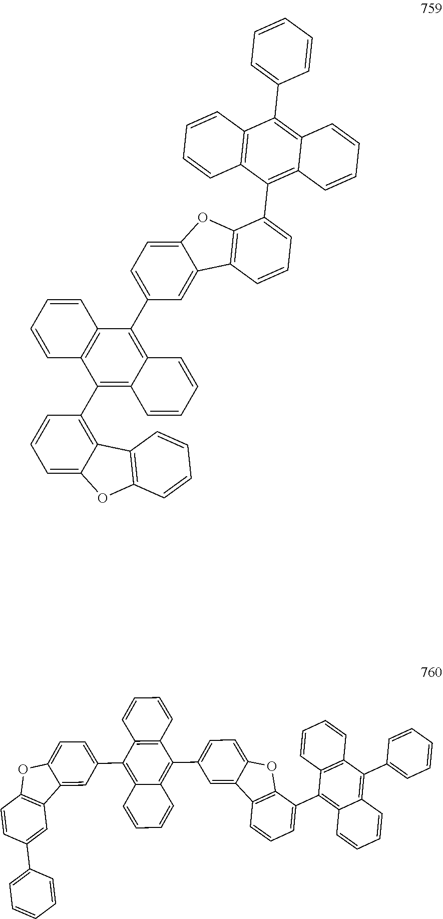 [SMILES: c1ccc(-c2c3ccccc3c(-c3cccc4c3oc3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1]

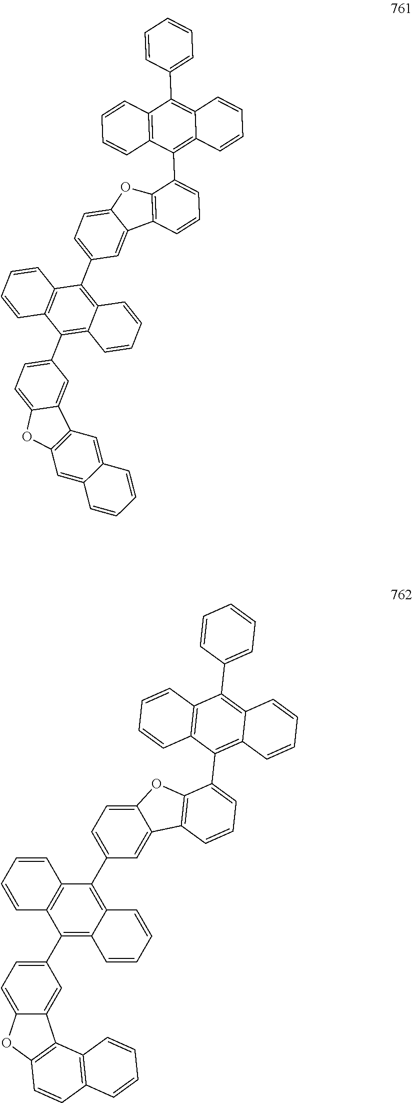 [SMILES: c1ccc(-c2c3ccccc3c(-c3cccc4c3oc3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3cccc4c3oc3ccc(-c5c6ccccc6c(-c6ccc7oc8ccc9ccccc9c8c7c6)c6ccccc56)cc34)c3ccccc23)cc1]

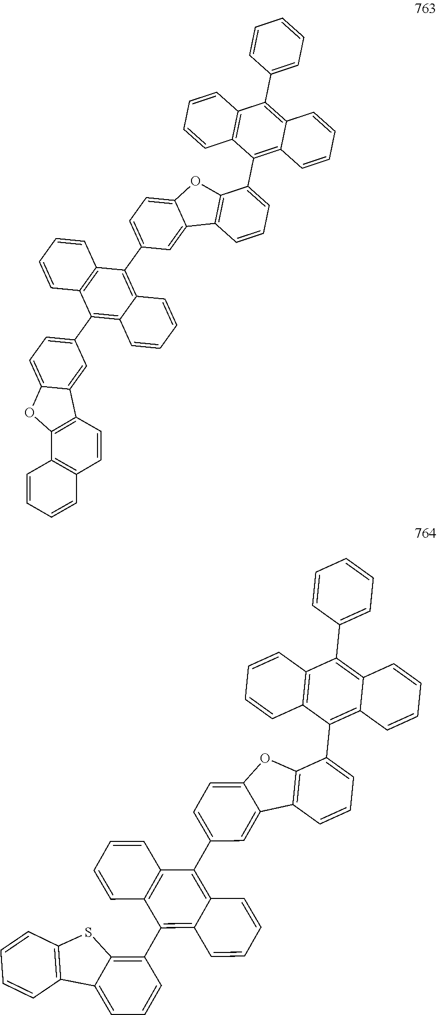 [SMILES: c1ccc(-c2c3ccccc3c(-c3cccc4c3oc3ccc(-c5c6ccccc6c(-c6ccc7oc8c9ccccc9ccc8c7c6)c6ccccc56)cc34)c3ccccc23)cc1 ; c1ccc(-c2c3ccccc3c(-c3cccc4c3oc3ccc(-c5c6ccccc6c(-c6cccc7c6sc6ccccc67)c6ccccc56)cc34)c3ccccc23)cc1]

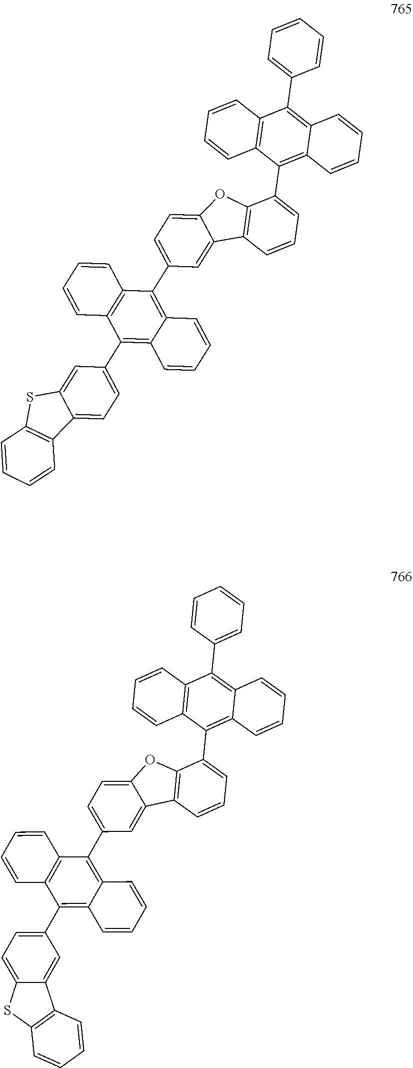 [SMILES: c1ccc(-c2c3ccccc3c(-c3cccc4c3oc3ccc(-c5c6ccccc6c(-c6ccc7c(c6)sc6ccccc67)c6ccccc56)cc34)c3ccccc23)cc1 ; c1ccc(-c2c3ccccc3c(-c3cccc4c3oc3ccc(-c5c6ccccc6c(-c6ccc7sc8ccccc8c7c6)c6ccccc56)cc34)c3ccccc23)cc1]

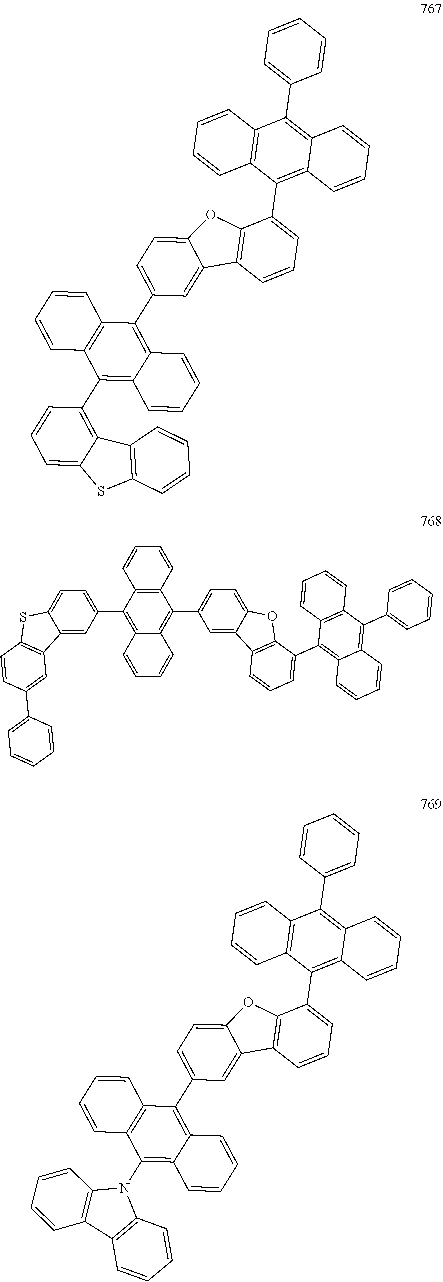 [SMILES: c1ccc(-c2c3ccccc3c(-c3cccc4c3oc3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3oc3ccc(-c5c6ccccc6c(-n6c7ccccc7c7ccccc76)c6ccccc56)cc34)c3ccccc23)cc1]

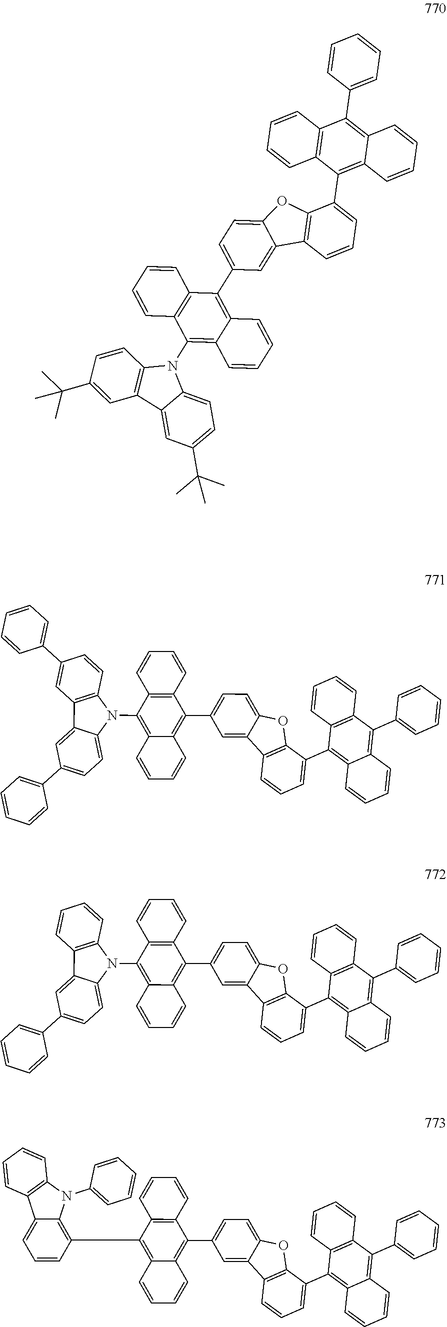 [SMILES: CC(C)(C)c1ccc2c(c1)c1cc(C(C)(C)C)ccc1n2-c1c2ccccc2c(-c2ccc3oc4c(-c5c6ccccc6c(-c6ccccc6)c6ccccc56)cccc4c3c2)c2ccccc12 ; c1ccc(-c2ccc3c(c2)c2cc(-c4ccccc4)ccc2n3-c2c3ccccc3c(-c3ccc4oc5c(-c6c7ccccc7c(-c7ccccc7)c7ccccc67)cccc5c4c3)c3ccccc23)cc1 ; c1ccc(-c2ccc3c(c2)c2ccccc2n3-c2c3ccccc3c(-c3ccc4oc5c(-c6c7ccccc7c(-c7ccccc7)c7ccccc67)cccc5c4c3)c3ccccc23)cc1 ; c1ccc(-c2c3ccccc3c(-c3cccc4c3oc3ccc(-c5c6ccccc6c(-c6cccc7c8ccccc8n(-c8ccccc8)c67)c6ccccc56)cc34)c3ccccc23)cc1]

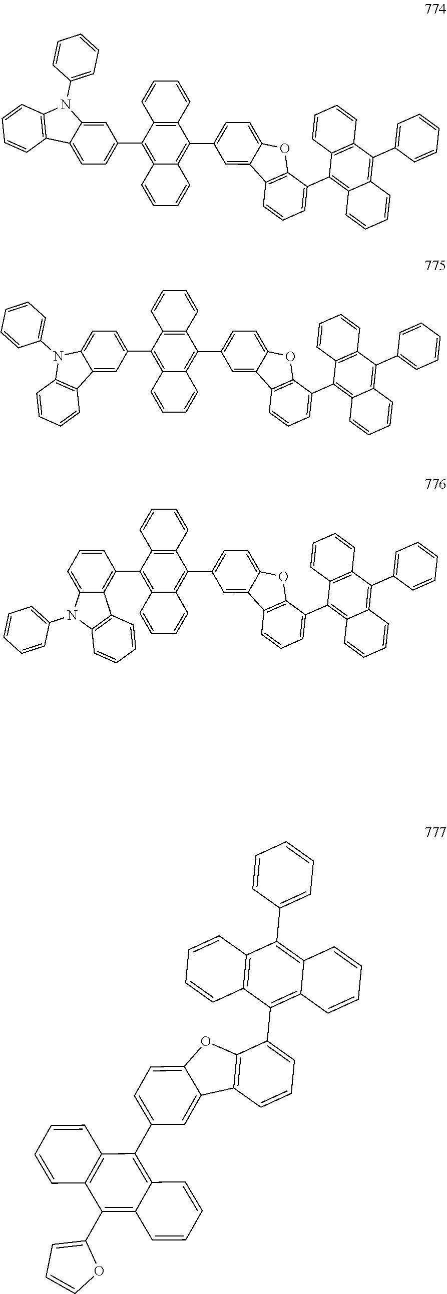 [SMILES: c1ccc(-c2c3ccccc3c(-c3cccc4c3oc3ccc(-c5c6ccccc6c(-c6ccc7c8ccccc8n(-c8ccccc8)c7c6)c6ccccc56)cc34)c3ccccc23)cc1 ; c1ccc(-c2c3ccccc3c(-c3cccc4c3oc3ccc(-c5c6ccccc6c(-c6ccc7c(c6)c6ccccc6n7-c6ccccc6)c6ccccc56)cc34)c3ccccc23)cc1 ; c1ccc(-c2c3ccccc3c(-c3cccc4c3oc3ccc(-c5c6ccccc6c(-c6cccc7c6c6ccccc6n7-c6ccccc6)c6ccccc56)cc34)c3ccccc23)cc1 ; c1ccc(-c2c3ccccc3c(-c3cccc4c3oc3ccc(-c5c6ccccc6c(-c6ccco6)c6ccccc56)cc34)c3ccccc23)cc1]

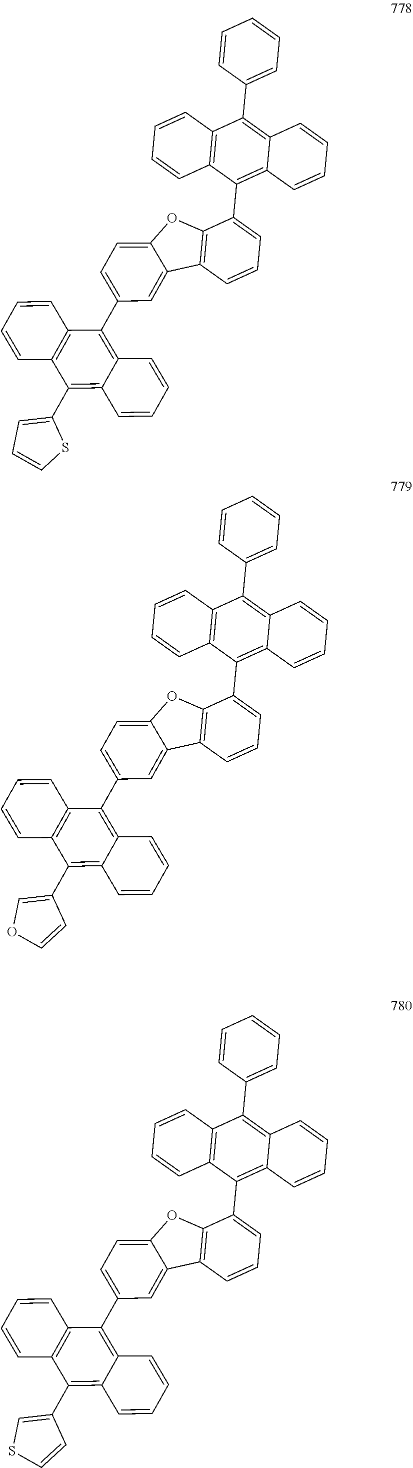 [SMILES: c1ccc(-c2c3ccccc3c(-c3cccc4c3oc3ccc(-c5c6ccccc6c(-c6cccs6)c6ccccc56)cc34)c3ccccc23)cc1 ; c1ccc(-c2c3ccccc3c(-c3cccc4c3oc3ccc(-c5c6ccccc6c(-c6ccoc6)c6ccccc56)cc34)c3ccccc23)cc1 ; c1ccc(-c2c3ccccc3c(-c3cccc4c3oc3ccc(-c5c6ccccc6c(-c6ccsc6)c6ccccc56)cc34)c3ccccc23)cc1]

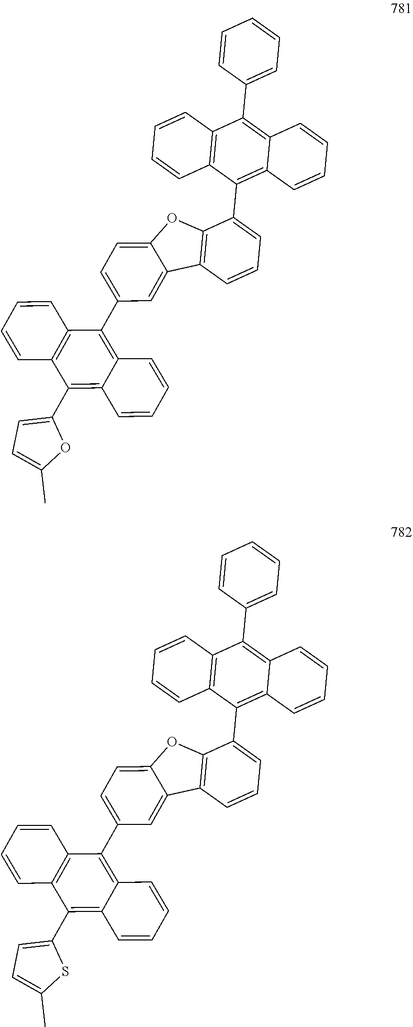 [SMILES: Cc1ccc(-c2c3ccccc3c(-c3ccc4oc5c(-c6c7ccccc7c(-c7ccccc7)c7ccccc67)cccc5c4c3)c3ccccc23)o1 ; Cc1ccc(-c2c3ccccc3c(-c3ccc4oc5c(-c6c7ccccc7c(-c7ccccc7)c7ccccc67)cccc5c4c3)c3ccccc23)s1]

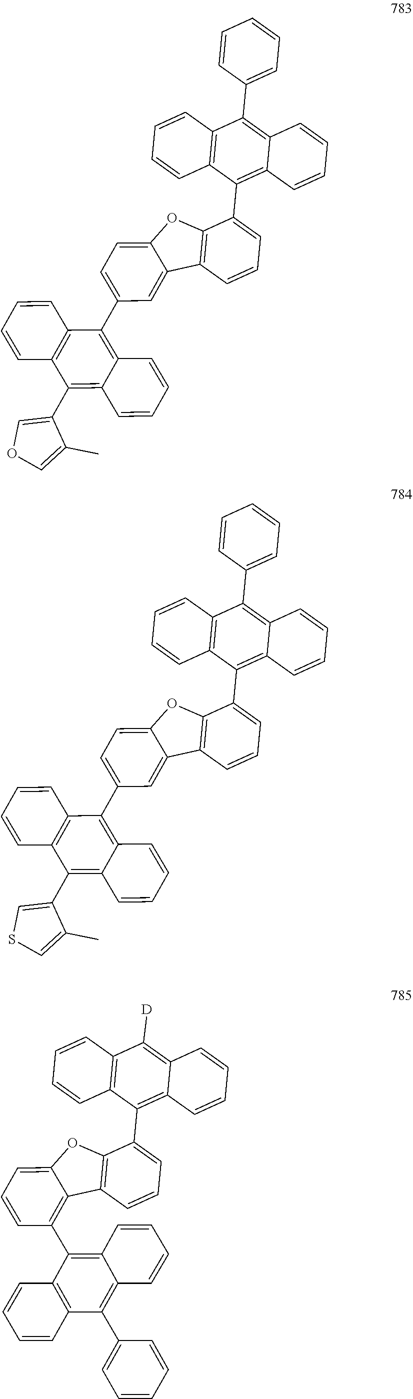 [SMILES: Cc1cocc1-c1c2ccccc2c(-c2ccc3oc4c(-c5c6ccccc6c(-c6ccccc6)c6ccccc56)cccc4c3c2)c2ccccc12 ; Cc1cscc1-c1c2ccccc2c(-c2ccc3oc4c(-c5c6ccccc6c(-c6ccccc6)c6ccccc56)cccc4c3c2)c2ccccc12 ; [2H]c1c2ccccc2c(-c2cccc3c2oc2cccc(-c4c5ccccc5c(-c5ccccc5)c5ccccc45)c23)c2ccccc12]

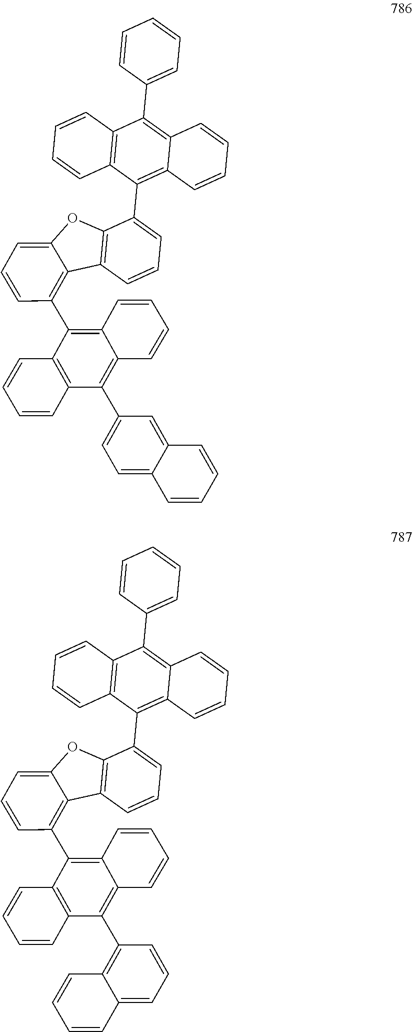 [SMILES: c1ccc(-c2c3ccccc3c(-c3cccc4c3oc3cccc(-c5c6ccccc6c(-c6ccc7ccccc7c6)c6ccccc56)c34)c3ccccc23)cc1 ; c1ccc(-c2c3ccccc3c(-c3cccc4c3oc3cccc(-c5c6ccccc6c(-c6cccc7ccccc67)c6ccccc56)c34)c3ccccc23)cc1]

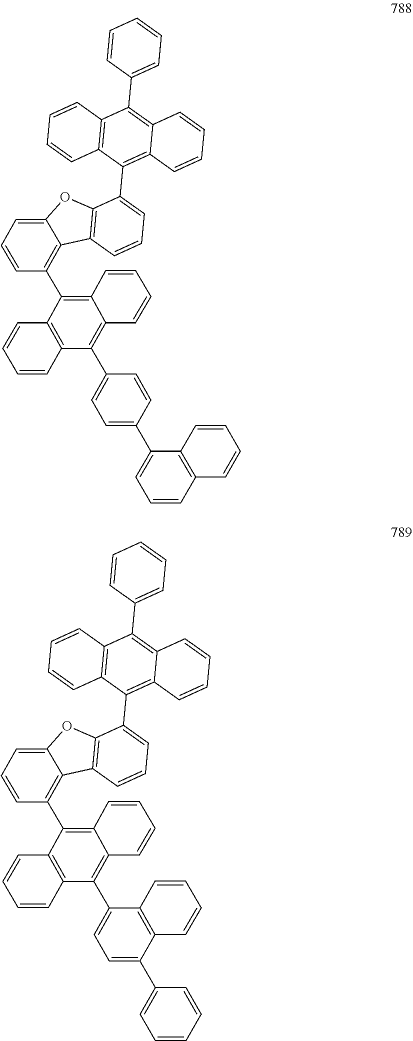

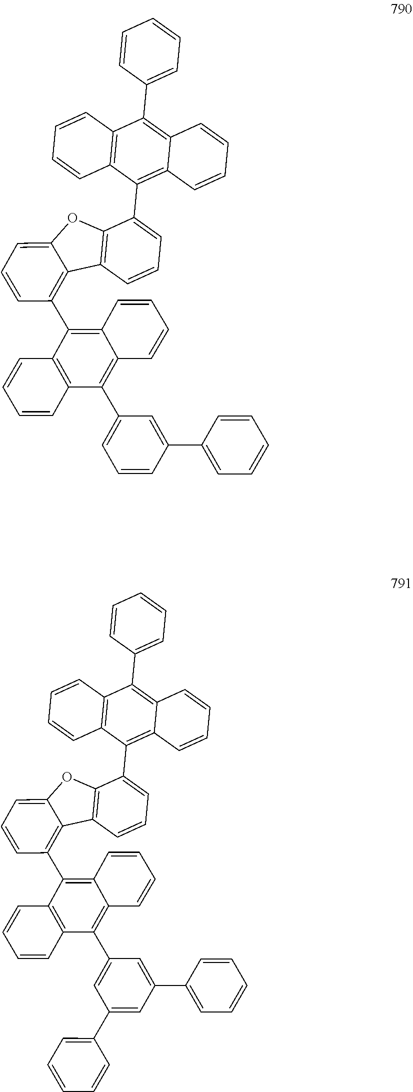 [SMILES: c1ccc(-c2cccc(-c3c4ccccc4c(-c4cccc5oc6c(-c7c8ccccc8c(-c8ccccc8)c8ccccc78)cccc6c45)c4ccccc34)c2)cc1 ; c1ccc(-c2cc(-c3ccccc3)cc(-c3c4ccccc4c(-c4cccc5oc6c(-c7c8ccccc8c(-c8ccccc8)c8ccccc78)cccc6c45)c4ccccc34)c2)cc1]

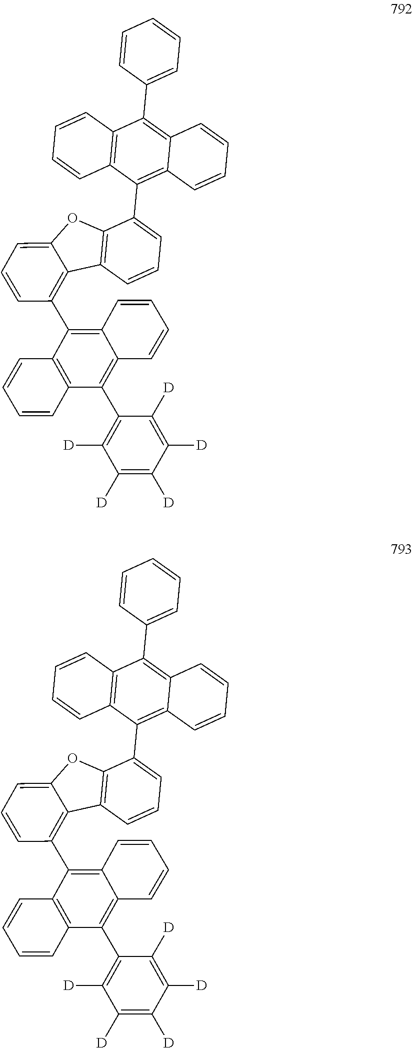 [SMILES: [2H]c1c([2H])c([2H])c(-c2c3ccccc3c(-c3cccc4oc5c(-c6c7ccccc7c(-c7ccccc7)c7ccccc67)cccc5c34)c3ccccc23)c([2H])c1[2H] ; [2H]c1c([2H])c([2H])c(-c2c3ccccc3c(-c3cccc4oc5c(-c6c7ccccc7c(-c7ccccc7)c7ccccc67)cccc5c34)c3ccccc23)c([2H])c1[2H]]

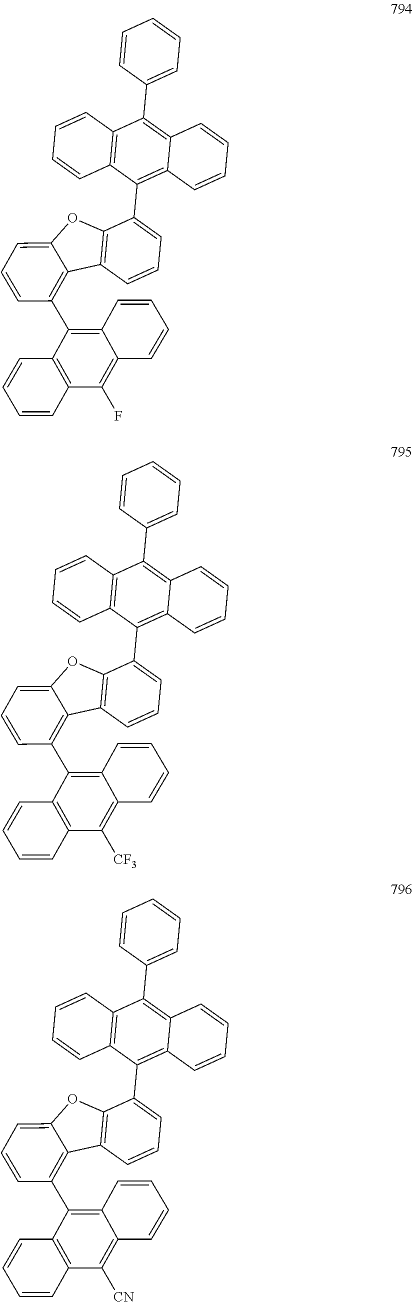 [SMILES: Fc1c2ccccc2c(-c2cccc3oc4c(-c5c6ccccc6c(-c6ccccc6)c6ccccc56)cccc4c23)c2ccccc12 ; FC(F)(F)c1c2ccccc2c(-c2cccc3oc4c(-c5c6ccccc6c(-c6ccccc6)c6ccccc56)cccc4c23)c2ccccc12 ; N#Cc1c2ccccc2c(-c2cccc3oc4c(-c5c6ccccc6c(-c6ccccc6)c6ccccc56)cccc4c23)c2ccccc12]

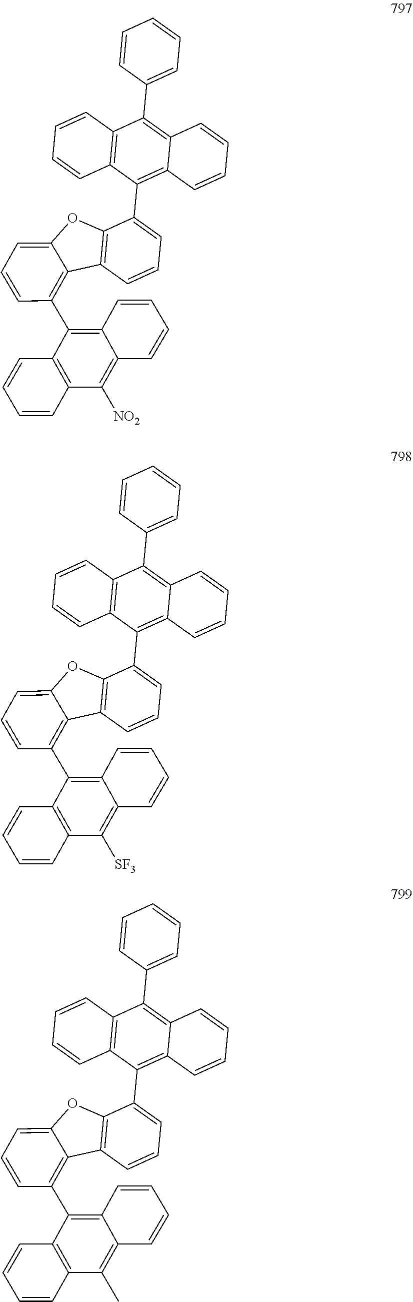

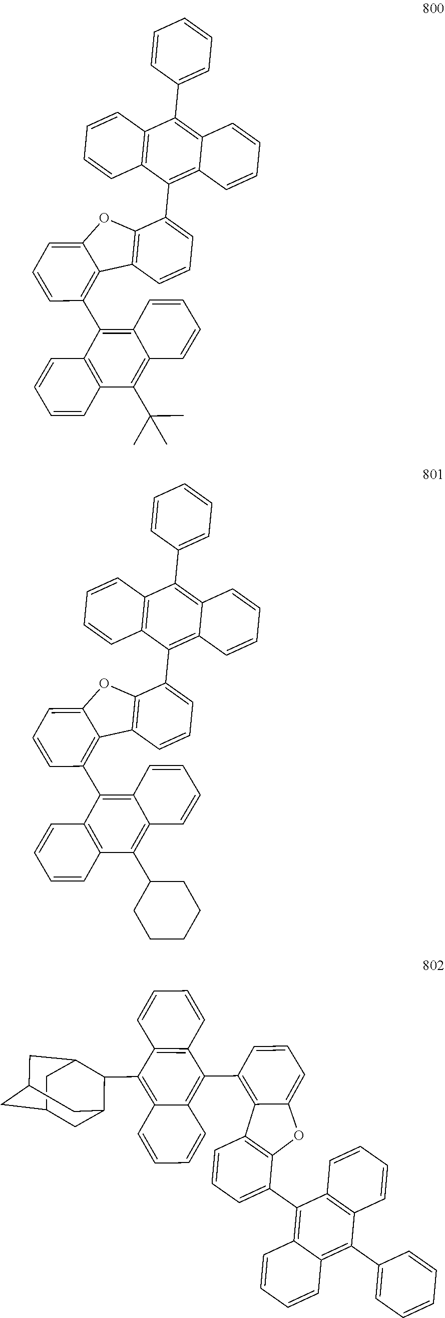

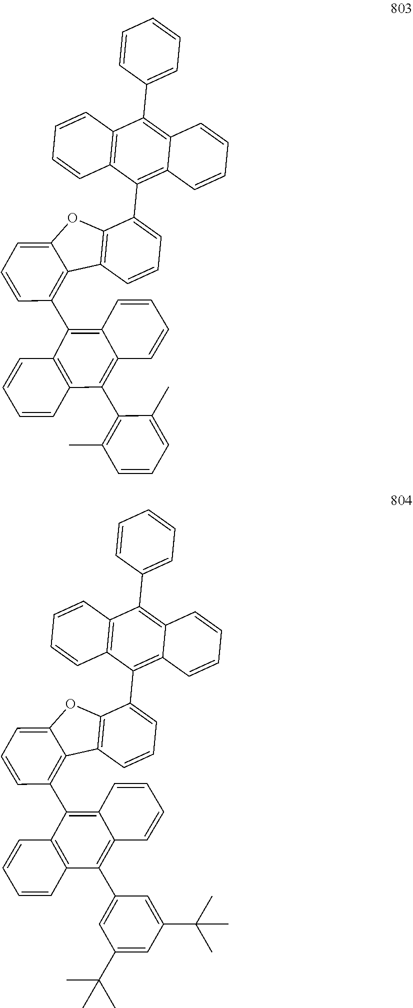

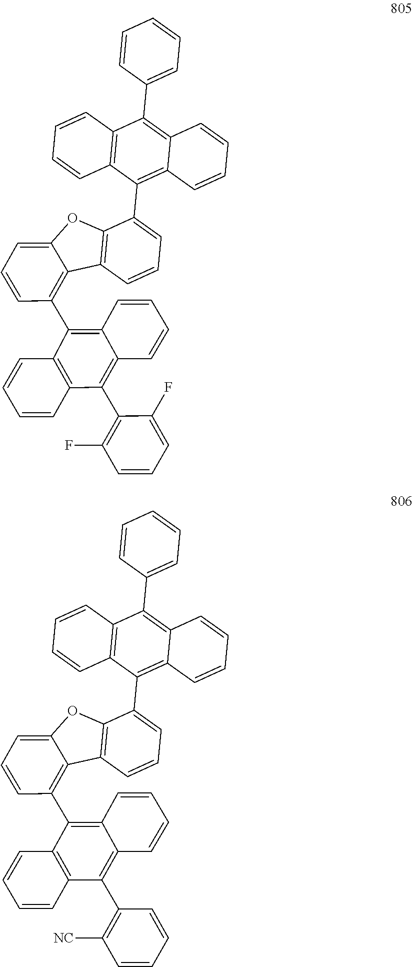

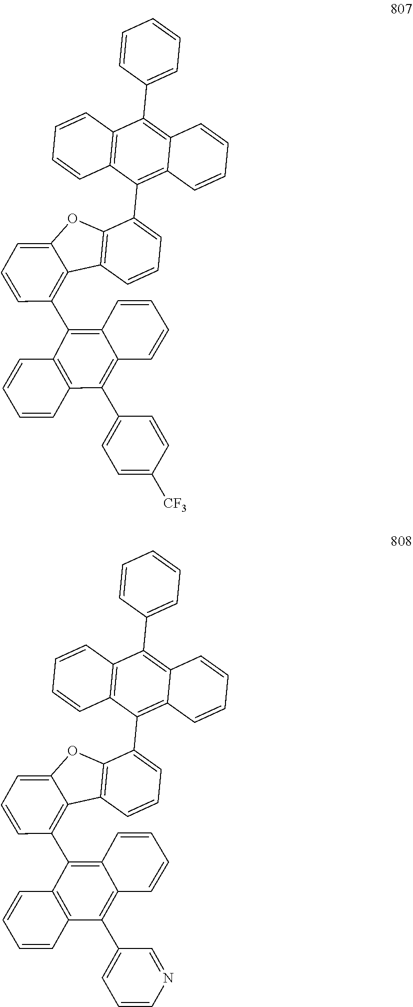 [SMILES: FC(F)(F)c1ccc(-c2c3ccccc3c(-c3cccc4oc5c(-c6c7ccccc7c(-c7ccccc7)c7ccccc67)cccc5c34)c3ccccc23)cc1 ; c1ccc(-c2c3ccccc3c(-c3cccc4c3oc3cccc(-c5c6ccccc6c(-c6cccnc6)c6ccccc56)c34)c3ccccc23)cc1]

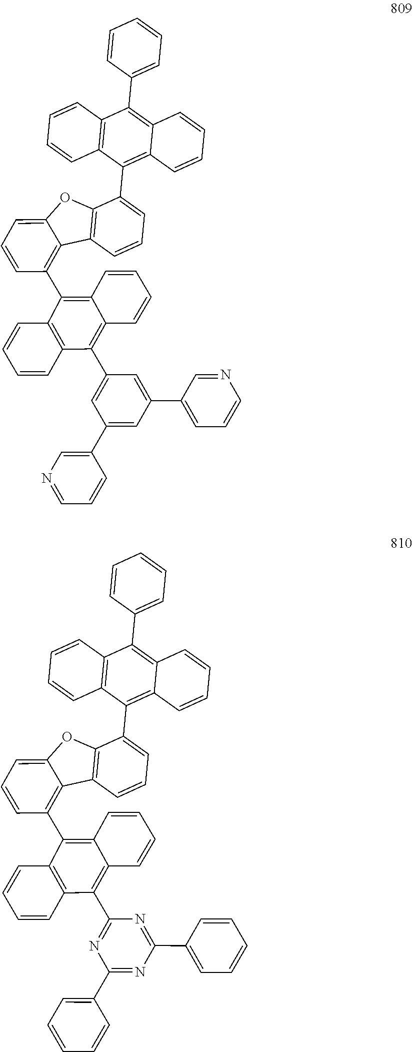 [SMILES: c1ccc(-c2c3ccccc3c(-c3cccc4c3oc3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5oc6c(-c7c8ccccc8c(-c8ccccc8)c8ccccc78)cccc6c45)c4ccccc34)n2)cc1]

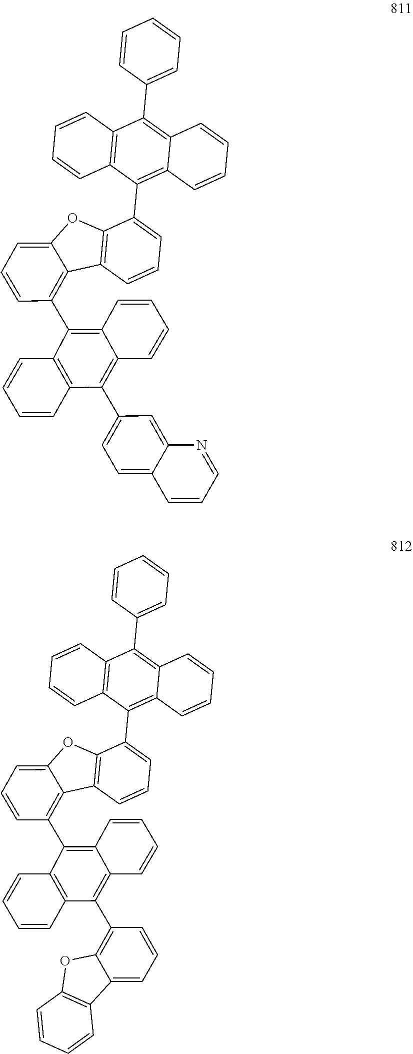 [SMILES: c1ccc(-c2c3ccccc3c(-c3cccc4c3oc3cccc(-c5c6ccccc6c(-c6ccc7cccnc7c6)c6ccccc56)c34)c3ccccc23)cc1 ; c1ccc(-c2c3ccccc3c(-c3cccc4c3oc3cccc(-c5c6ccccc6c(-c6cccc7c6oc6ccccc67)c6ccccc56)c34)c3ccccc23)cc1]

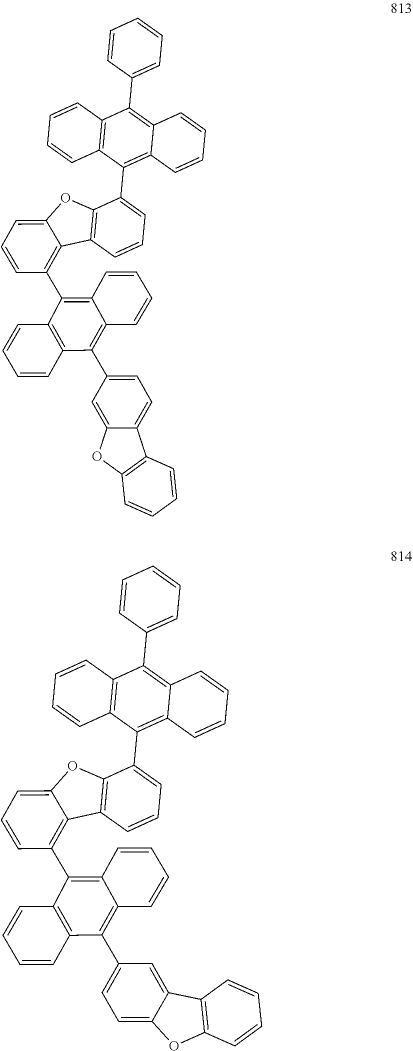 [SMILES: c1ccc(-c2c3ccccc3c(-c3cccc4c3oc3cccc(-c5c6ccccc6c(-c6ccc7c(c6)oc6ccccc67)c6ccccc56)c34)c3ccccc23)cc1 ; c1ccc(-c2c3ccccc3c(-c3cccc4c3oc3cccc(-c5c6ccccc6c(-c6ccc7oc8ccccc8c7c6)c6ccccc56)c34)c3ccccc23)cc1]

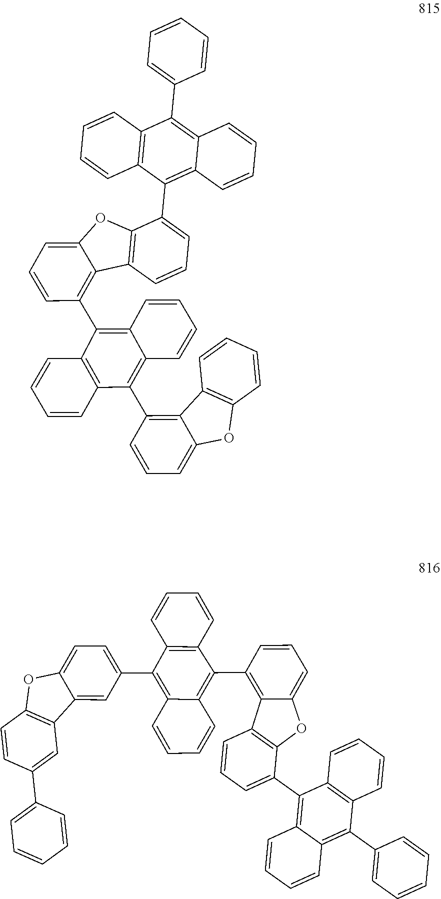 [SMILES: c1ccc(-c2c3ccccc3c(-c3cccc4c3oc3cccc(-c5c6ccccc6c(-c6cccc7oc8ccccc8c67)c6ccccc56)c34)c3ccccc23)cc1 ; c1ccc(-c2ccc3oc4ccc(-c5c6ccccc6c(-c6cccc7oc8c(-c9c%10ccccc%10c(-c%10ccccc%10)c%10ccccc9%10)cccc8c67)c6ccccc56)cc4c3c2)cc1]

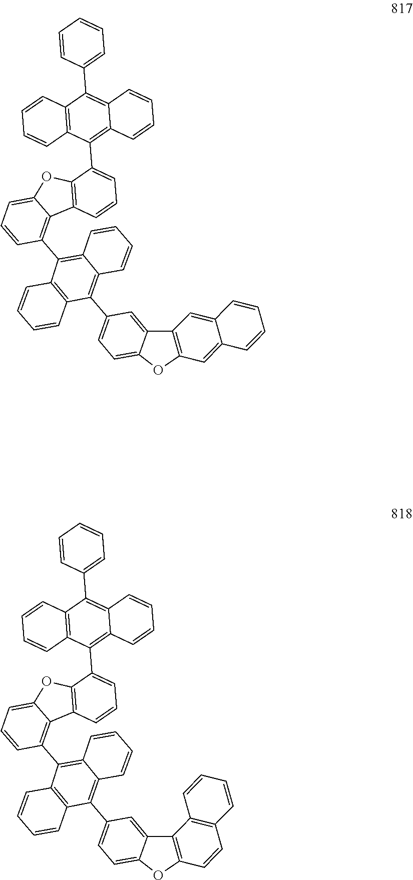 [SMILES: c1ccc(-c2c3ccccc3c(-c3cccc4c3oc3cccc(-c5c6ccccc6c(-c6ccc7oc8cc9ccccc9cc8c7c6)c6ccccc56)c34)c3ccccc23)cc1 ; c1ccc(-c2c3ccccc3c(-c3cccc4c3oc3cccc(-c5c6ccccc6c(-c6ccc7oc8ccc9ccccc9c8c7c6)c6ccccc56)c34)c3ccccc23)cc1]

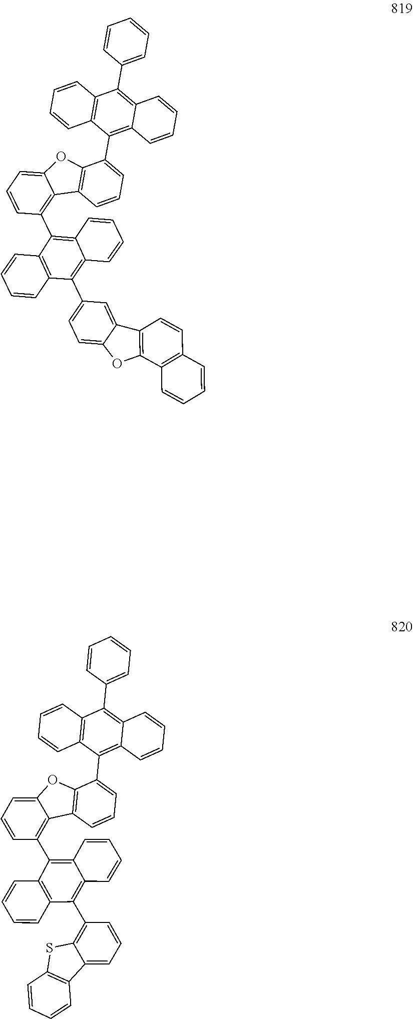 [SMILES: c1ccc(-c2c3ccccc3c(-c3cccc4c3oc3cccc(-c5c6ccccc6c(-c6ccc7oc8c9ccccc9ccc8c7c6)c6ccccc56)c34)c3ccccc23)cc1 ; c1ccc(-c2c3ccccc3c(-c3cccc4c3oc3cccc(-c5c6ccccc6c(-c6cccc7c6sc6ccccc67)c6ccccc56)c34)c3ccccc23)cc1]

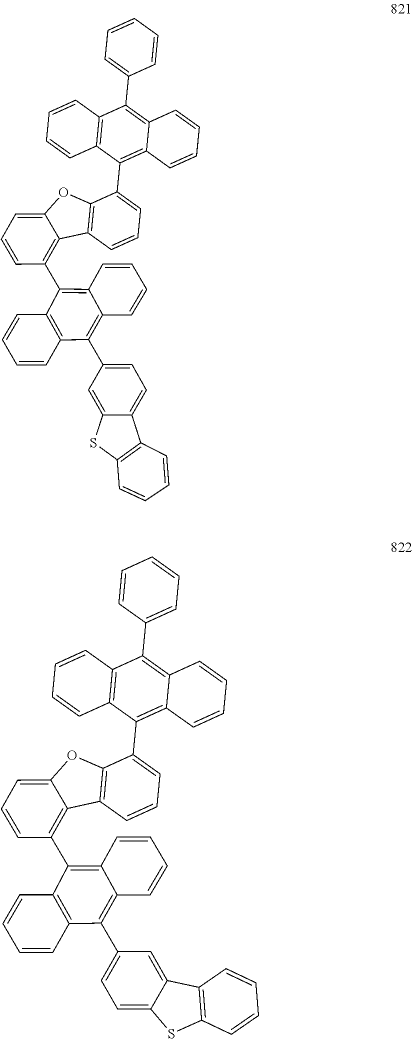 [SMILES: c1ccc(-c2c3ccccc3c(-c3cccc4c3oc3cccc(-c5c6ccccc6c(-c6ccc7c(c6)sc6ccccc67)c6ccccc56)c34)c3ccccc23)cc1 ; c1ccc(-c2c3ccccc3c(-c3cccc4c3oc3cccc(-c5c6ccccc6c(-c6ccc7sc8ccccc8c7c6)c6ccccc56)c34)c3ccccc23)cc1]

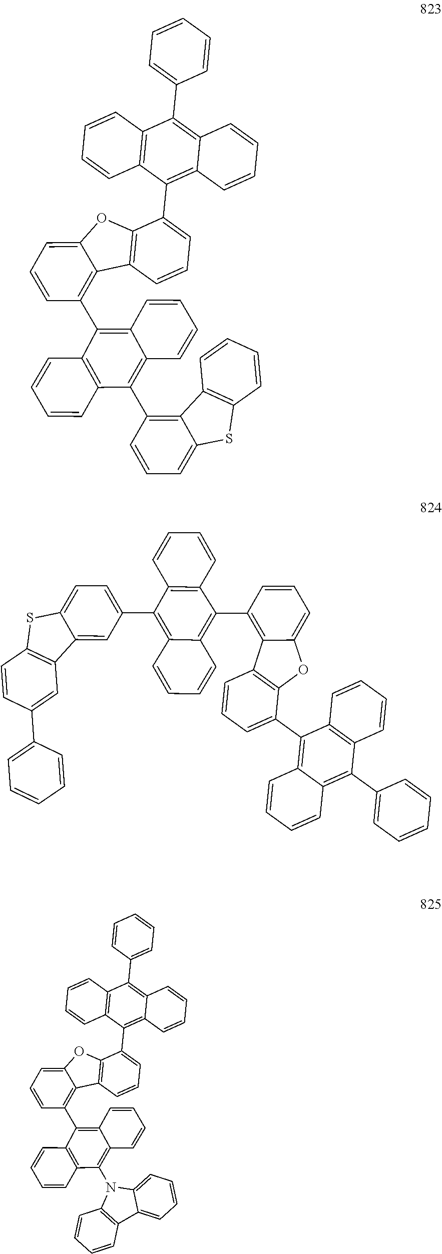 [SMILES: c1ccc(-c2c3ccccc3c(-c3cccc4c3oc3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7oc8c(-c9c%10ccccc%10c(-c%10ccccc%10)c%10ccccc9%10)cccc8c67)c6ccccc56)cc4c3c2)cc1 ; c1ccc(-c2c3ccccc3c(-c3cccc4c3oc3cccc(-c5c6ccccc6c(-n6c7ccccc7c7ccccc76)c6ccccc56)c34)c3ccccc23)cc1]

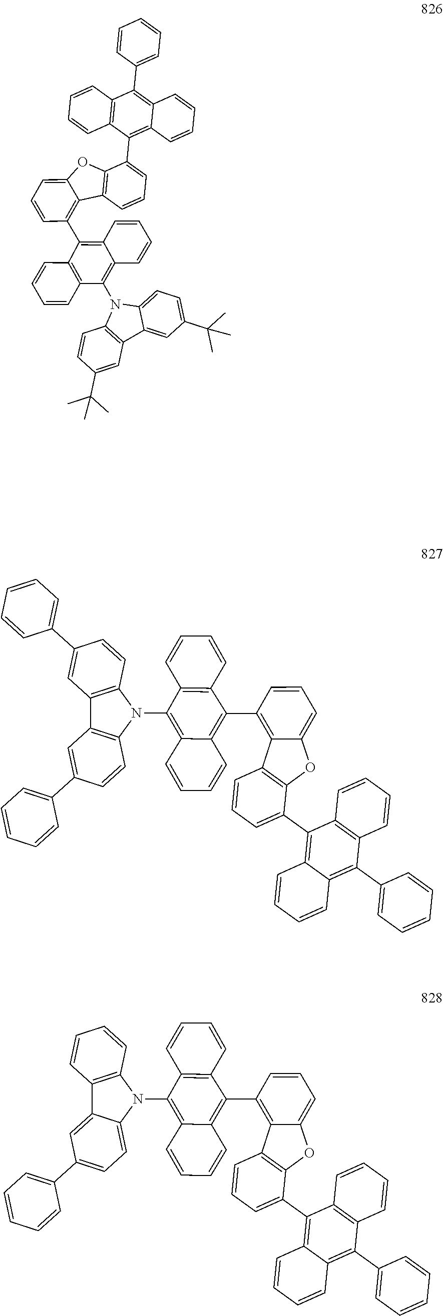 [SMILES: CC(C)(C)c1ccc2c(c1)c1cc(C(C)(C)C)ccc1n2-c1c2ccccc2c(-c2cccc3oc4c(-c5c6ccccc6c(-c6ccccc6)c6ccccc56)cccc4c23)c2ccccc12 ; c1ccc(-c2ccc3c(c2)c2cc(-c4ccccc4)ccc2n3-c2c3ccccc3c(-c3cccc4oc5c(-c6c7ccccc7c(-c7ccccc7)c7ccccc67)cccc5c34)c3ccccc23)cc1 ; c1ccc(-c2ccc3c(c2)c2ccccc2n3-c2c3ccccc3c(-c3cccc4oc5c(-c6c7ccccc7c(-c7ccccc7)c7ccccc67)cccc5c34)c3ccccc23)cc1]

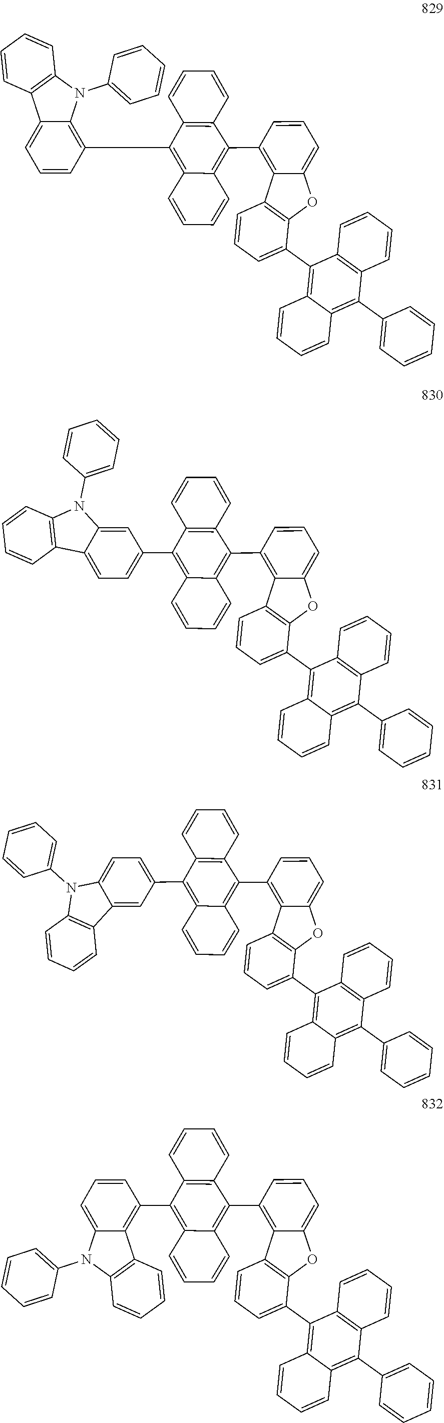 [SMILES: c1ccc(-c2c3ccccc3c(-c3cccc4c3oc3cccc(-c5c6ccccc6c(-c6cccc7c8ccccc8n(-c8ccccc8)c67)c6ccccc56)c34)c3ccccc23)cc1 ; c1ccc(-c2c3ccccc3c(-c3cccc4c3oc3cccc(-c5c6ccccc6c(-c6ccc7c8ccccc8n(-c8ccccc8)c7c6)c6ccccc56)c34)c3ccccc23)cc1 ; c1ccc(-c2c3ccccc3c(-c3cccc4c3oc3cccc(-c5c6ccccc6c(-c6ccc7c(c6)c6ccccc6n7-c6ccccc6)c6ccccc56)c34)c3ccccc23)cc1 ; c1ccc(-c2c3ccccc3c(-c3cccc4c3oc3cccc(-c5c6ccccc6c(-c6cccc7c6c6ccccc6n7-c6ccccc6)c6ccccc56)c34)c3ccccc23)cc1]

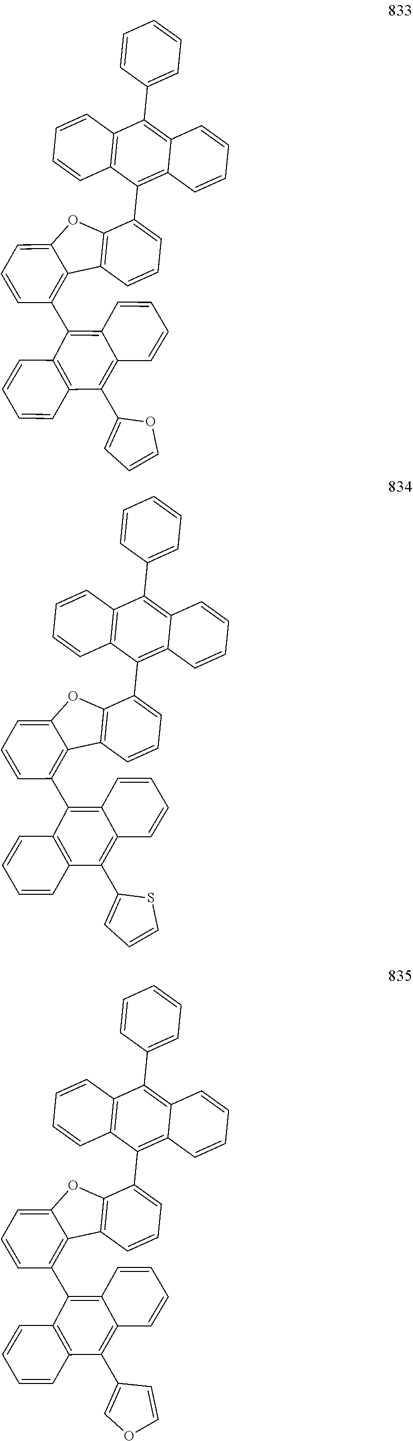

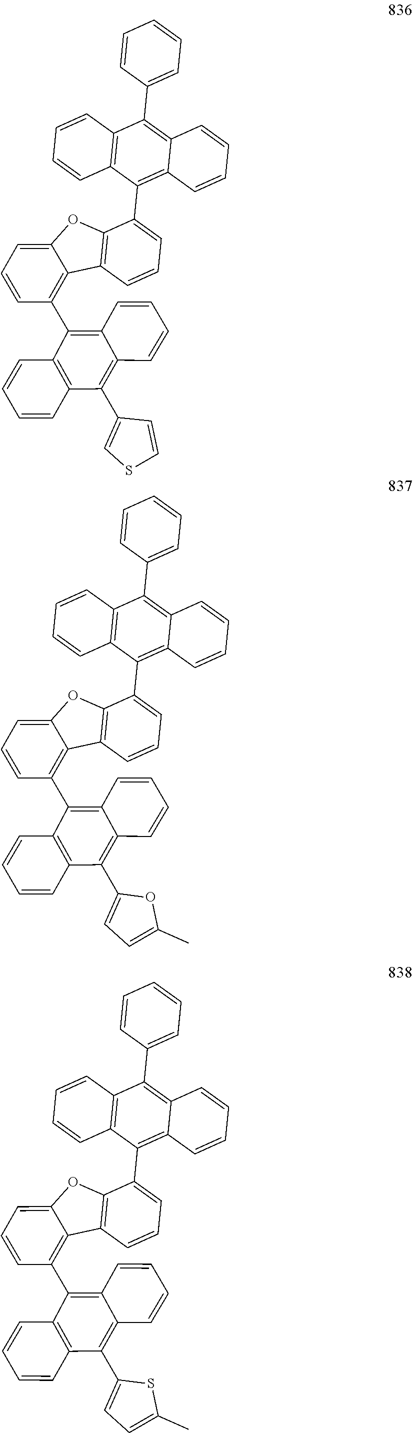

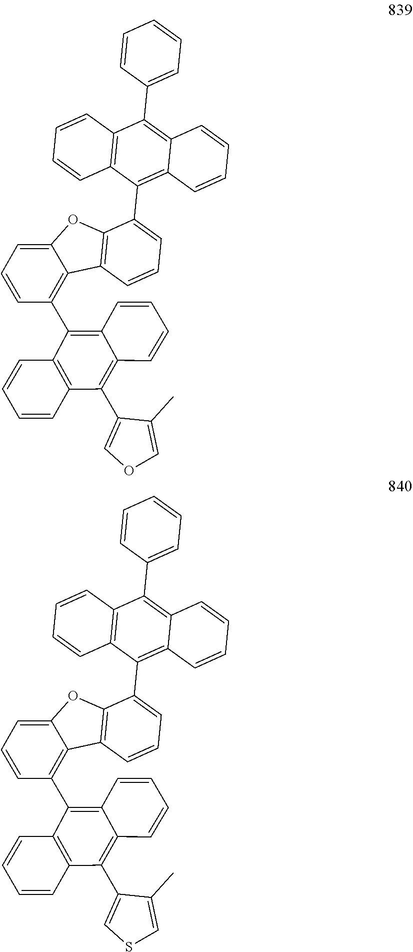

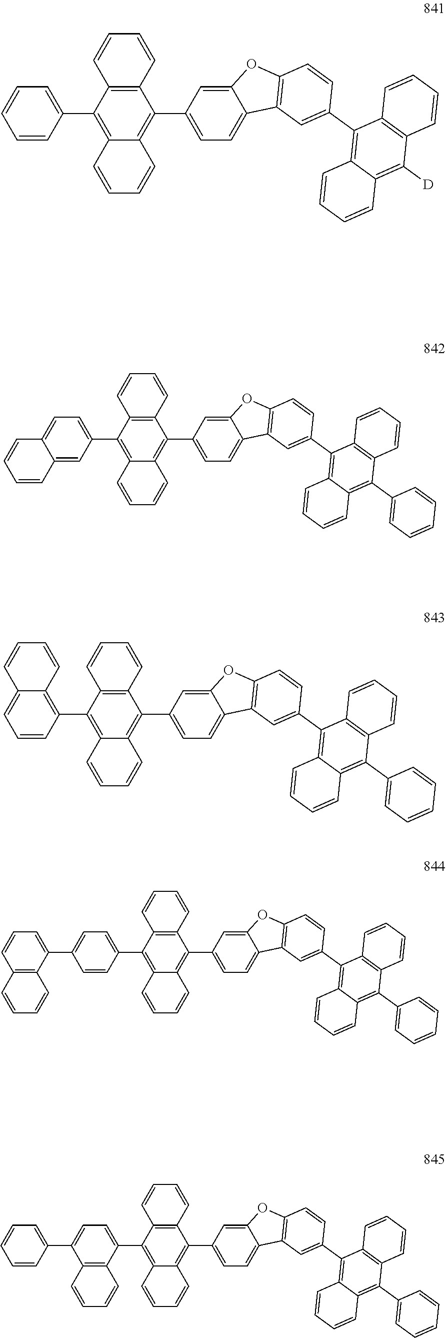 [SMILES: [2H]c1c2ccccc2c(-c2ccc3oc4cc(-c5c6ccccc6c(-c6ccccc6)c6ccccc56)ccc4c3c2)c2ccccc12 ; c1ccc(-c2c3ccccc3c(-c3ccc4oc5cc(-c6c7ccccc7c(-c7ccc8ccccc8c7)c7ccccc67)ccc5c4c3)c3ccccc23)cc1 ; c1ccc(-c2c3ccccc3c(-c3ccc4oc5cc(-c6c7ccccc7c(-c7cccc8ccccc78)c7ccccc67)ccc5c4c3)c3ccccc23)cc1 ; c1ccc(-c2c3ccccc3c(-c3ccc4oc5cc(-c6c7ccccc7c(-c7ccc(-c8cccc9ccccc89)cc7)c7ccccc67)ccc5c4c3)c3ccccc23)cc1 ; c1ccc(-c2ccc(-c3c4ccccc4c(-c4ccc5c(c4)oc4ccc(-c6c7ccccc7c(-c7ccccc7)c7ccccc67)cc45)c4ccccc34)c3ccccc23)cc1]

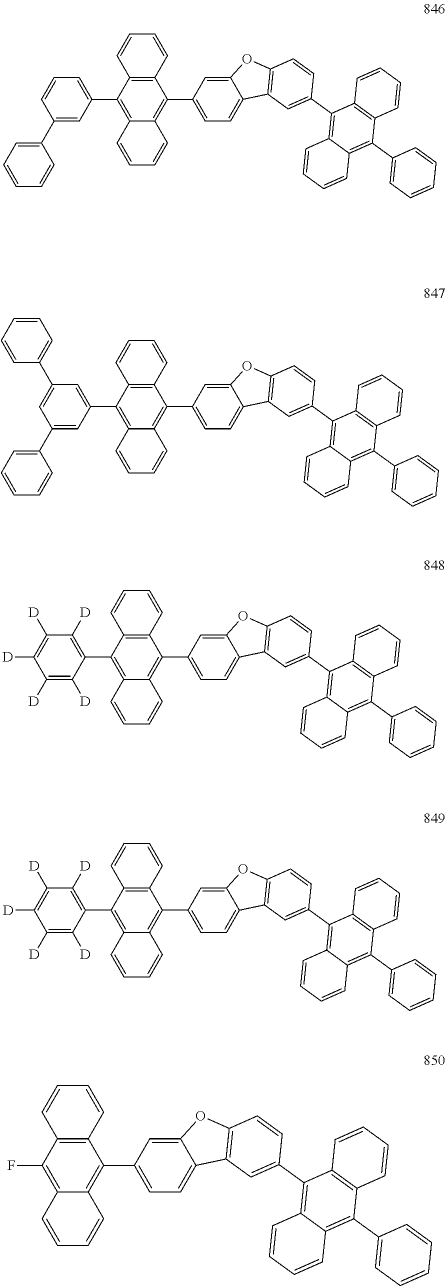

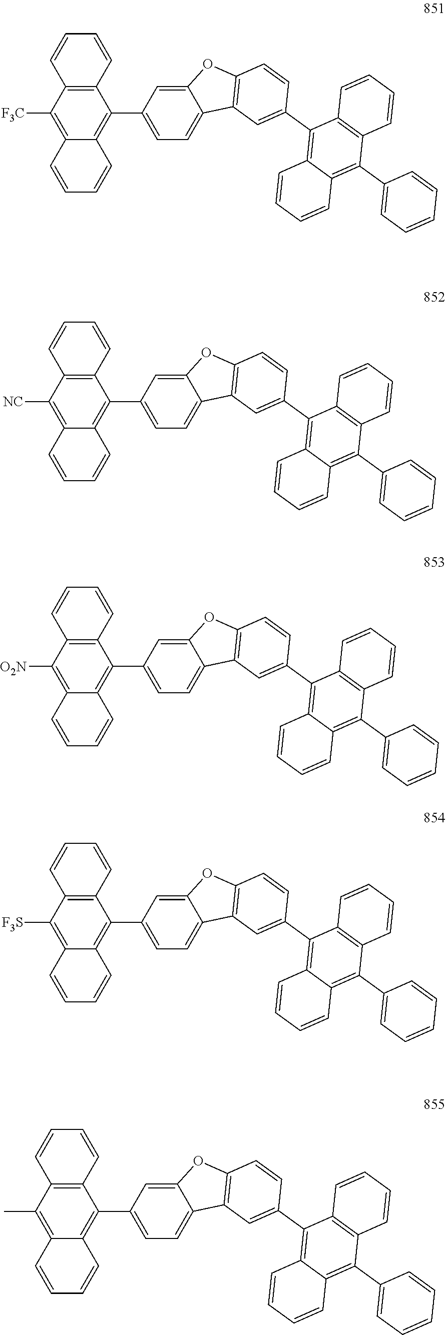

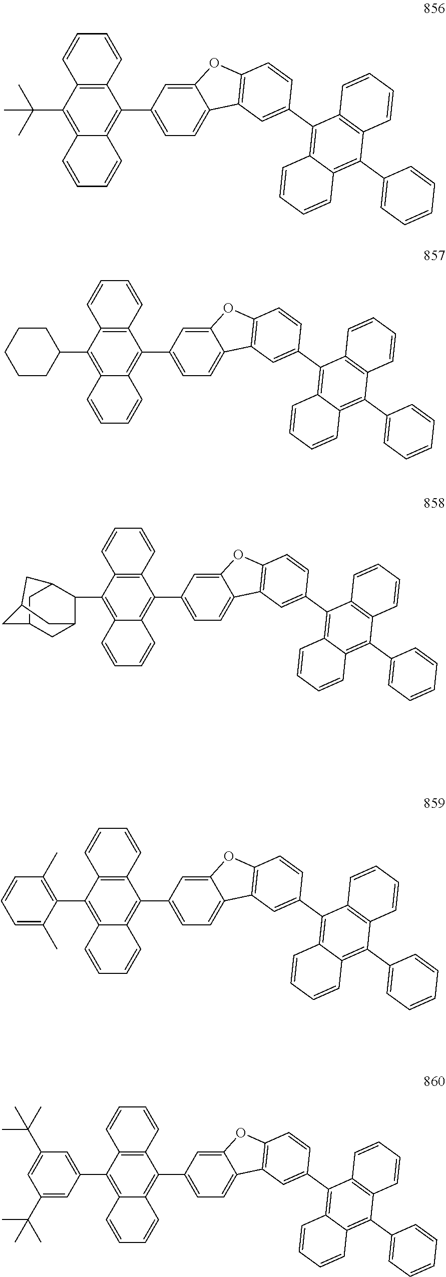 [SMILES: CC(C)(C)c1c2ccccc2c(-c2ccc3c(c2)oc2ccc(-c4c5ccccc5c(-c5ccccc5)c5ccccc45)cc23)c2ccccc12 ; c1ccc(-c2c3ccccc3c(-c3ccc4oc5cc(-c6c7ccccc7c(C7CCCCC7)c7ccccc67)ccc5c4c3)c3ccccc23)cc1 ; c1ccc(-c2c3ccccc3c(-c3ccc4oc5cc(-c6c7ccccc7c(C7C8CC9CC(C8)CC7C9)c7ccccc67)ccc5c4c3)c3ccccc23)cc1 ; Cc1cccc(C)c1-c1c2ccccc2c(-c2ccc3c(c2)oc2ccc(-c4c5ccccc5c(-c5ccccc5)c5ccccc45)cc23)c2ccccc12 ; CC(C)(C)c1cc(-c2c3ccccc3c(-c3ccc4c(c3)oc3ccc(-c5c6ccccc6c(-c6ccccc6)c6ccccc56)cc34)c3ccccc23)cc(C(C)(C)C)c1]

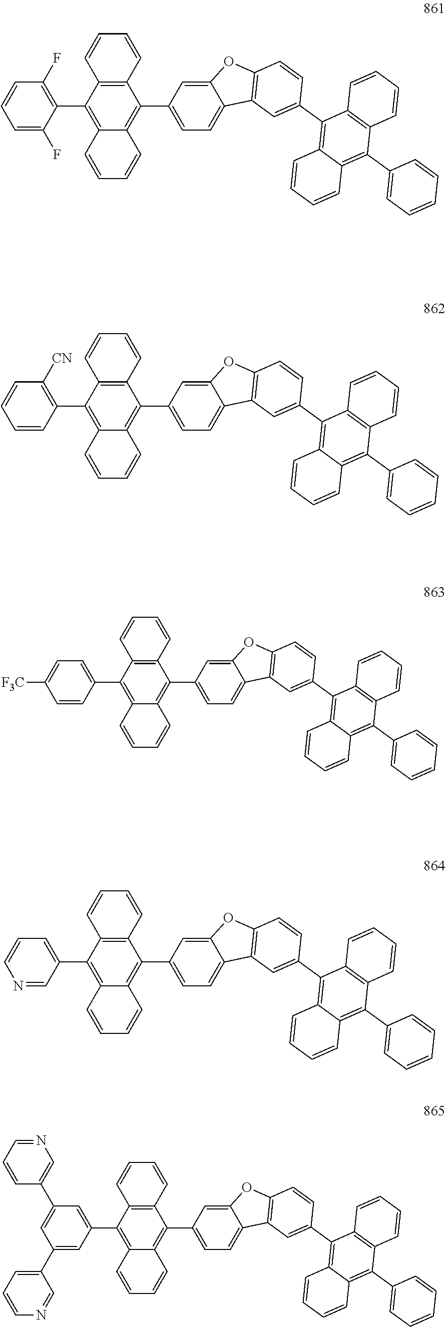 [SMILES: Fc1cccc(F)c1-c1c2ccccc2c(-c2ccc3c(c2)oc2ccc(-c4c5ccccc5c(-c5ccccc5)c5ccccc45)cc23)c2ccccc12 ; N#Cc1ccccc1-c1c2ccccc2c(-c2ccc3c(c2)oc2ccc(-c4c5ccccc5c(-c5ccccc5)c5ccccc45)cc23)c2ccccc12 ; FC(F)(F)c1ccc(-c2c3ccccc3c(-c3ccc4c(c3)oc3ccc(-c5c6ccccc6c(-c6ccccc6)c6ccccc56)cc34)c3ccccc23)cc1 ; c1ccc(-c2c3ccccc3c(-c3ccc4oc5cc(-c6c7ccccc7c(-c7cccnc7)c7ccccc67)ccc5c4c3)c3ccccc23)cc1 ; c1ccc(-c2c3ccccc3c(-c3ccc4oc5cc(-c6c7ccccc7c(-c7cc(-c8cccnc8)cc(-c8cccnc8)c7)c7ccccc67)ccc5c4c3)c3ccccc23)cc1]

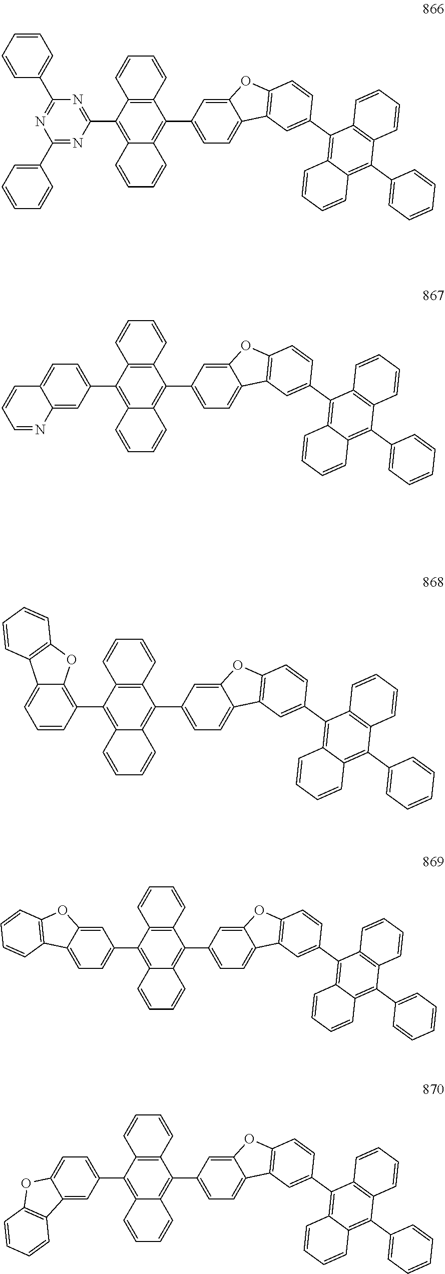

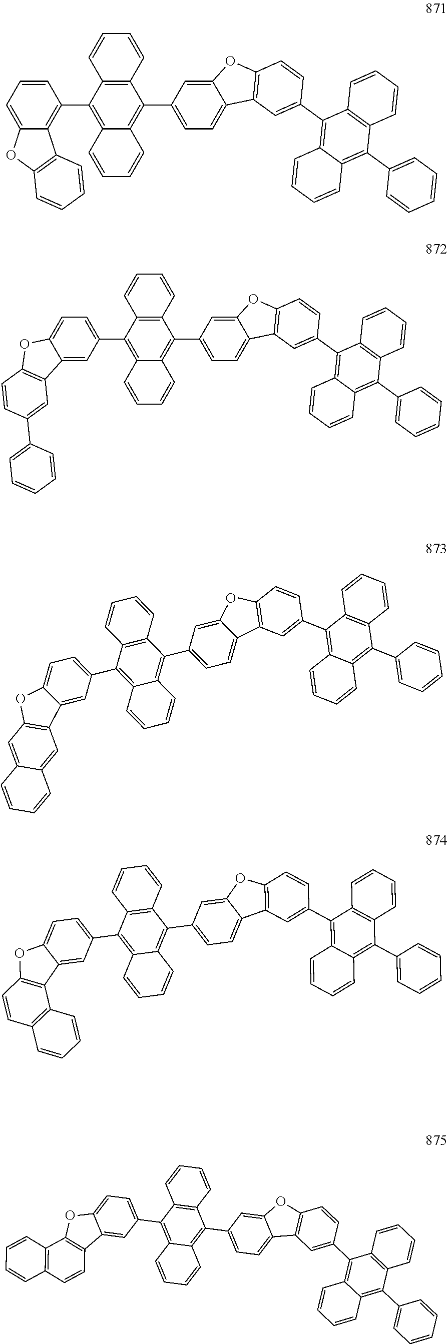

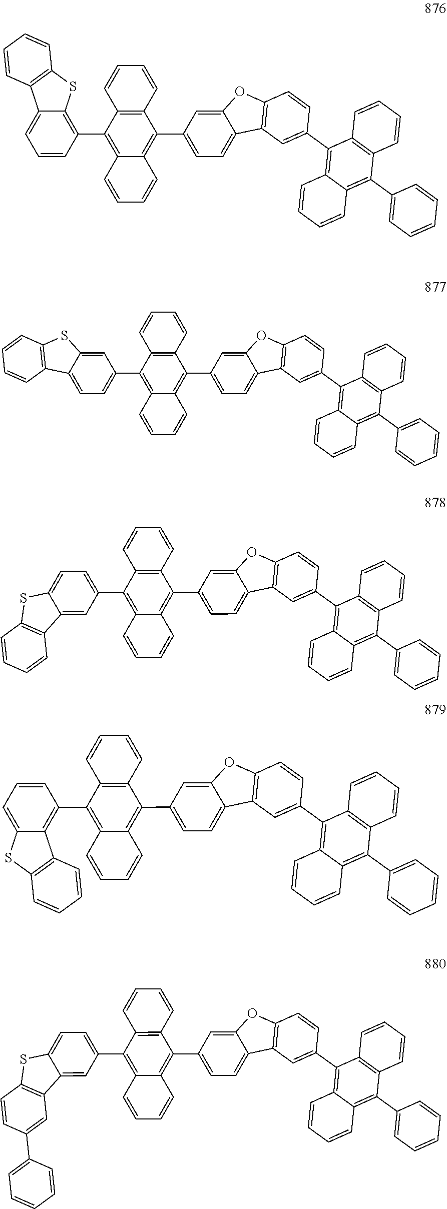

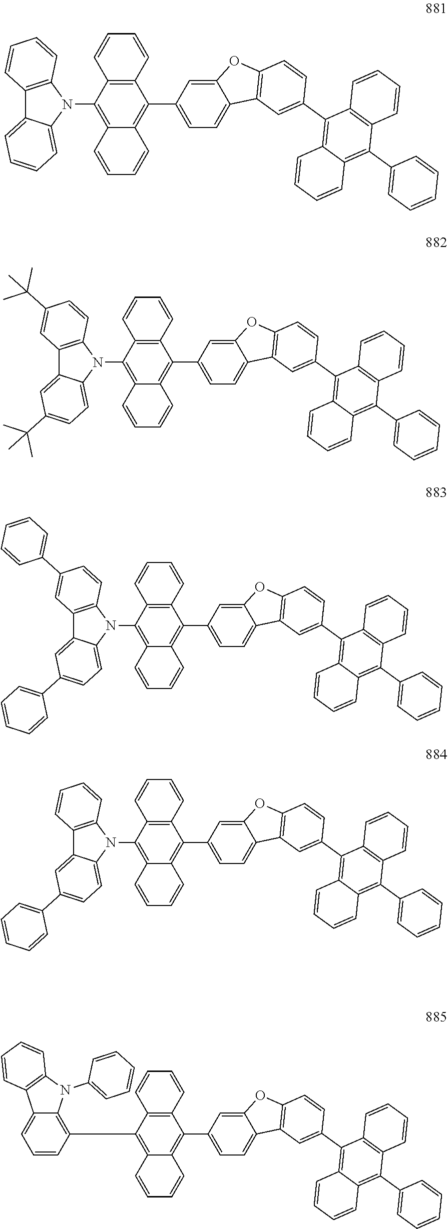

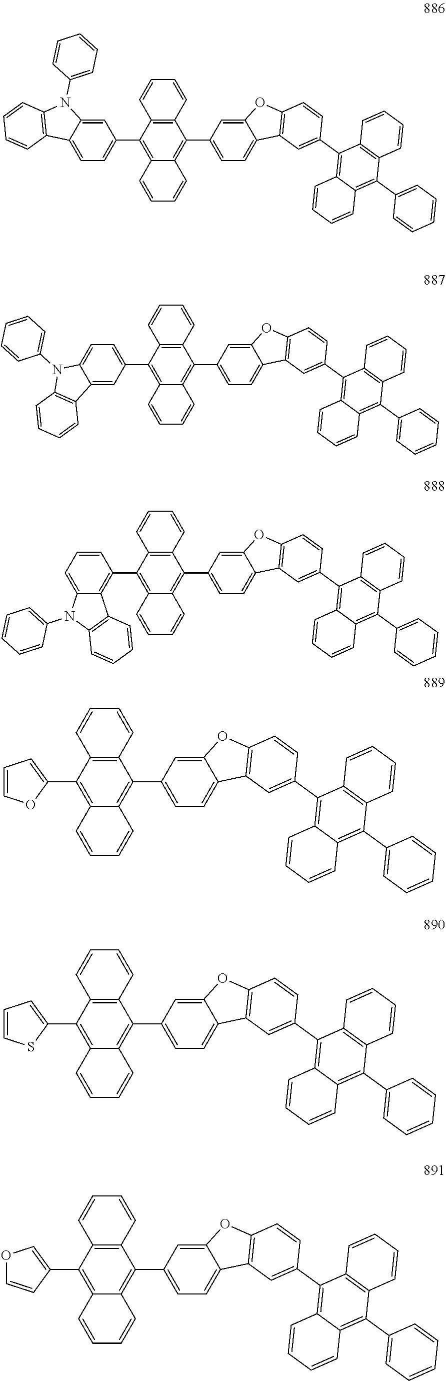

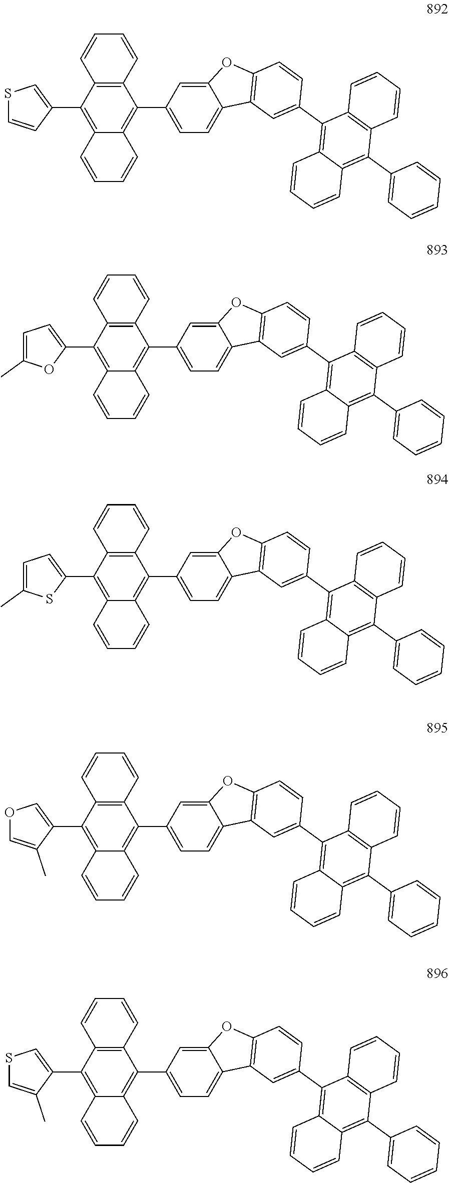 [SMILES: c1ccc(-c2c3ccccc3c(-c3ccc4oc5cc(-c6c7ccccc7c(-c7ccsc7)c7ccccc67)ccc5c4c3)c3ccccc23)cc1 ; Cc1ccc(-c2c3ccccc3c(-c3ccc4c(c3)oc3ccc(-c5c6ccccc6c(-c6ccccc6)c6ccccc56)cc34)c3ccccc23)o1 ; Cc1ccc(-c2c3ccccc3c(-c3ccc4c(c3)oc3ccc(-c5c6ccccc6c(-c6ccccc6)c6ccccc56)cc34)c3ccccc23)s1 ; Cc1cocc1-c1c2ccccc2c(-c2ccc3c(c2)oc2ccc(-c4c5ccccc5c(-c5ccccc5)c5ccccc45)cc23)c2ccccc12 ; Cc1cscc1-c1c2ccccc2c(-c2ccc3c(c2)oc2ccc(-c4c5ccccc5c(-c5ccccc5)c5ccccc45)cc23)c2ccccc12]

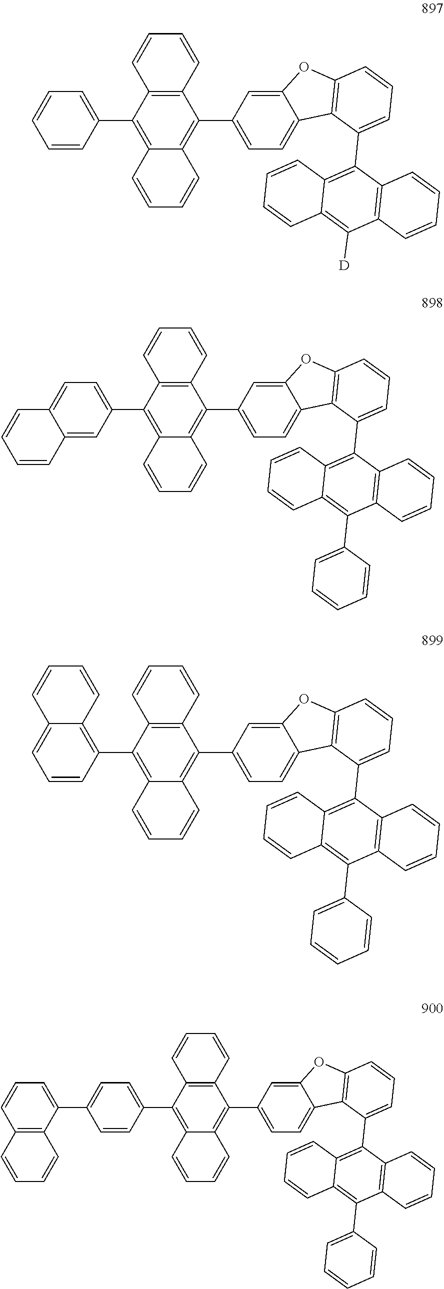 [SMILES: [2H]c1c2ccccc2c(-c2cccc3oc4cc(-c5c6ccccc6c(-c6ccccc6)c6ccccc56)ccc4c23)c2ccccc12 ; c1ccc(-c2c3ccccc3c(-c3cccc4oc5cc(-c6c7ccccc7c(-c7ccc8ccccc8c7)c7ccccc67)ccc5c34)c3ccccc23)cc1 ; c1ccc(-c2c3ccccc3c(-c3cccc4oc5cc(-c6c7ccccc7c(-c7cccc8ccccc78)c7ccccc67)ccc5c34)c3ccccc23)cc1 ; c1ccc(-c2c3ccccc3c(-c3cccc4oc5cc(-c6c7ccccc7c(-c7ccc(-c8cccc9ccccc89)cc7)c7ccccc67)ccc5c34)c3ccccc23)cc1]

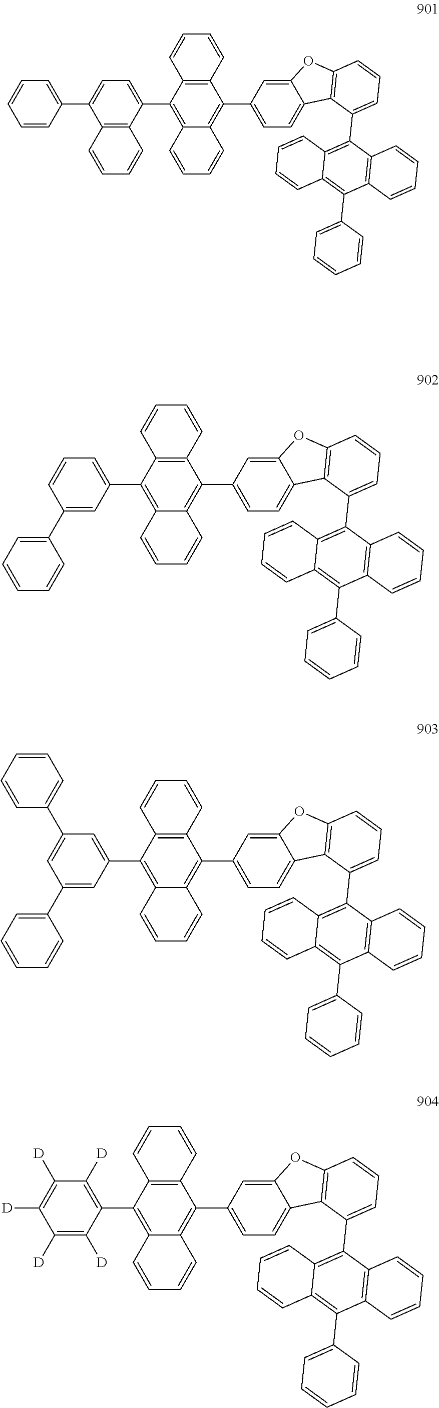 [SMILES: c1ccc(-c2ccc(-c3c4ccccc4c(-c4ccc5c(c4)oc4cccc(-c6c7ccccc7c(-c7ccccc7)c7ccccc67)c45)c4ccccc34)c3ccccc23)cc1 ; c1ccc(-c2cccc(-c3c4ccccc4c(-c4ccc5c(c4)oc4cccc(-c6c7ccccc7c(-c7ccccc7)c7ccccc67)c45)c4ccccc34)c2)cc1 ; c1ccc(-c2cc(-c3ccccc3)cc(-c3c4ccccc4c(-c4ccc5c(c4)oc4cccc(-c6c7ccccc7c(-c7ccccc7)c7ccccc67)c45)c4ccccc34)c2)cc1 ; [2H]c1c([2H])c([2H])c(-c2c3ccccc3c(-c3ccc4c(c3)oc3cccc(-c5c6ccccc6c(-c6ccccc6)c6ccccc56)c34)c3ccccc23)c([2H])c1[2H]]

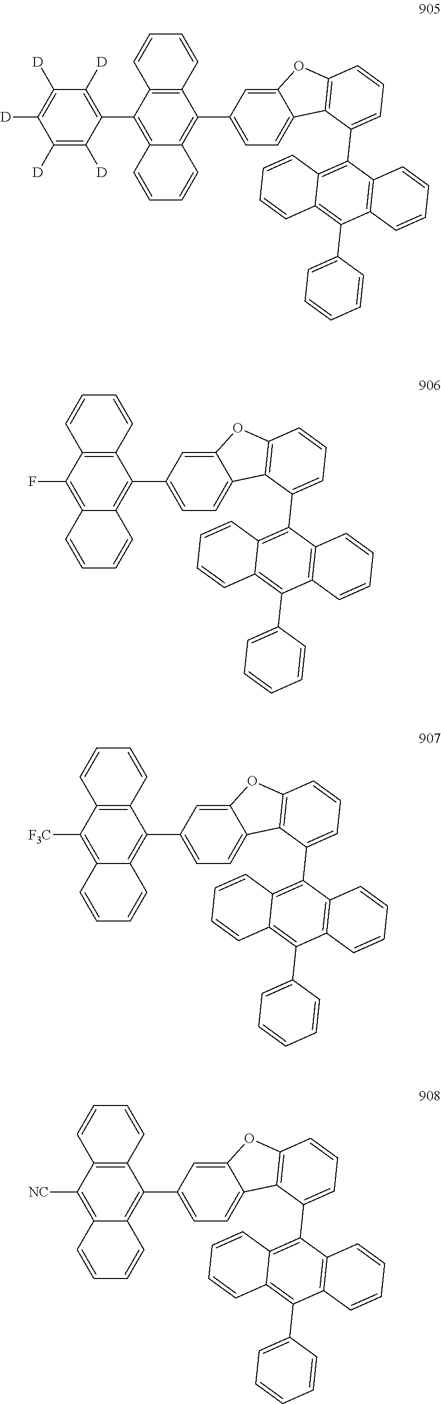 [SMILES: [2H]c1c([2H])c([2H])c(-c2c3ccccc3c(-c3ccc4c(c3)oc3cccc(-c5c6ccccc6c(-c6ccccc6)c6ccccc56)c34)c3ccccc23)c([2H])c1[2H] ; Fc1c2ccccc2c(-c2ccc3c(c2)oc2cccc(-c4c5ccccc5c(-c5ccccc5)c5ccccc45)c23)c2ccccc12 ; FC(F)(F)c1c2ccccc2c(-c2ccc3c(c2)oc2cccc(-c4c5ccccc5c(-c5ccccc5)c5ccccc45)c23)c2ccccc12 ; N#Cc1c2ccccc2c(-c2ccc3c(c2)oc2cccc(-c4c5ccccc5c(-c5ccccc5)c5ccccc45)c23)c2ccccc12]

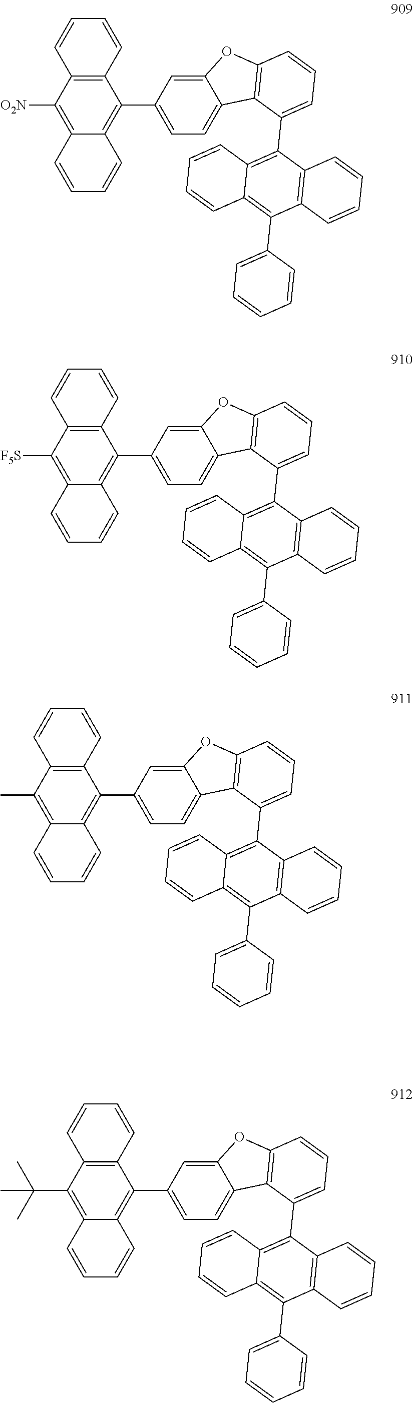

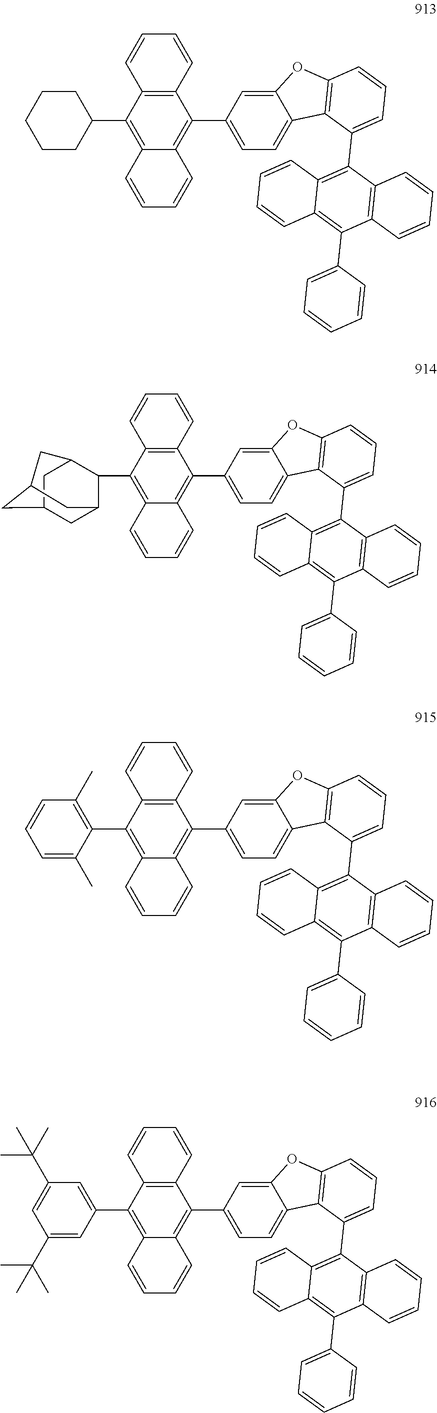 [SMILES: c1ccc(-c2c3ccccc3c(-c3cccc4oc5cc(-c6c7ccccc7c(C7CCCCC7)c7ccccc67)ccc5c34)c3ccccc23)cc1 ; c1ccc(-c2c3ccccc3c(-c3cccc4oc5cc(-c6c7ccccc7c(C7C8CC9CC(C8)CC7C9)c7ccccc67)ccc5c34)c3ccccc23)cc1 ; Cc1cccc(C)c1-c1c2ccccc2c(-c2ccc3c(c2)oc2cccc(-c4c5ccccc5c(-c5ccccc5)c5ccccc45)c23)c2ccccc12 ; CC(C)(C)c1cc(-c2c3ccccc3c(-c3ccc4c(c3)oc3cccc(-c5c6ccccc6c(-c6ccccc6)c6ccccc56)c34)c3ccccc23)cc(C(C)(C)C)c1]

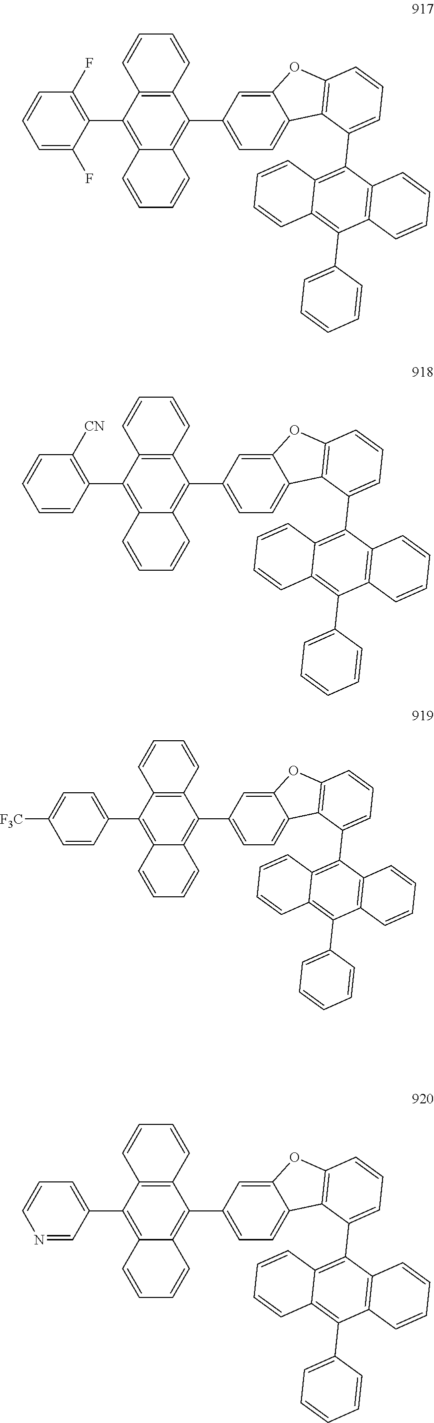 [SMILES: Fc1cccc(F)c1-c1c2ccccc2c(-c2ccc3c(c2)oc2cccc(-c4c5ccccc5c(-c5ccccc5)c5ccccc45)c23)c2ccccc12 ; N#Cc1ccccc1-c1c2ccccc2c(-c2ccc3c(c2)oc2cccc(-c4c5ccccc5c(-c5ccccc5)c5ccccc45)c23)c2ccccc12 ; FC(F)(F)c1ccc(-c2c3ccccc3c(-c3ccc4c(c3)oc3cccc(-c5c6ccccc6c(-c6ccccc6)c6ccccc56)c34)c3ccccc23)cc1 ; c1ccc(-c2c3ccccc3c(-c3cccc4oc5cc(-c6c7ccccc7c(-c7cccnc7)c7ccccc67)ccc5c34)c3ccccc23)cc1]

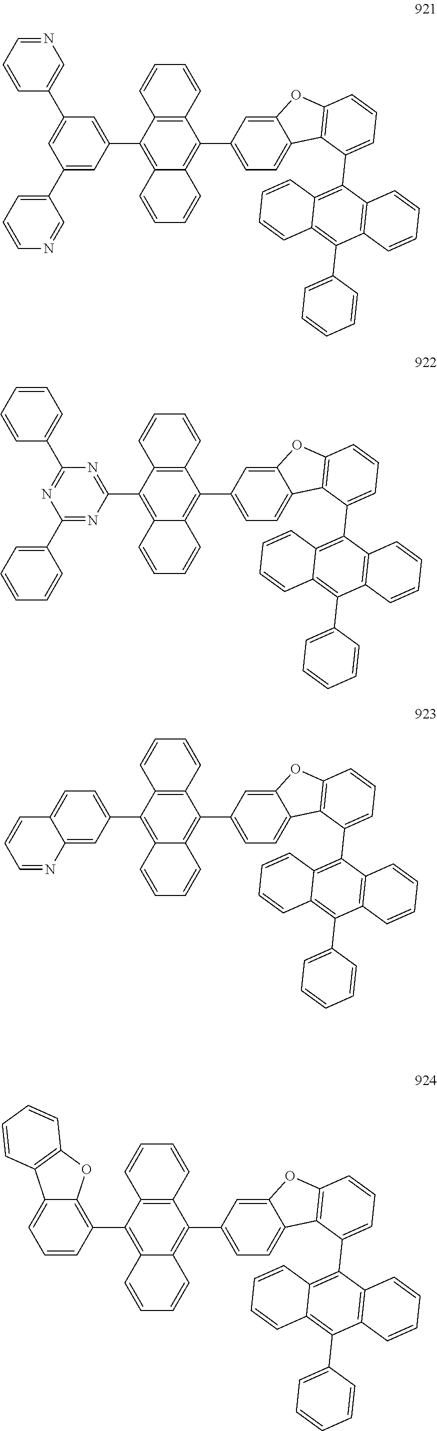 [SMILES: c1ccc(-c2c3ccccc3c(-c3cccc4oc5cc(-c6c7ccccc7c(-c7cc(-c8cccnc8)cc(-c8cccnc8)c7)c7ccccc67)ccc5c34)c3ccccc23)cc1 ; c1ccc(-c2nc(-c3ccccc3)nc(-c3c4ccccc4c(-c4ccc5c(c4)oc4cccc(-c6c7ccccc7c(-c7ccccc7)c7ccccc67)c45)c4ccccc34)n2)cc1 ; c1ccc(-c2c3ccccc3c(-c3cccc4oc5cc(-c6c7ccccc7c(-c7ccc8cccnc8c7)c7ccccc67)ccc5c34)c3ccccc23)cc1 ; c1ccc(-c2c3ccccc3c(-c3cccc4oc5cc(-c6c7ccccc7c(-c7cccc8c7oc7ccccc78)c7ccccc67)ccc5c34)c3ccccc23)cc1]

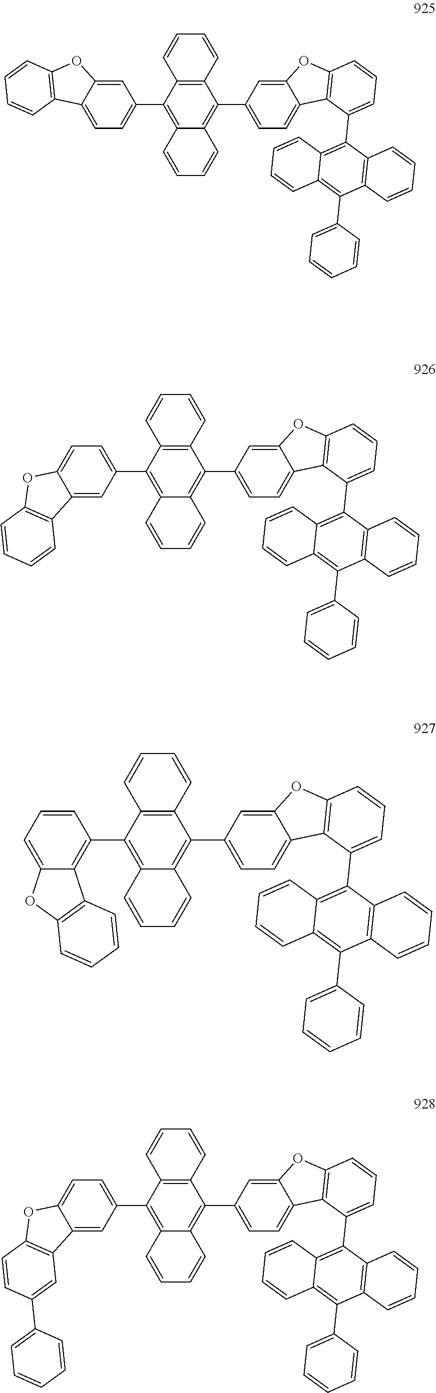 [SMILES: c1ccc(-c2c3ccccc3c(-c3cccc4oc5cc(-c6c7ccccc7c(-c7ccc8c(c7)oc7ccccc78)c7ccccc67)ccc5c34)c3ccccc23)cc1 ; c1ccc(-c2c3ccccc3c(-c3cccc4oc5cc(-c6c7ccccc7c(-c7ccc8oc9ccccc9c8c7)c7ccccc67)ccc5c34)c3ccccc23)cc1 ; c1ccc(-c2c3ccccc3c(-c3cccc4oc5cc(-c6c7ccccc7c(-c7cccc8oc9ccccc9c78)c7ccccc67)ccc5c34)c3ccccc23)cc1 ; c1ccc(-c2ccc3oc4ccc(-c5c6ccccc6c(-c6ccc7c(c6)oc6cccc(-c8c9ccccc9c(-c9ccccc9)c9ccccc89)c67)c6ccccc56)cc4c3c2)cc1]

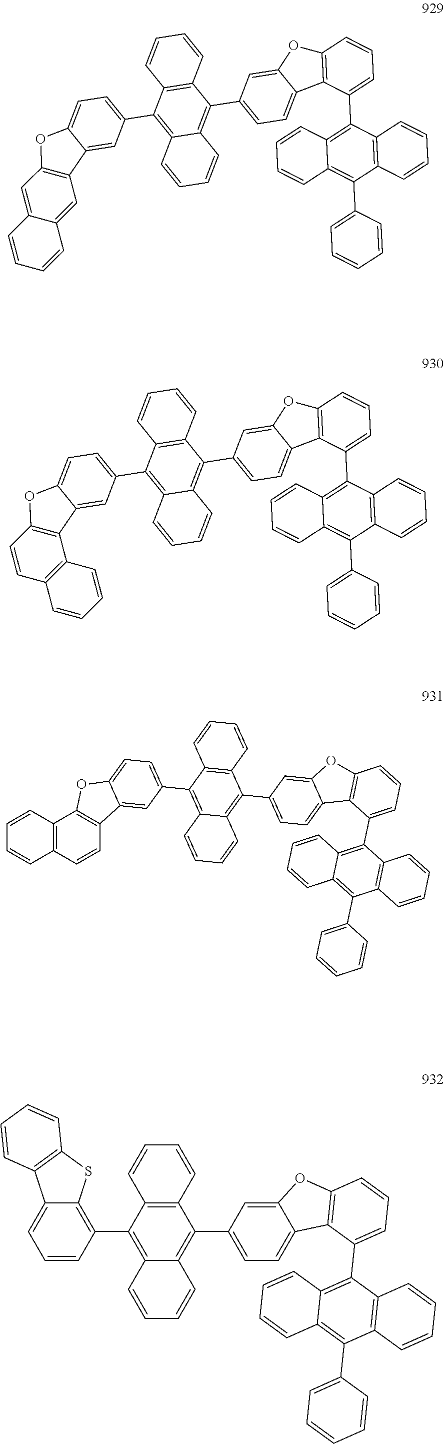 [SMILES: c1ccc(-c2c3ccccc3c(-c3cccc4oc5cc(-c6c7ccccc7c(-c7ccc8oc9cc%10ccccc%10cc9c8c7)c7ccccc67)ccc5c34)c3ccccc23)cc1 ; c1ccc(-c2c3ccccc3c(-c3cccc4oc5cc(-c6c7ccccc7c(-c7ccc8oc9ccc%10ccccc%10c9c8c7)c7ccccc67)ccc5c34)c3ccccc23)cc1 ; c1ccc(-c2c3ccccc3c(-c3cccc4oc5cc(-c6c7ccccc7c(-c7ccc8oc9c%10ccccc%10ccc9c8c7)c7ccccc67)ccc5c34)c3ccccc23)cc1 ; c1ccc(-c2c3ccccc3c(-c3cccc4oc5cc(-c6c7ccccc7c(-c7cccc8c7sc7ccccc78)c7ccccc67)ccc5c34)c3ccccc23)cc1]

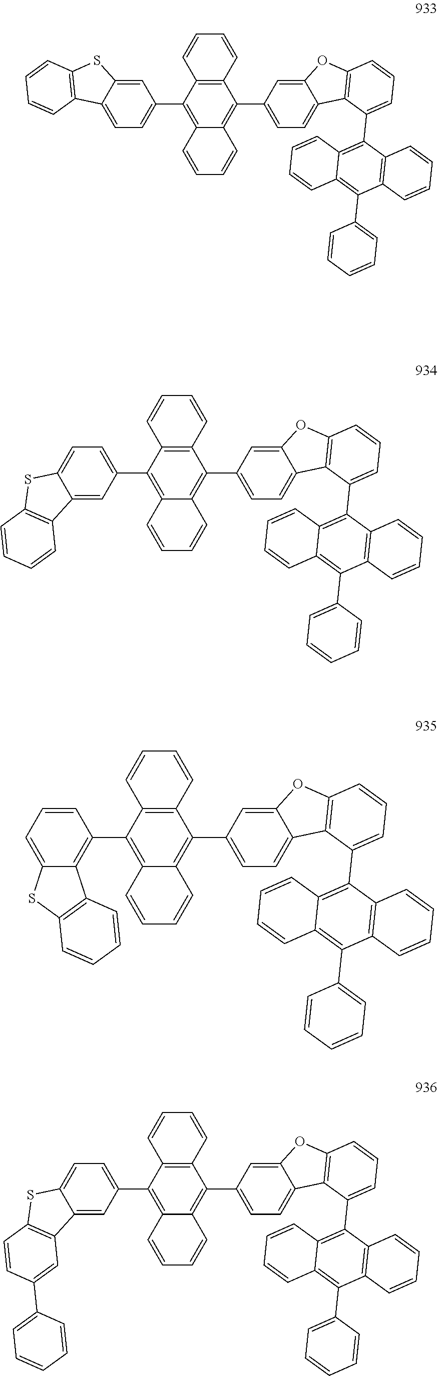 [SMILES: c1ccc(-c2c3ccccc3c(-c3cccc4oc5cc(-c6c7ccccc7c(-c7ccc8c(c7)sc7ccccc78)c7ccccc67)ccc5c34)c3ccccc23)cc1 ; c1ccc(-c2c3ccccc3c(-c3cccc4oc5cc(-c6c7ccccc7c(-c7ccc8sc9ccccc9c8c7)c7ccccc67)ccc5c34)c3ccccc23)cc1 ; c1ccc(-c2c3ccccc3c(-c3cccc4oc5cc(-c6c7ccccc7c(-c7cccc8sc9ccccc9c78)c7ccccc67)ccc5c34)c3ccccc23)cc1 ; c1ccc(-c2ccc3sc4ccc(-c5c6ccccc6c(-c6ccc7c(c6)oc6cccc(-c8c9ccccc9c(-c9ccccc9)c9ccccc89)c67)c6ccccc56)cc4c3c2)cc1]

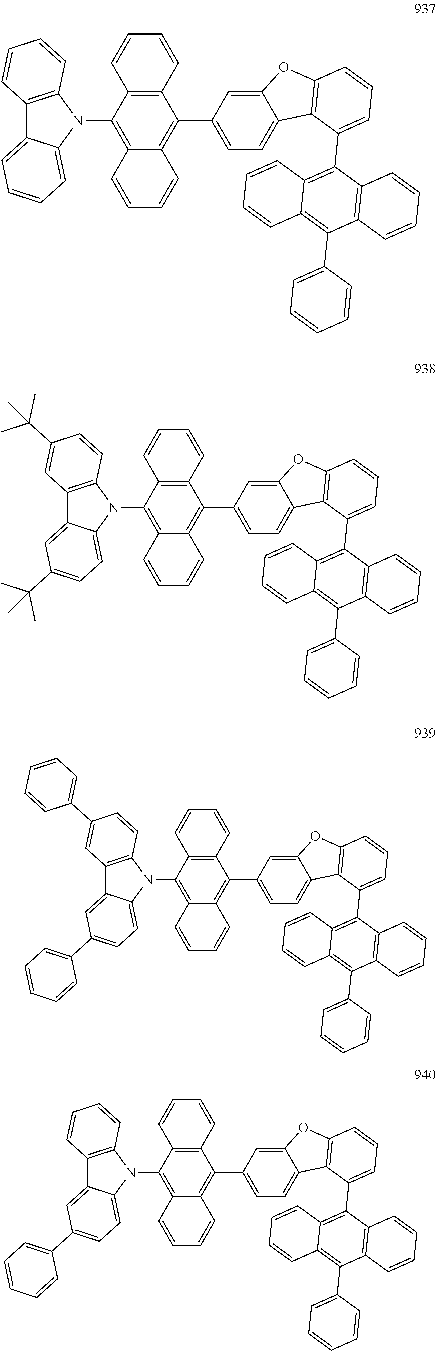 [SMILES: c1ccc(-c2c3ccccc3c(-c3cccc4oc5cc(-c6c7ccccc7c(-n7c8ccccc8c8ccccc87)c7ccccc67)ccc5c34)c3ccccc23)cc1 ; CC(C)(C)c1ccc2c(c1)c1cc(C(C)(C)C)ccc1n2-c1c2ccccc2c(-c2ccc3c(c2)oc2cccc(-c4c5ccccc5c(-c5ccccc5)c5ccccc45)c23)c2ccccc12 ; c1ccc(-c2ccc3c(c2)c2cc(-c4ccccc4)ccc2n3-c2c3ccccc3c(-c3ccc4c(c3)oc3cccc(-c5c6ccccc6c(-c6ccccc6)c6ccccc56)c34)c3ccccc23)cc1 ; c1ccc(-c2ccc3c(c2)c2ccccc2n3-c2c3ccccc3c(-c3ccc4c(c3)oc3cccc(-c5c6ccccc6c(-c6ccccc6)c6ccccc56)c34)c3ccccc23)cc1]

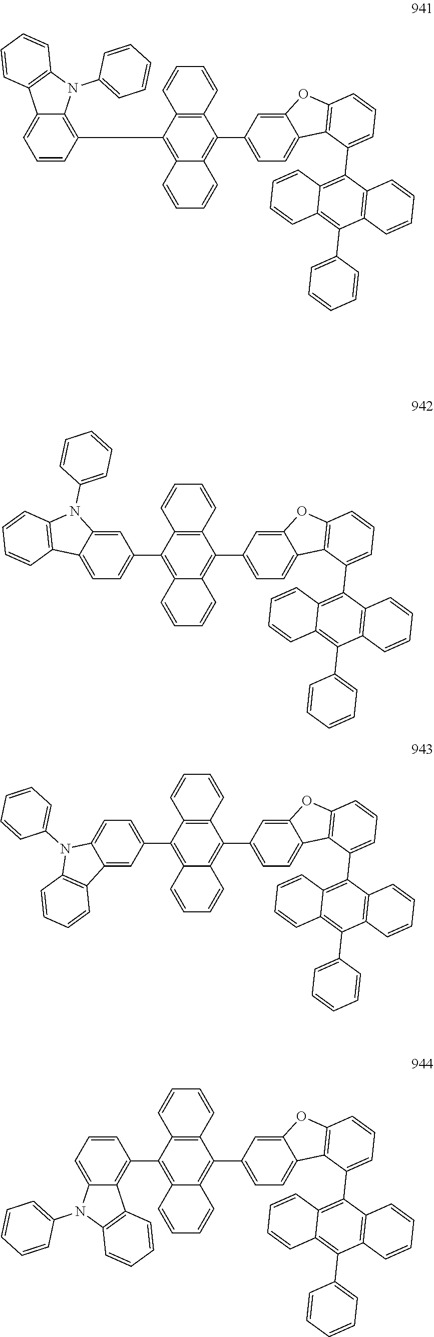 [SMILES: c1ccc(-c2c3ccccc3c(-c3cccc4oc5cc(-c6c7ccccc7c(-c7cccc8c9ccccc9n(-c9ccccc9)c78)c7ccccc67)ccc5c34)c3ccccc23)cc1 ; c1ccc(-c2c3ccccc3c(-c3cccc4oc5cc(-c6c7ccccc7c(-c7ccc8c9ccccc9n(-c9ccccc9)c8c7)c7ccccc67)ccc5c34)c3ccccc23)cc1 ; c1ccc(-c2c3ccccc3c(-c3cccc4oc5cc(-c6c7ccccc7c(-c7ccc8c(c7)c7ccccc7n8-c7ccccc7)c7ccccc67)ccc5c34)c3ccccc23)cc1 ; c1ccc(-c2c3ccccc3c(-c3cccc4oc5cc(-c6c7ccccc7c(-c7cccc8c7c7ccccc7n8-c7ccccc7)c7ccccc67)ccc5c34)c3ccccc23)cc1]

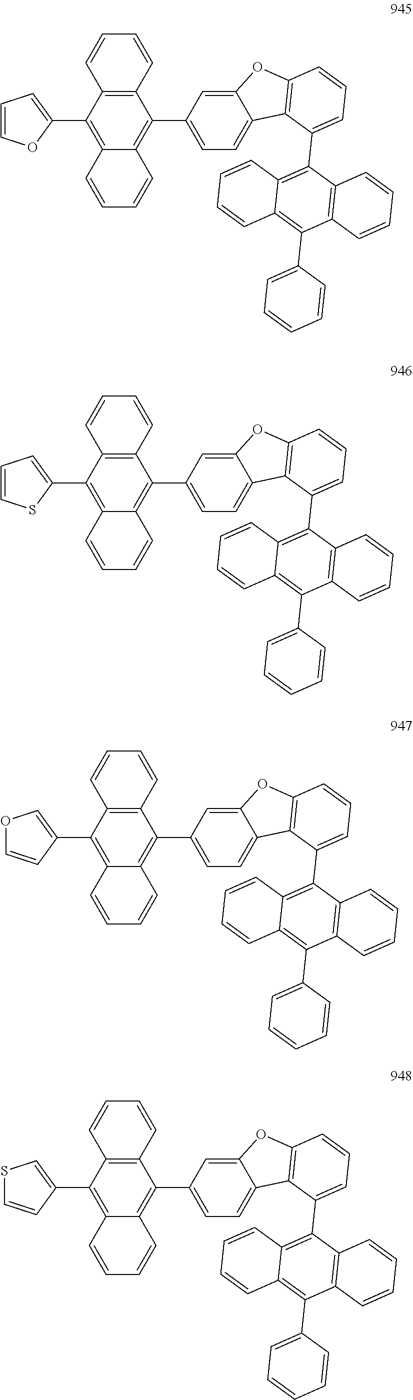 [SMILES: c1ccc(-c2c3ccccc3c(-c3cccc4oc5cc(-c6c7ccccc7c(-c7ccco7)c7ccccc67)ccc5c34)c3ccccc23)cc1 ; c1ccc(-c2c3ccccc3c(-c3cccc4oc5cc(-c6c7ccccc7c(-c7cccs7)c7ccccc67)ccc5c34)c3ccccc23)cc1 ; c1ccc(-c2c3ccccc3c(-c3cccc4oc5cc(-c6c7ccccc7c(-c7ccoc7)c7ccccc67)ccc5c34)c3ccccc23)cc1 ; c1ccc(-c2c3ccccc3c(-c3cccc4oc5cc(-c6c7ccccc7c(-c7ccsc7)c7ccccc67)ccc5c34)c3ccccc23)cc1]

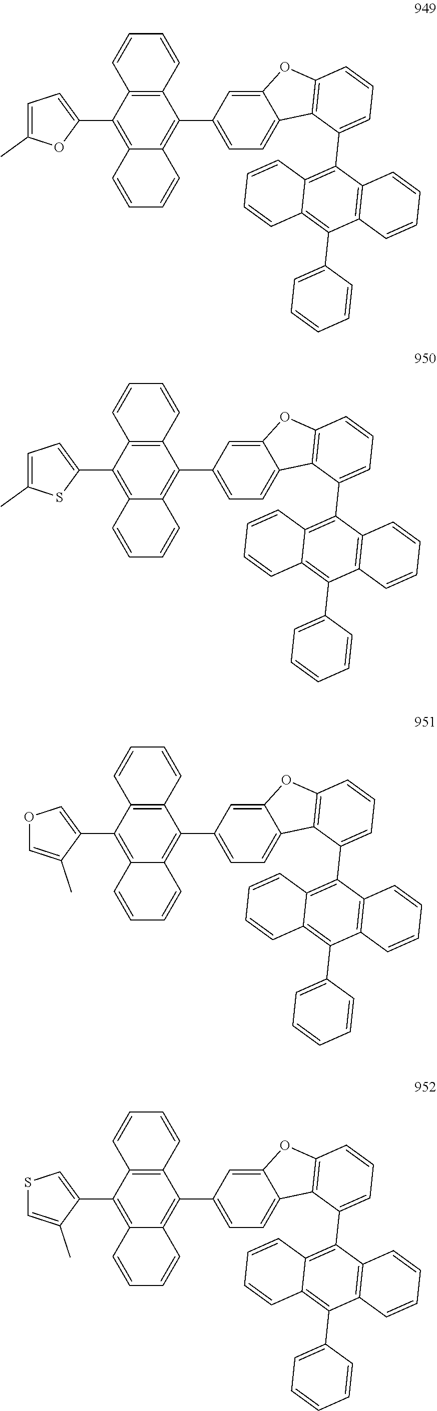 [SMILES: Cc1ccc(-c2c3ccccc3c(-c3ccc4c(c3)oc3cccc(-c5c6ccccc6c(-c6ccccc6)c6ccccc56)c34)c3ccccc23)o1 ; Cc1ccc(-c2c3ccccc3c(-c3ccc4c(c3)oc3cccc(-c5c6ccccc6c(-c6ccccc6)c6ccccc56)c34)c3ccccc23)s1 ; Cc1cocc1-c1c2ccccc2c(-c2ccc3c(c2)oc2cccc(-c4c5ccccc5c(-c5ccccc5)c5ccccc45)c23)c2ccccc12 ; Cc1cscc1-c1c2ccccc2c(-c2ccc3c(c2)oc2cccc(-c4c5ccccc5c(-c5ccccc5)c5ccccc45)c23)c2ccccc12]

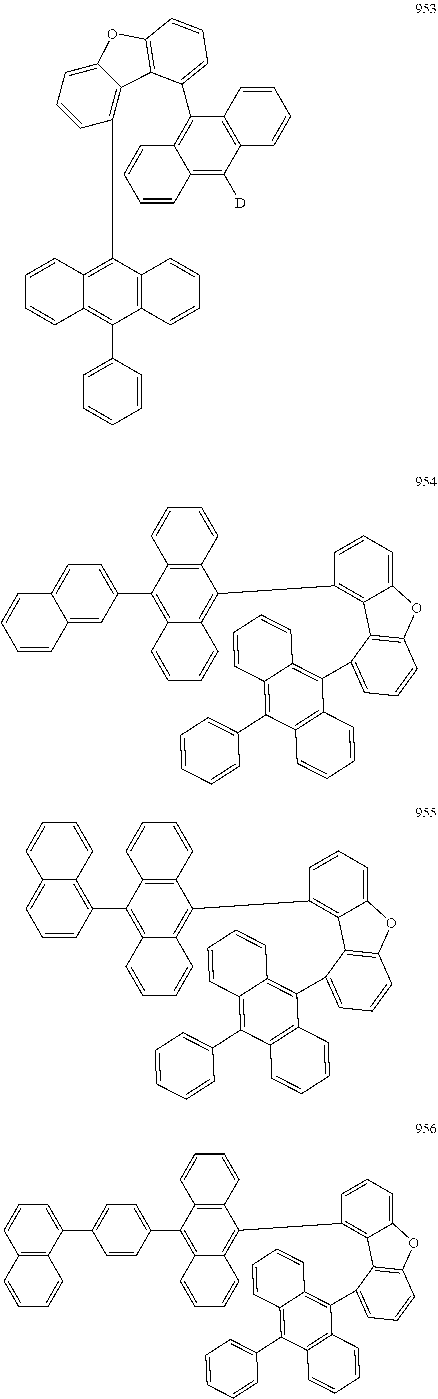 [SMILES: [2H]c1c2ccccc2c(-c2cccc3oc4cccc(-c5c6ccccc6c(-c6ccccc6)c6ccccc56)c4c23)c2ccccc12 ; c1ccc(-c2c3ccccc3c(-c3cccc4oc5cccc(-c6c7ccccc7c(-c7ccc8ccccc8c7)c7ccccc67)c5c34)c3ccccc23)cc1 ; c1ccc(-c2c3ccccc3c(-c3cccc4oc5cccc(-c6c7ccccc7c(-c7cccc8ccccc78)c7ccccc67)c5c34)c3ccccc23)cc1 ; c1ccc(-c2c3ccccc3c(-c3cccc4oc5cccc(-c6c7ccccc7c(-c7ccc(-c8cccc9ccccc89)cc7)c7ccccc67)c5c34)c3ccccc23)cc1]

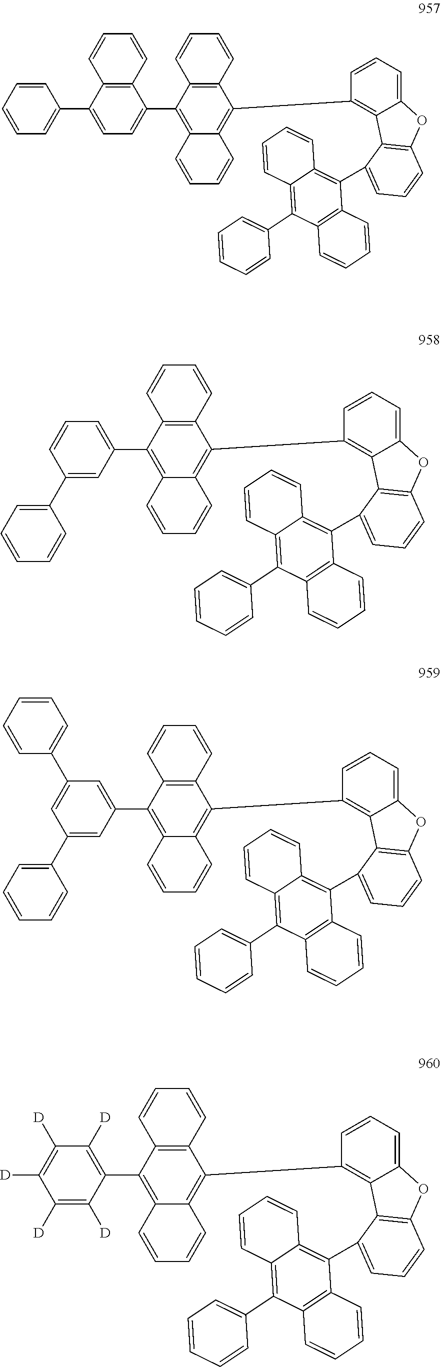 [SMILES: c1ccc(-c2ccc(-c3c4ccccc4c(-c4cccc5oc6cccc(-c7c8ccccc8c(-c8ccccc8)c8ccccc78)c6c45)c4ccccc34)c3ccccc23)cc1 ; c1ccc(-c2cccc(-c3c4ccccc4c(-c4cccc5oc6cccc(-c7c8ccccc8c(-c8ccccc8)c8ccccc78)c6c45)c4ccccc34)c2)cc1 ; c1ccc(-c2cc(-c3ccccc3)cc(-c3c4ccccc4c(-c4cccc5oc6cccc(-c7c8ccccc8c(-c8ccccc8)c8ccccc78)c6c45)c4ccccc34)c2)cc1 ; [2H]c1c([2H])c([2H])c(-c2c3ccccc3c(-c3cccc4oc5cccc(-c6c7ccccc7c(-c7ccccc7)c7ccccc67)c5c34)c3ccccc23)c([2H])c1[2H]]

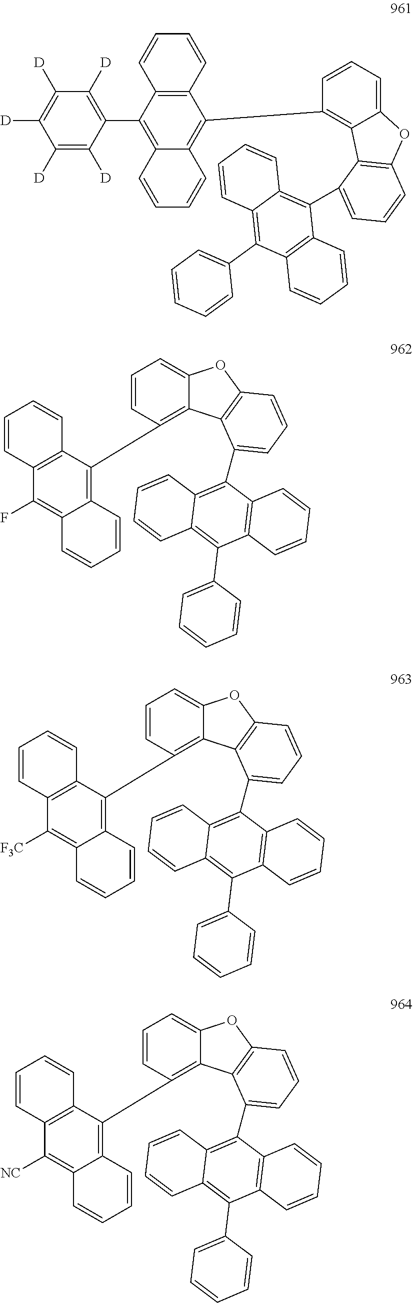

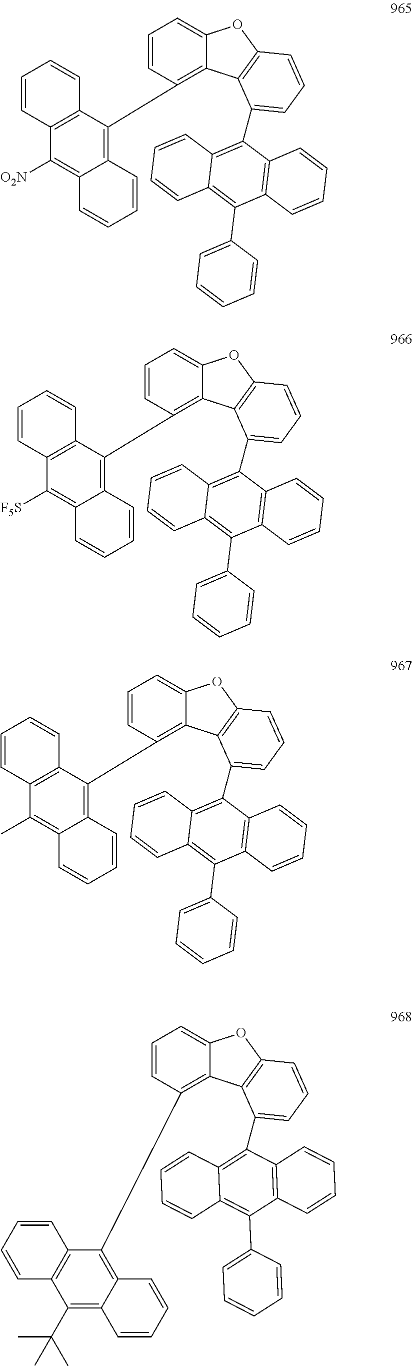

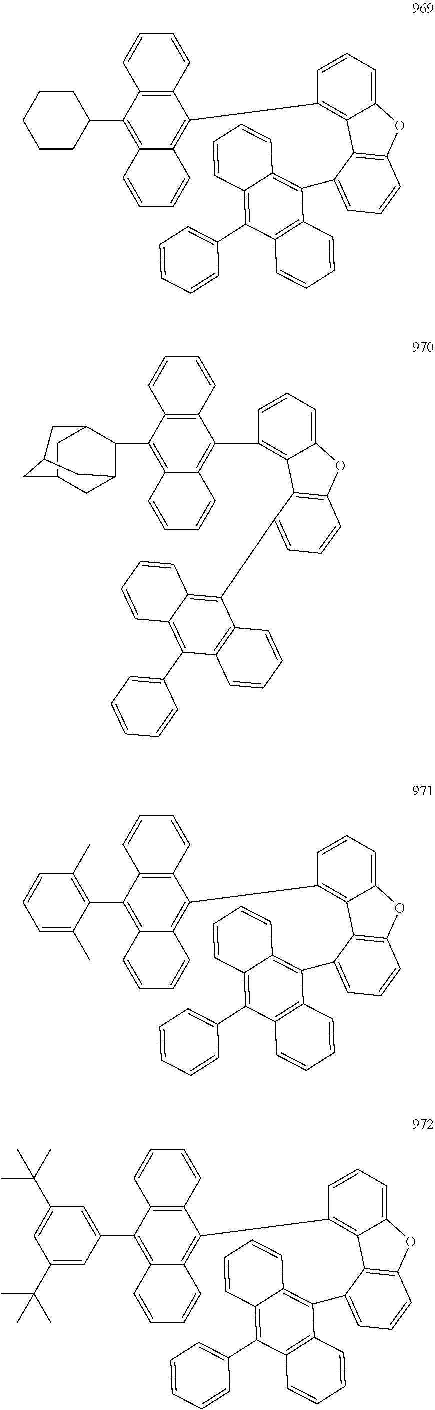 [SMILES: c1ccc(-c2c3ccccc3c(-c3cccc4oc5cccc(-c6c7ccccc7c(C7CCCCC7)c7ccccc67)c5c34)c3ccccc23)cc1 ; c1ccc(-c2c3ccccc3c(-c3cccc4oc5cccc(-c6c7ccccc7c(C7C8CC9CC(C8)CC7C9)c7ccccc67)c5c34)c3ccccc23)cc1 ; Cc1cccc(C)c1-c1c2ccccc2c(-c2cccc3oc4cccc(-c5c6ccccc6c(-c6ccccc6)c6ccccc56)c4c23)c2ccccc12 ; CC(C)(C)c1cc(-c2c3ccccc3c(-c3cccc4oc5cccc(-c6c7ccccc7c(-c7ccccc7)c7ccccc67)c5c34)c3ccccc23)cc(C(C)(C)C)c1]

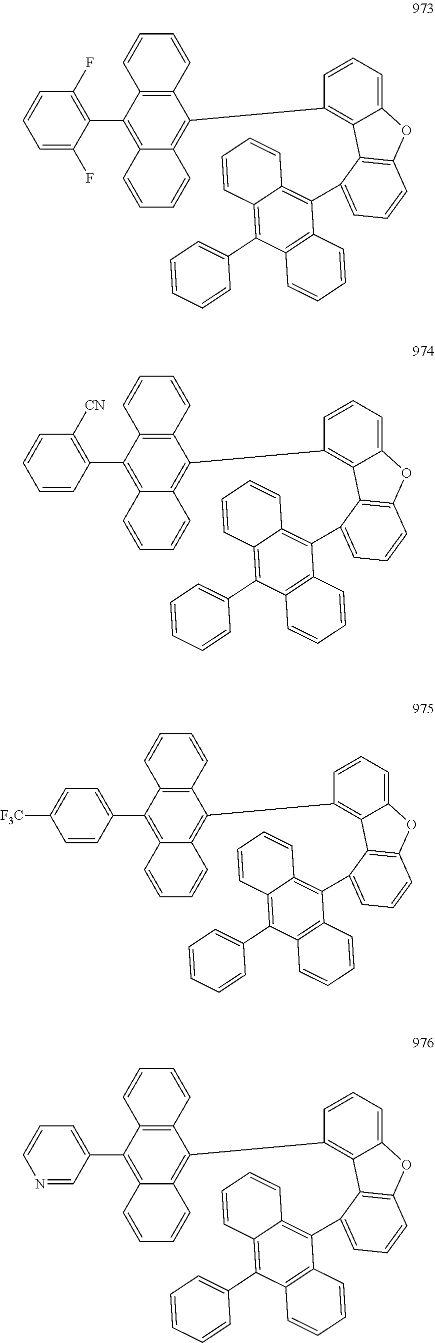 [SMILES: Fc1cccc(F)c1-c1c2ccccc2c(-c2cccc3oc4cccc(-c5c6ccccc6c(-c6ccccc6)c6ccccc56)c4c23)c2ccccc12 ; N#Cc1ccccc1-c1c2ccccc2c(-c2cccc3oc4cccc(-c5c6ccccc6c(-c6ccccc6)c6ccccc56)c4c23)c2ccccc12 ; FC(F)(F)c1ccc(-c2c3ccccc3c(-c3cccc4oc5cccc(-c6c7ccccc7c(-c7ccccc7)c7ccccc67)c5c34)c3ccccc23)cc1 ; c1ccc(-c2c3ccccc3c(-c3cccc4oc5cccc(-c6c7ccccc7c(-c7cccnc7)c7ccccc67)c5c34)c3ccccc23)cc1]

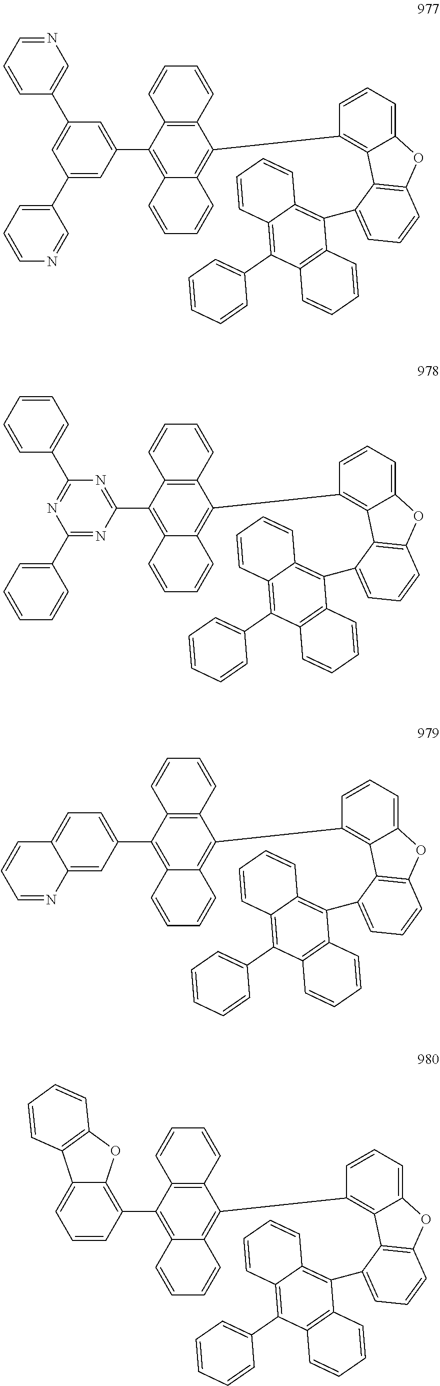 [SMILES: c1ccc(-c2c3ccccc3c(-c3cccc4oc5cccc(-c6c7ccccc7c(-c7cc(-c8cccnc8)cc(-c8cccnc8)c7)c7ccccc67)c5c34)c3ccccc23)cc1 ; c1ccc(-c2nc(-c3ccccc3)nc(-c3c4ccccc4c(-c4cccc5oc6cccc(-c7c8ccccc8c(-c8ccccc8)c8ccccc78)c6c45)c4ccccc34)n2)cc1 ; c1ccc(-c2c3ccccc3c(-c3cccc4oc5cccc(-c6c7ccccc7c(-c7ccc8cccnc8c7)c7ccccc67)c5c34)c3ccccc23)cc1 ; c1ccc(-c2c3ccccc3c(-c3cccc4oc5cccc(-c6c7ccccc7c(-c7cccc8c7oc7ccccc78)c7ccccc67)c5c34)c3ccccc23)cc1]

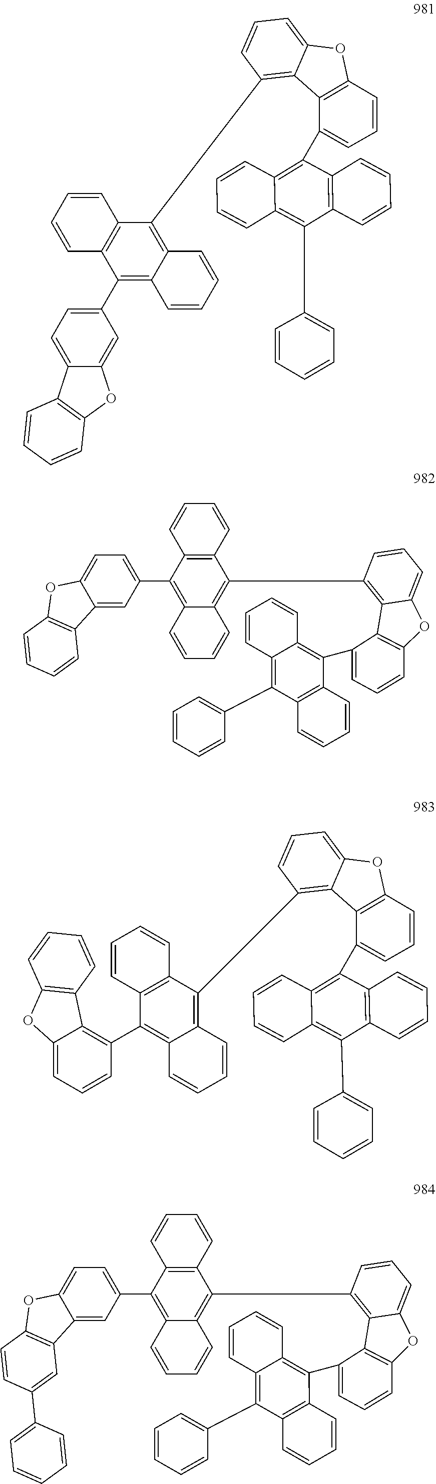 [SMILES: c1ccc(-c2c3ccccc3c(-c3cccc4oc5cccc(-c6c7ccccc7c(-c7ccc8c(c7)oc7ccccc78)c7ccccc67)c5c34)c3ccccc23)cc1 ; c1ccc(-c2c3ccccc3c(-c3cccc4oc5cccc(-c6c7ccccc7c(-c7ccc8oc9ccccc9c8c7)c7ccccc67)c5c34)c3ccccc23)cc1 ; c1ccc(-c2c3ccccc3c(-c3cccc4oc5cccc(-c6c7ccccc7c(-c7cccc8oc9ccccc9c78)c7ccccc67)c5c34)c3ccccc23)cc1 ; c1ccc(-c2ccc3oc4ccc(-c5c6ccccc6c(-c6cccc7oc8cccc(-c9c%10ccccc%10c(-c%10ccccc%10)c%10ccccc9%10)c8c67)c6ccccc56)cc4c3c2)cc1]

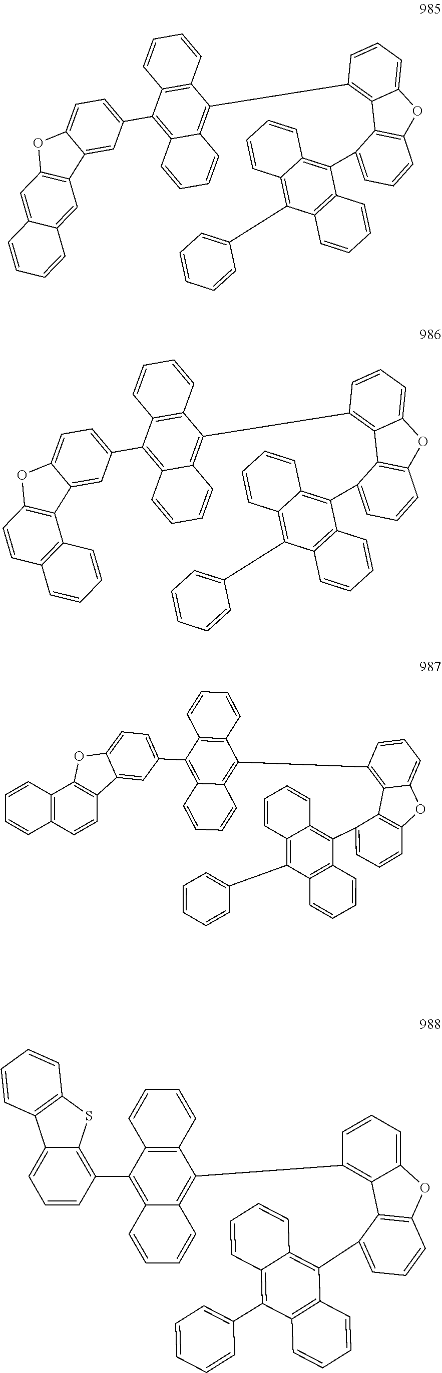 [SMILES: c1ccc(-c2c3ccccc3c(-c3cccc4oc5cccc(-c6c7ccccc7c(-c7ccc8oc9cc%10ccccc%10cc9c8c7)c7ccccc67)c5c34)c3ccccc23)cc1 ; c1ccc(-c2c3ccccc3c(-c3cccc4oc5cccc(-c6c7ccccc7c(-c7ccc8oc9ccc%10ccccc%10c9c8c7)c7ccccc67)c5c34)c3ccccc23)cc1 ; c1ccc(-c2c3ccccc3c(-c3cccc4oc5cccc(-c6c7ccccc7c(-c7ccc8oc9c%10ccccc%10ccc9c8c7)c7ccccc67)c5c34)c3ccccc23)cc1 ; c1ccc(-c2c3ccccc3c(-c3cccc4oc5cccc(-c6c7ccccc7c(-c7cccc8c7sc7ccccc78)c7ccccc67)c5c34)c3ccccc23)cc1]

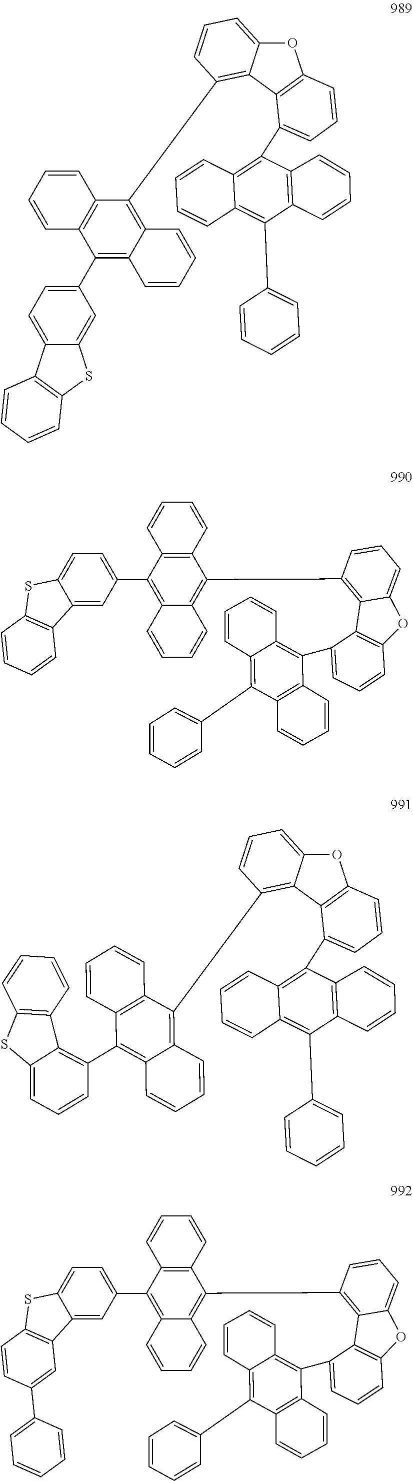 [SMILES: c1ccc(-c2c3ccccc3c(-c3cccc4oc5cccc(-c6c7ccccc7c(-c7ccc8c(c7)sc7ccccc78)c7ccccc67)c5c34)c3ccccc23)cc1 ; c1ccc(-c2c3ccccc3c(-c3cccc4oc5cccc(-c6c7ccccc7c(-c7ccc8sc9ccccc9c8c7)c7ccccc67)c5c34)c3ccccc23)cc1 ; c1ccc(-c2c3ccccc3c(-c3cccc4oc5cccc(-c6c7ccccc7c(-c7cccc8sc9ccccc9c78)c7ccccc67)c5c34)c3ccccc23)cc1 ; c1ccc(-c2ccc3sc4ccc(-c5c6ccccc6c(-c6cccc7oc8cccc(-c9c%10ccccc%10c(-c%10ccccc%10)c%10ccccc9%10)c8c67)c6ccccc56)cc4c3c2)cc1]

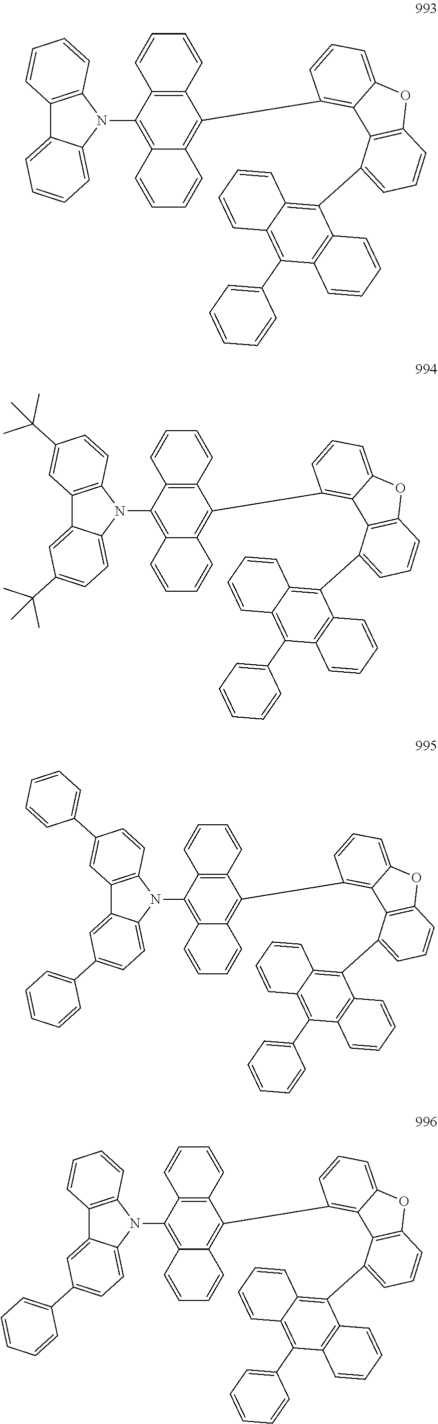 [SMILES: c1ccc(-c2c3ccccc3c(-c3cccc4oc5cccc(-c6c7ccccc7c(-n7c8ccccc8c8ccccc87)c7ccccc67)c5c34)c3ccccc23)cc1 ; CC(C)(C)c1ccc2c(c1)c1cc(C(C)(C)C)ccc1n2-c1c2ccccc2c(-c2cccc3oc4cccc(-c5c6ccccc6c(-c6ccccc6)c6ccccc56)c4c23)c2ccccc12 ; c1ccc(-c2ccc3c(c2)c2cc(-c4ccccc4)ccc2n3-c2c3ccccc3c(-c3cccc4oc5cccc(-c6c7ccccc7c(-c7ccccc7)c7ccccc67)c5c34)c3ccccc23)cc1 ; c1ccc(-c2ccc3c(c2)c2ccccc2n3-c2c3ccccc3c(-c3cccc4oc5cccc(-c6c7ccccc7c(-c7ccccc7)c7ccccc67)c5c34)c3ccccc23)cc1]

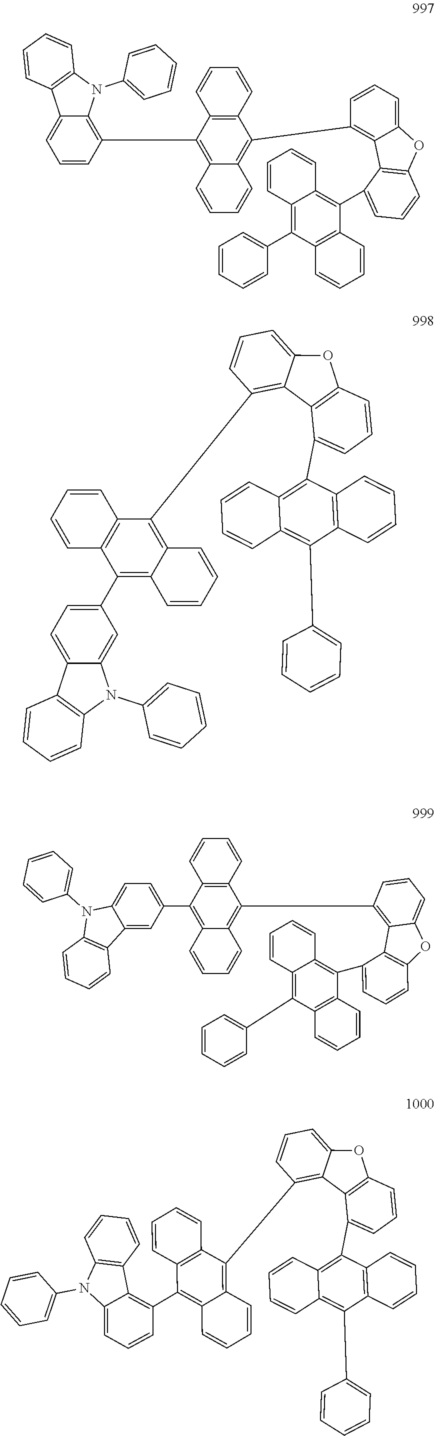 [SMILES: c1ccc(-c2c3ccccc3c(-c3cccc4oc5cccc(-c6c7ccccc7c(-c7cccc8c9ccccc9n(-c9ccccc9)c78)c7ccccc67)c5c34)c3ccccc23)cc1 ; c1ccc(-c2c3ccccc3c(-c3cccc4oc5cccc(-c6c7ccccc7c(-c7ccc8c9ccccc9n(-c9ccccc9)c8c7)c7ccccc67)c5c34)c3ccccc23)cc1 ; c1ccc(-c2c3ccccc3c(-c3cccc4oc5cccc(-c6c7ccccc7c(-c7ccc8c(c7)c7ccccc7n8-c7ccccc7)c7ccccc67)c5c34)c3ccccc23)cc1 ; c1ccc(-c2c3ccccc3c(-c3cccc4oc5cccc(-c6c7ccccc7c(-c7cccc8c7c7ccccc7n8-c7ccccc7)c7ccccc67)c5c34)c3ccccc23)cc1]

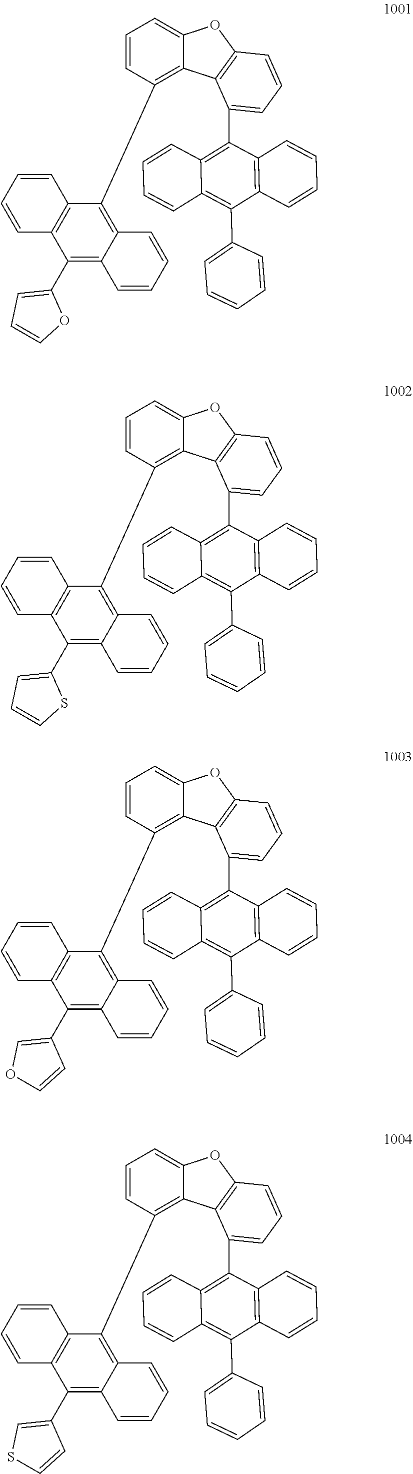 [SMILES: c1ccc(-c2c3ccccc3c(-c3cccc4oc5cccc(-c6c7ccccc7c(-c7ccco7)c7ccccc67)c5c34)c3ccccc23)cc1 ; c1ccc(-c2c3ccccc3c(-c3cccc4oc5cccc(-c6c7ccccc7c(-c7cccs7)c7ccccc67)c5c34)c3ccccc23)cc1 ; c1ccc(-c2c3ccccc3c(-c3cccc4oc5cccc(-c6c7ccccc7c(-c7ccoc7)c7ccccc67)c5c34)c3ccccc23)cc1 ; c1ccc(-c2c3ccccc3c(-c3cccc4oc5cccc(-c6c7ccccc7c(-c7ccsc7)c7ccccc67)c5c34)c3ccccc23)cc1]

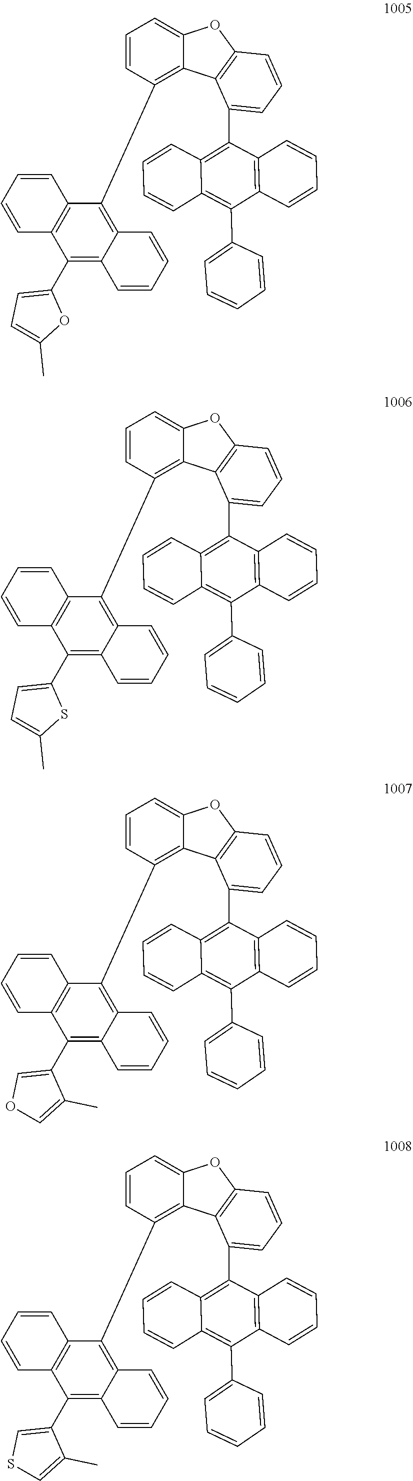 [SMILES: Cc1ccc(-c2c3ccccc3c(-c3cccc4oc5cccc(-c6c7ccccc7c(-c7ccccc7)c7ccccc67)c5c34)c3ccccc23)o1 ; Cc1ccc(-c2c3ccccc3c(-c3cccc4oc5cccc(-c6c7ccccc7c(-c7ccccc7)c7ccccc67)c5c34)c3ccccc23)s1 ; Cc1cocc1-c1c2ccccc2c(-c2cccc3oc4cccc(-c5c6ccccc6c(-c6ccccc6)c6ccccc56)c4c23)c2ccccc12 ; Cc1cscc1-c1c2ccccc2c(-c2cccc3oc4cccc(-c5c6ccccc6c(-c6ccccc6)c6ccccc56)c4c23)c2ccccc12]

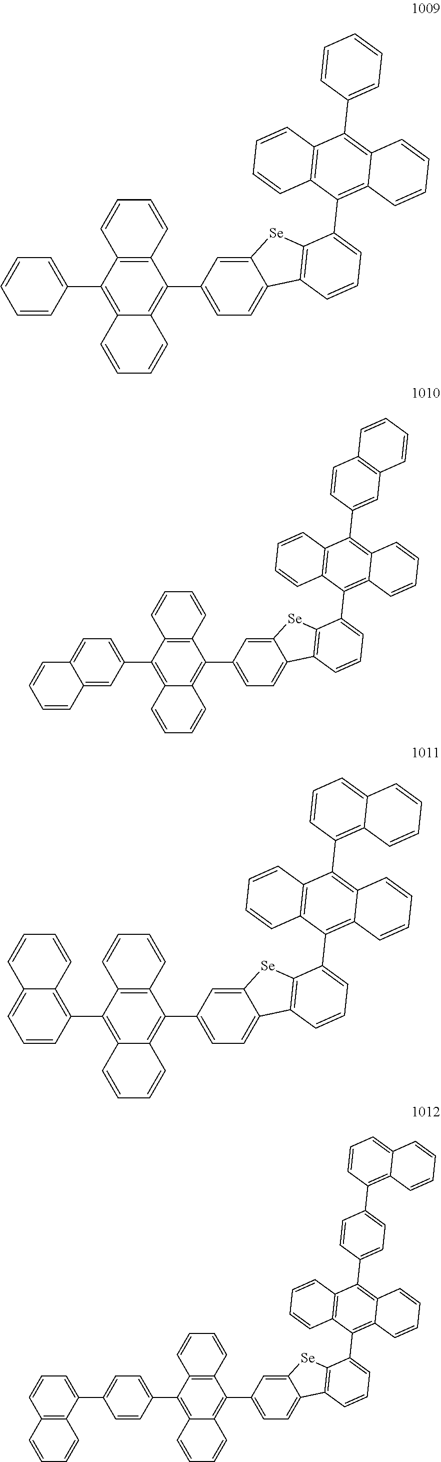 [SMILES: c1ccc(-c2c3ccccc3c(-c3ccc4c(c3)[se]c3c(-c5c6ccccc6c(-c6ccccc6)c6ccccc56)cccc34)c3ccccc23)cc1 ; c1ccc2cc(-c3c4ccccc4c(-c4ccc5c(c4)[se]c4c(-c6c7ccccc7c(-c7ccc8ccccc8c7)c7ccccc67)cccc45)c4ccccc34)ccc2c1 ; c1ccc2c(-c3c4ccccc4c(-c4ccc5c(c4)[se]c4c(-c6c7ccccc7c(-c7cccc8ccccc78)c7ccccc67)cccc45)c4ccccc34)cccc2c1 ; c1ccc2c(-c3ccc(-c4c5ccccc5c(-c5ccc6c(c5)[se]c5c(-c7c8ccccc8c(-c8ccc(-c9cccc%10ccccc9%10)cc8)c8ccccc78)cccc56)c5ccccc45)cc3)cccc2c1]

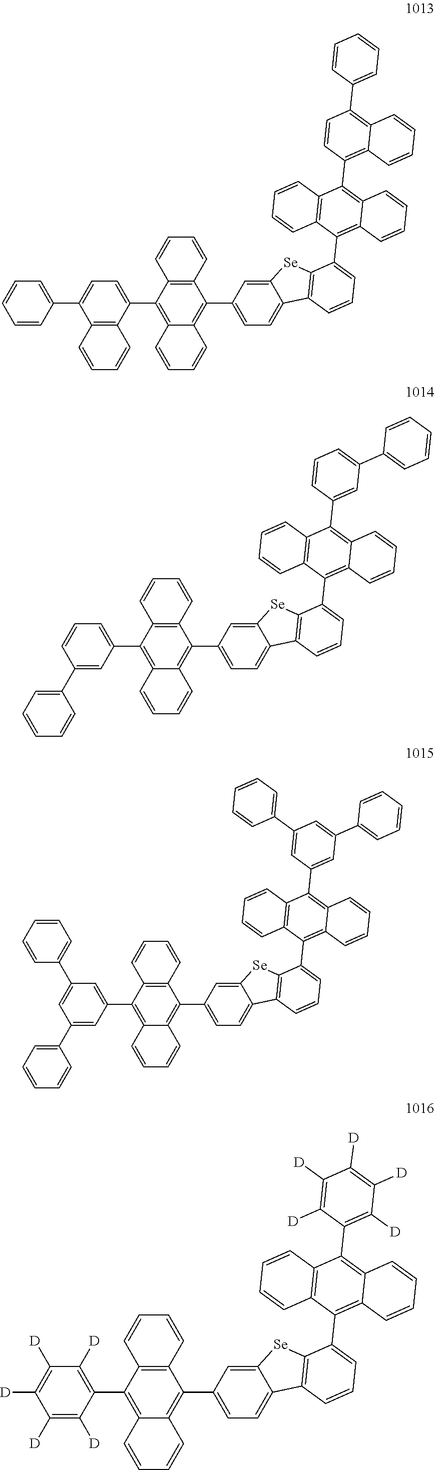

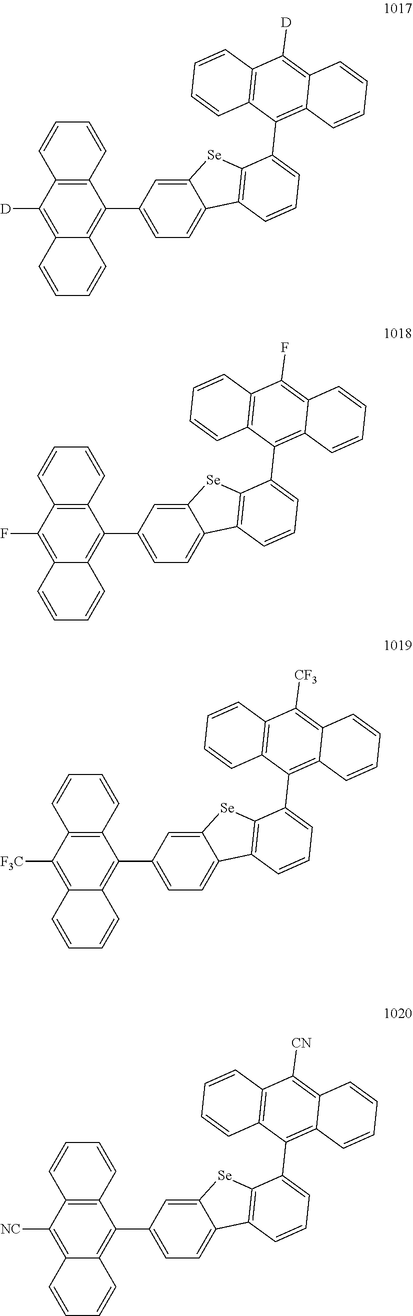 [SMILES: [2H]c1c2ccccc2c(-c2ccc3c(c2)[se]c2c(-c4c5ccccc5c([2H])c5ccccc45)cccc23)c2ccccc12 ; Fc1c2ccccc2c(-c2ccc3c(c2)[se]c2c(-c4c5ccccc5c(F)c5ccccc45)cccc23)c2ccccc12 ; FC(F)(F)c1c2ccccc2c(-c2ccc3c(c2)[se]c2c(-c4c5ccccc5c(C(F)(F)F)c5ccccc45)cccc23)c2ccccc12 ; N#Cc1c2ccccc2c(-c2ccc3c(c2)[se]c2c(-c4c5ccccc5c(C#N)c5ccccc45)cccc23)c2ccccc12]

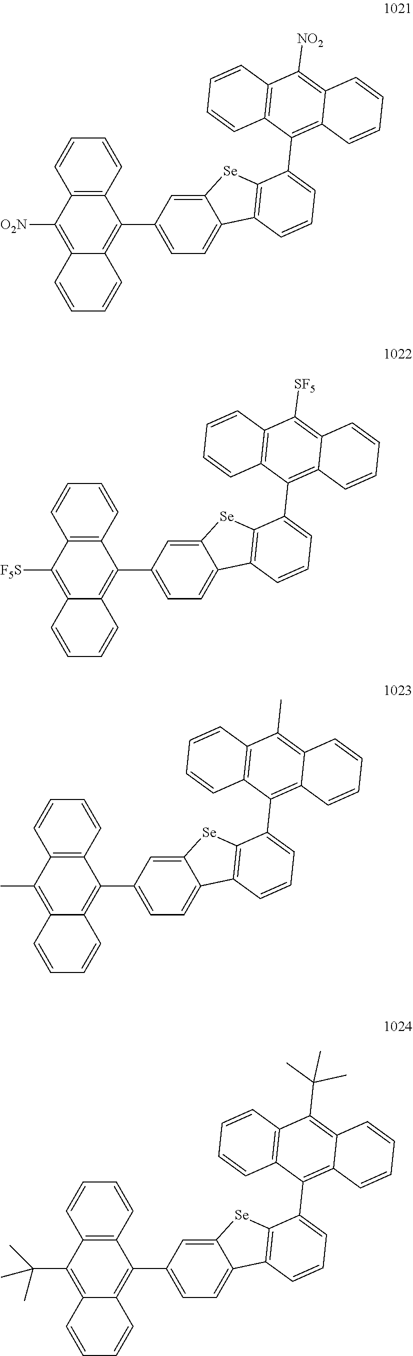

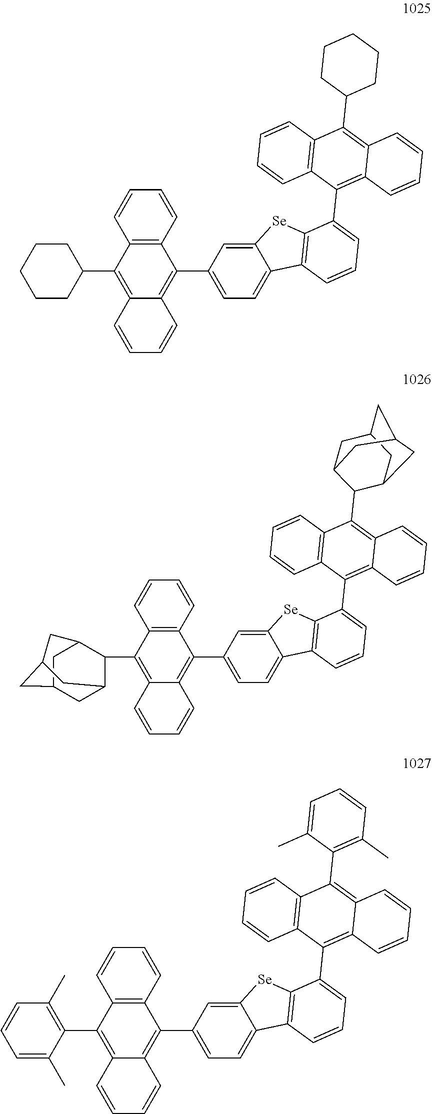 [SMILES: c1ccc2c(C3CCCCC3)c3ccccc3c(-c3ccc4c(c3)[se]c3c(-c5c6ccccc6c(C6CCCCC6)c6ccccc56)cccc34)c2c1 ; c1ccc2c(C3C4CC5CC(C4)CC3C5)c3ccccc3c(-c3ccc4c(c3)[se]c3c(-c5c6ccccc6c(C6C7CC8CC(C7)CC6C8)c6ccccc56)cccc34)c2c1 ; Cc1cccc(C)c1-c1c2ccccc2c(-c2ccc3c(c2)[se]c2c(-c4c5ccccc5c(-c5c(C)cccc5C)c5ccccc45)cccc23)c2ccccc12]

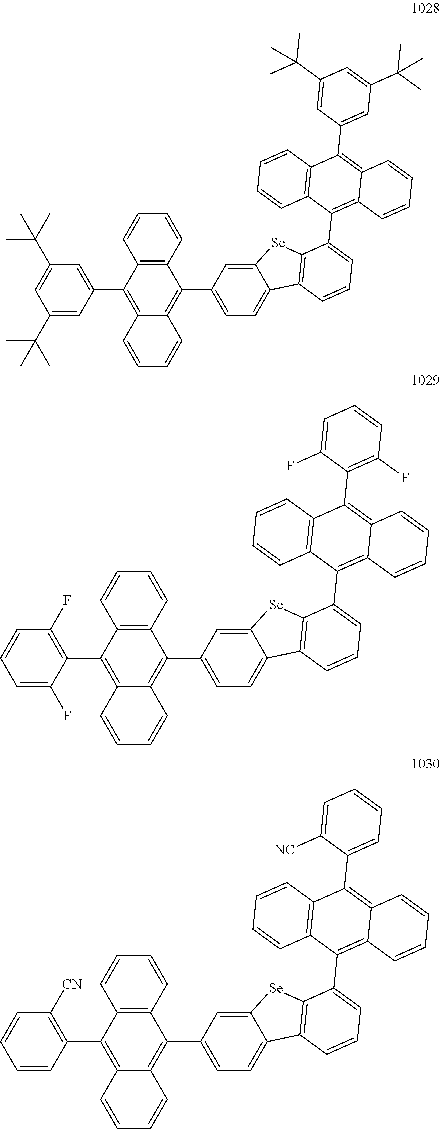 [SMILES: CC(C)(C)c1cc(-c2c3ccccc3c(-c3ccc4c(c3)[se]c3c(-c5c6ccccc6c(-c6cc(C(C)(C)C)cc(C(C)(C)C)c6)c6ccccc56)cccc34)c3ccccc23)cc(C(C)(C)C)c1 ; Fc1cccc(F)c1-c1c2ccccc2c(-c2ccc3c(c2)[se]c2c(-c4c5ccccc5c(-c5c(F)cccc5F)c5ccccc45)cccc23)c2ccccc12 ; N#Cc1ccccc1-c1c2ccccc2c(-c2ccc3c(c2)[se]c2c(-c4c5ccccc5c(-c5ccccc5C#N)c5ccccc45)cccc23)c2ccccc12]

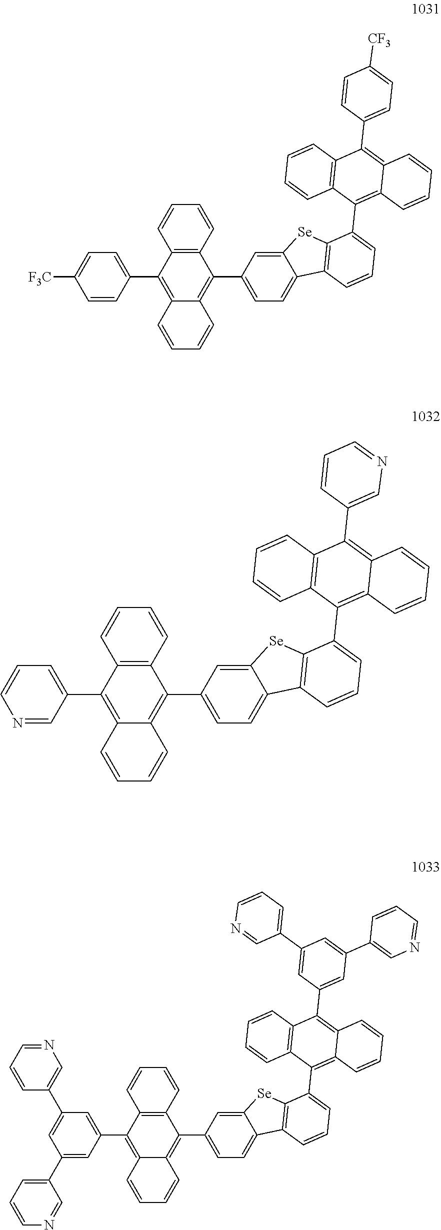 [SMILES: FC(F)(F)c1ccc(-c2c3ccccc3c(-c3ccc4c(c3)[se]c3c(-c5c6ccccc6c(-c6ccc(C(F)(F)F)cc6)c6ccccc56)cccc34)c3ccccc23)cc1 ; c1cncc(-c2c3ccccc3c(-c3ccc4c(c3)[se]c3c(-c5c6ccccc6c(-c6cccnc6)c6ccccc56)cccc34)c3ccccc23)c1 ; c1cncc(-c2cc(-c3cccnc3)cc(-c3c4ccccc4c(-c4ccc5c(c4)[se]c4c(-c6c7ccccc7c(-c7cc(-c8cccnc8)cc(-c8cccnc8)c7)c7ccccc67)cccc45)c4ccccc34)c2)c1]

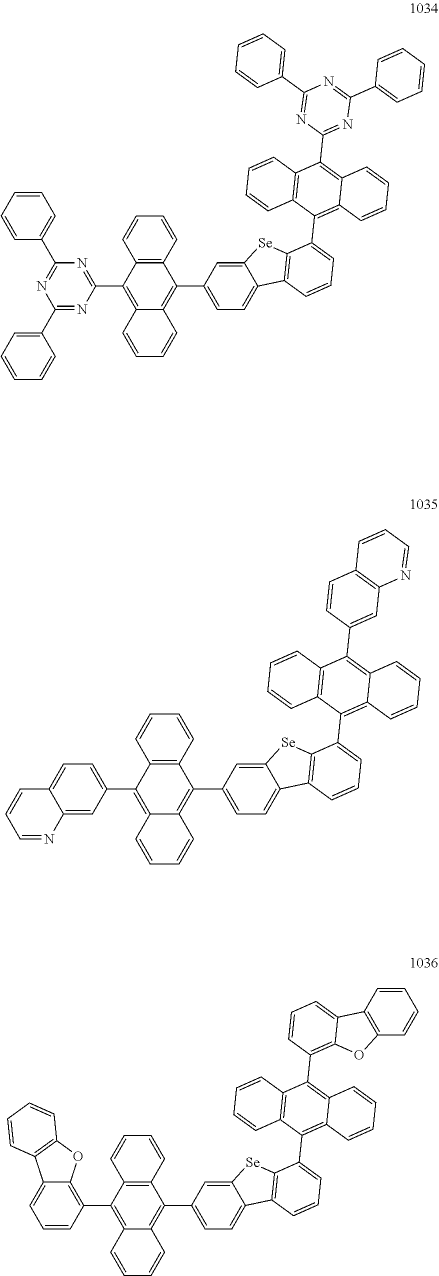 [SMILES: c1ccc(-c2nc(-c3ccccc3)nc(-c3c4ccccc4c(-c4ccc5c(c4)[se]c4c(-c6c7ccccc7c(-c7nc(-c8ccccc8)nc(-c8ccccc8)n7)c7ccccc67)cccc45)c4ccccc34)n2)cc1 ; c1cnc2cc(-c3c4ccccc4c(-c4ccc5c(c4)[se]c4c(-c6c7ccccc7c(-c7ccc8cccnc8c7)c7ccccc67)cccc45)c4ccccc34)ccc2c1 ; c1ccc2c(c1)oc1c(-c3c4ccccc4c(-c4ccc5c(c4)[se]c4c(-c6c7ccccc7c(-c7cccc8c7oc7ccccc78)c7ccccc67)cccc45)c4ccccc34)cccc12]

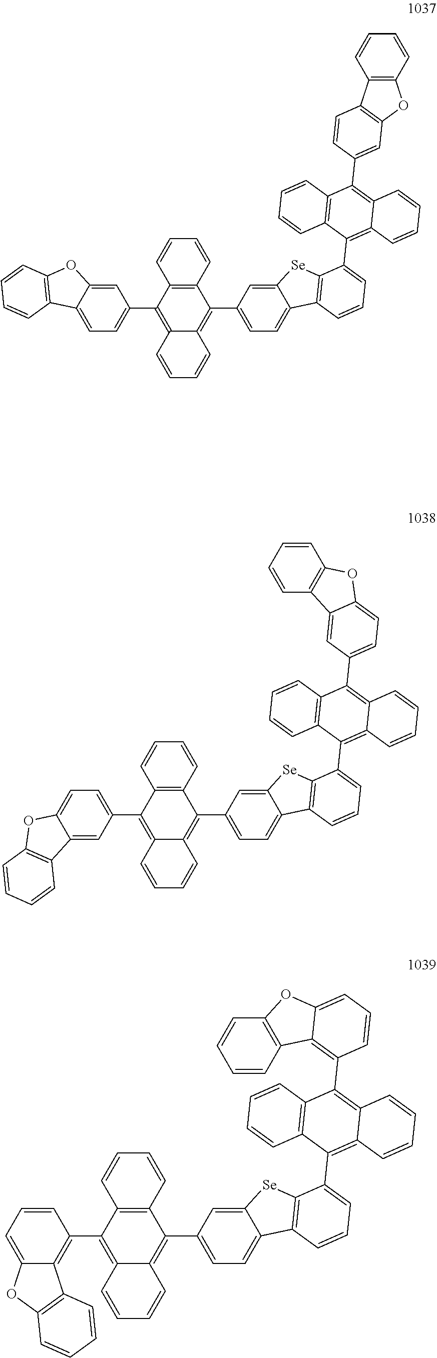 [SMILES: c1ccc2c(c1)oc1cc(-c3c4ccccc4c(-c4ccc5c(c4)[se]c4c(-c6c7ccccc7c(-c7ccc8c(c7)oc7ccccc78)c7ccccc67)cccc45)c4ccccc34)ccc12 ; c1ccc2c(c1)oc1ccc(-c3c4ccccc4c(-c4ccc5c(c4)[se]c4c(-c6c7ccccc7c(-c7ccc8oc9ccccc9c8c7)c7ccccc67)cccc45)c4ccccc34)cc12 ; c1ccc2c(c1)oc1cccc(-c3c4ccccc4c(-c4ccc5c(c4)[se]c4c(-c6c7ccccc7c(-c7cccc8oc9ccccc9c78)c7ccccc67)cccc45)c4ccccc34)c12]

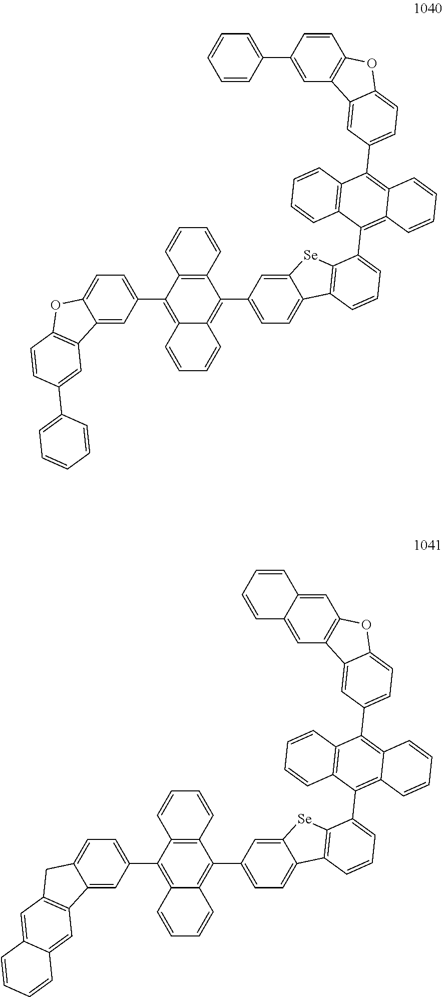 [SMILES: c1ccc(-c2ccc3oc4ccc(-c5c6ccccc6c(-c6ccc7c(c6)[se]c6c(-c8c9ccccc9c(-c9ccc%10oc%11ccc(-c%12ccccc%12)cc%11c%10c9)c9ccccc89)cccc67)c6ccccc56)cc4c3c2)cc1 ; c1ccc2cc3c(cc2c1)Cc1ccc(-c2c4ccccc4c(-c4ccc5c(c4)[se]c4c(-c6c7ccccc7c(-c7ccc8oc9cc%10ccccc%10cc9c8c7)c7ccccc67)cccc45)c4ccccc24)cc1-3]

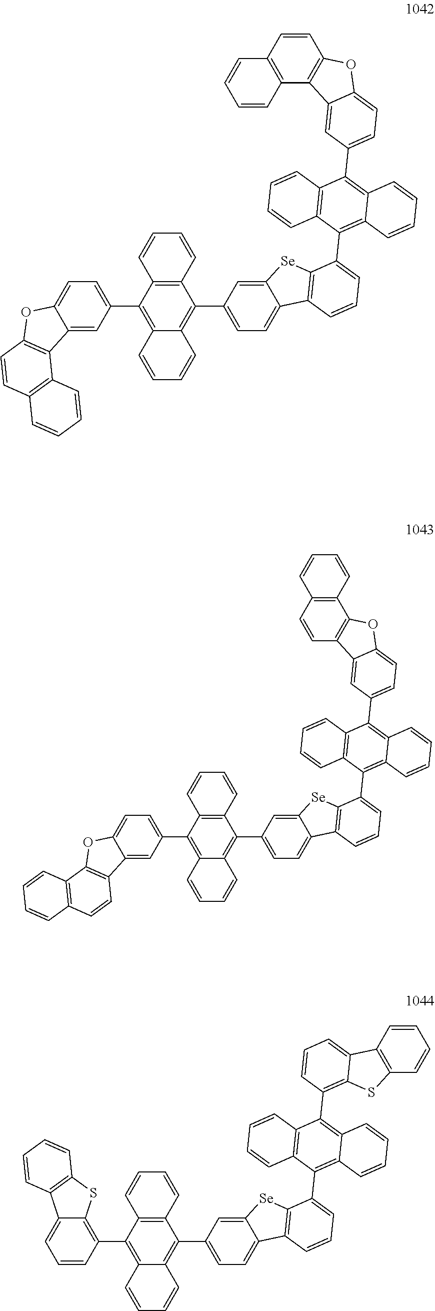 [SMILES: c1ccc2c(c1)ccc1oc3ccc(-c4c5ccccc5c(-c5ccc6c(c5)[se]c5c(-c7c8ccccc8c(-c8ccc9oc%10ccc%11ccccc%11c%10c9c8)c8ccccc78)cccc56)c5ccccc45)cc3c12 ; c1ccc2c(c1)ccc1c3cc(-c4c5ccccc5c(-c5ccc6c(c5)[se]c5c(-c7c8ccccc8c(-c8ccc9oc%10c%11ccccc%11ccc%10c9c8)c8ccccc78)cccc56)c5ccccc45)ccc3oc21 ; c1ccc2c(c1)sc1c(-c3c4ccccc4c(-c4ccc5c(c4)[se]c4c(-c6c7ccccc7c(-c7cccc8c7sc7ccccc78)c7ccccc67)cccc45)c4ccccc34)cccc12]

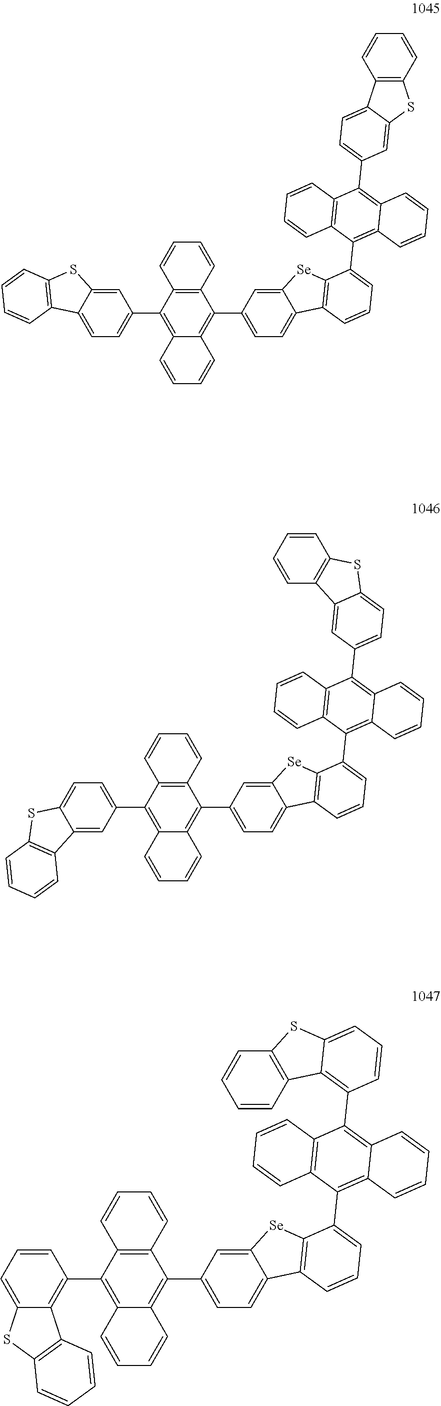 [SMILES: c1ccc2c(c1)sc1cc(-c3c4ccccc4c(-c4ccc5c(c4)[se]c4c(-c6c7ccccc7c(-c7ccc8c(c7)sc7ccccc78)c7ccccc67)cccc45)c4ccccc34)ccc12 ; c1ccc2c(c1)sc1ccc(-c3c4ccccc4c(-c4ccc5c(c4)[se]c4c(-c6c7ccccc7c(-c7ccc8sc9ccccc9c8c7)c7ccccc67)cccc45)c4ccccc34)cc12 ; c1ccc2c(c1)sc1cccc(-c3c4ccccc4c(-c4ccc5c(c4)[se]c4c(-c6c7ccccc7c(-c7cccc8sc9ccccc9c78)c7ccccc67)cccc45)c4ccccc34)c12]

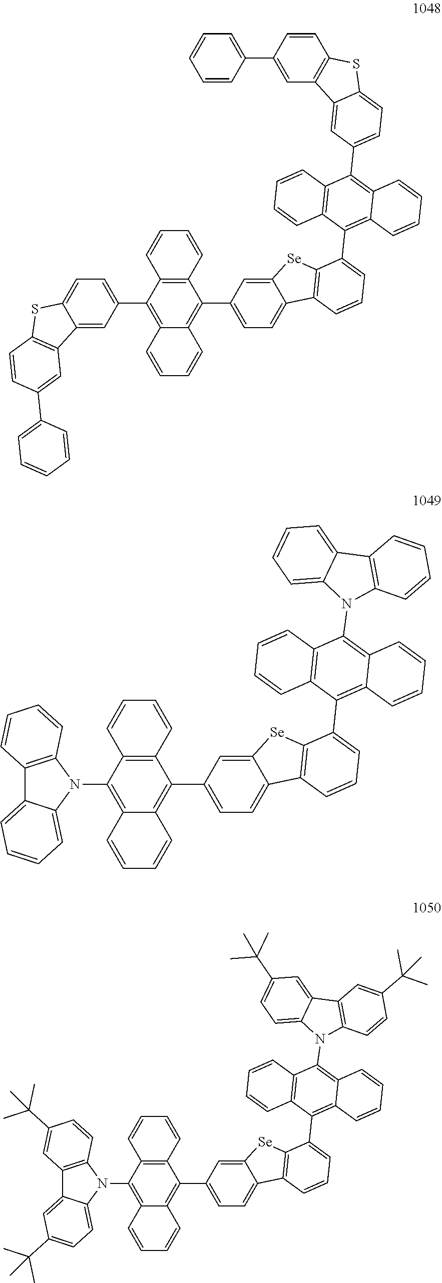 [SMILES: c1ccc(-c2ccc3sc4ccc(-c5c6ccccc6c(-c6ccc7c(c6)[se]c6c(-c8c9ccccc9c(-c9ccc%10sc%11ccc(-c%12ccccc%12)cc%11c%10c9)c9ccccc89)cccc67)c6ccccc56)cc4c3c2)cc1 ; c1ccc2c(-n3c4ccccc4c4ccccc43)c3ccccc3c(-c3ccc4c(c3)[se]c3c(-c5c6ccccc6c(-n6c7ccccc7c7ccccc76)c6ccccc56)cccc34)c2c1 ; CC(C)(C)c1ccc2c(c1)c1cc(C(C)(C)C)ccc1n2-c1c2ccccc2c(-c2ccc3c(c2)[se]c2c(-c4c5ccccc5c(-n5c6ccc(C(C)(C)C)cc6c6cc(C(C)(C)C)ccc65)c5ccccc45)cccc23)c2ccccc12]

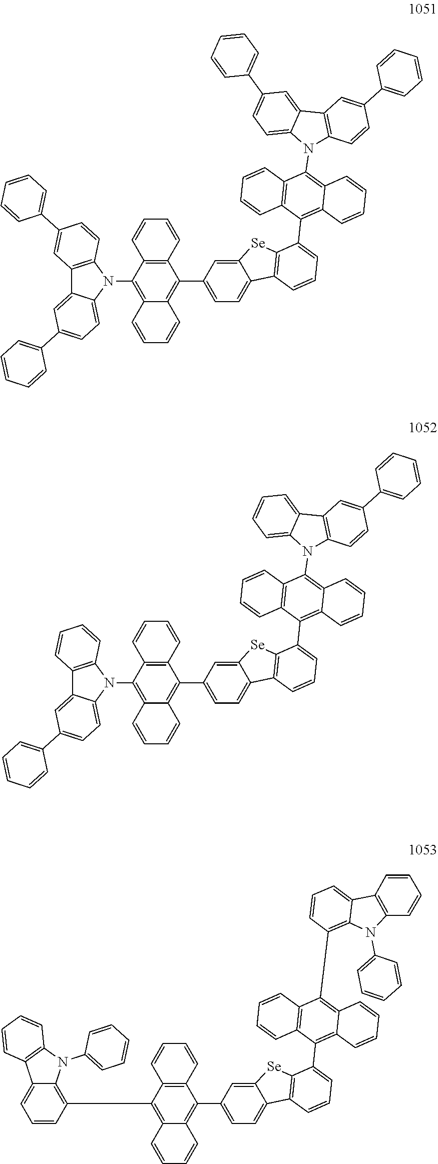 [SMILES: c1ccc(-c2ccc3c(c2)c2cc(-c4ccccc4)ccc2n3-c2c3ccccc3c(-c3ccc4c(c3)[se]c3c(-c5c6ccccc6c(-n6c7ccc(-c8ccccc8)cc7c7cc(-c8ccccc8)ccc76)c6ccccc56)cccc34)c3ccccc23)cc1 ; c1ccc(-c2ccc3c(c2)c2ccccc2n3-c2c3ccccc3c(-c3ccc4c(c3)[se]c3c(-c5c6ccccc6c(-n6c7ccccc7c7cc(-c8ccccc8)ccc76)c6ccccc56)cccc34)c3ccccc23)cc1 ; c1ccc(-n2c3ccccc3c3cccc(-c4c5ccccc5c(-c5ccc6c(c5)[se]c5c(-c7c8ccccc8c(-c8cccc9c%10ccccc%10n(-c%10ccccc%10)c89)c8ccccc78)cccc56)c5ccccc45)c32)cc1]

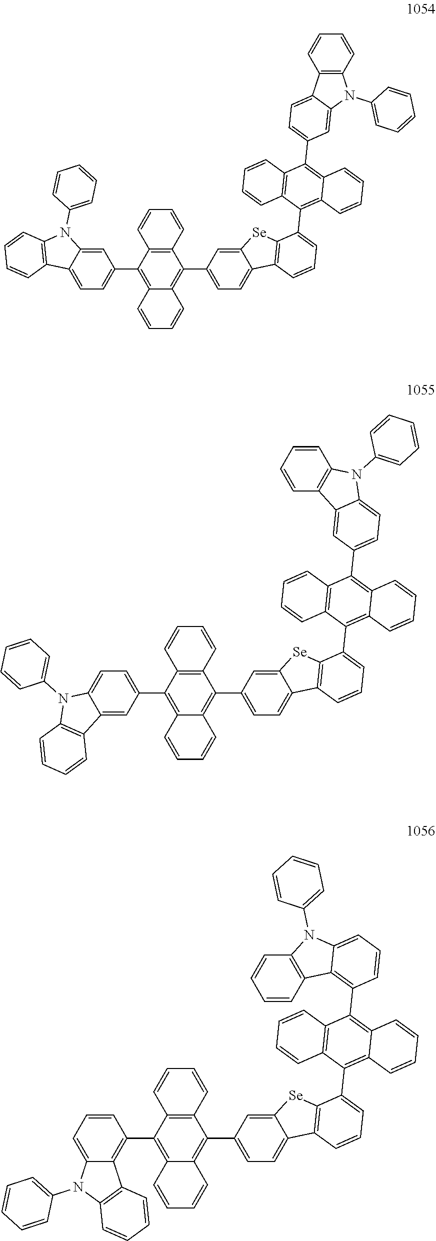 [SMILES: c1ccc(-n2c3ccccc3c3ccc(-c4c5ccccc5c(-c5ccc6c(c5)[se]c5c(-c7c8ccccc8c(-c8ccc9c%10ccccc%10n(-c%10ccccc%10)c9c8)c8ccccc78)cccc56)c5ccccc45)cc32)cc1 ; c1ccc(-n2c3ccccc3c3cc(-c4c5ccccc5c(-c5ccc6c(c5)[se]c5c(-c7c8ccccc8c(-c8ccc9c(c8)c8ccccc8n9-c8ccccc8)c8ccccc78)cccc56)c5ccccc45)ccc32)cc1 ; c1ccc(-n2c3ccccc3c3c(-c4c5ccccc5c(-c5ccc6c(c5)[se]c5c(-c7c8ccccc8c(-c8cccc9c8c8ccccc8n9-c8ccccc8)c8ccccc78)cccc56)c5ccccc45)cccc32)cc1]

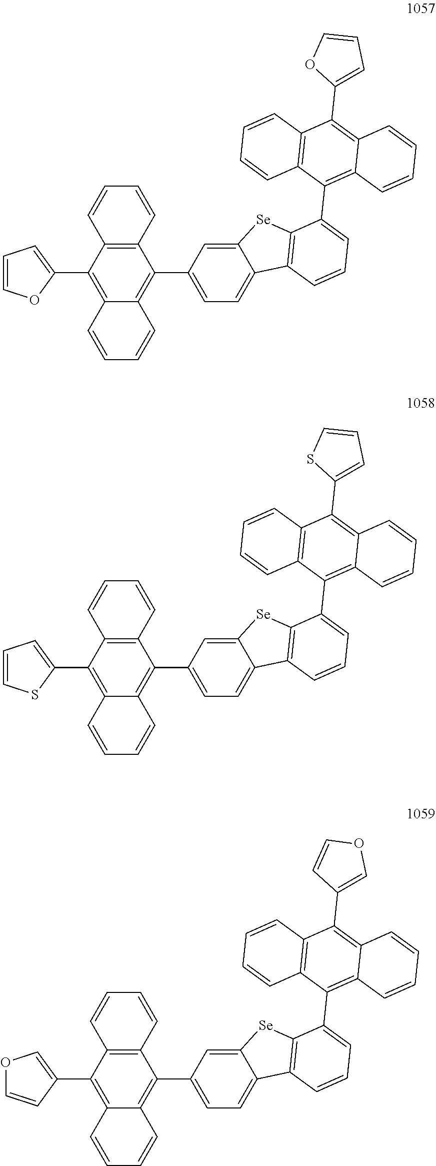 [SMILES: c1coc(-c2c3ccccc3c(-c3ccc4c(c3)[se]c3c(-c5c6ccccc6c(-c6ccco6)c6ccccc56)cccc34)c3ccccc23)c1 ; c1csc(-c2c3ccccc3c(-c3ccc4c(c3)[se]c3c(-c5c6ccccc6c(-c6cccs6)c6ccccc56)cccc34)c3ccccc23)c1 ; c1ccc2c(-c3ccc4c(c3)[se]c3c(-c5c6ccccc6c(-c6ccoc6)c6ccccc56)cccc34)c3ccccc3c(-c3ccoc3)c2c1]

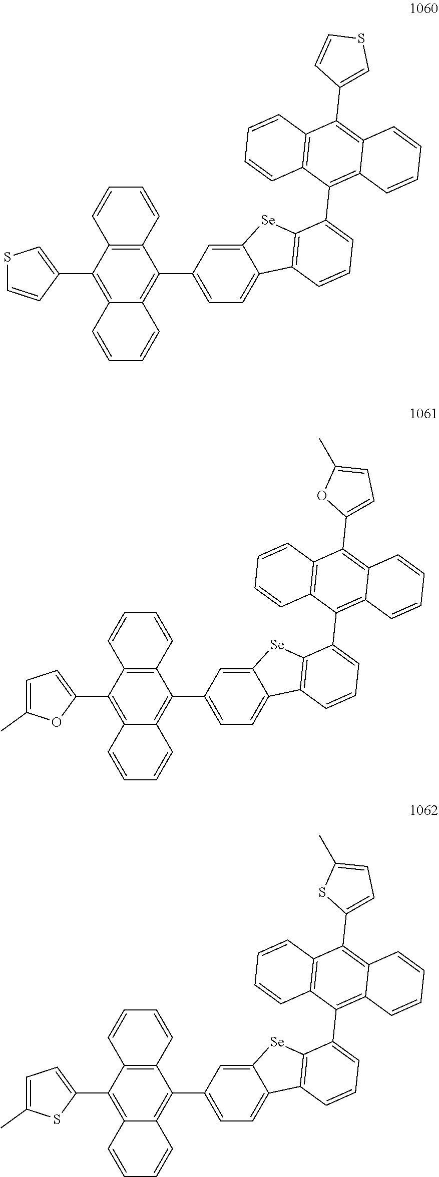 [SMILES: c1ccc2c(-c3ccc4c(c3)[se]c3c(-c5c6ccccc6c(-c6ccsc6)c6ccccc56)cccc34)c3ccccc3c(-c3ccsc3)c2c1 ; Cc1ccc(-c2c3ccccc3c(-c3ccc4c(c3)[se]c3c(-c5c6ccccc6c(-c6ccc(C)o6)c6ccccc56)cccc34)c3ccccc23)o1 ; Cc1ccc(-c2c3ccccc3c(-c3ccc4c(c3)[se]c3c(-c5c6ccccc6c(-c6ccc(C)s6)c6ccccc56)cccc34)c3ccccc23)s1]

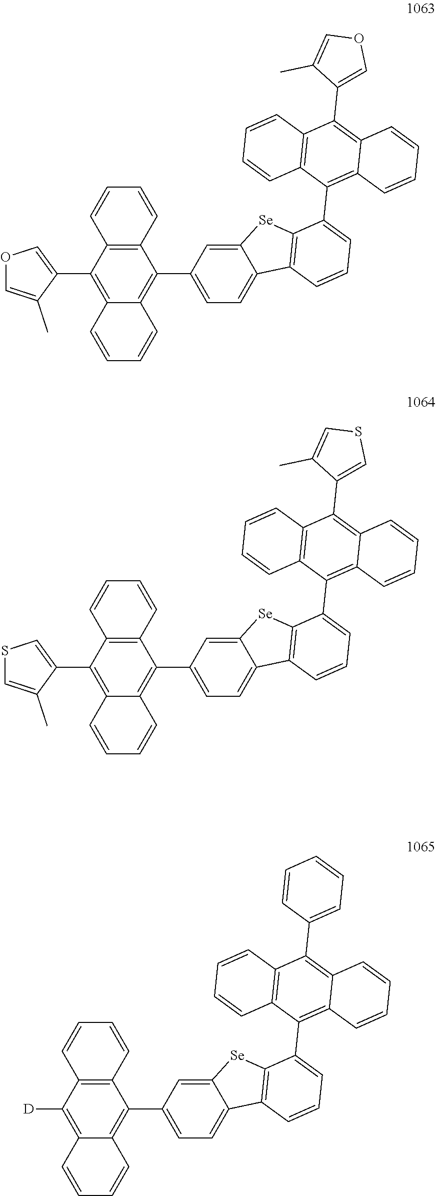 [SMILES: Cc1cocc1-c1c2ccccc2c(-c2ccc3c(c2)[se]c2c(-c4c5ccccc5c(-c5cocc5C)c5ccccc45)cccc23)c2ccccc12 ; Cc1cscc1-c1c2ccccc2c(-c2ccc3c(c2)[se]c2c(-c4c5ccccc5c(-c5cscc5C)c5ccccc45)cccc23)c2ccccc12 ; [2H]c1c2ccccc2c(-c2ccc3c(c2)[se]c2c(-c4c5ccccc5c(-c5ccccc5)c5ccccc45)cccc23)c2ccccc12]

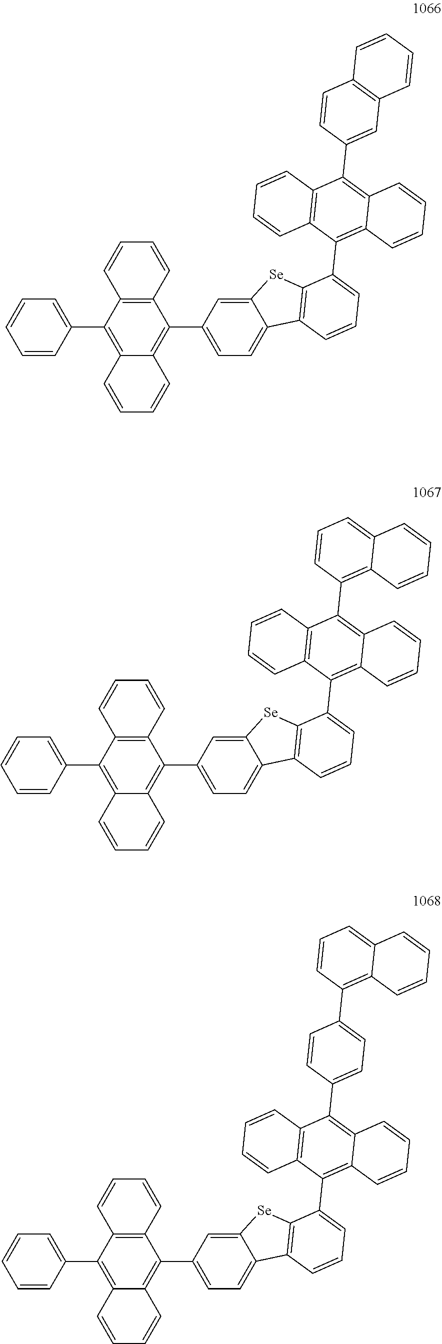 [SMILES: c1ccc(-c2c3ccccc3c(-c3ccc4c(c3)[se]c3c(-c5c6ccccc6c(-c6ccc7ccccc7c6)c6ccccc56)cccc34)c3ccccc23)cc1 ; c1ccc(-c2c3ccccc3c(-c3ccc4c(c3)[se]c3c(-c5c6ccccc6c(-c6cccc7ccccc67)c6ccccc56)cccc34)c3ccccc23)cc1 ; c1ccc(-c2c3ccccc3c(-c3ccc4c(c3)[se]c3c(-c5c6ccccc6c(-c6ccc(-c7cccc8ccccc78)cc6)c6ccccc56)cccc34)c3ccccc23)cc1]

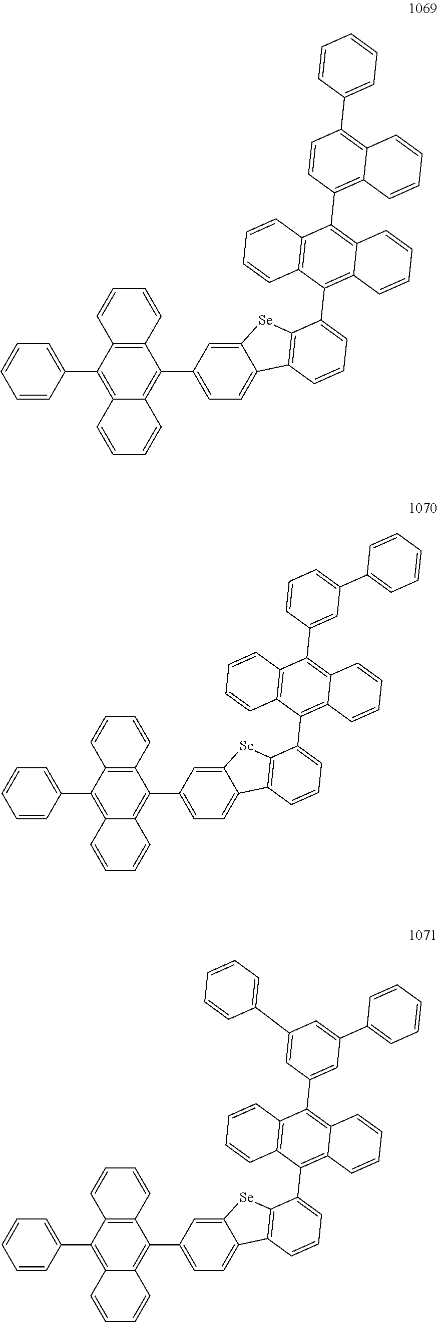 [SMILES: c1ccc(-c2ccc(-c3c4ccccc4c(-c4cccc5c4[se]c4cc(-c6c7ccccc7c(-c7ccccc7)c7ccccc67)ccc45)c4ccccc34)c3ccccc23)cc1 ; c1ccc(-c2cccc(-c3c4ccccc4c(-c4cccc5c4[se]c4cc(-c6c7ccccc7c(-c7ccccc7)c7ccccc67)ccc45)c4ccccc34)c2)cc1 ; c1ccc(-c2cc(-c3ccccc3)cc(-c3c4ccccc4c(-c4cccc5c4[se]c4cc(-c6c7ccccc7c(-c7ccccc7)c7ccccc67)ccc45)c4ccccc34)c2)cc1]

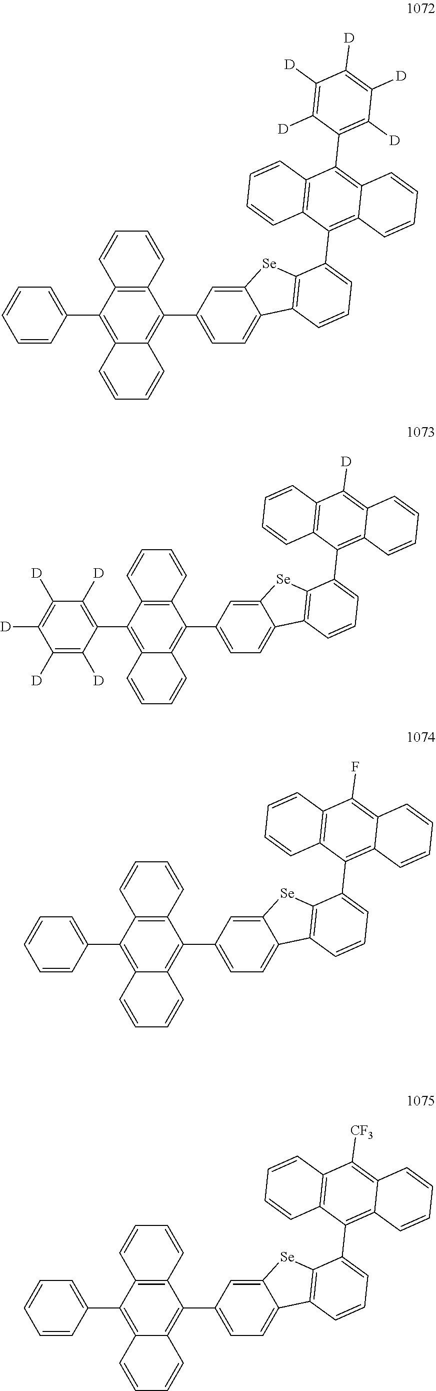 [SMILES: [2H]c1c([2H])c([2H])c(-c2c3ccccc3c(-c3cccc4c3[se]c3cc(-c5c6ccccc6c(-c6ccccc6)c6ccccc56)ccc34)c3ccccc23)c([2H])c1[2H] ; [2H]c1c([2H])c([2H])c(-c2c3ccccc3c(-c3ccc4c(c3)[se]c3c(-c5c6ccccc6c([2H])c6ccccc56)cccc34)c3ccccc23)c([2H])c1[2H] ; Fc1c2ccccc2c(-c2cccc3c2[se]c2cc(-c4c5ccccc5c(-c5ccccc5)c5ccccc45)ccc23)c2ccccc12 ; FC(F)(F)c1c2ccccc2c(-c2cccc3c2[se]c2cc(-c4c5ccccc5c(-c5ccccc5)c5ccccc45)ccc23)c2ccccc12]

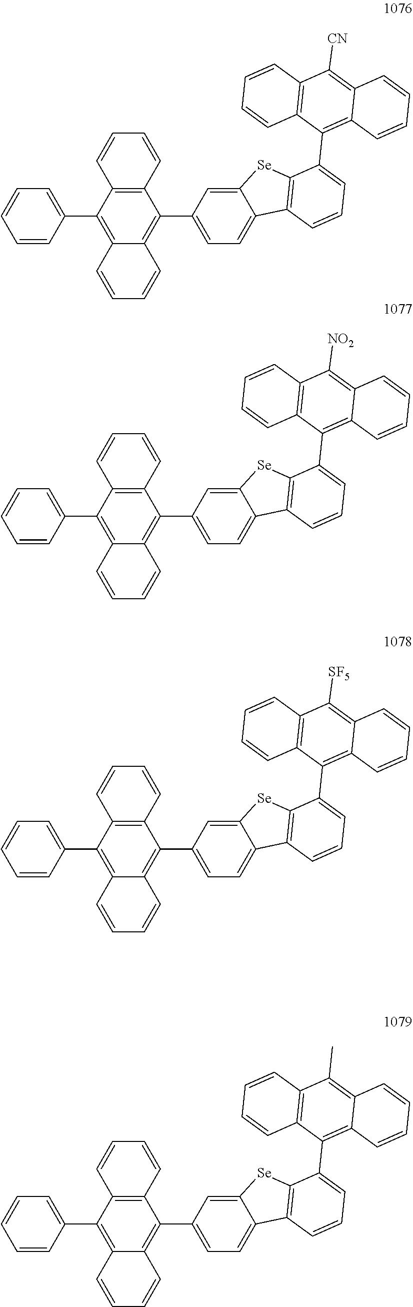

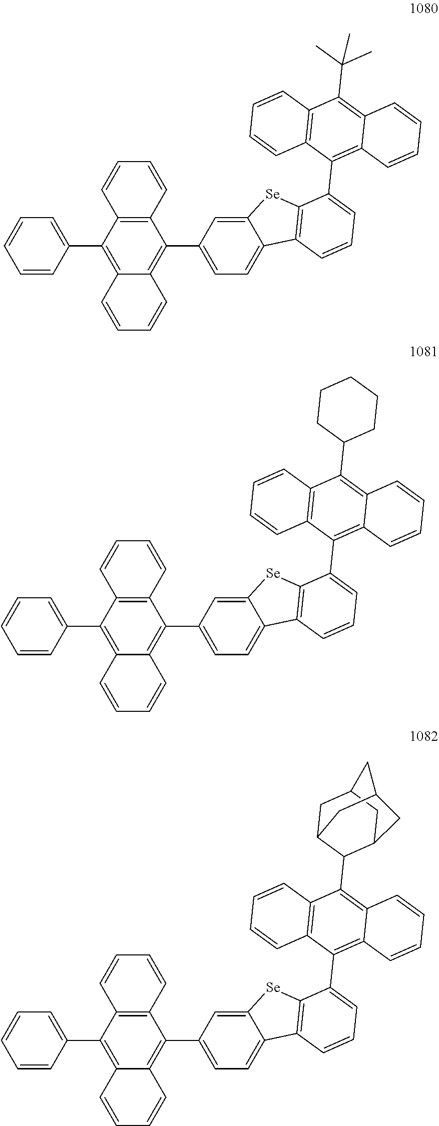 [SMILES: CC(C)(C)c1c2ccccc2c(-c2cccc3c2[se]c2cc(-c4c5ccccc5c(-c5ccccc5)c5ccccc45)ccc23)c2ccccc12 ; c1ccc(-c2c3ccccc3c(-c3ccc4c(c3)[se]c3c(-c5c6ccccc6c(C6CCCCC6)c6ccccc56)cccc34)c3ccccc23)cc1 ; c1ccc(-c2c3ccccc3c(-c3ccc4c(c3)[se]c3c(-c5c6ccccc6c(C6C7CC8CC(C7)CC6C8)c6ccccc56)cccc34)c3ccccc23)cc1]

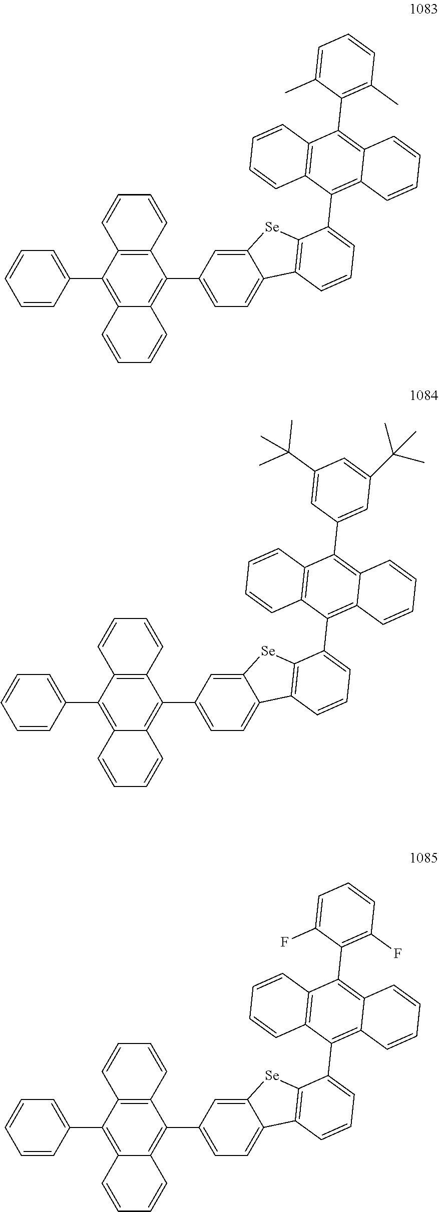 [SMILES: Cc1cccc(C)c1-c1c2ccccc2c(-c2cccc3c2[se]c2cc(-c4c5ccccc5c(-c5ccccc5)c5ccccc45)ccc23)c2ccccc12 ; CC(C)(C)c1cc(-c2c3ccccc3c(-c3cccc4c3[se]c3cc(-c5c6ccccc6c(-c6ccccc6)c6ccccc56)ccc34)c3ccccc23)cc(C(C)(C)C)c1 ; Fc1cccc(F)c1-c1c2ccccc2c(-c2cccc3c2[se]c2cc(-c4c5ccccc5c(-c5ccccc5)c5ccccc45)ccc23)c2ccccc12]

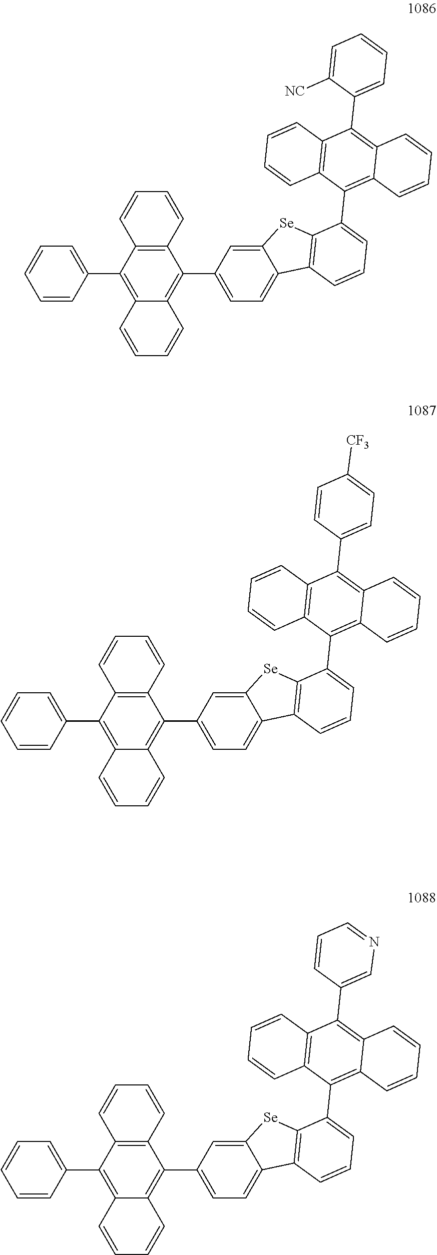 [SMILES: N#Cc1ccccc1-c1c2ccccc2c(-c2cccc3c2[se]c2cc(-c4c5ccccc5c(-c5ccccc5)c5ccccc45)ccc23)c2ccccc12 ; FC(F)(F)c1ccc(-c2c3ccccc3c(-c3cccc4c3[se]c3cc(-c5c6ccccc6c(-c6ccccc6)c6ccccc56)ccc34)c3ccccc23)cc1 ; c1ccc(-c2c3ccccc3c(-c3ccc4c(c3)[se]c3c(-c5c6ccccc6c(-c6cccnc6)c6ccccc56)cccc34)c3ccccc23)cc1]

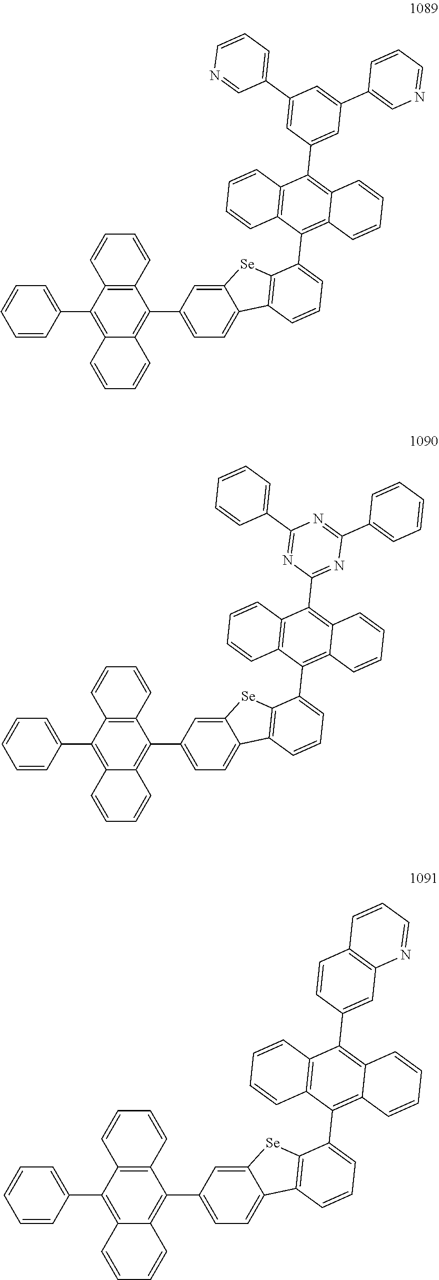 [SMILES: c1ccc(-c2c3ccccc3c(-c3ccc4c(c3)[se]c3c(-c5c6ccccc6c(-c6cc(-c7cccnc7)cc(-c7cccnc7)c6)c6ccccc56)cccc34)c3ccccc23)cc1 ; c1ccc(-c2nc(-c3ccccc3)nc(-c3c4ccccc4c(-c4cccc5c4[se]c4cc(-c6c7ccccc7c(-c7ccccc7)c7ccccc67)ccc45)c4ccccc34)n2)cc1 ; c1ccc(-c2c3ccccc3c(-c3ccc4c(c3)[se]c3c(-c5c6ccccc6c(-c6ccc7cccnc7c6)c6ccccc56)cccc34)c3ccccc23)cc1]

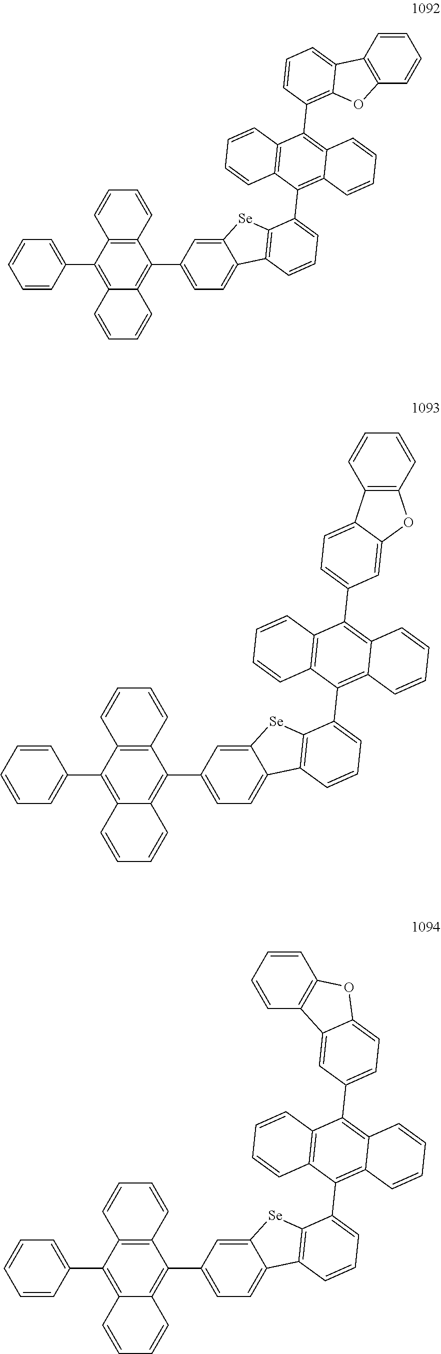 [SMILES: c1ccc(-c2c3ccccc3c(-c3ccc4c(c3)[se]c3c(-c5c6ccccc6c(-c6cccc7c6oc6ccccc67)c6ccccc56)cccc34)c3ccccc23)cc1 ; c1ccc(-c2c3ccccc3c(-c3ccc4c(c3)[se]c3c(-c5c6ccccc6c(-c6ccc7c(c6)oc6ccccc67)c6ccccc56)cccc34)c3ccccc23)cc1 ; c1ccc(-c2c3ccccc3c(-c3ccc4c(c3)[se]c3c(-c5c6ccccc6c(-c6ccc7oc8ccccc8c7c6)c6ccccc56)cccc34)c3ccccc23)cc1]

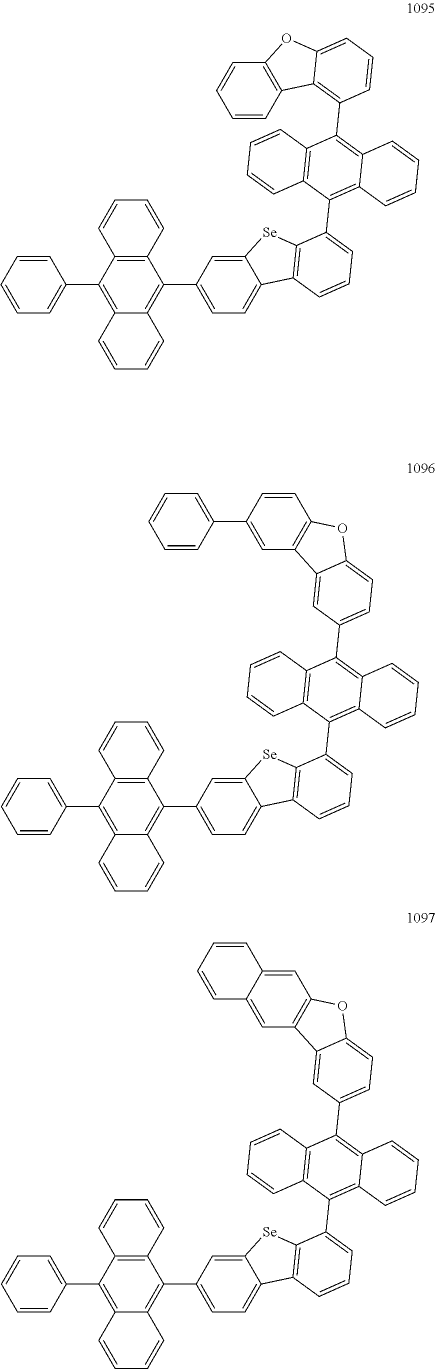 [SMILES: c1ccc(-c2c3ccccc3c(-c3ccc4c(c3)[se]c3c(-c5c6ccccc6c(-c6cccc7oc8ccccc8c67)c6ccccc56)cccc34)c3ccccc23)cc1 ; c1ccc(-c2ccc3oc4ccc(-c5c6ccccc6c(-c6cccc7c6[se]c6cc(-c8c9ccccc9c(-c9ccccc9)c9ccccc89)ccc67)c6ccccc56)cc4c3c2)cc1 ; c1ccc(-c2c3ccccc3c(-c3ccc4c(c3)[se]c3c(-c5c6ccccc6c(-c6ccc7oc8cc9ccccc9cc8c7c6)c6ccccc56)cccc34)c3ccccc23)cc1]

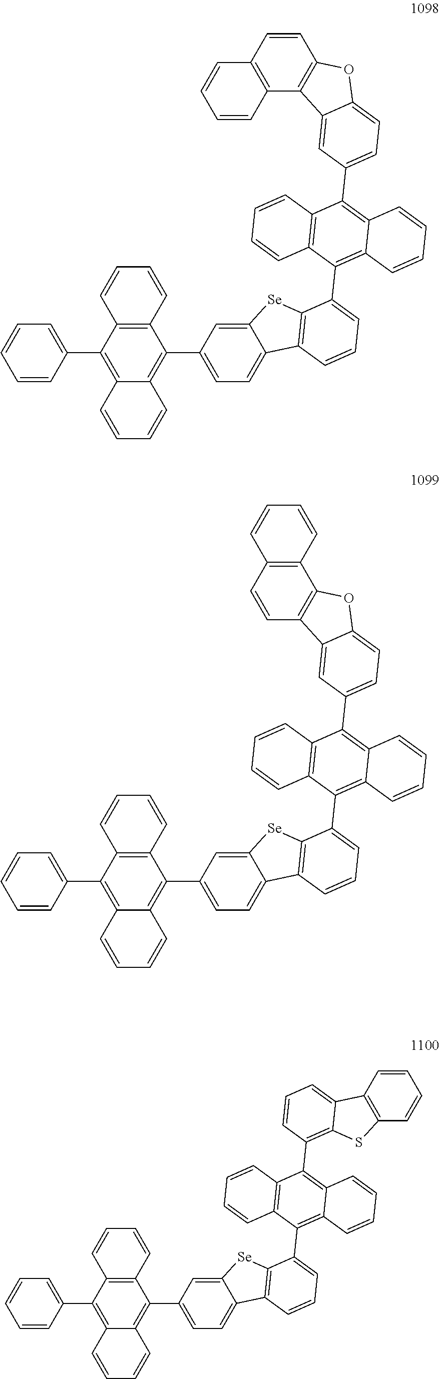 [SMILES: c1ccc(-c2c3ccccc3c(-c3ccc4c(c3)[se]c3c(-c5c6ccccc6c(-c6ccc7oc8ccc9ccccc9c8c7c6)c6ccccc56)cccc34)c3ccccc23)cc1 ; c1ccc(-c2c3ccccc3c(-c3ccc4c(c3)[se]c3c(-c5c6ccccc6c(-c6ccc7oc8c9ccccc9ccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7c6sc6ccccc67)c6ccccc56)cccc34)c3ccccc23)cc1]

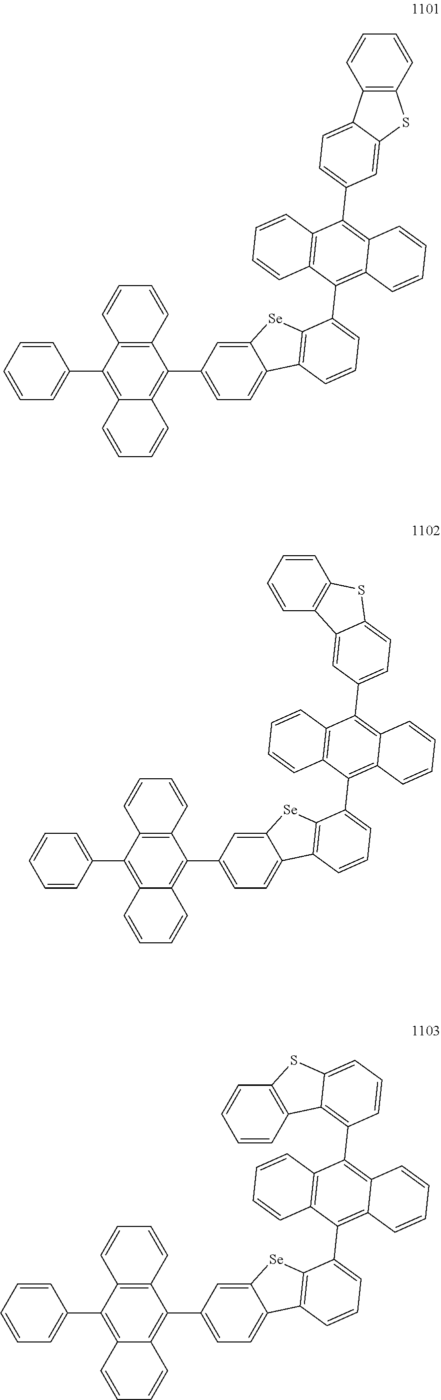 [SMILES: c1ccc(-c2c3ccccc3c(-c3ccc4c(c3)[se]c3c(-c5c6ccccc6c(-c6ccc7c(c6)sc6ccccc67)c6ccccc56)cccc34)c3ccccc23)cc1 ; c1ccc(-c2c3ccccc3c(-c3ccc4c(c3)[se]c3c(-c5c6ccccc6c(-c6ccc7sc8ccccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7sc8ccccc8c67)c6ccccc56)cccc34)c3ccccc23)cc1]

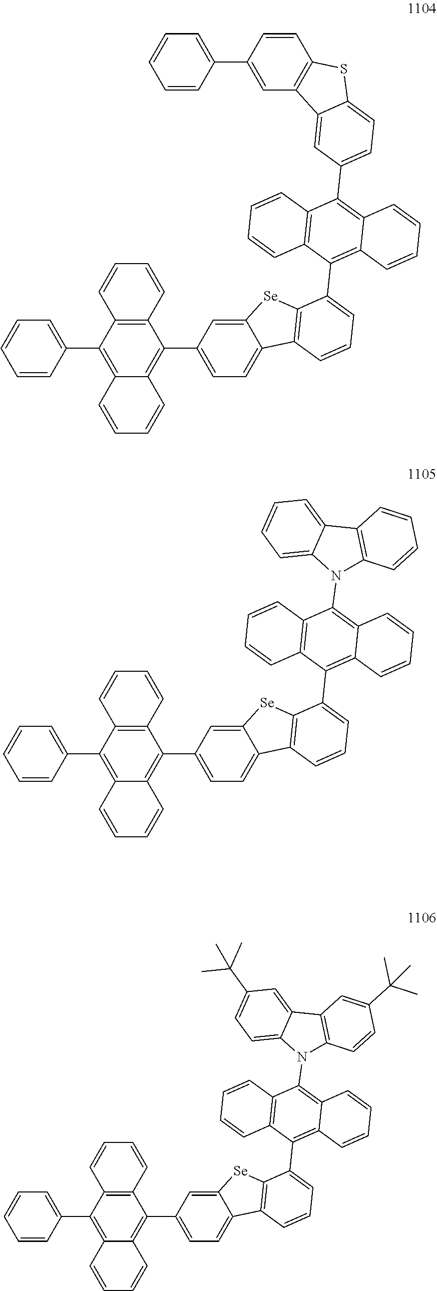 [SMILES: c1ccc(-c2ccc3sc4ccc(-c5c6ccccc6c(-c6cccc7c6[se]c6cc(-c8c9ccccc9c(-c9ccccc9)c9ccccc89)ccc67)c6ccccc56)cc4c3c2)cc1 ; c1ccc(-c2c3ccccc3c(-c3ccc4c(c3)[se]c3c(-c5c6ccccc6c(-n6c7ccccc7c7ccccc76)c6ccccc56)cccc34)c3ccccc23)cc1 ; CC(C)(C)c1ccc2c(c1)c1cc(C(C)(C)C)ccc1n2-c1c2ccccc2c(-c2cccc3c2[se]c2cc(-c4c5ccccc5c(-c5ccccc5)c5ccccc45)ccc23)c2ccccc12]

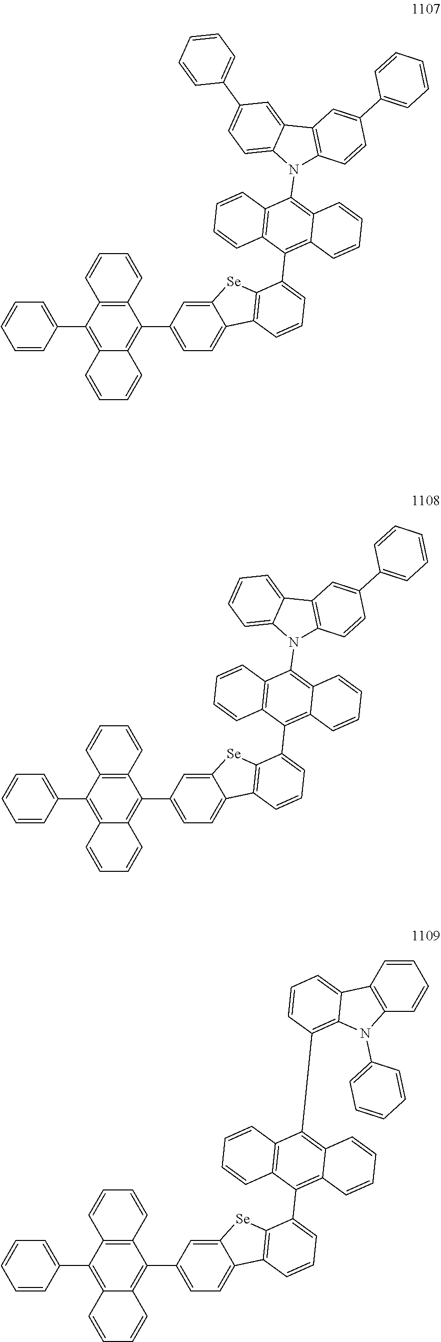 [SMILES: c1ccc(-c2ccc3c(c2)c2cc(-c4ccccc4)ccc2n3-c2c3ccccc3c(-c3cccc4c3[se]c3cc(-c5c6ccccc6c(-c6ccccc6)c6ccccc56)ccc34)c3ccccc23)cc1 ; c1ccc(-c2ccc3c(c2)c2ccccc2n3-c2c3ccccc3c(-c3cccc4c3[se]c3cc(-c5c6ccccc6c(-c6ccccc6)c6ccccc56)ccc34)c3ccccc23)cc1 ; c1ccc(-c2c3ccccc3c(-c3ccc4c(c3)[se]c3c(-c5c6ccccc6c(-c6cccc7c8ccccc8n(-c8ccccc8)c67)c6ccccc56)cccc34)c3ccccc23)cc1]

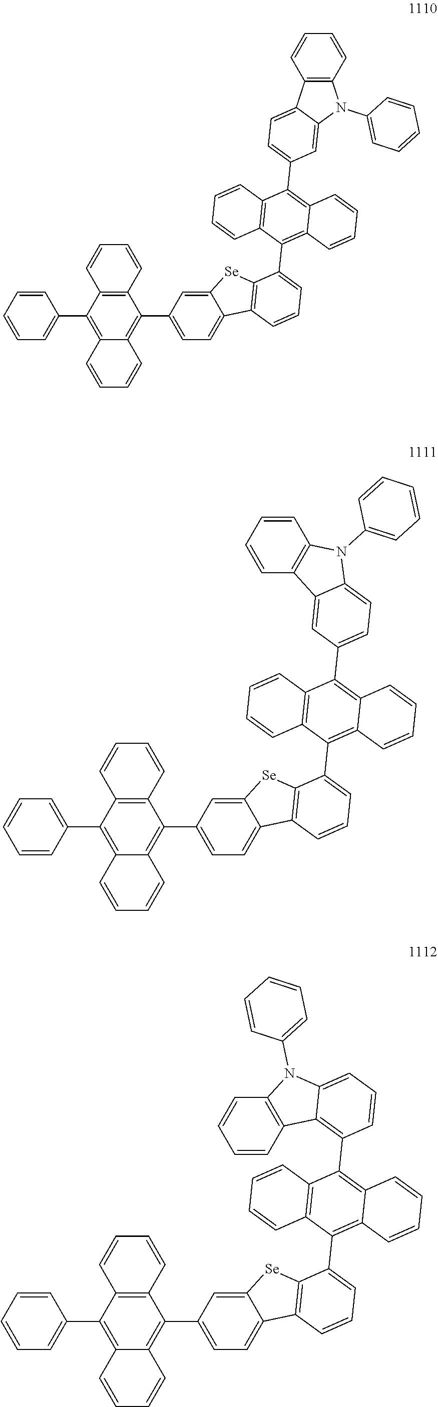 [SMILES: c1ccc(-c2c3ccccc3c(-c3ccc4c(c3)[se]c3c(-c5c6ccccc6c(-c6ccc7c8ccccc8n(-c8ccccc8)c7c6)c6ccccc56)cccc34)c3ccccc23)cc1 ; c1ccc(-c2c3ccccc3c(-c3ccc4c(c3)[se]c3c(-c5c6ccccc6c(-c6ccc7c(c6)c6ccccc6n7-c6ccccc6)c6ccccc56)cccc34)c3ccccc23)cc1 ; c1ccc(-c2c3ccccc3c(-c3ccc4c(c3)[se]c3c(-c5c6ccccc6c(-c6cccc7c6c6ccccc6n7-c6ccccc6)c6ccccc56)cccc34)c3ccccc23)cc1]

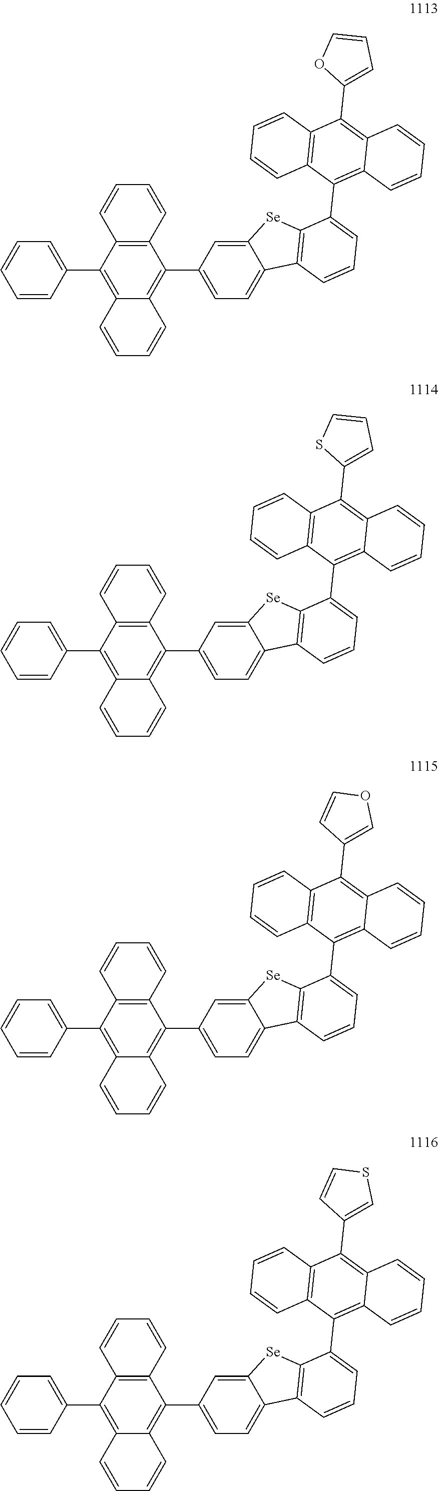 [SMILES: c1ccc(-c2c3ccccc3c(-c3ccc4c(c3)[se]c3c(-c5c6ccccc6c(-c6ccco6)c6ccccc56)cccc34)c3ccccc23)cc1 ; c1ccc(-c2c3ccccc3c(-c3ccc4c(c3)[se]c3c(-c5c6ccccc6c(-c6cccs6)c6ccccc56)cccc34)c3ccccc23)cc1 ; c1ccc(-c2c3ccccc3c(-c3ccc4c(c3)[se]c3c(-c5c6ccccc6c(-c6ccoc6)c6ccccc56)cccc34)c3ccccc23)cc1 ; c1ccc(-c2c3ccccc3c(-c3ccc4c(c3)[se]c3c(-c5c6ccccc6c(-c6ccsc6)c6ccccc56)cccc34)c3ccccc23)cc1]

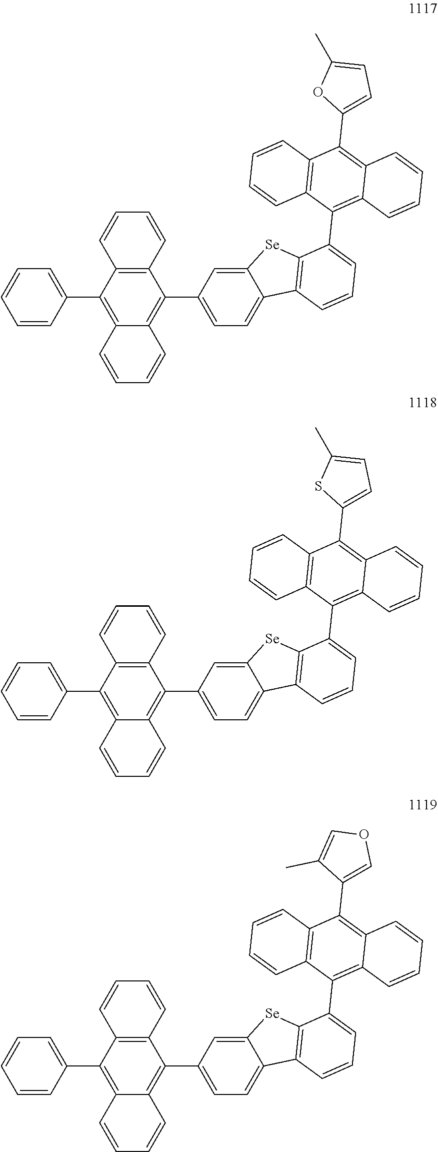 [SMILES: Cc1ccc(-c2c3ccccc3c(-c3cccc4c3[se]c3cc(-c5c6ccccc6c(-c6ccccc6)c6ccccc56)ccc34)c3ccccc23)o1 ; Cc1ccc(-c2c3ccccc3c(-c3cccc4c3[se]c3cc(-c5c6ccccc6c(-c6ccccc6)c6ccccc56)ccc34)c3ccccc23)s1 ; Cc1cocc1-c1c2ccccc2c(-c2cccc3c2[se]c2cc(-c4c5ccccc5c(-c5ccccc5)c5ccccc45)ccc23)c2ccccc12]

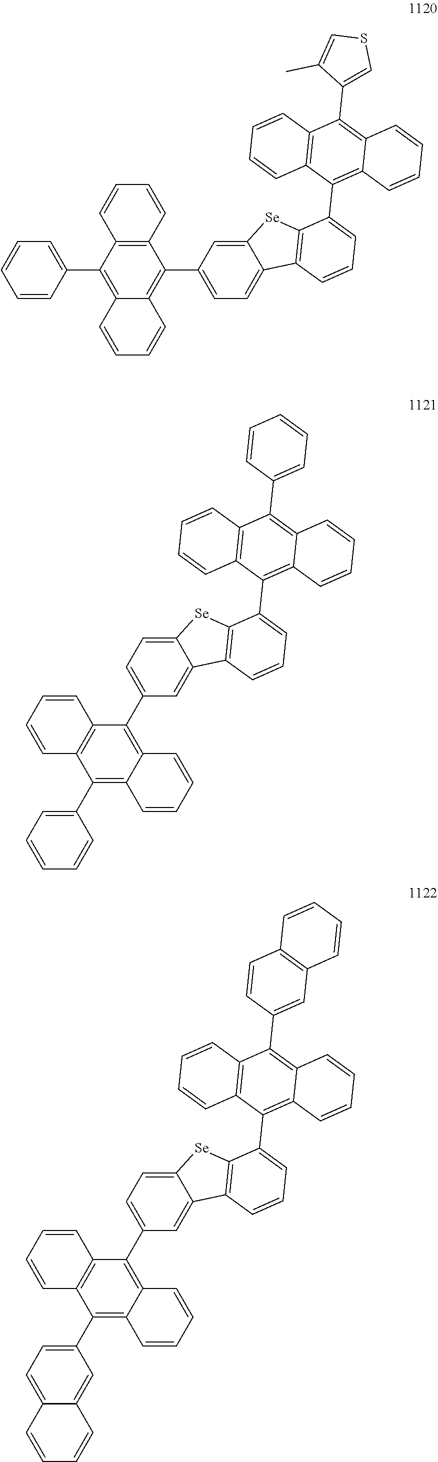 [SMILES: Cc1cscc1-c1c2ccccc2c(-c2cccc3c2[se]c2cc(-c4c5ccccc5c(-c5ccccc5)c5ccccc45)ccc23)c2ccccc12 ; c1ccc(-c2c3ccccc3c(-c3ccc4[se]c5c(-c6c7ccccc7c(-c7ccccc7)c7ccccc67)cccc5c4c3)c3ccccc23)cc1 ; c1ccc2cc(-c3c4ccccc4c(-c4ccc5[se]c6c(-c7c8ccccc8c(-c8ccc9ccccc9c8)c8ccccc78)cccc6c5c4)c4ccccc34)ccc2c1]

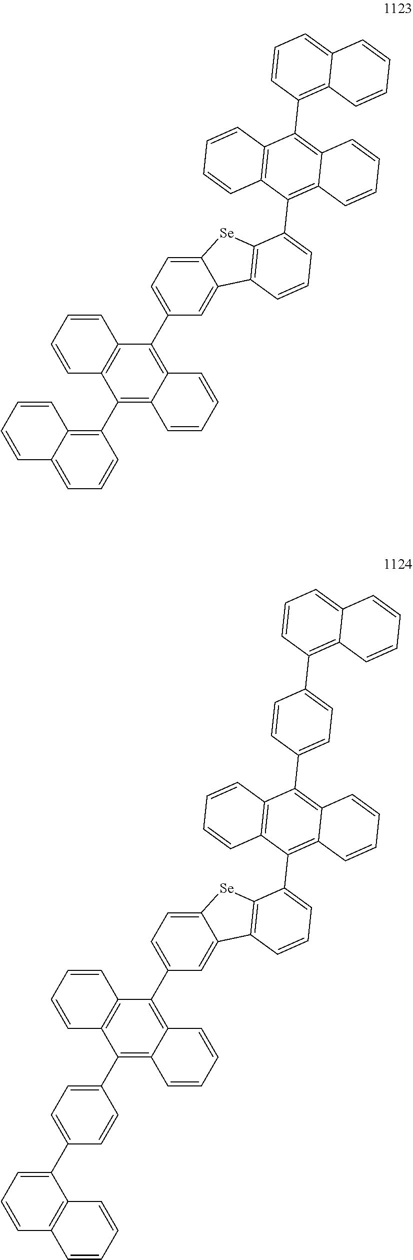 [SMILES: c1ccc2c(-c3c4ccccc4c(-c4ccc5[se]c6c(-c7c8ccccc8c(-c8cccc9ccccc89)c8ccccc78)cccc6c5c4)c4ccccc34)cccc2c1 ; c1ccc2c(-c3ccc(-c4c5ccccc5c(-c5ccc6[se]c7c(-c8c9ccccc9c(-c9ccc(-c%10cccc%11ccccc%10%11)cc9)c9ccccc89)cccc7c6c5)c5ccccc45)cc3)cccc2c1]

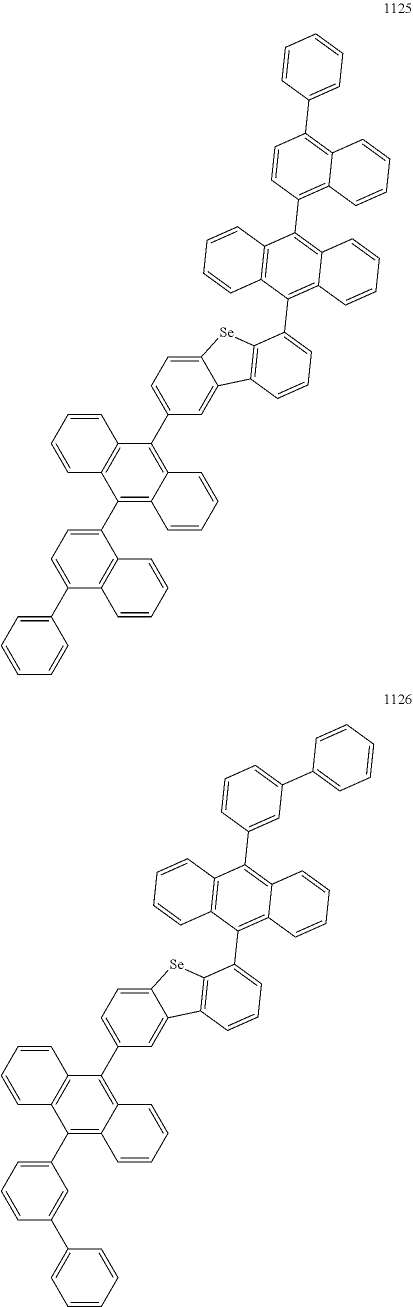 [SMILES: c1ccc(-c2ccc(-c3c4ccccc4c(-c4ccc5[se]c6c(-c7c8ccccc8c(-c8ccc(-c9ccccc9)c9ccccc89)c8ccccc78)cccc6c5c4)c4ccccc34)c3ccccc23)cc1 ; c1ccc(-c2cccc(-c3c4ccccc4c(-c4ccc5[se]c6c(-c7c8ccccc8c(-c8cccc(-c9ccccc9)c8)c8ccccc78)cccc6c5c4)c4ccccc34)c2)cc1]

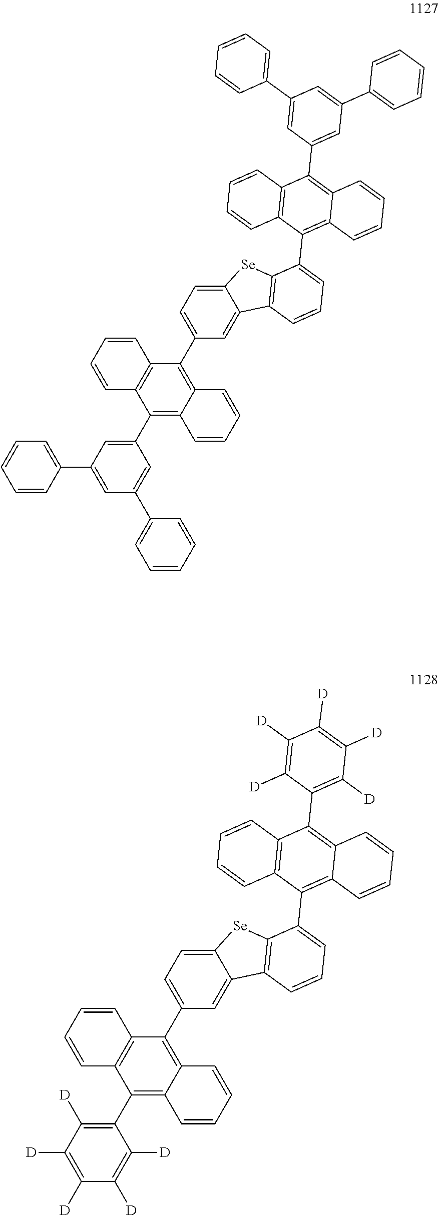 [SMILES: c1ccc(-c2cc(-c3ccccc3)cc(-c3c4ccccc4c(-c4ccc5[se]c6c(-c7c8ccccc8c(-c8cc(-c9ccccc9)cc(-c9ccccc9)c8)c8ccccc78)cccc6c5c4)c4ccccc34)c2)cc1 ; [2H]c1c([2H])c([2H])c(-c2c3ccccc3c(-c3ccc4[se]c5c(-c6c7ccccc7c(-c7c([2H])c([2H])c([2H])c([2H])c7[2H])c7ccccc67)cccc5c4c3)c3ccccc23)c([2H])c1[2H]]

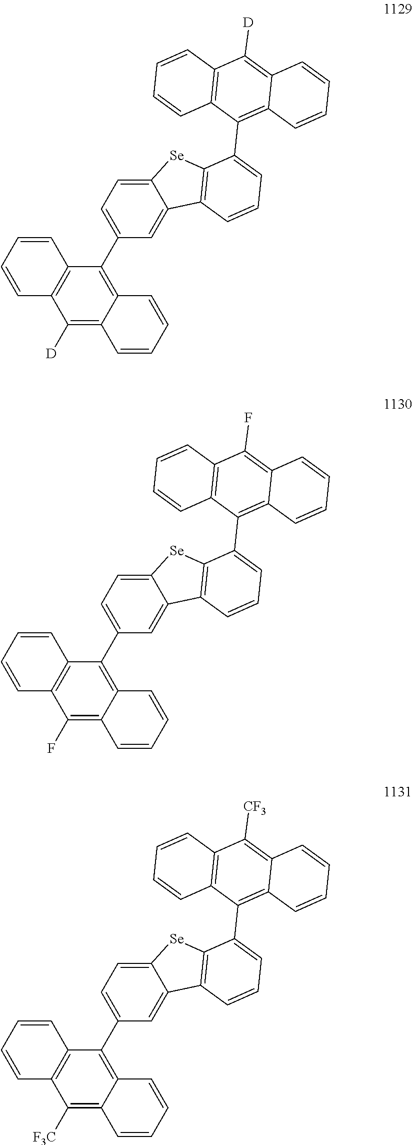 [SMILES: [2H]c1c2ccccc2c(-c2ccc3[se]c4c(-c5c6ccccc6c([2H])c6ccccc56)cccc4c3c2)c2ccccc12 ; Fc1c2ccccc2c(-c2ccc3[se]c4c(-c5c6ccccc6c(F)c6ccccc56)cccc4c3c2)c2ccccc12 ; FC(F)(F)c1c2ccccc2c(-c2ccc3[se]c4c(-c5c6ccccc6c(C(F)(F)F)c6ccccc56)cccc4c3c2)c2ccccc12]

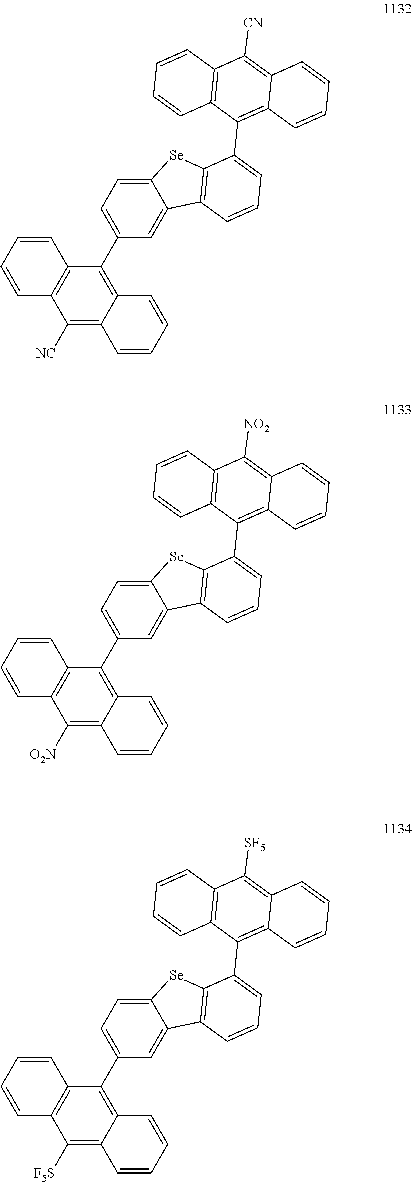

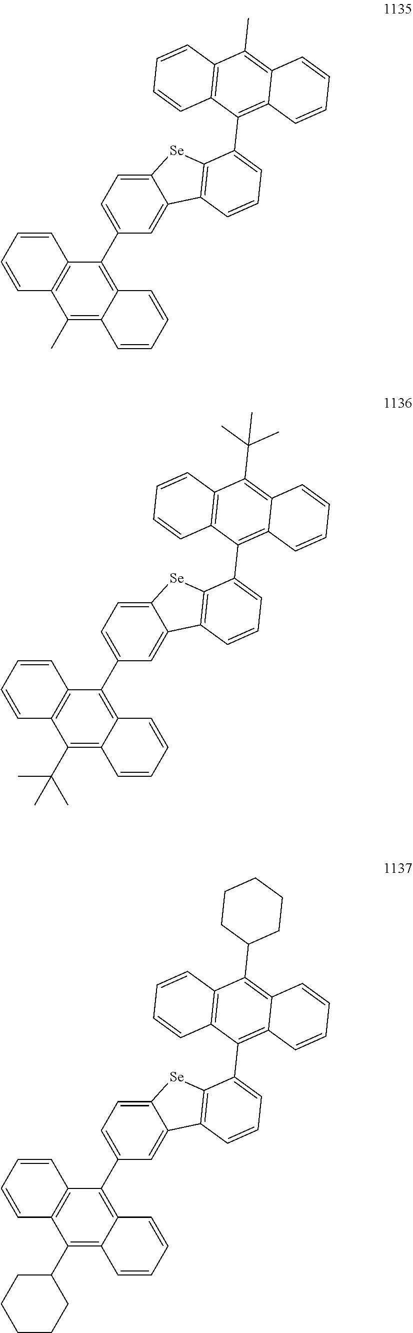 [SMILES: Cc1c2ccccc2c(-c2ccc3[se]c4c(-c5c6ccccc6c(C)c6ccccc56)cccc4c3c2)c2ccccc12 ; CC(C)(C)c1c2ccccc2c(-c2ccc3[se]c4c(-c5c6ccccc6c(C(C)(C)C)c6ccccc56)cccc4c3c2)c2ccccc12 ; c1ccc2c(C3CCCCC3)c3ccccc3c(-c3ccc4[se]c5c(-c6c7ccccc7c(C7CCCCC7)c7ccccc67)cccc5c4c3)c2c1]

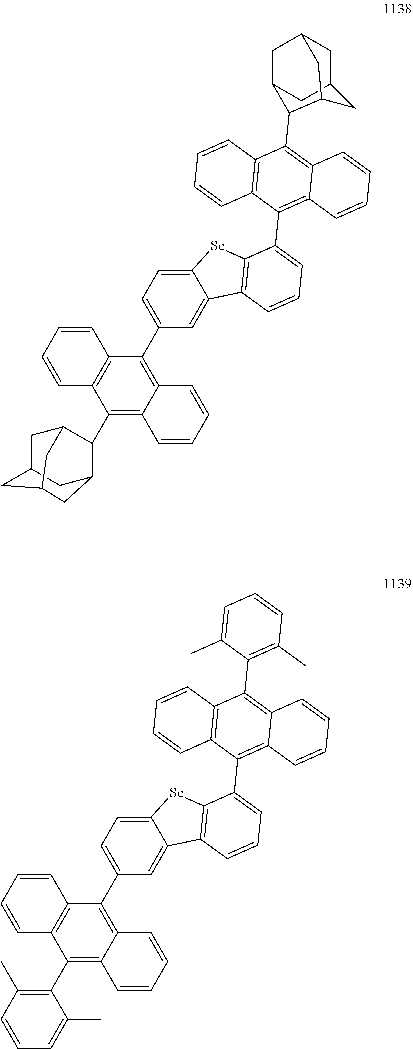 [SMILES: c1ccc2c(C3C4CC5CC(C4)CC3C5)c3ccccc3c(-c3ccc4[se]c5c(-c6c7ccccc7c(C7C8CC9CC(C8)CC7C9)c7ccccc67)cccc5c4c3)c2c1 ; Cc1cccc(C)c1-c1c2ccccc2c(-c2ccc3[se]c4c(-c5c6ccccc6c(-c6c(C)cccc6C)c6ccccc56)cccc4c3c2)c2ccccc12]

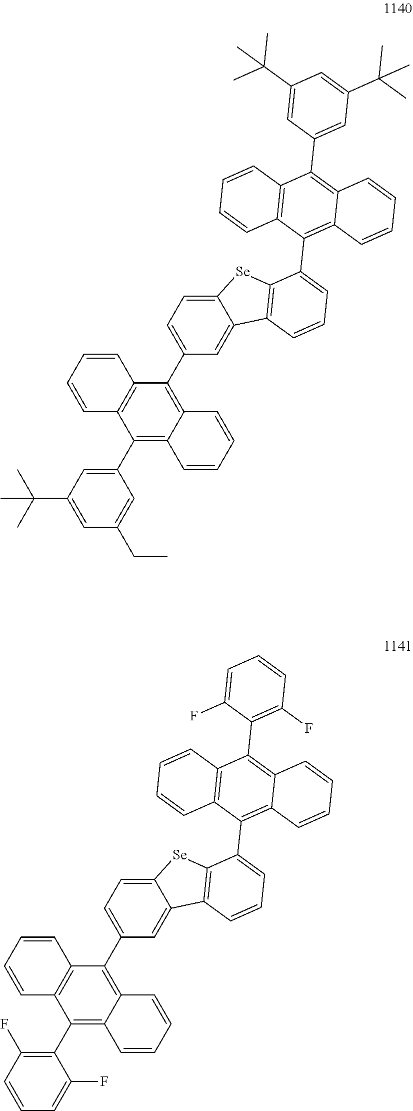 [SMILES: CCc1cc(-c2c3ccccc3c(-c3ccc4[se]c5c(-c6c7ccccc7c(-c7cc(C(C)(C)C)cc(C(C)(C)C)c7)c7ccccc67)cccc5c4c3)c3ccccc23)cc(C(C)(C)C)c1 ; Fc1cccc(F)c1-c1c2ccccc2c(-c2ccc3[se]c4c(-c5c6ccccc6c(-c6c(F)cccc6F)c6ccccc56)cccc4c3c2)c2ccccc12]

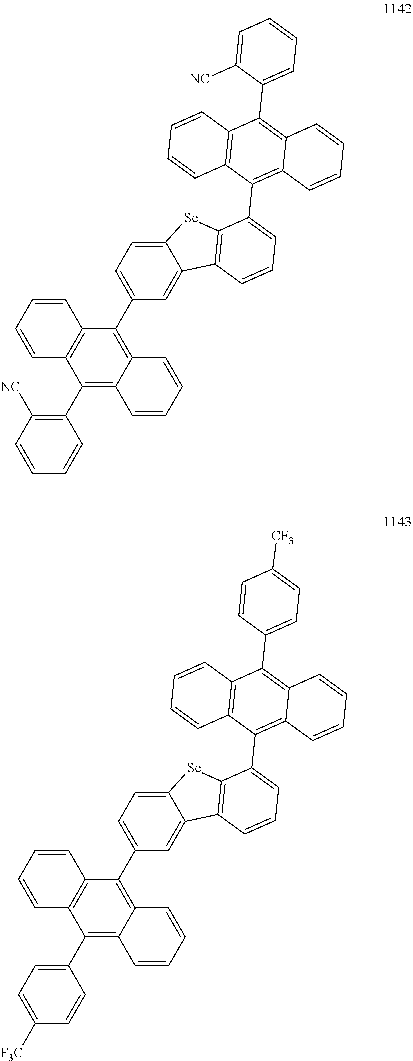 [SMILES: N#Cc1ccccc1-c1c2ccccc2c(-c2ccc3[se]c4c(-c5c6ccccc6c(-c6ccccc6C#N)c6ccccc56)cccc4c3c2)c2ccccc12 ; FC(F)(F)c1ccc(-c2c3ccccc3c(-c3ccc4[se]c5c(-c6c7ccccc7c(-c7ccc(C(F)(F)F)cc7)c7ccccc67)cccc5c4c3)c3ccccc23)cc1]

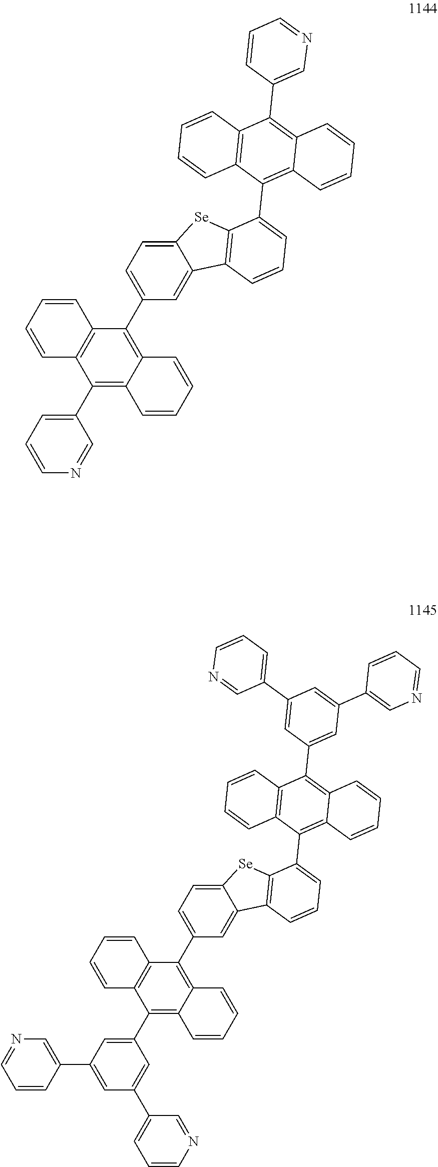 [SMILES: c1cncc(-c2c3ccccc3c(-c3ccc4[se]c5c(-c6c7ccccc7c(-c7cccnc7)c7ccccc67)cccc5c4c3)c3ccccc23)c1 ; c1cncc(-c2cc(-c3cccnc3)cc(-c3c4ccccc4c(-c4ccc5[se]c6c(-c7c8ccccc8c(-c8cc(-c9cccnc9)cc(-c9cccnc9)c8)c8ccccc78)cccc6c5c4)c4ccccc34)c2)c1]

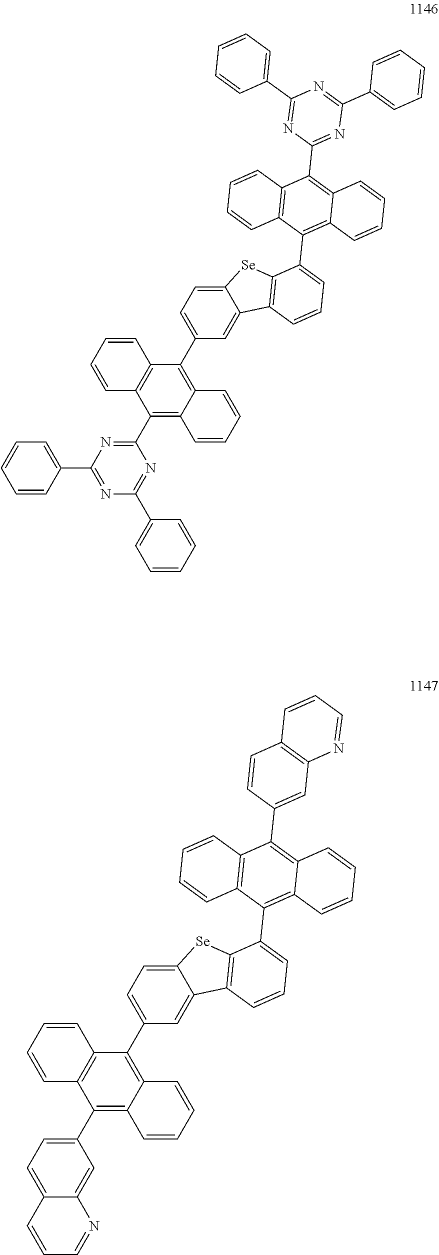 [SMILES: c1ccc(-c2nc(-c3ccccc3)nc(-c3c4ccccc4c(-c4ccc5[se]c6c(-c7c8ccccc8c(-c8nc(-c9ccccc9)nc(-c9ccccc9)n8)c8ccccc78)cccc6c5c4)c4ccccc34)n2)cc1 ; c1cnc2cc(-c3c4ccccc4c(-c4ccc5[se]c6c(-c7c8ccccc8c(-c8ccc9cccnc9c8)c8ccccc78)cccc6c5c4)c4ccccc34)ccc2c1]

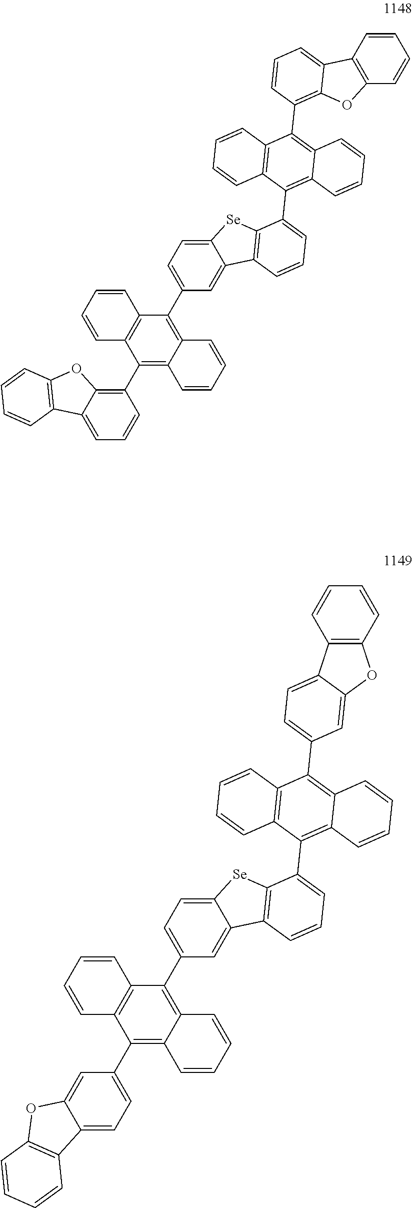 [SMILES: c1ccc2c(c1)oc1c(-c3c4ccccc4c(-c4ccc5[se]c6c(-c7c8ccccc8c(-c8cccc9c8oc8ccccc89)c8ccccc78)cccc6c5c4)c4ccccc34)cccc12 ; c1ccc2c(c1)oc1cc(-c3c4ccccc4c(-c4ccc5[se]c6c(-c7c8ccccc8c(-c8ccc9c(c8)oc8ccccc89)c8ccccc78)cccc6c5c4)c4ccccc34)ccc12]

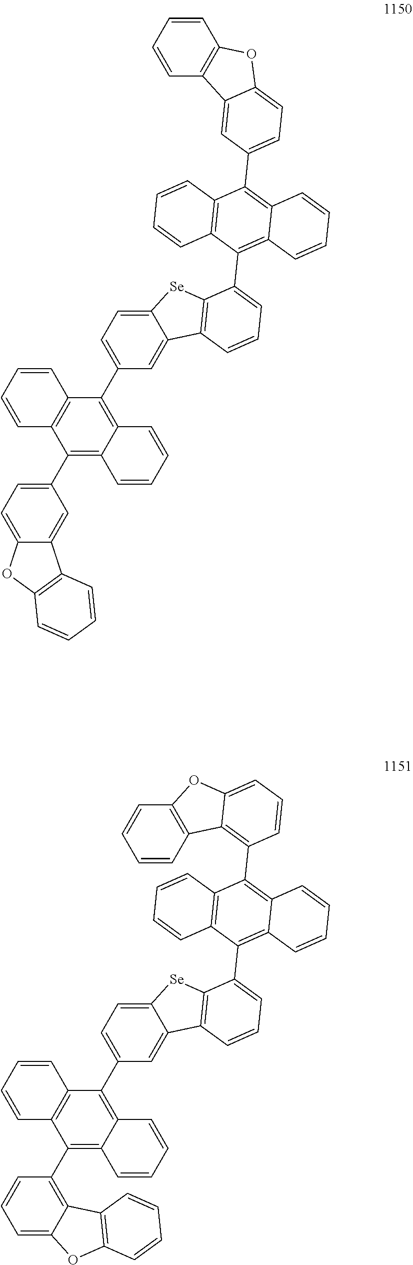 [SMILES: c1ccc2c(c1)oc1ccc(-c3c4ccccc4c(-c4ccc5[se]c6c(-c7c8ccccc8c(-c8ccc9oc%10ccccc%10c9c8)c8ccccc78)cccc6c5c4)c4ccccc34)cc12 ; c1ccc2c(c1)oc1cccc(-c3c4ccccc4c(-c4ccc5[se]c6c(-c7c8ccccc8c(-c8cccc9oc%10ccccc%10c89)c8ccccc78)cccc6c5c4)c4ccccc34)c12]

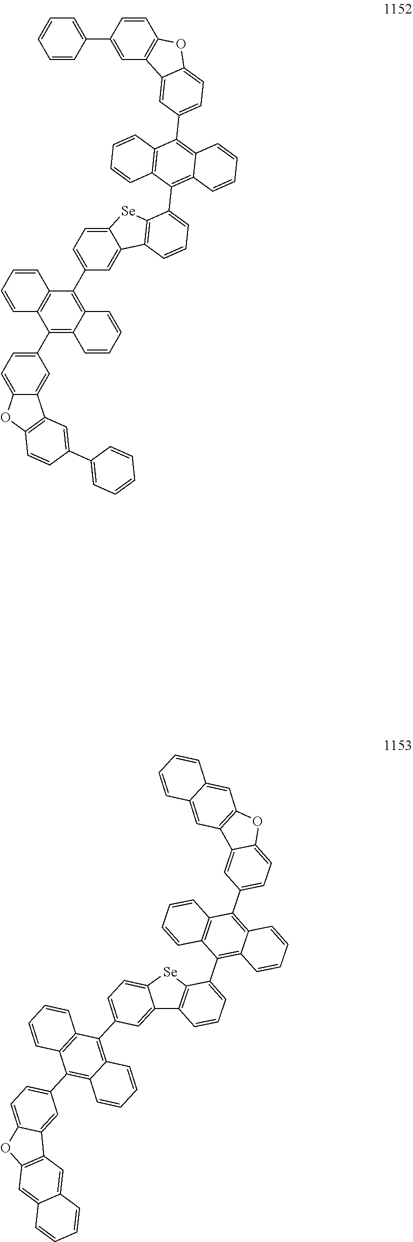 [SMILES: c1ccc(-c2ccc3oc4ccc(-c5c6ccccc6c(-c6ccc7[se]c8c(-c9c%10ccccc%10c(-c%10ccc%11oc%12ccc(-c%13ccccc%13)cc%12c%11c%10)c%10ccccc9%10)cccc8c7c6)c6ccccc56)cc4c3c2)cc1 ; c1ccc2cc3c(cc2c1)oc1ccc(-c2c4ccccc4c(-c4ccc5[se]c6c(-c7c8ccccc8c(-c8ccc9oc%10cc%11ccccc%11cc%10c9c8)c8ccccc78)cccc6c5c4)c4ccccc24)cc13]

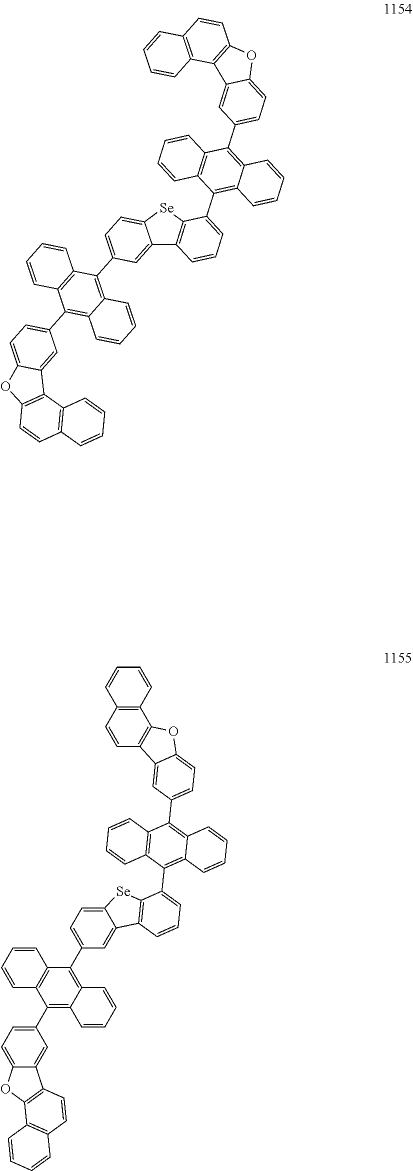 [SMILES: c1ccc2c(c1)ccc1oc3ccc(-c4c5ccccc5c(-c5ccc6[se]c7c(-c8c9ccccc9c(-c9ccc%10oc%11ccc%12ccccc%12c%11c%10c9)c9ccccc89)cccc7c6c5)c5ccccc45)cc3c12 ; c1ccc2c(c1)ccc1c3cc(-c4c5ccccc5c(-c5ccc6[se]c7c(-c8c9ccccc9c(-c9ccc%10oc%11c%12ccccc%12ccc%11c%10c9)c9ccccc89)cccc7c6c5)c5ccccc45)ccc3oc21]

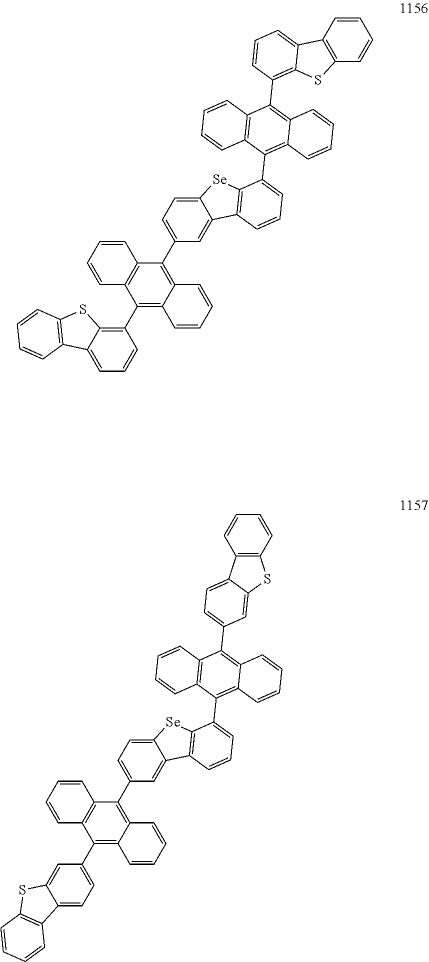 [SMILES: c1ccc2c(c1)sc1c(-c3c4ccccc4c(-c4ccc5[se]c6c(-c7c8ccccc8c(-c8cccc9c8sc8ccccc89)c8ccccc78)cccc6c5c4)c4ccccc34)cccc12 ; c1ccc2c(c1)sc1cc(-c3c4ccccc4c(-c4ccc5[se]c6c(-c7c8ccccc8c(-c8ccc9c(c8)sc8ccccc89)c8ccccc78)cccc6c5c4)c4ccccc34)ccc12]

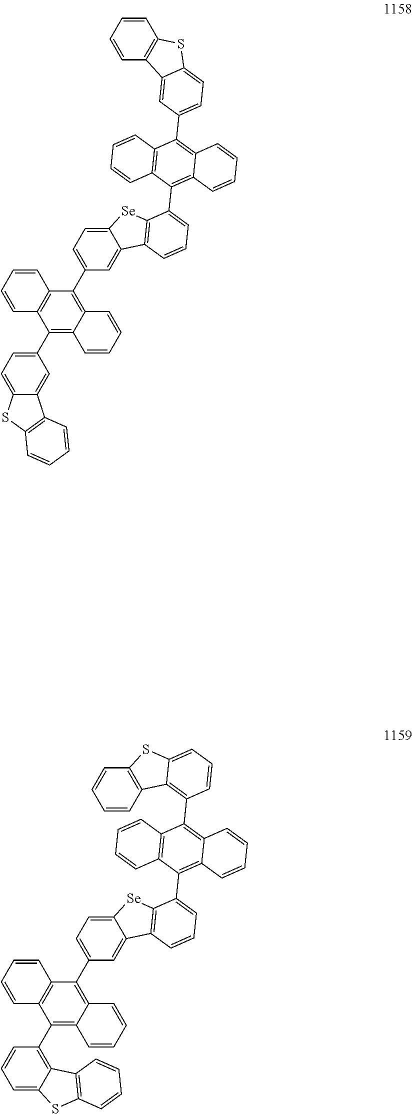 [SMILES: c1ccc2c(c1)sc1ccc(-c3c4ccccc4c(-c4ccc5[se]c6c(-c7c8ccccc8c(-c8ccc9sc%10ccccc%10c9c8)c8ccccc78)cccc6c5c4)c4ccccc34)cc12 ; c1ccc2c(c1)sc1cccc(-c3c4ccccc4c(-c4ccc5[se]c6c(-c7c8ccccc8c(-c8cccc9sc%10ccccc%10c89)c8ccccc78)cccc6c5c4)c4ccccc34)c12]

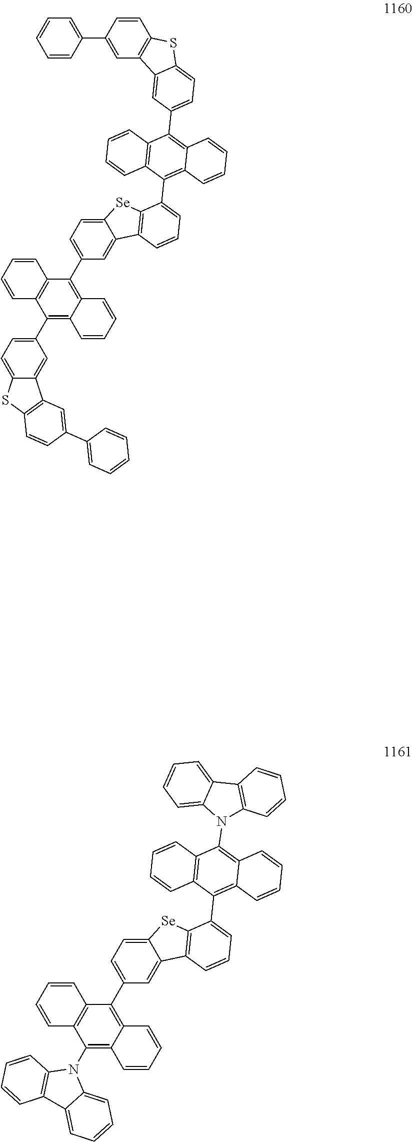 [SMILES: c1ccc(-c2ccc3sc4ccc(-c5c6ccccc6c(-c6ccc7[se]c8c(-c9c%10ccccc%10c(-c%10ccc%11sc%12ccc(-c%13ccccc%13)cc%12c%11c%10)c%10ccccc9%10)cccc8c7c6)c6ccccc56)cc4c3c2)cc1 ; c1ccc2c(-n3c4ccccc4c4ccccc43)c3ccccc3c(-c3ccc4[se]c5c(-c6c7ccccc7c(-n7c8ccccc8c8ccccc87)c7ccccc67)cccc5c4c3)c2c1]

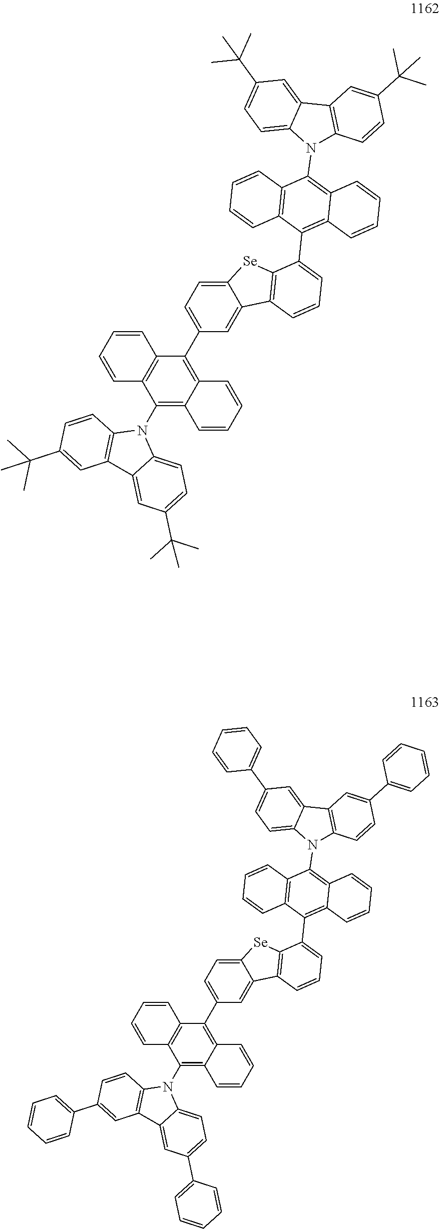 [SMILES: CC(C)(C)c1ccc2c(c1)c1cc(C(C)(C)C)ccc1n2-c1c2ccccc2c(-c2ccc3[se]c4c(-c5c6ccccc6c(-n6c7ccc(C(C)(C)C)cc7c7cc(C(C)(C)C)ccc76)c6ccccc56)cccc4c3c2)c2ccccc12 ; c1ccc(-c2ccc3c(c2)c2cc(-c4ccccc4)ccc2n3-c2c3ccccc3c(-c3ccc4[se]c5c(-c6c7ccccc7c(-n7c8ccc(-c9ccccc9)cc8c8cc(-c9ccccc9)ccc87)c7ccccc67)cccc5c4c3)c3ccccc23)cc1]

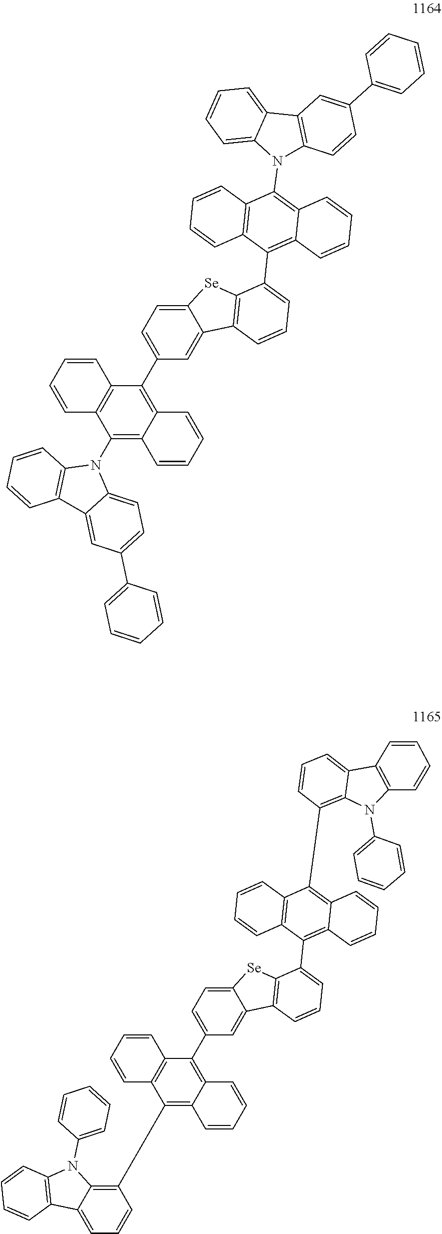 [SMILES: c1ccc(-c2ccc3c(c2)c2ccccc2n3-c2c3ccccc3c(-c3ccc4[se]c5c(-c6c7ccccc7c(-n7c8ccccc8c8cc(-c9ccccc9)ccc87)c7ccccc67)cccc5c4c3)c3ccccc23)cc1 ; c1ccc(-n2c3ccccc3c3cccc(-c4c5ccccc5c(-c5ccc6[se]c7c(-c8c9ccccc9c(-c9cccc%10c%11ccccc%11n(-c%11ccccc%11)c9%10)c9ccccc89)cccc7c6c5)c5ccccc45)c32)cc1]

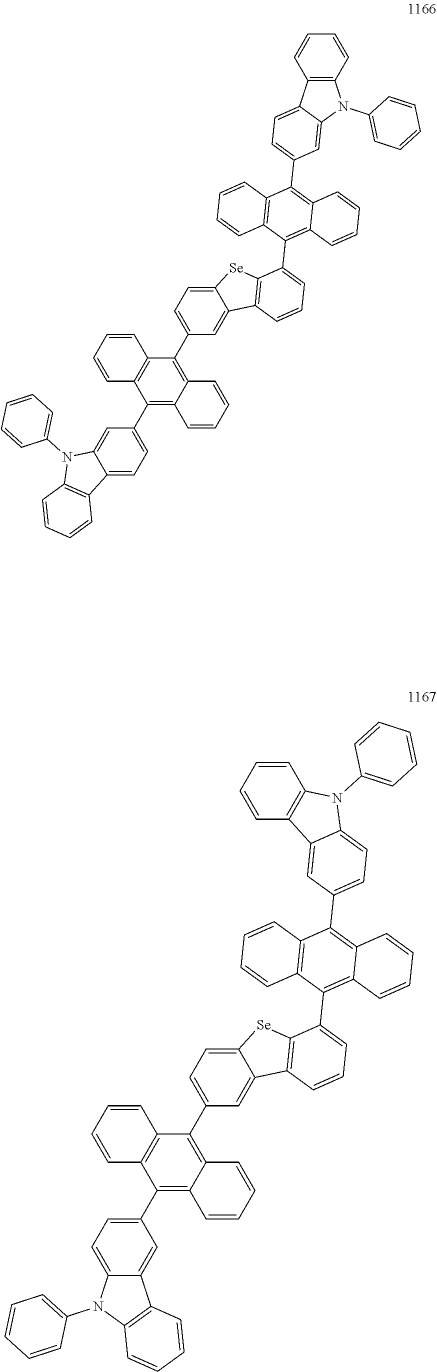 [SMILES: c1ccc(-n2c3ccccc3c3ccc(-c4c5ccccc5c(-c5ccc6[se]c7c(-c8c9ccccc9c(-c9ccc%10c%11ccccc%11n(-c%11ccccc%11)c%10c9)c9ccccc89)cccc7c6c5)c5ccccc45)cc32)cc1 ; c1ccc(-n2c3ccccc3c3cc(-c4c5ccccc5c(-c5ccc6[se]c7c(-c8c9ccccc9c(-c9ccc%10c(c9)c9ccccc9n%10-c9ccccc9)c9ccccc89)cccc7c6c5)c5ccccc45)ccc32)cc1]

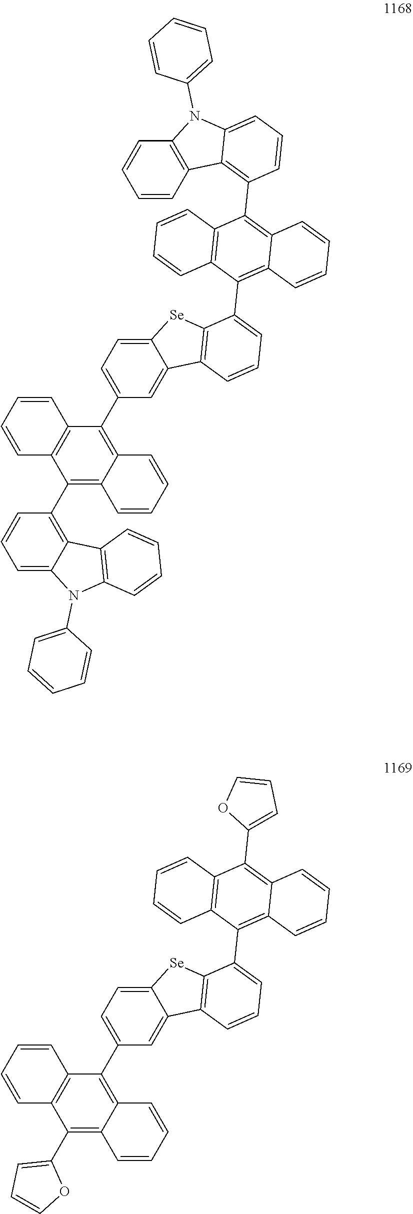 [SMILES: c1ccc(-n2c3ccccc3c3c(-c4c5ccccc5c(-c5ccc6[se]c7c(-c8c9ccccc9c(-c9cccc%10c9c9ccccc9n%10-c9ccccc9)c9ccccc89)cccc7c6c5)c5ccccc45)cccc32)cc1 ; c1coc(-c2c3ccccc3c(-c3ccc4[se]c5c(-c6c7ccccc7c(-c7ccco7)c7ccccc67)cccc5c4c3)c3ccccc23)c1]

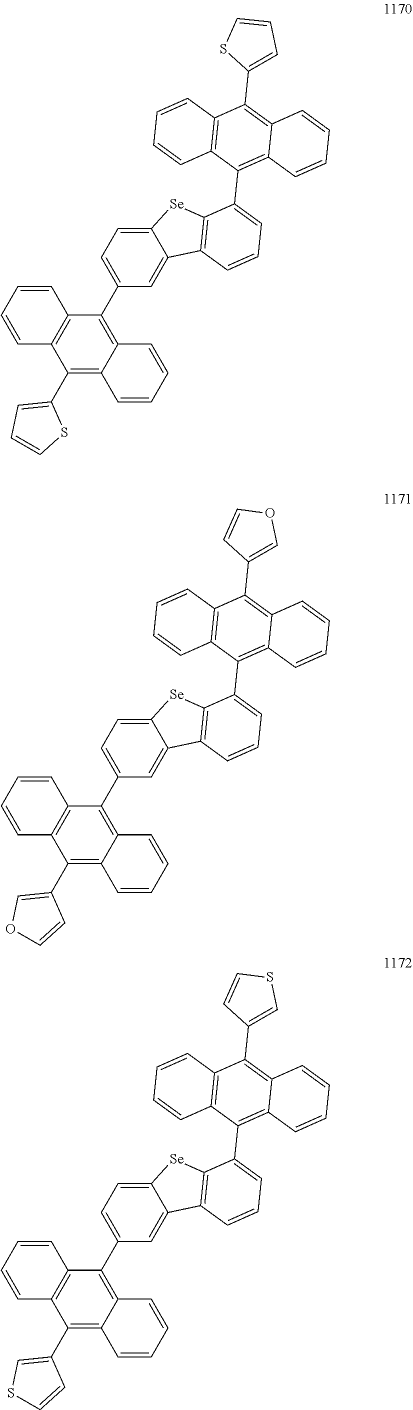 [SMILES: c1csc(-c2c3ccccc3c(-c3ccc4[se]c5c(-c6c7ccccc7c(-c7cccs7)c7ccccc67)cccc5c4c3)c3ccccc23)c1 ; c1ccc2c(-c3ccc4[se]c5c(-c6c7ccccc7c(-c7ccoc7)c7ccccc67)cccc5c4c3)c3ccccc3c(-c3ccoc3)c2c1 ; c1ccc2c(-c3ccc4[se]c5c(-c6c7ccccc7c(-c7ccsc7)c7ccccc67)cccc5c4c3)c3ccccc3c(-c3ccsc3)c2c1]

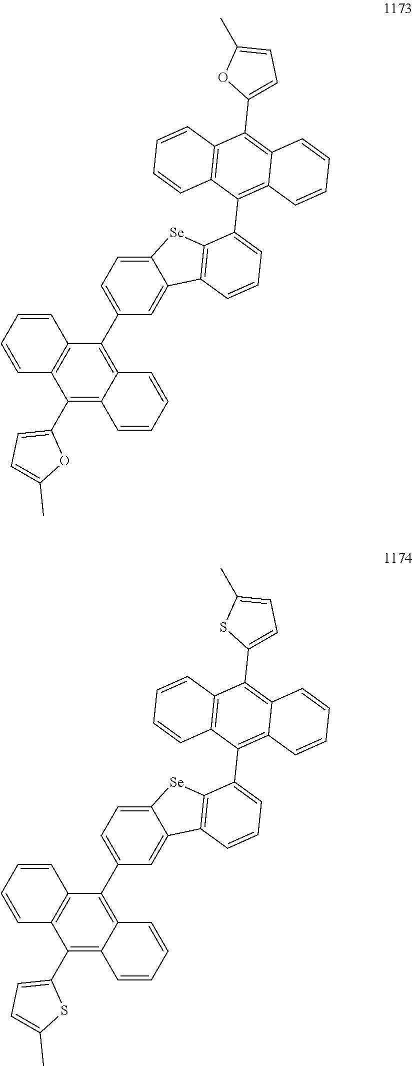 [SMILES: Cc1ccc(-c2c3ccccc3c(-c3ccc4[se]c5c(-c6c7ccccc7c(-c7ccc(C)o7)c7ccccc67)cccc5c4c3)c3ccccc23)o1 ; Cc1ccc(-c2c3ccccc3c(-c3ccc4[se]c5c(-c6c7ccccc7c(-c7ccc(C)s7)c7ccccc67)cccc5c4c3)c3ccccc23)s1]

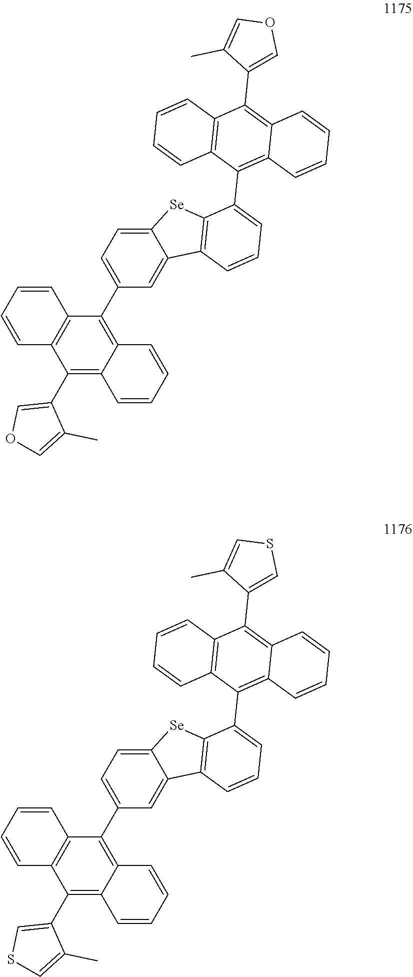 [SMILES: Cc1cocc1-c1c2ccccc2c(-c2ccc3[se]c4c(-c5c6ccccc6c(-c6cocc6C)c6ccccc56)cccc4c3c2)c2ccccc12 ; Cc1cscc1-c1c2ccccc2c(-c2ccc3[se]c4c(-c5c6ccccc6c(-c6cscc6C)c6ccccc56)cccc4c3c2)c2ccccc12]

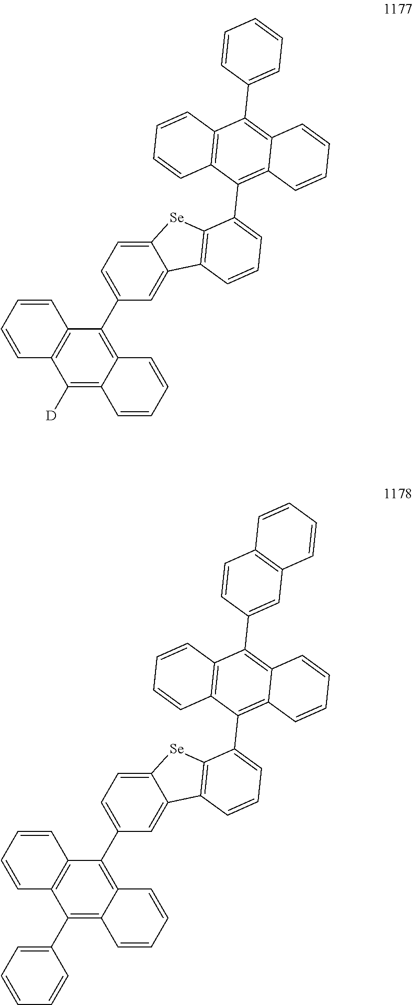 [SMILES: [2H]c1c2ccccc2c(-c2ccc3[se]c4c(-c5c6ccccc6c(-c6ccccc6)c6ccccc56)cccc4c3c2)c2ccccc12 ; c1ccc(-c2c3ccccc3c(-c3ccc4[se]c5c(-c6c7ccccc7c(-c7ccc8ccccc8c7)c7ccccc67)cccc5c4c3)c3ccccc23)cc1]

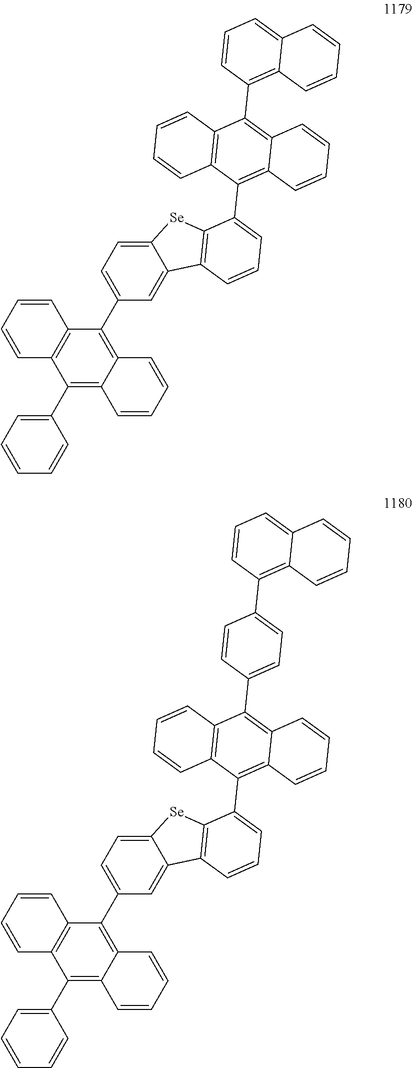 [SMILES: c1ccc(-c2c3ccccc3c(-c3ccc4[se]c5c(-c6c7ccccc7c(-c7cccc8ccccc78)c7ccccc67)cccc5c4c3)c3ccccc23)cc1 ; c1ccc(-c2c3ccccc3c(-c3ccc4[se]c5c(-c6c7ccccc7c(-c7ccc(-c8cccc9ccccc89)cc7)c7ccccc67)cccc5c4c3)c3ccccc23)cc1]

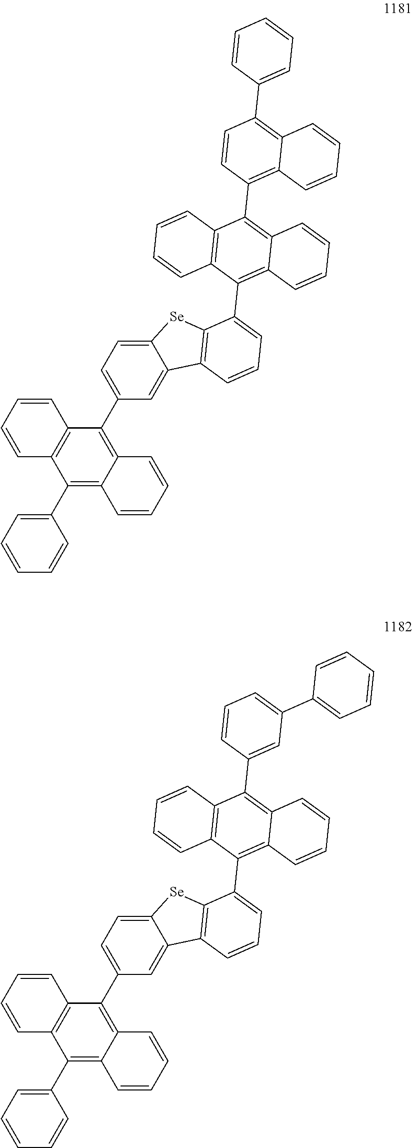 [SMILES: c1ccc(-c2ccc(-c3c4ccccc4c(-c4cccc5c4[se]c4ccc(-c6c7ccccc7c(-c7ccccc7)c7ccccc67)cc45)c4ccccc34)c3ccccc23)cc1 ; c1ccc(-c2cccc(-c3c4ccccc4c(-c4cccc5c4[se]c4ccc(-c6c7ccccc7c(-c7ccccc7)c7ccccc67)cc45)c4ccccc34)c2)cc1]

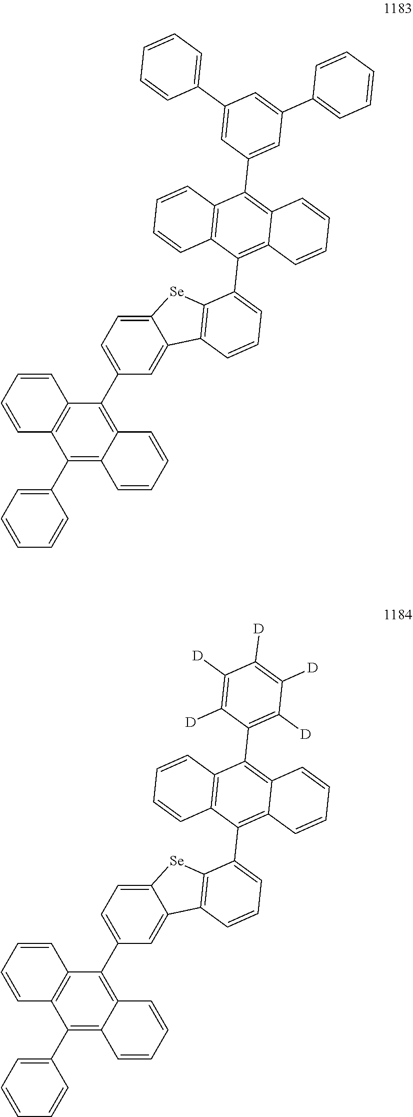 [SMILES: c1ccc(-c2cc(-c3ccccc3)cc(-c3c4ccccc4c(-c4cccc5c4[se]c4ccc(-c6c7ccccc7c(-c7ccccc7)c7ccccc67)cc45)c4ccccc34)c2)cc1 ; [2H]c1c([2H])c([2H])c(-c2c3ccccc3c(-c3cccc4c3[se]c3ccc(-c5c6ccccc6c(-c6ccccc6)c6ccccc56)cc34)c3ccccc23)c([2H])c1[2H]]

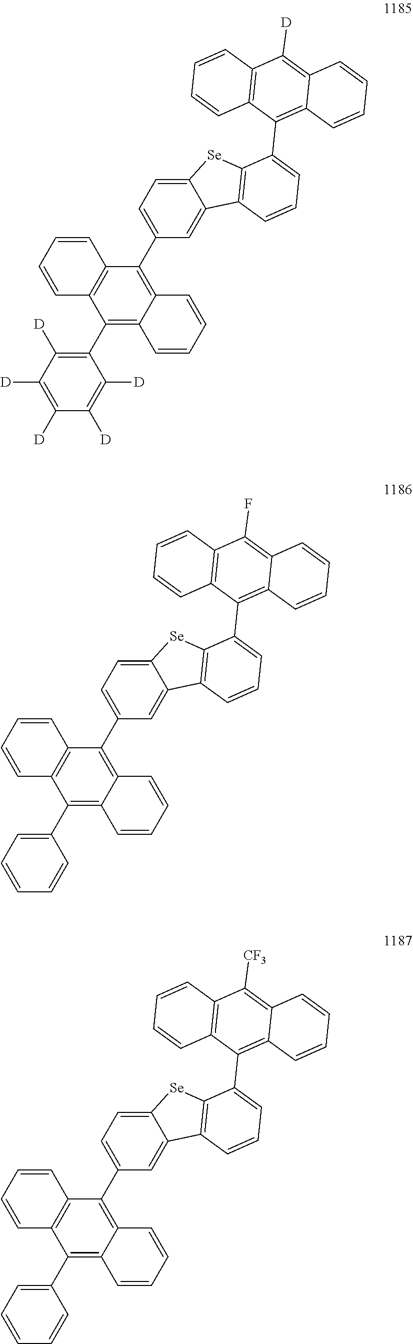 [SMILES: [2H]c1c([2H])c([2H])c(-c2c3ccccc3c(-c3ccc4[se]c5c(-c6c7ccccc7c([2H])c7ccccc67)cccc5c4c3)c3ccccc23)c([2H])c1[2H] ; Fc1c2ccccc2c(-c2cccc3c2[se]c2ccc(-c4c5ccccc5c(-c5ccccc5)c5ccccc45)cc23)c2ccccc12 ; FC(F)(F)c1c2ccccc2c(-c2cccc3c2[se]c2ccc(-c4c5ccccc5c(-c5ccccc5)c5ccccc45)cc23)c2ccccc12]

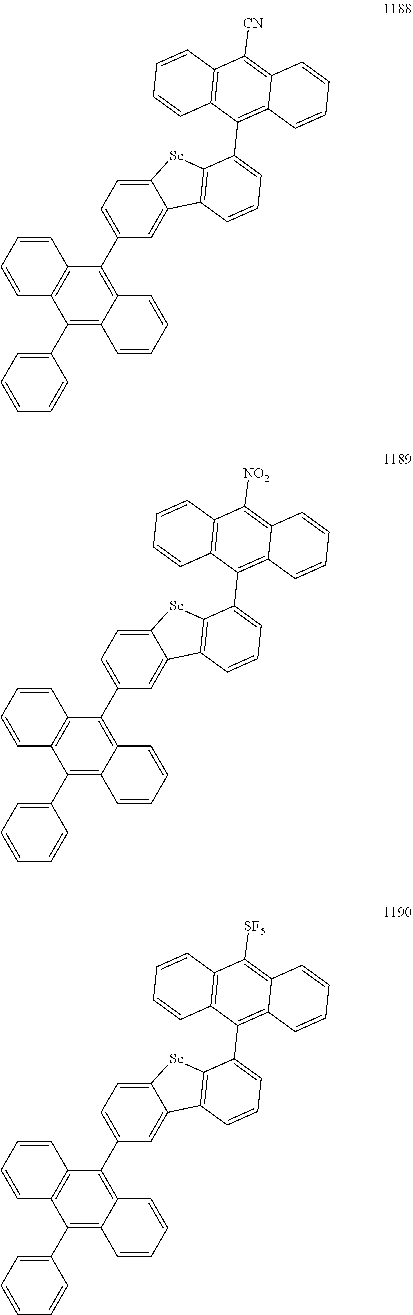

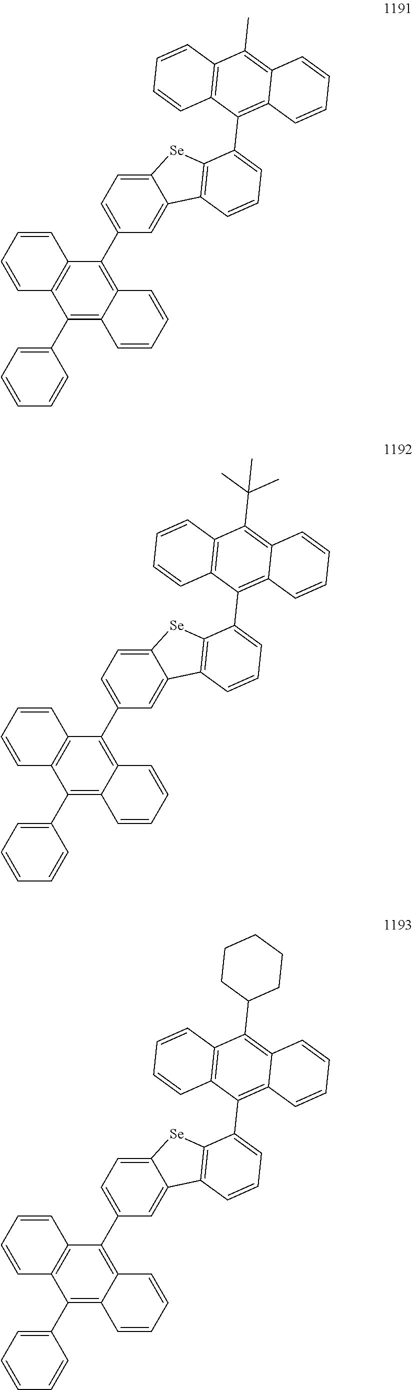 [SMILES: Cc1c2ccccc2c(-c2cccc3c2[se]c2ccc(-c4c5ccccc5c(-c5ccccc5)c5ccccc45)cc23)c2ccccc12 ; CC(C)(C)c1c2ccccc2c(-c2cccc3c2[se]c2ccc(-c4c5ccccc5c(-c5ccccc5)c5ccccc45)cc23)c2ccccc12 ; c1ccc(-c2c3ccccc3c(-c3ccc4[se]c5c(-c6c7ccccc7c(C7CCCCC7)c7ccccc67)cccc5c4c3)c3ccccc23)cc1]

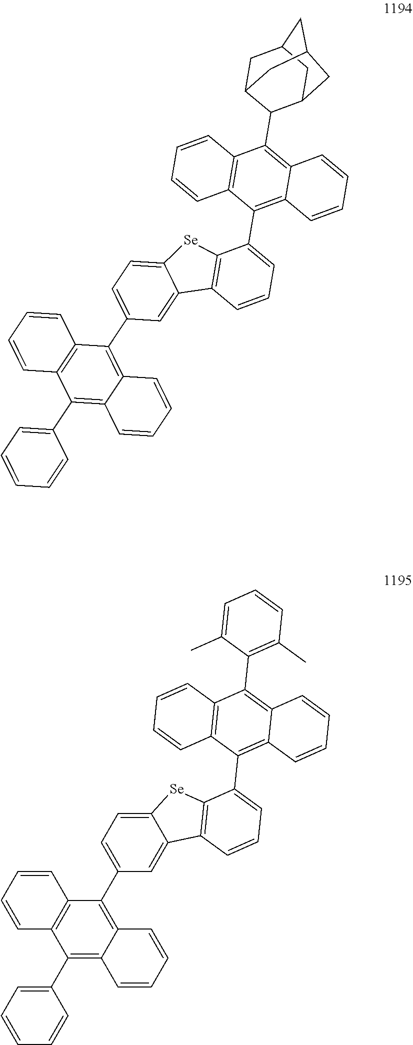 [SMILES: c1ccc(-c2c3ccccc3c(-c3ccc4[se]c5c(-c6c7ccccc7c(C7C8CC9CC(C8)CC7C9)c7ccccc67)cccc5c4c3)c3ccccc23)cc1 ; Cc1cccc(C)c1-c1c2ccccc2c(-c2cccc3c2[se]c2ccc(-c4c5ccccc5c(-c5ccccc5)c5ccccc45)cc23)c2ccccc12]

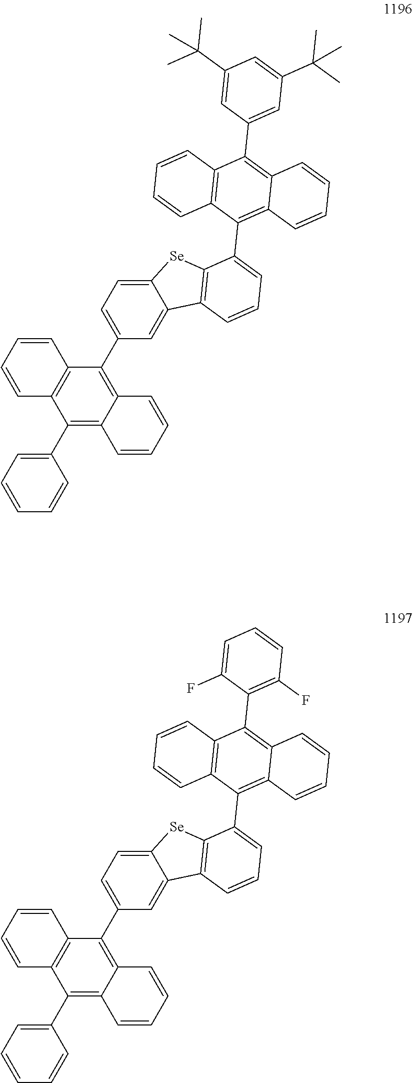 [SMILES: CC(C)(C)c1cc(-c2c3ccccc3c(-c3cccc4c3[se]c3ccc(-c5c6ccccc6c(-c6ccccc6)c6ccccc56)cc34)c3ccccc23)cc(C(C)(C)C)c1 ; Fc1cccc(F)c1-c1c2ccccc2c(-c2cccc3c2[se]c2ccc(-c4c5ccccc5c(-c5ccccc5)c5ccccc45)cc23)c2ccccc12]

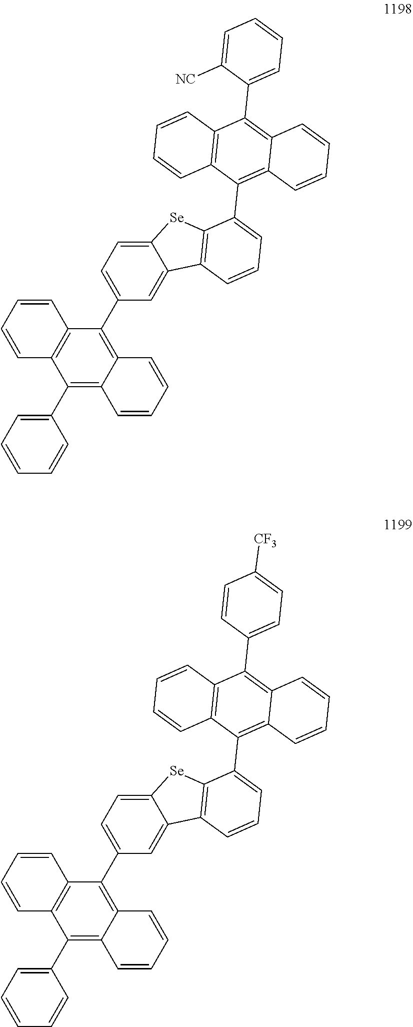 [SMILES: N#Cc1ccccc1-c1c2ccccc2c(-c2cccc3c2[se]c2ccc(-c4c5ccccc5c(-c5ccccc5)c5ccccc45)cc23)c2ccccc12 ; FC(F)(F)c1ccc(-c2c3ccccc3c(-c3cccc4c3[se]c3ccc(-c5c6ccccc6c(-c6ccccc6)c6ccccc56)cc34)c3ccccc23)cc1]

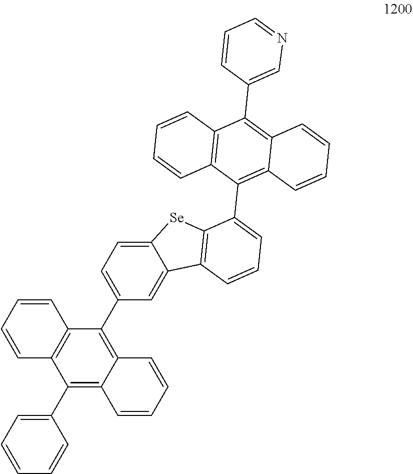 [SMILES: c1ccc(-c2c3ccccc3c(-c3ccc4[se]c5c(-c6c7ccccc7c(-c7cccnc7)c7ccccc67)cccc5c4c3)c3ccccc23)cc1]

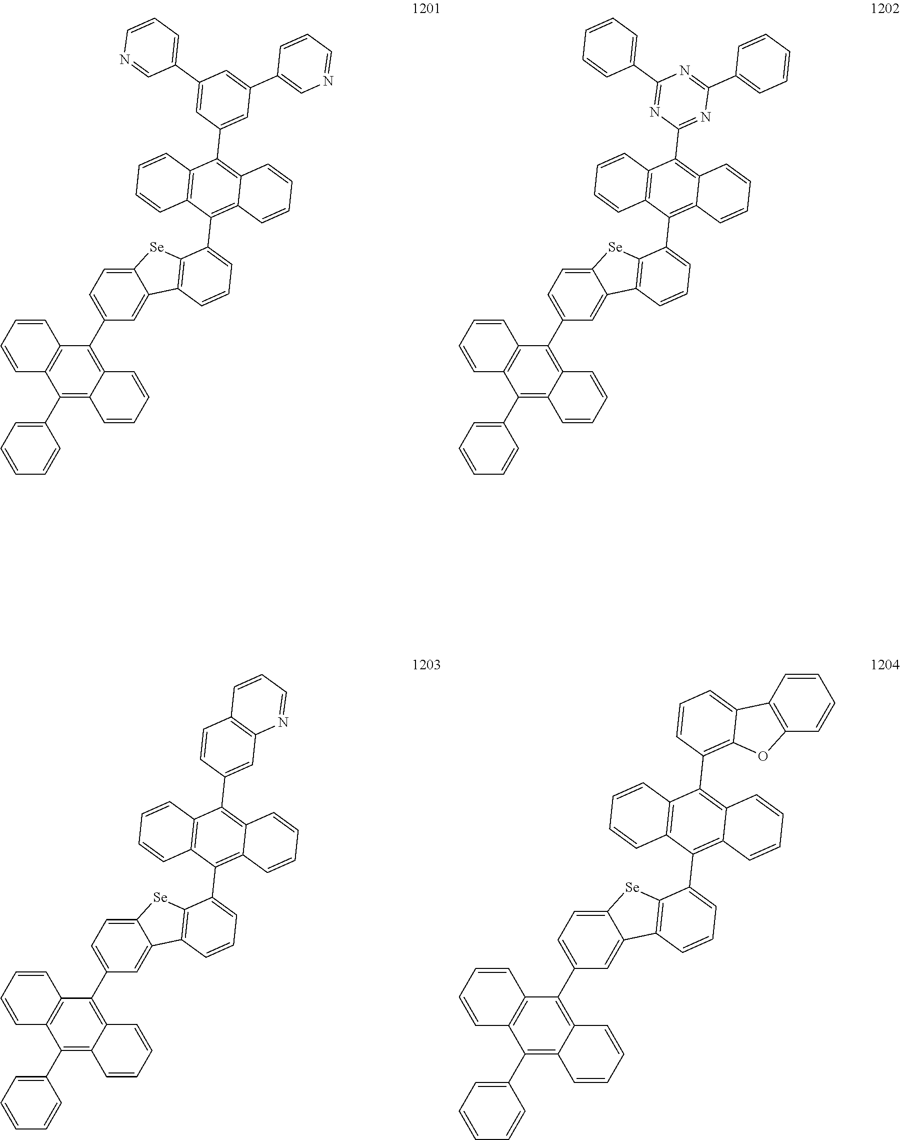 [SMILES: c1ccc(-c2c3ccccc3c(-c3ccc4[se]c5c(-c6c7ccccc7c(-c7cc(-c8cccnc8)cc(-c8cccnc8)c7)c7ccccc67)cccc5c4c3)c3ccccc23)cc1 ; c1ccc(-c2nc(-c3ccccc3)nc(-c3c4ccccc4c(-c4cccc5c4[se]c4ccc(-c6c7ccccc7c(-c7ccccc7)c7ccccc67)cc45)c4ccccc34)n2)cc1 ; c1ccc(-c2c3ccccc3c(-c3ccc4[se]c5c(-c6c7ccccc7c(-c7ccc8cccnc8c7)c7ccccc67)cccc5c4c3)c3ccccc23)cc1 ; c1ccc(-c2c3ccccc3c(-c3ccc4[se]c5c(-c6c7ccccc7c(-c7cccc8c7oc7ccccc78)c7ccccc67)cccc5c4c3)c3ccccc23)cc1]

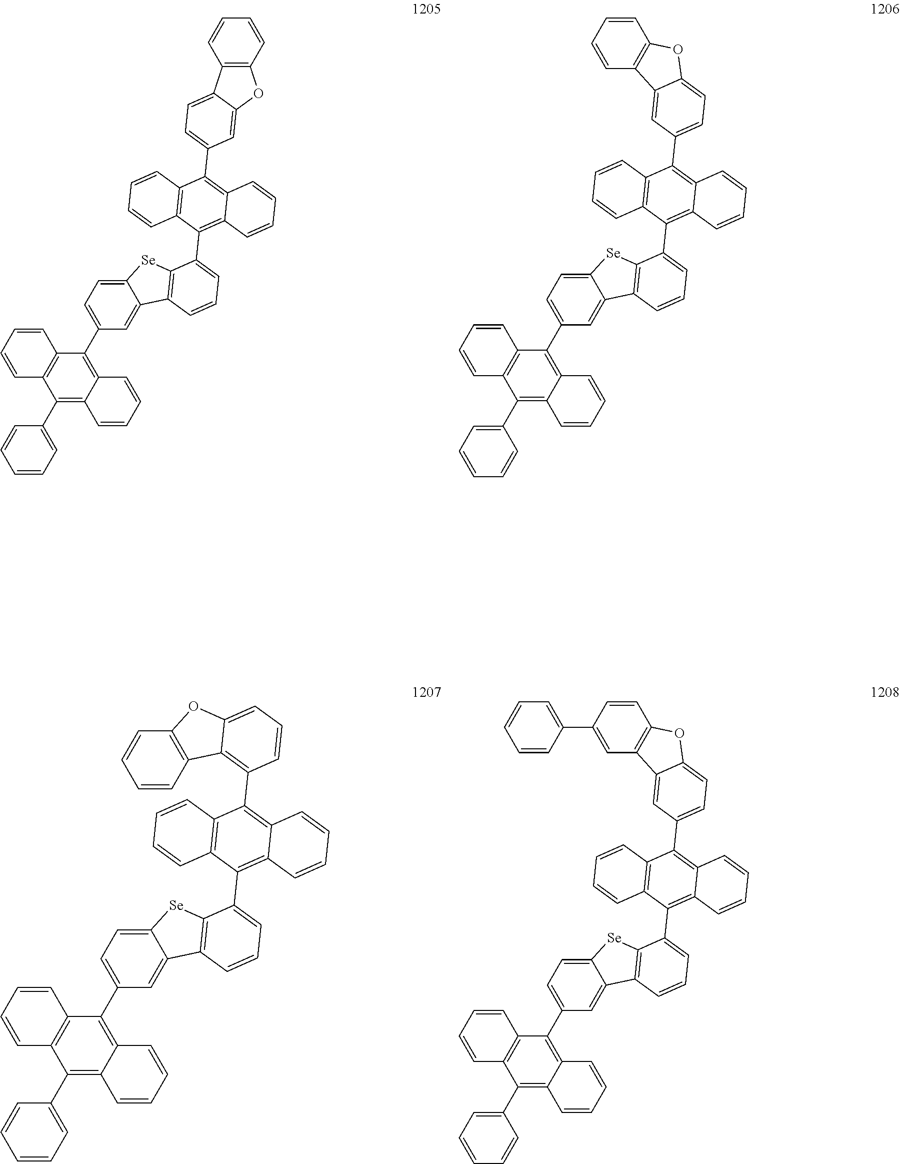 [SMILES: c1ccc(-c2c3ccccc3c(-c3ccc4[se]c5c(-c6c7ccccc7c(-c7ccc8c(c7)oc7ccccc78)c7ccccc67)cccc5c4c3)c3ccccc23)cc1 ; c1ccc(-c2c3ccccc3c(-c3ccc4[se]c5c(-c6c7ccccc7c(-c7ccc8oc9ccccc9c8c7)c7ccccc67)cccc5c4c3)c3ccccc23)cc1 ; c1ccc(-c2c3ccccc3c(-c3ccc4[se]c5c(-c6c7ccccc7c(-c7cccc8oc9ccccc9c78)c7ccccc67)cccc5c4c3)c3ccccc23)cc1 ; c1ccc(-c2ccc3oc4ccc(-c5c6ccccc6c(-c6cccc7c6[se]c6ccc(-c8c9ccccc9c(-c9ccccc9)c9ccccc89)cc67)c6ccccc56)cc4c3c2)cc1]

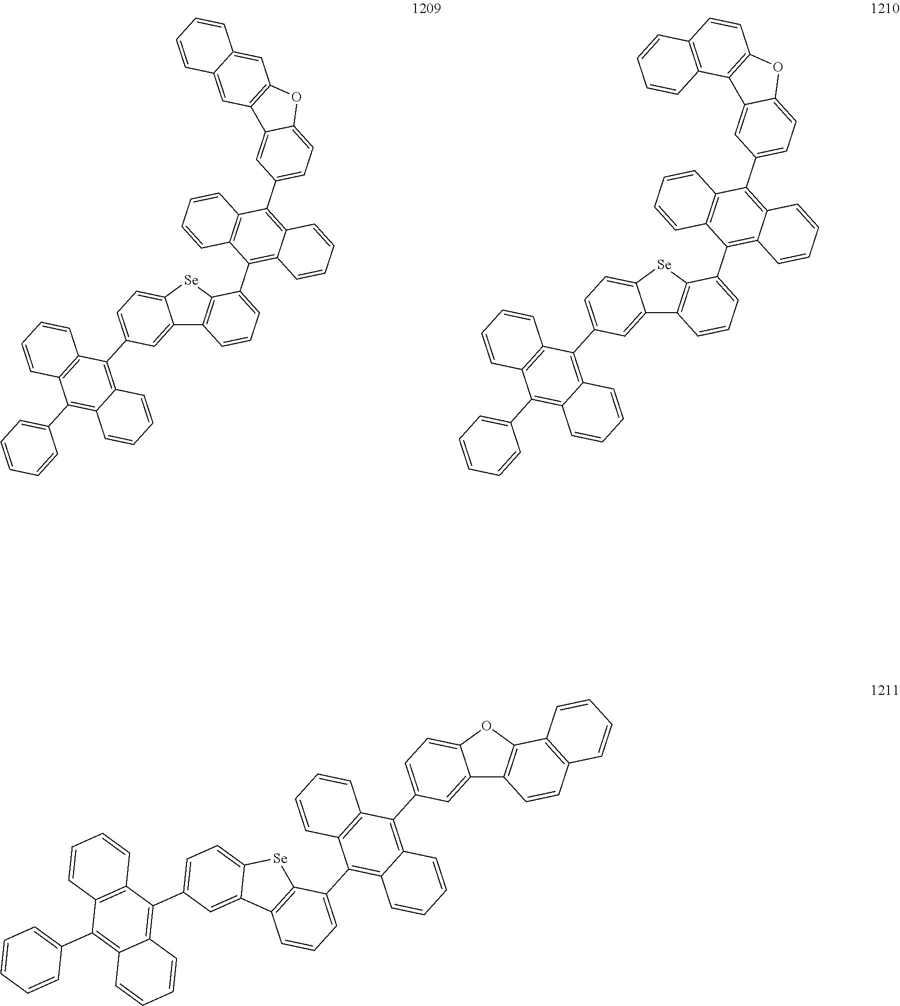 [SMILES: c1ccc(-c2c3ccccc3c(-c3ccc4[se]c5c(-c6c7ccccc7c(-c7ccc8oc9cc%10ccccc%10cc9c8c7)c7ccccc67)cccc5c4c3)c3ccccc23)cc1 ; c1ccc(-c2c3ccccc3c(-c3ccc4[se]c5c(-c6c7ccccc7c(-c7ccc8oc9ccc%10ccccc%10c9c8c7)c7ccccc67)cccc5c4c3)c3ccccc23)cc1 ; c1ccc(-c2c3ccccc3c(-c3ccc4[se]c5c(-c6c7ccccc7c(-c7ccc8oc9c%10ccccc%10ccc9c8c7)c7ccccc67)cccc5c4c3)c3ccccc23)cc1]

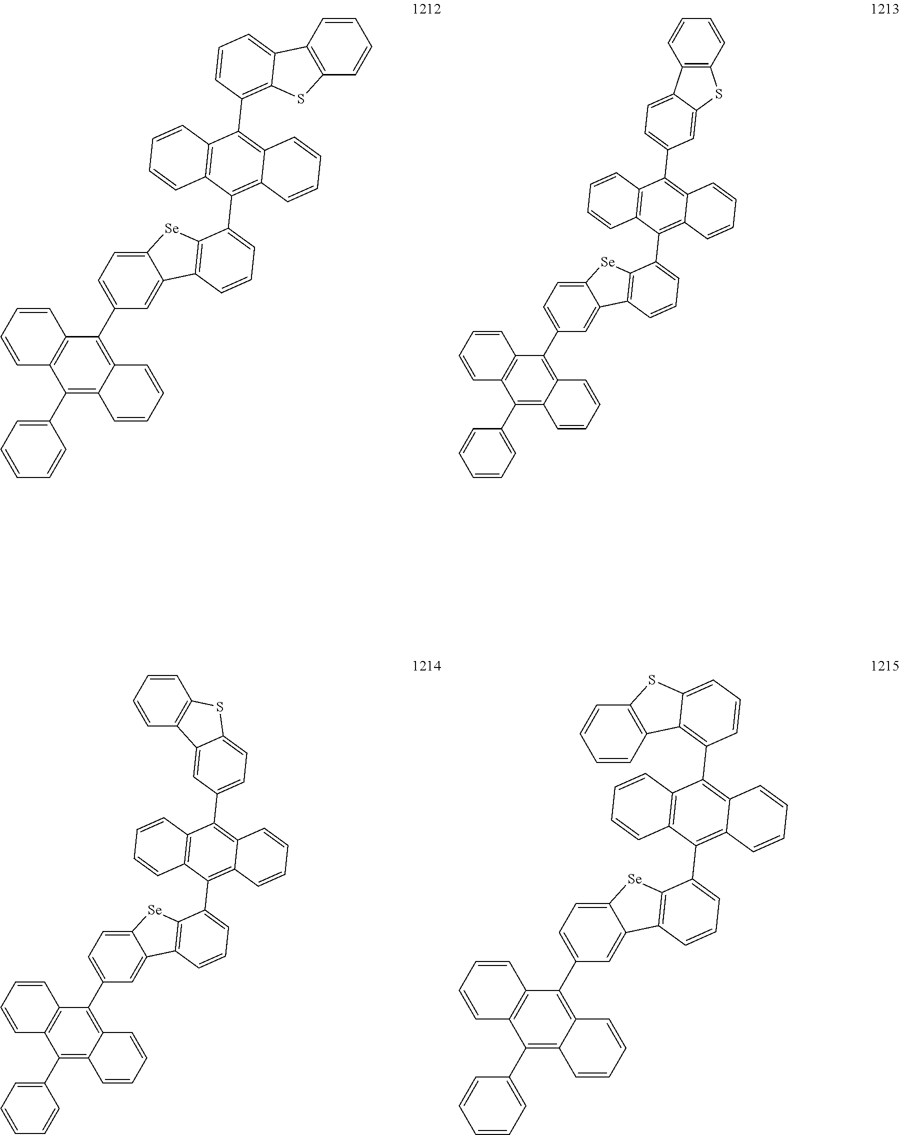 [SMILES: c1ccc(-c2c3ccccc3c(-c3ccc4[se]c5c(-c6c7ccccc7c(-c7cccc8c7sc7ccccc78)c7ccccc67)cccc5c4c3)c3ccccc23)cc1 ; c1ccc(-c2c3ccccc3c(-c3ccc4[se]c5c(-c6c7ccccc7c(-c7ccc8c(c7)sc7ccccc78)c7ccccc67)cccc5c4c3)c3ccccc23)cc1 ; c1ccc(-c2c3ccccc3c(-c3ccc4[se]c5c(-c6c7ccccc7c(-c7ccc8sc9ccccc9c8c7)c7ccccc67)cccc5c4c3)c3ccccc23)cc1 ; c1ccc(-c2c3ccccc3c(-c3ccc4[se]c5c(-c6c7ccccc7c(-c7cccc8sc9ccccc9c78)c7ccccc67)cccc5c4c3)c3ccccc23)cc1]

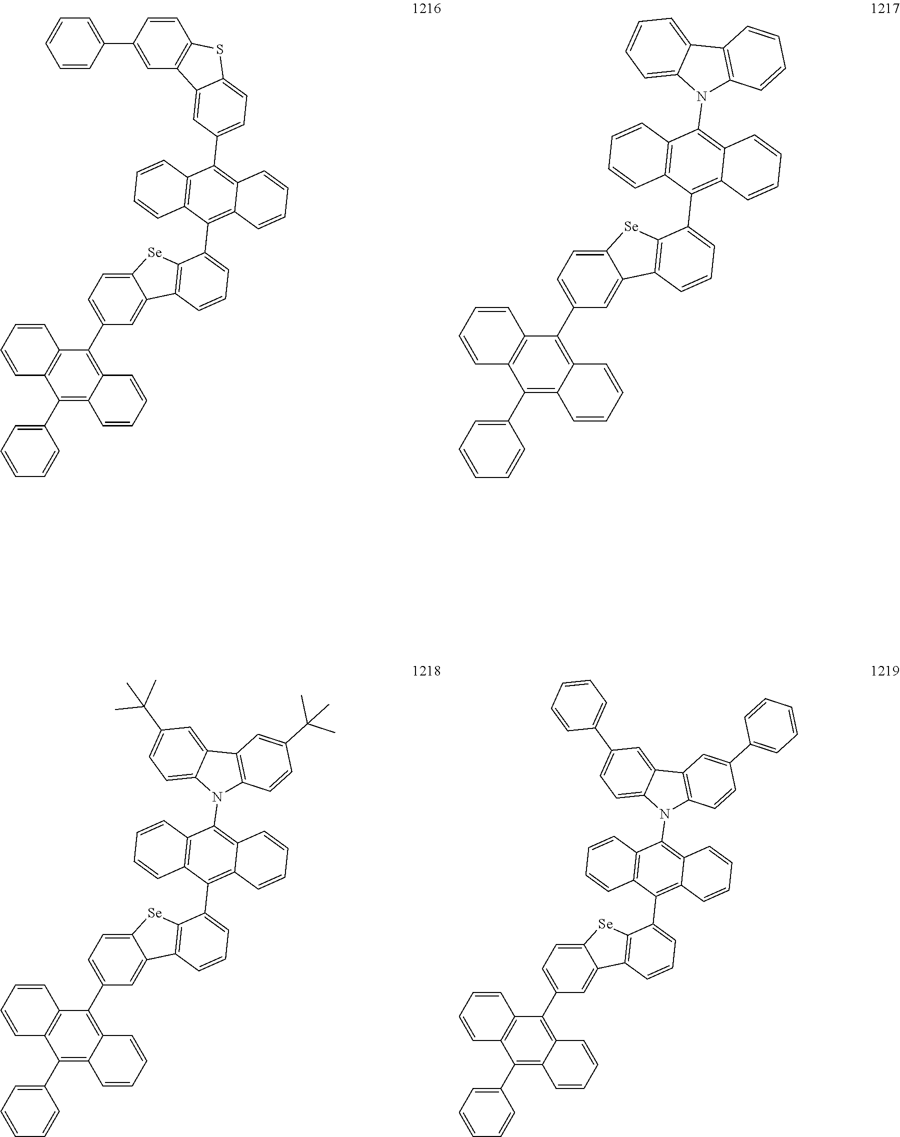 [SMILES: c1ccc(-c2ccc3sc4ccc(-c5c6ccccc6c(-c6cccc7c6[se]c6ccc(-c8c9ccccc9c(-c9ccccc9)c9ccccc89)cc67)c6ccccc56)cc4c3c2)cc1 ; c1ccc(-c2c3ccccc3c(-c3ccc4[se]c5c(-c6c7ccccc7c(-n7c8ccccc8c8ccccc87)c7ccccc67)cccc5c4c3)c3ccccc23)cc1 ; CC(C)(C)c1ccc2c(c1)c1cc(C(C)(C)C)ccc1n2-c1c2ccccc2c(-c2cccc3c2[se]c2ccc(-c4c5ccccc5c(-c5ccccc5)c5ccccc45)cc23)c2ccccc12 ; c1ccc(-c2ccc3c(c2)c2cc(-c4ccccc4)ccc2n3-c2c3ccccc3c(-c3cccc4c3[se]c3ccc(-c5c6ccccc6c(-c6ccccc6)c6ccccc56)cc34)c3ccccc23)cc1]

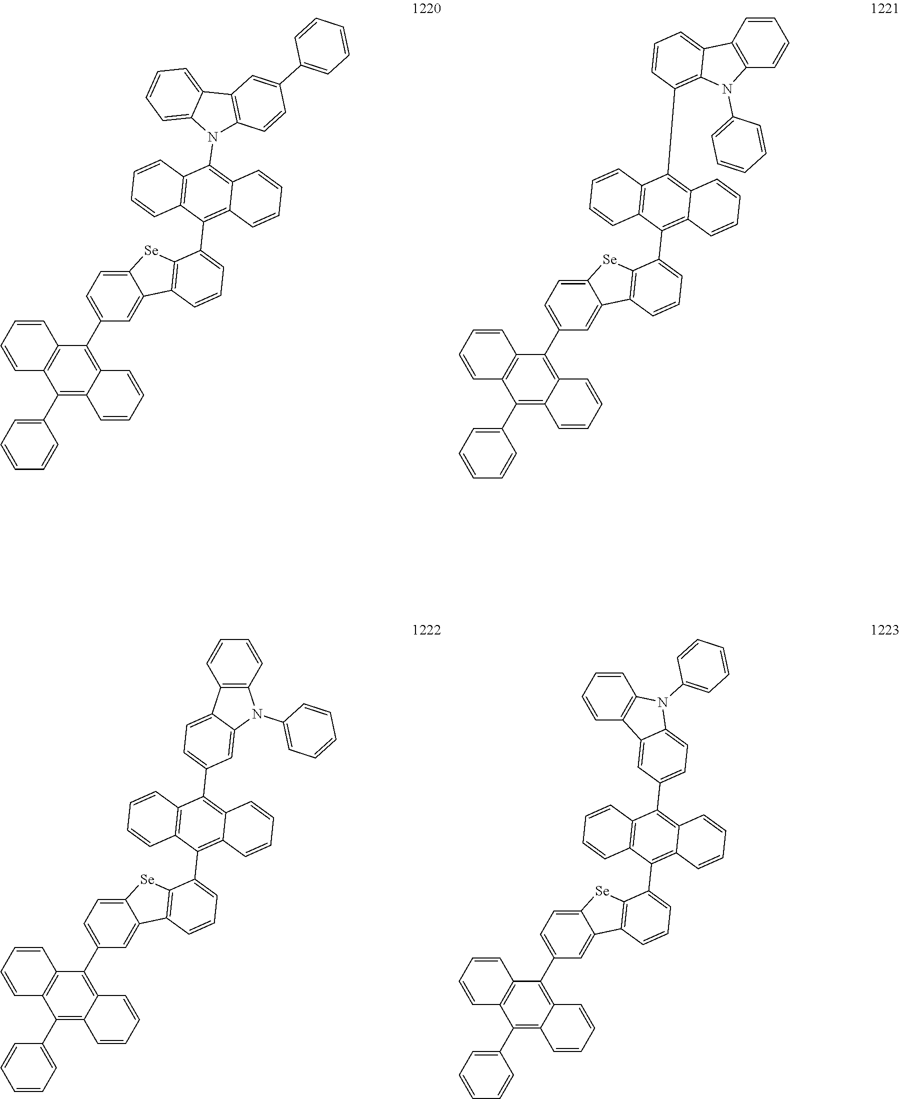 [SMILES: c1ccc(-c2ccc3c(c2)c2ccccc2n3-c2c3ccccc3c(-c3cccc4c3[se]c3ccc(-c5c6ccccc6c(-c6ccccc6)c6ccccc56)cc34)c3ccccc23)cc1 ; c1ccc(-c2c3ccccc3c(-c3ccc4[se]c5c(-c6c7ccccc7c(-c7cccc8c9ccccc9n(-c9ccccc9)c78)c7ccccc67)cccc5c4c3)c3ccccc23)cc1 ; c1ccc(-c2c3ccccc3c(-c3ccc4[se]c5c(-c6c7ccccc7c(-c7ccc8c9ccccc9n(-c9ccccc9)c8c7)c7ccccc67)cccc5c4c3)c3ccccc23)cc1 ; c1ccc(-c2c3ccccc3c(-c3ccc4[se]c5c(-c6c7ccccc7c(-c7ccc8c(c7)c7ccccc7n8-c7ccccc7)c7ccccc67)cccc5c4c3)c3ccccc23)cc1]

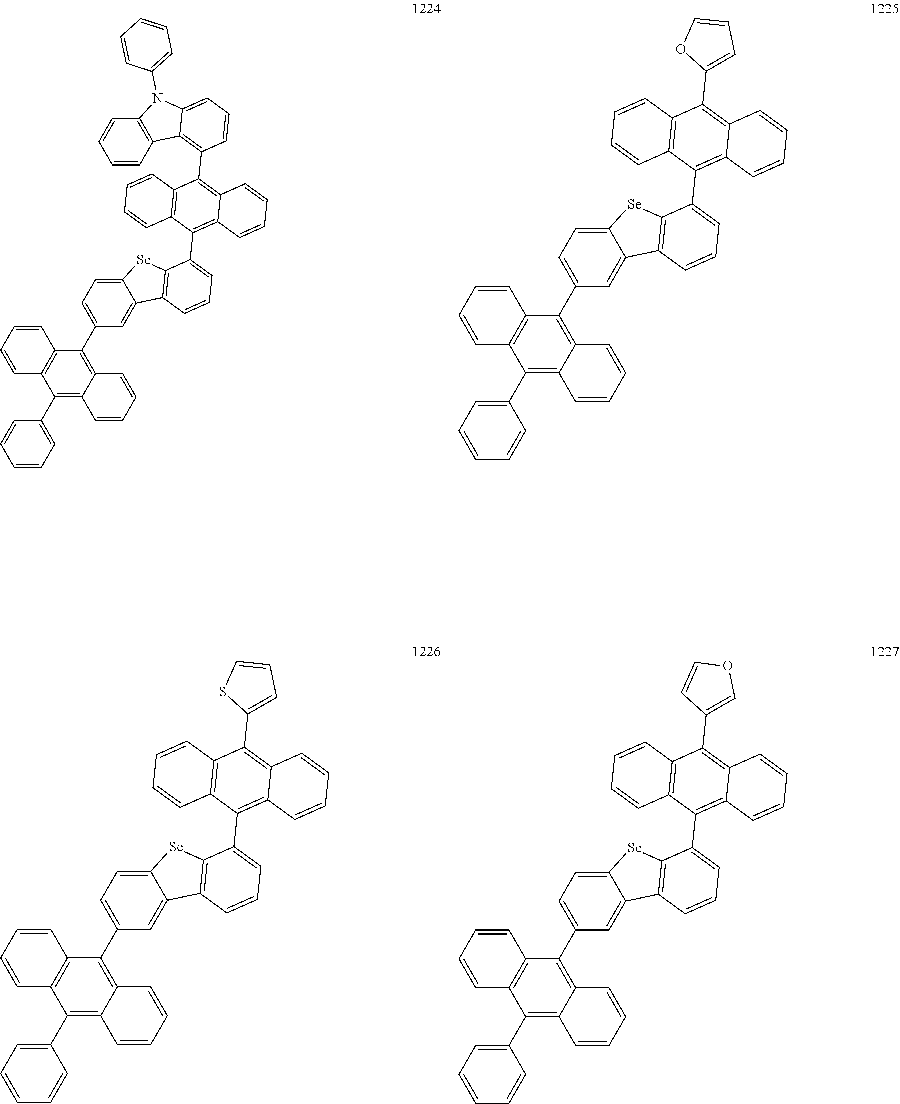 [SMILES: c1ccc(-c2c3ccccc3c(-c3ccc4[se]c5c(-c6c7ccccc7c(-c7cccc8c7c7ccccc7n8-c7ccccc7)c7ccccc67)cccc5c4c3)c3ccccc23)cc1 ; c1ccc(-c2c3ccccc3c(-c3ccc4[se]c5c(-c6c7ccccc7c(-c7ccco7)c7ccccc67)cccc5c4c3)c3ccccc23)cc1 ; c1ccc(-c2c3ccccc3c(-c3ccc4[se]c5c(-c6c7ccccc7c(-c7cccs7)c7ccccc67)cccc5c4c3)c3ccccc23)cc1 ; c1ccc(-c2c3ccccc3c(-c3ccc4[se]c5c(-c6c7ccccc7c(-c7ccoc7)c7ccccc67)cccc5c4c3)c3ccccc23)cc1]

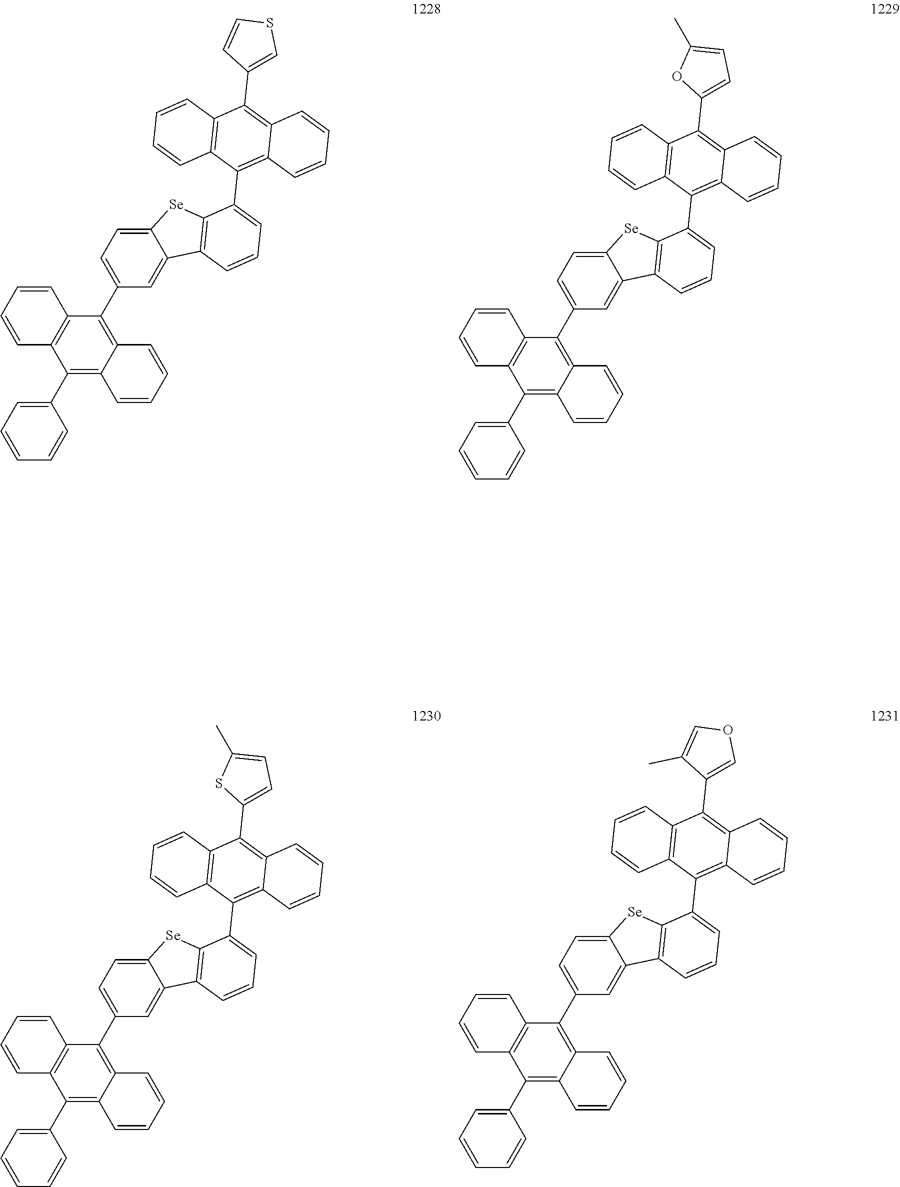 [SMILES: c1ccc(-c2c3ccccc3c(-c3ccc4[se]c5c(-c6c7ccccc7c(-c7ccsc7)c7ccccc67)cccc5c4c3)c3ccccc23)cc1 ; Cc1ccc(-c2c3ccccc3c(-c3cccc4c3[se]c3ccc(-c5c6ccccc6c(-c6ccccc6)c6ccccc56)cc34)c3ccccc23)o1 ; Cc1ccc(-c2c3ccccc3c(-c3cccc4c3[se]c3ccc(-c5c6ccccc6c(-c6ccccc6)c6ccccc56)cc34)c3ccccc23)s1 ; Cc1cocc1-c1c2ccccc2c(-c2cccc3c2[se]c2ccc(-c4c5ccccc5c(-c5ccccc5)c5ccccc45)cc23)c2ccccc12]

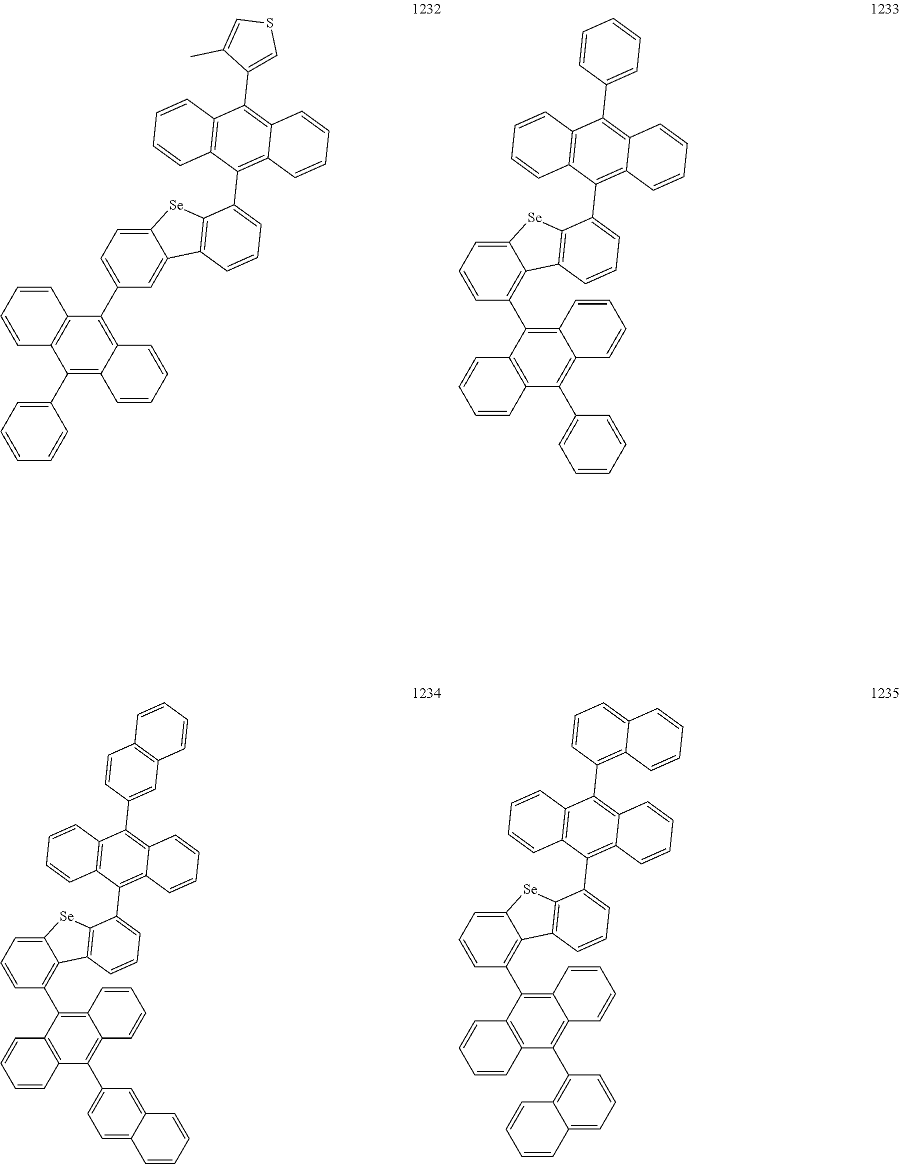 [SMILES: Cc1cscc1-c1c2ccccc2c(-c2cccc3c2[se]c2ccc(-c4c5ccccc5c(-c5ccccc5)c5ccccc45)cc23)c2ccccc12 ; c1ccc(-c2c3ccccc3c(-c3cccc4c3[se]c3cccc(-c5c6ccccc6c(-c6ccccc6)c6ccccc56)c34)c3ccccc23)cc1 ; c1ccc2cc(-c3c4ccccc4c(-c4cccc5c4[se]c4cccc(-c6c7ccccc7c(-c7ccc8ccccc8c7)c7ccccc67)c45)c4ccccc34)ccc2c1 ; c1ccc2c(-c3c4ccccc4c(-c4cccc5c4[se]c4cccc(-c6c7ccccc7c(-c7cccc8ccccc78)c7ccccc67)c45)c4ccccc34)cccc2c1]

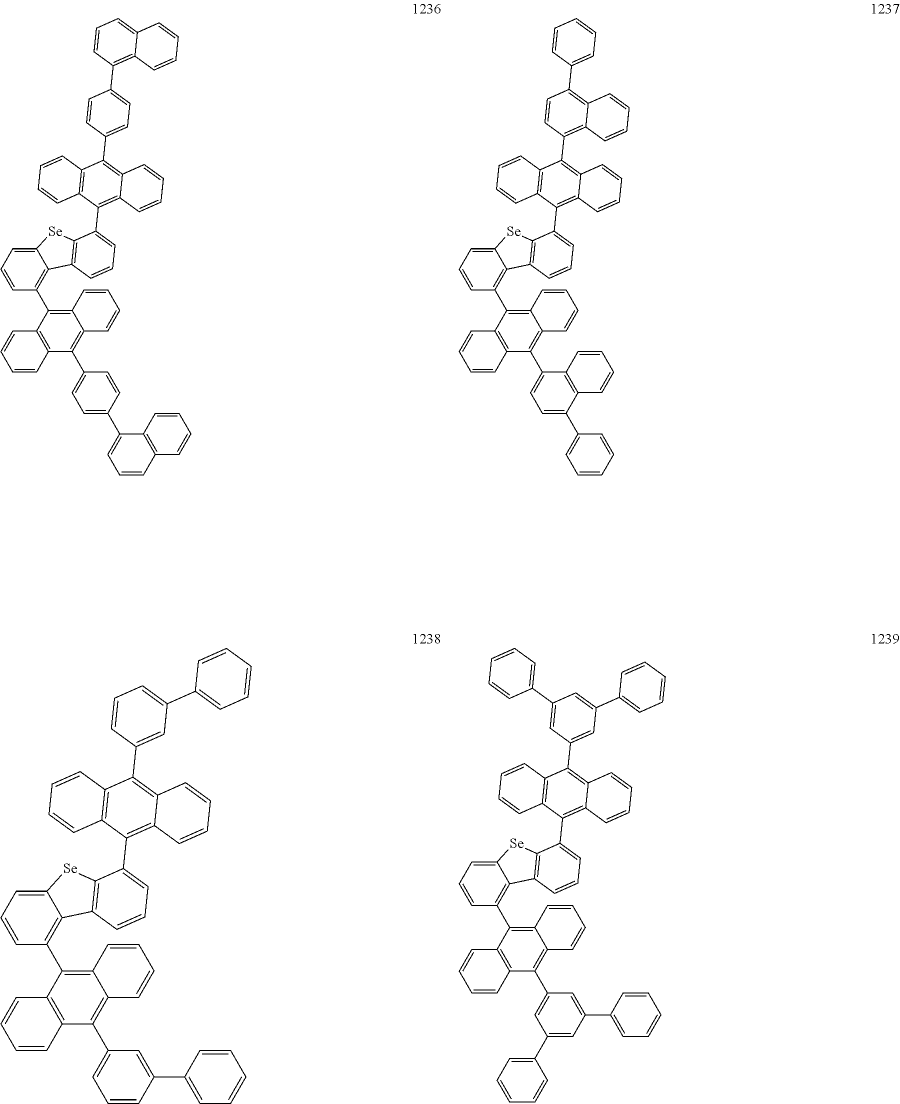

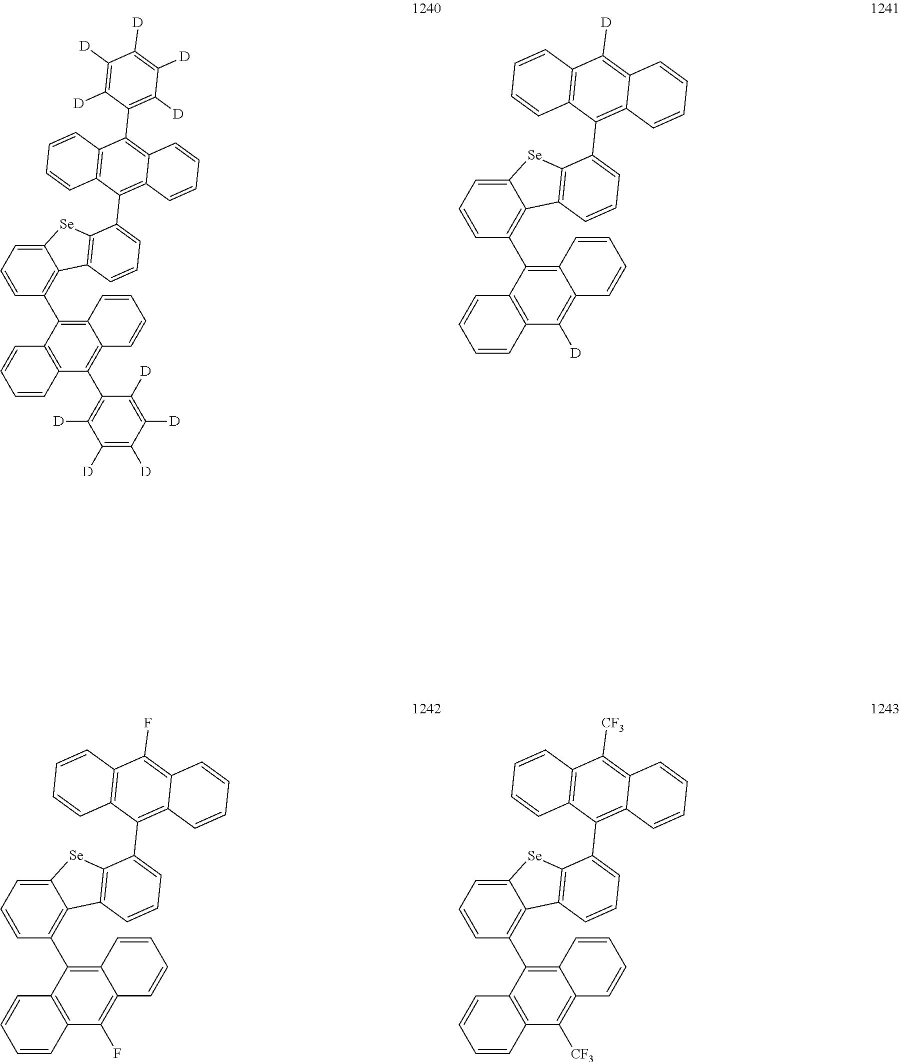 [SMILES: [2H]c1c([2H])c([2H])c(-c2c3ccccc3c(-c3cccc4c3[se]c3cccc(-c5c6ccccc6c(-c6c([2H])c([2H])c([2H])c([2H])c6[2H])c6ccccc56)c34)c3ccccc23)c([2H])c1[2H] ; [2H]c1c2ccccc2c(-c2cccc3c2[se]c2cccc(-c4c5ccccc5c([2H])c5ccccc45)c23)c2ccccc12 ; Fc1c2ccccc2c(-c2cccc3c2[se]c2cccc(-c4c5ccccc5c(F)c5ccccc45)c23)c2ccccc12 ; FC(F)(F)c1c2ccccc2c(-c2cccc3c2[se]c2cccc(-c4c5ccccc5c(C(F)(F)F)c5ccccc45)c23)c2ccccc12]

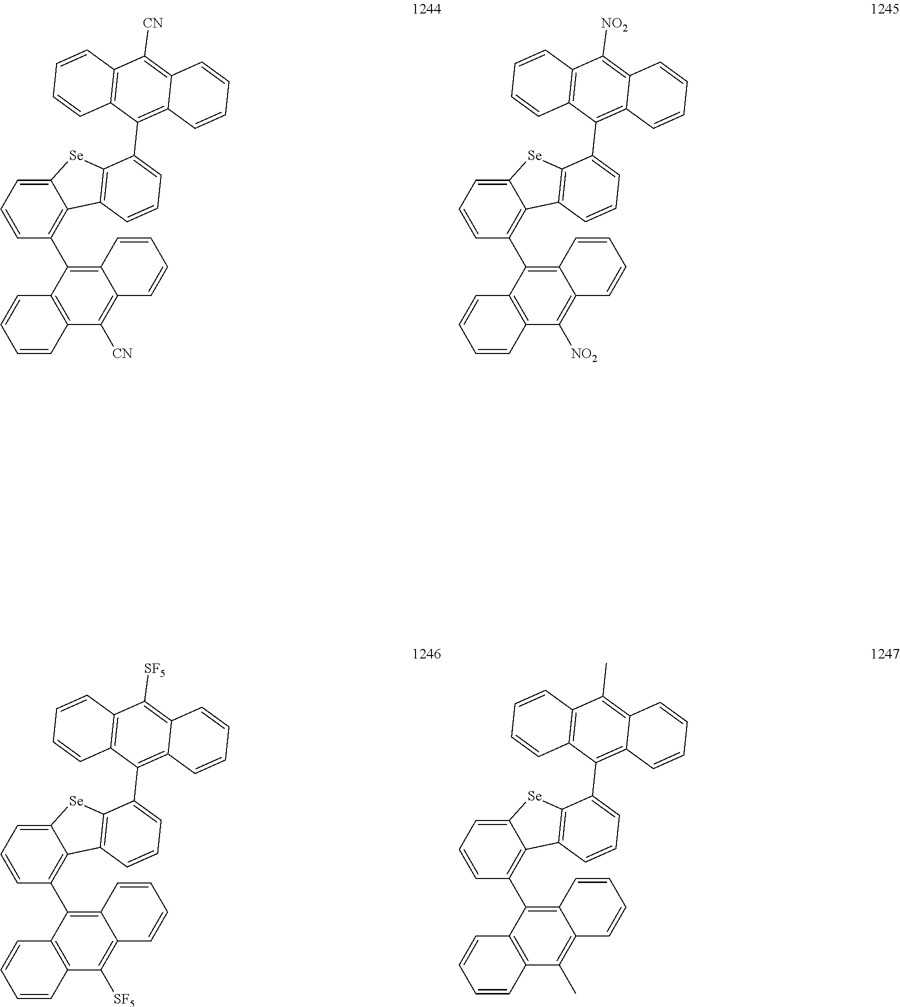

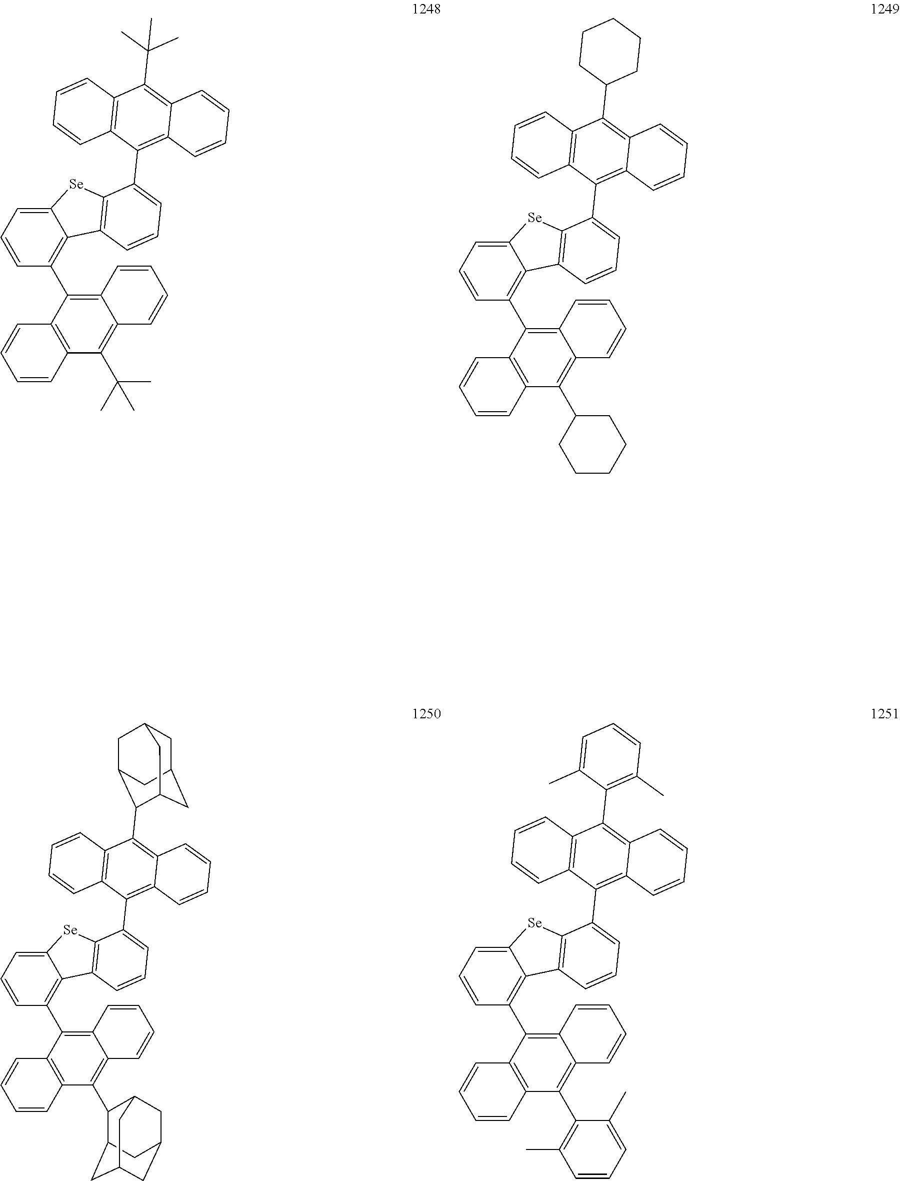 [SMILES: CC(C)(C)c1c2ccccc2c(-c2cccc3c2[se]c2cccc(-c4c5ccccc5c(C(C)(C)C)c5ccccc45)c23)c2ccccc12 ; c1cc(-c2c3ccccc3c(C3CCCCC3)c3ccccc23)c2c(c1)[se]c1c(-c3c4ccccc4c(C4CCCCC4)c4ccccc34)cccc12 ; c1cc(-c2c3ccccc3c(C3C4CC5CC(C4)CC3C5)c3ccccc23)c2c(c1)[se]c1c(-c3c4ccccc4c(C4C5CC6CC(C5)CC4C6)c4ccccc34)cccc12 ; Cc1cccc(C)c1-c1c2ccccc2c(-c2cccc3c2[se]c2cccc(-c4c5ccccc5c(-c5c(C)cccc5C)c5ccccc45)c23)c2ccccc12]

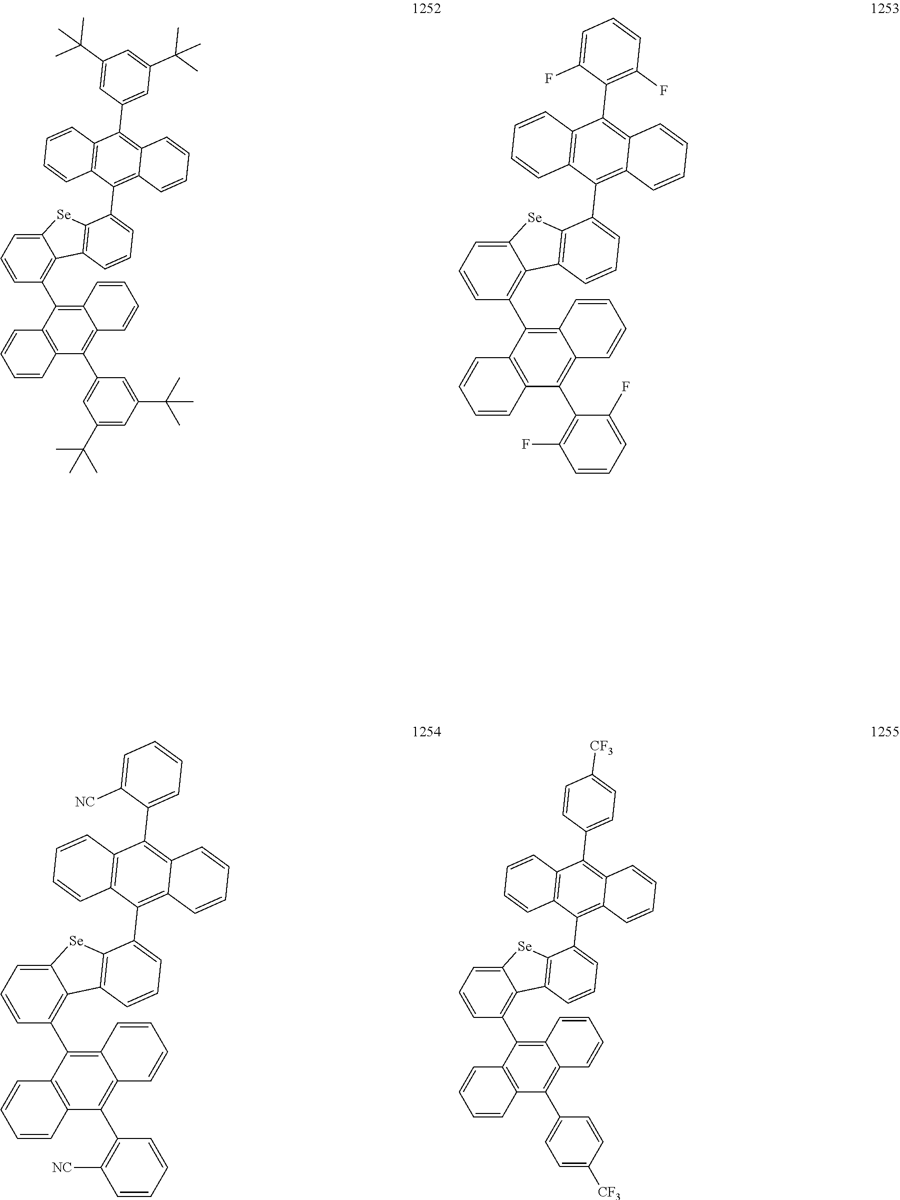 [SMILES: CC(C)(C)c1cc(-c2c3ccccc3c(-c3cccc4c3[se]c3cccc(-c5c6ccccc6c(-c6cc(C(C)(C)C)cc(C(C)(C)C)c6)c6ccccc56)c34)c3ccccc23)cc(C(C)(C)C)c1 ; Fc1cccc(F)c1-c1c2ccccc2c(-c2cccc3c2[se]c2cccc(-c4c5ccccc5c(-c5c(F)cccc5F)c5ccccc45)c23)c2ccccc12 ; N#Cc1ccccc1-c1c2ccccc2c(-c2cccc3c2[se]c2cccc(-c4c5ccccc5c(-c5ccccc5C#N)c5ccccc45)c23)c2ccccc12 ; FC(F)(F)c1ccc(-c2c3ccccc3c(-c3cccc4c3[se]c3cccc(-c5c6ccccc6c(-c6ccc(C(F)(F)F)cc6)c6ccccc56)c34)c3ccccc23)cc1]

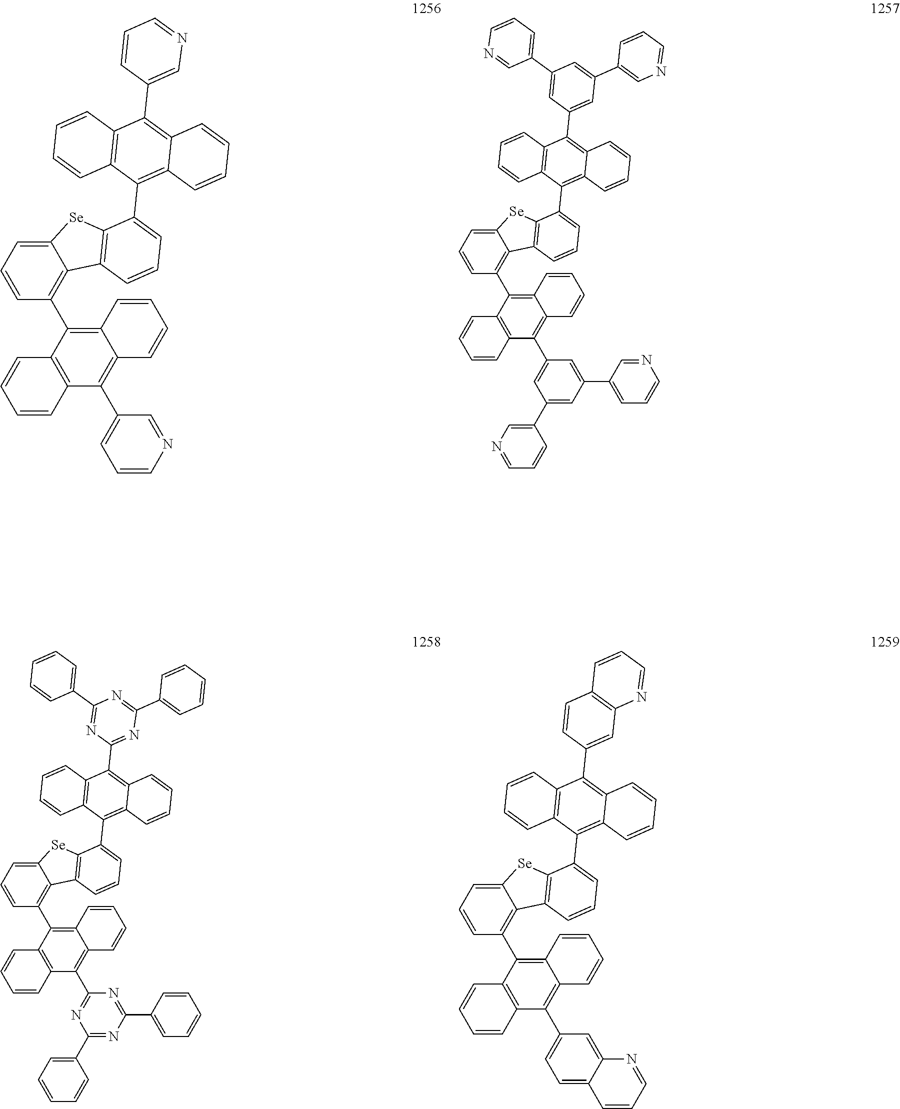 [SMILES: c1cncc(-c2c3ccccc3c(-c3cccc4c3[se]c3cccc(-c5c6ccccc6c(-c6cccnc6)c6ccccc56)c34)c3ccccc23)c1 ; c1cncc(-c2cc(-c3cccnc3)cc(-c3c4ccccc4c(-c4cccc5c4[se]c4cccc(-c6c7ccccc7c(-c7cc(-c8cccnc8)cc(-c8cccnc8)c7)c7ccccc67)c45)c4ccccc34)c2)c1 ; c1ccc(-c2nc(-c3ccccc3)nc(-c3c4ccccc4c(-c4cccc5c4[se]c4cccc(-c6c7ccccc7c(-c7nc(-c8ccccc8)nc(-c8ccccc8)n7)c7ccccc67)c45)c4ccccc34)n2)cc1 ; c1cnc2cc(-c3c4ccccc4c(-c4cccc5c4[se]c4cccc(-c6c7ccccc7c(-c7ccc8cccnc8c7)c7ccccc67)c45)c4ccccc34)ccc2c1]

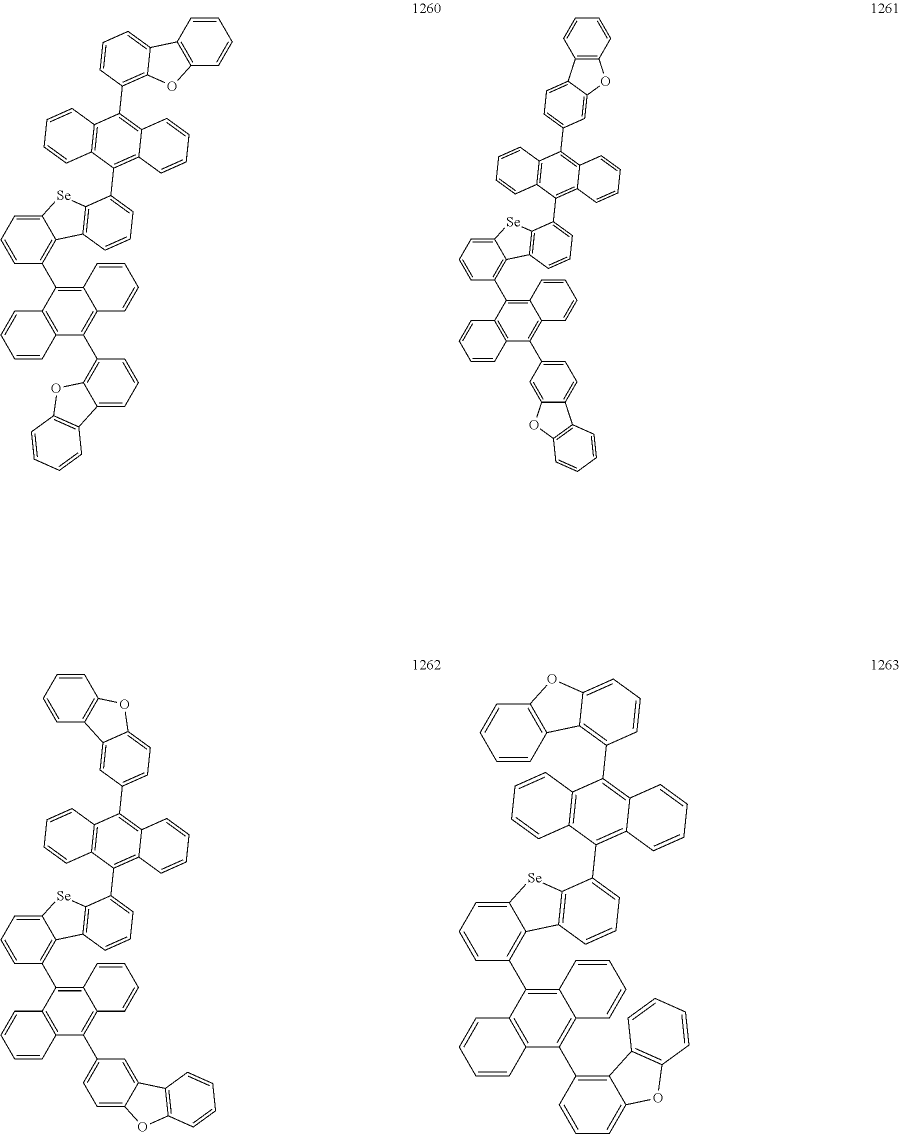 [SMILES: c1ccc2c(c1)oc1c(-c3c4ccccc4c(-c4cccc5c4[se]c4cccc(-c6c7ccccc7c(-c7cccc8c7oc7ccccc78)c7ccccc67)c45)c4ccccc34)cccc12 ; c1ccc2c(c1)oc1cc(-c3c4ccccc4c(-c4cccc5c4[se]c4cccc(-c6c7ccccc7c(-c7ccc8c(c7)oc7ccccc78)c7ccccc67)c45)c4ccccc34)ccc12 ; c1ccc2c(c1)oc1ccc(-c3c4ccccc4c(-c4cccc5c4[se]c4cccc(-c6c7ccccc7c(-c7ccc8oc9ccccc9c8c7)c7ccccc67)c45)c4ccccc34)cc12 ; c1ccc2c(c1)oc1cccc(-c3c4ccccc4c(-c4cccc5c4[se]c4cccc(-c6c7ccccc7c(-c7cccc8oc9ccccc9c78)c7ccccc67)c45)c4ccccc34)c12]

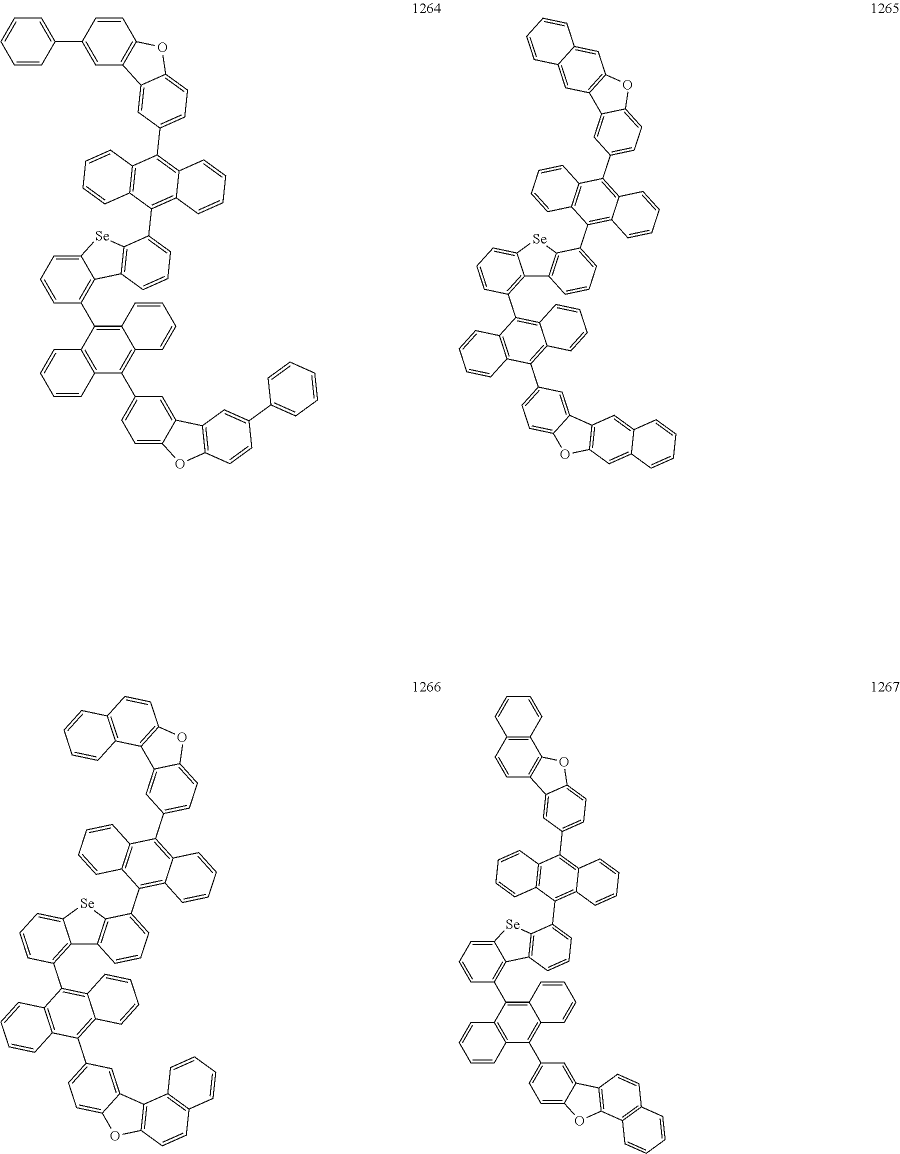

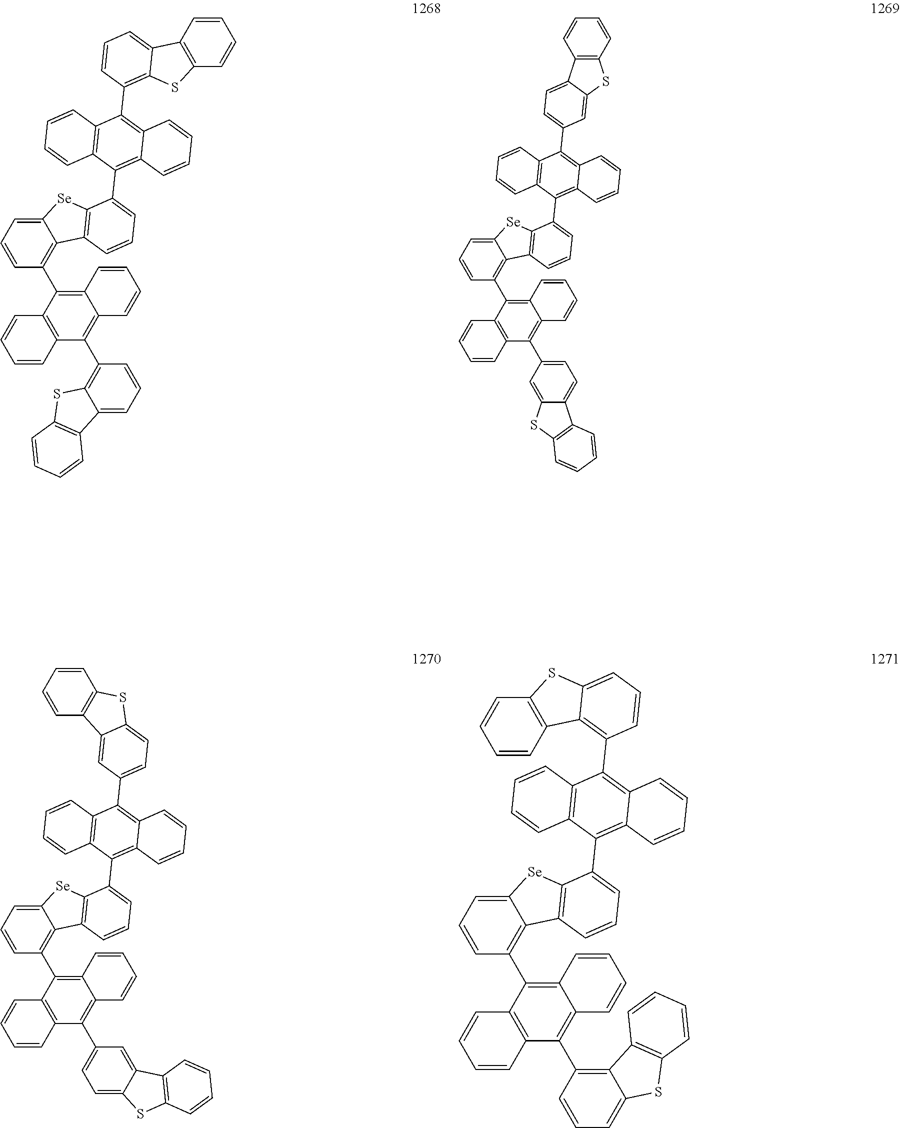 [SMILES: c1ccc2c(c1)sc1c(-c3c4ccccc4c(-c4cccc5c4[se]c4cccc(-c6c7ccccc7c(-c7cccc8c7sc7ccccc78)c7ccccc67)c45)c4ccccc34)cccc12 ; c1ccc2c(c1)sc1cc(-c3c4ccccc4c(-c4cccc5c4[se]c4cccc(-c6c7ccccc7c(-c7ccc8c(c7)sc7ccccc78)c7ccccc67)c45)c4ccccc34)ccc12 ; c1ccc2c(c1)sc1ccc(-c3c4ccccc4c(-c4cccc5c4[se]c4cccc(-c6c7ccccc7c(-c7ccc8sc9ccccc9c8c7)c7ccccc67)c45)c4ccccc34)cc12 ; c1ccc2c(c1)sc1cccc(-c3c4ccccc4c(-c4cccc5c4[se]c4cccc(-c6c7ccccc7c(-c7cccc8sc9ccccc9c78)c7ccccc67)c45)c4ccccc34)c12]

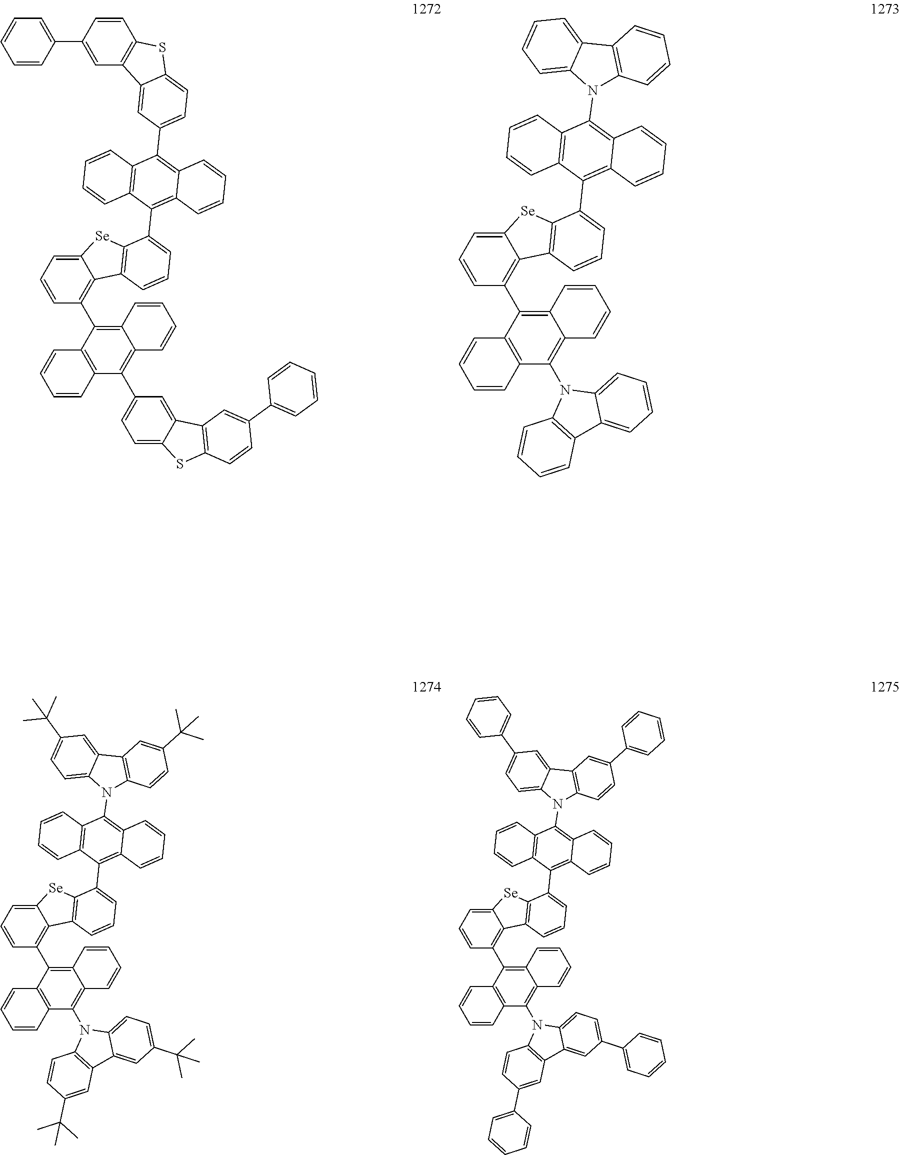

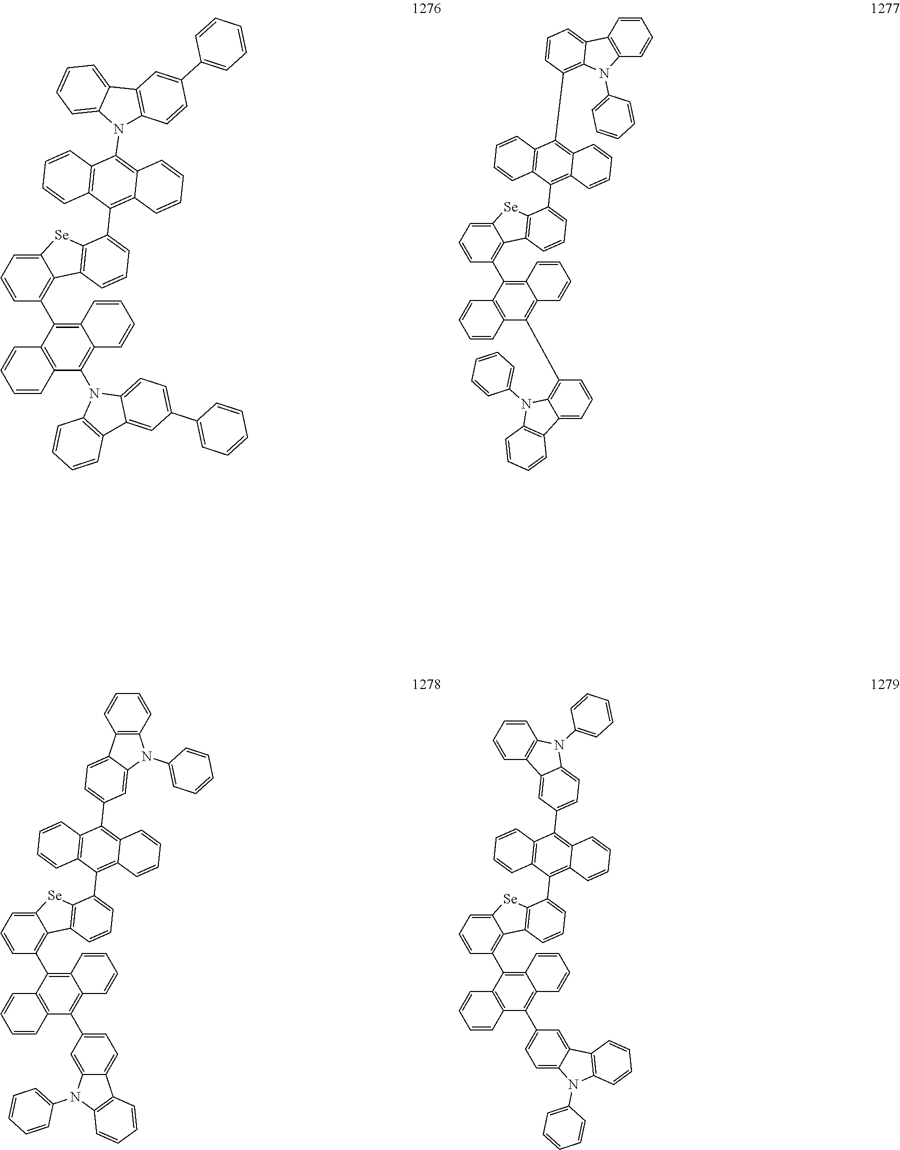

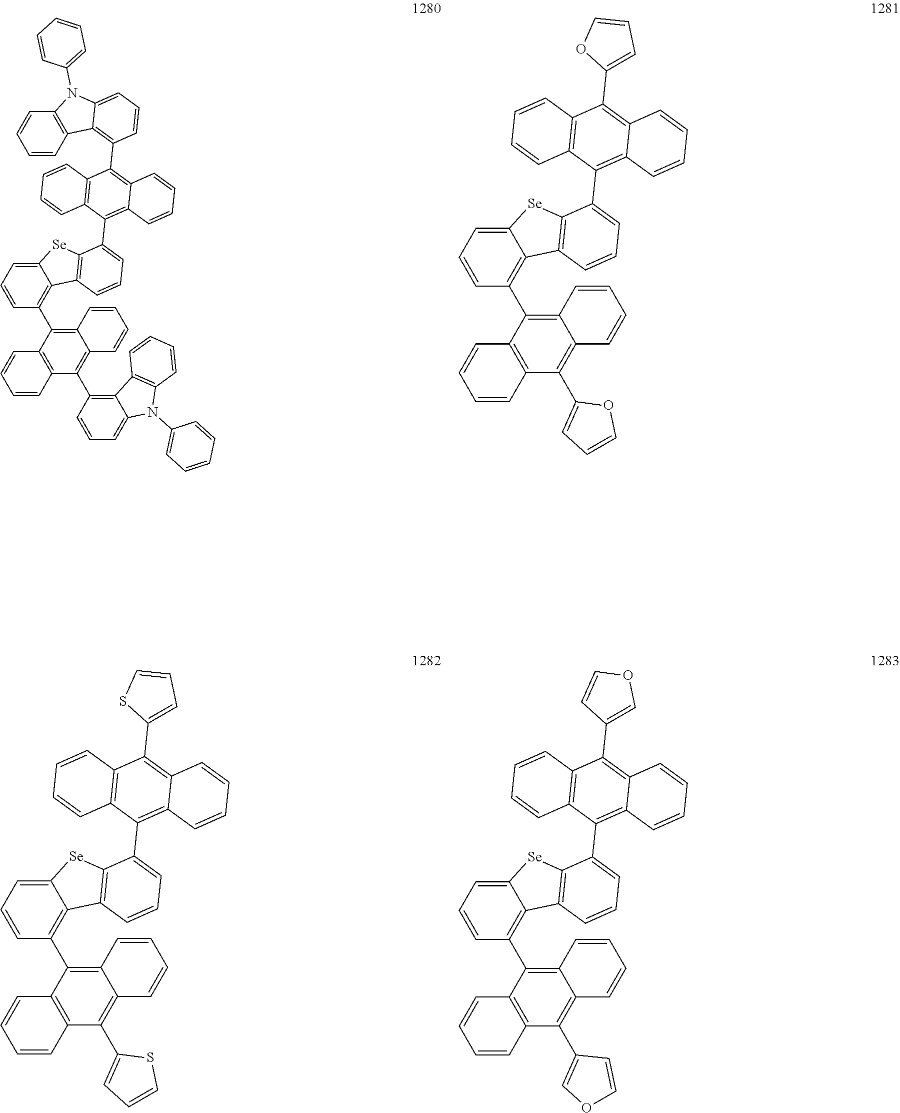 [SMILES: c1ccc(-n2c3ccccc3c3c(-c4c5ccccc5c(-c5cccc6c5[se]c5cccc(-c7c8ccccc8c(-c8cccc9c8c8ccccc8n9-c8ccccc8)c8ccccc78)c56)c5ccccc45)cccc32)cc1 ; c1coc(-c2c3ccccc3c(-c3cccc4c3[se]c3cccc(-c5c6ccccc6c(-c6ccco6)c6ccccc56)c34)c3ccccc23)c1 ; c1csc(-c2c3ccccc3c(-c3cccc4c3[se]c3cccc(-c5c6ccccc6c(-c6cccs6)c6ccccc56)c34)c3ccccc23)c1 ; c1cc(-c2c3ccccc3c(-c3ccoc3)c3ccccc23)c2c(c1)[se]c1c(-c3c4ccccc4c(-c4ccoc4)c4ccccc34)cccc12]

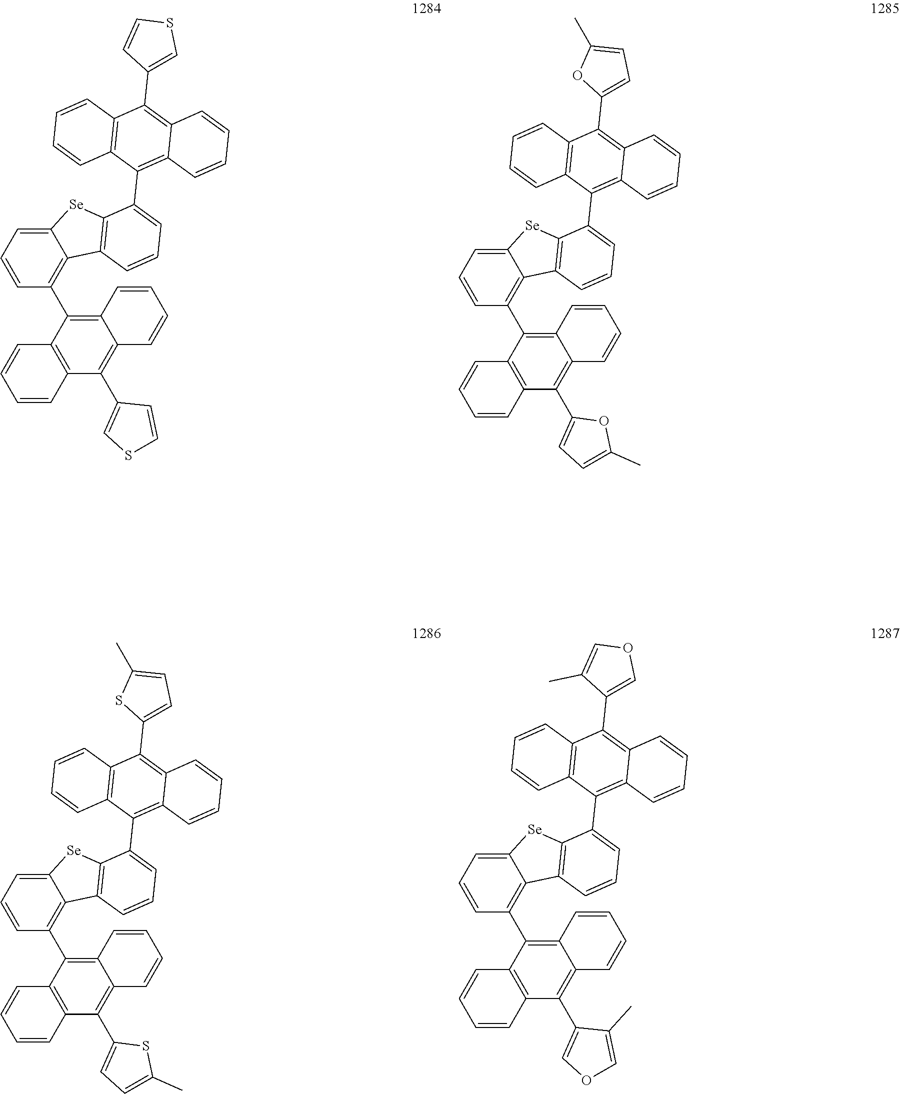 [SMILES: c1cc(-c2c3ccccc3c(-c3ccsc3)c3ccccc23)c2c(c1)[se]c1c(-c3c4ccccc4c(-c4ccsc4)c4ccccc34)cccc12 ; Cc1ccc(-c2c3ccccc3c(-c3cccc4c3[se]c3cccc(-c5c6ccccc6c(-c6ccc(C)o6)c6ccccc56)c34)c3ccccc23)o1 ; Cc1ccc(-c2c3ccccc3c(-c3cccc4c3[se]c3cccc(-c5c6ccccc6c(-c6ccc(C)s6)c6ccccc56)c34)c3ccccc23)s1 ; Cc1cocc1-c1c2ccccc2c(-c2cccc3c2[se]c2cccc(-c4c5ccccc5c(-c5cocc5C)c5ccccc45)c23)c2ccccc12]

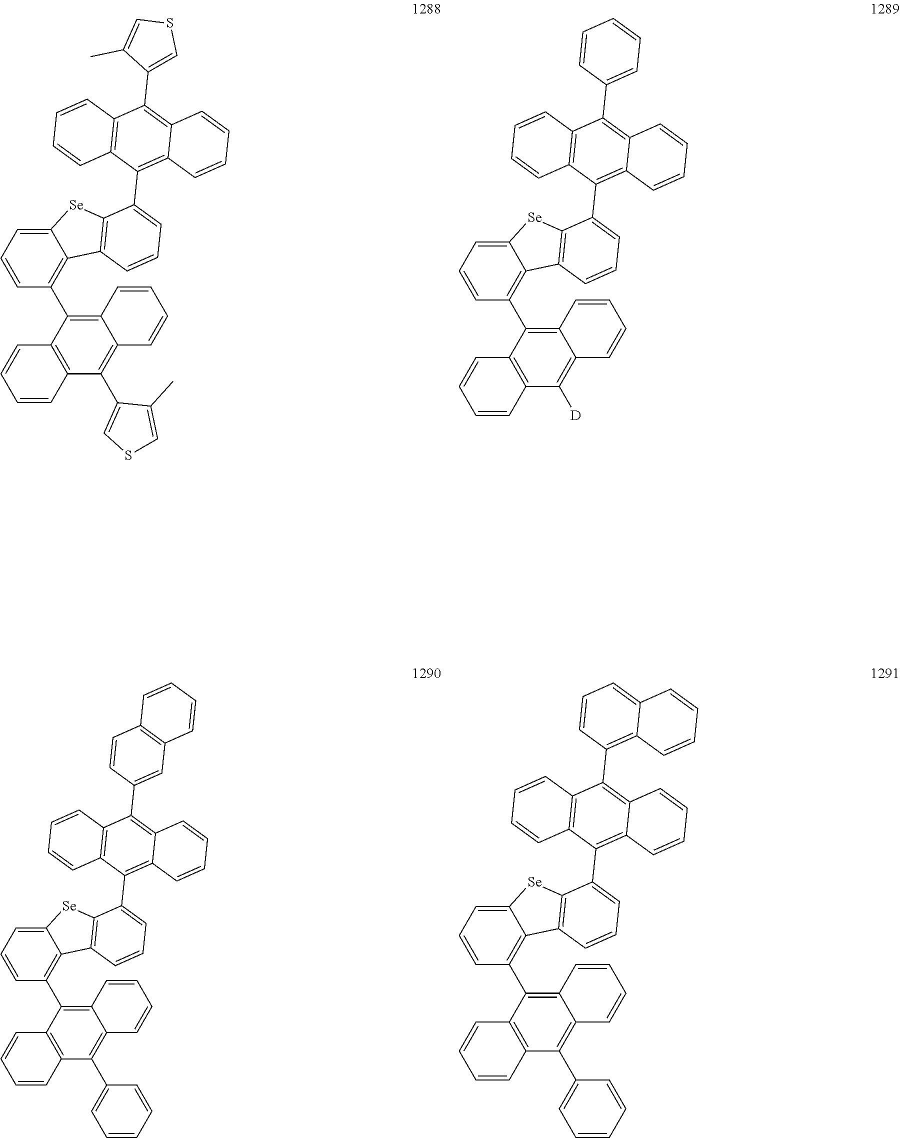 [SMILES: Cc1cscc1-c1c2ccccc2c(-c2cccc3c2[se]c2cccc(-c4c5ccccc5c(-c5cscc5C)c5ccccc45)c23)c2ccccc12 ; [2H]c1c2ccccc2c(-c2cccc3[se]c4c(-c5c6ccccc6c(-c6ccccc6)c6ccccc56)cccc4c23)c2ccccc12 ; c1ccc(-c2c3ccccc3c(-c3cccc4[se]c5c(-c6c7ccccc7c(-c7ccc8ccccc8c7)c7ccccc67)cccc5c34)c3ccccc23)cc1 ; c1ccc(-c2c3ccccc3c(-c3cccc4[se]c5c(-c6c7ccccc7c(-c7cccc8ccccc78)c7ccccc67)cccc5c34)c3ccccc23)cc1]

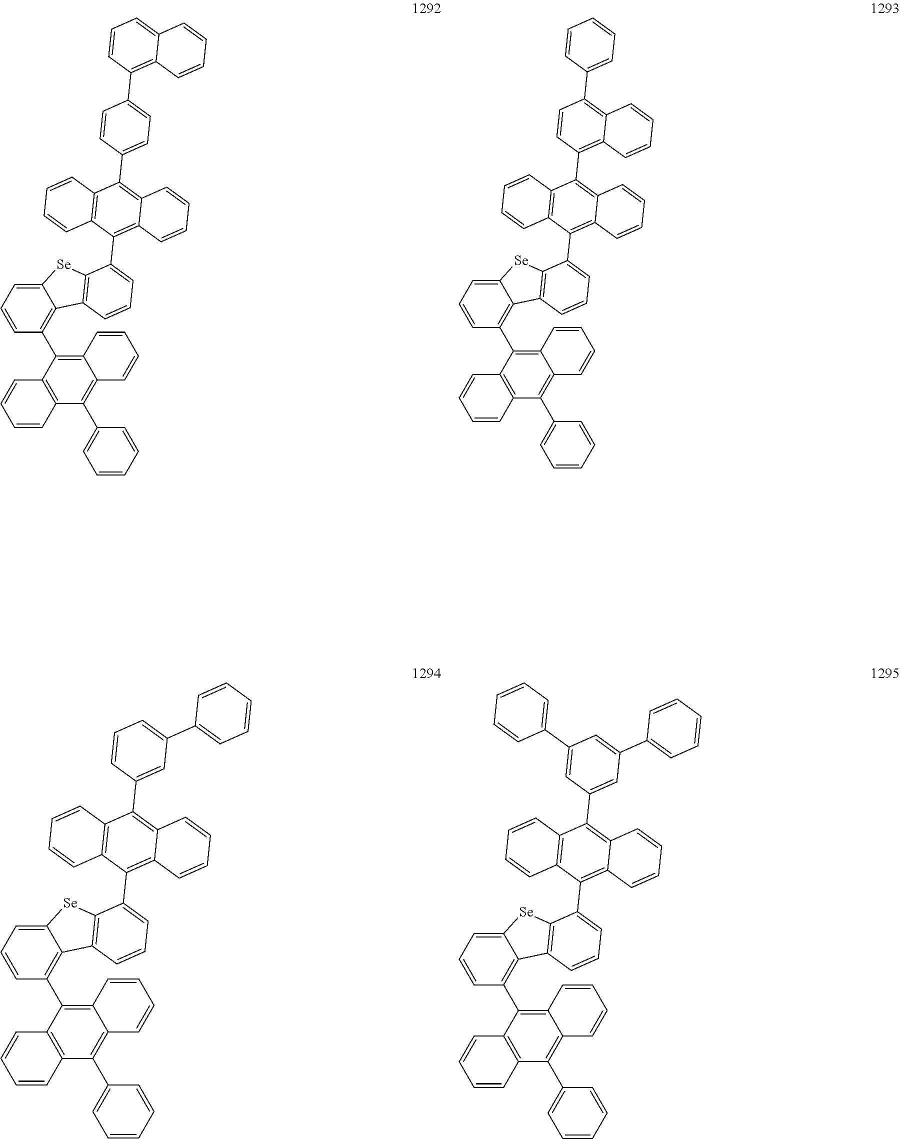 [SMILES: c1ccc(-c2c3ccccc3c(-c3cccc4[se]c5c(-c6c7ccccc7c(-c7ccc(-c8cccc9ccccc89)cc7)c7ccccc67)cccc5c34)c3ccccc23)cc1 ; c1ccc(-c2ccc(-c3c4ccccc4c(-c4cccc5c4[se]c4cccc(-c6c7ccccc7c(-c7ccccc7)c7ccccc67)c45)c4ccccc34)c3ccccc23)cc1 ; c1ccc(-c2cccc(-c3c4ccccc4c(-c4cccc5c4[se]c4cccc(-c6c7ccccc7c(-c7ccccc7)c7ccccc67)c45)c4ccccc34)c2)cc1 ; c1ccc(-c2cc(-c3ccccc3)cc(-c3c4ccccc4c(-c4cccc5c4[se]c4cccc(-c6c7ccccc7c(-c7ccccc7)c7ccccc67)c45)c4ccccc34)c2)cc1]

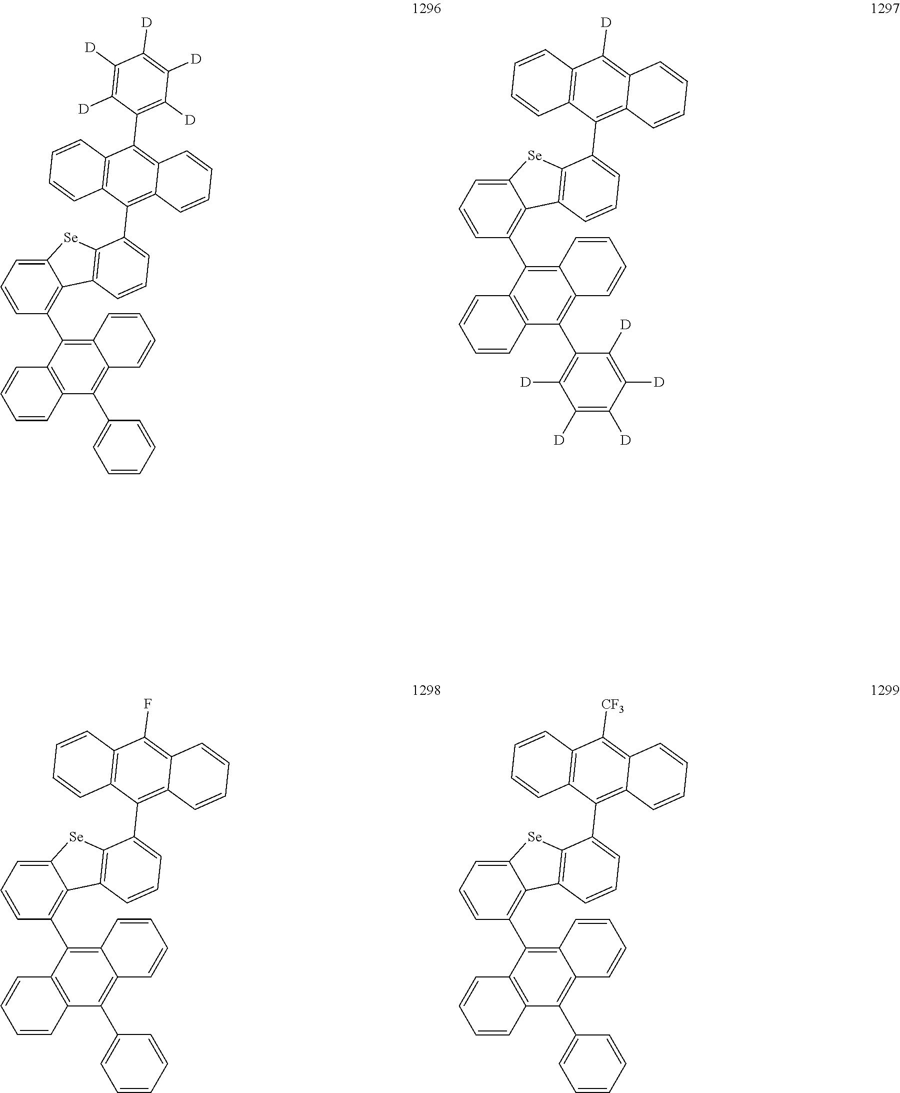 [SMILES: [2H]c1c([2H])c([2H])c(-c2c3ccccc3c(-c3cccc4c3[se]c3cccc(-c5c6ccccc6c(-c6ccccc6)c6ccccc56)c34)c3ccccc23)c([2H])c1[2H] ; [2H]c1c([2H])c([2H])c(-c2c3ccccc3c(-c3cccc4[se]c5c(-c6c7ccccc7c([2H])c7ccccc67)cccc5c34)c3ccccc23)c([2H])c1[2H] ; Fc1c2ccccc2c(-c2cccc3c2[se]c2cccc(-c4c5ccccc5c(-c5ccccc5)c5ccccc45)c23)c2ccccc12 ; FC(F)(F)c1c2ccccc2c(-c2cccc3c2[se]c2cccc(-c4c5ccccc5c(-c5ccccc5)c5ccccc45)c23)c2ccccc12]

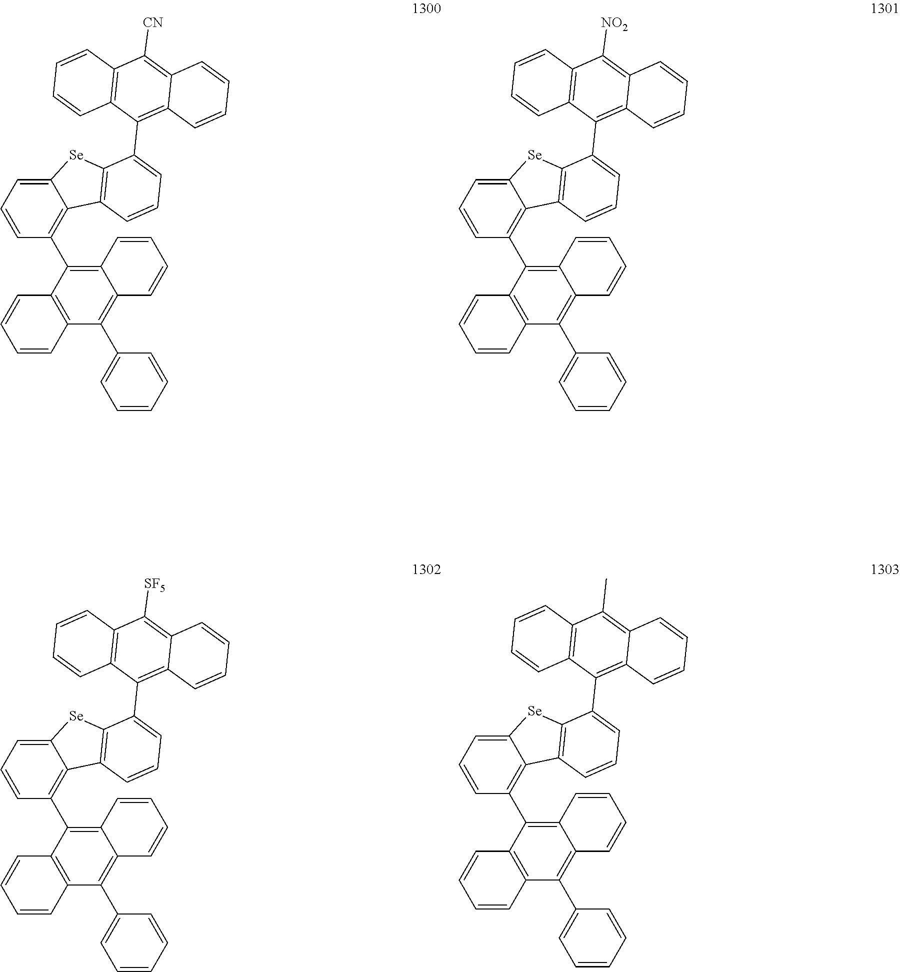

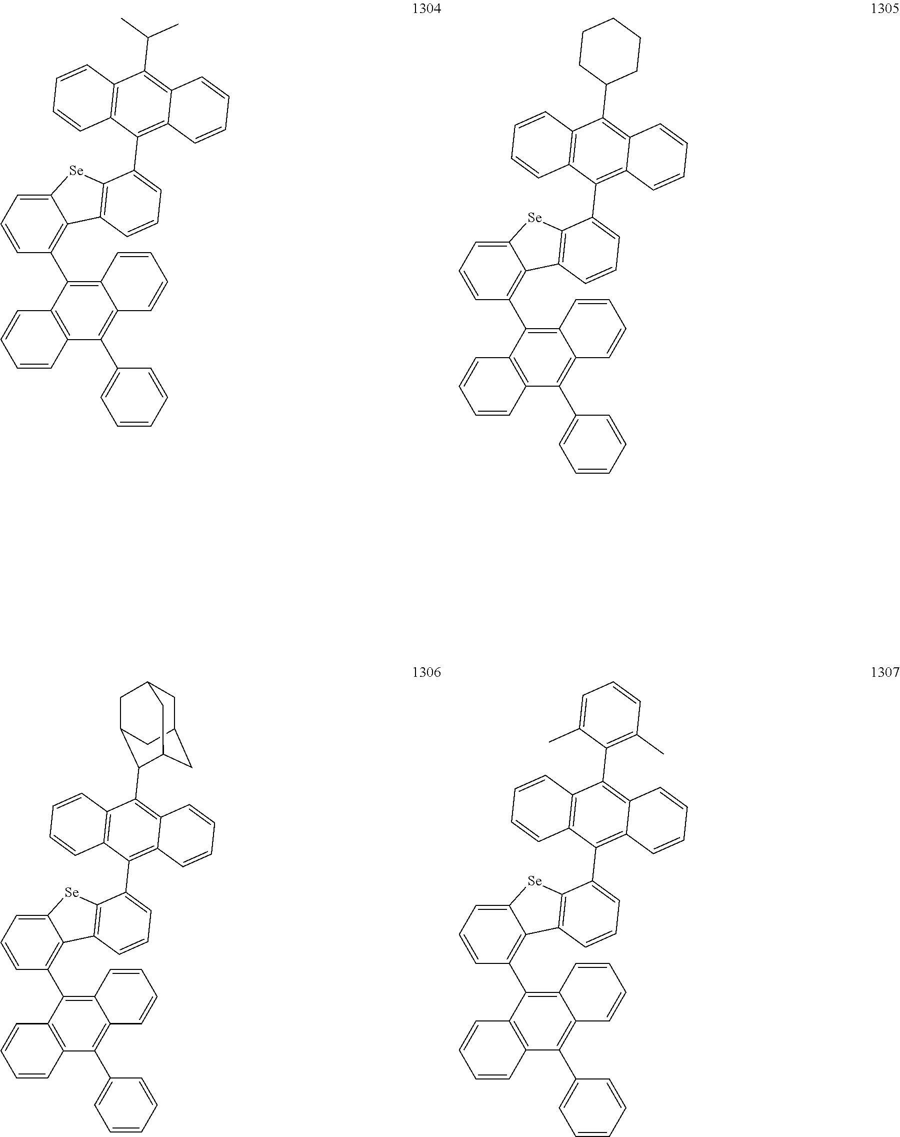 [SMILES: CC(C)c1c2ccccc2c(-c2cccc3c2[se]c2cccc(-c4c5ccccc5c(-c5ccccc5)c5ccccc45)c23)c2ccccc12 ; c1ccc(-c2c3ccccc3c(-c3cccc4[se]c5c(-c6c7ccccc7c(C7CCCCC7)c7ccccc67)cccc5c34)c3ccccc23)cc1 ; c1ccc(-c2c3ccccc3c(-c3cccc4[se]c5c(-c6c7ccccc7c(C7C8CC9CC(C8)CC7C9)c7ccccc67)cccc5c34)c3ccccc23)cc1 ; Cc1cccc(C)c1-c1c2ccccc2c(-c2cccc3c2[se]c2cccc(-c4c5ccccc5c(-c5ccccc5)c5ccccc45)c23)c2ccccc12]

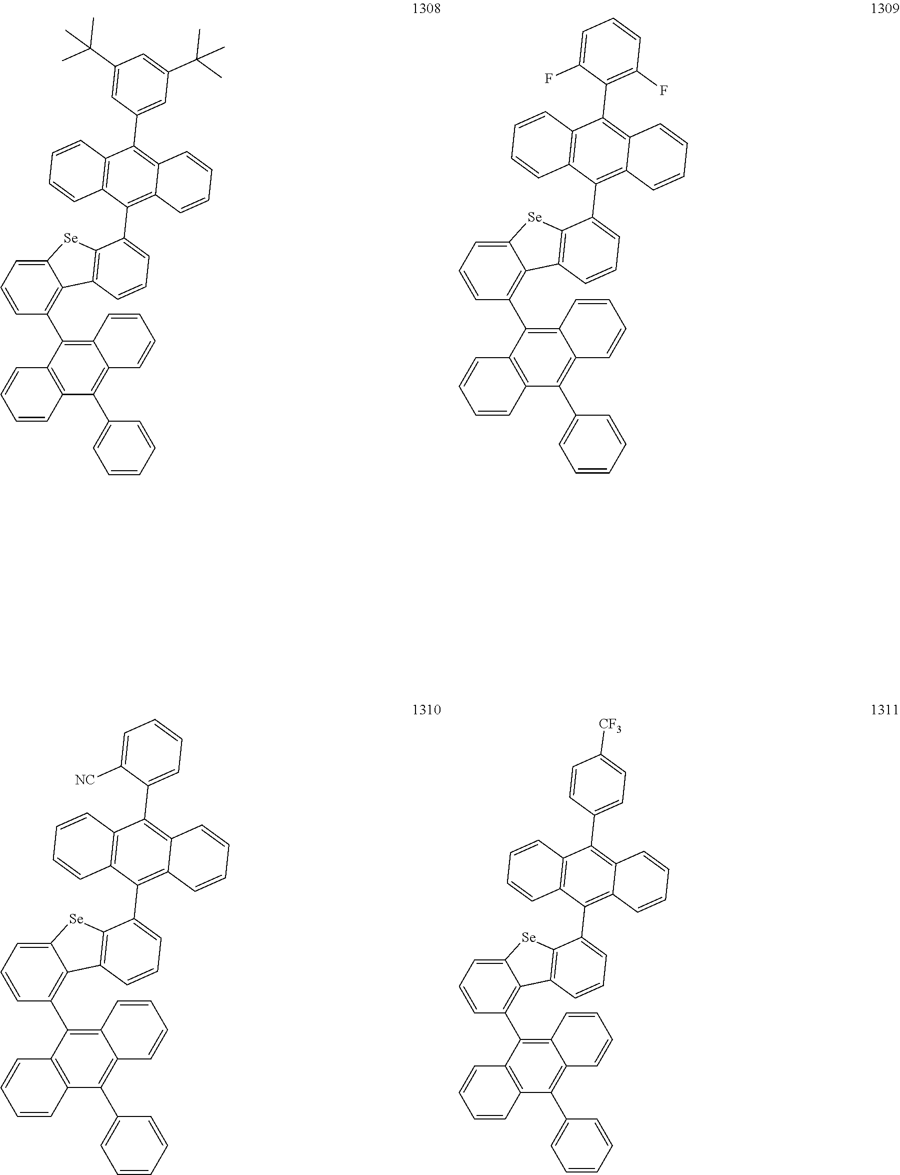 [SMILES: CC(C)(C)c1cc(-c2c3ccccc3c(-c3cccc4c3[se]c3cccc(-c5c6ccccc6c(-c6ccccc6)c6ccccc56)c34)c3ccccc23)cc(C(C)(C)C)c1 ; Fc1cccc(F)c1-c1c2ccccc2c(-c2cccc3c2[se]c2cccc(-c4c5ccccc5c(-c5ccccc5)c5ccccc45)c23)c2ccccc12 ; N#Cc1ccccc1-c1c2ccccc2c(-c2cccc3c2[se]c2cccc(-c4c5ccccc5c(-c5ccccc5)c5ccccc45)c23)c2ccccc12 ; FC(F)(F)c1ccc(-c2c3ccccc3c(-c3cccc4c3[se]c3cccc(-c5c6ccccc6c(-c6ccccc6)c6ccccc56)c34)c3ccccc23)cc1]

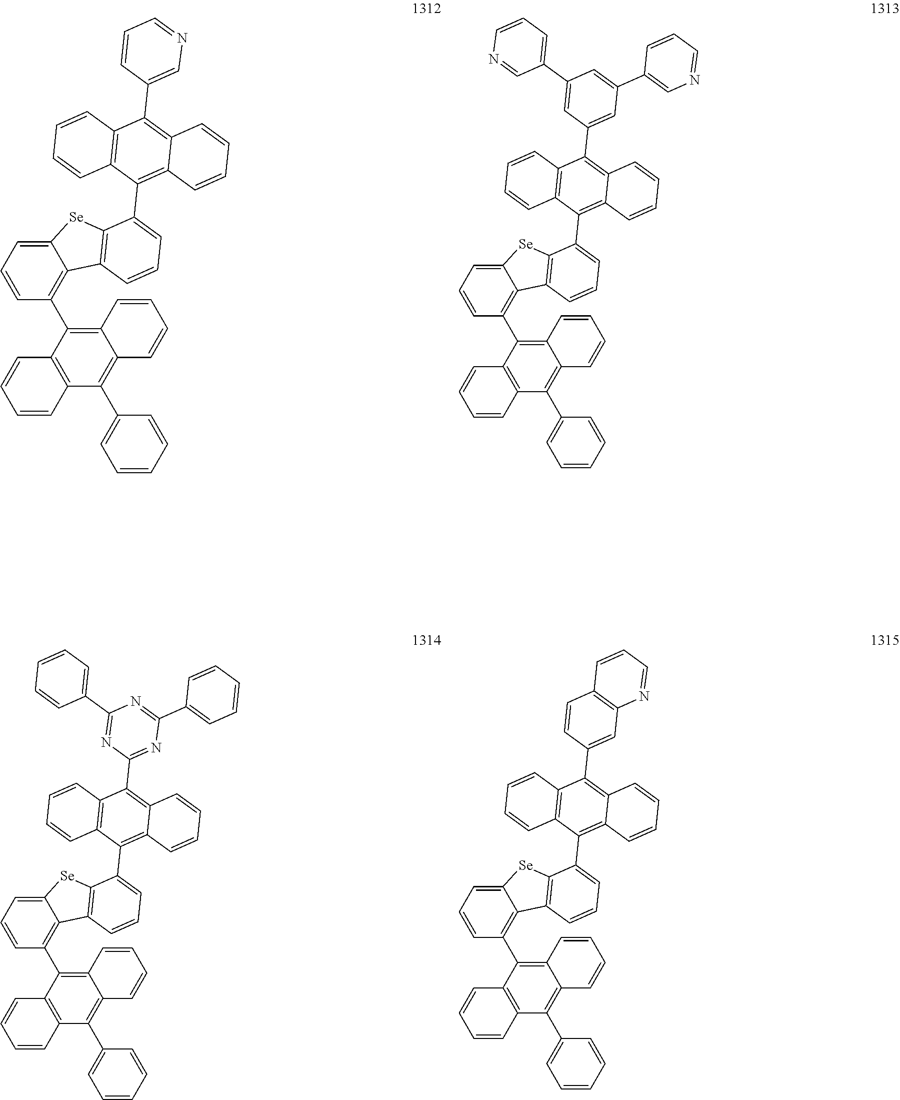 [SMILES: c1ccc(-c2c3ccccc3c(-c3cccc4[se]c5c(-c6c7ccccc7c(-c7cccnc7)c7ccccc67)cccc5c34)c3ccccc23)cc1 ; c1ccc(-c2c3ccccc3c(-c3cccc4[se]c5c(-c6c7ccccc7c(-c7cc(-c8cccnc8)cc(-c8cccnc8)c7)c7ccccc67)cccc5c34)c3ccccc23)cc1 ; c1ccc(-c2nc(-c3ccccc3)nc(-c3c4ccccc4c(-c4cccc5c4[se]c4cccc(-c6c7ccccc7c(-c7ccccc7)c7ccccc67)c45)c4ccccc34)n2)cc1 ; c1ccc(-c2c3ccccc3c(-c3cccc4[se]c5c(-c6c7ccccc7c(-c7ccc8cccnc8c7)c7ccccc67)cccc5c34)c3ccccc23)cc1]

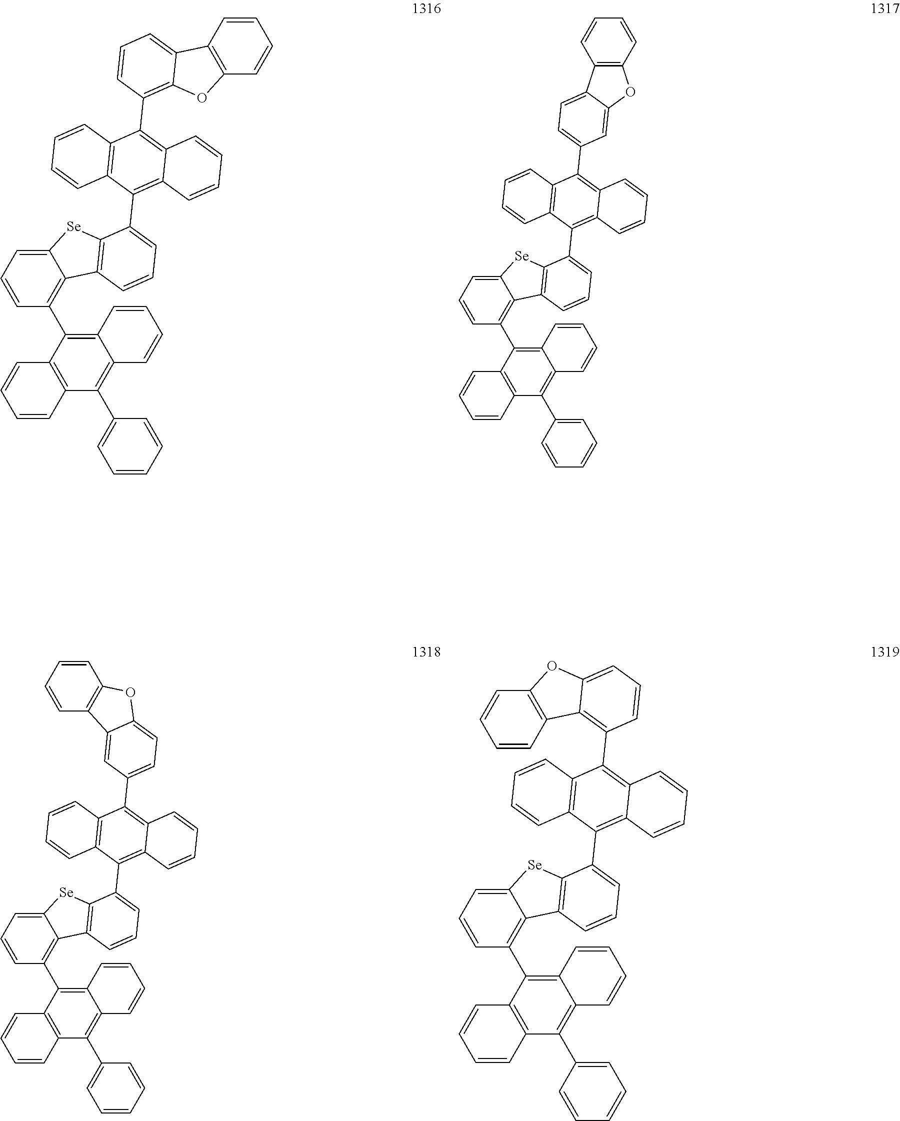 [SMILES: c1ccc(-c2c3ccccc3c(-c3cccc4[se]c5c(-c6c7ccccc7c(-c7cccc8c7oc7ccccc78)c7ccccc67)cccc5c34)c3ccccc23)cc1 ; c1ccc(-c2c3ccccc3c(-c3cccc4[se]c5c(-c6c7ccccc7c(-c7ccc8c(c7)oc7ccccc78)c7ccccc67)cccc5c34)c3ccccc23)cc1 ; c1ccc(-c2c3ccccc3c(-c3cccc4[se]c5c(-c6c7ccccc7c(-c7ccc8oc9ccccc9c8c7)c7ccccc67)cccc5c34)c3ccccc23)cc1 ; c1ccc(-c2c3ccccc3c(-c3cccc4[se]c5c(-c6c7ccccc7c(-c7cccc8oc9ccccc9c78)c7ccccc67)cccc5c34)c3ccccc23)cc1]

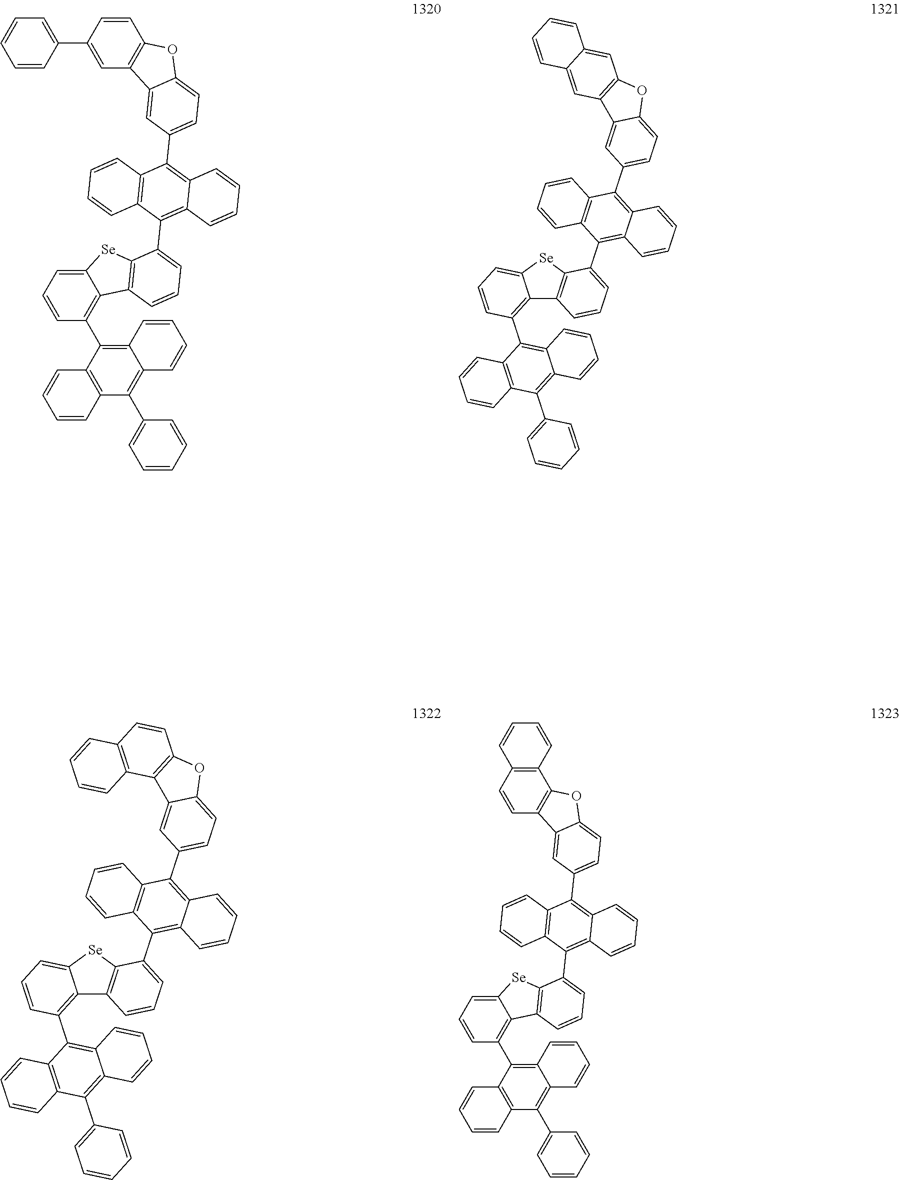 [SMILES: c1ccc(-c2ccc3oc4ccc(-c5c6ccccc6c(-c6cccc7c6[se]c6cccc(-c8c9ccccc9c(-c9ccccc9)c9ccccc89)c67)c6ccccc56)cc4c3c2)cc1 ; c1ccc(-c2c3ccccc3c(-c3cccc4[se]c5c(-c6c7ccccc7c(-c7ccc8oc9cc%10ccccc%10cc9c8c7)c7ccccc67)cccc5c34)c3ccccc23)cc1 ; c1ccc(-c2c3ccccc3c(-c3cccc4[se]c5c(-c6c7ccccc7c(-c7ccc8oc9ccc%10ccccc%10c9c8c7)c7ccccc67)cccc5c34)c3ccccc23)cc1 ; c1ccc(-c2c3ccccc3c(-c3cccc4[se]c5c(-c6c7ccccc7c(-c7ccc8oc9c%10ccccc%10ccc9c8c7)c7ccccc67)cccc5c34)c3ccccc23)cc1]

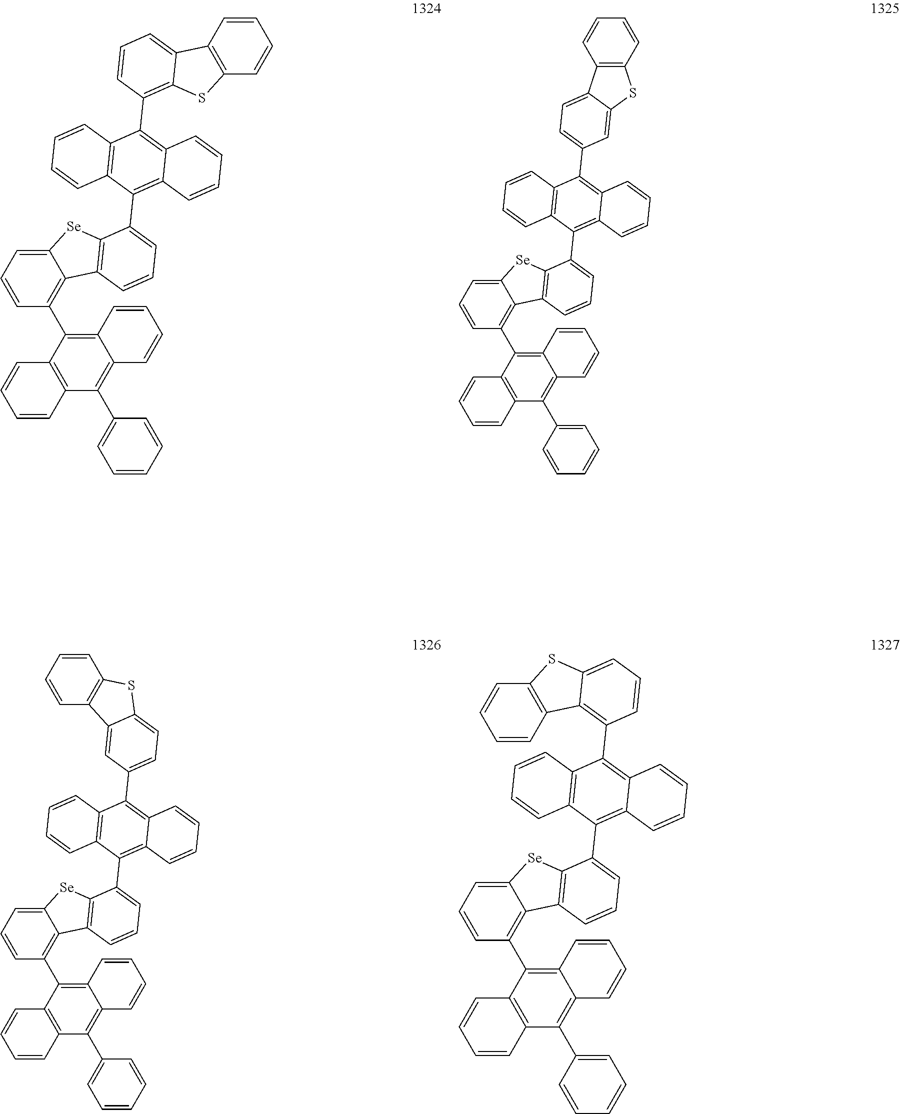 [SMILES: c1ccc(-c2c3ccccc3c(-c3cccc4[se]c5c(-c6c7ccccc7c(-c7cccc8c7sc7ccccc78)c7ccccc67)cccc5c34)c3ccccc23)cc1 ; c1ccc(-c2c3ccccc3c(-c3cccc4[se]c5c(-c6c7ccccc7c(-c7ccc8c(c7)sc7ccccc78)c7ccccc67)cccc5c34)c3ccccc23)cc1 ; c1ccc(-c2c3ccccc3c(-c3cccc4[se]c5c(-c6c7ccccc7c(-c7ccc8sc9ccccc9c8c7)c7ccccc67)cccc5c34)c3ccccc23)cc1 ; c1ccc(-c2c3ccccc3c(-c3cccc4[se]c5c(-c6c7ccccc7c(-c7cccc8sc9ccccc9c78)c7ccccc67)cccc5c34)c3ccccc23)cc1]

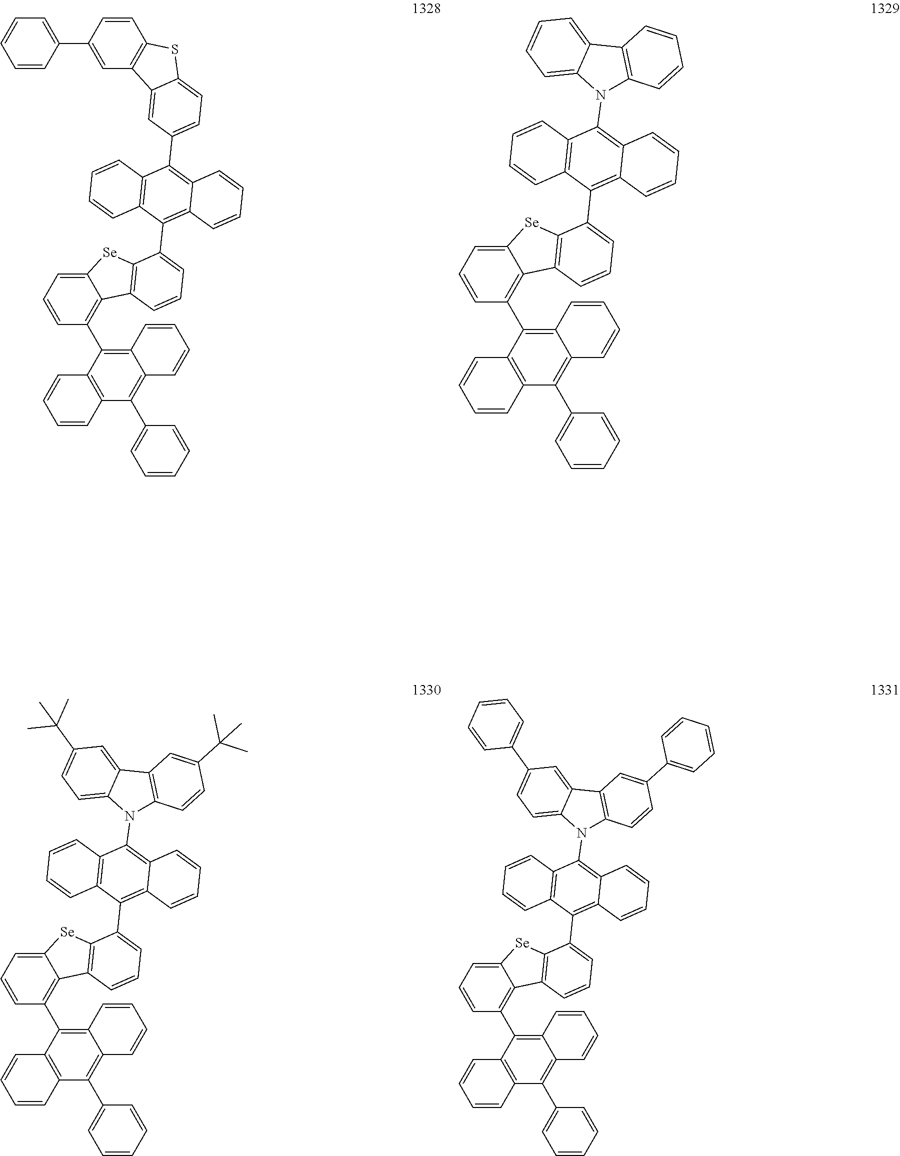 [SMILES: c1ccc(-c2ccc3sc4ccc(-c5c6ccccc6c(-c6cccc7c6[se]c6cccc(-c8c9ccccc9c(-c9ccccc9)c9ccccc89)c67)c6ccccc56)cc4c3c2)cc1 ; c1ccc(-c2c3ccccc3c(-c3cccc4[se]c5c(-c6c7ccccc7c(-n7c8ccccc8c8ccccc87)c7ccccc67)cccc5c34)c3ccccc23)cc1 ; CC(C)(C)c1ccc2c(c1)c1cc(C(C)(C)C)ccc1n2-c1c2ccccc2c(-c2cccc3c2[se]c2cccc(-c4c5ccccc5c(-c5ccccc5)c5ccccc45)c23)c2ccccc12 ; c1ccc(-c2ccc3c(c2)c2cc(-c4ccccc4)ccc2n3-c2c3ccccc3c(-c3cccc4c3[se]c3cccc(-c5c6ccccc6c(-c6ccccc6)c6ccccc56)c34)c3ccccc23)cc1]

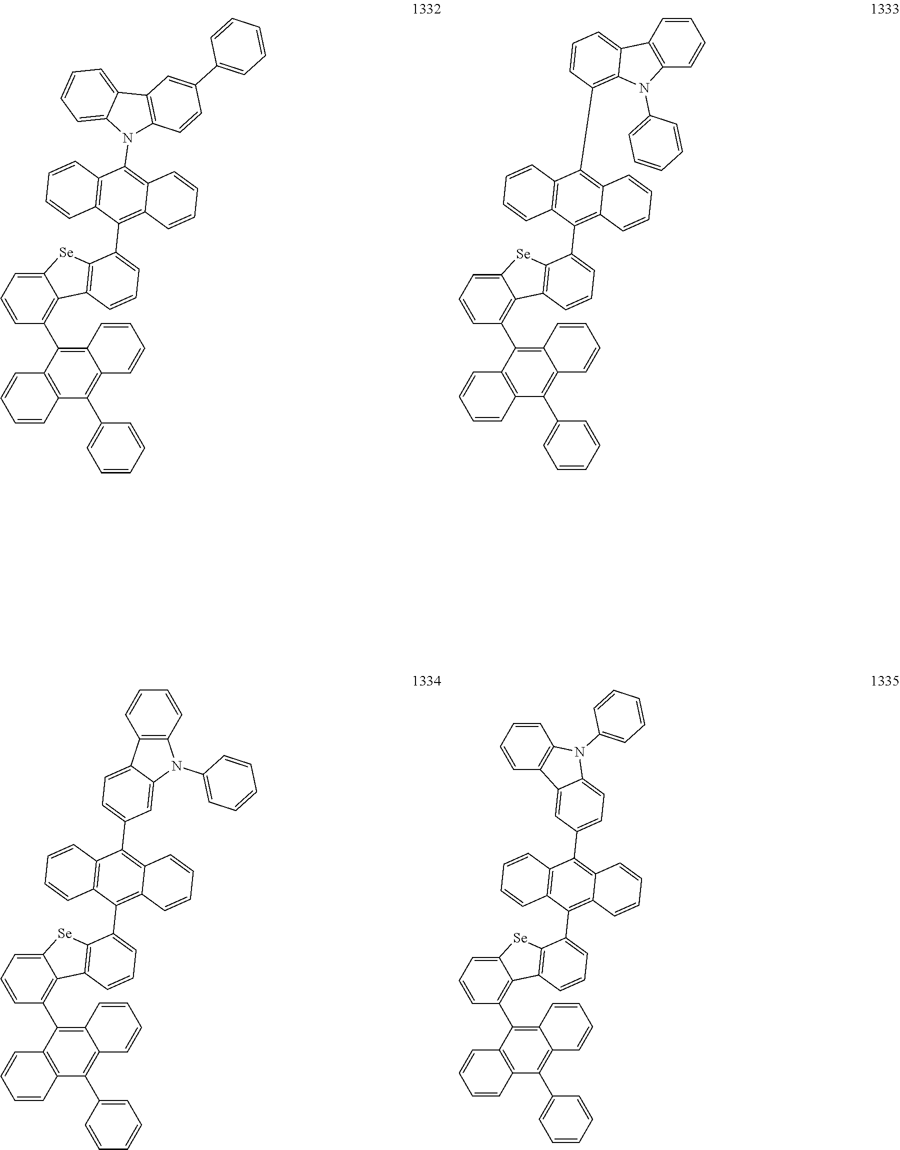 [SMILES: c1ccc(-c2ccc3c(c2)c2ccccc2n3-c2c3ccccc3c(-c3cccc4c3[se]c3cccc(-c5c6ccccc6c(-c6ccccc6)c6ccccc56)c34)c3ccccc23)cc1 ; c1ccc(-c2c3ccccc3c(-c3cccc4[se]c5c(-c6c7ccccc7c(-c7cccc8c9ccccc9n(-c9ccccc9)c78)c7ccccc67)cccc5c34)c3ccccc23)cc1 ; c1ccc(-c2c3ccccc3c(-c3cccc4[se]c5c(-c6c7ccccc7c(-c7ccc8c9ccccc9n(-c9ccccc9)c8c7)c7ccccc67)cccc5c34)c3ccccc23)cc1 ; c1ccc(-c2c3ccccc3c(-c3cccc4[se]c5c(-c6c7ccccc7c(-c7ccc8c(c7)c7ccccc7n8-c7ccccc7)c7ccccc67)cccc5c34)c3ccccc23)cc1]

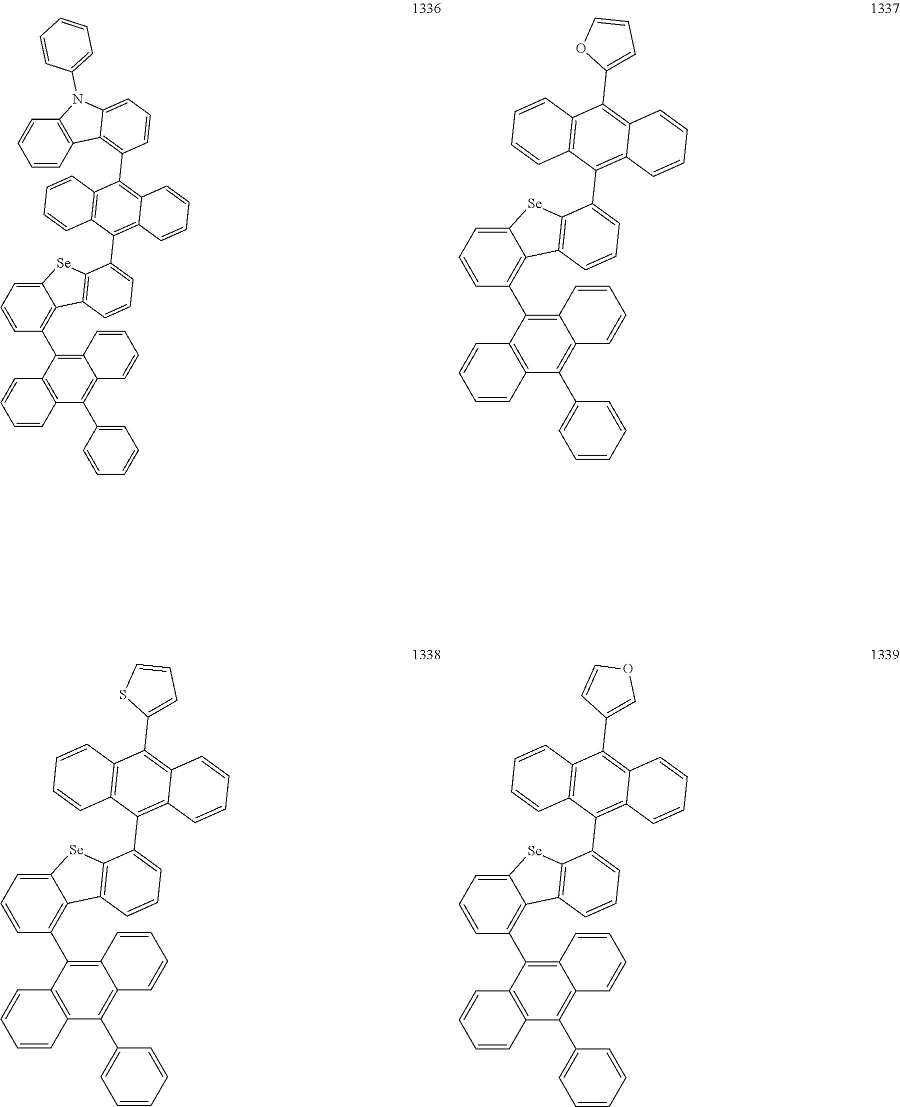 [SMILES: c1ccc(-c2c3ccccc3c(-c3cccc4[se]c5c(-c6c7ccccc7c(-c7cccc8c7c7ccccc7n8-c7ccccc7)c7ccccc67)cccc5c34)c3ccccc23)cc1 ; c1ccc(-c2c3ccccc3c(-c3cccc4[se]c5c(-c6c7ccccc7c(-c7ccco7)c7ccccc67)cccc5c34)c3ccccc23)cc1 ; c1ccc(-c2c3ccccc3c(-c3cccc4[se]c5c(-c6c7ccccc7c(-c7cccs7)c7ccccc67)cccc5c34)c3ccccc23)cc1 ; c1ccc(-c2c3ccccc3c(-c3cccc4[se]c5c(-c6c7ccccc7c(-c7ccoc7)c7ccccc67)cccc5c34)c3ccccc23)cc1]

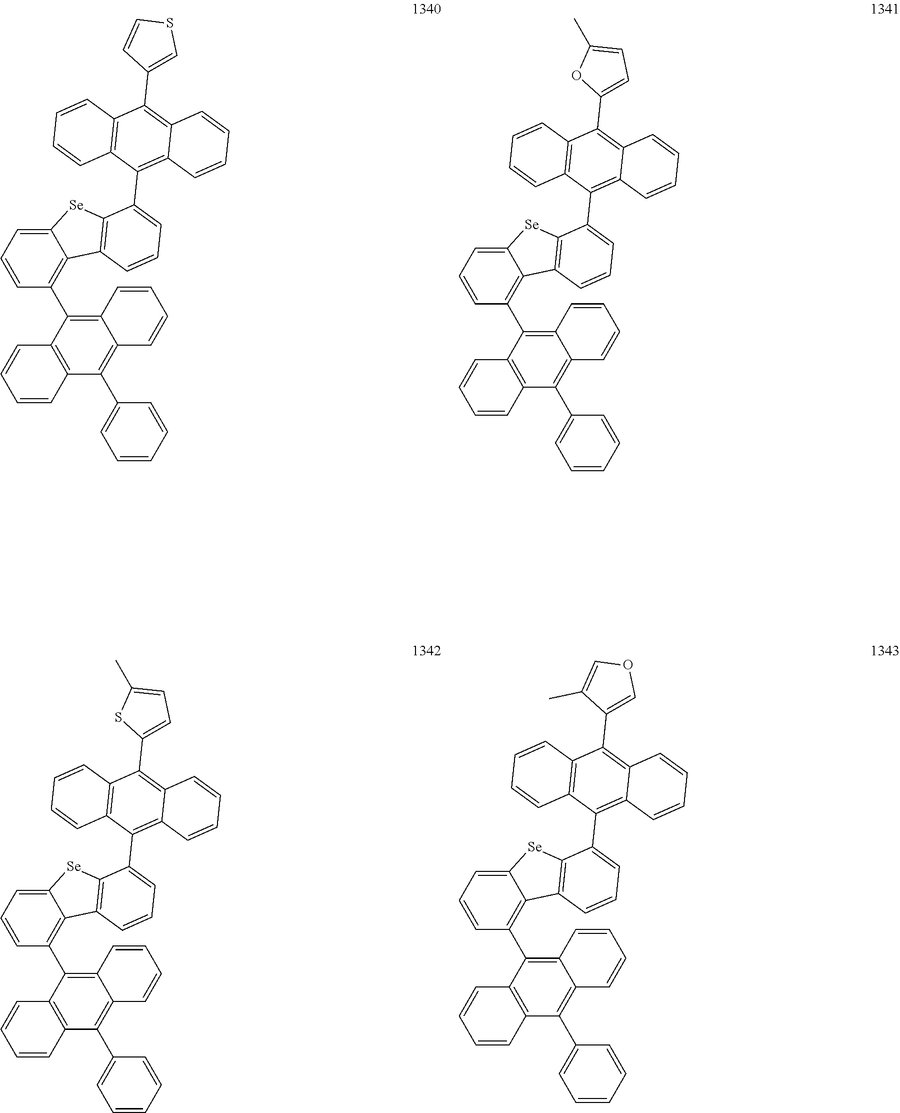 [SMILES: c1ccc(-c2c3ccccc3c(-c3cccc4[se]c5c(-c6c7ccccc7c(-c7ccsc7)c7ccccc67)cccc5c34)c3ccccc23)cc1 ; Cc1ccc(-c2c3ccccc3c(-c3cccc4c3[se]c3cccc(-c5c6ccccc6c(-c6ccccc6)c6ccccc56)c34)c3ccccc23)o1 ; Cc1ccc(-c2c3ccccc3c(-c3cccc4c3[se]c3cccc(-c5c6ccccc6c(-c6ccccc6)c6ccccc56)c34)c3ccccc23)s1 ; Cc1cocc1-c1c2ccccc2c(-c2cccc3c2[se]c2cccc(-c4c5ccccc5c(-c5ccccc5)c5ccccc45)c23)c2ccccc12]

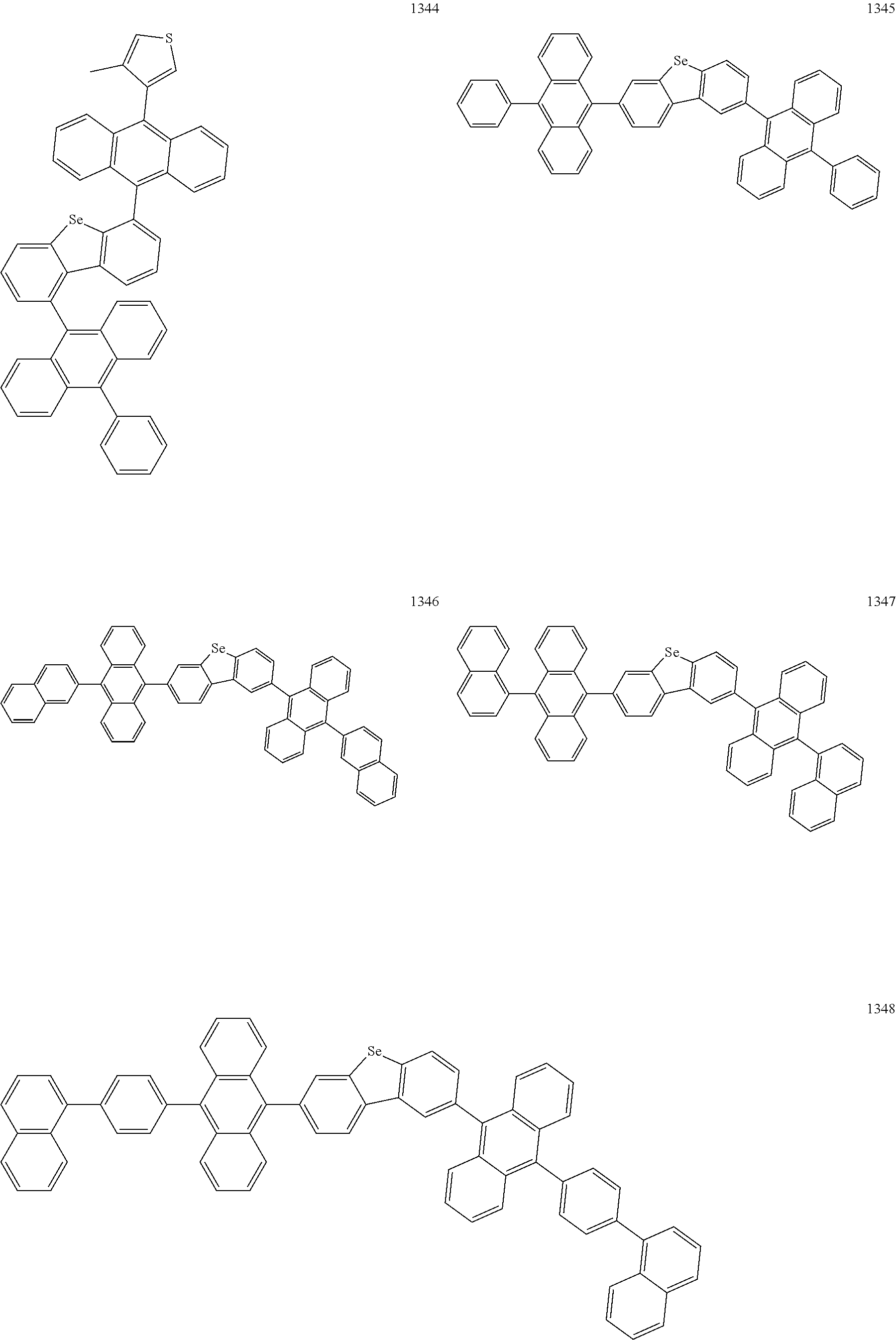

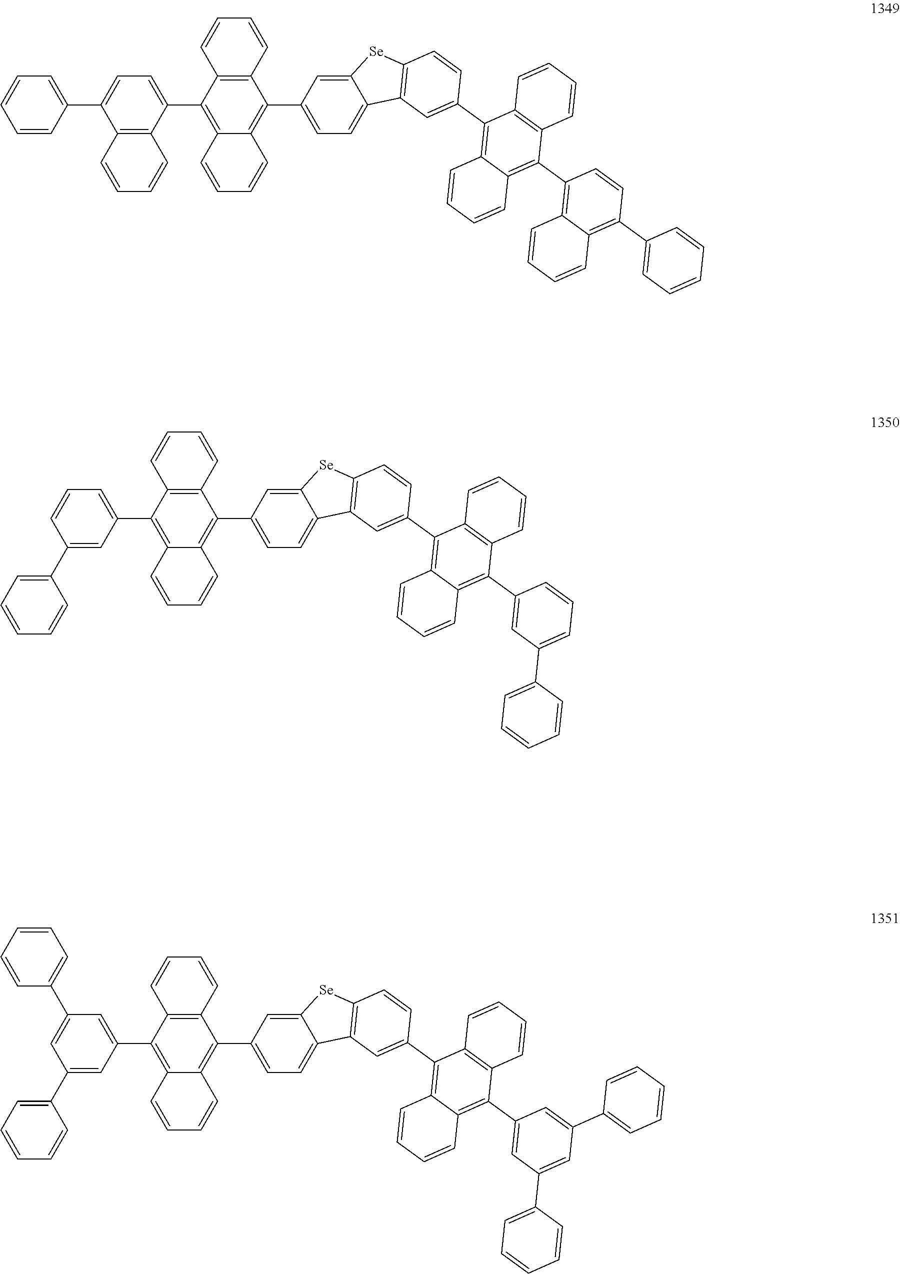 [SMILES: c1ccc(-c2ccc(-c3c4ccccc4c(-c4ccc5c(c4)[se]c4ccc(-c6c7ccccc7c(-c7ccc(-c8ccccc8)c8ccccc78)c7ccccc67)cc45)c4ccccc34)c3ccccc23)cc1 ; c1ccc(-c2cccc(-c3c4ccccc4c(-c4ccc5c(c4)[se]c4ccc(-c6c7ccccc7c(-c7cccc(-c8ccccc8)c7)c7ccccc67)cc45)c4ccccc34)c2)cc1 ; c1ccc(-c2cc(-c3ccccc3)cc(-c3c4ccccc4c(-c4ccc5c(c4)[se]c4ccc(-c6c7ccccc7c(-c7cc(-c8ccccc8)cc(-c8ccccc8)c7)c7ccccc67)cc45)c4ccccc34)c2)cc1]

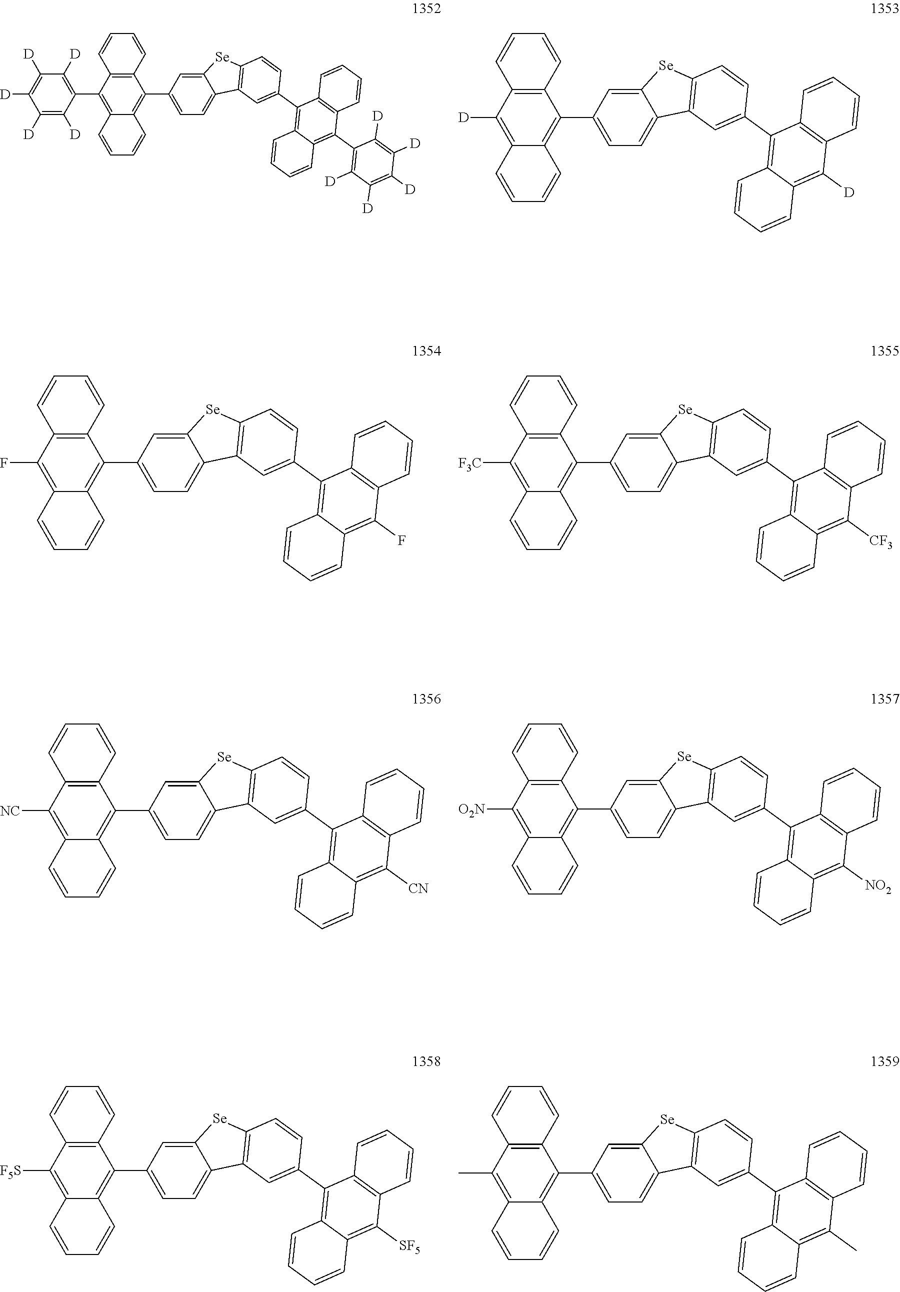

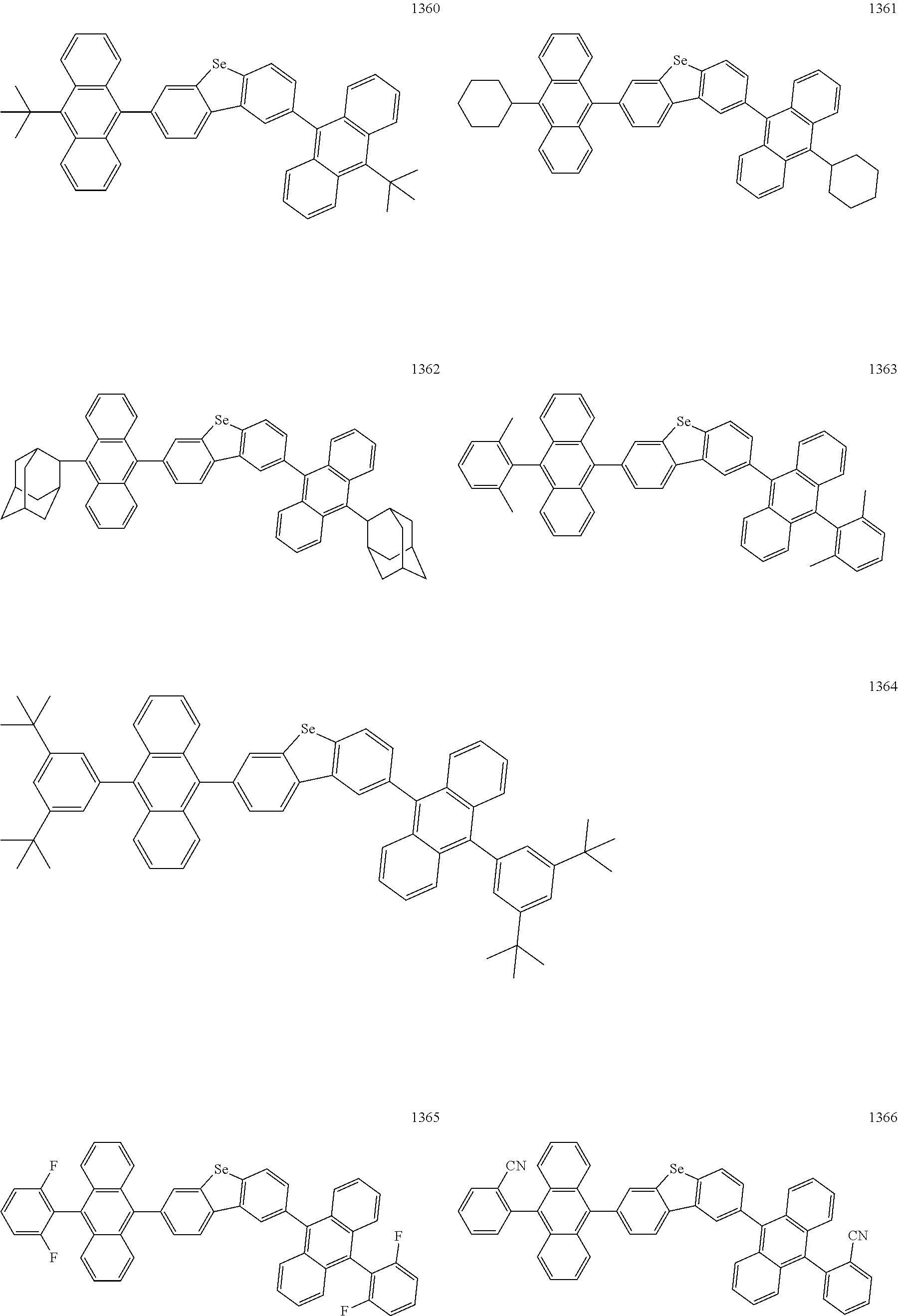

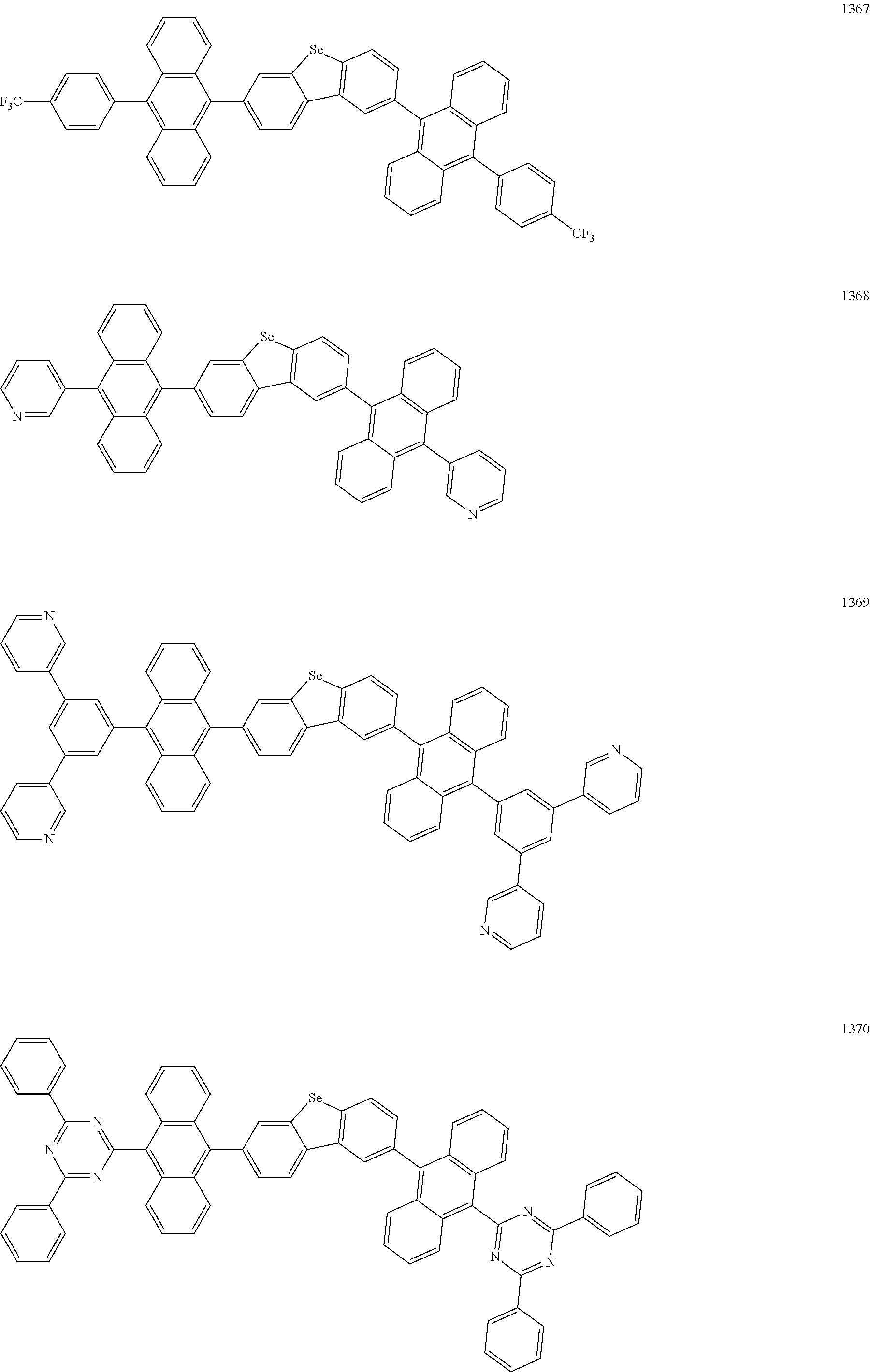 [SMILES: FC(F)(F)c1ccc(-c2c3ccccc3c(-c3ccc4c(c3)[se]c3ccc(-c5c6ccccc6c(-c6ccc(C(F)(F)F)cc6)c6ccccc56)cc34)c3ccccc23)cc1 ; c1cncc(-c2c3ccccc3c(-c3ccc4c(c3)[se]c3ccc(-c5c6ccccc6c(-c6cccnc6)c6ccccc56)cc34)c3ccccc23)c1 ; c1cncc(-c2cc(-c3cccnc3)cc(-c3c4ccccc4c(-c4ccc5c(c4)[se]c4ccc(-c6c7ccccc7c(-c7cc(-c8cccnc8)cc(-c8cccnc8)c7)c7ccccc67)cc45)c4ccccc34)c2)c1 ; c1ccc(-c2nc(-c3ccccc3)nc(-c3c4ccccc4c(-c4ccc5c(c4)[se]c4ccc(-c6c7ccccc7c(-c7nc(-c8ccccc8)nc(-c8ccccc8)n7)c7ccccc67)cc45)c4ccccc34)n2)cc1]

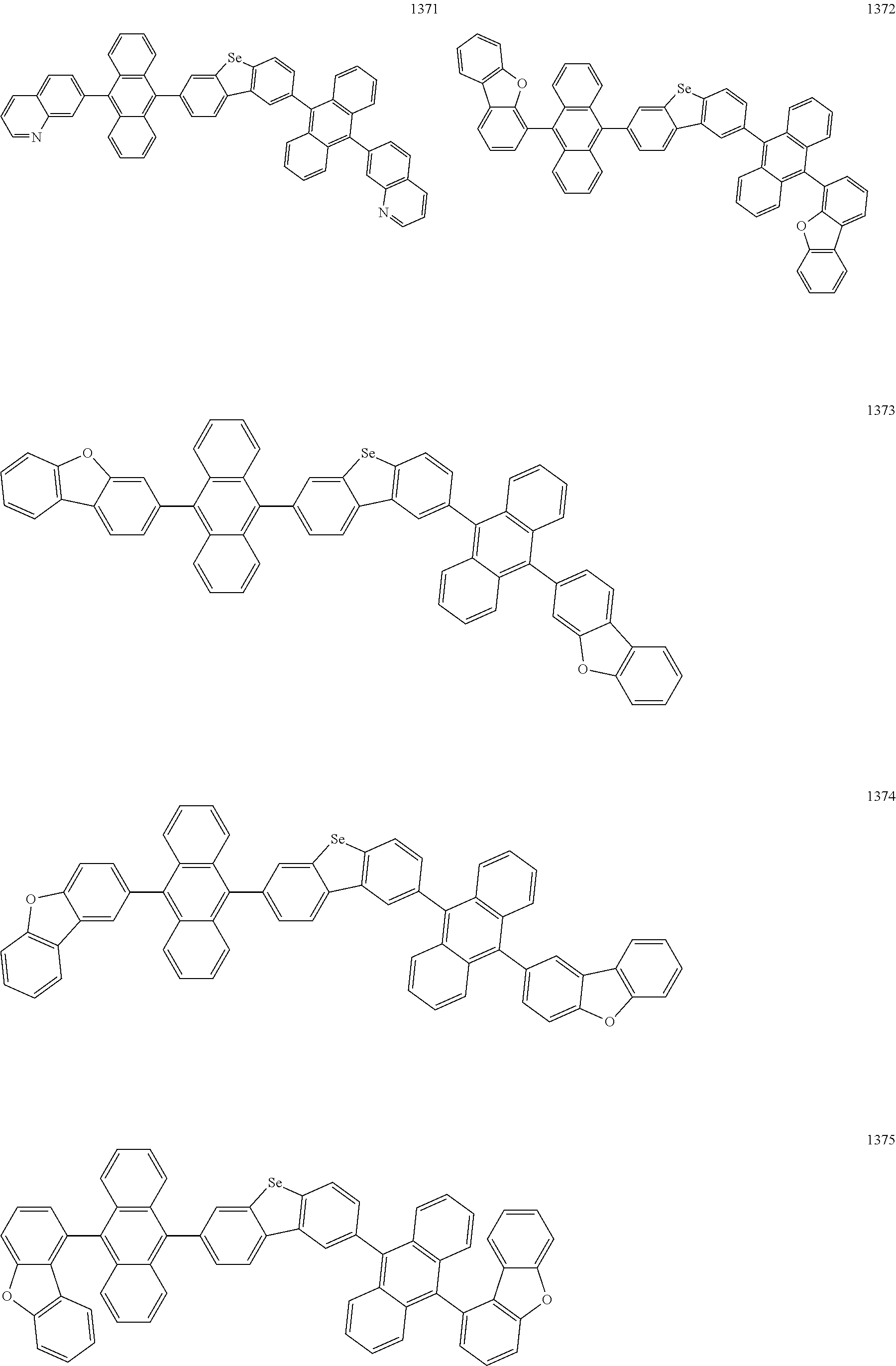

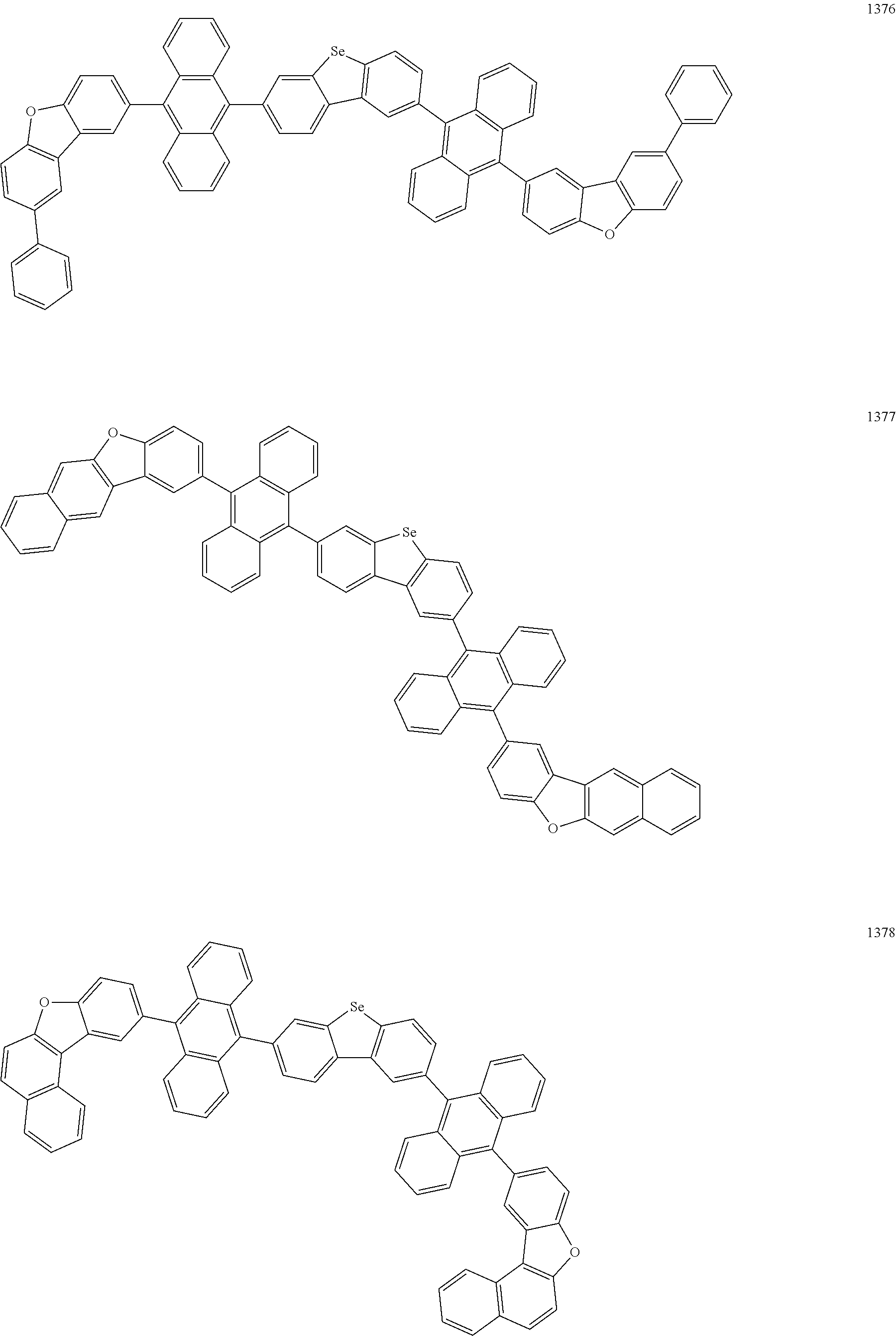 [SMILES: c1ccc(-c2ccc3oc4ccc(-c5c6ccccc6c(-c6ccc7c(c6)[se]c6ccc(-c8c9ccccc9c(-c9ccc%10oc%11ccc(-c%12ccccc%12)cc%11c%10c9)c9ccccc89)cc67)c6ccccc56)cc4c3c2)cc1 ; c1ccc2cc3c(cc2c1)oc1ccc(-c2c4ccccc4c(-c4ccc5c(c4)[se]c4ccc(-c6c7ccccc7c(-c7ccc8oc9cc%10ccccc%10cc9c8c7)c7ccccc67)cc45)c4ccccc24)cc13 ; c1ccc2c(c1)ccc1oc3ccc(-c4c5ccccc5c(-c5ccc6c(c5)[se]c5ccc(-c7c8ccccc8c(-c8ccc9oc%10ccc%11ccccc%11c%10c9c8)c8ccccc78)cc56)c5ccccc45)cc3c12]

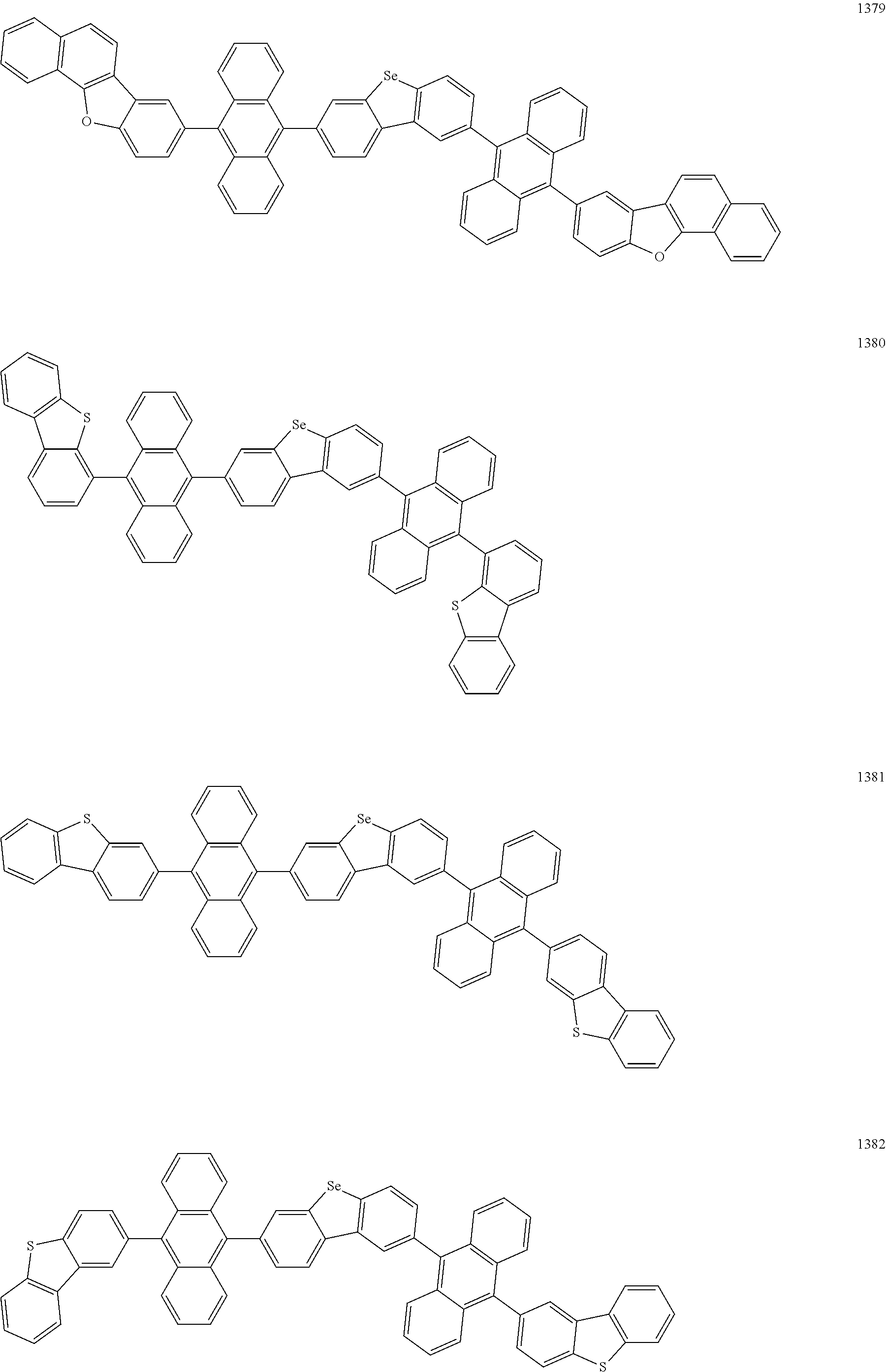 [SMILES: c1ccc2c(c1)ccc1c3cc(-c4c5ccccc5c(-c5ccc6c(c5)[se]c5ccc(-c7c8ccccc8c(-c8ccc9oc%10c%11ccccc%11ccc%10c9c8)c8ccccc78)cc56)c5ccccc45)ccc3oc21 ; c1ccc2c(c1)sc1c(-c3c4ccccc4c(-c4ccc5c(c4)[se]c4ccc(-c6c7ccccc7c(-c7cccc8c7sc7ccccc78)c7ccccc67)cc45)c4ccccc34)cccc12 ; c1ccc2c(c1)sc1cc(-c3c4ccccc4c(-c4ccc5c(c4)[se]c4ccc(-c6c7ccccc7c(-c7ccc8c(c7)sc7ccccc78)c7ccccc67)cc45)c4ccccc34)ccc12 ; c1ccc2c(c1)sc1ccc(-c3c4ccccc4c(-c4ccc5c(c4)[se]c4ccc(-c6c7ccccc7c(-c7ccc8sc9ccccc9c8c7)c7ccccc67)cc45)c4ccccc34)cc12]

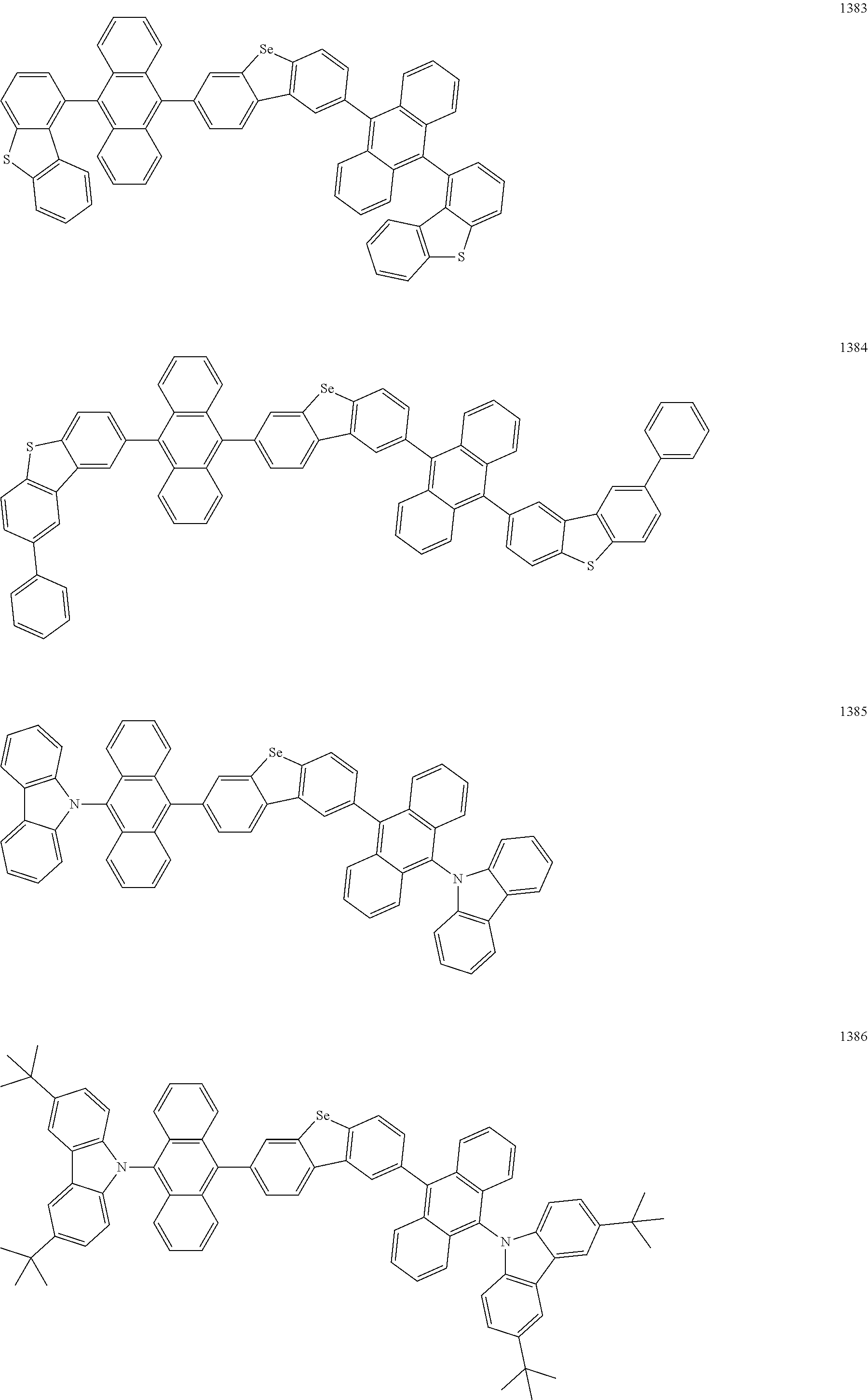

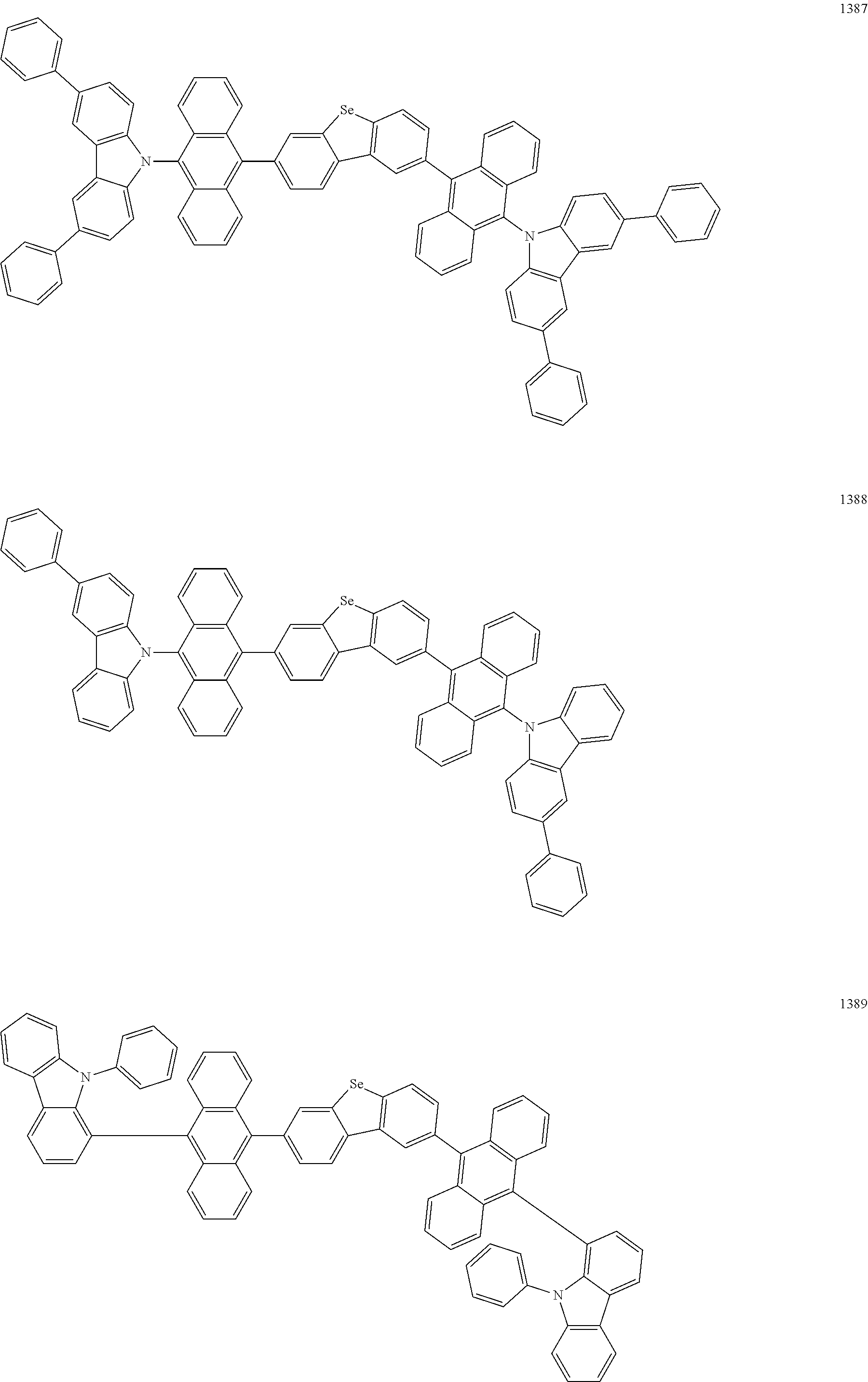 [SMILES: c1ccc(-c2ccc3c(c2)c2cc(-c4ccccc4)ccc2n3-c2c3ccccc3c(-c3ccc4c(c3)[se]c3ccc(-c5c6ccccc6c(-n6c7ccc(-c8ccccc8)cc7c7cc(-c8ccccc8)ccc76)c6ccccc56)cc34)c3ccccc23)cc1 ; c1ccc(-c2ccc3c(c2)c2ccccc2n3-c2c3ccccc3c(-c3ccc4c(c3)[se]c3ccc(-c5c6ccccc6c(-n6c7ccccc7c7cc(-c8ccccc8)ccc76)c6ccccc56)cc34)c3ccccc23)cc1 ; c1ccc(-n2c3ccccc3c3cccc(-c4c5ccccc5c(-c5ccc6c(c5)[se]c5ccc(-c7c8ccccc8c(-c8cccc9c%10ccccc%10n(-c%10ccccc%10)c89)c8ccccc78)cc56)c5ccccc45)c32)cc1]

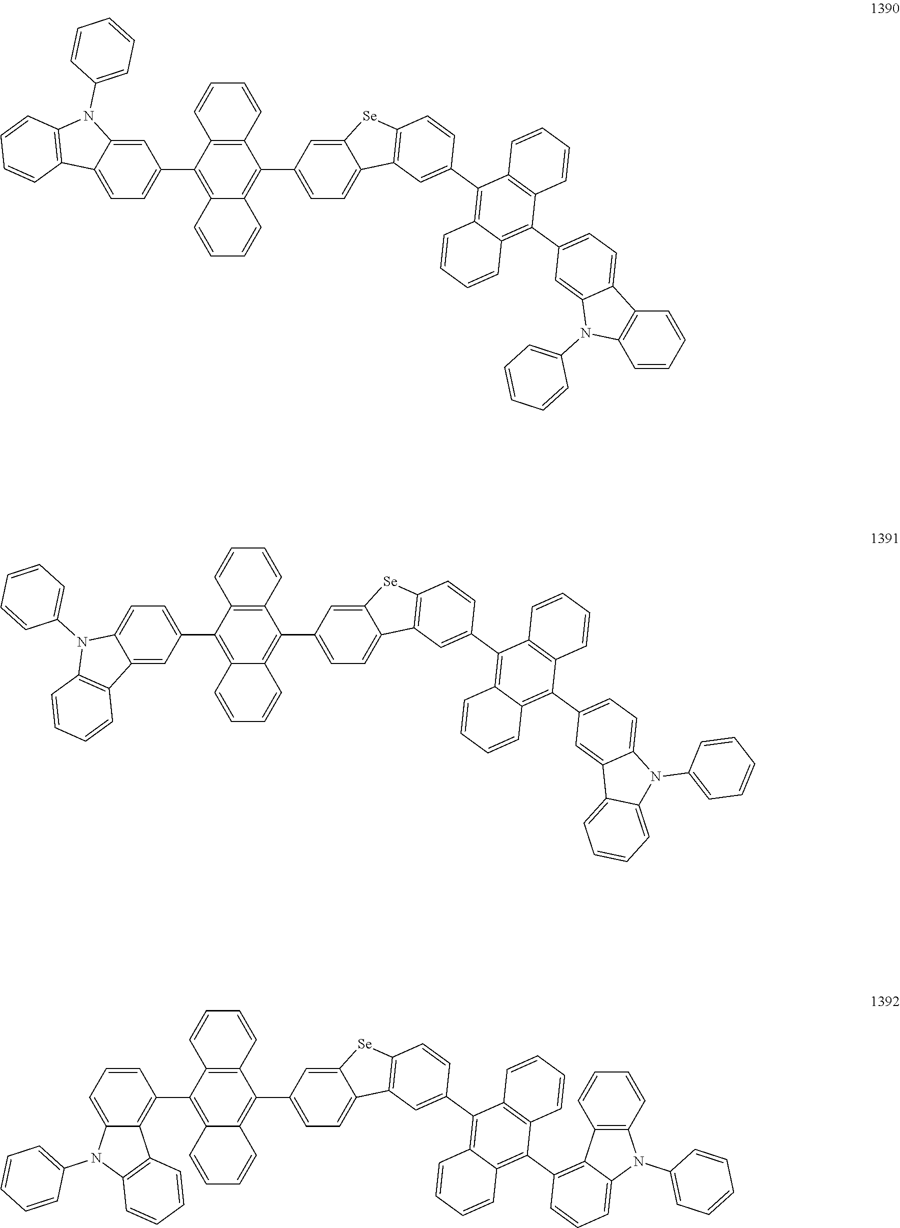 [SMILES: c1ccc(-n2c3ccccc3c3ccc(-c4c5ccccc5c(-c5ccc6c(c5)[se]c5ccc(-c7c8ccccc8c(-c8ccc9c%10ccccc%10n(-c%10ccccc%10)c9c8)c8ccccc78)cc56)c5ccccc45)cc32)cc1 ; c1ccc(-n2c3ccccc3c3cc(-c4c5ccccc5c(-c5ccc6c(c5)[se]c5ccc(-c7c8ccccc8c(-c8ccc9c(c8)c8ccccc8n9-c8ccccc8)c8ccccc78)cc56)c5ccccc45)ccc32)cc1 ; c1ccc(-n2c3ccccc3c3c(-c4c5ccccc5c(-c5ccc6c(c5)[se]c5ccc(-c7c8ccccc8c(-c8cccc9c8c8ccccc8n9-c8ccccc8)c8ccccc78)cc56)c5ccccc45)cccc32)cc1]

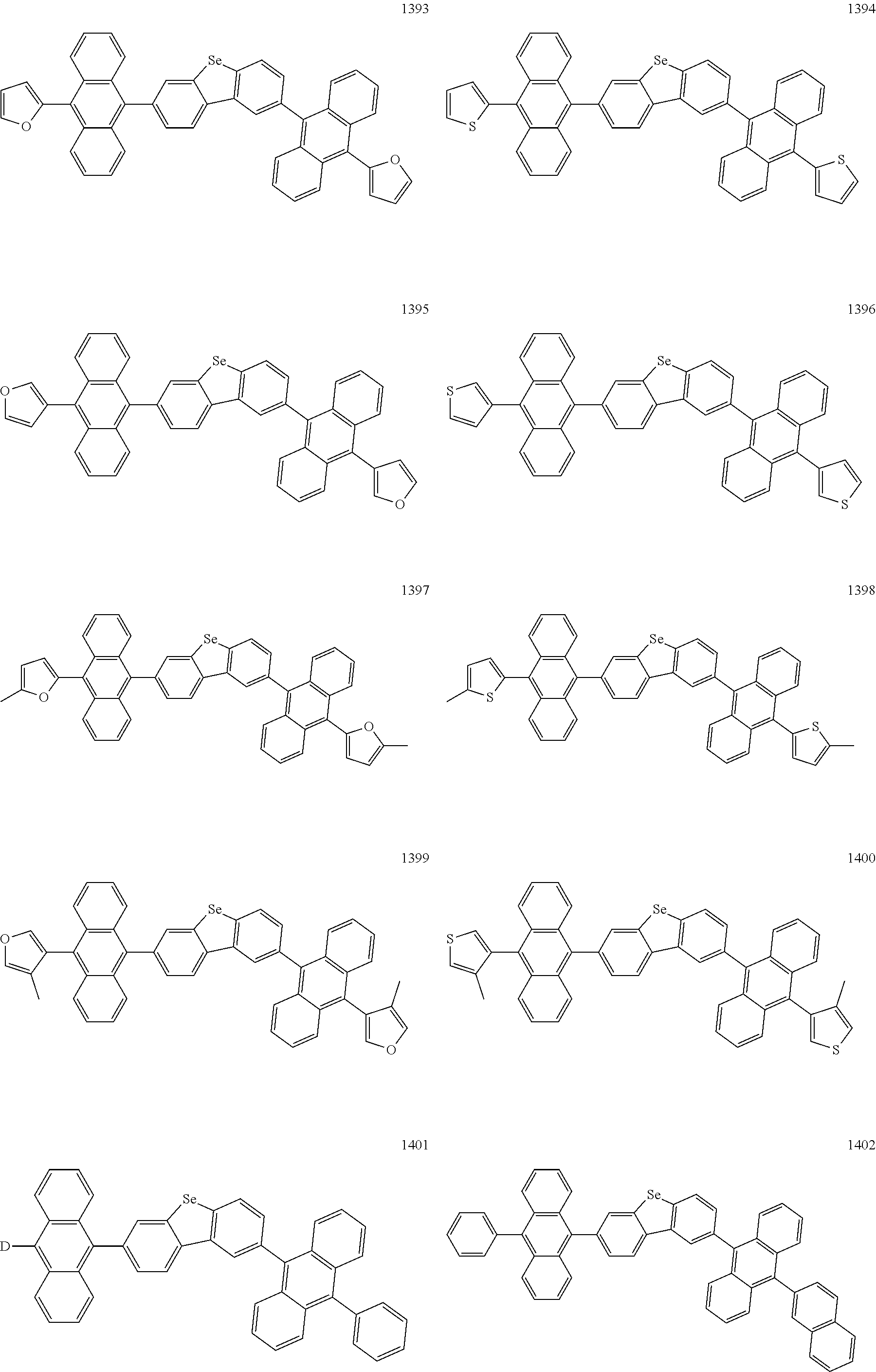

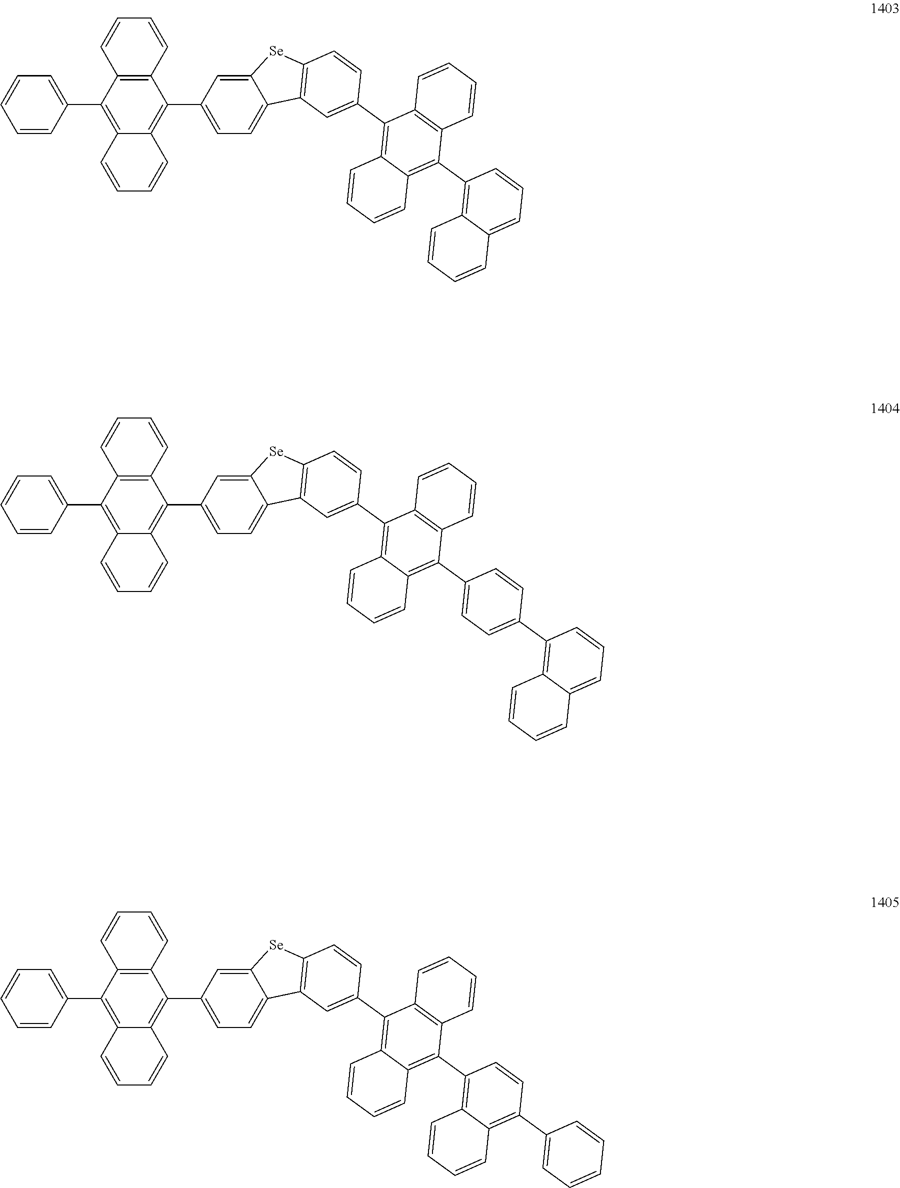 [SMILES: c1ccc(-c2c3ccccc3c(-c3ccc4c(c3)[se]c3ccc(-c5c6ccccc6c(-c6cccc7ccccc67)c6ccccc56)cc34)c3ccccc23)cc1 ; c1ccc(-c2c3ccccc3c(-c3ccc4c(c3)[se]c3ccc(-c5c6ccccc6c(-c6ccc(-c7cccc8ccccc78)cc6)c6ccccc56)cc34)c3ccccc23)cc1 ; c1ccc(-c2ccc(-c3c4ccccc4c(-c4ccc5[se]c6cc(-c7c8ccccc8c(-c8ccccc8)c8ccccc78)ccc6c5c4)c4ccccc34)c3ccccc23)cc1]

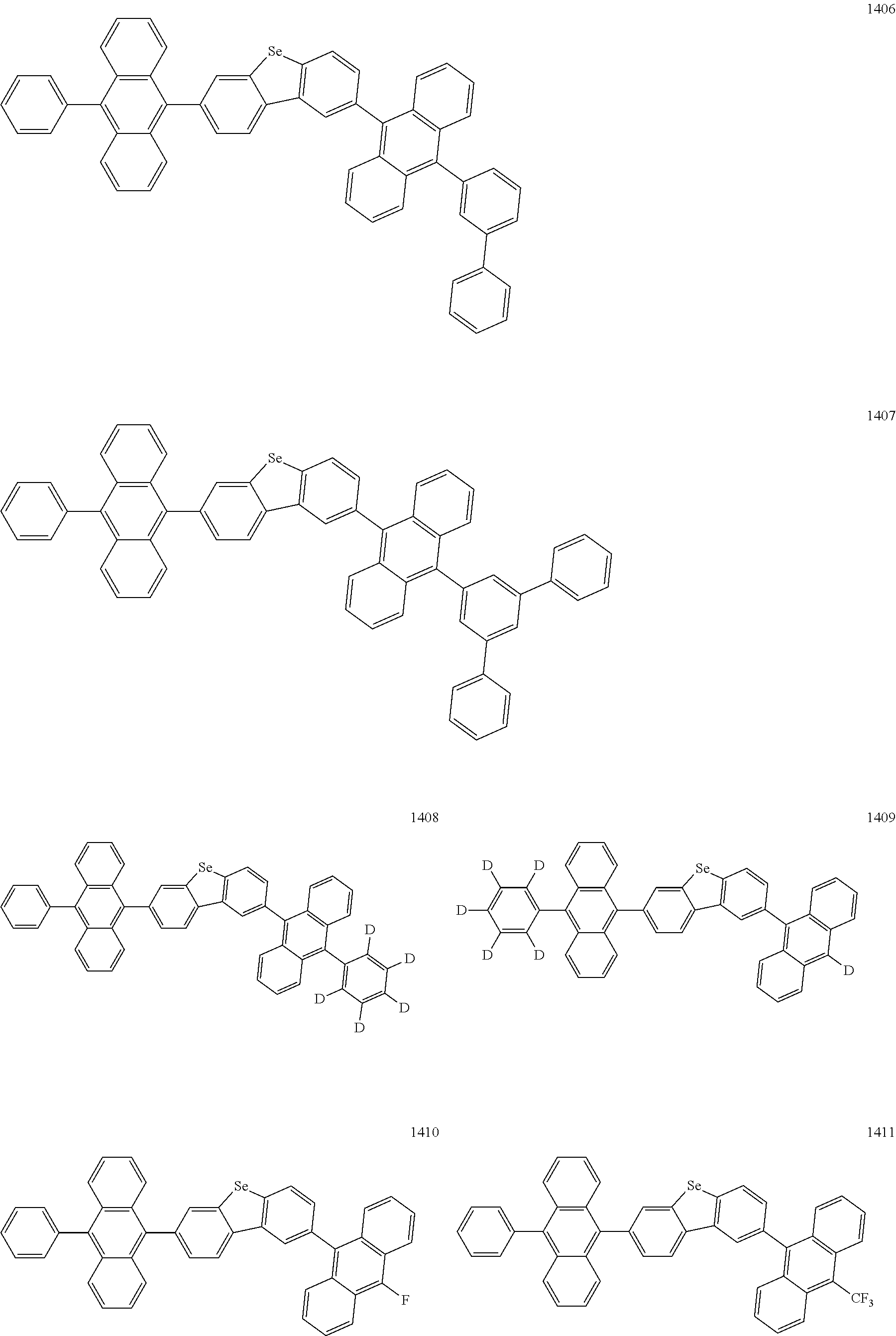

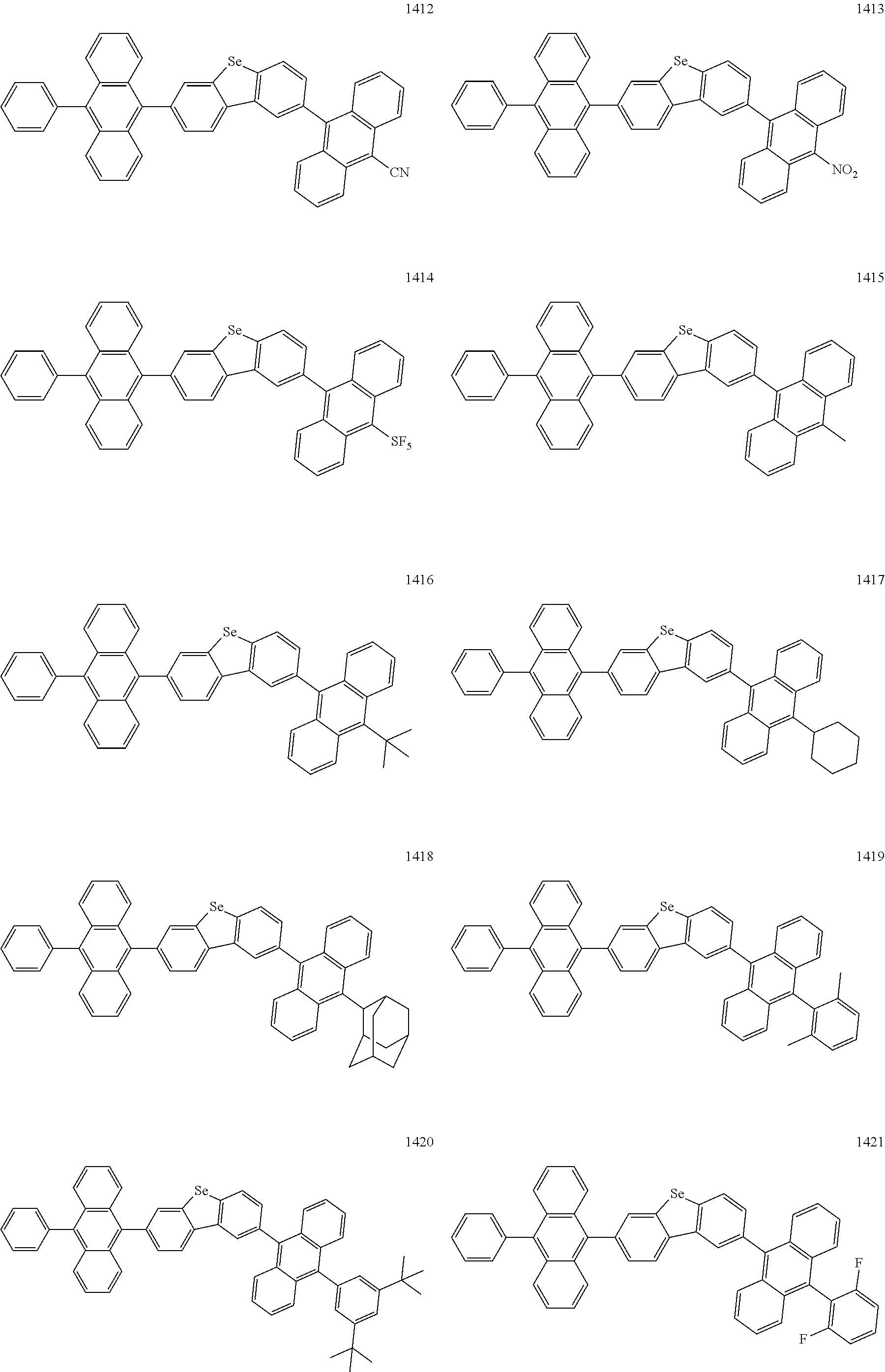

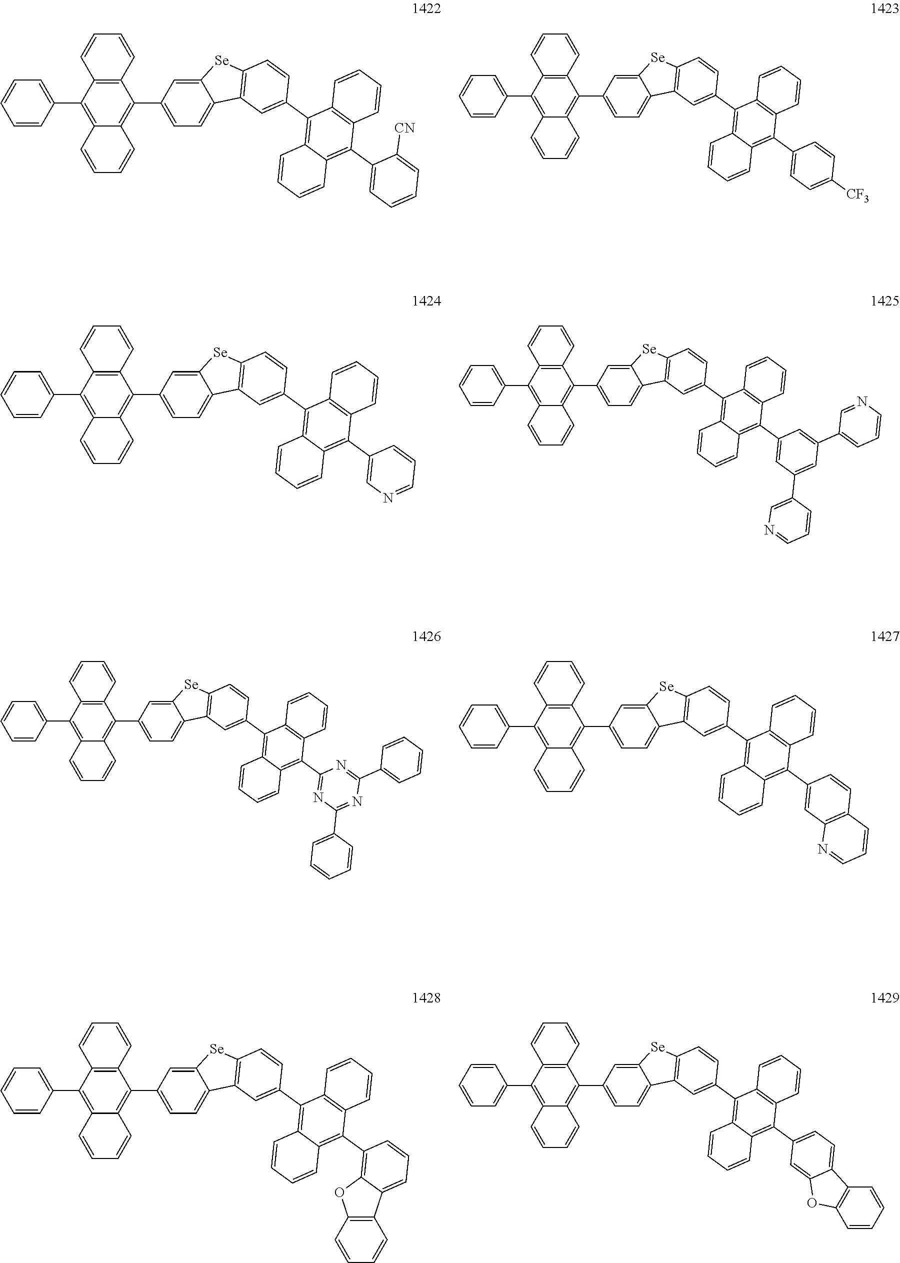

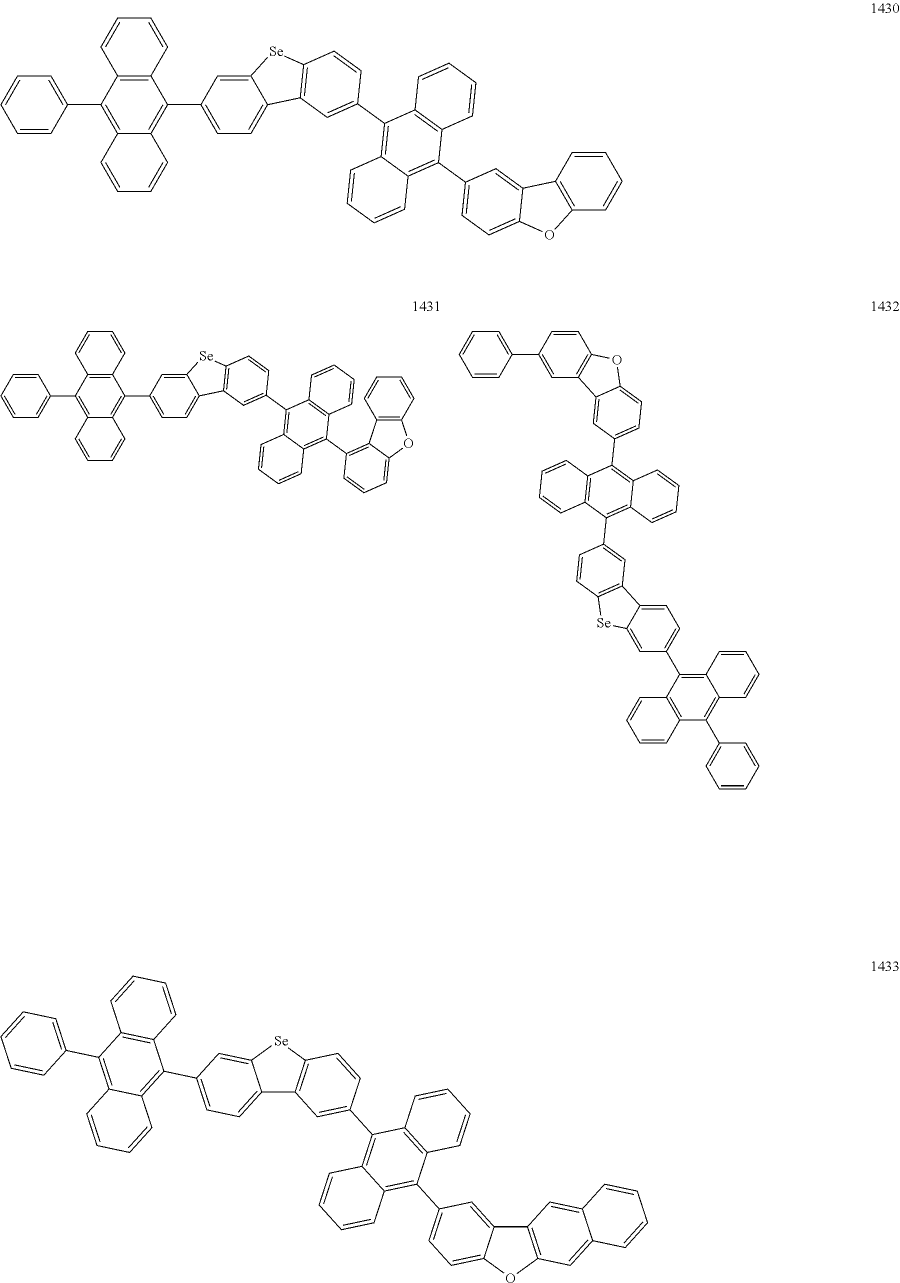 [SMILES: c1ccc(-c2c3ccccc3c(-c3ccc4c(c3)[se]c3ccc(-c5c6ccccc6c(-c6ccc7oc8ccccc8c7c6)c6ccccc56)cc34)c3ccccc23)cc1 ; c1ccc(-c2c3ccccc3c(-c3ccc4c(c3)[se]c3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7[se]c8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)[se]c3ccc(-c5c6ccccc6c(-c6ccc7oc8cc9ccccc9cc8c7c6)c6ccccc56)cc34)c3ccccc23)cc1]

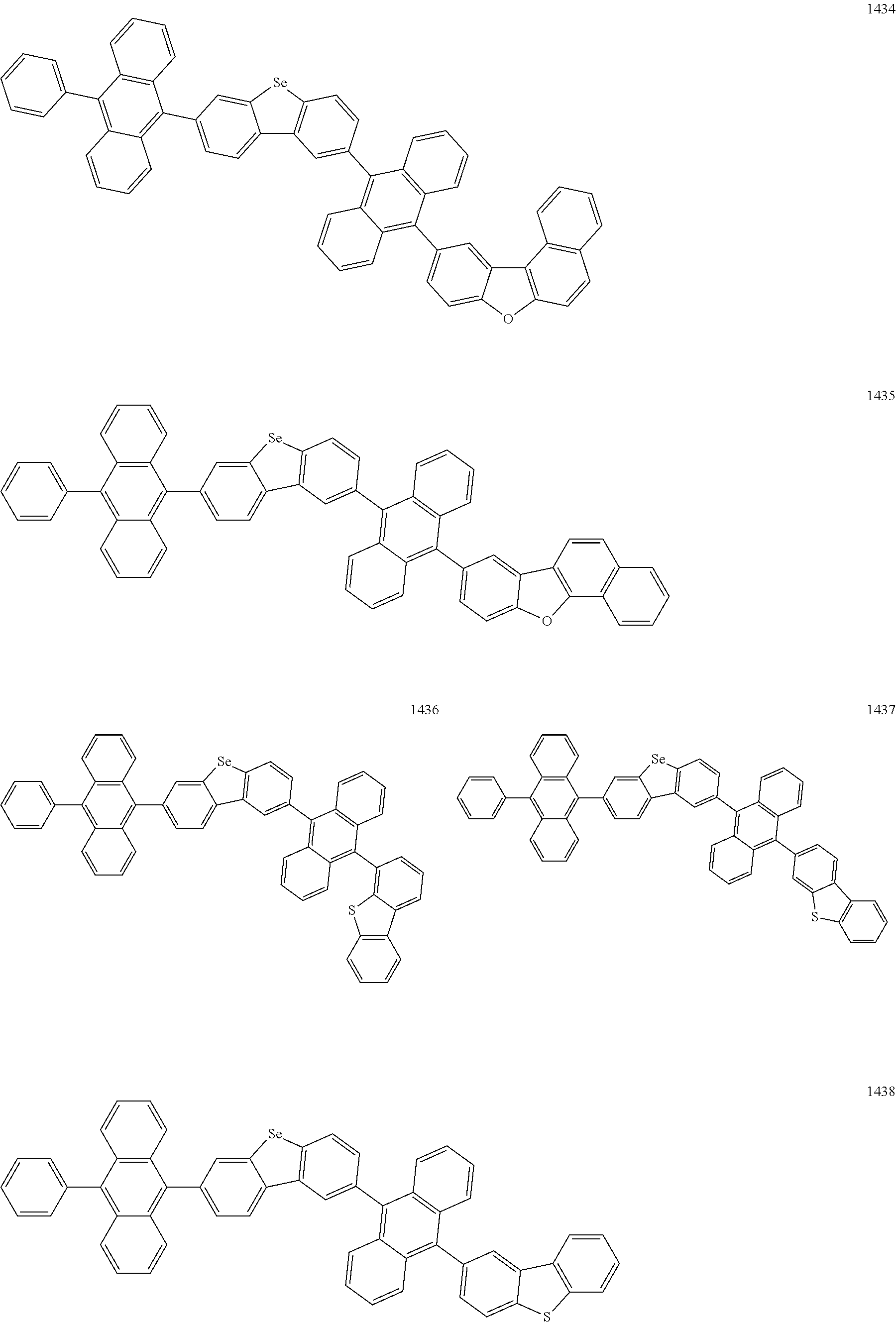

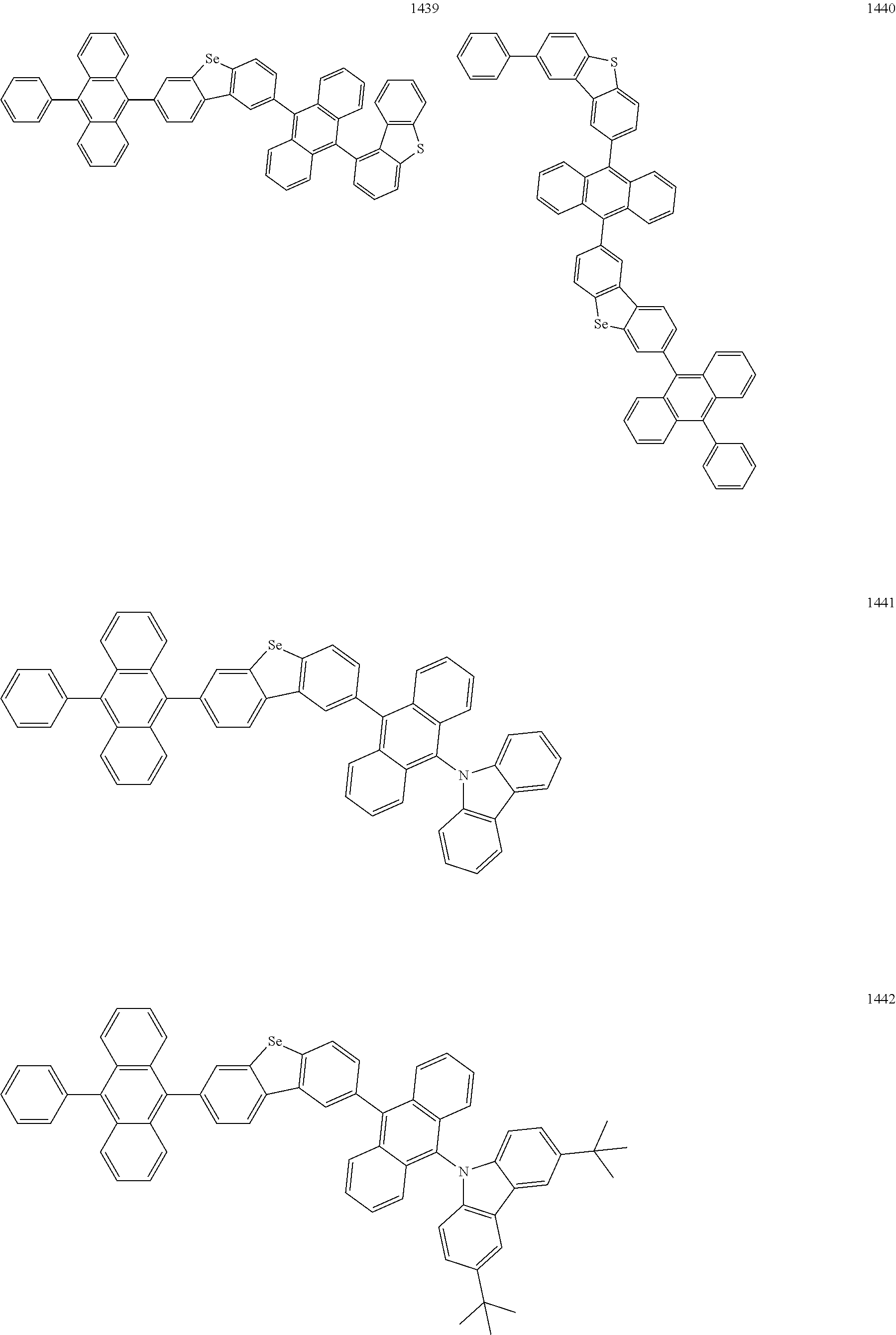 [SMILES: c1ccc(-c2c3ccccc3c(-c3ccc4c(c3)[se]c3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7[se]c8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)[se]c3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3[se]c4cc(-c5c6ccccc6c(-c6ccccc6)c6ccccc56)ccc4c3c2)c2ccccc12]

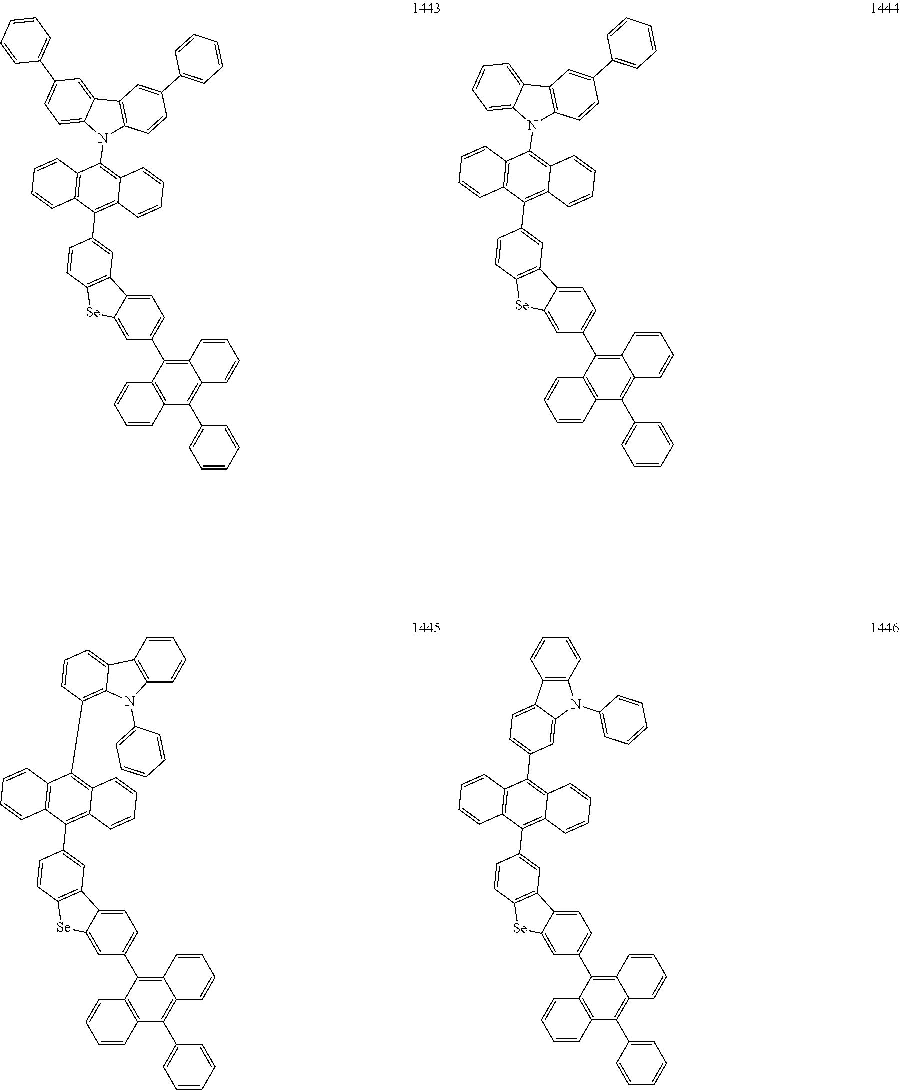 [SMILES: c1ccc(-c2ccc3c(c2)c2cc(-c4ccccc4)ccc2n3-c2c3ccccc3c(-c3ccc4[se]c5cc(-c6c7ccccc7c(-c7ccccc7)c7ccccc67)ccc5c4c3)c3ccccc23)cc1 ; c1ccc(-c2ccc3c(c2)c2ccccc2n3-c2c3ccccc3c(-c3ccc4[se]c5cc(-c6c7ccccc7c(-c7ccccc7)c7ccccc67)ccc5c4c3)c3ccccc23)cc1 ; c1ccc(-c2c3ccccc3c(-c3ccc4c(c3)[se]c3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)[se]c3ccc(-c5c6ccccc6c(-c6ccc7c8ccccc8n(-c8ccccc8)c7c6)c6ccccc56)cc34)c3ccccc23)cc1]

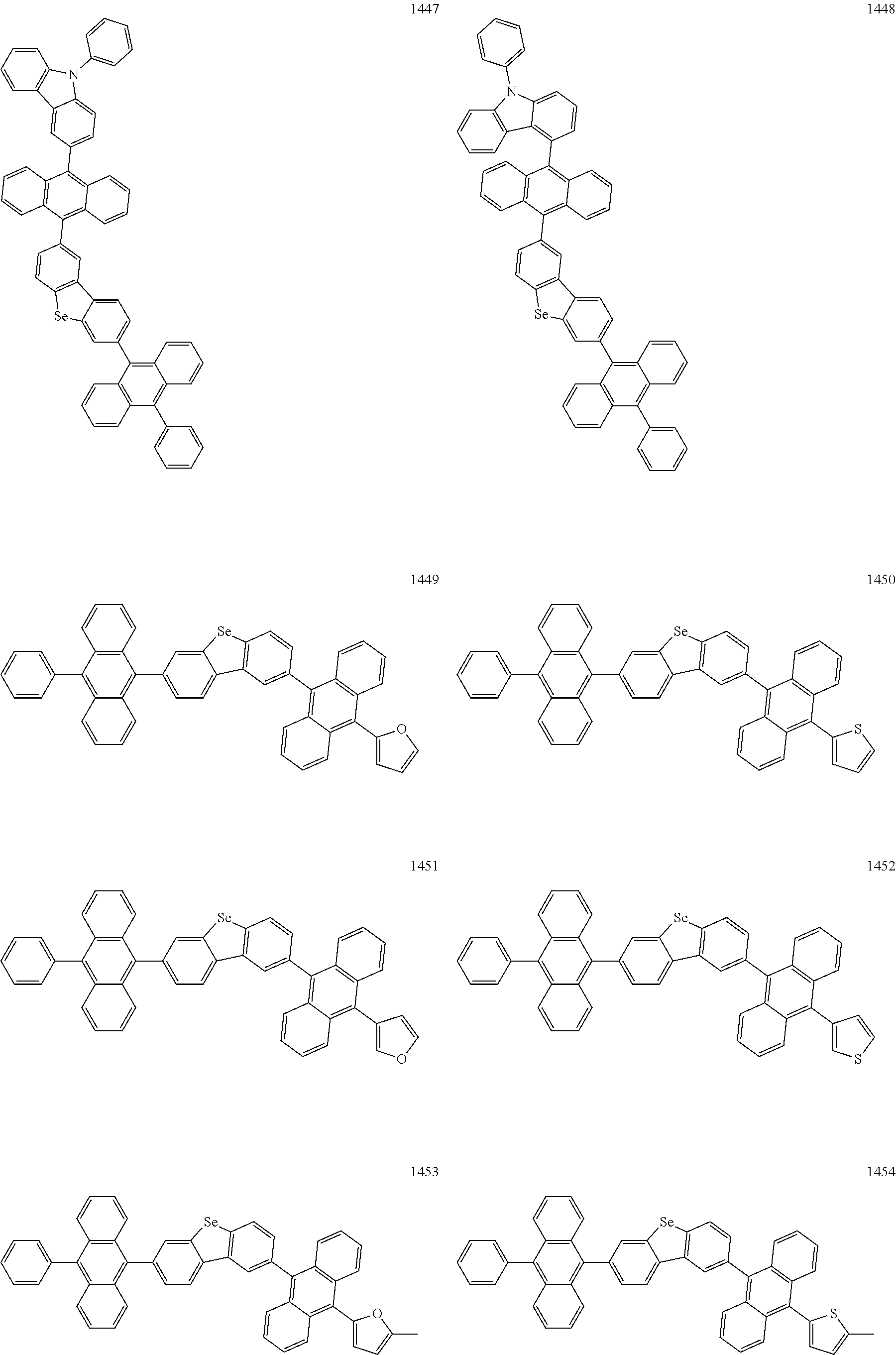

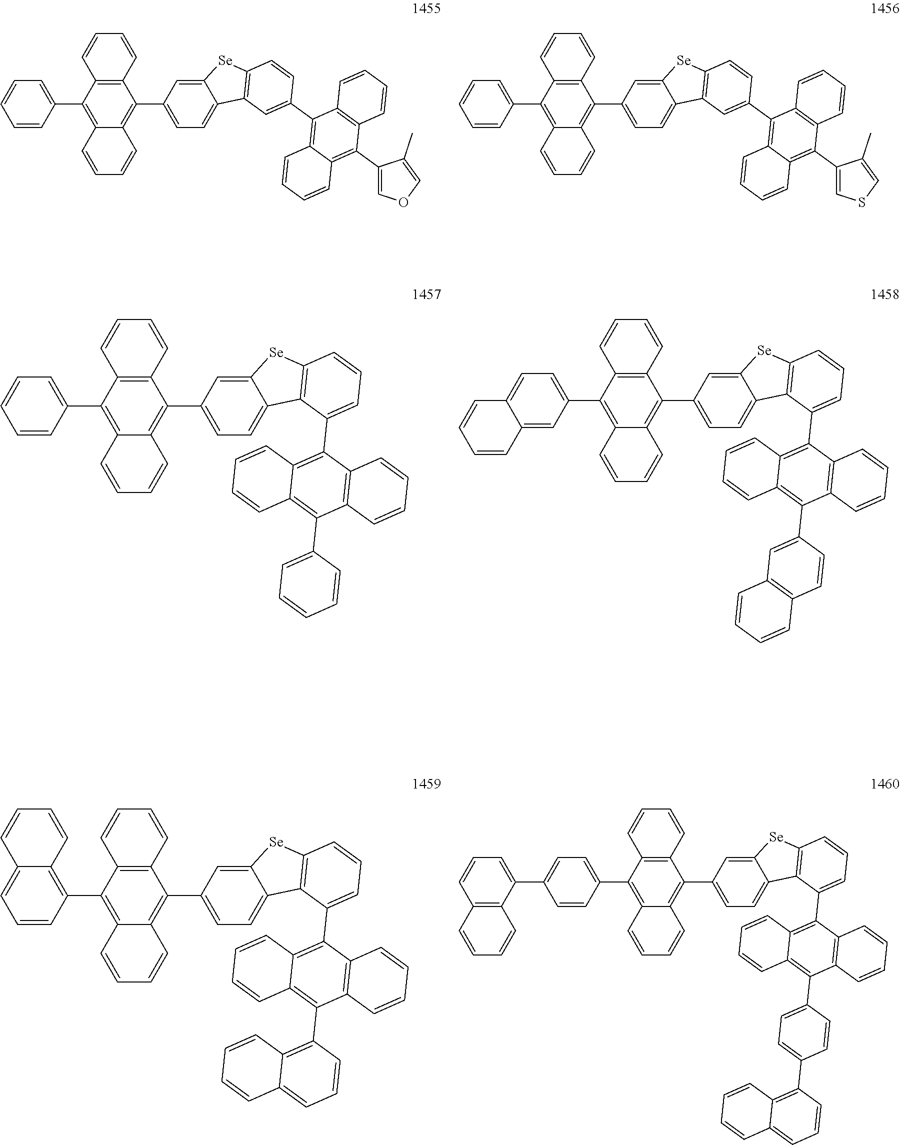

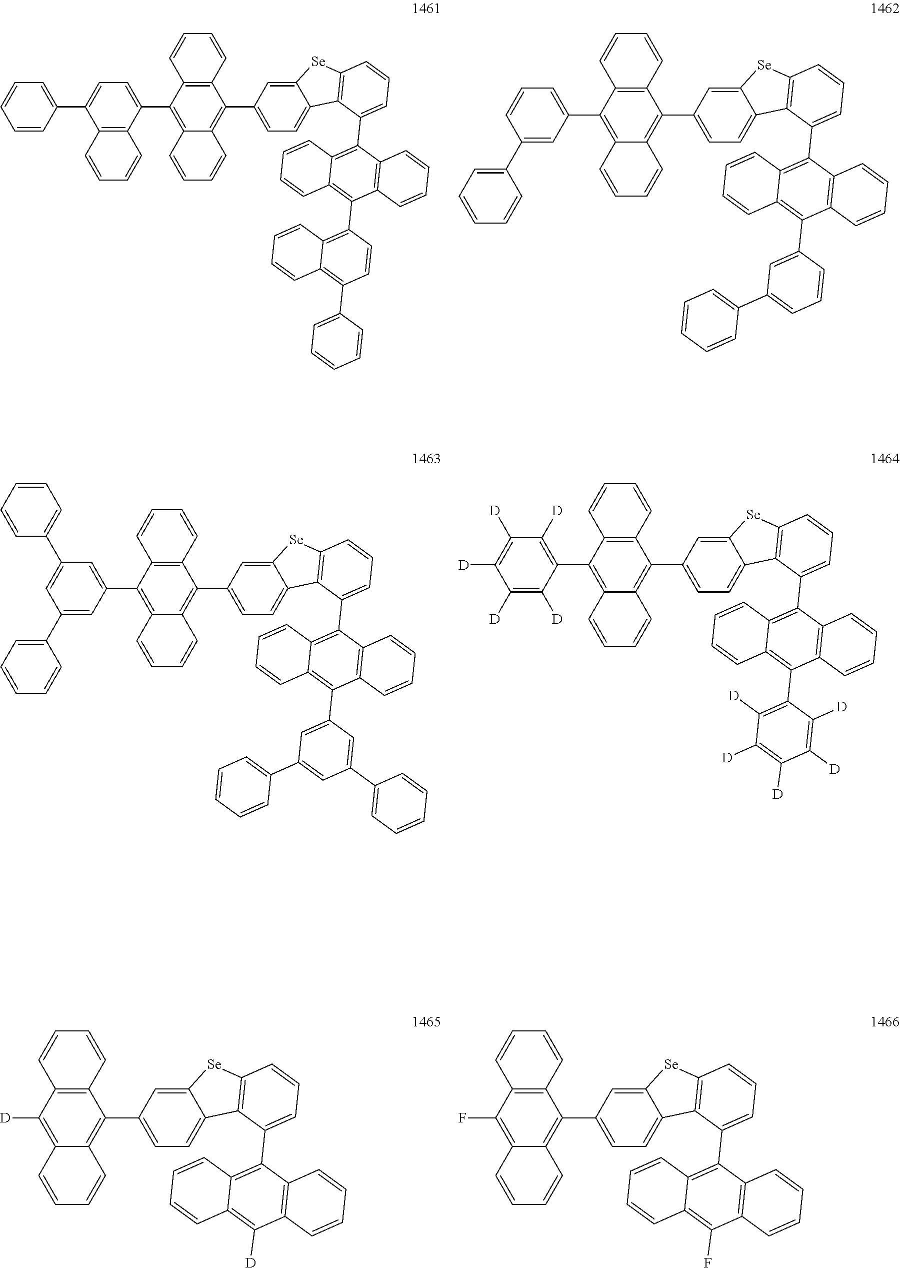

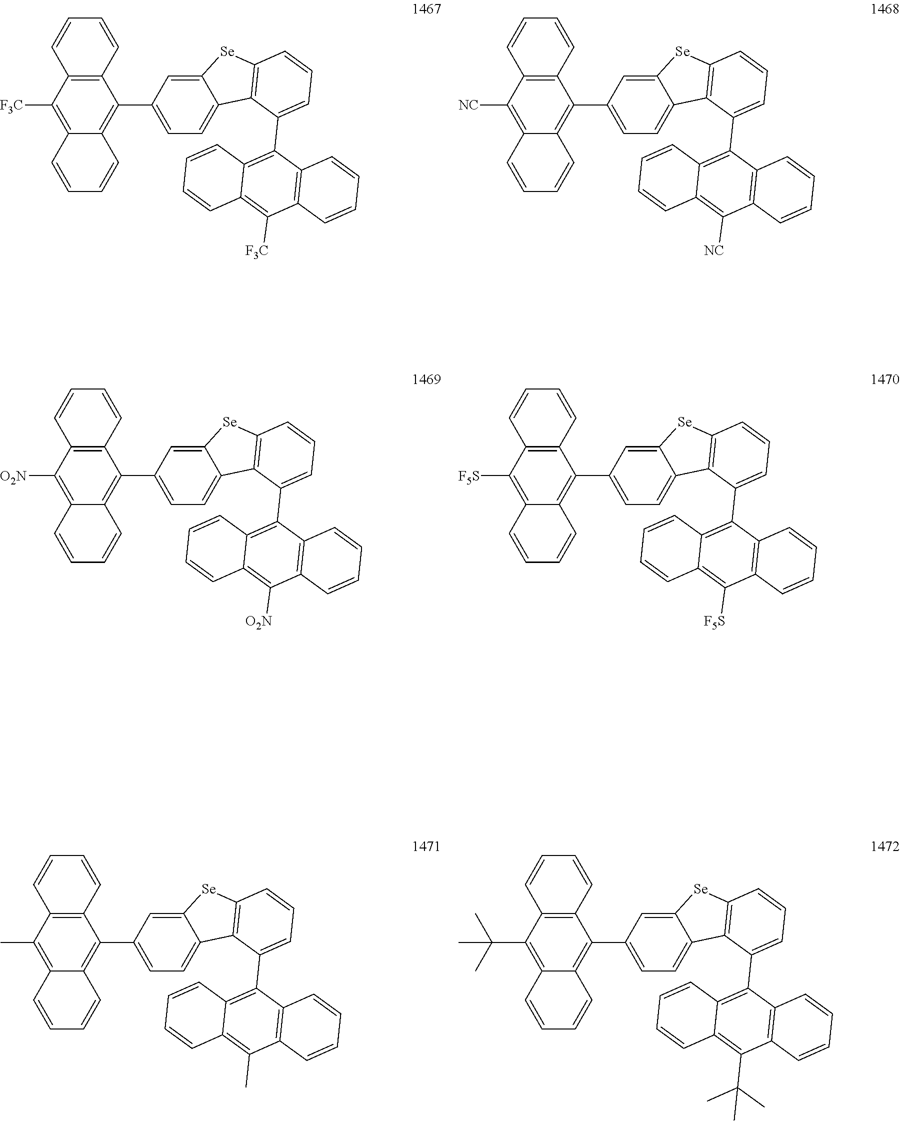

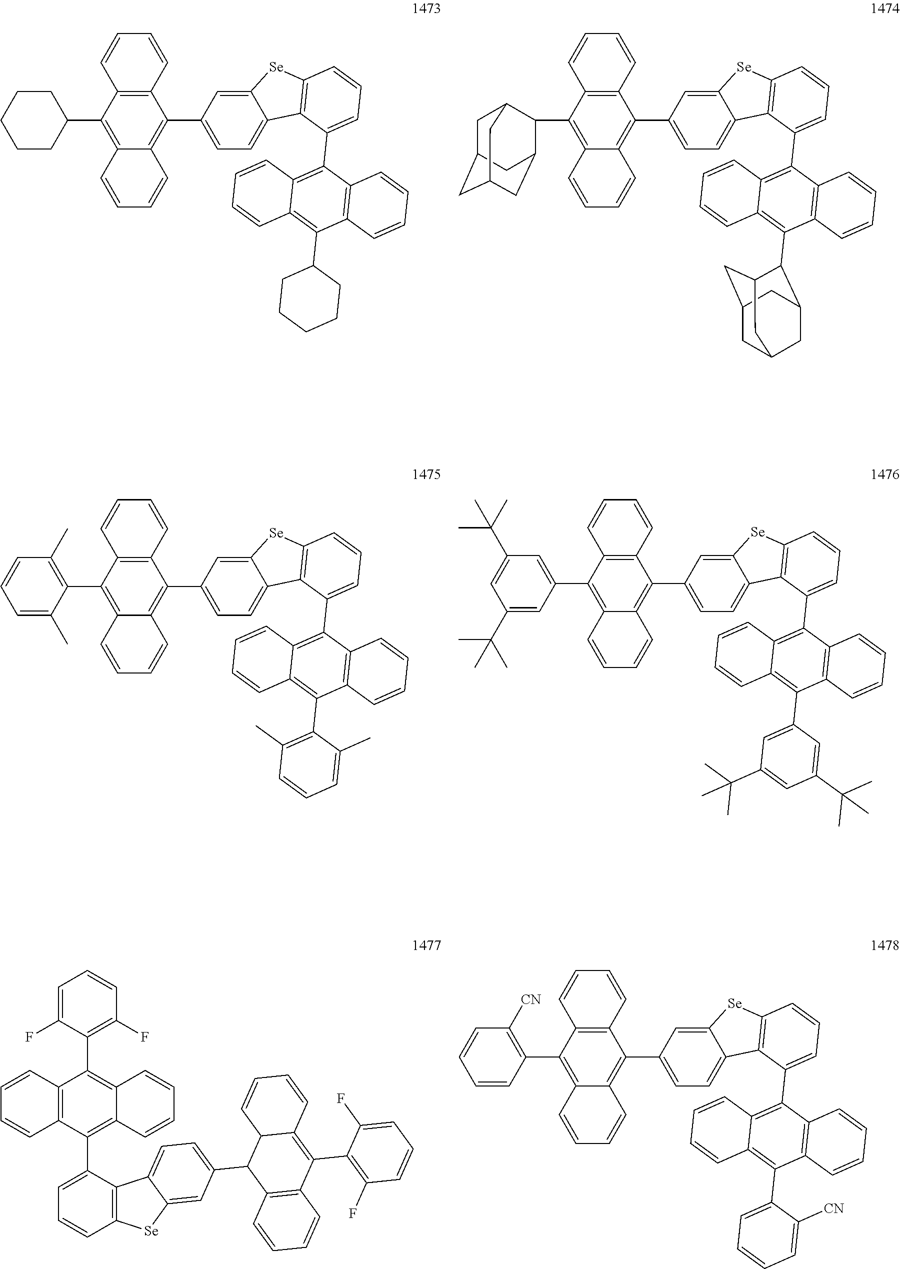

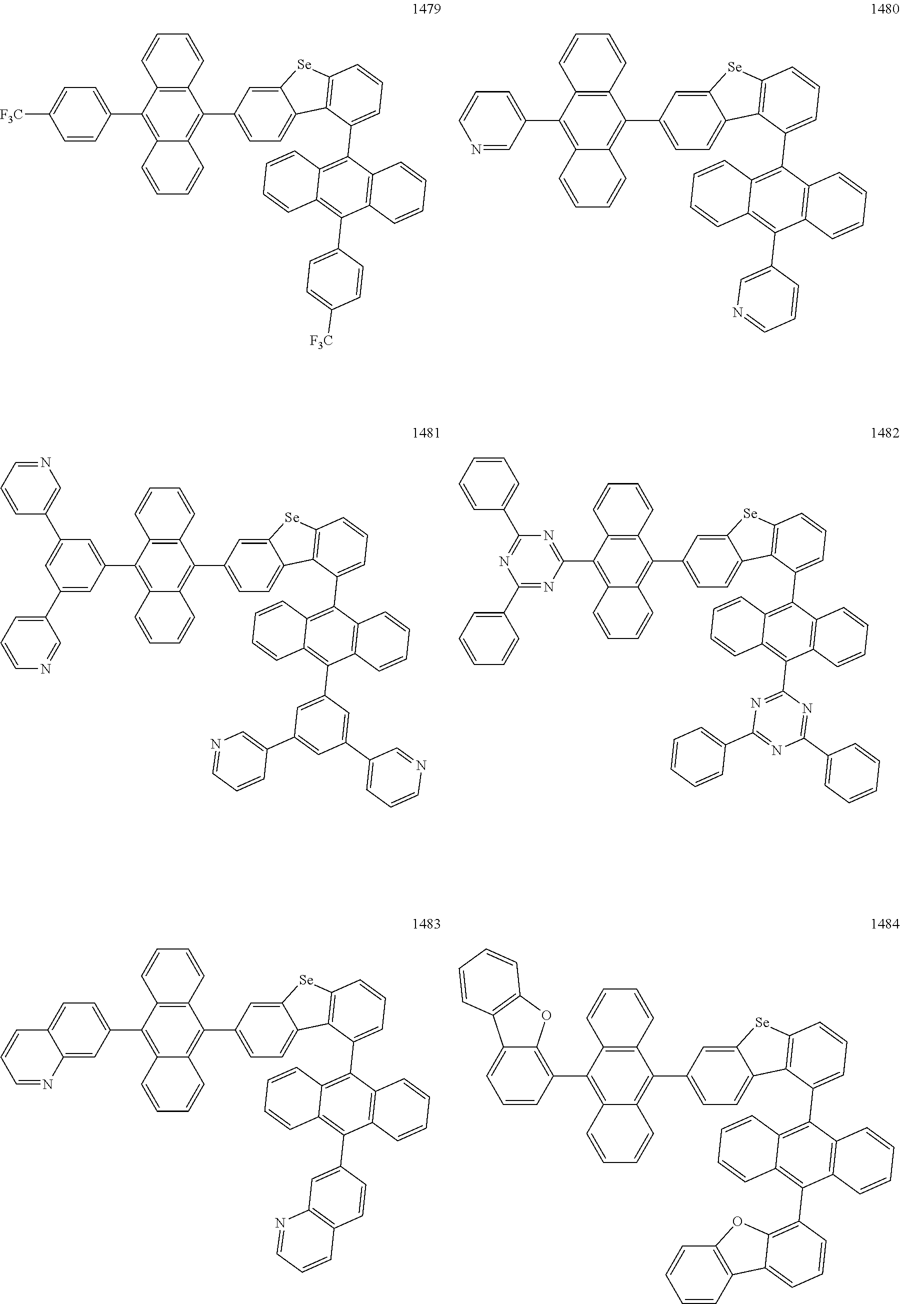

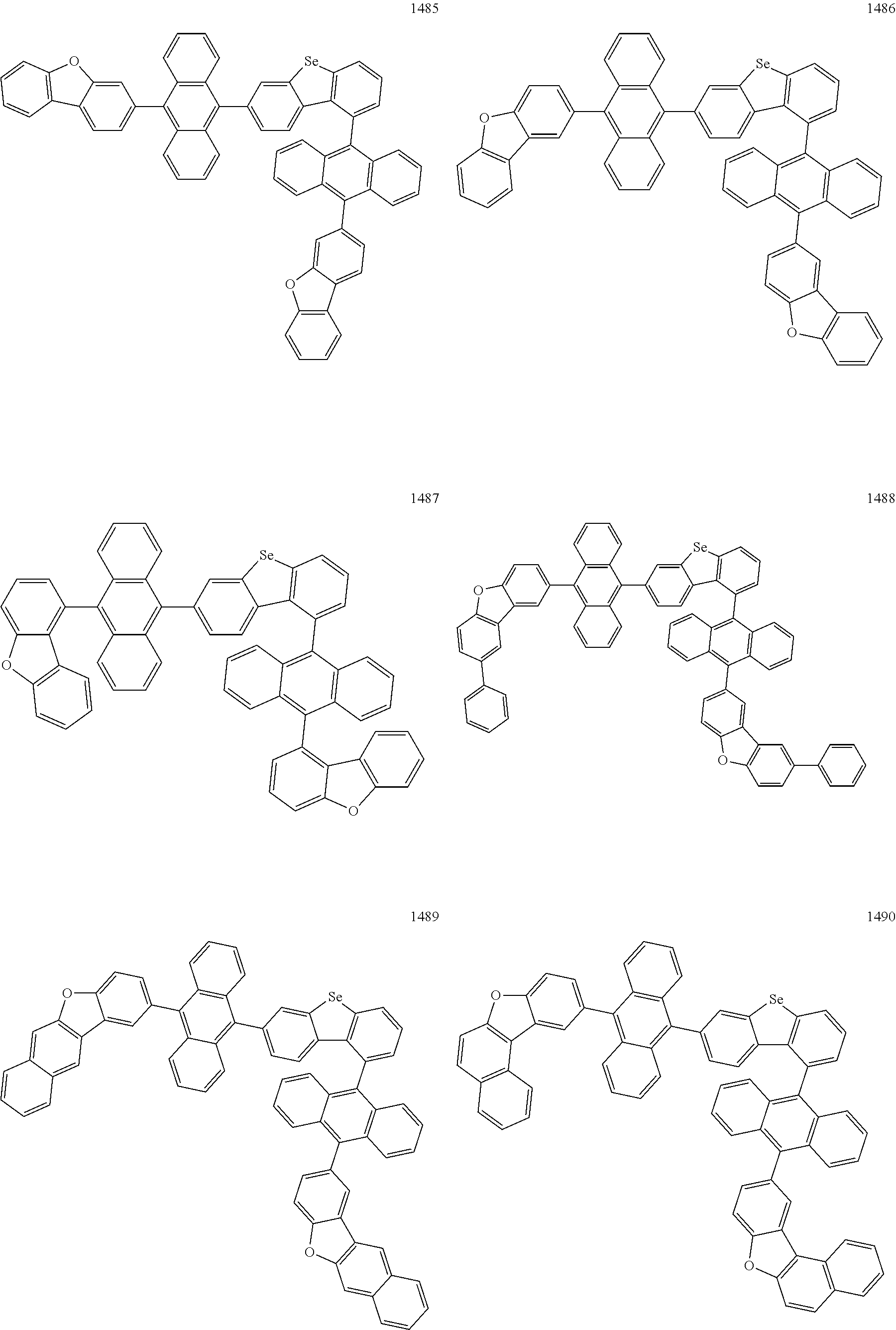

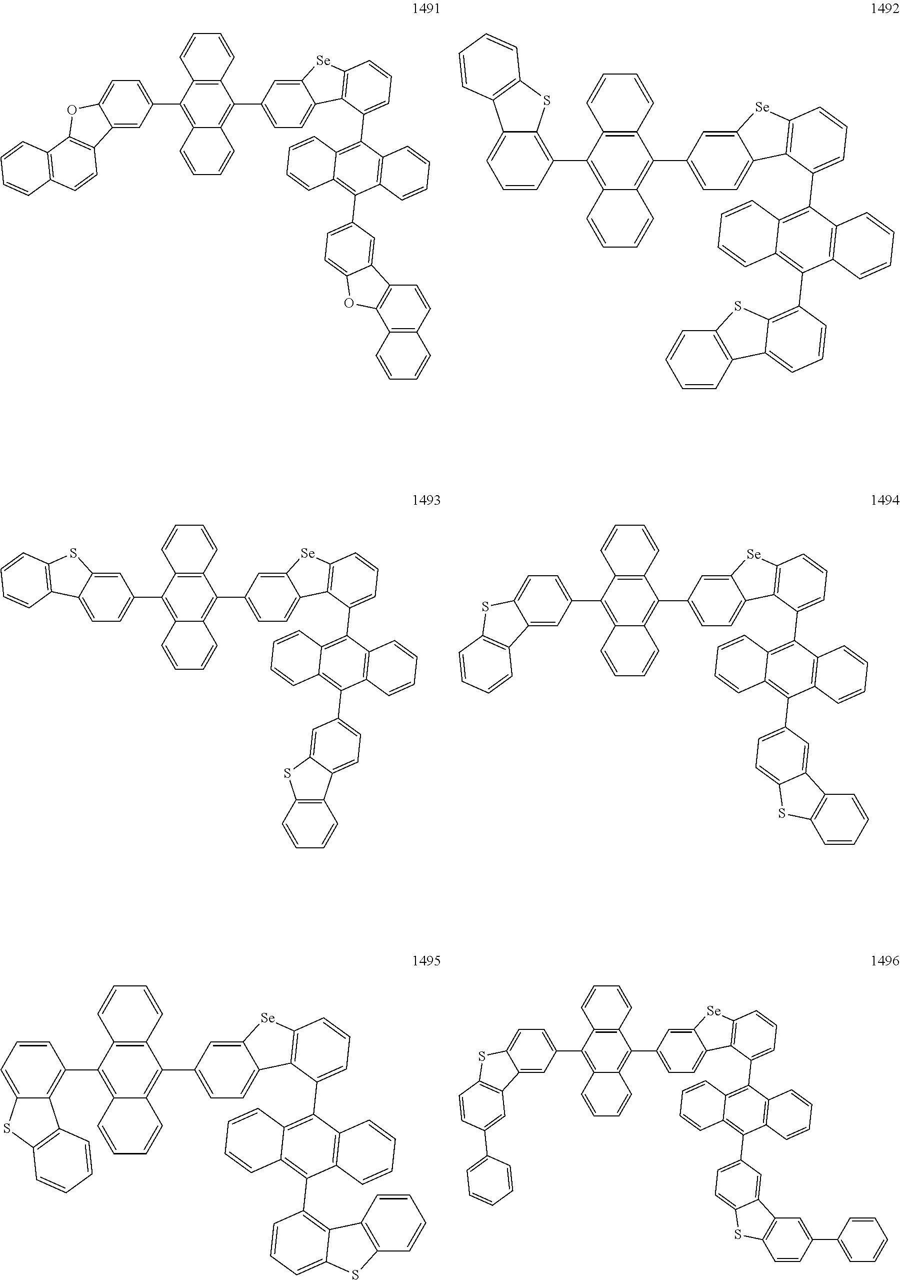

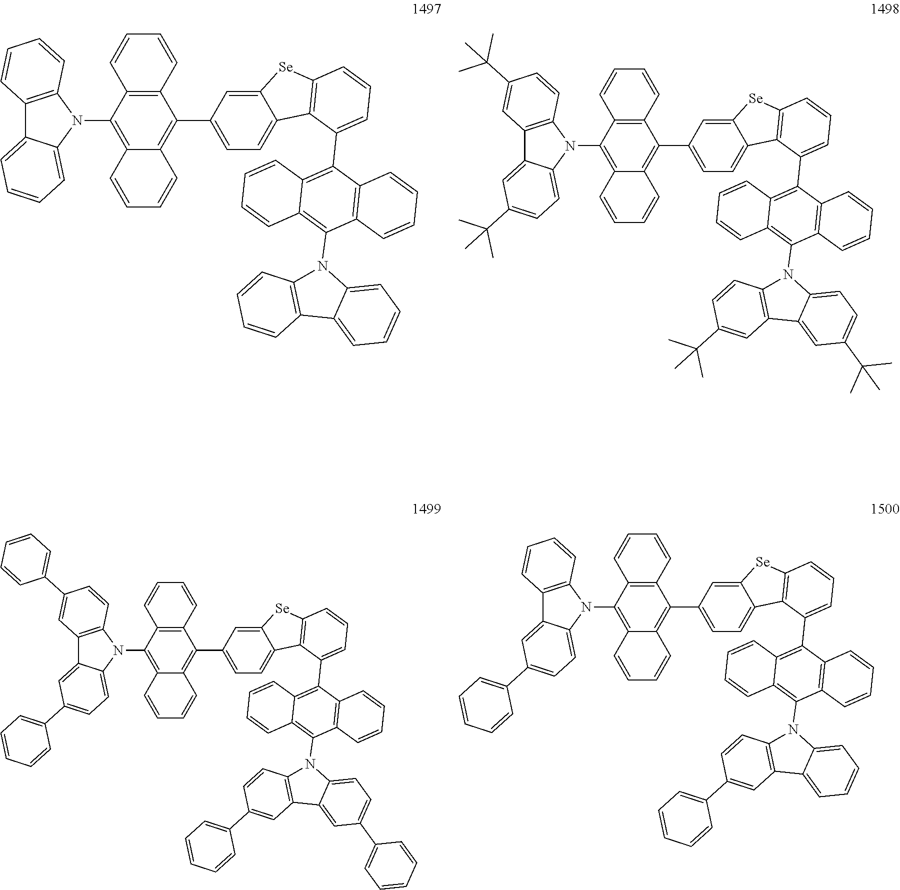

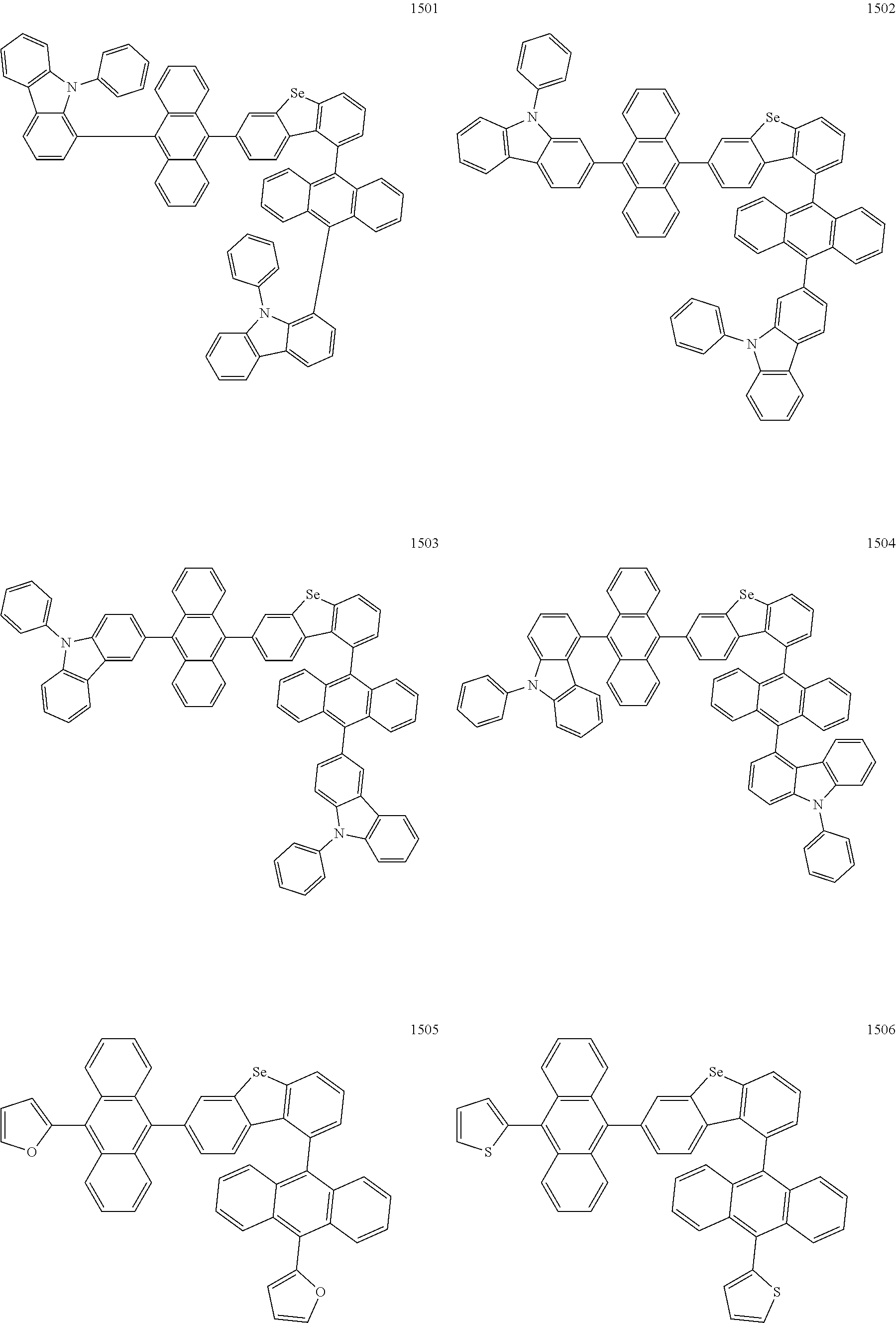

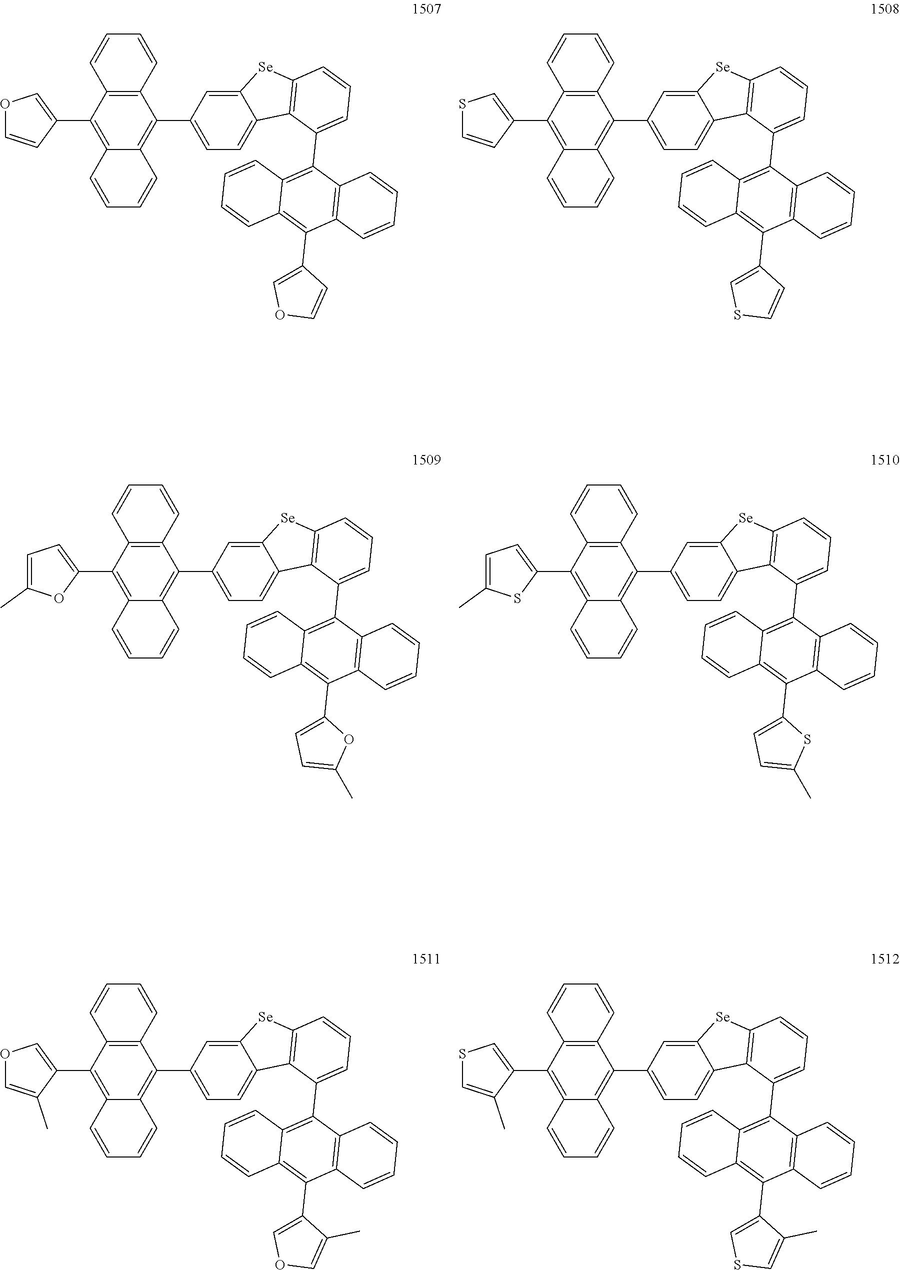

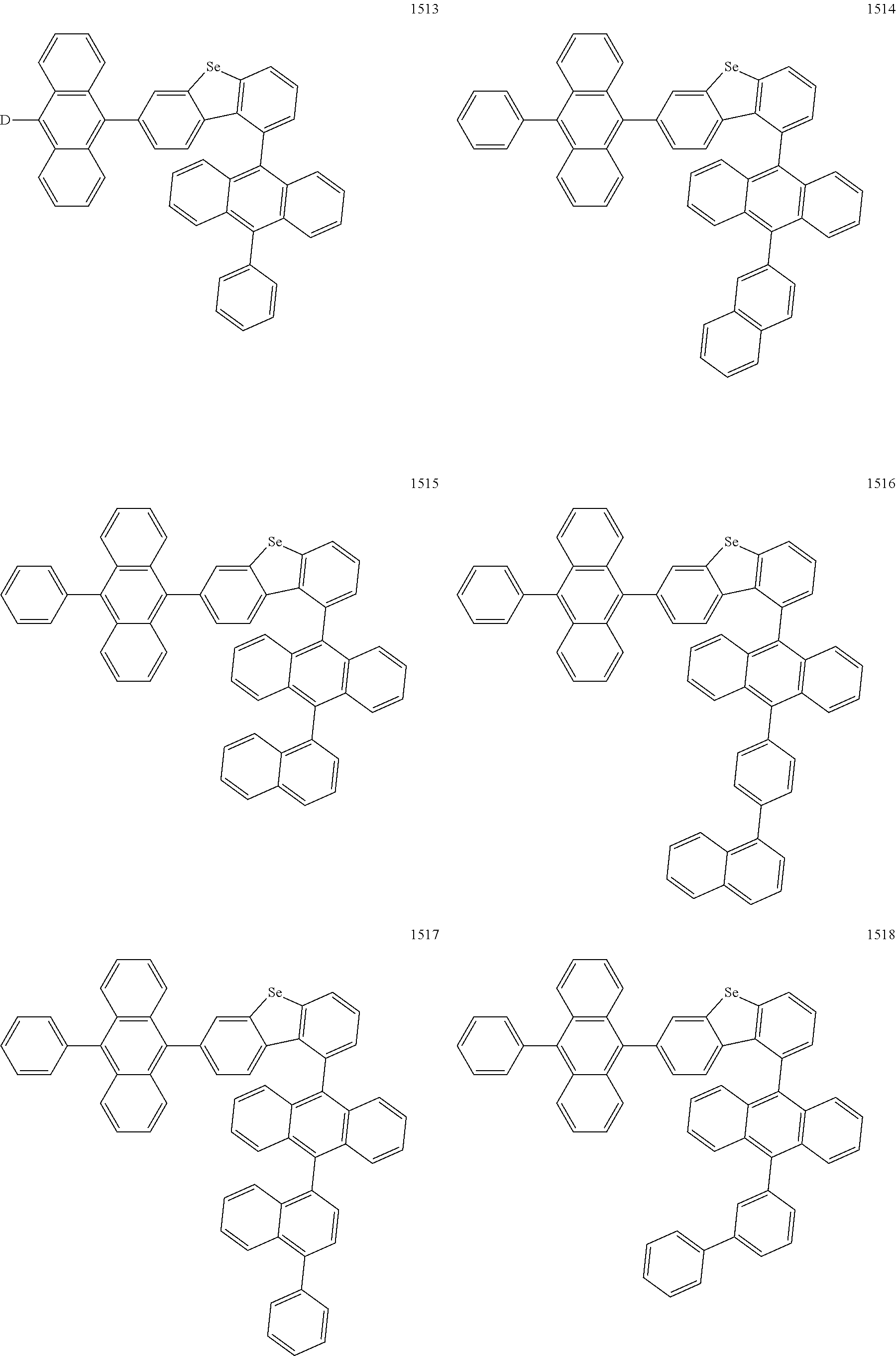

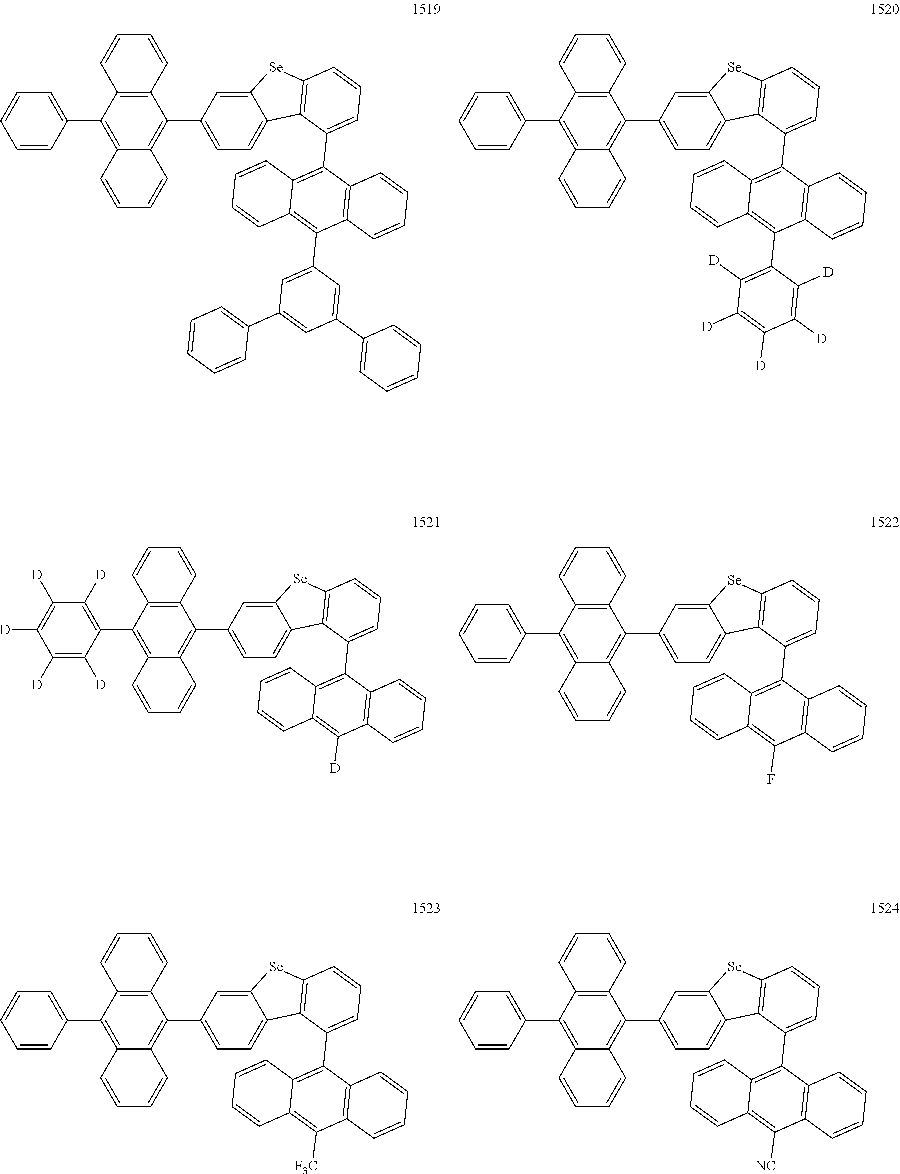

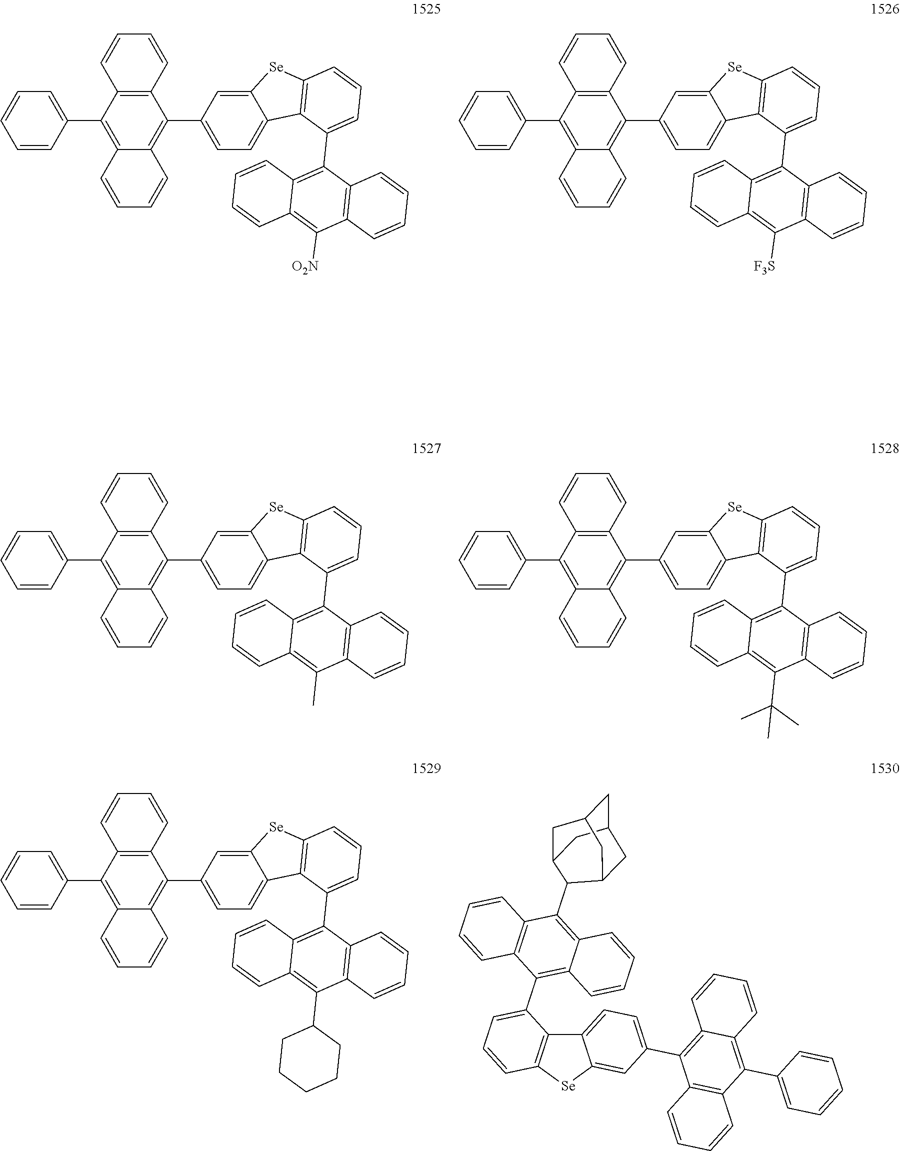

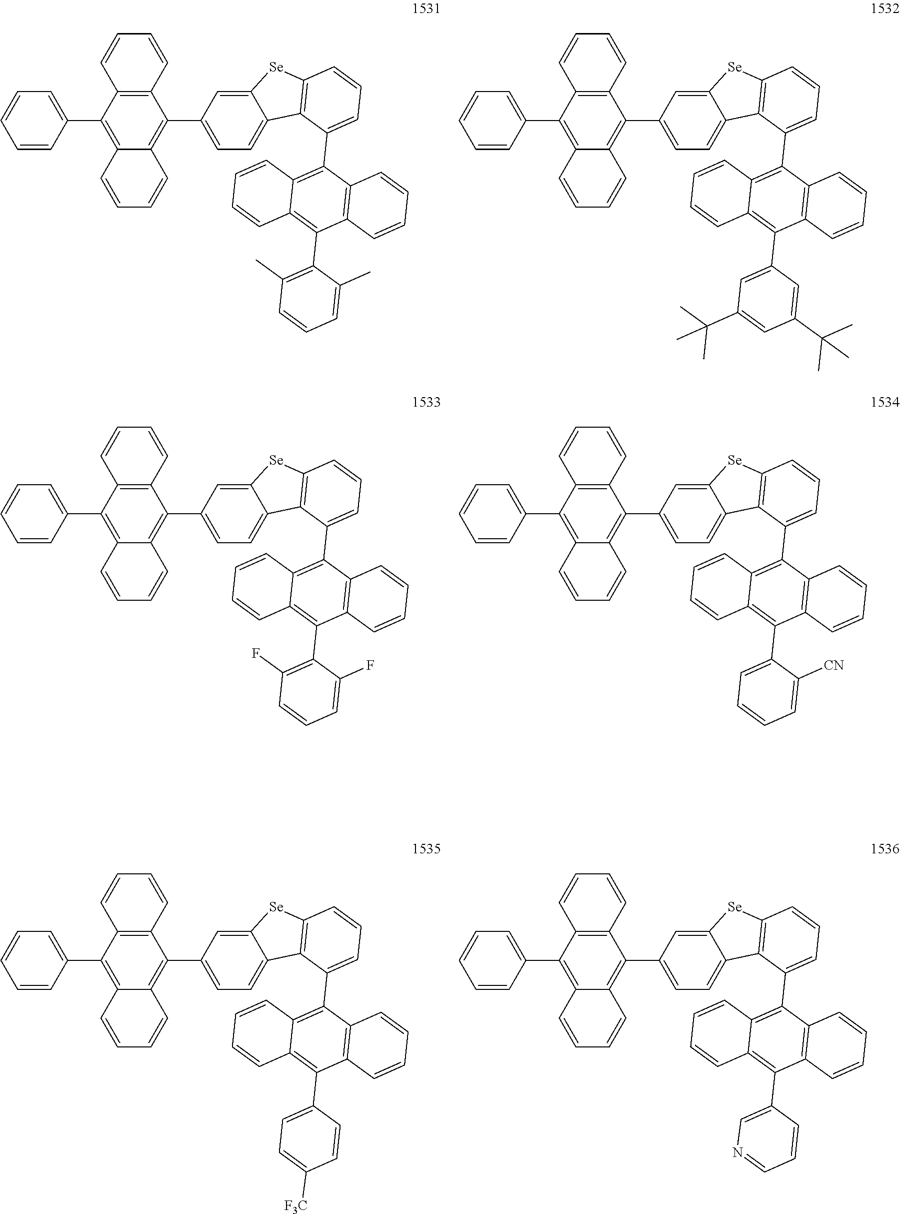

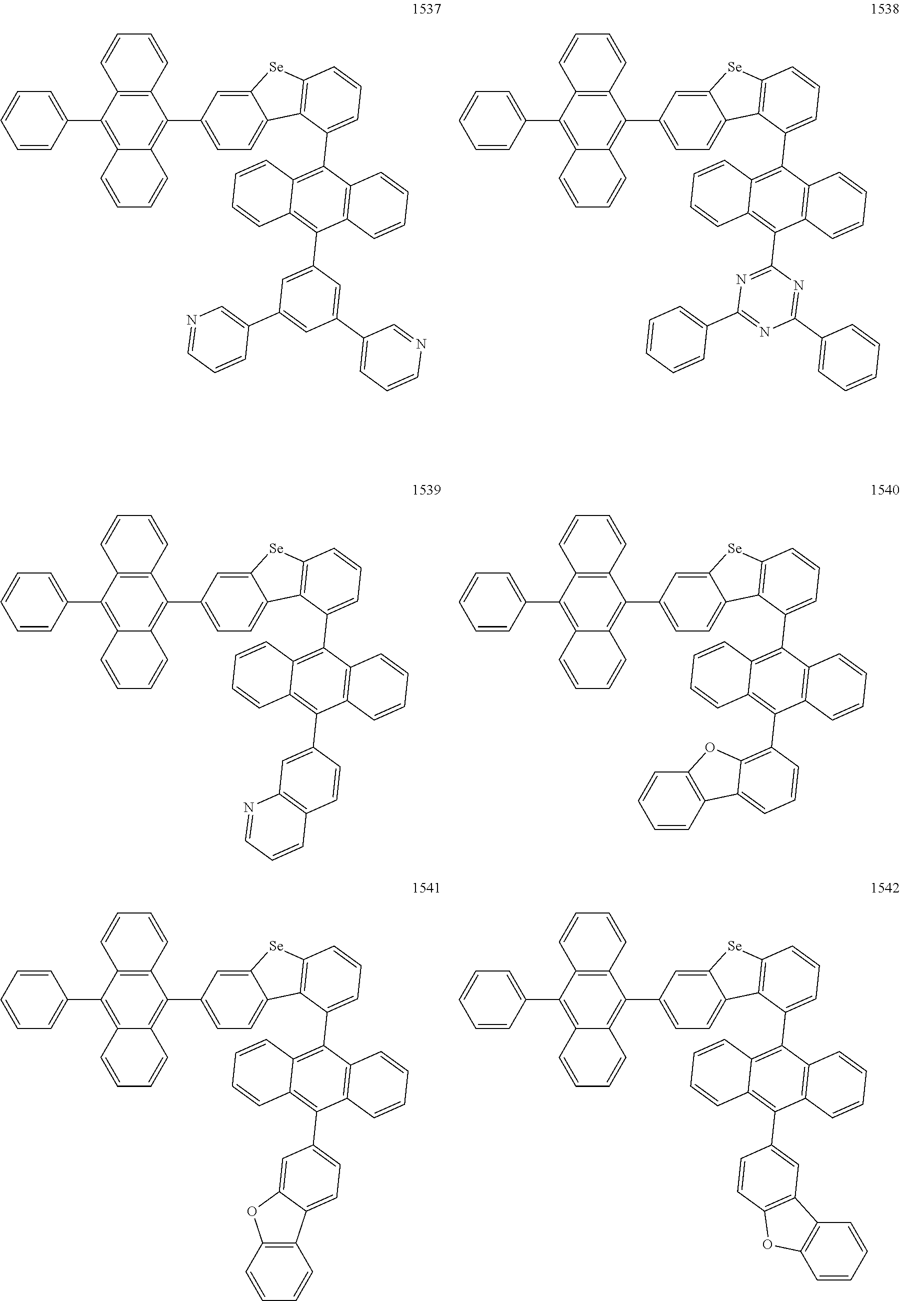

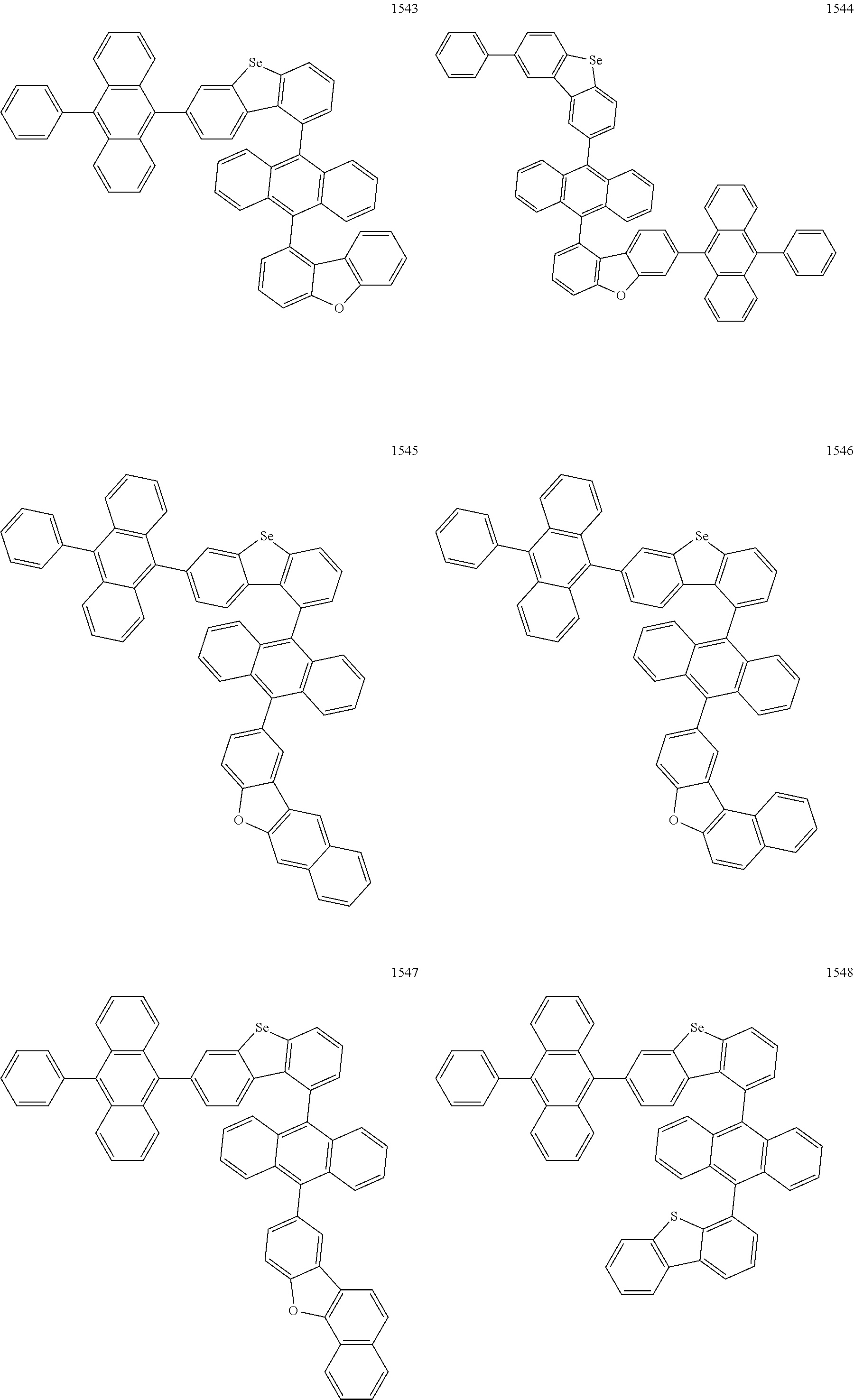

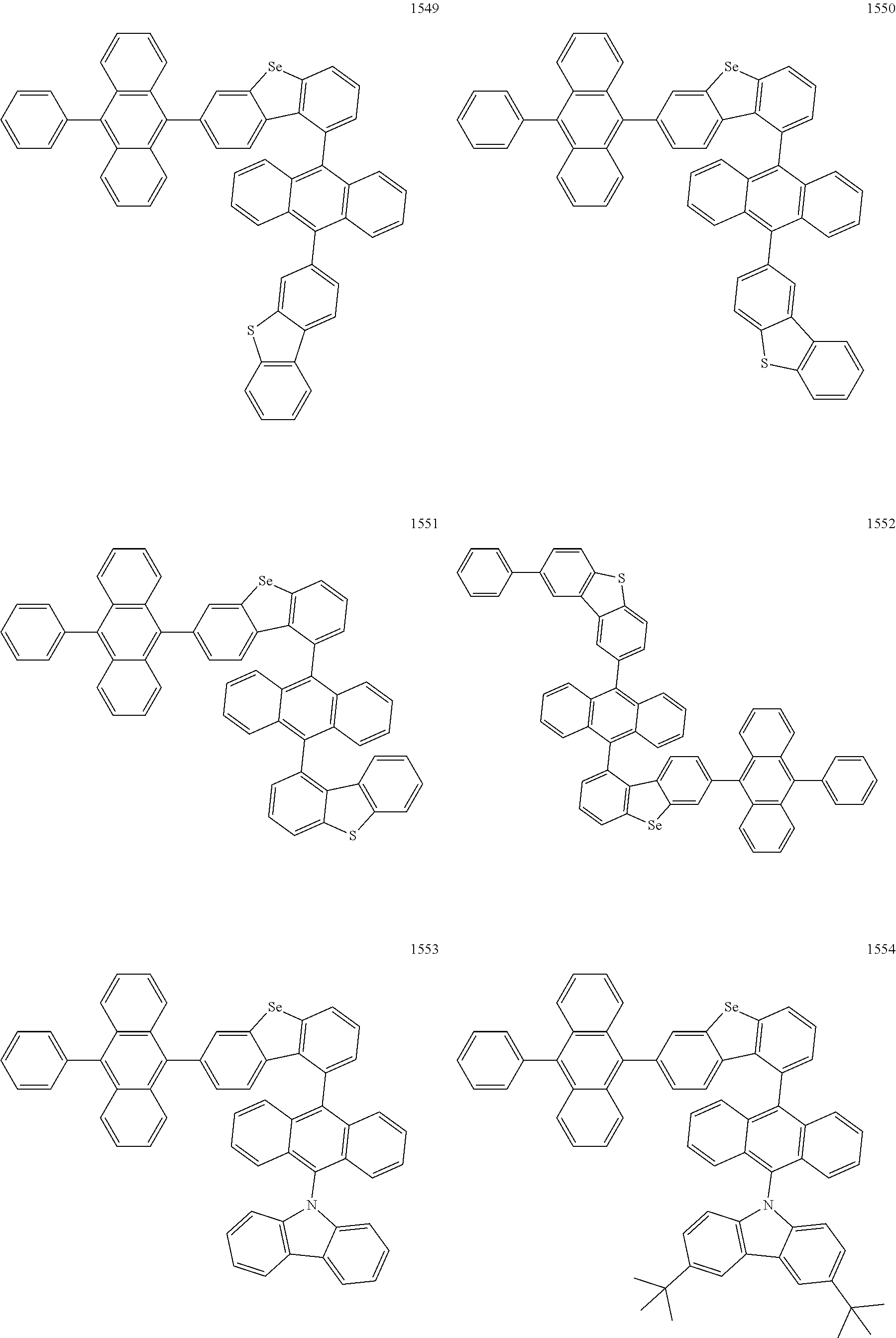

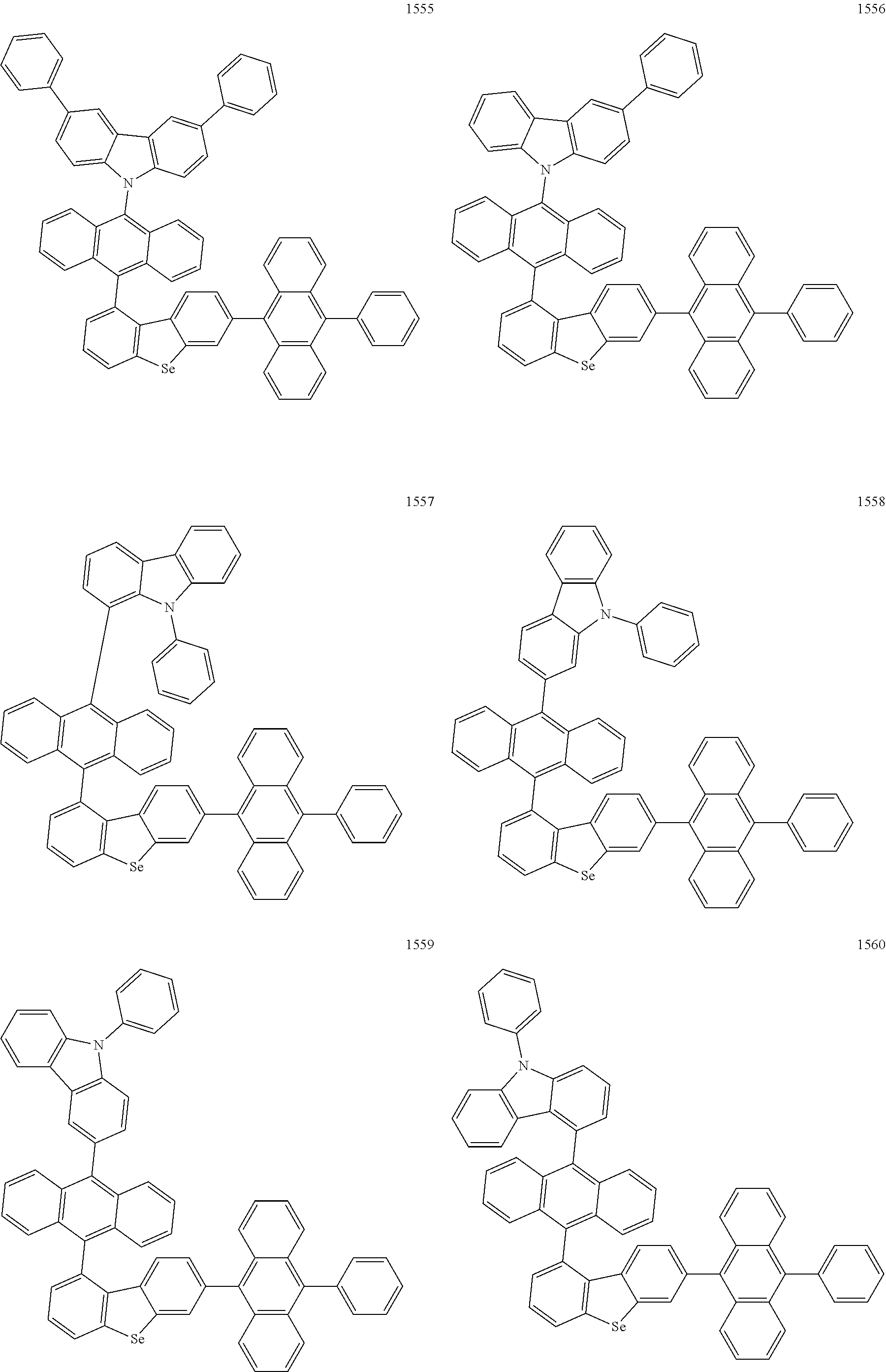

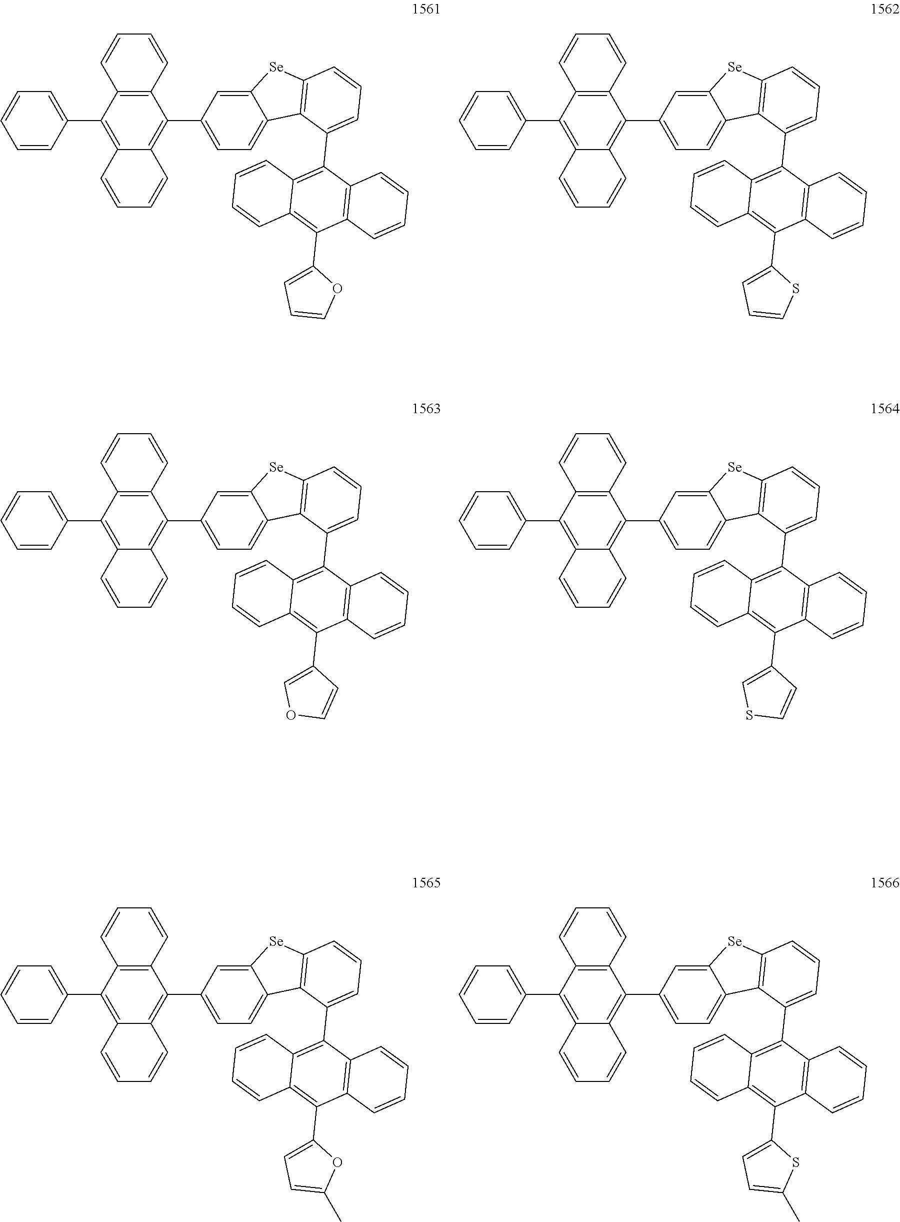

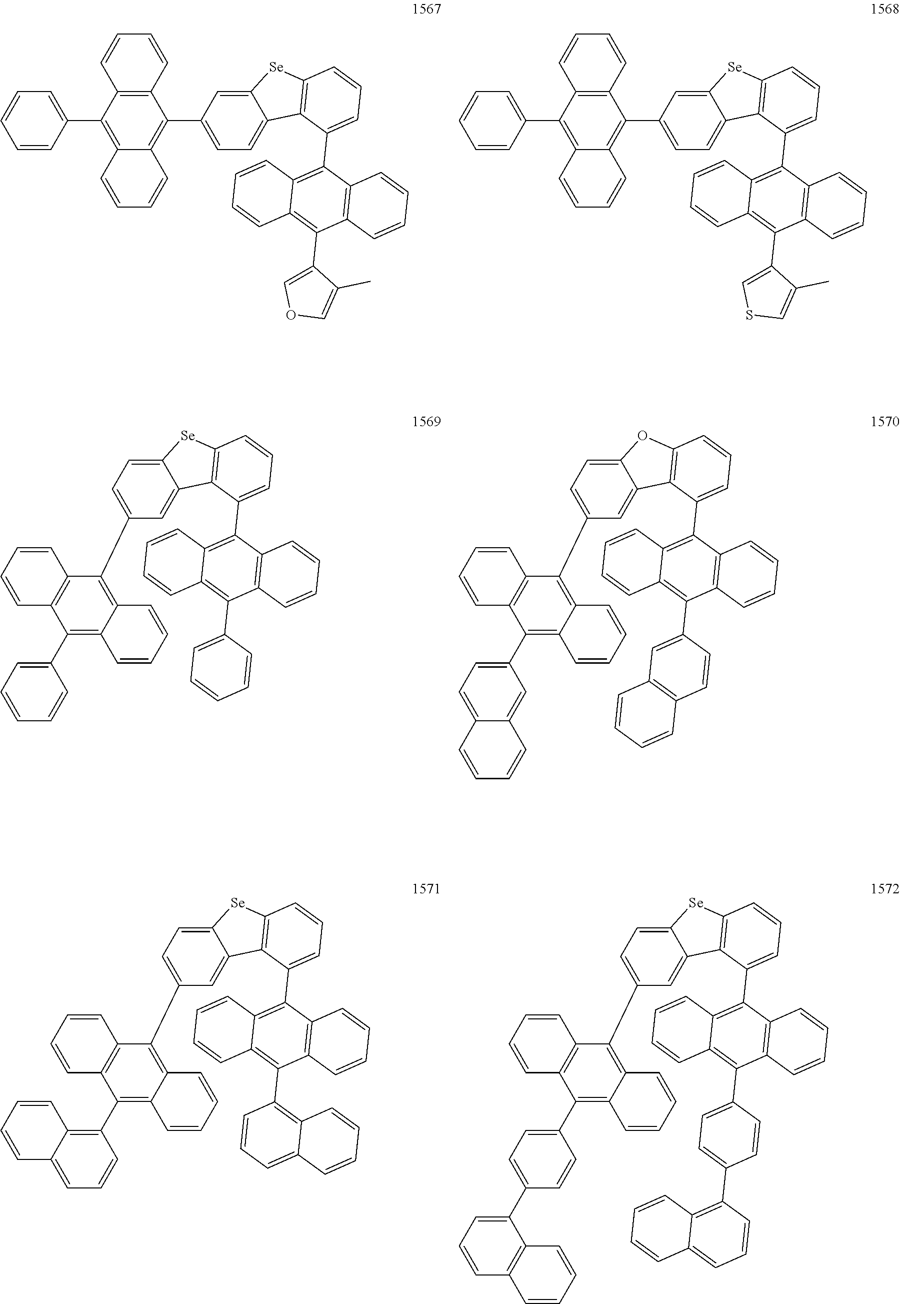

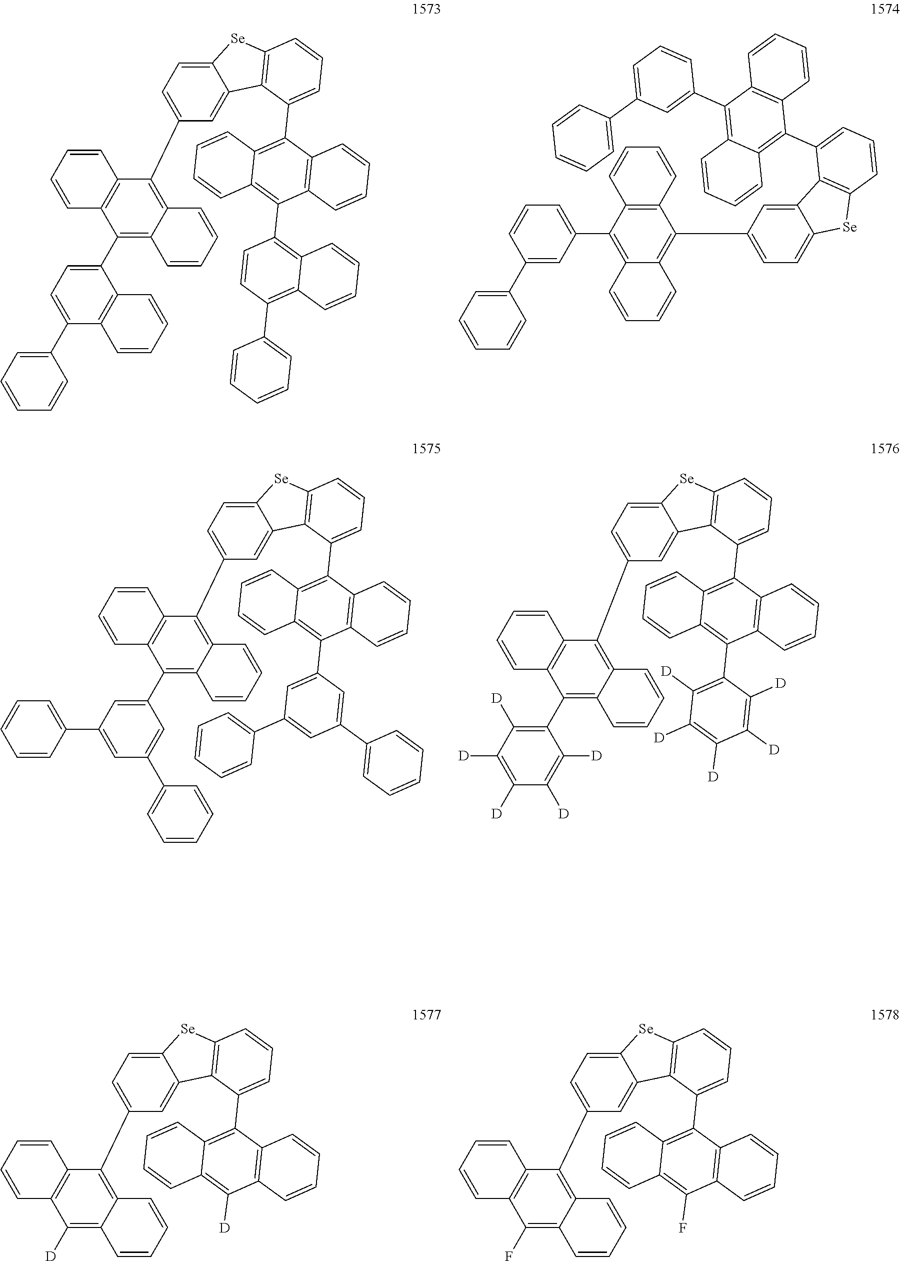

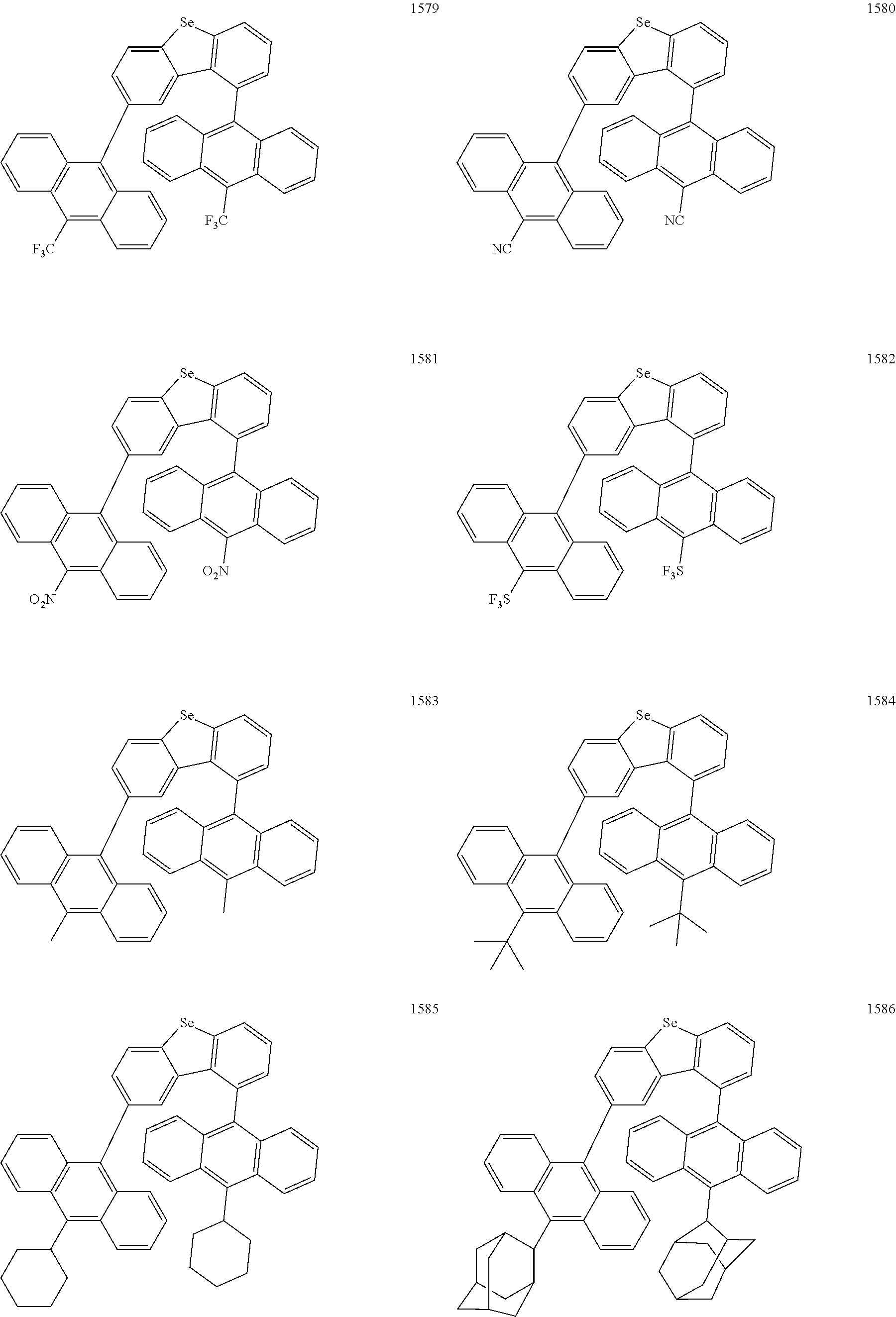

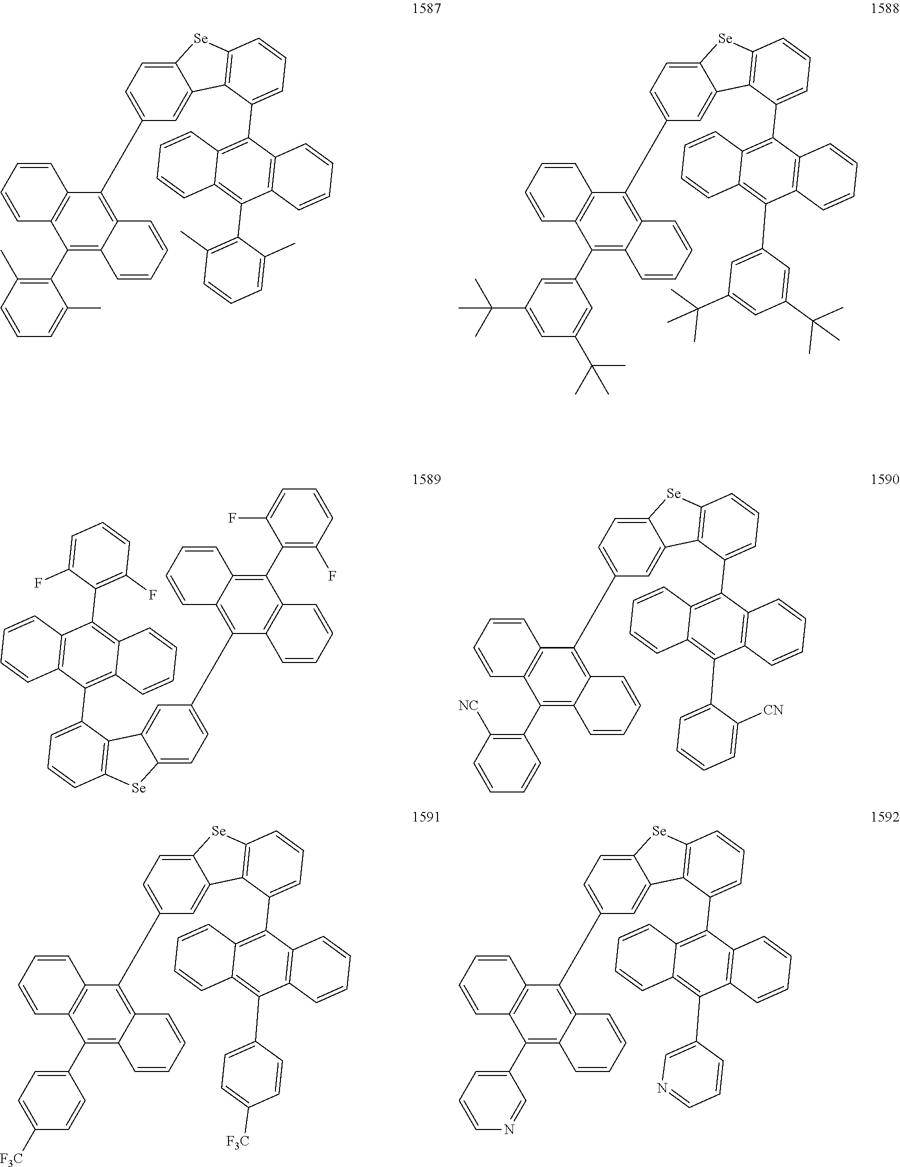

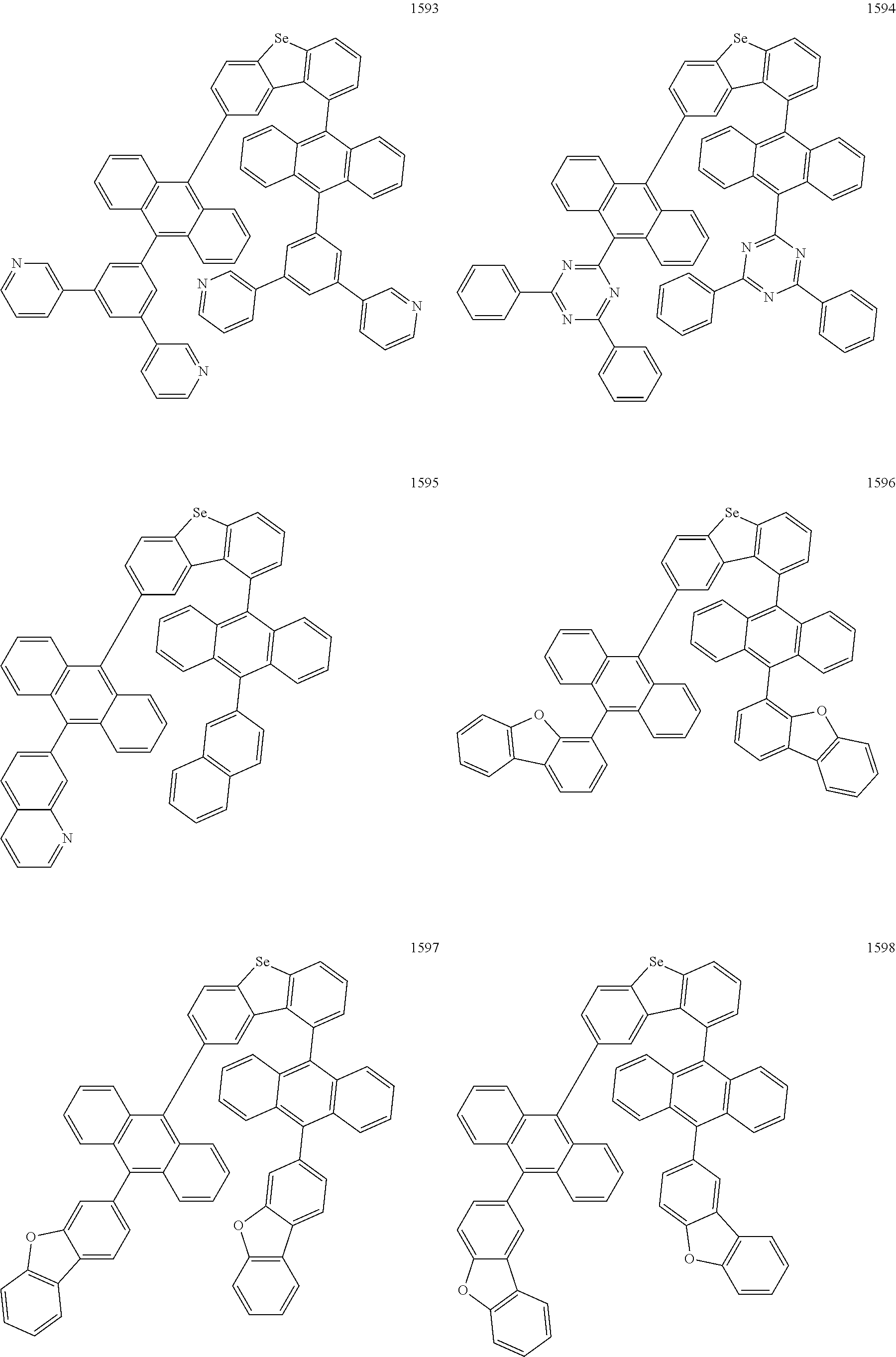

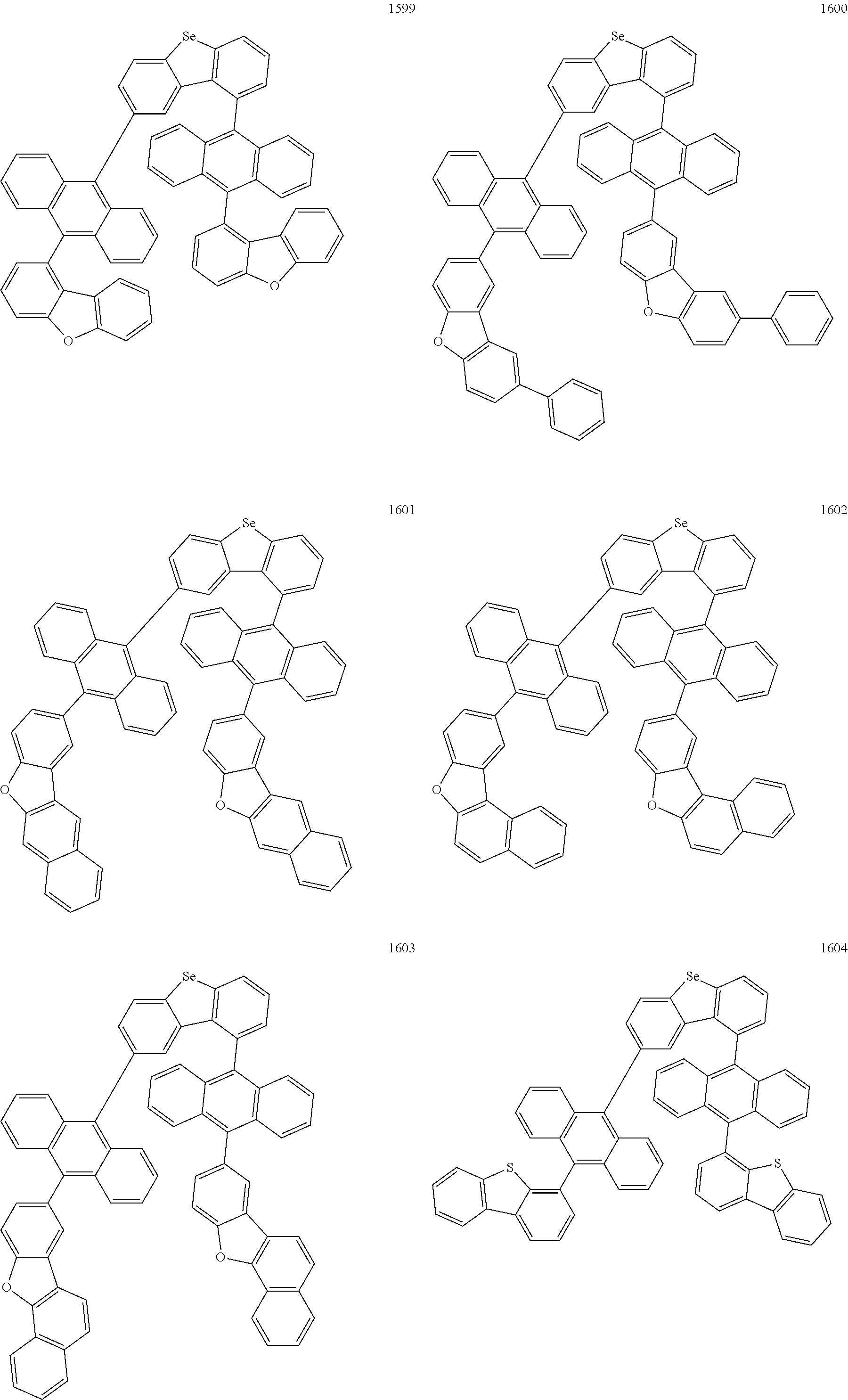

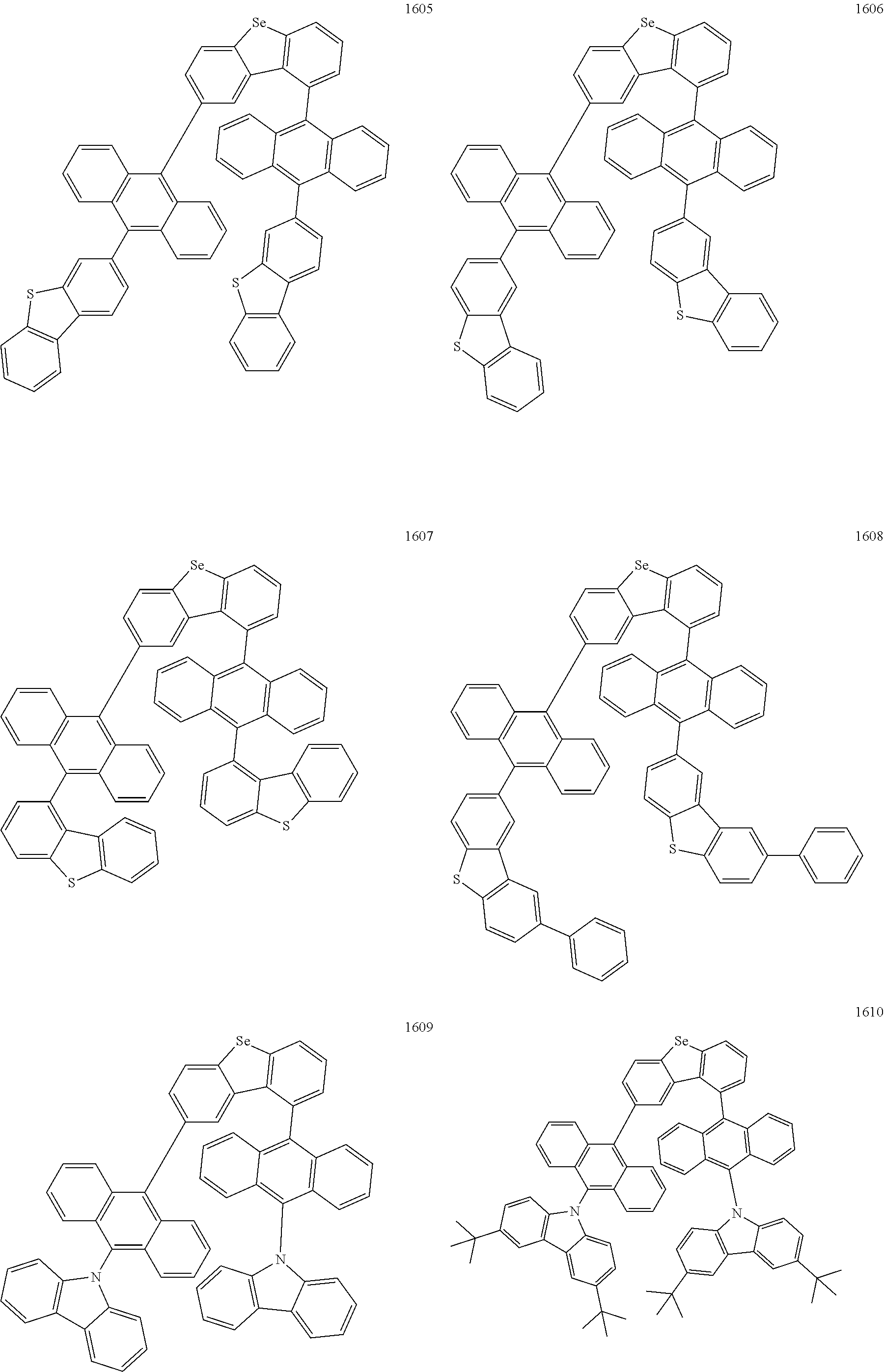

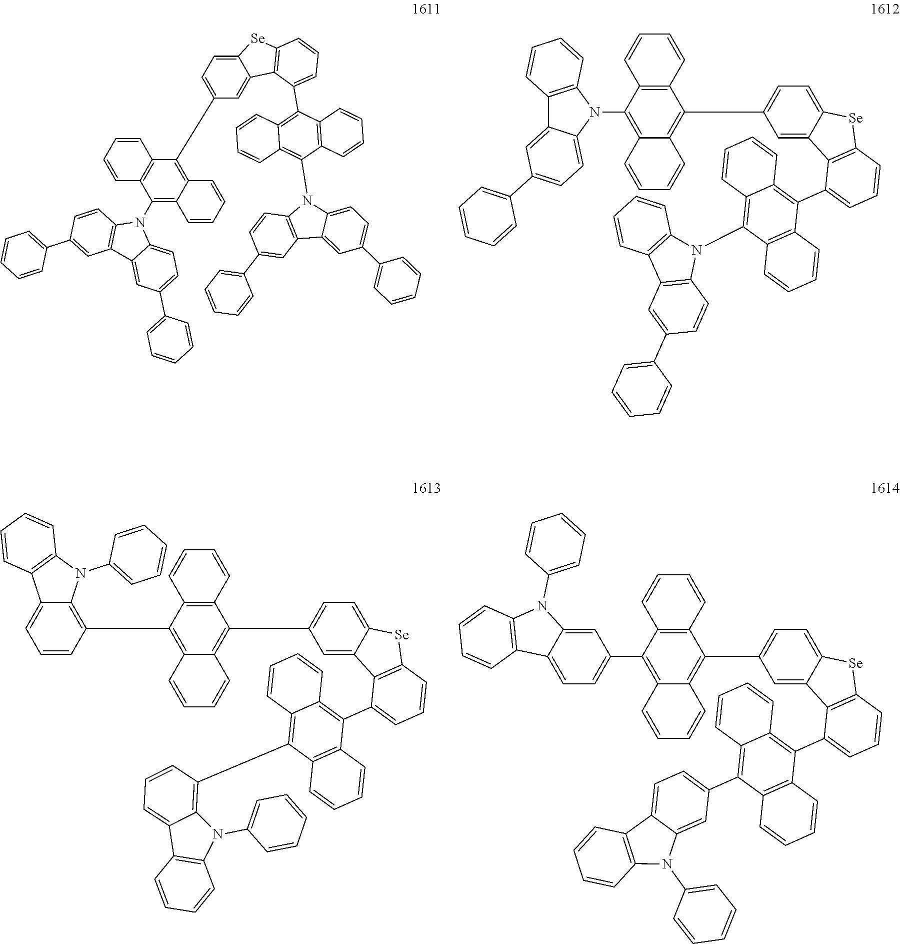

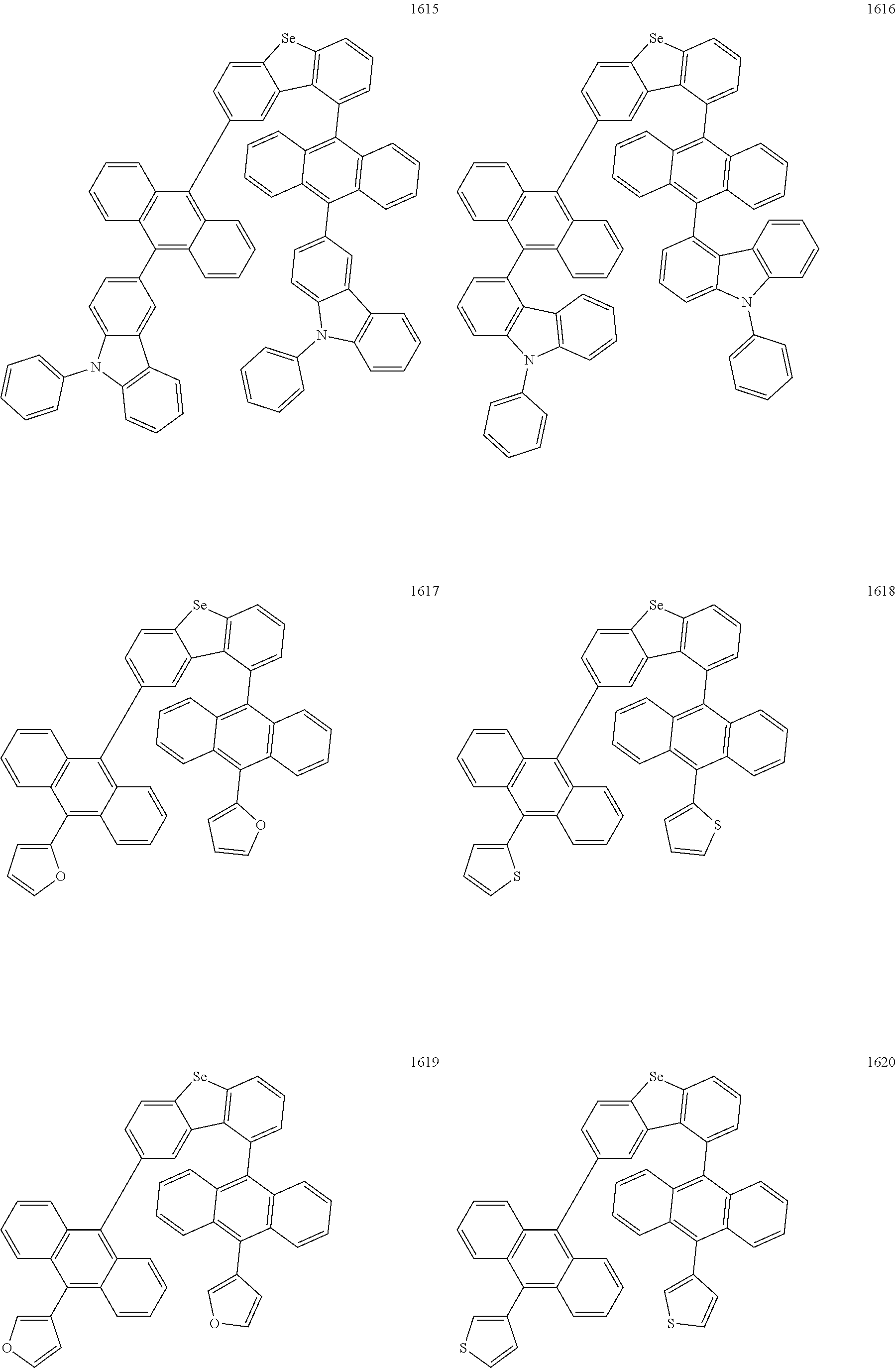

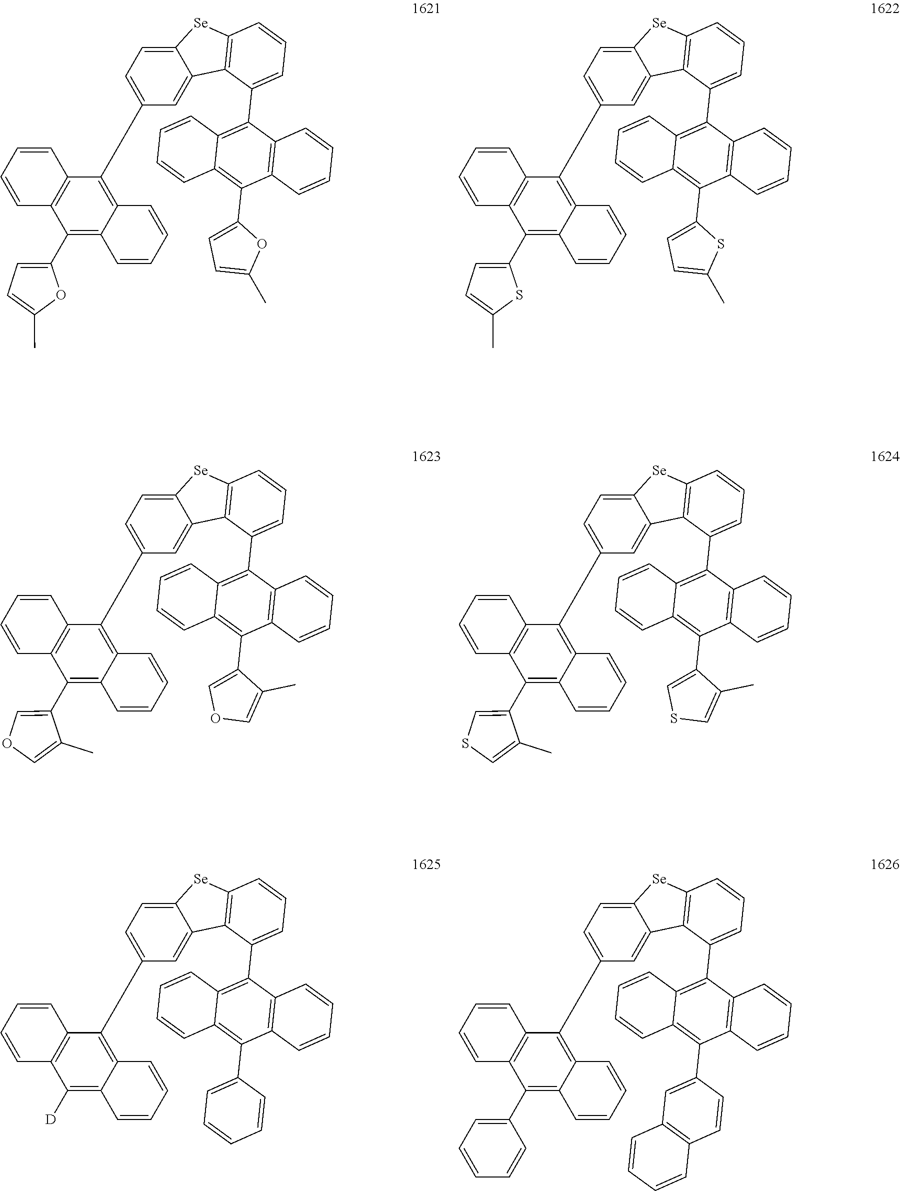

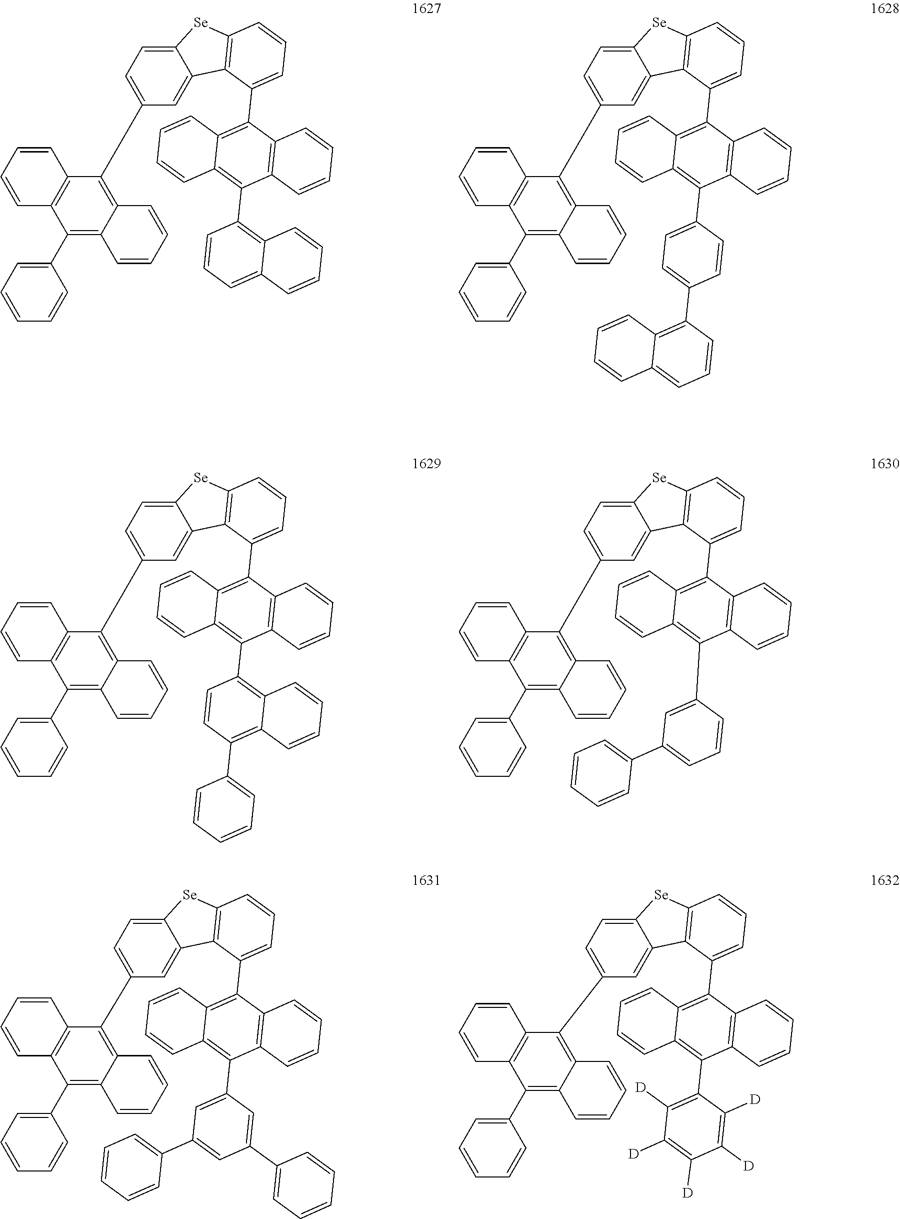

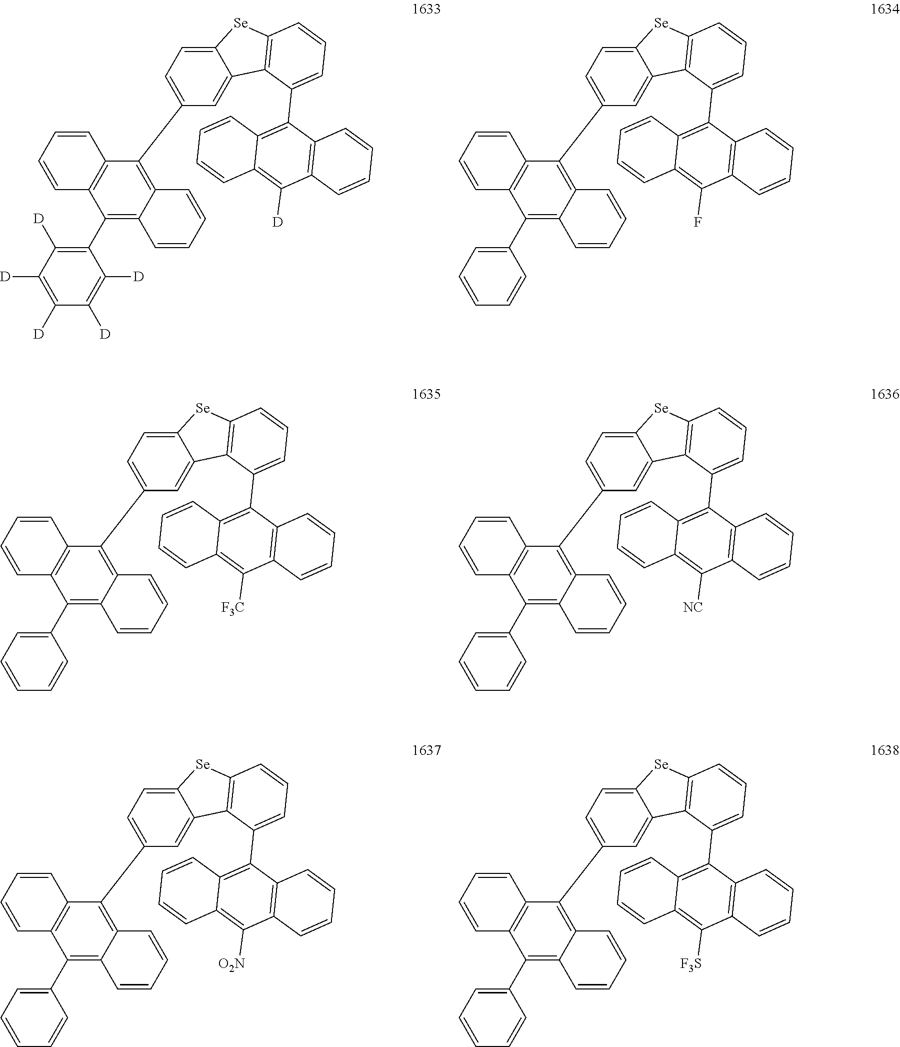

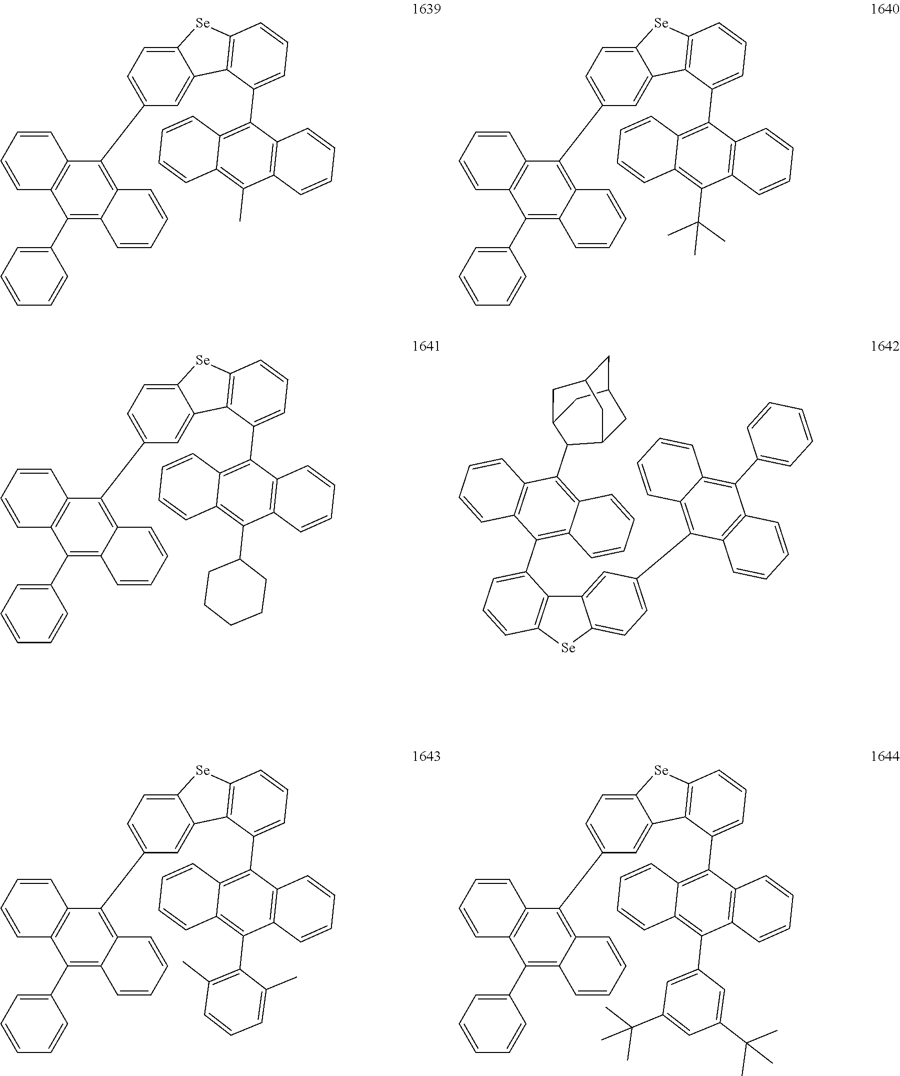

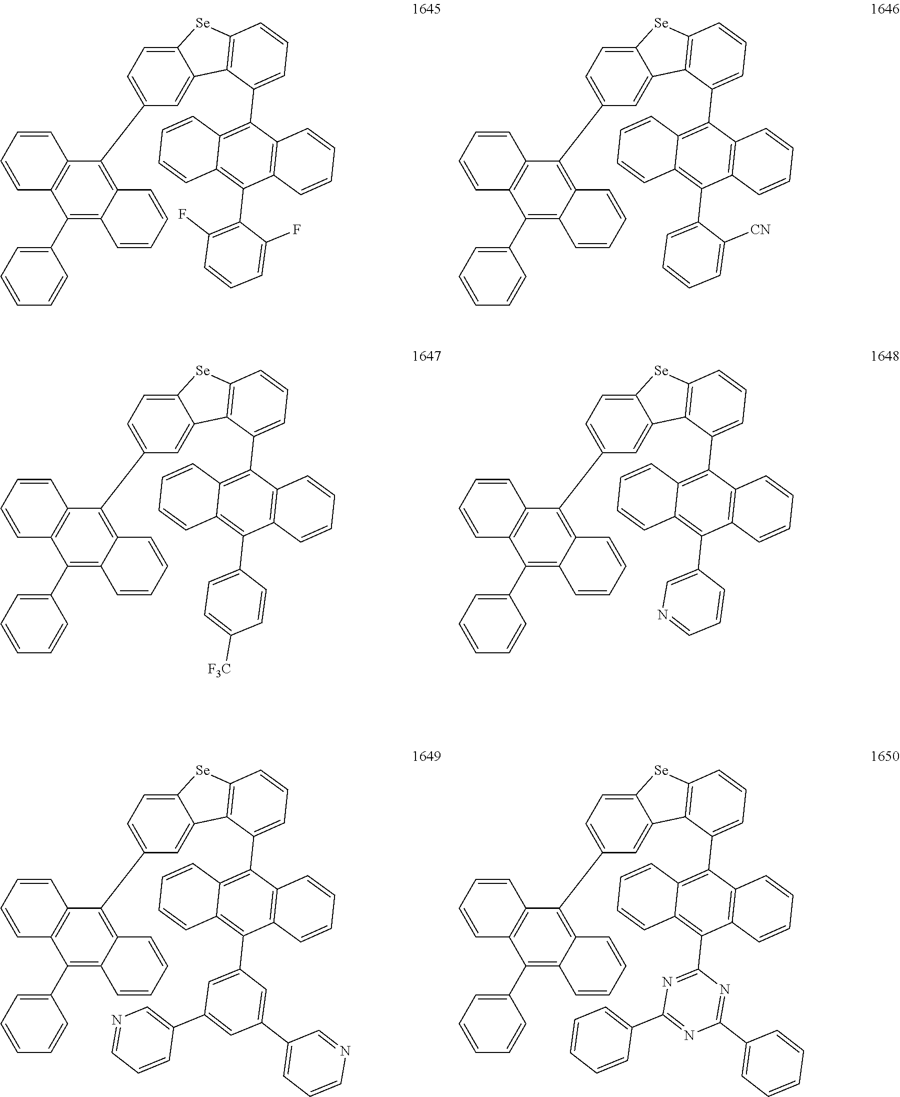

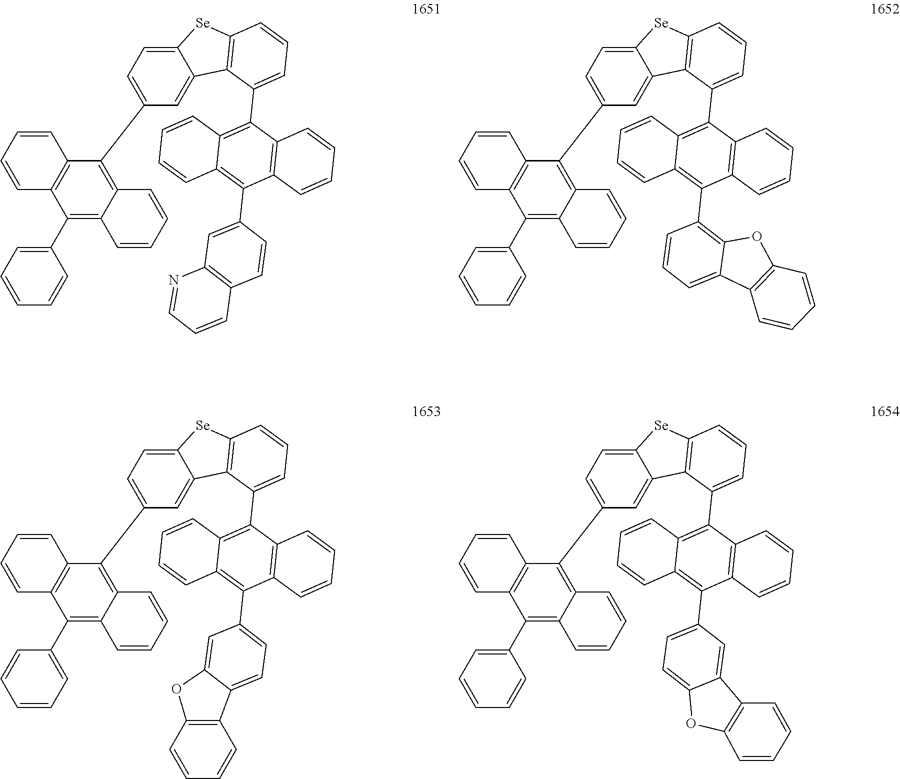 [SMILES: c1ccc(-c2c3ccccc3c(-c3ccc4[se]c5cccc(-c6c7ccccc7c(-c7ccc8cccnc8c7)c7ccccc67)c5c4c3)c3ccccc23)cc1 ; c1ccc(-c2c3ccccc3c(-c3ccc4[se]c5cccc(-c6c7ccccc7c(-c7cccc8c7oc7ccccc78)c7ccccc67)c5c4c3)c3ccccc23)cc1 ; c1ccc(-c2c3ccccc3c(-c3ccc4[se]c5cccc(-c6c7ccccc7c(-c7ccc8c(c7)oc7ccccc78)c7ccccc67)c5c4c3)c3ccccc23)cc1 ; c1ccc(-c2c3ccccc3c(-c3ccc4[se]c5cccc(-c6c7ccccc7c(-c7ccc8oc9ccccc9c8c7)c7ccccc67)c5c4c3)c3ccccc23)cc1]

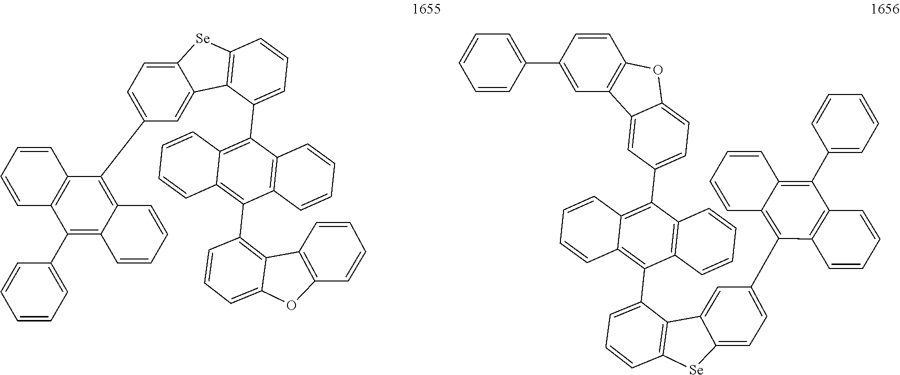 [SMILES: c1ccc(-c2c3ccccc3c(-c3ccc4[se]c5cccc(-c6c7ccccc7c(-c7cccc8oc9ccccc9c78)c7ccccc67)c5c4c3)c3ccccc23)cc1 ; c1ccc(-c2ccc3oc4ccc(-c5c6ccccc6c(-c6cccc7[se]c8ccc(-c9c%10ccccc%10c(-c%10ccccc%10)c%10ccccc9%10)cc8c67)c6ccccc56)cc4c3c2)cc1]

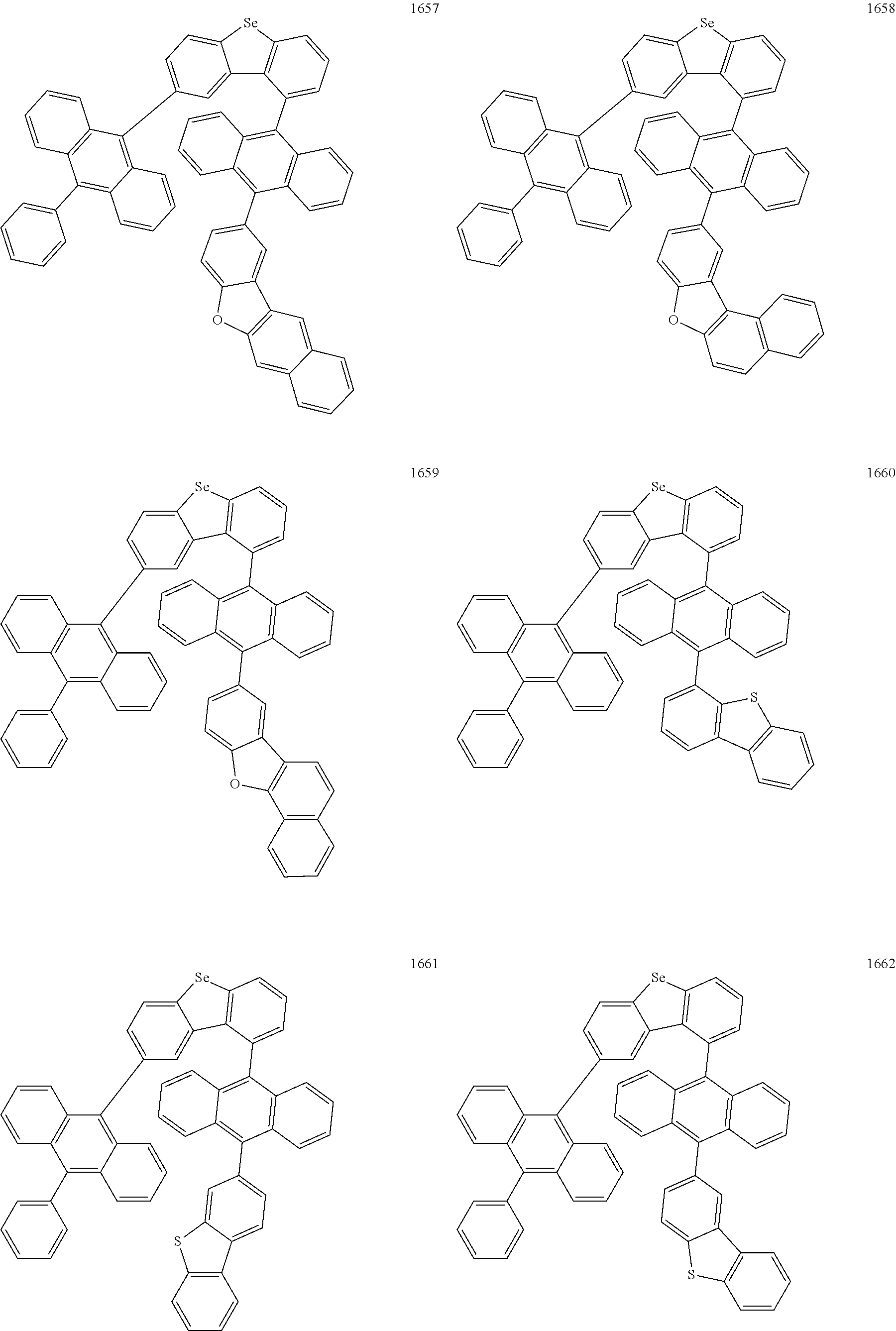

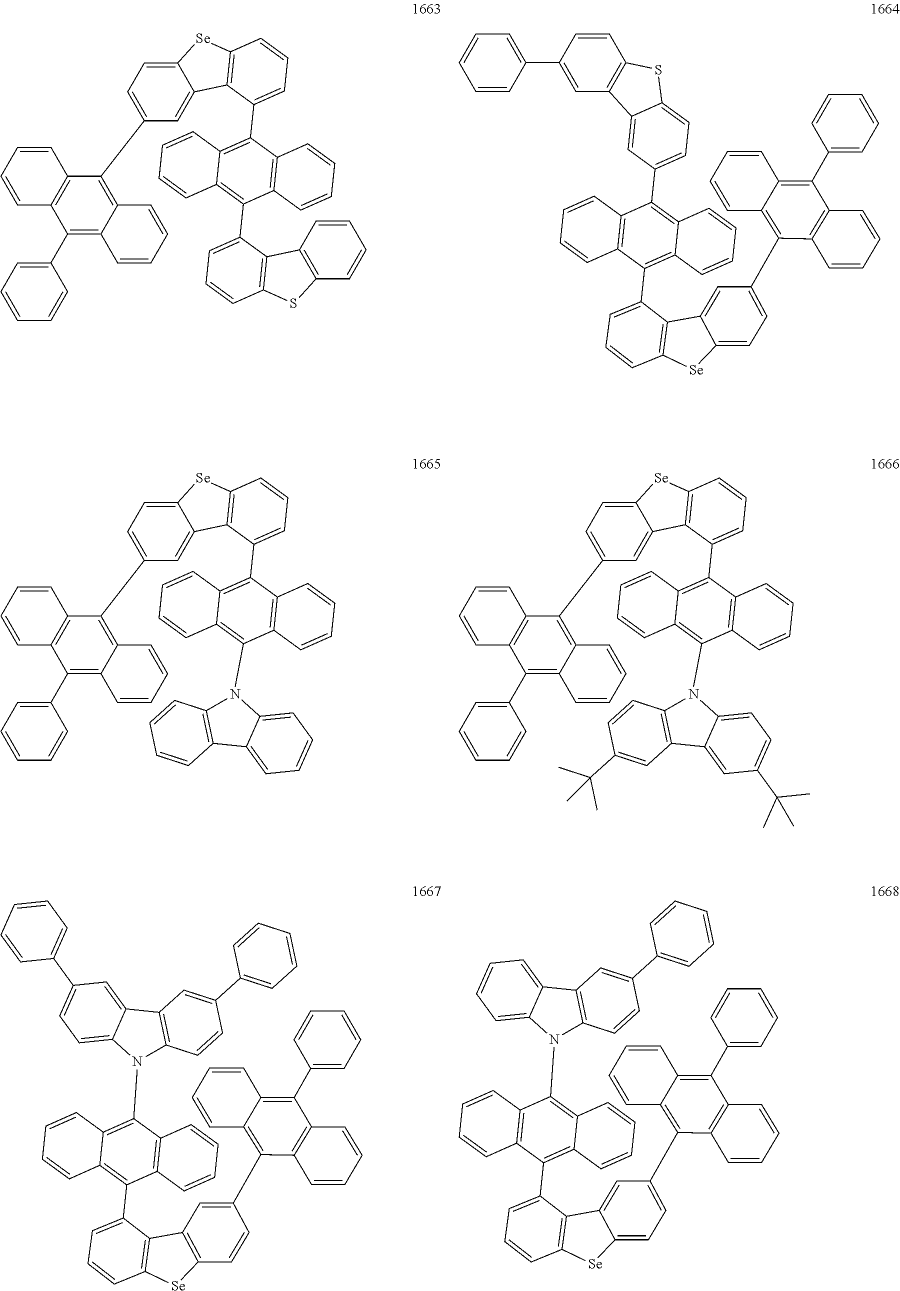

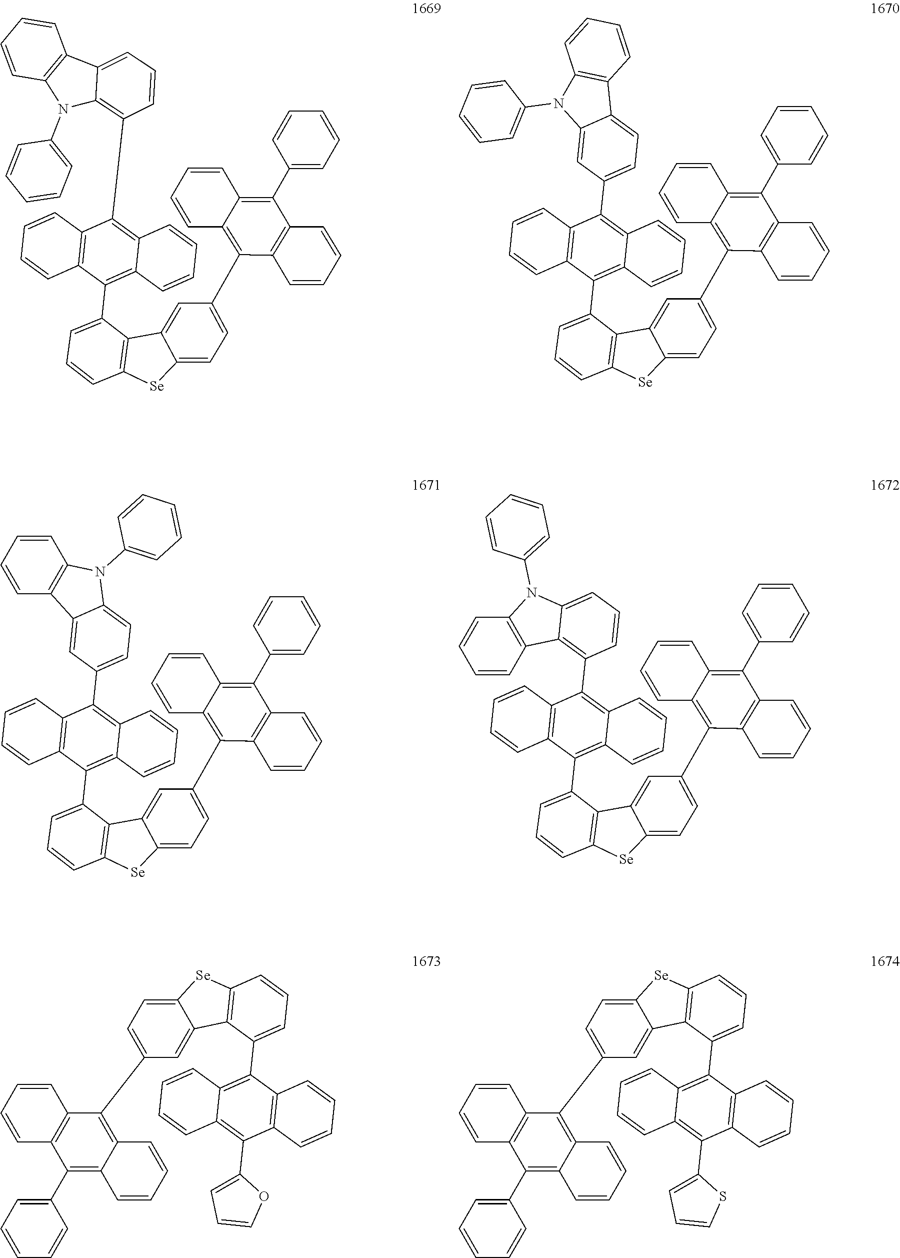

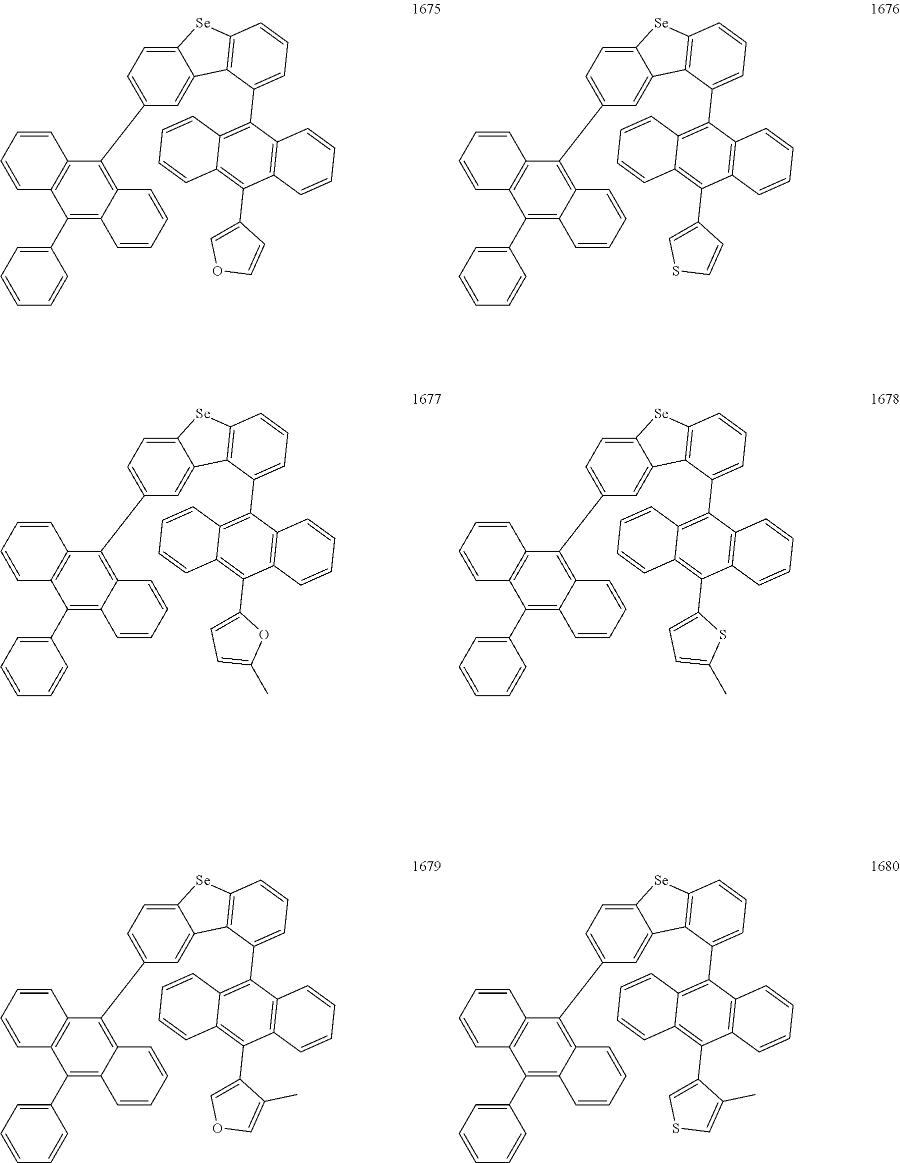

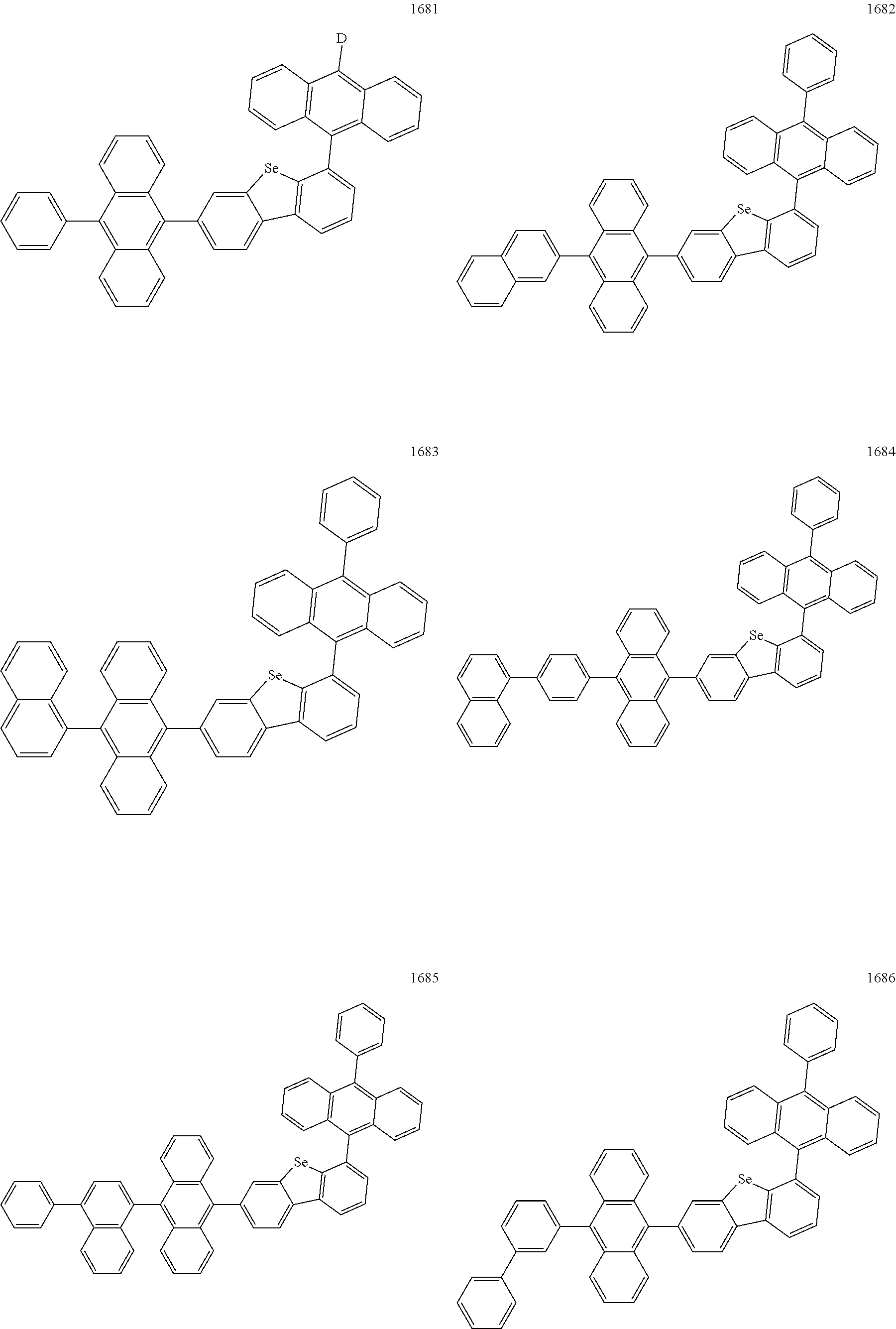

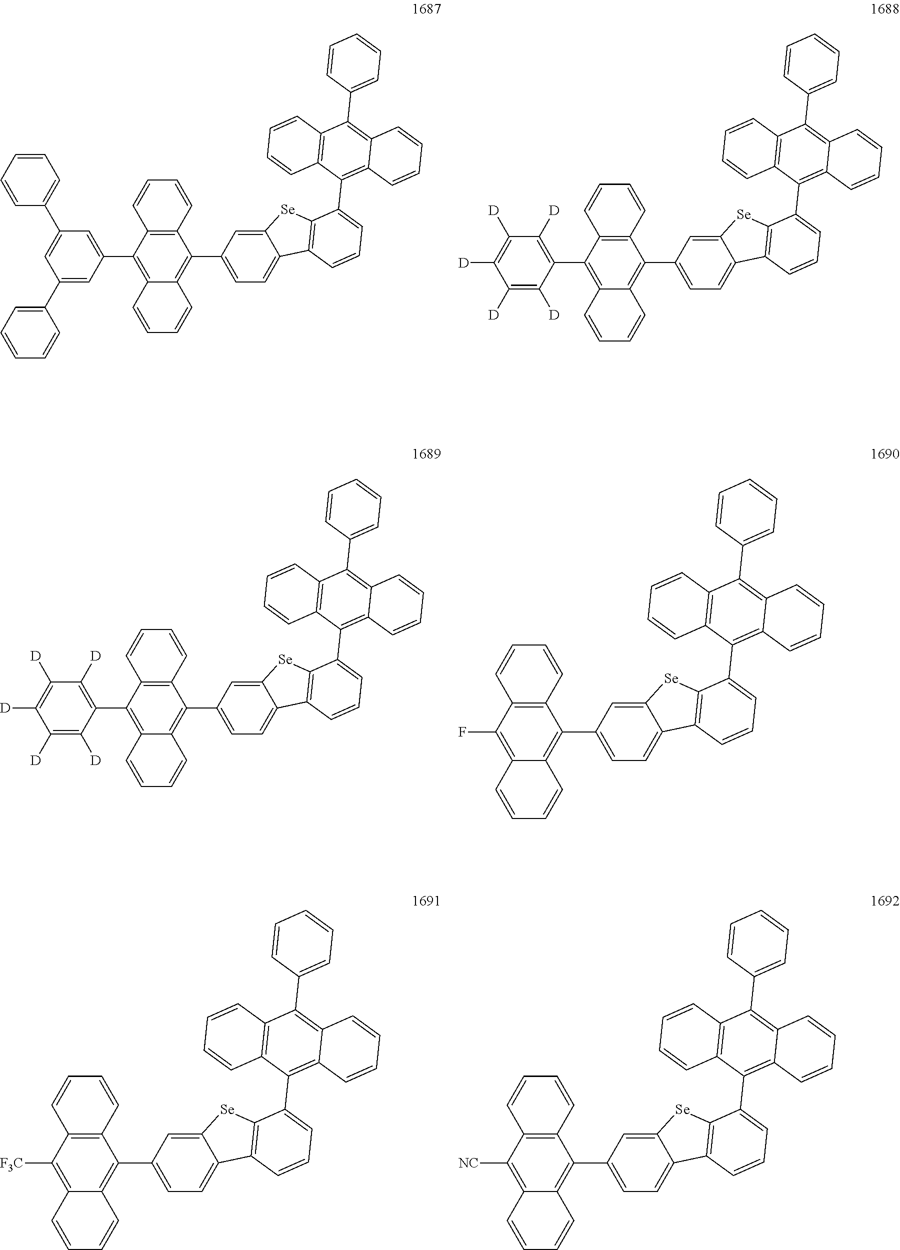

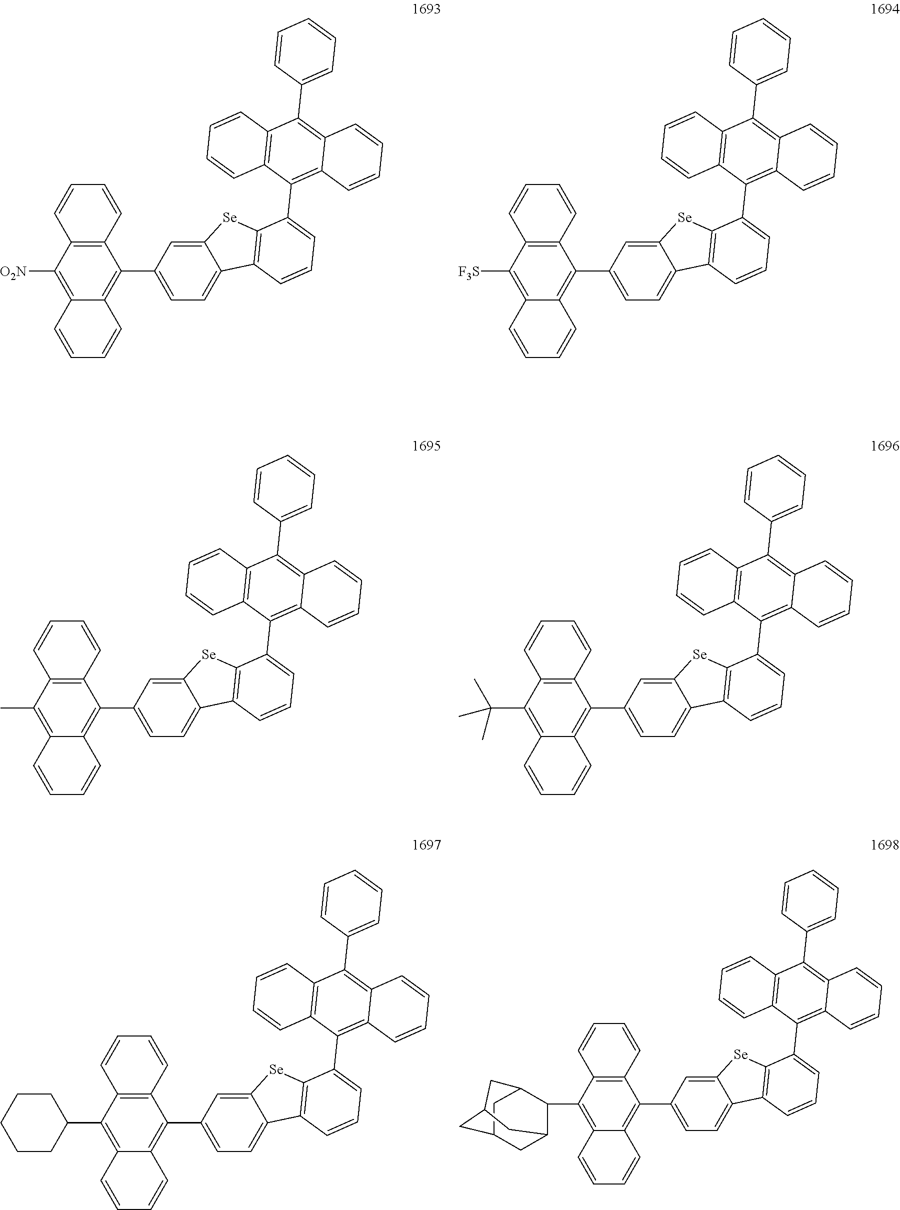

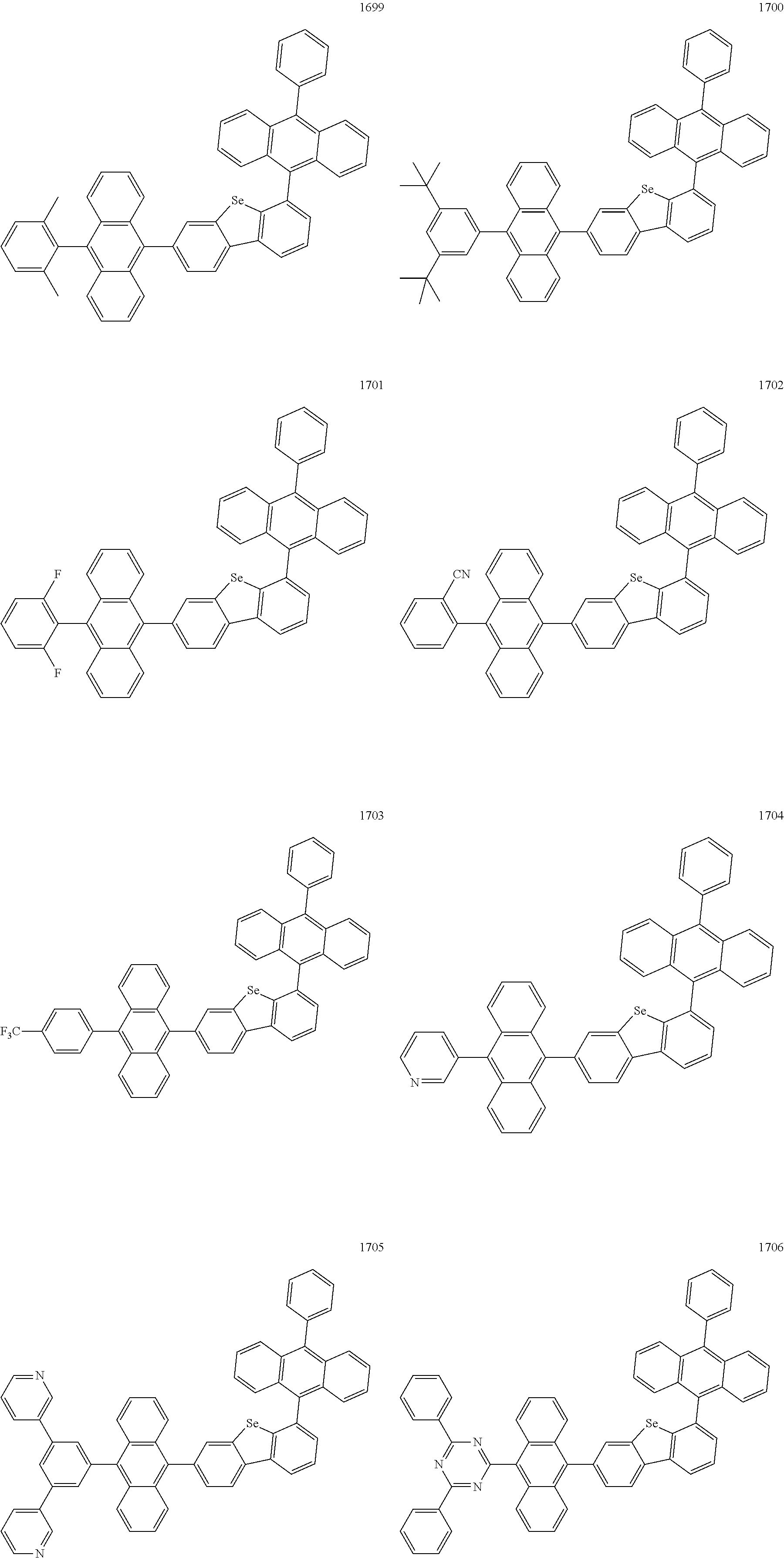

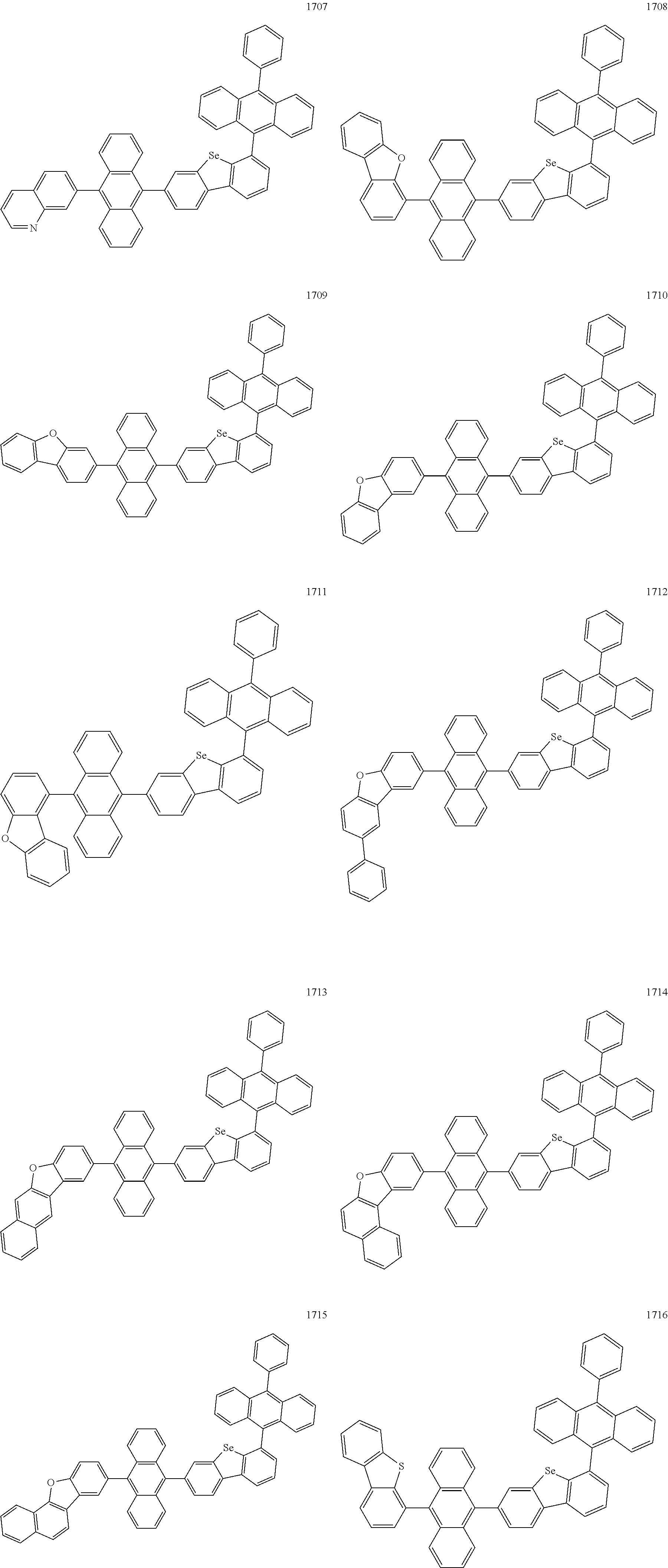

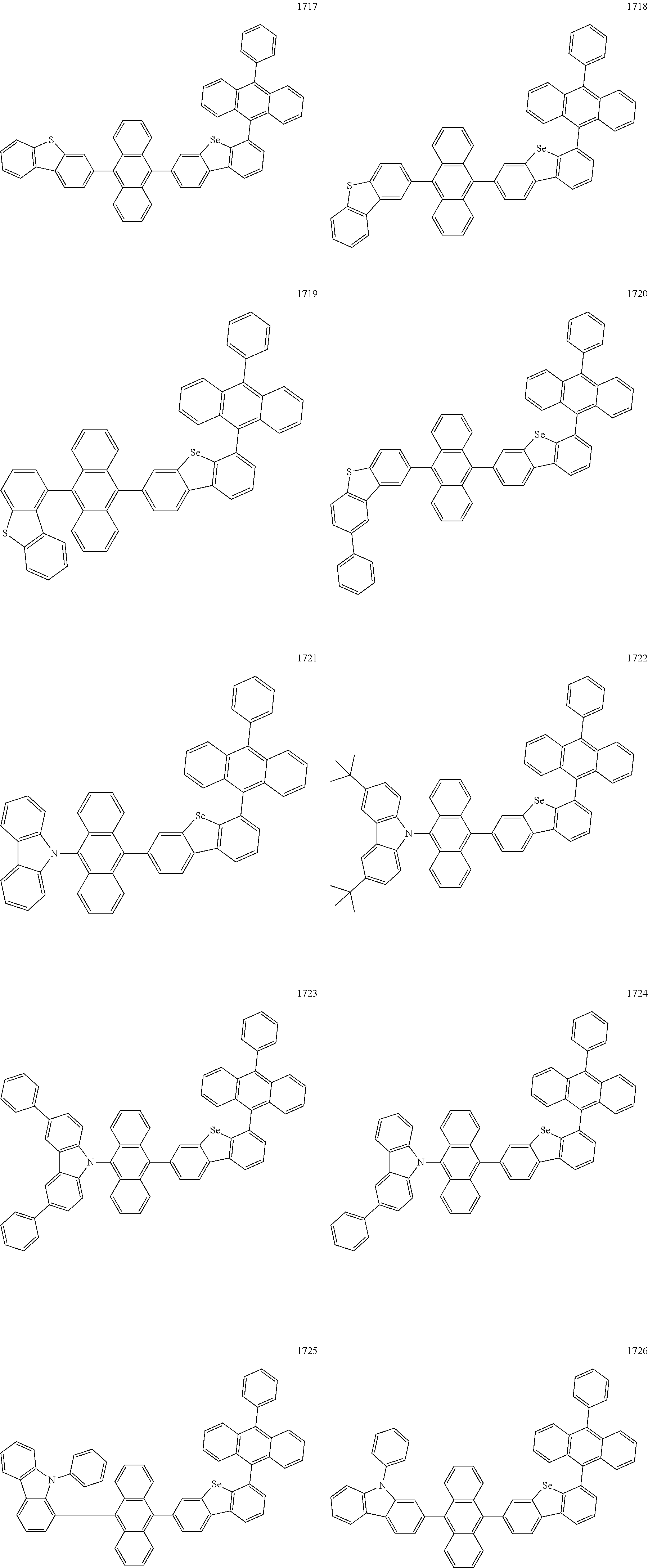

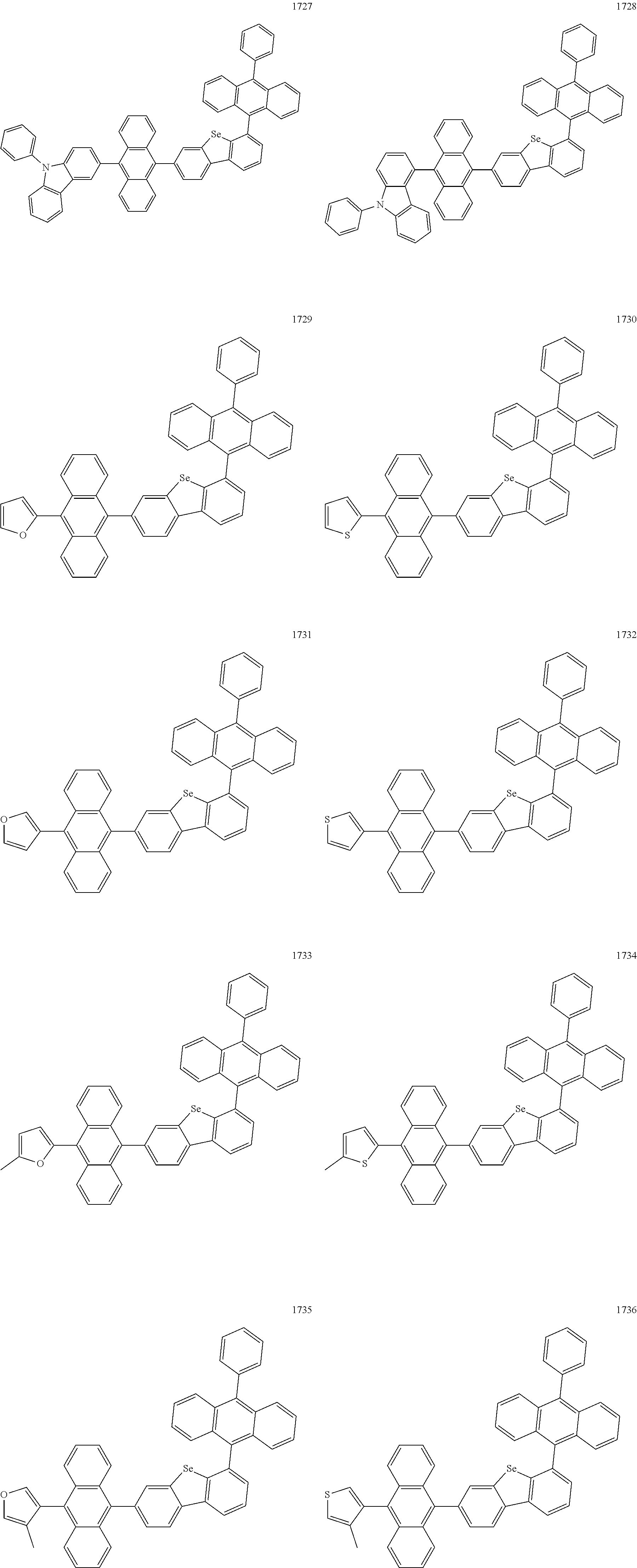

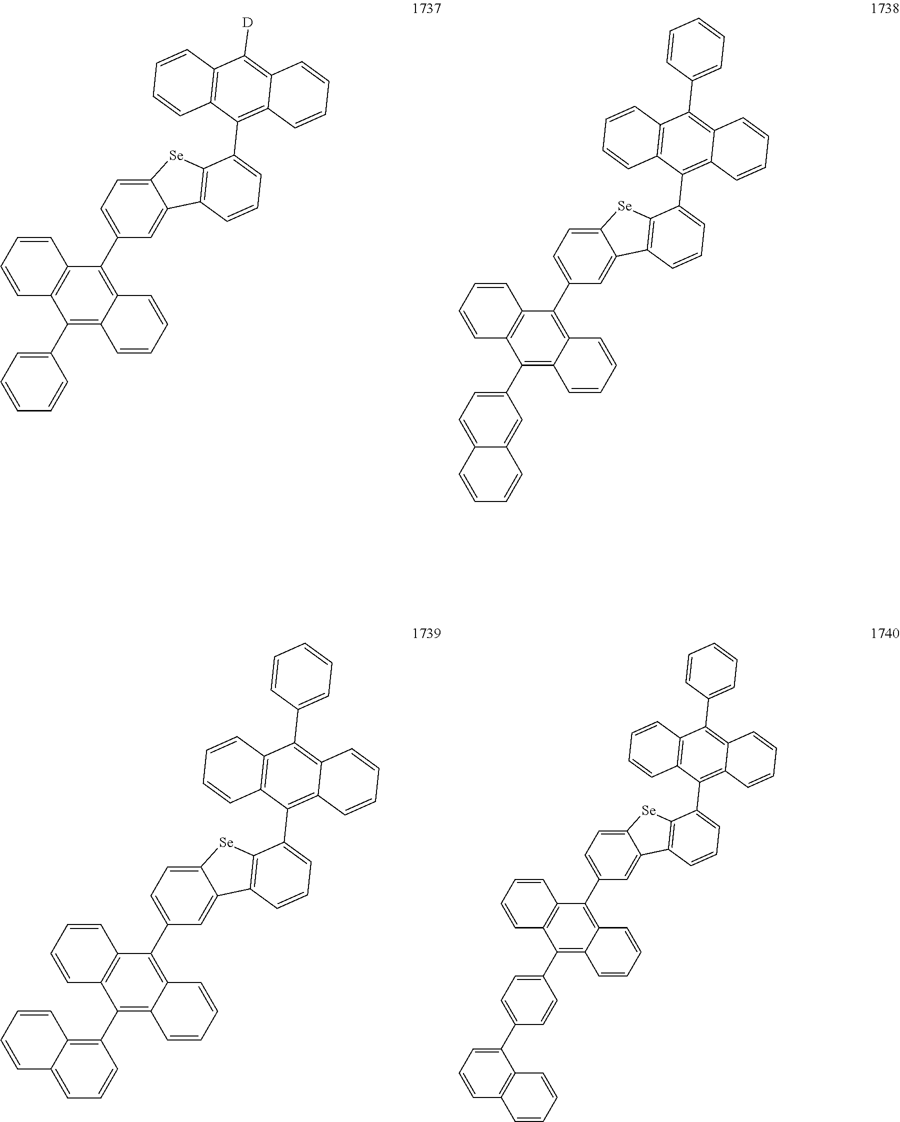 [SMILES: [2H]c1c2ccccc2c(-c2cccc3c2[se]c2ccc(-c4c5ccccc5c(-c5ccccc5)c5ccccc45)cc23)c2ccccc12 ; c1ccc(-c2c3ccccc3c(-c3cccc4c3[se]c3ccc(-c5c6ccccc6c(-c6ccc7ccccc7c6)c6ccccc56)cc34)c3ccccc23)cc1 ; c1ccc(-c2c3ccccc3c(-c3cccc4c3[se]c3ccc(-c5c6ccccc6c(-c6cccc7ccccc67)c6ccccc56)cc34)c3ccccc23)cc1 ; c1ccc(-c2c3ccccc3c(-c3cccc4c3[se]c3ccc(-c5c6ccccc6c(-c6ccc(-c7cccc8ccccc78)cc6)c6ccccc56)cc34)c3ccccc23)cc1]

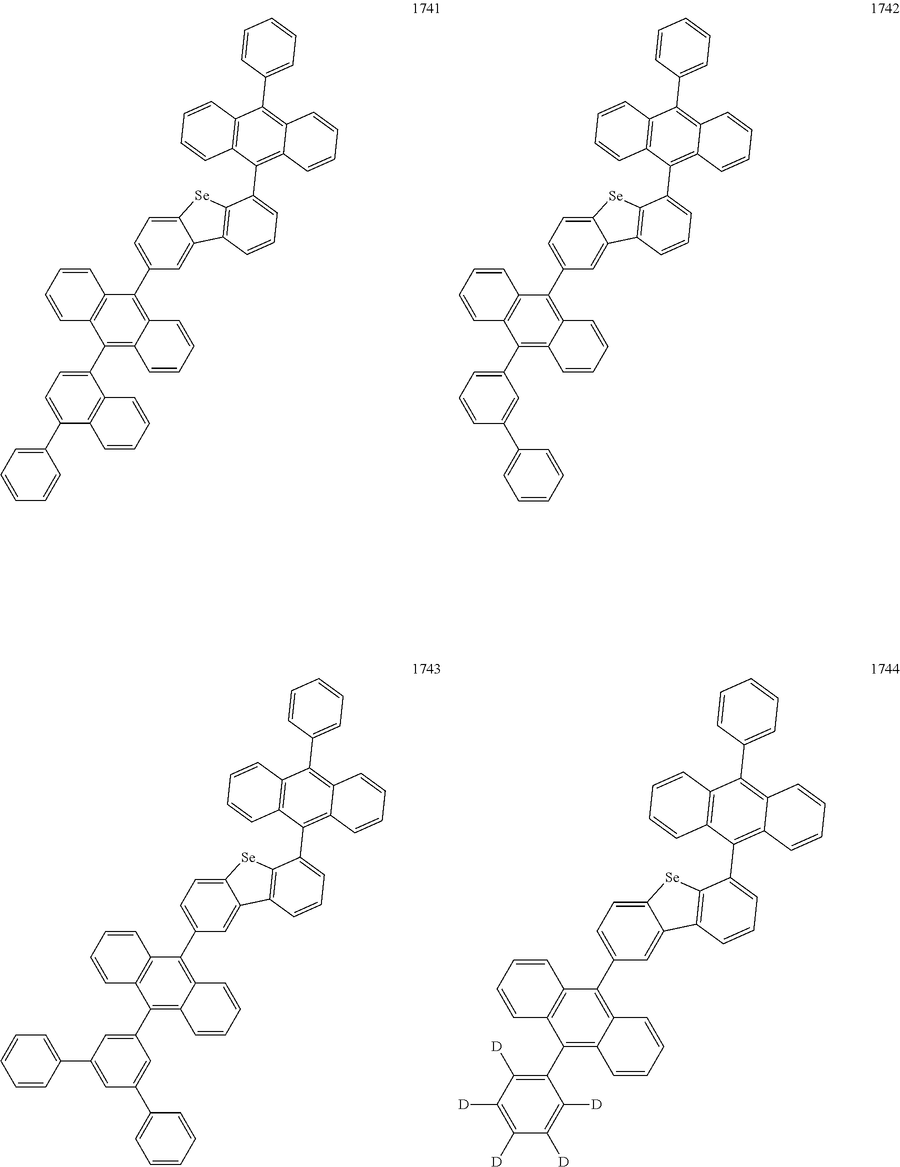 [SMILES: c1ccc(-c2ccc(-c3c4ccccc4c(-c4ccc5[se]c6c(-c7c8ccccc8c(-c8ccccc8)c8ccccc78)cccc6c5c4)c4ccccc34)c3ccccc23)cc1 ; c1ccc(-c2cccc(-c3c4ccccc4c(-c4ccc5[se]c6c(-c7c8ccccc8c(-c8ccccc8)c8ccccc78)cccc6c5c4)c4ccccc34)c2)cc1 ; c1ccc(-c2cc(-c3ccccc3)cc(-c3c4ccccc4c(-c4ccc5[se]c6c(-c7c8ccccc8c(-c8ccccc8)c8ccccc78)cccc6c5c4)c4ccccc34)c2)cc1 ; [2H]c1c([2H])c([2H])c(-c2c3ccccc3c(-c3ccc4[se]c5c(-c6c7ccccc7c(-c7ccccc7)c7ccccc67)cccc5c4c3)c3ccccc23)c([2H])c1[2H]]

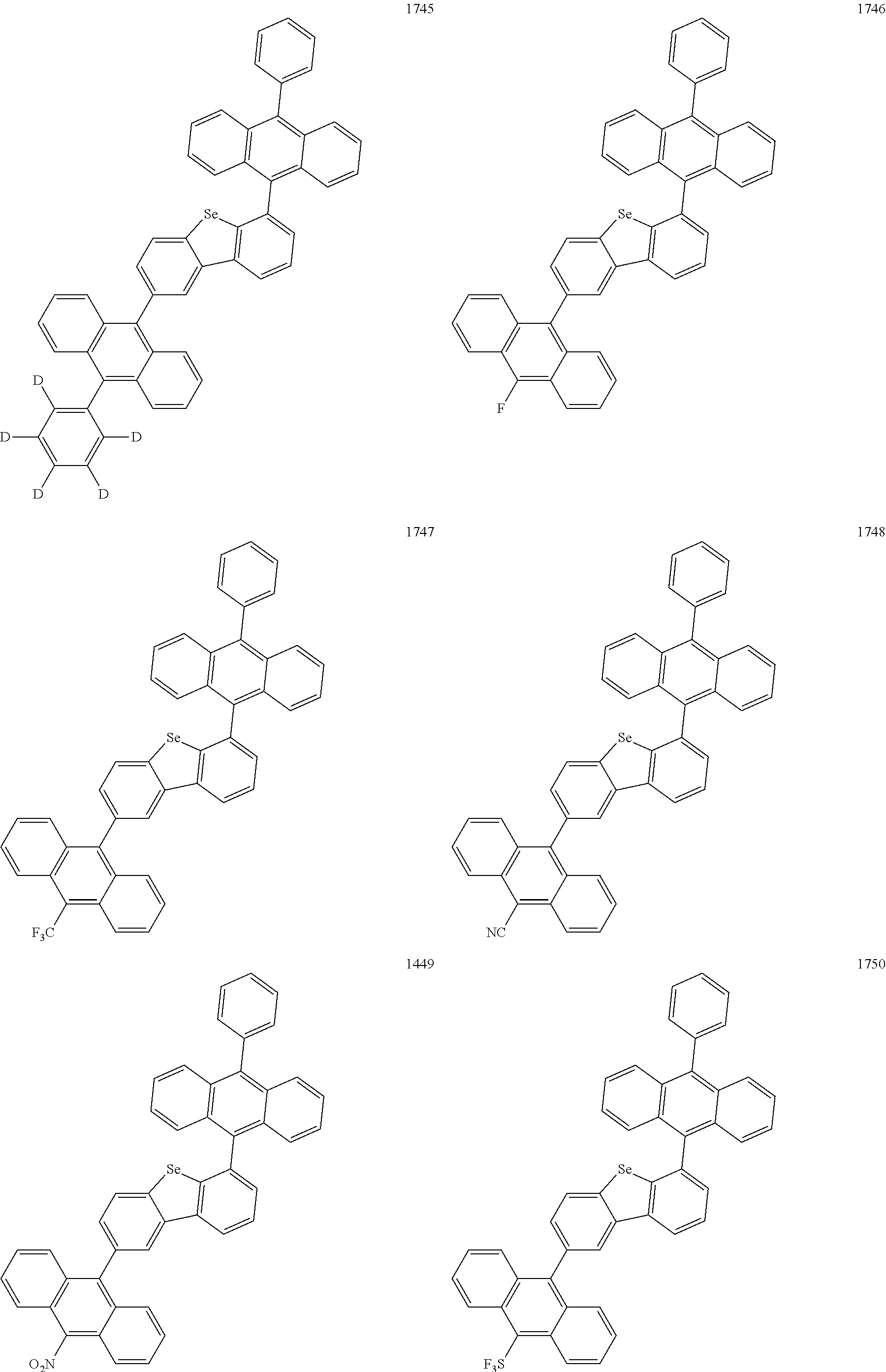

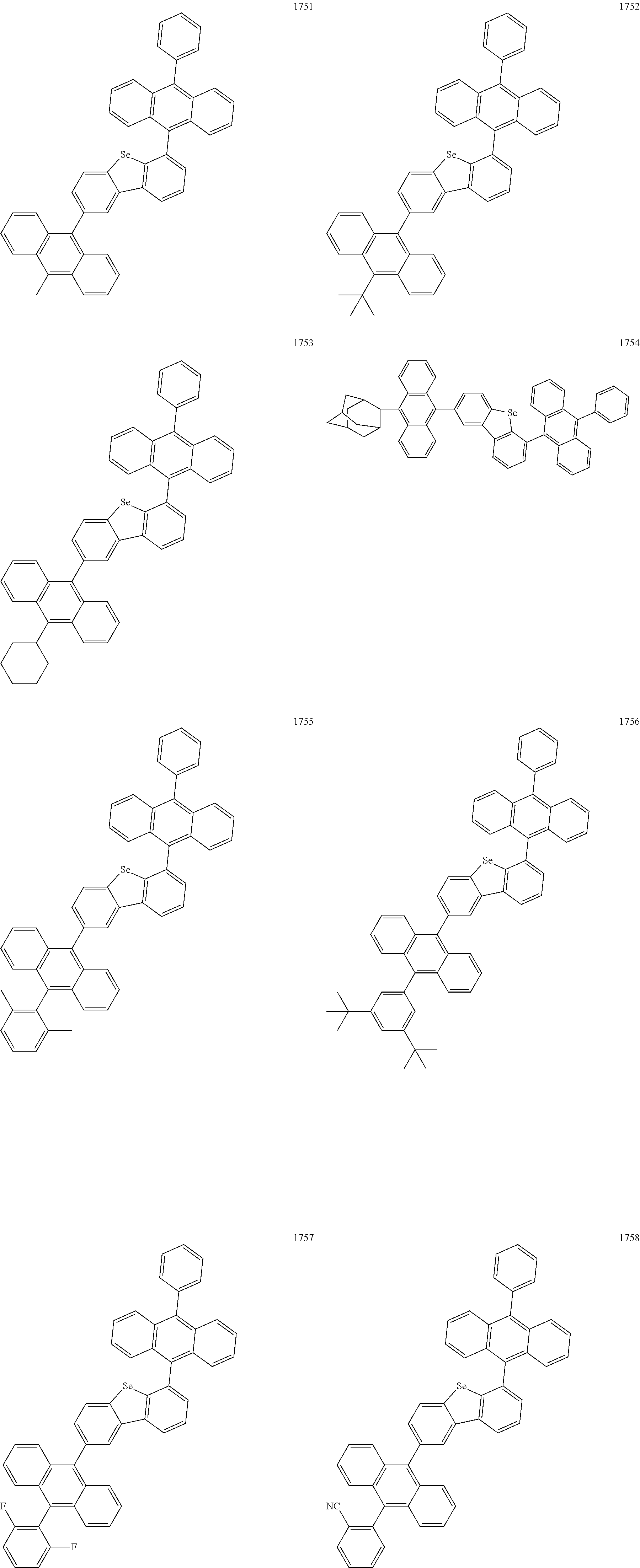

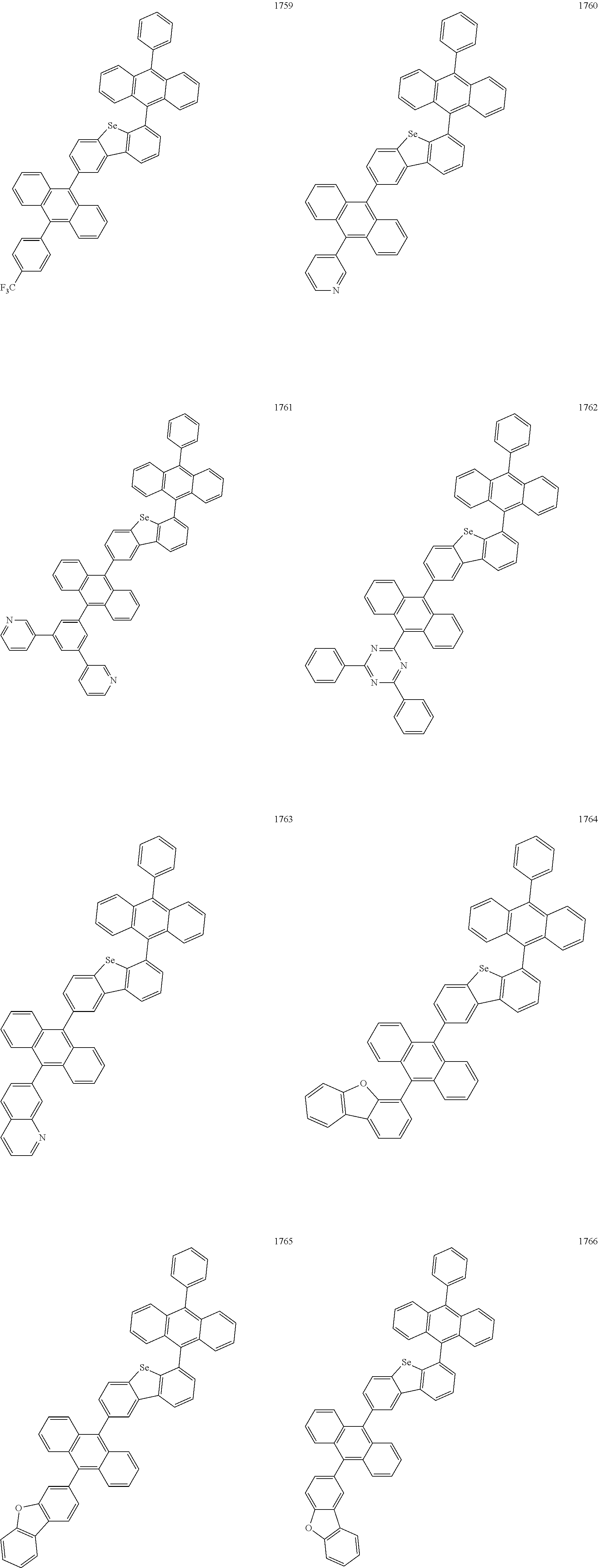

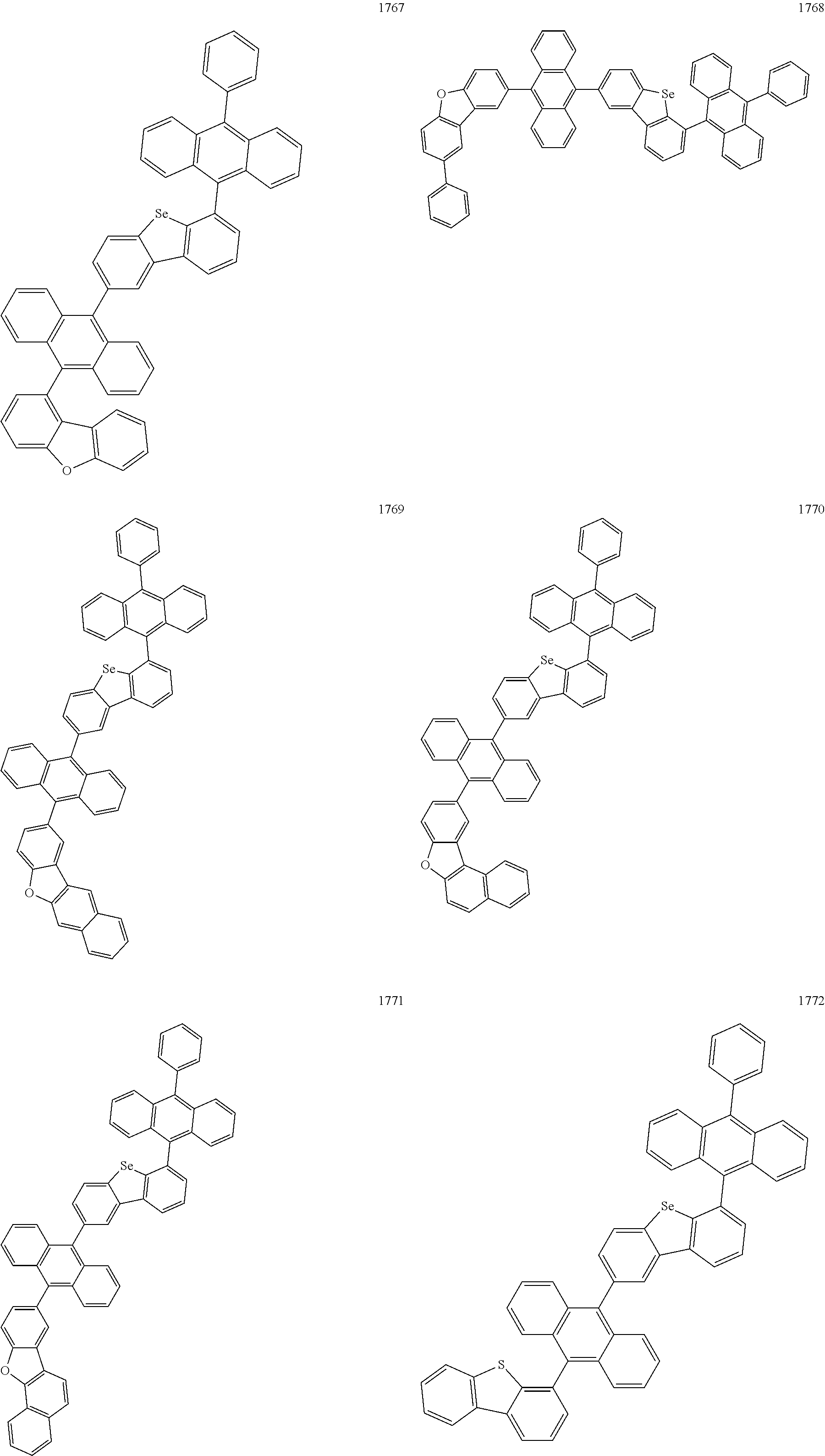

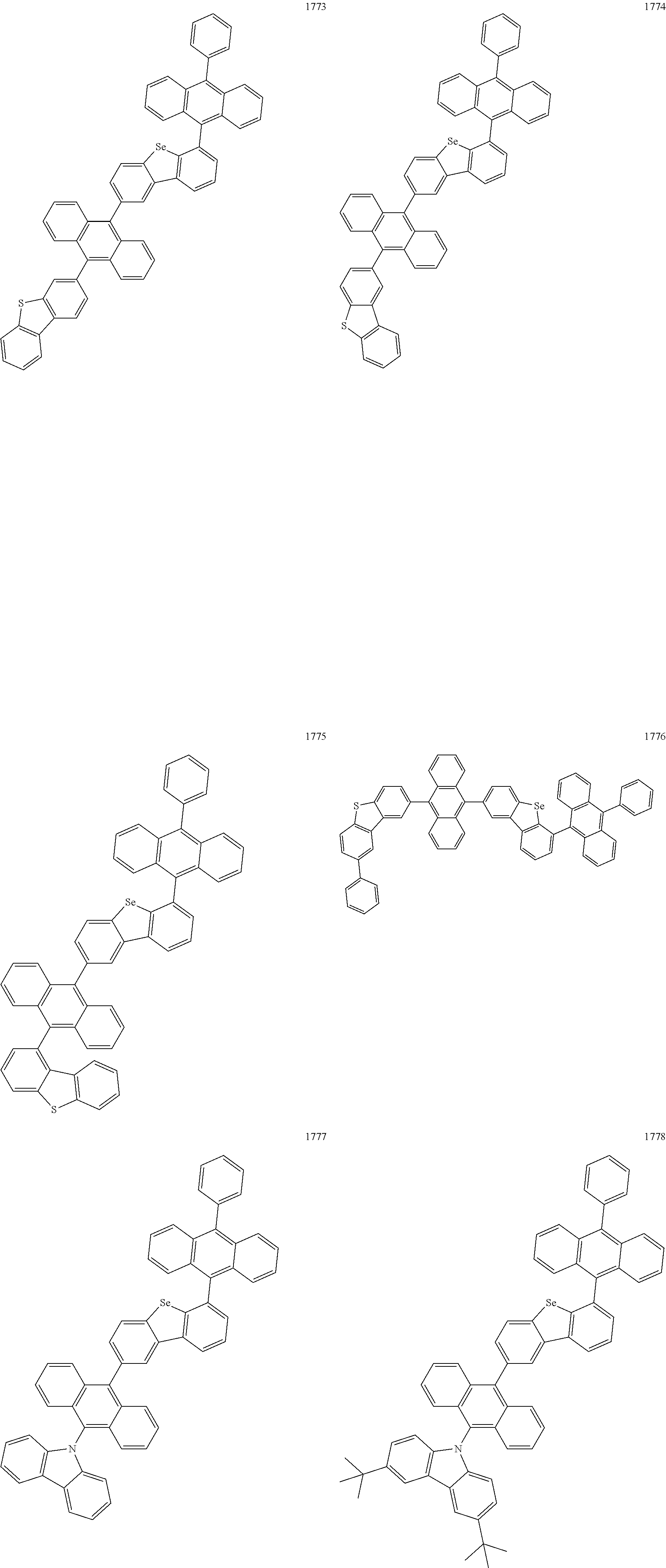

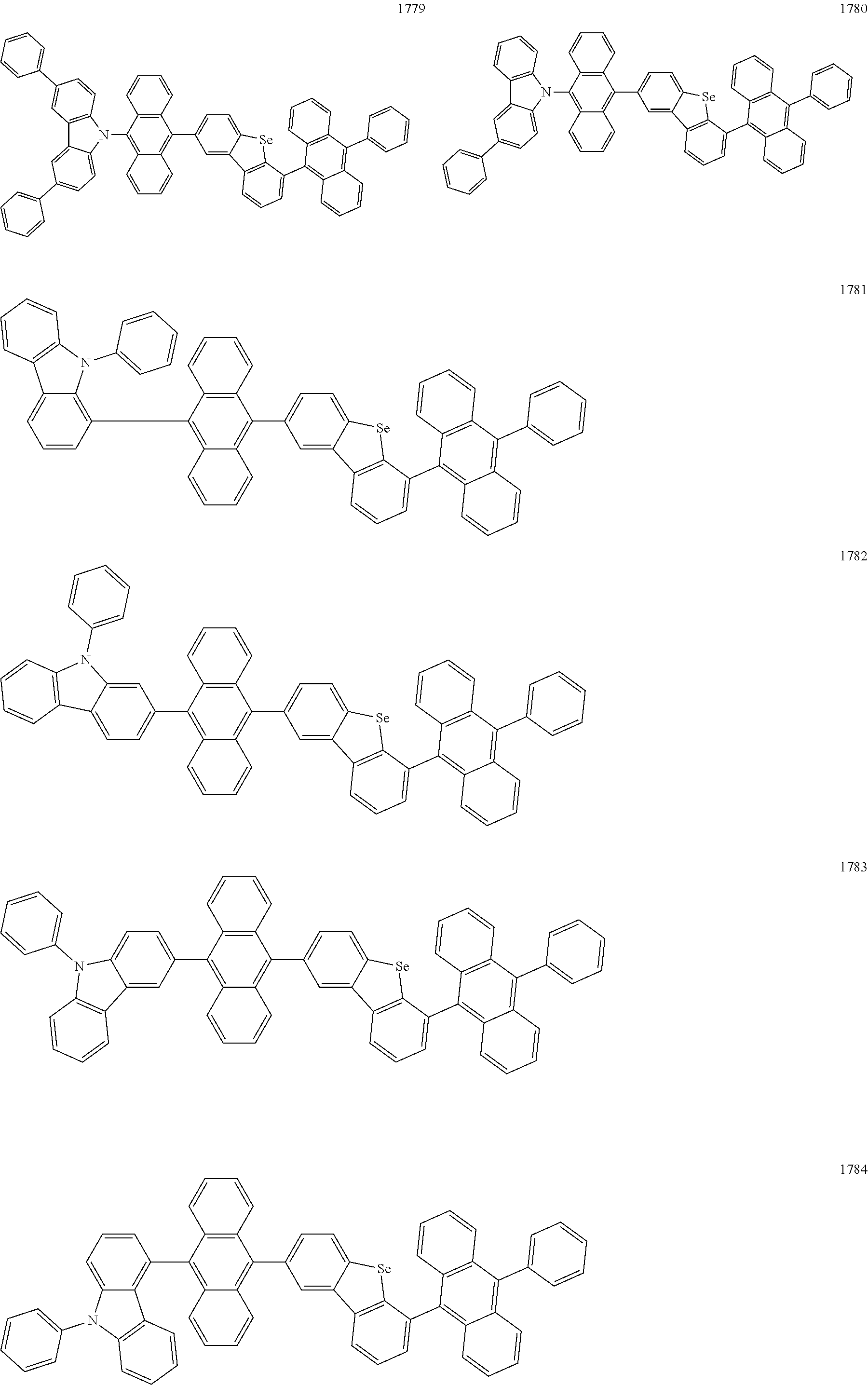

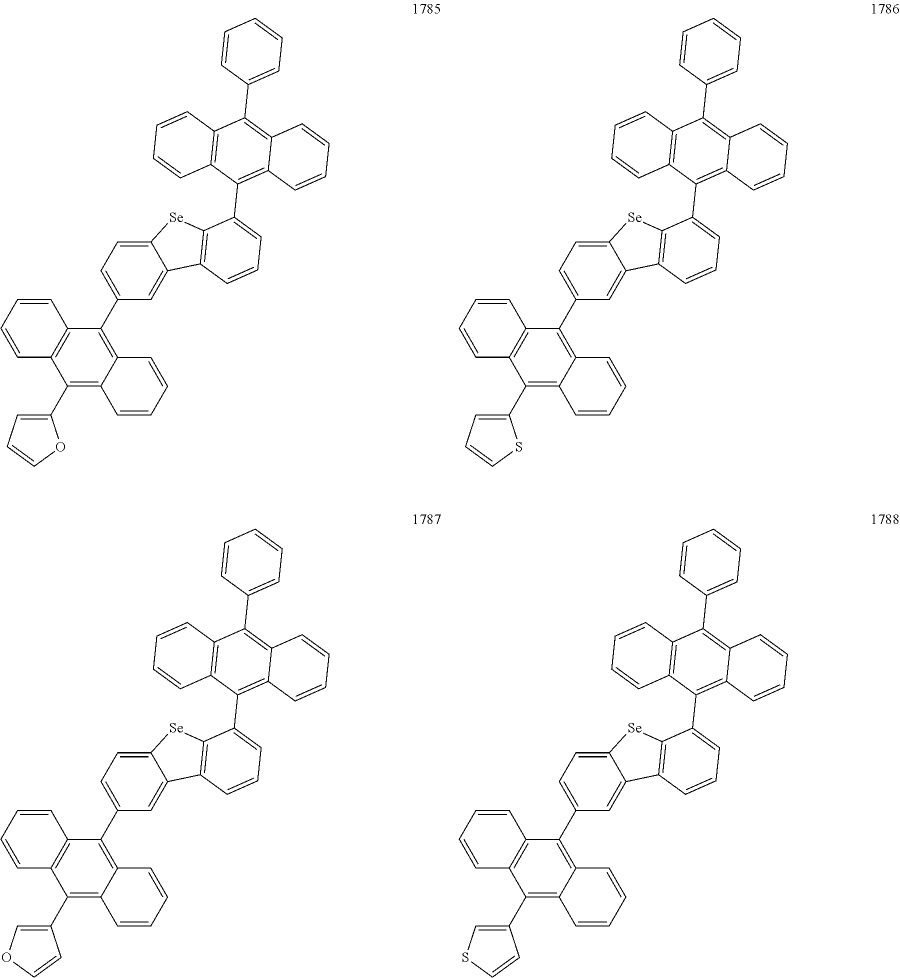 [SMILES: c1ccc(-c2c3ccccc3c(-c3cccc4c3[se]c3ccc(-c5c6ccccc6c(-c6ccco6)c6ccccc56)cc34)c3ccccc23)cc1 ; c1ccc(-c2c3ccccc3c(-c3cccc4c3[se]c3ccc(-c5c6ccccc6c(-c6cccs6)c6ccccc56)cc34)c3ccccc23)cc1 ; c1ccc(-c2c3ccccc3c(-c3cccc4c3[se]c3ccc(-c5c6ccccc6c(-c6ccoc6)c6ccccc56)cc34)c3ccccc23)cc1 ; c1ccc(-c2c3ccccc3c(-c3cccc4c3[se]c3ccc(-c5c6ccccc6c(-c6ccsc6)c6ccccc56)cc34)c3ccccc23)cc1]

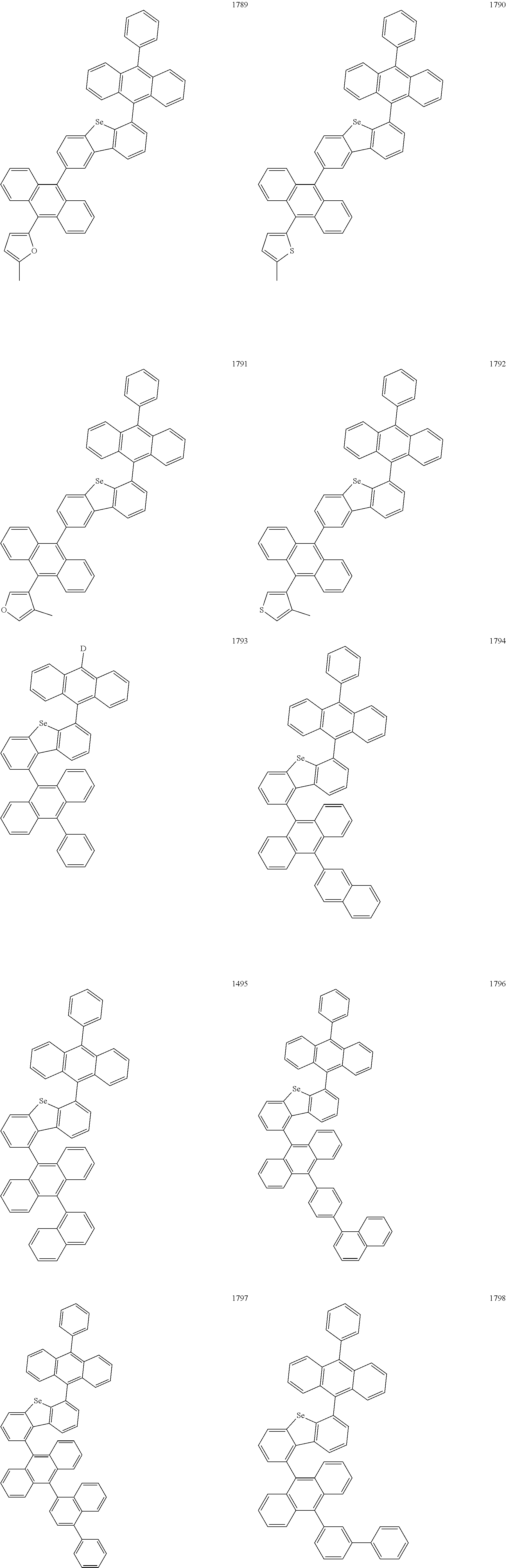

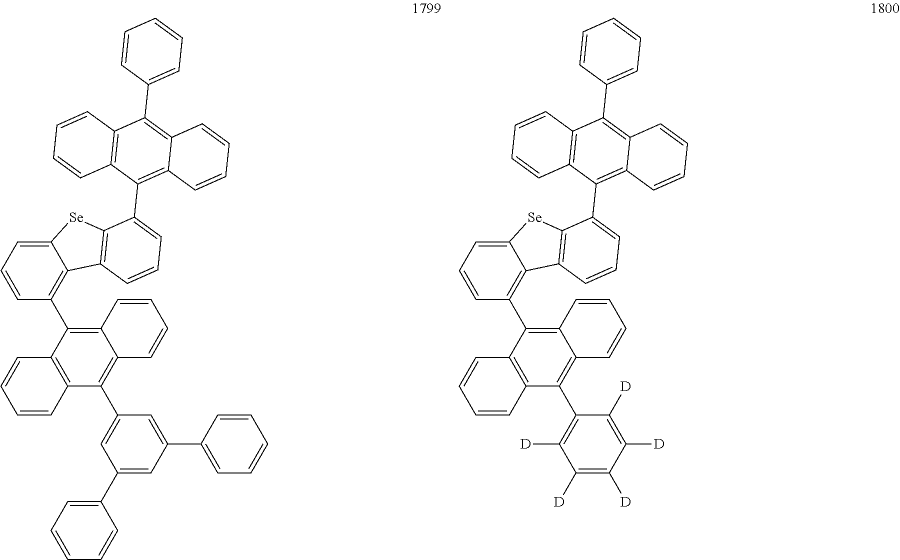 [SMILES: c1ccc(-c2cc(-c3ccccc3)cc(-c3c4ccccc4c(-c4cccc5[se]c6c(-c7c8ccccc8c(-c8ccccc8)c8ccccc78)cccc6c45)c4ccccc34)c2)cc1 ; [2H]c1c([2H])c([2H])c(-c2c3ccccc3c(-c3cccc4[se]c5c(-c6c7ccccc7c(-c7ccccc7)c7ccccc67)cccc5c34)c3ccccc23)c([2H])c1[2H]]

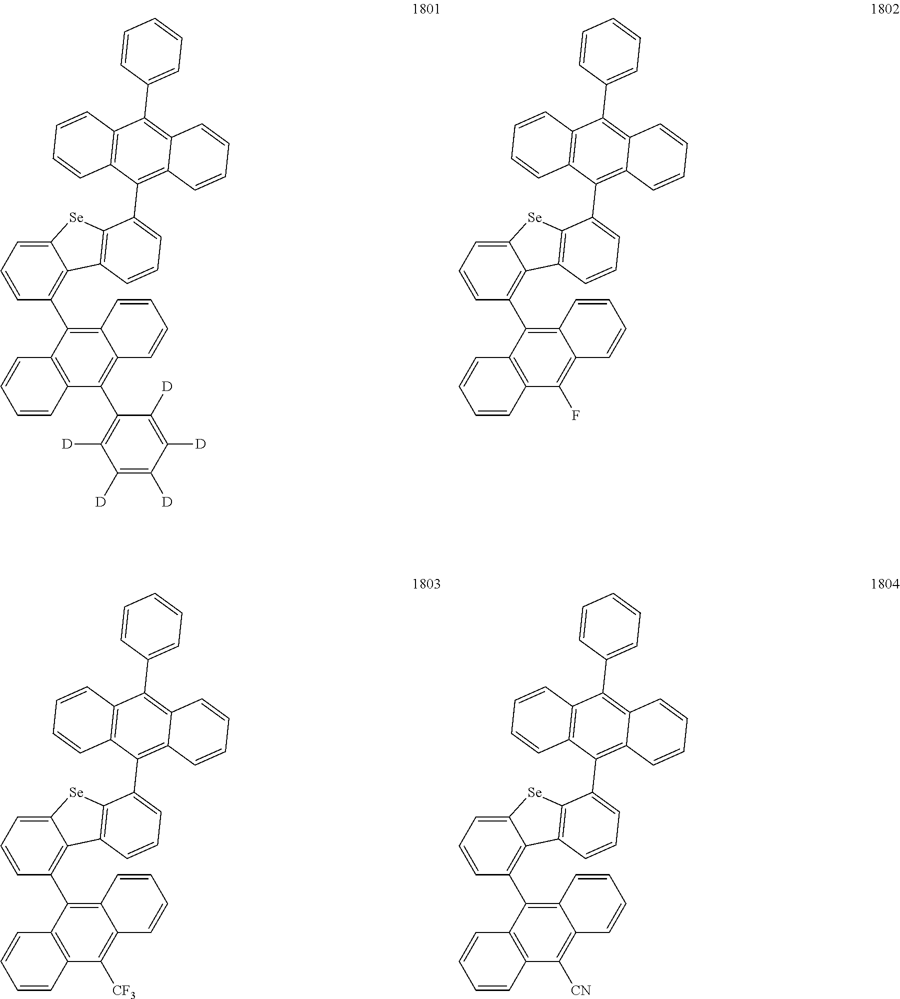 [SMILES: [2H]c1c([2H])c([2H])c(-c2c3ccccc3c(-c3cccc4[se]c5c(-c6c7ccccc7c(-c7ccccc7)c7ccccc67)cccc5c34)c3ccccc23)c([2H])c1[2H] ; Fc1c2ccccc2c(-c2cccc3[se]c4c(-c5c6ccccc6c(-c6ccccc6)c6ccccc56)cccc4c23)c2ccccc12 ; FC(F)(F)c1c2ccccc2c(-c2cccc3[se]c4c(-c5c6ccccc6c(-c6ccccc6)c6ccccc56)cccc4c23)c2ccccc12 ; N#Cc1c2ccccc2c(-c2cccc3[se]c4c(-c5c6ccccc6c(-c6ccccc6)c6ccccc56)cccc4c23)c2ccccc12]

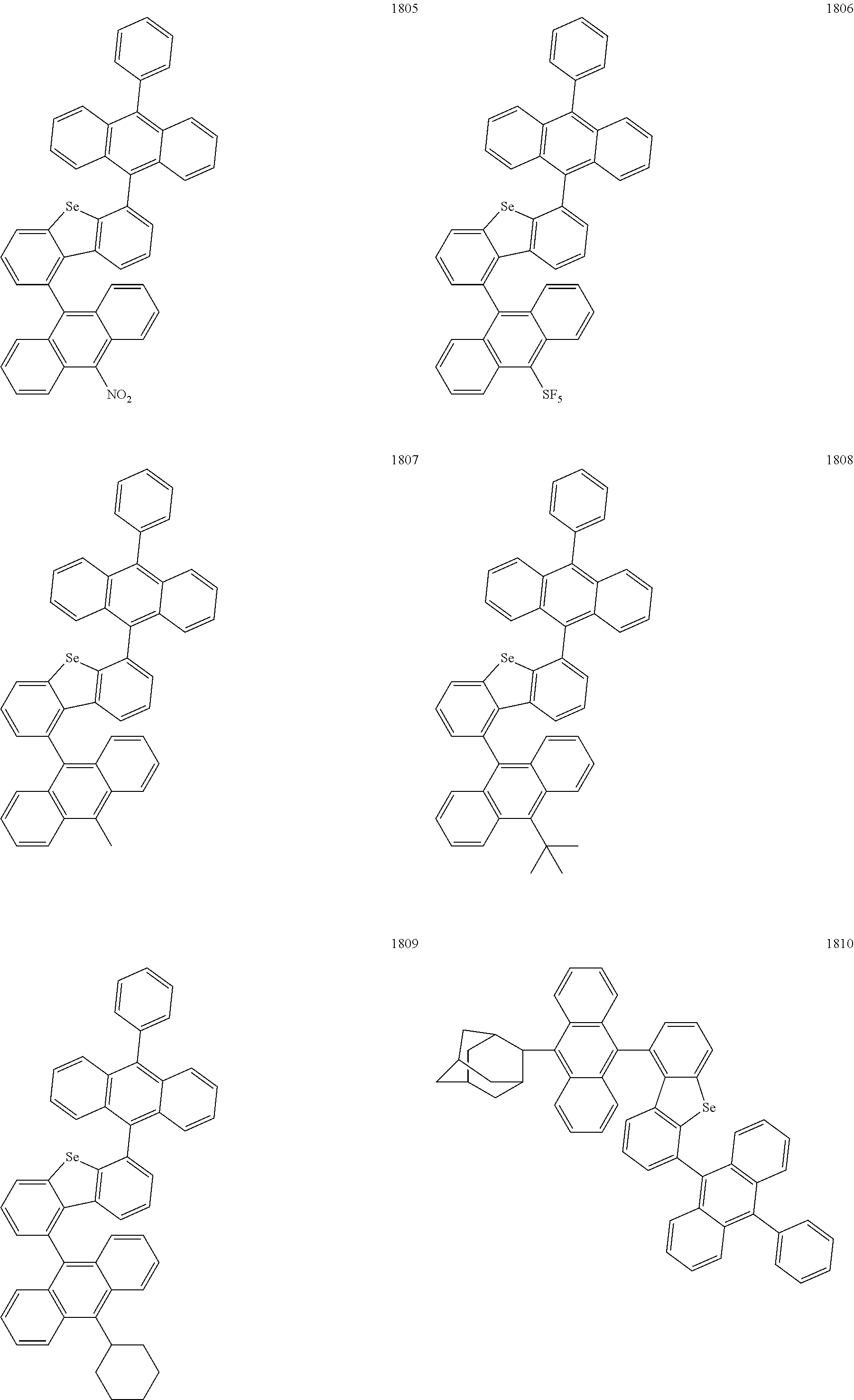

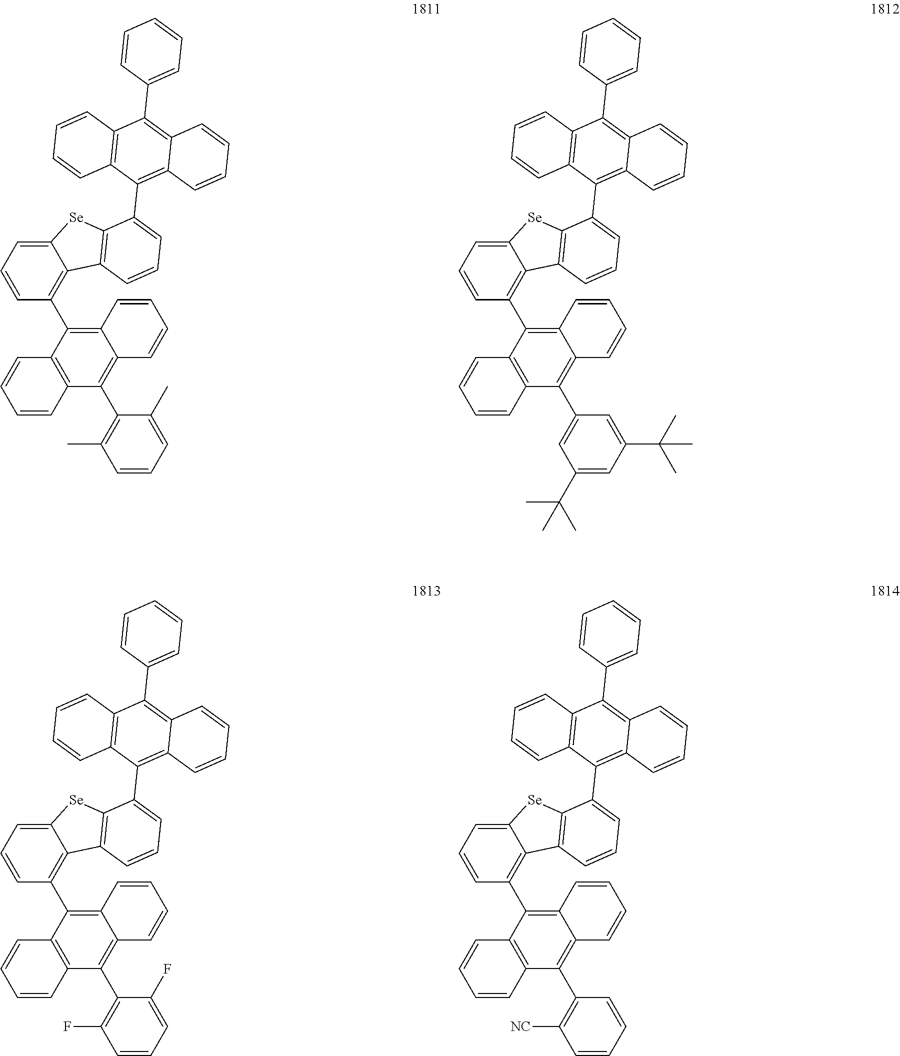 [SMILES: Cc1cccc(C)c1-c1c2ccccc2c(-c2cccc3[se]c4c(-c5c6ccccc6c(-c6ccccc6)c6ccccc56)cccc4c23)c2ccccc12 ; CC(C)(C)c1cc(-c2c3ccccc3c(-c3cccc4[se]c5c(-c6c7ccccc7c(-c7ccccc7)c7ccccc67)cccc5c34)c3ccccc23)cc(C(C)(C)C)c1 ; Fc1cccc(F)c1-c1c2ccccc2c(-c2cccc3[se]c4c(-c5c6ccccc6c(-c6ccccc6)c6ccccc56)cccc4c23)c2ccccc12 ; N#Cc1ccccc1-c1c2ccccc2c(-c2cccc3[se]c4c(-c5c6ccccc6c(-c6ccccc6)c6ccccc56)cccc4c23)c2ccccc12]

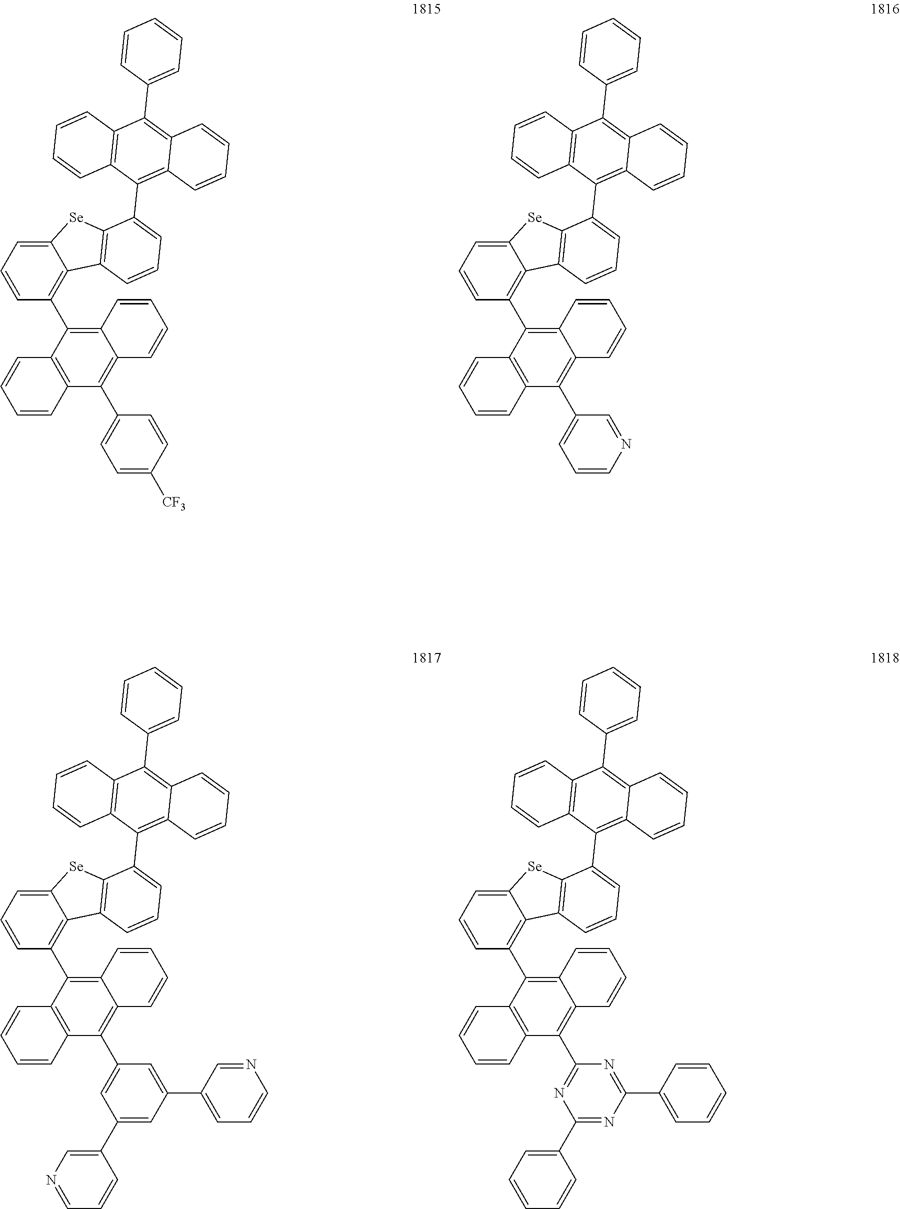 [SMILES: FC(F)(F)c1ccc(-c2c3ccccc3c(-c3cccc4[se]c5c(-c6c7ccccc7c(-c7ccccc7)c7ccccc67)cccc5c34)c3ccccc23)cc1 ; c1ccc(-c2c3ccccc3c(-c3cccc4c3[se]c3cccc(-c5c6ccccc6c(-c6cccnc6)c6ccccc56)c34)c3ccccc23)cc1 ; c1ccc(-c2c3ccccc3c(-c3cccc4c3[se]c3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5[se]c6c(-c7c8ccccc8c(-c8ccccc8)c8ccccc78)cccc6c45)c4ccccc34)n2)cc1]

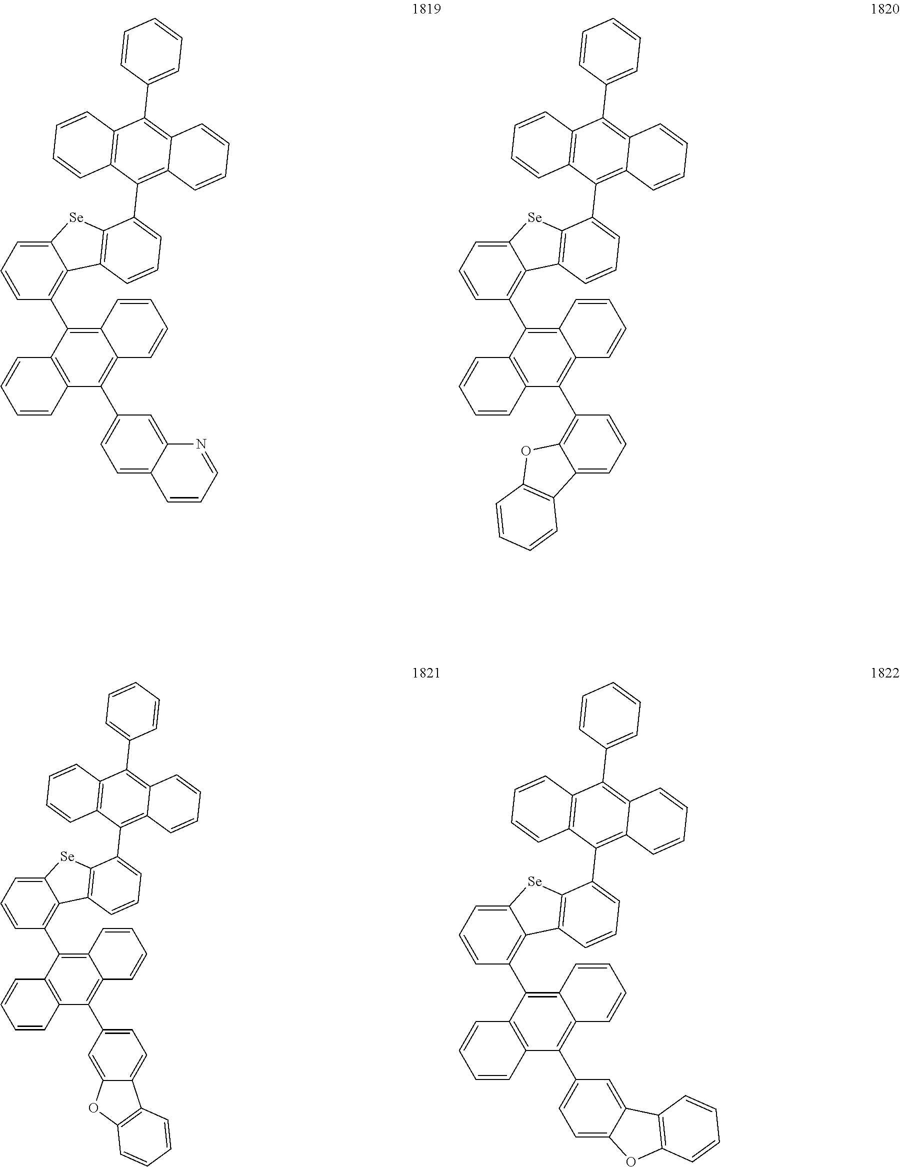 [SMILES: c1ccc(-c2c3ccccc3c(-c3cccc4c3[se]c3cccc(-c5c6ccccc6c(-c6ccc7cccnc7c6)c6ccccc56)c34)c3ccccc23)cc1 ; c1ccc(-c2c3ccccc3c(-c3cccc4c3[se]c3cccc(-c5c6ccccc6c(-c6cccc7c6oc6ccccc67)c6ccccc56)c34)c3ccccc23)cc1 ; c1ccc(-c2c3ccccc3c(-c3cccc4c3[se]c3cccc(-c5c6ccccc6c(-c6ccc7c(c6)oc6ccccc67)c6ccccc56)c34)c3ccccc23)cc1 ; c1ccc(-c2c3ccccc3c(-c3cccc4c3[se]c3cccc(-c5c6ccccc6c(-c6ccc7oc8ccccc8c7c6)c6ccccc56)c34)c3ccccc23)cc1]

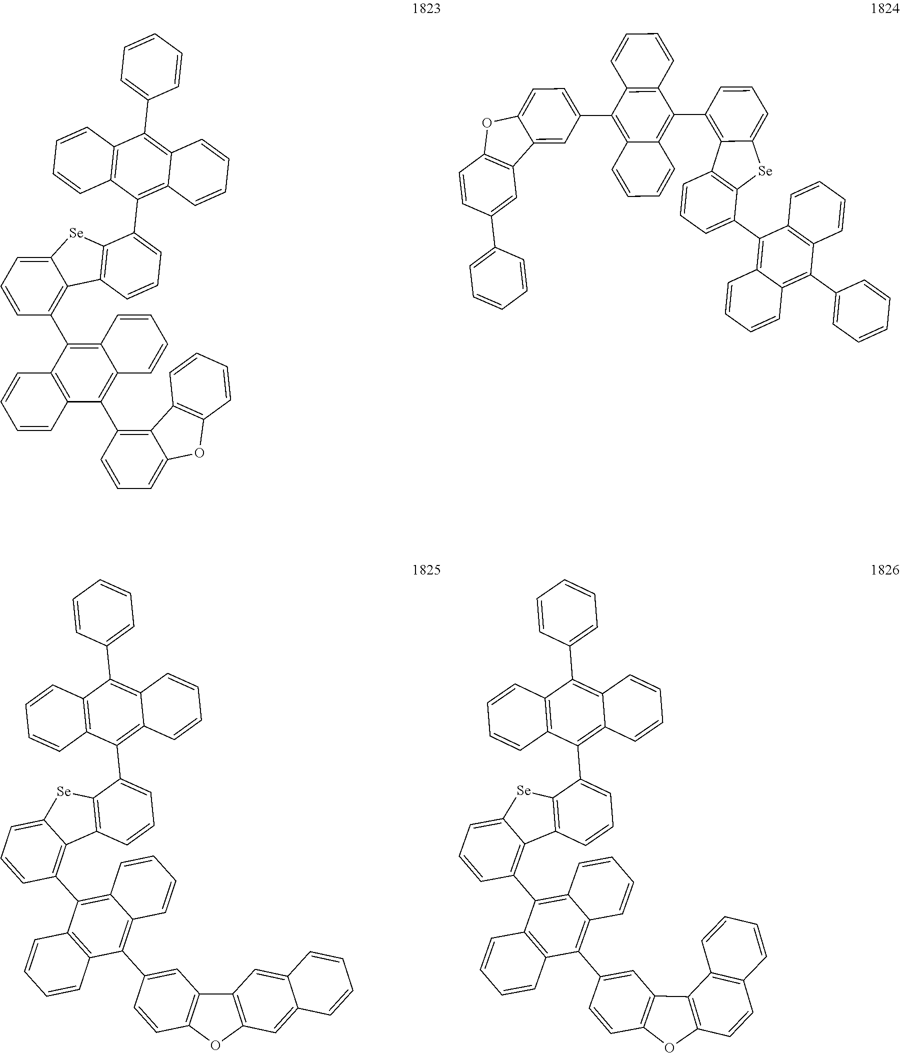 [SMILES: c1ccc(-c2c3ccccc3c(-c3cccc4c3[se]c3cccc(-c5c6ccccc6c(-c6cccc7oc8ccccc8c67)c6ccccc56)c34)c3ccccc23)cc1 ; c1ccc(-c2ccc3oc4ccc(-c5c6ccccc6c(-c6cccc7[se]c8c(-c9c%10ccccc%10c(-c%10ccccc%10)c%10ccccc9%10)cccc8c67)c6ccccc56)cc4c3c2)cc1 ; c1ccc(-c2c3ccccc3c(-c3cccc4c3[se]c3cccc(-c5c6ccccc6c(-c6ccc7oc8cc9ccccc9cc8c7c6)c6ccccc56)c34)c3ccccc23)cc1 ; c1ccc(-c2c3ccccc3c(-c3cccc4c3[se]c3cccc(-c5c6ccccc6c(-c6ccc7oc8ccc9ccccc9c8c7c6)c6ccccc56)c34)c3ccccc23)cc1]

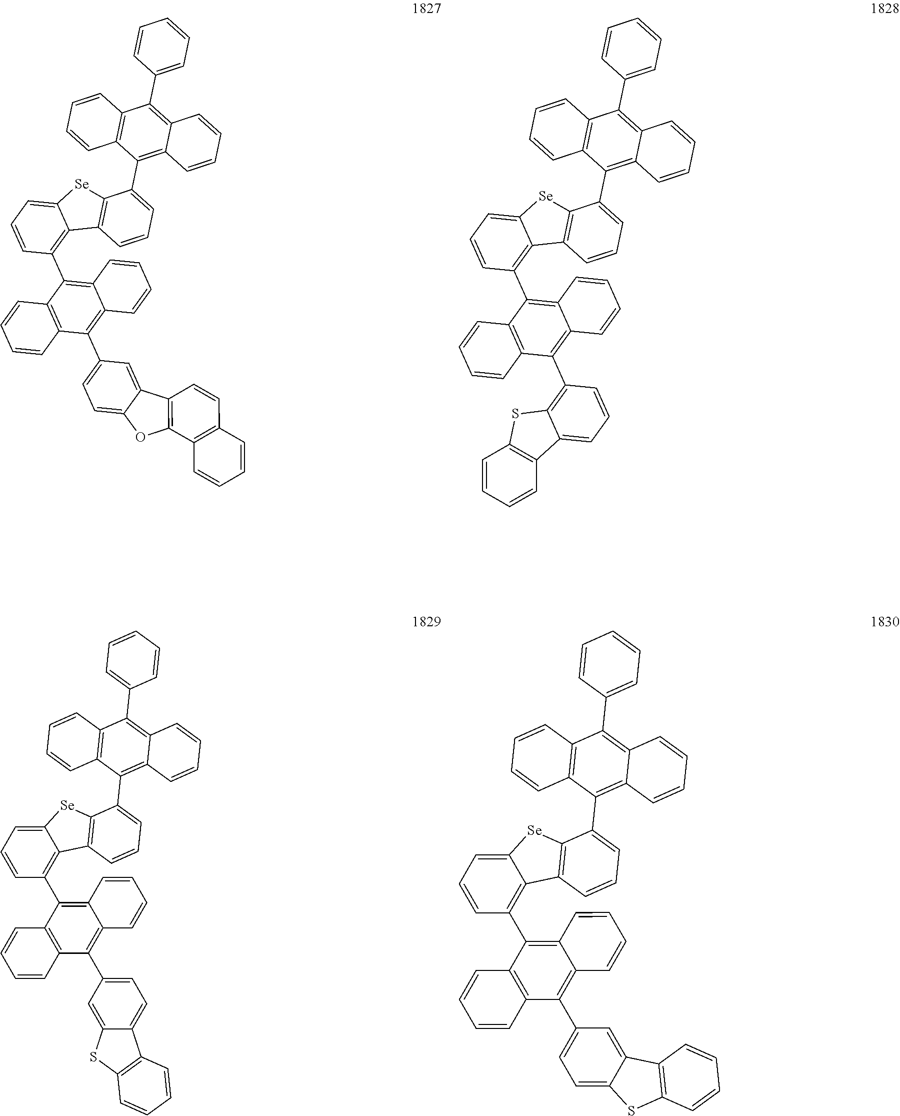 [SMILES: c1ccc(-c2c3ccccc3c(-c3cccc4c3[se]c3cccc(-c5c6ccccc6c(-c6ccc7oc8c9ccccc9ccc8c7c6)c6ccccc56)c34)c3ccccc23)cc1 ; c1ccc(-c2c3ccccc3c(-c3cccc4c3[se]c3cccc(-c5c6ccccc6c(-c6cccc7c6sc6ccccc67)c6ccccc56)c34)c3ccccc23)cc1 ; c1ccc(-c2c3ccccc3c(-c3cccc4c3[se]c3cccc(-c5c6ccccc6c(-c6ccc7c(c6)sc6ccccc67)c6ccccc56)c34)c3ccccc23)cc1 ; c1ccc(-c2c3ccccc3c(-c3cccc4c3[se]c3cccc(-c5c6ccccc6c(-c6ccc7sc8ccccc8c7c6)c6ccccc56)c34)c3ccccc23)cc1]

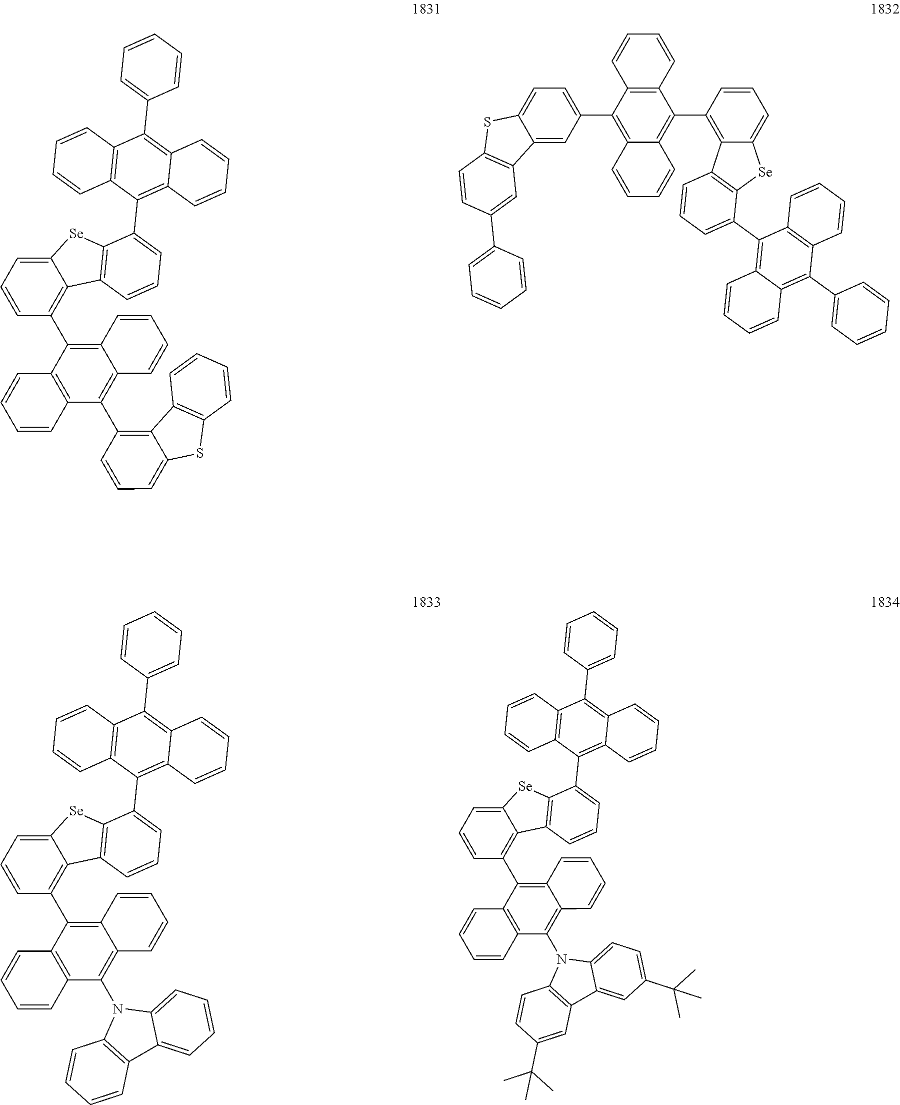 [SMILES: c1ccc(-c2c3ccccc3c(-c3cccc4c3[se]c3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7[se]c8c(-c9c%10ccccc%10c(-c%10ccccc%10)c%10ccccc9%10)cccc8c67)c6ccccc56)cc4c3c2)cc1 ; c1ccc(-c2c3ccccc3c(-c3cccc4c3[se]c3cccc(-c5c6ccccc6c(-n6c7ccccc7c7ccccc76)c6ccccc56)c34)c3ccccc23)cc1 ; CC(C)(C)c1ccc2c(c1)c1cc(C(C)(C)C)ccc1n2-c1c2ccccc2c(-c2cccc3[se]c4c(-c5c6ccccc6c(-c6ccccc6)c6ccccc56)cccc4c23)c2ccccc12]

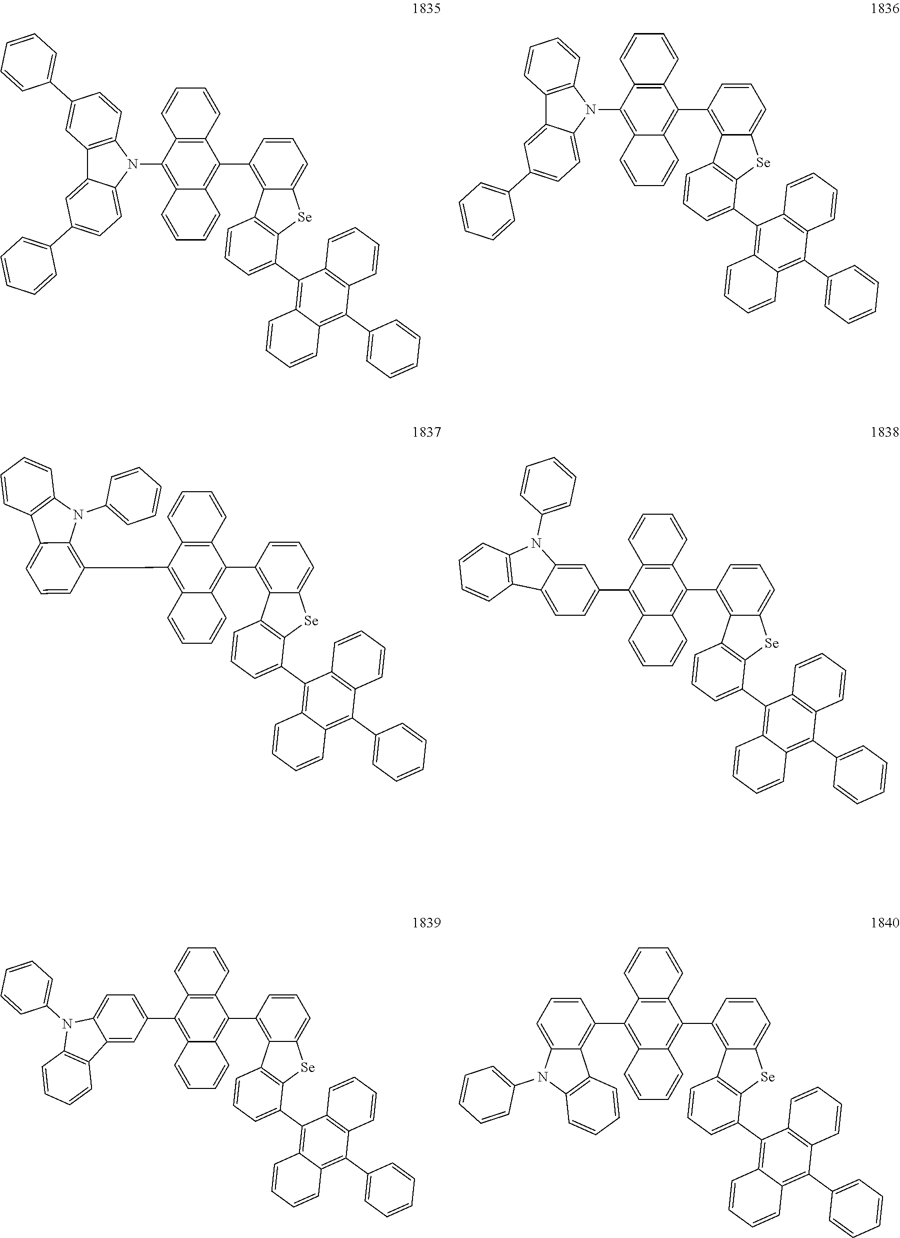

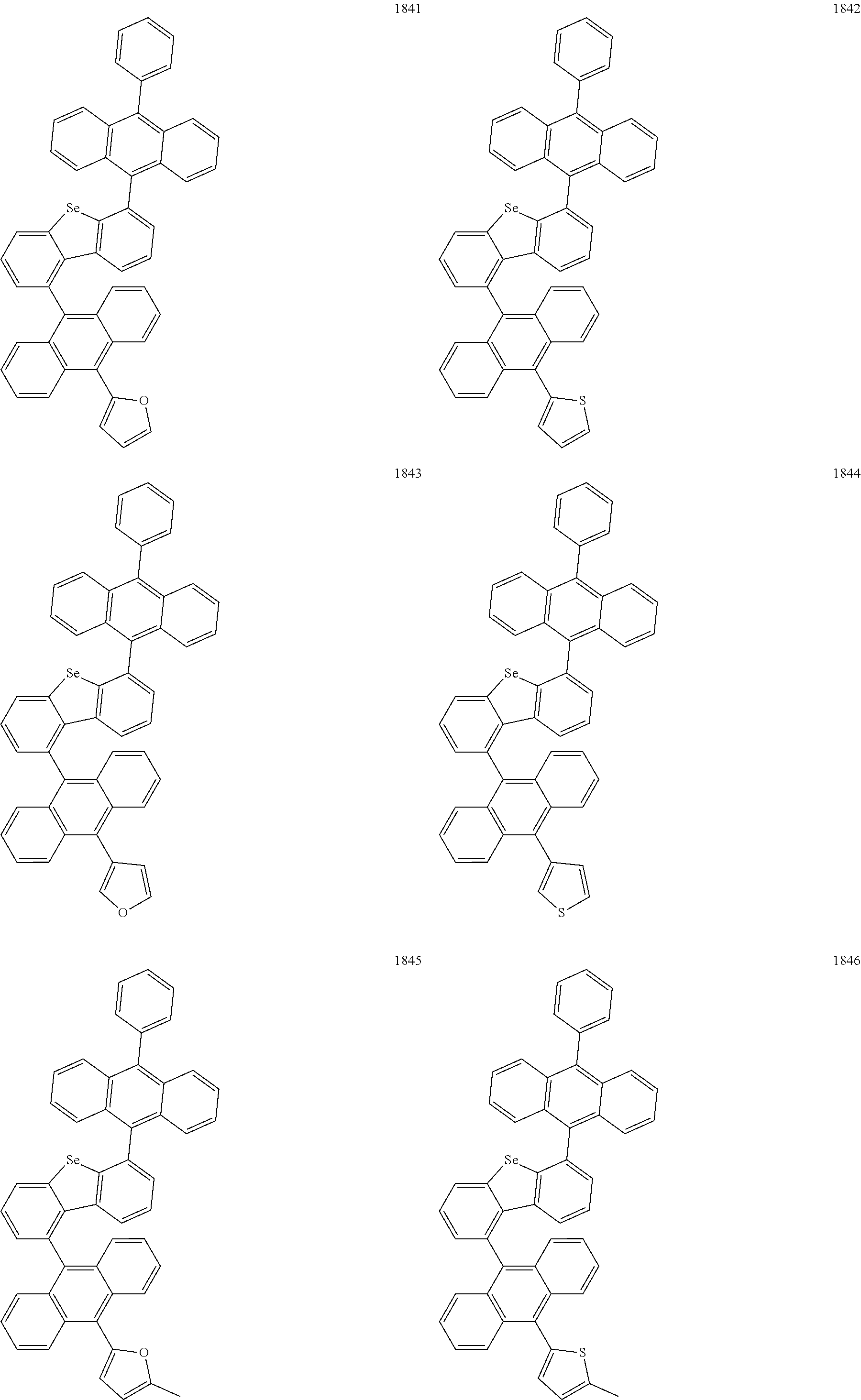

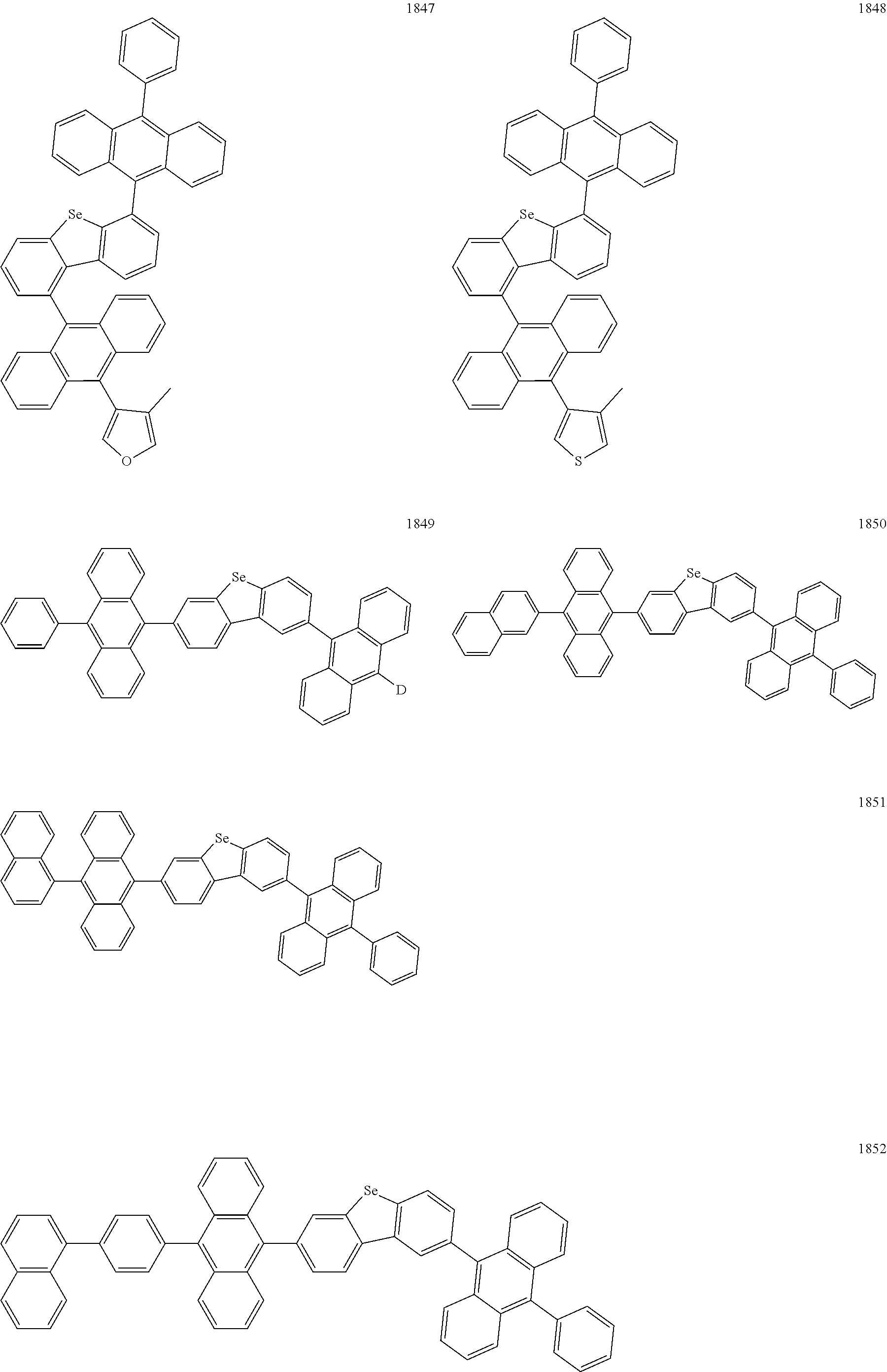

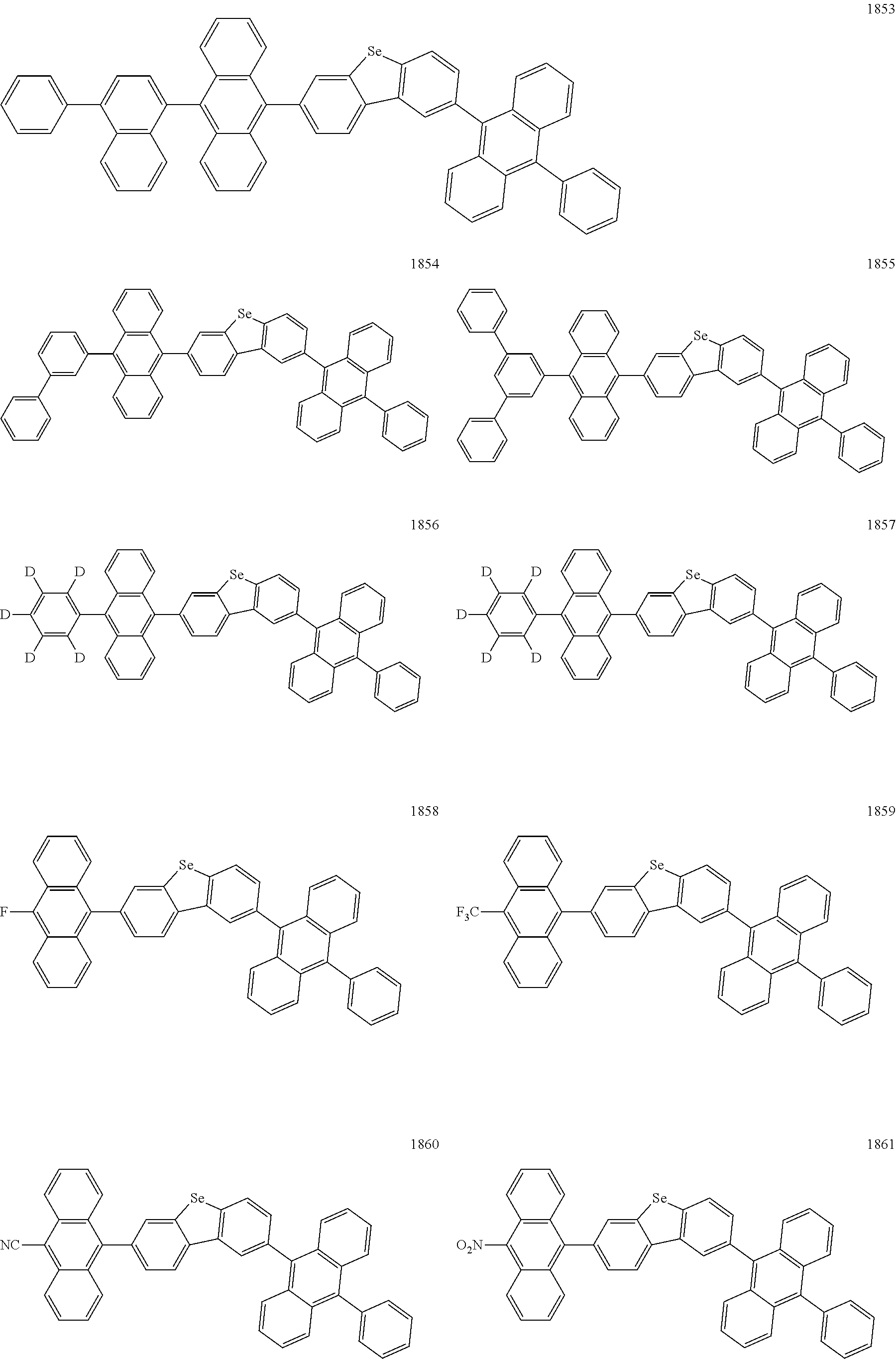

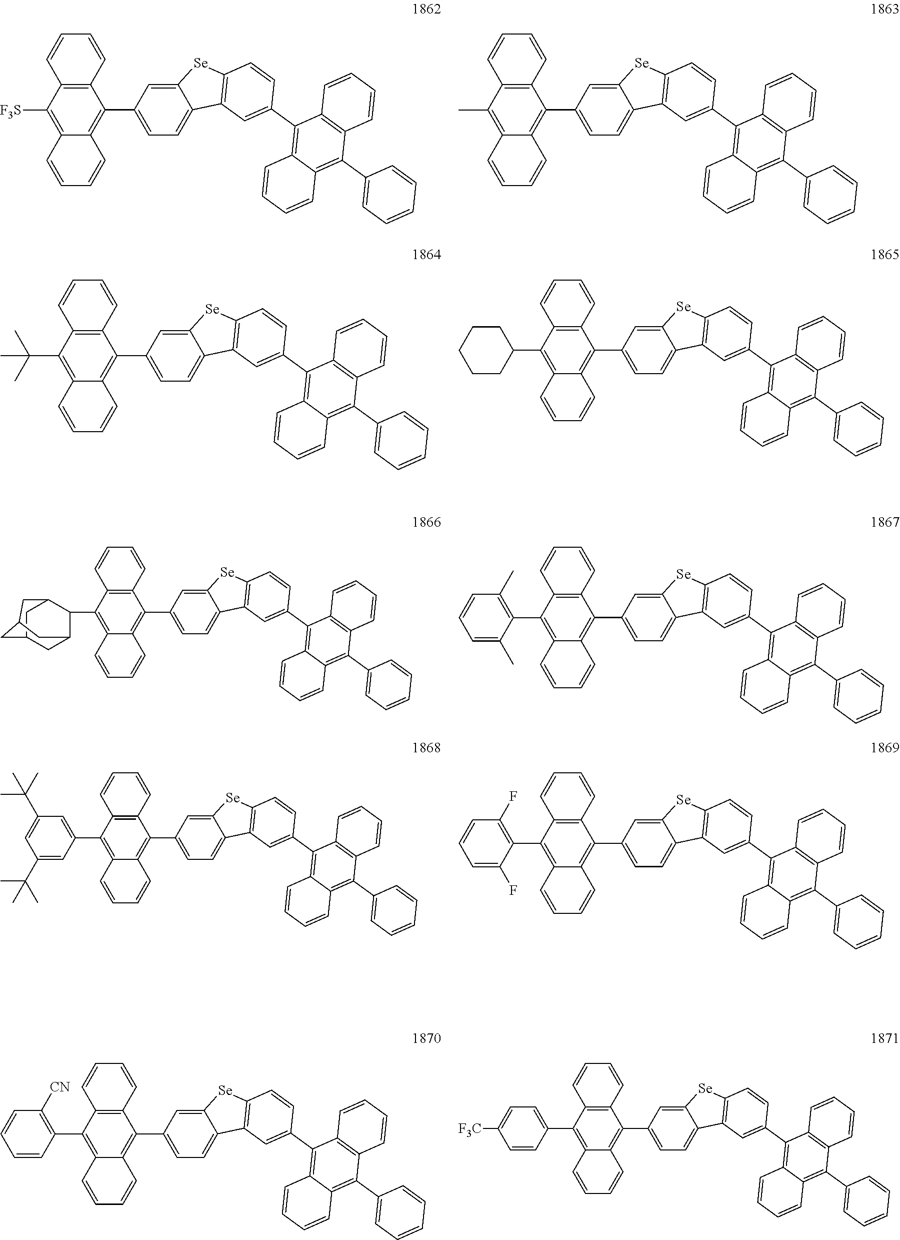

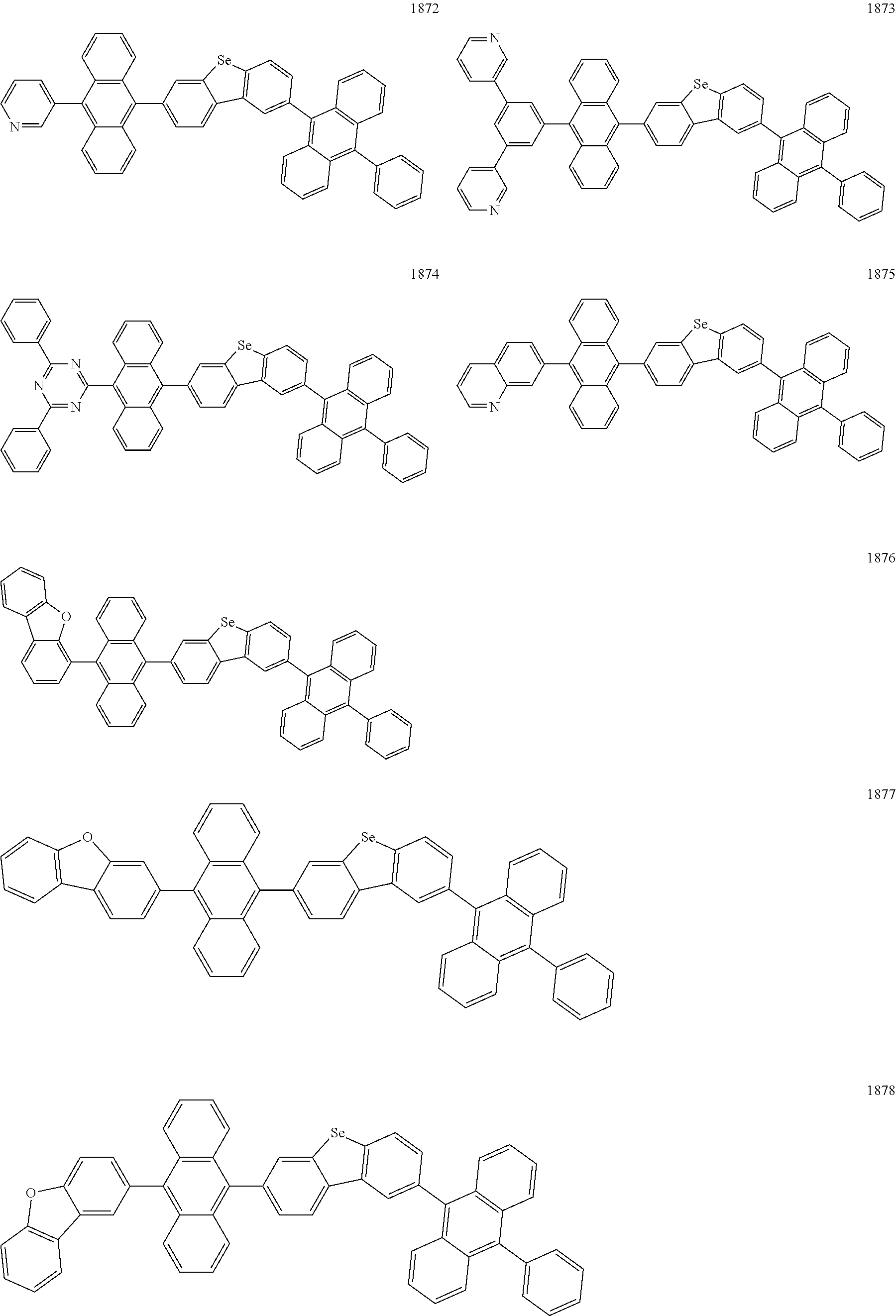

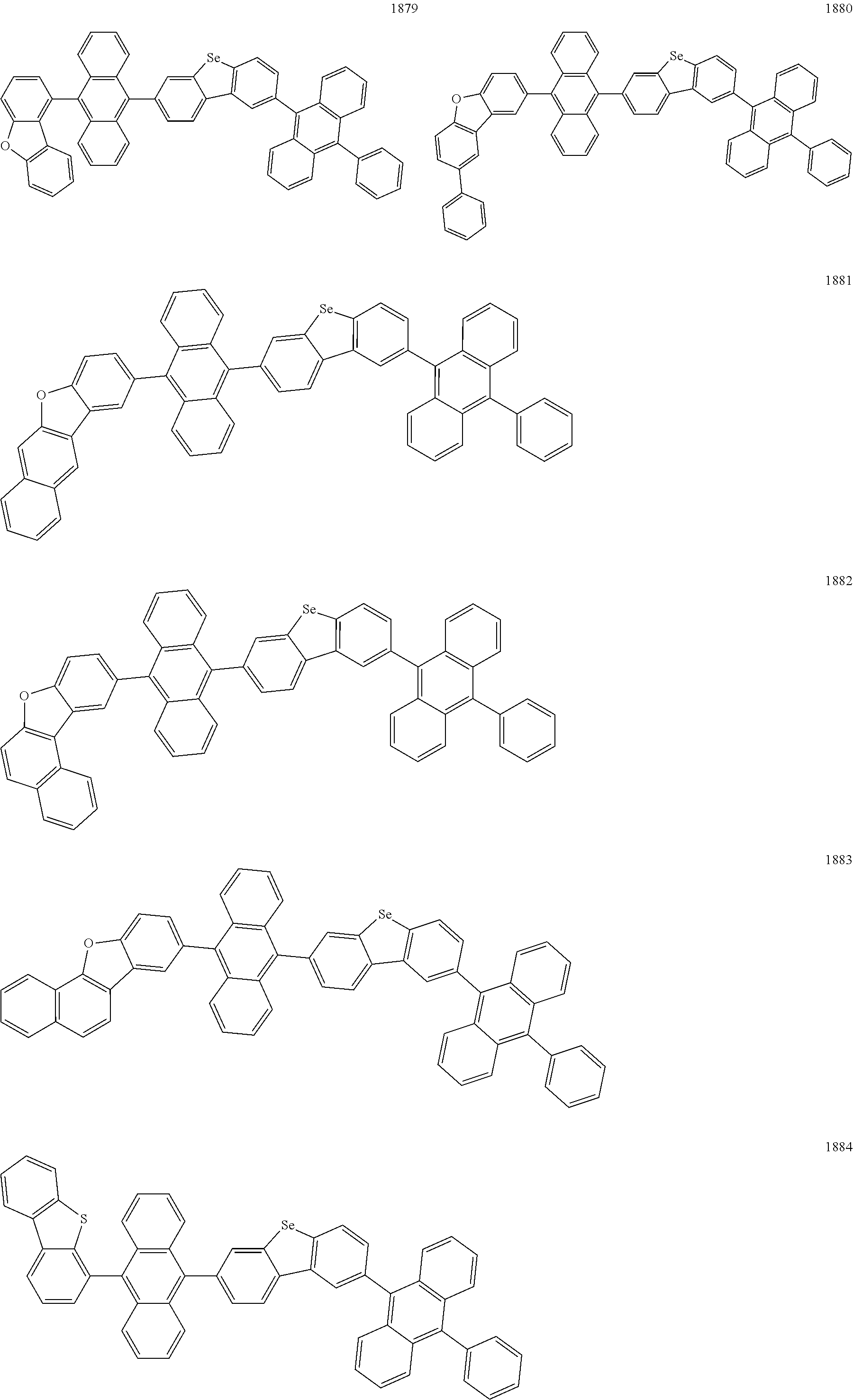

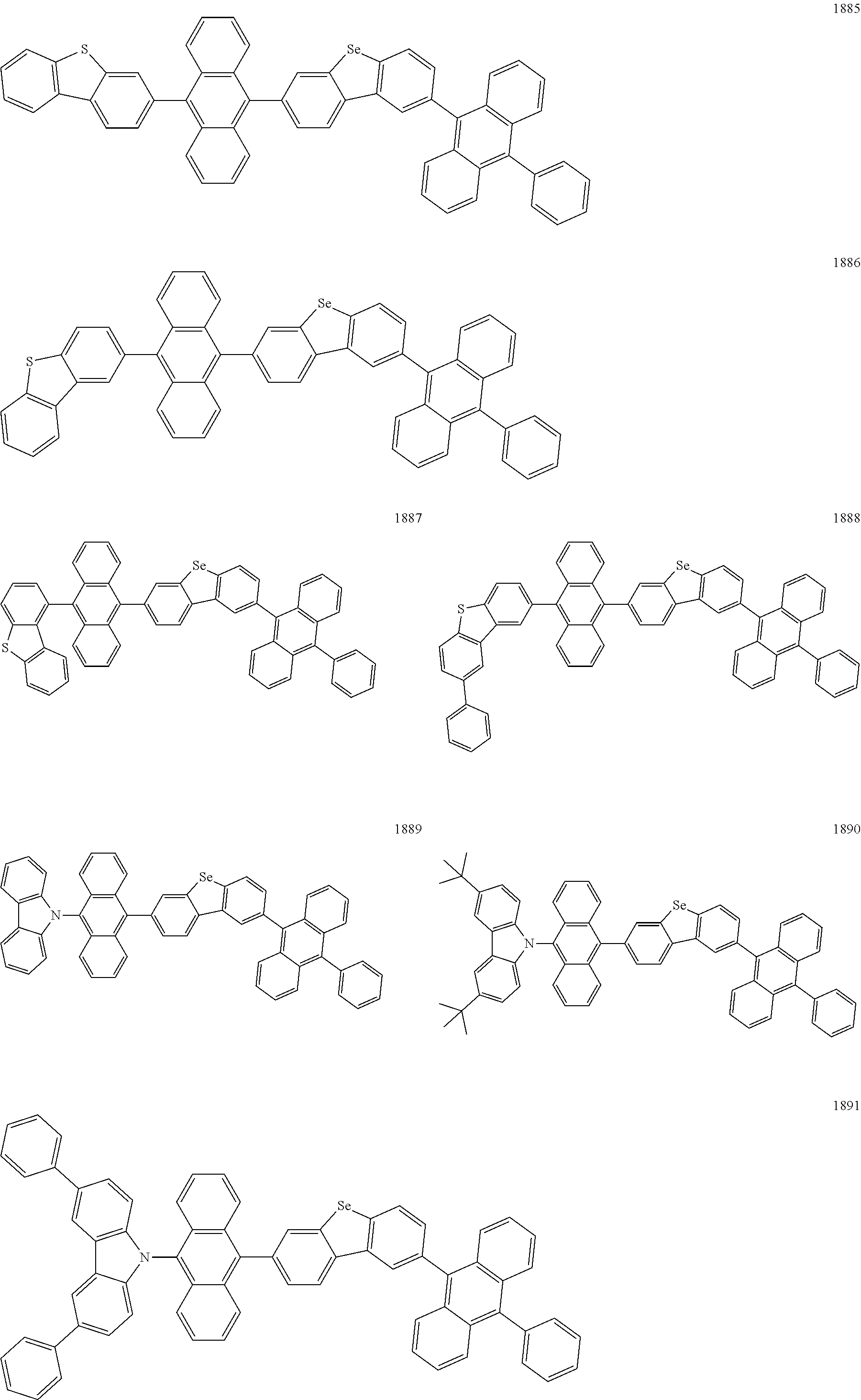

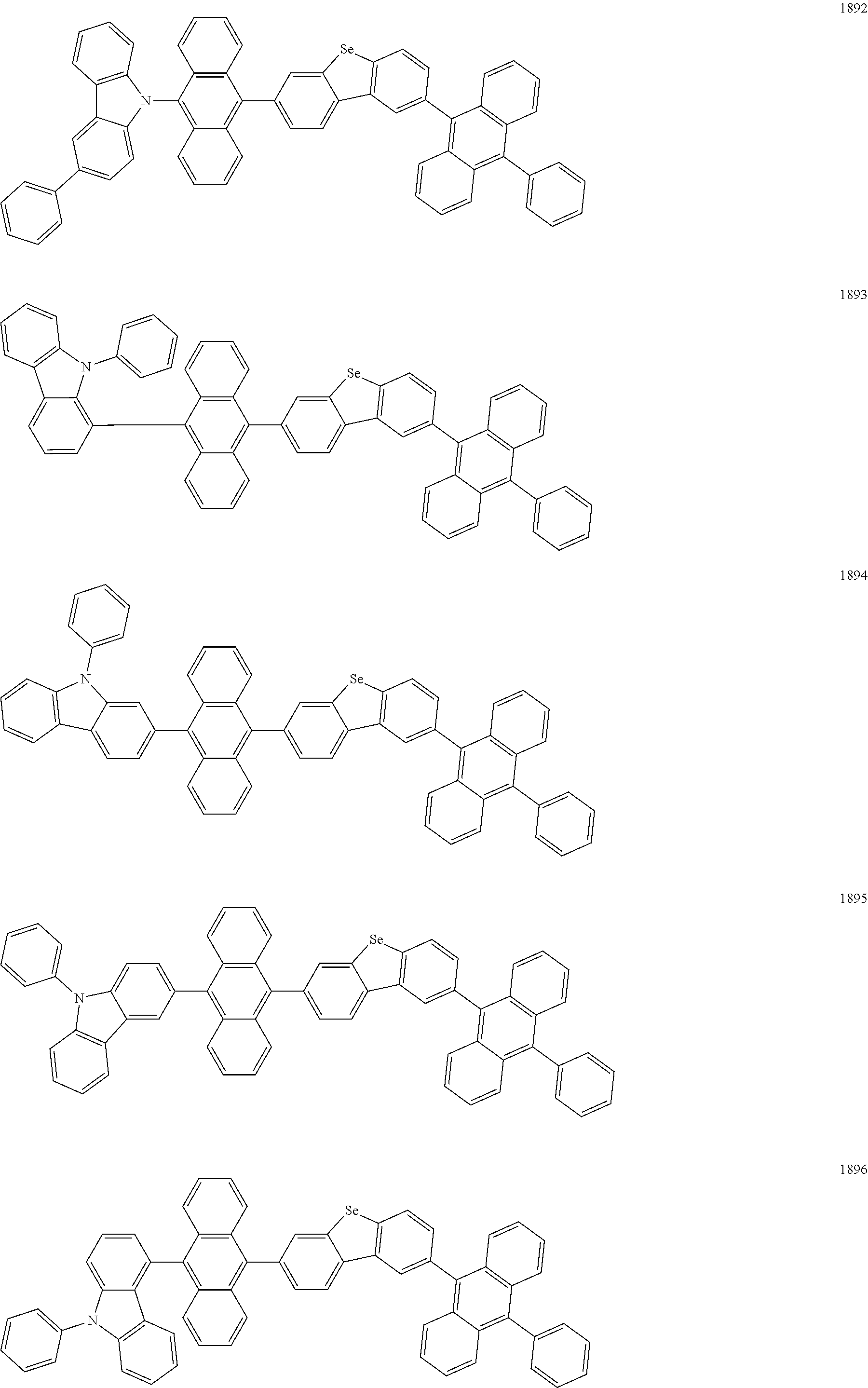

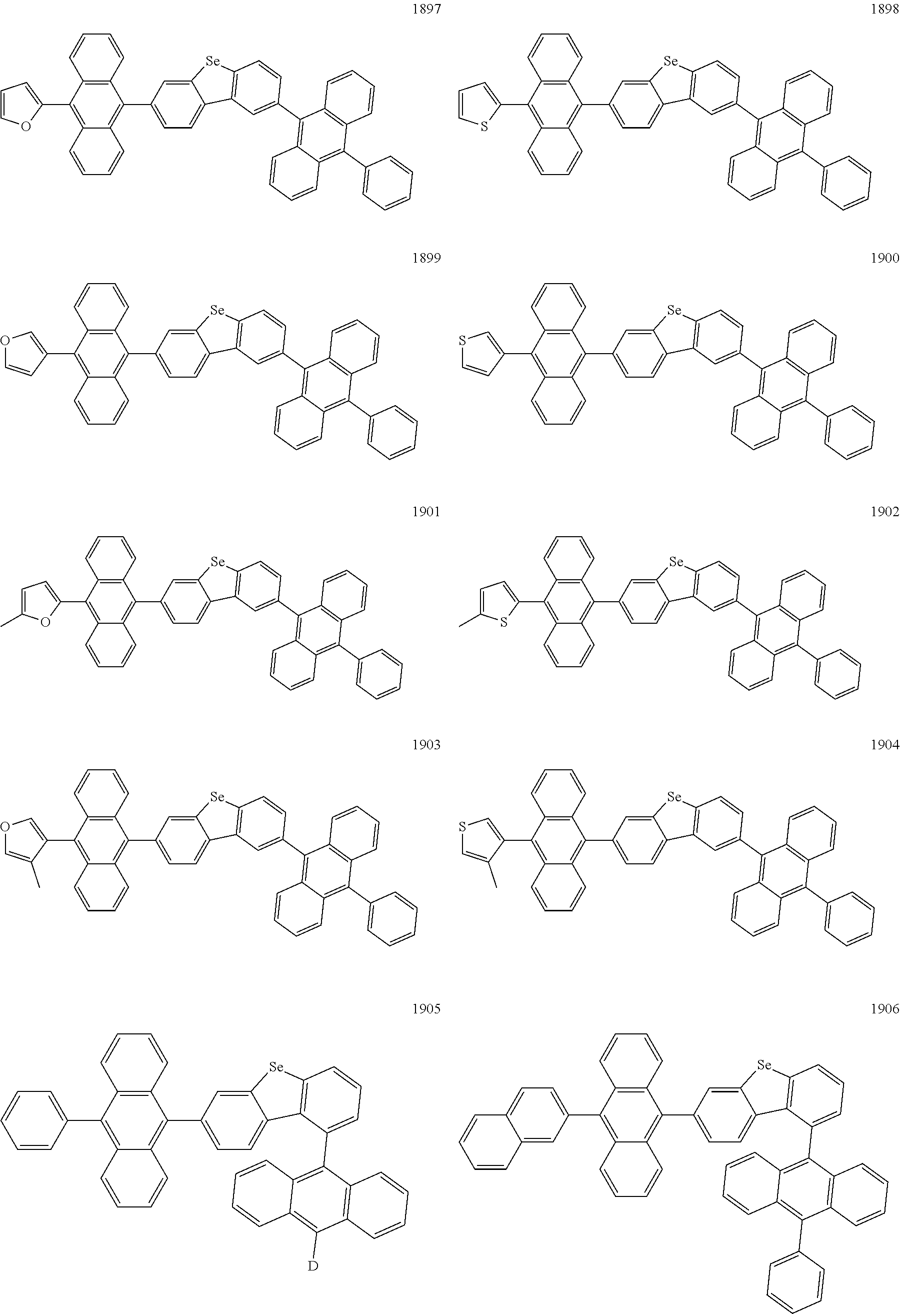

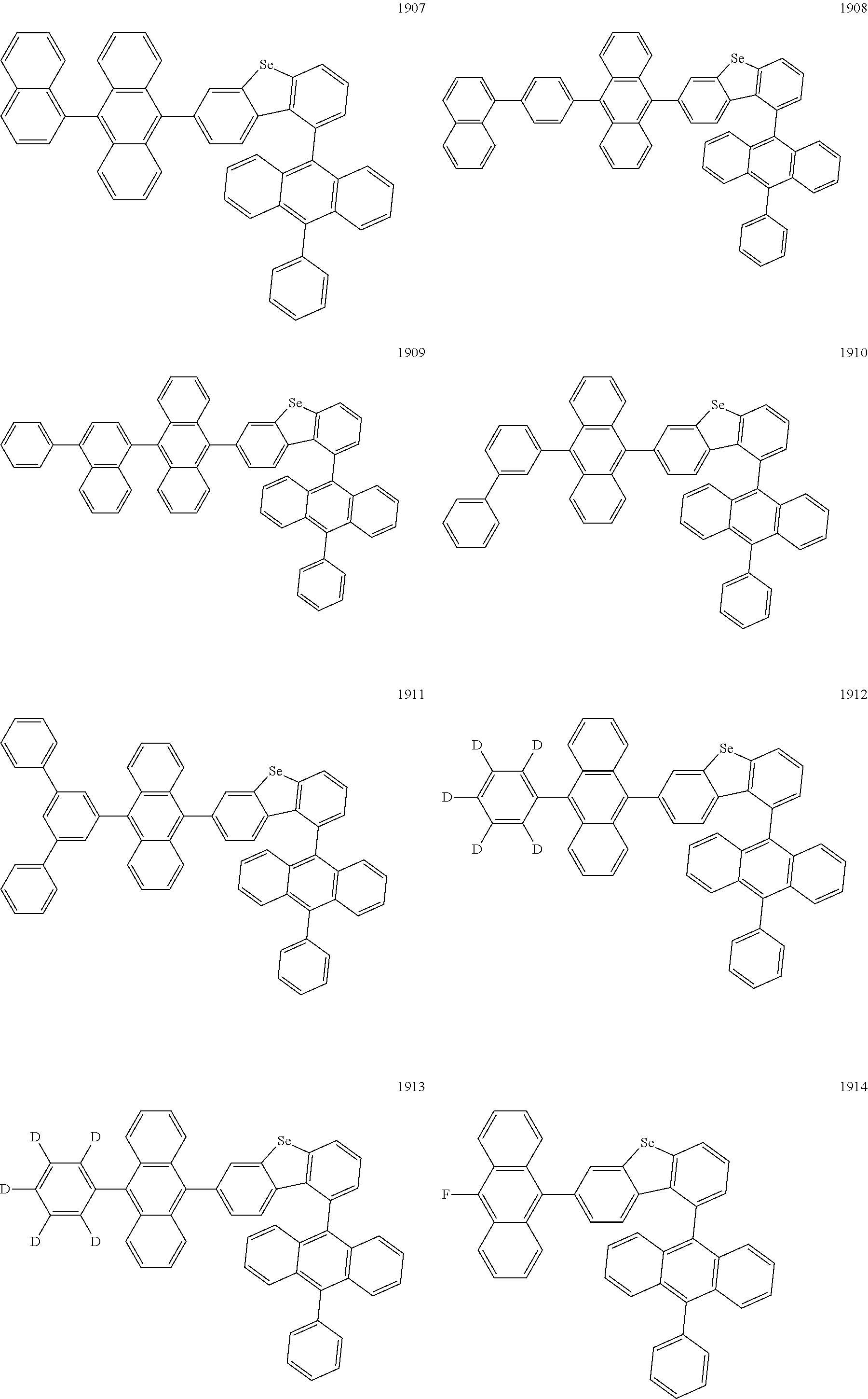

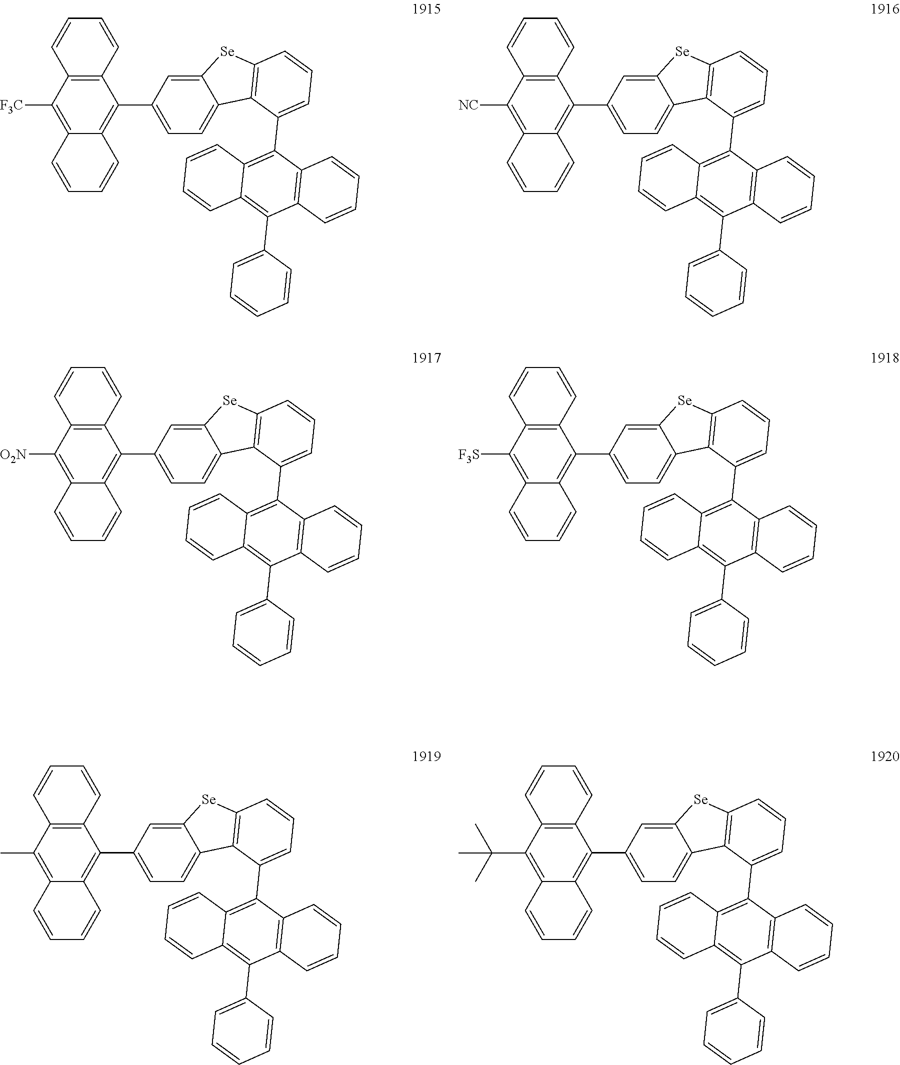

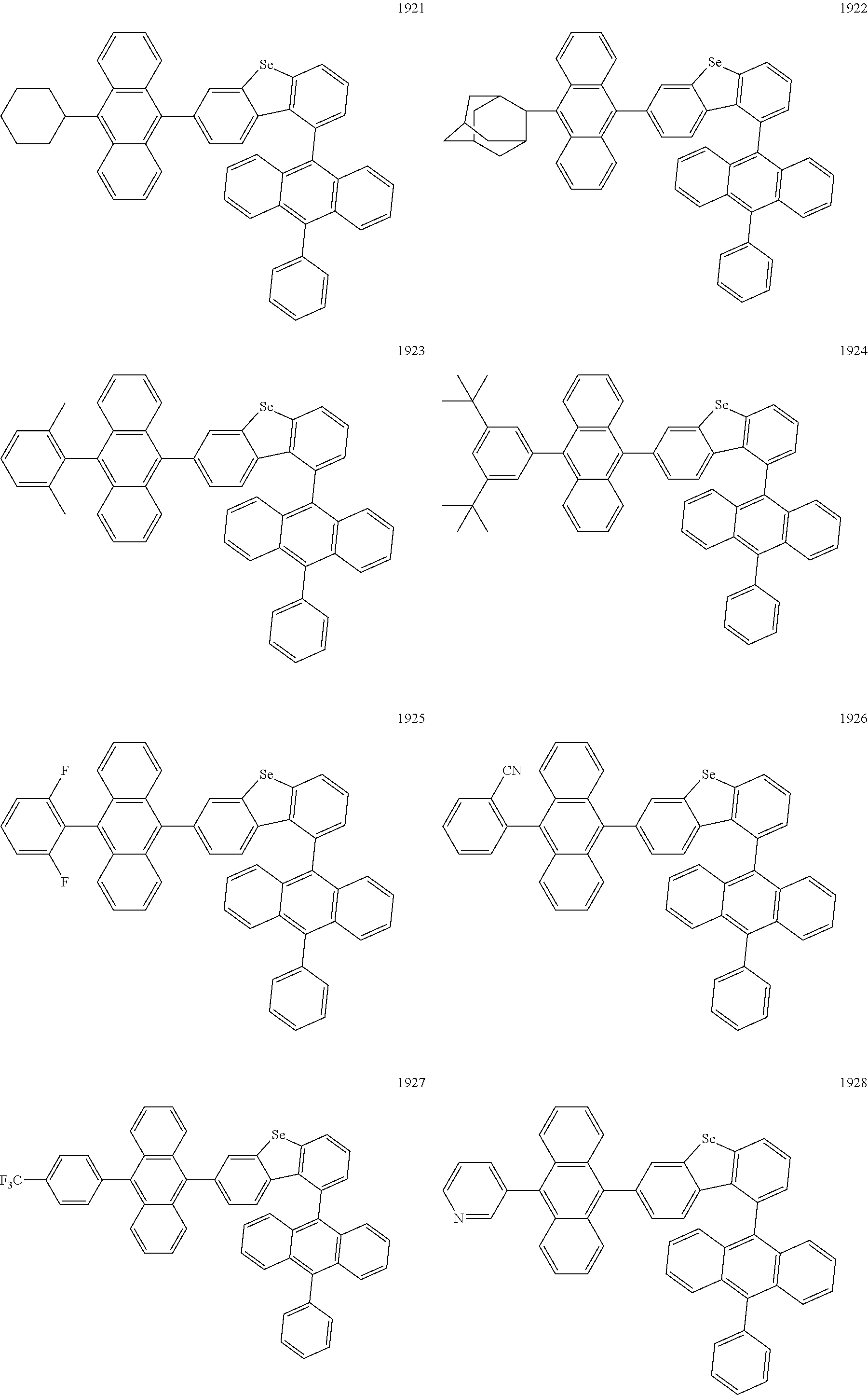

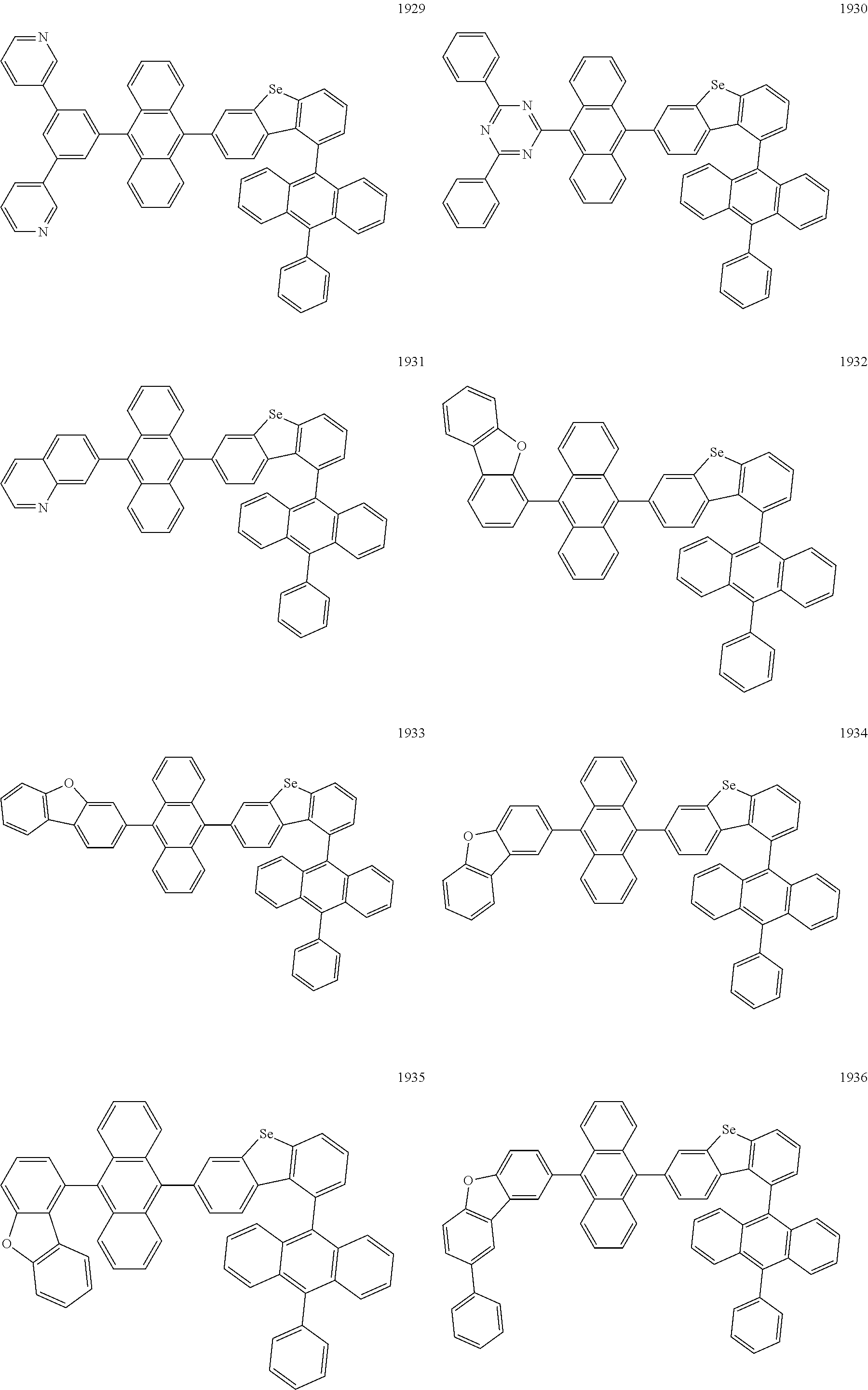

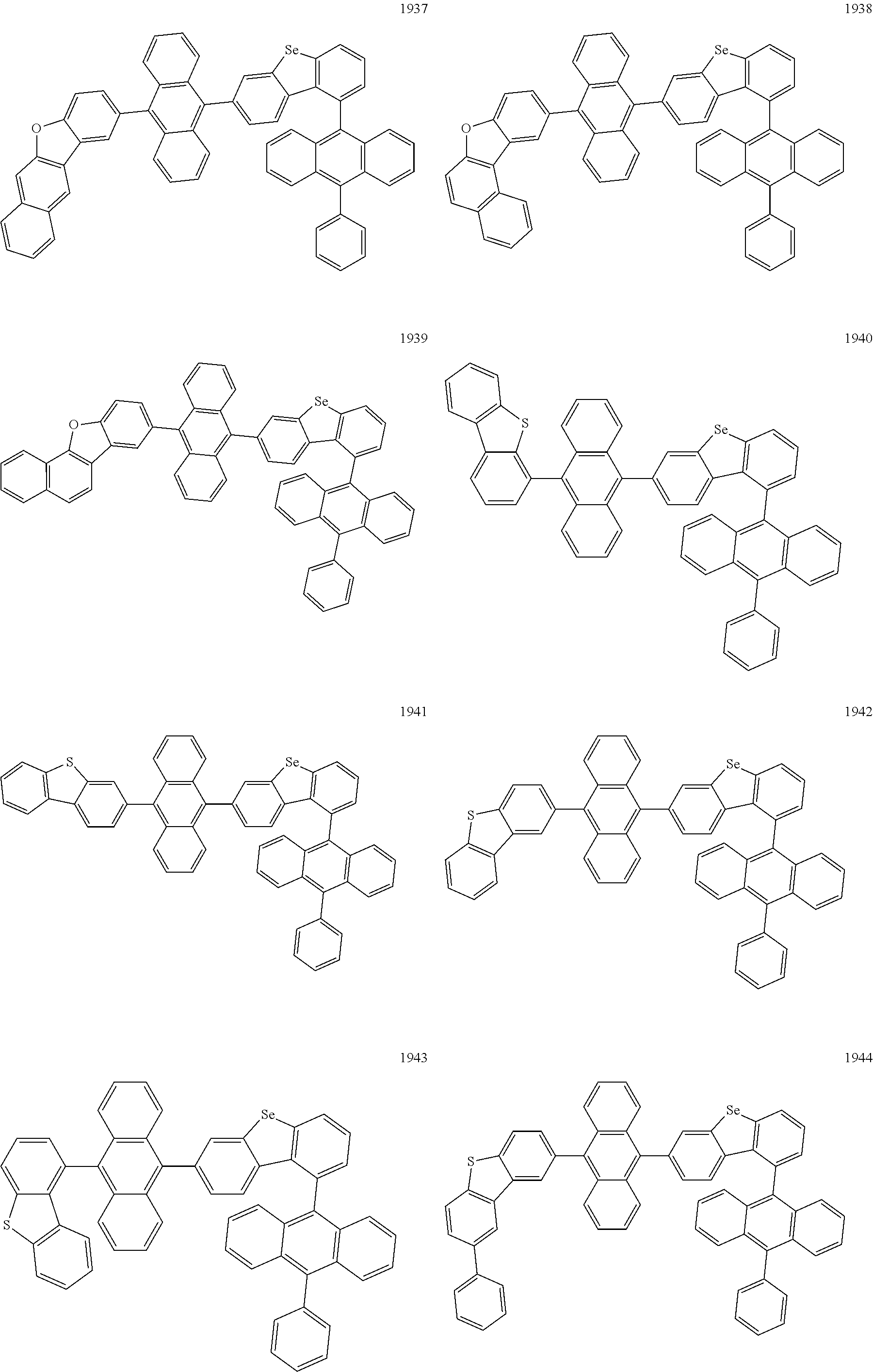

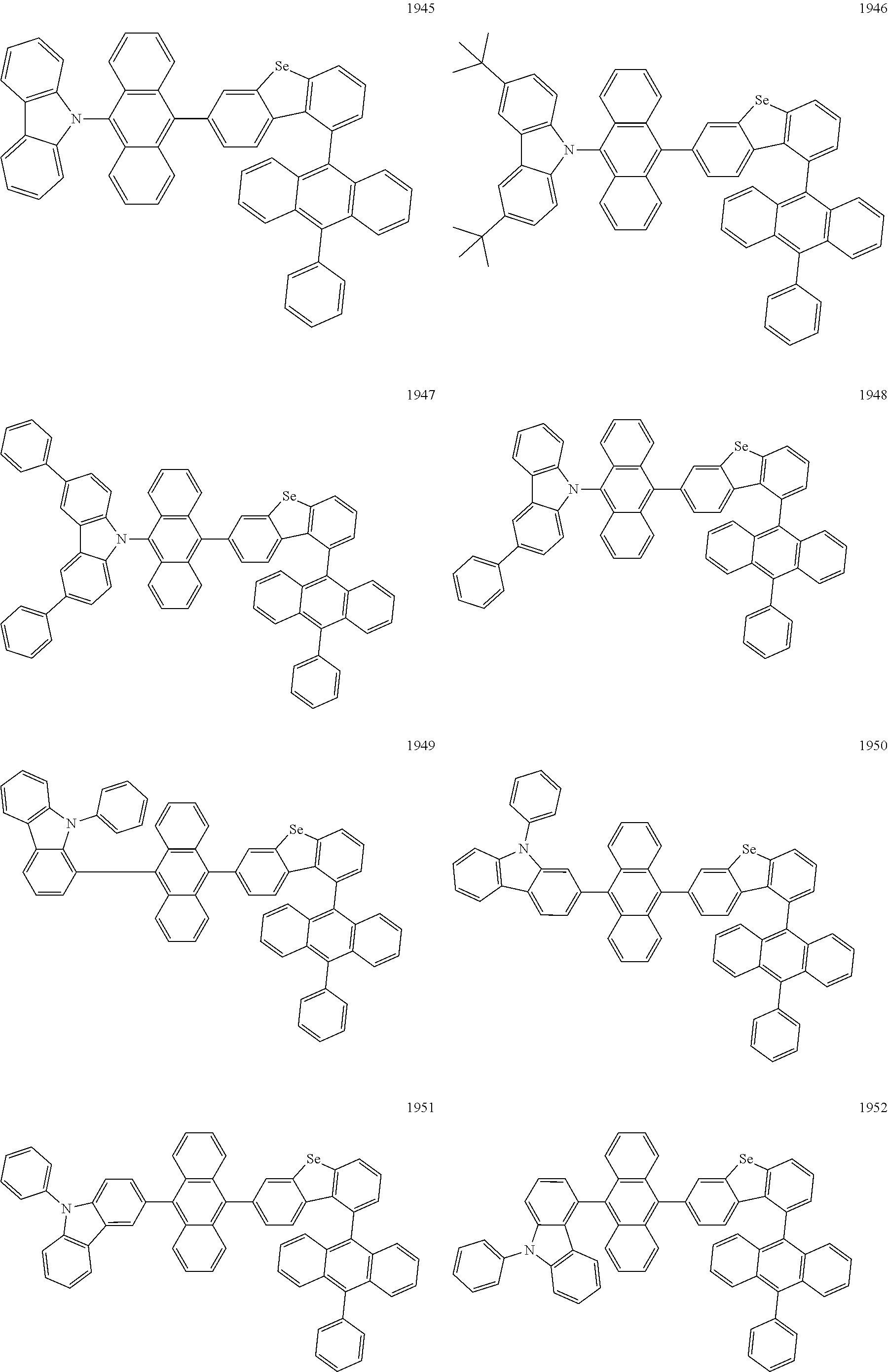

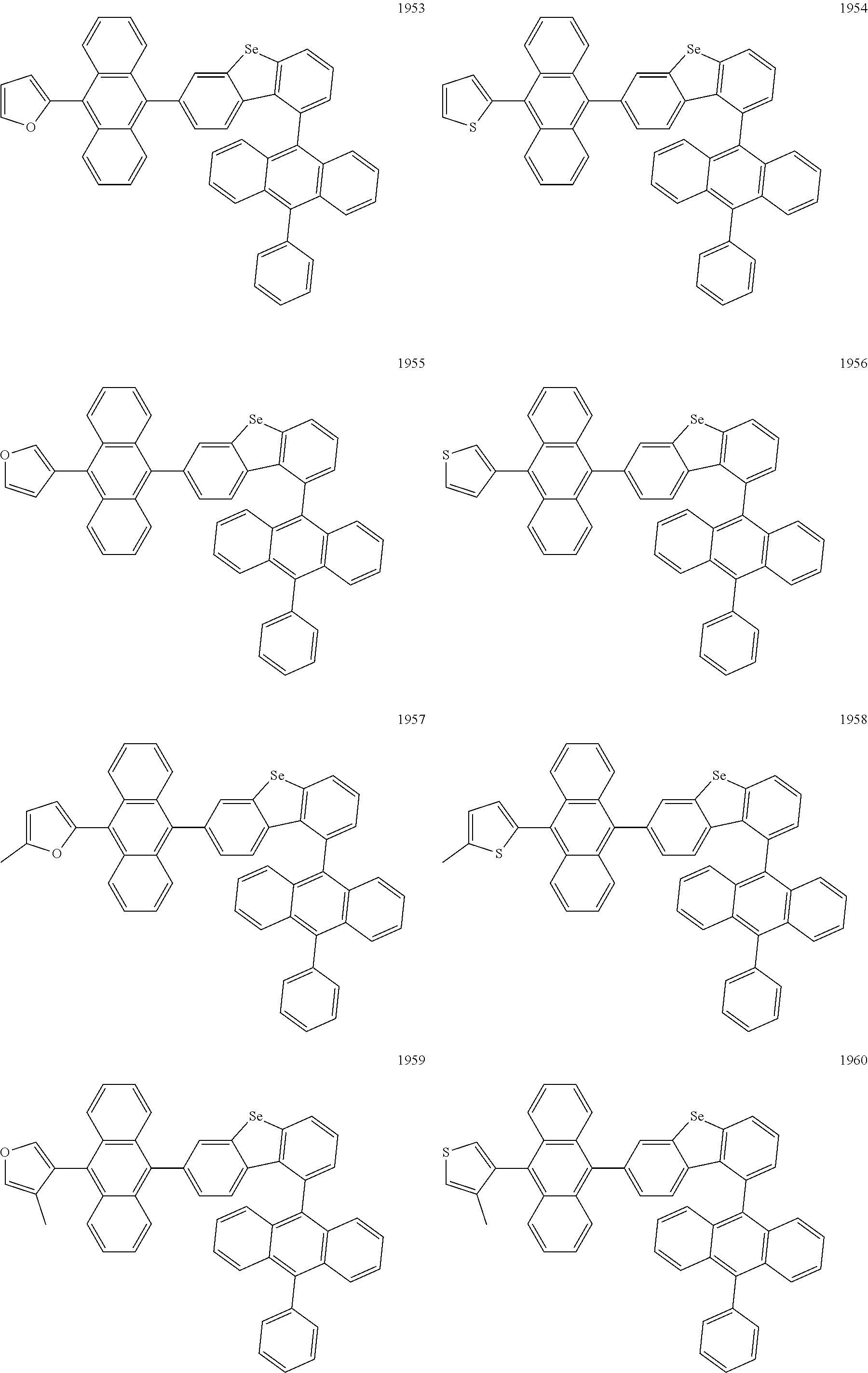

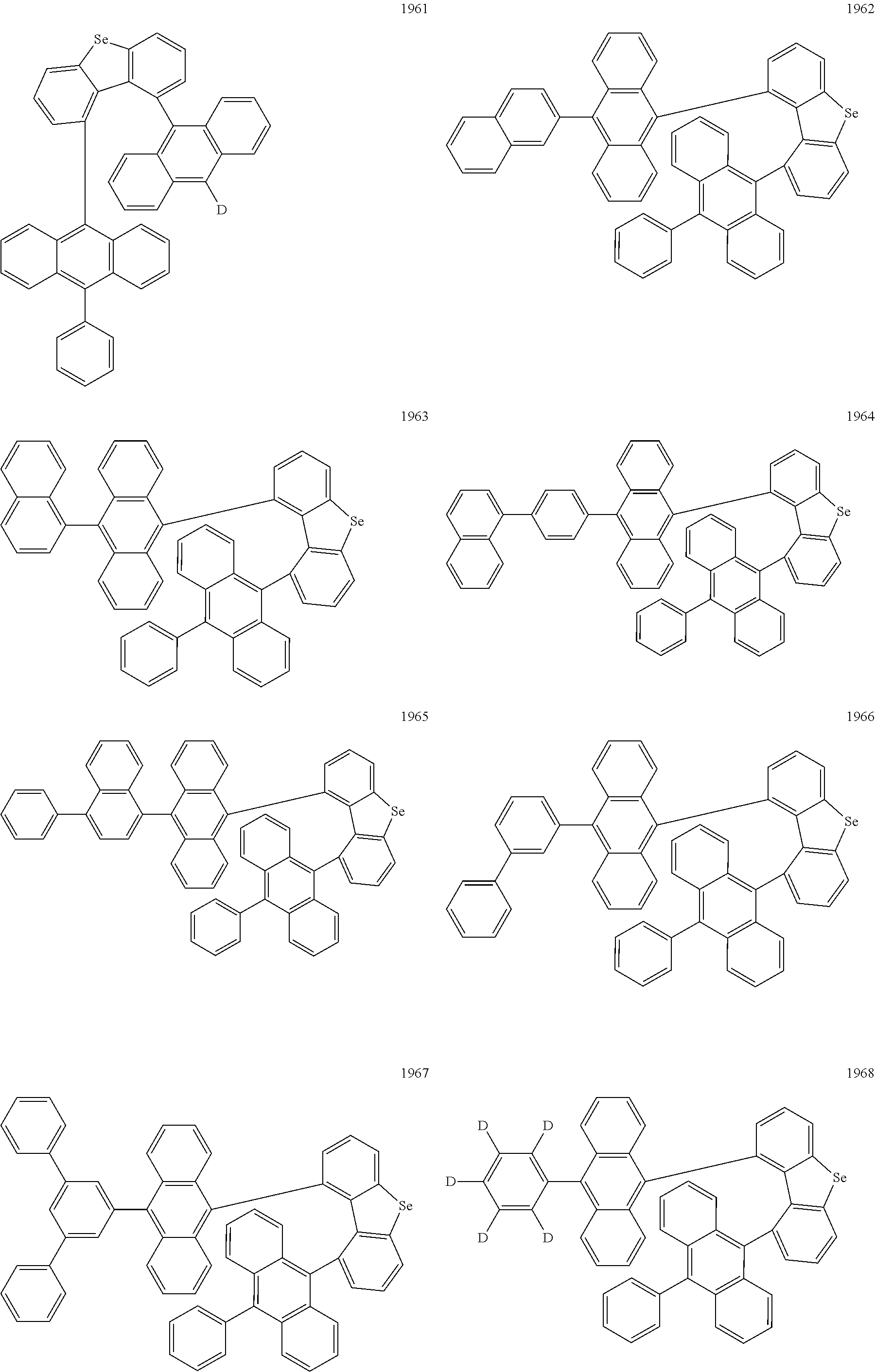

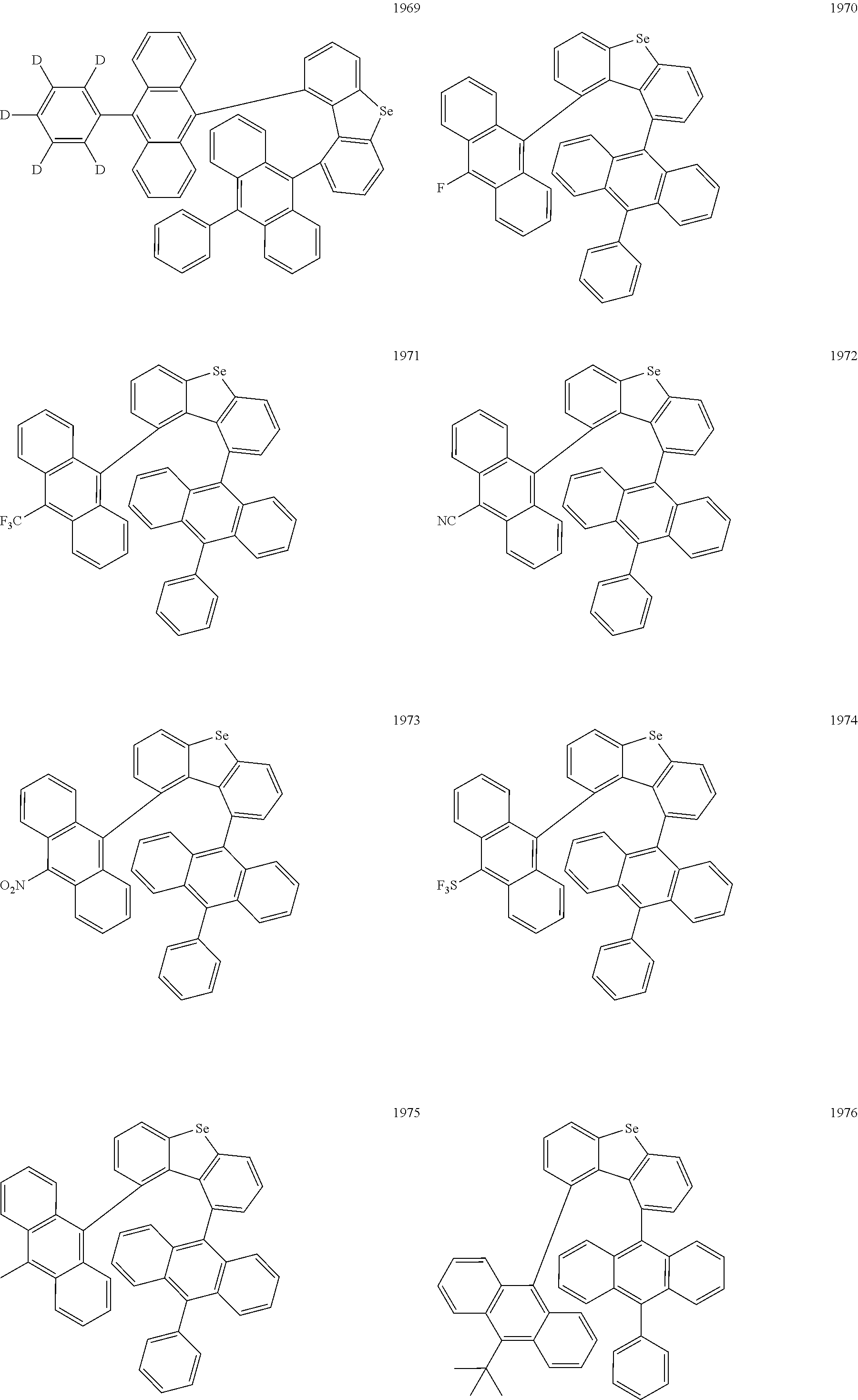

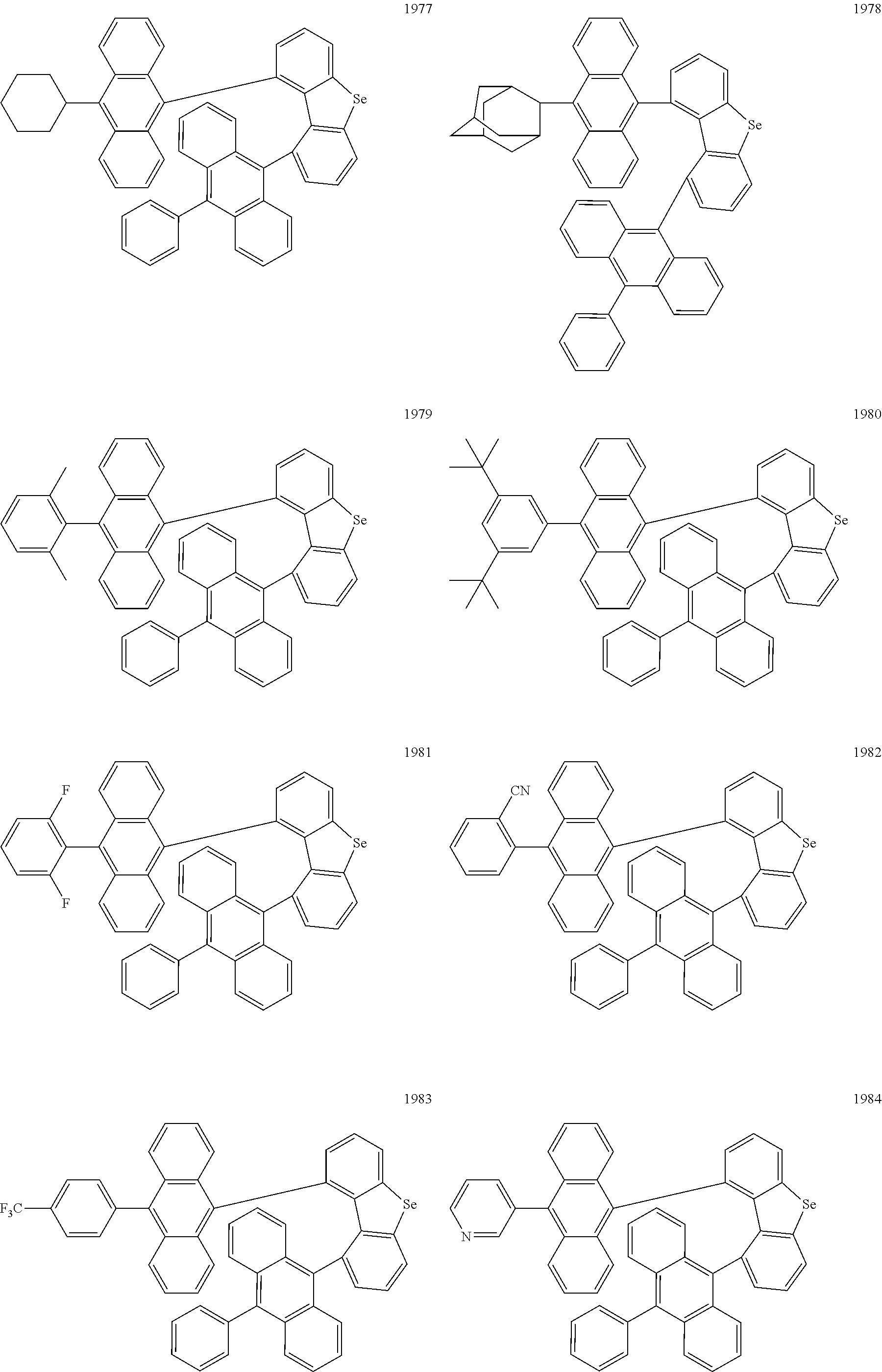

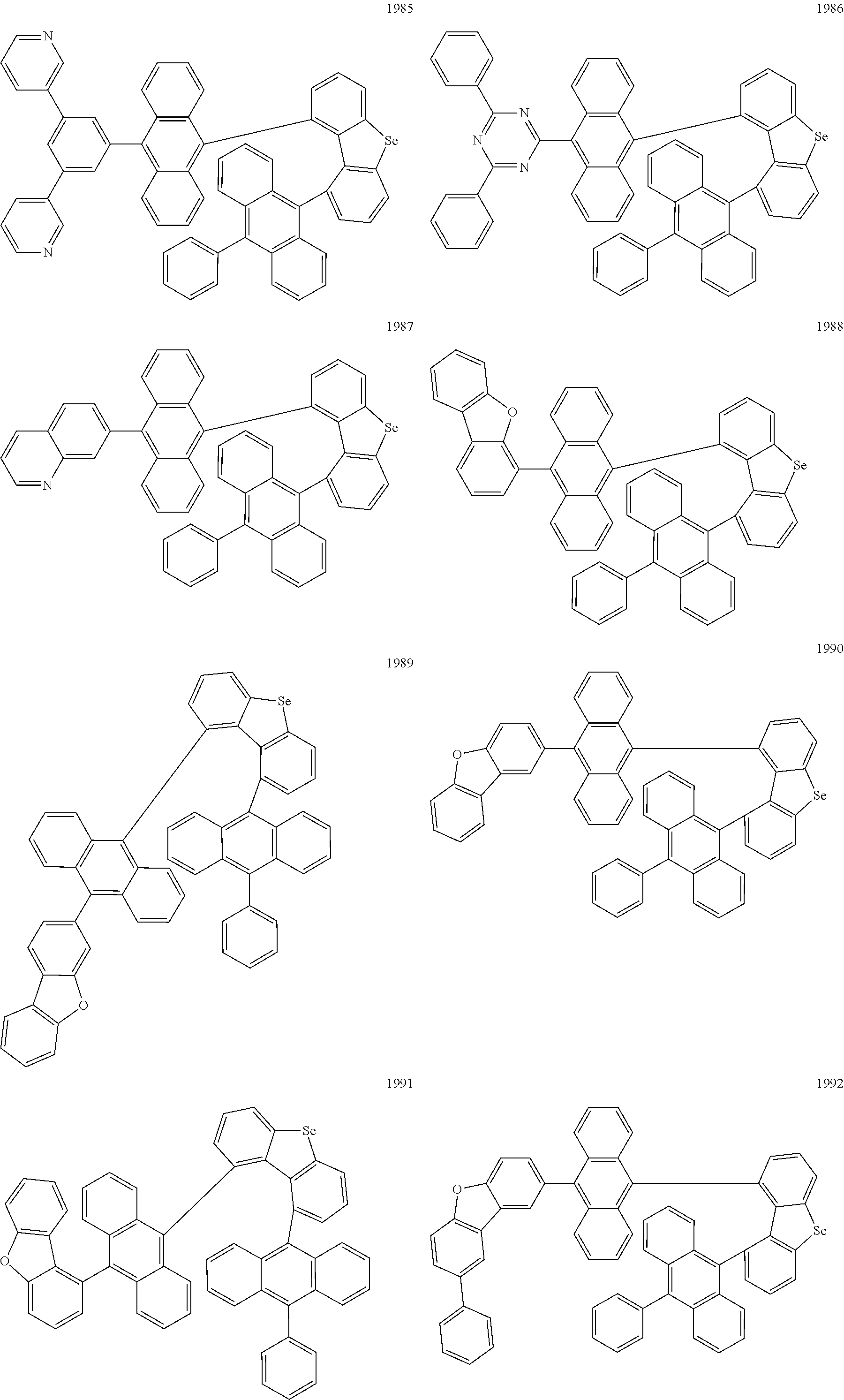

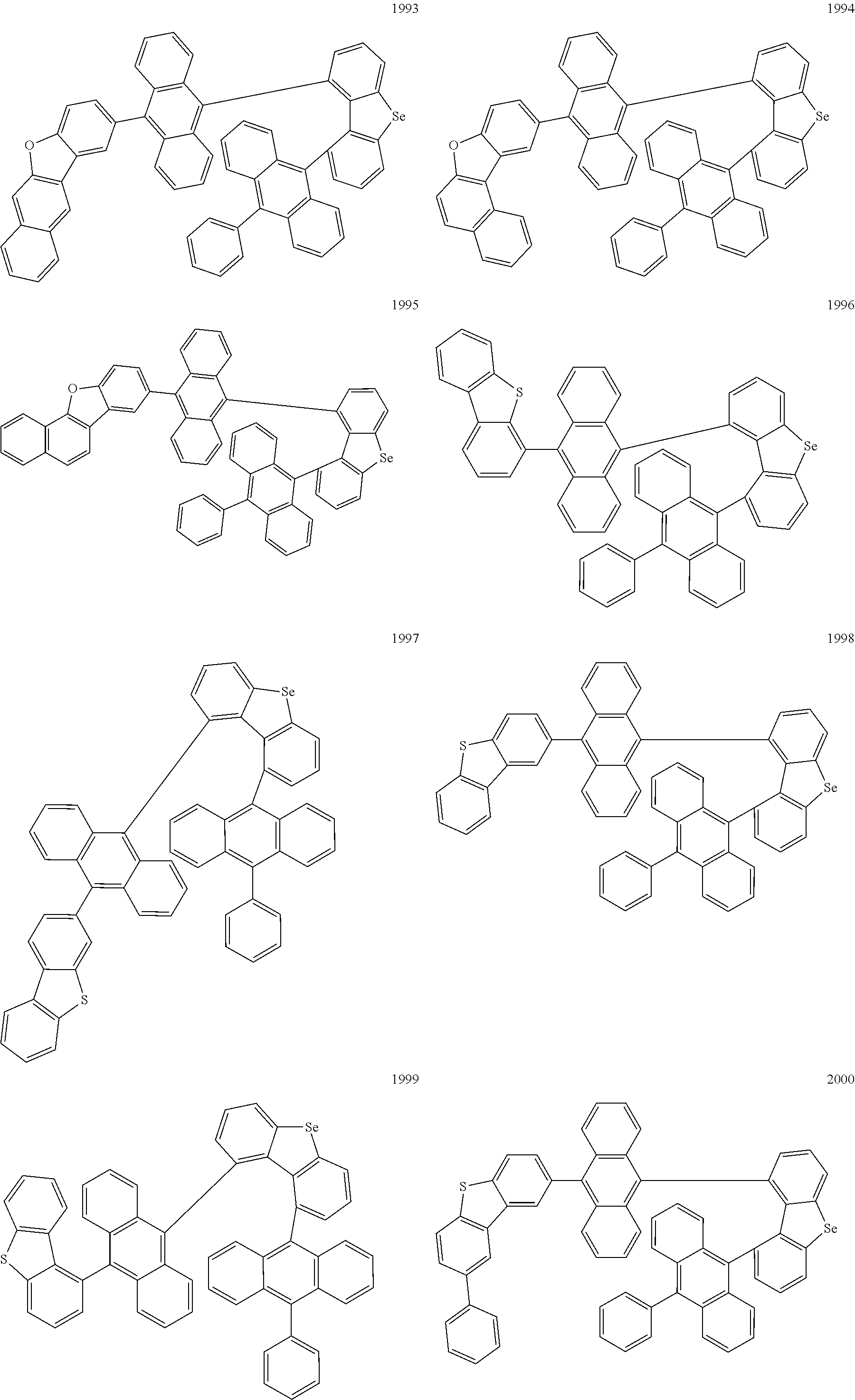

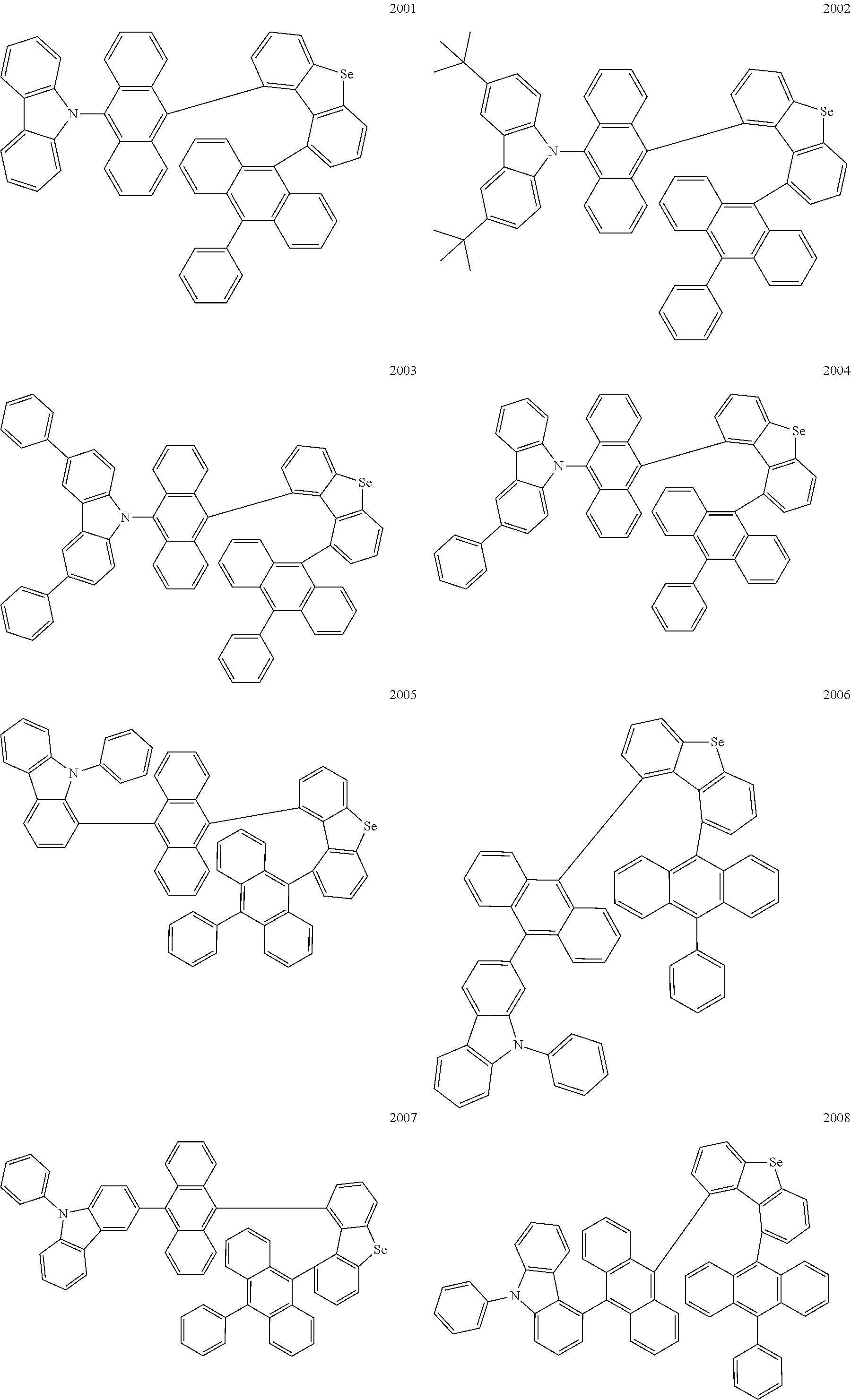

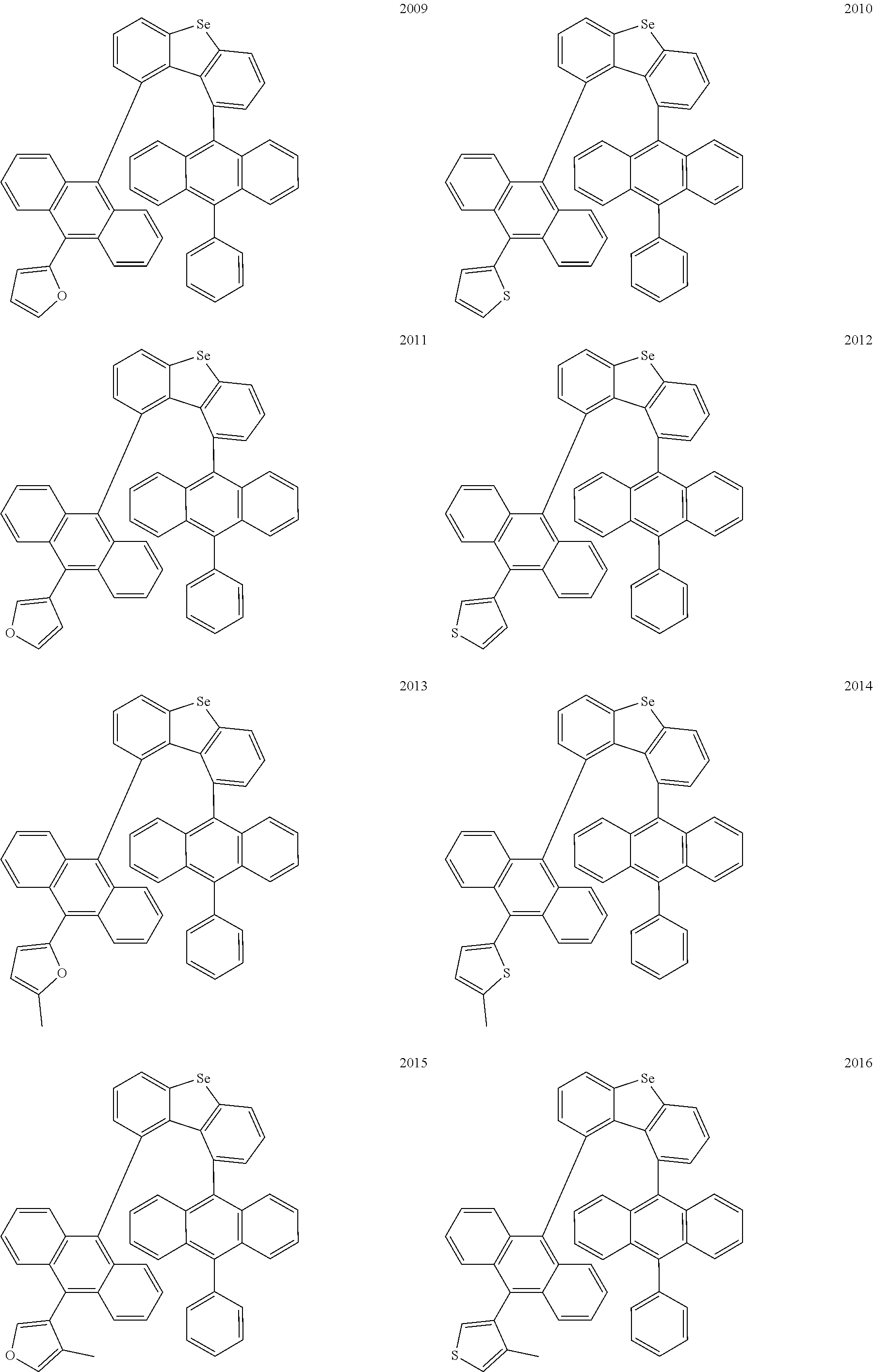

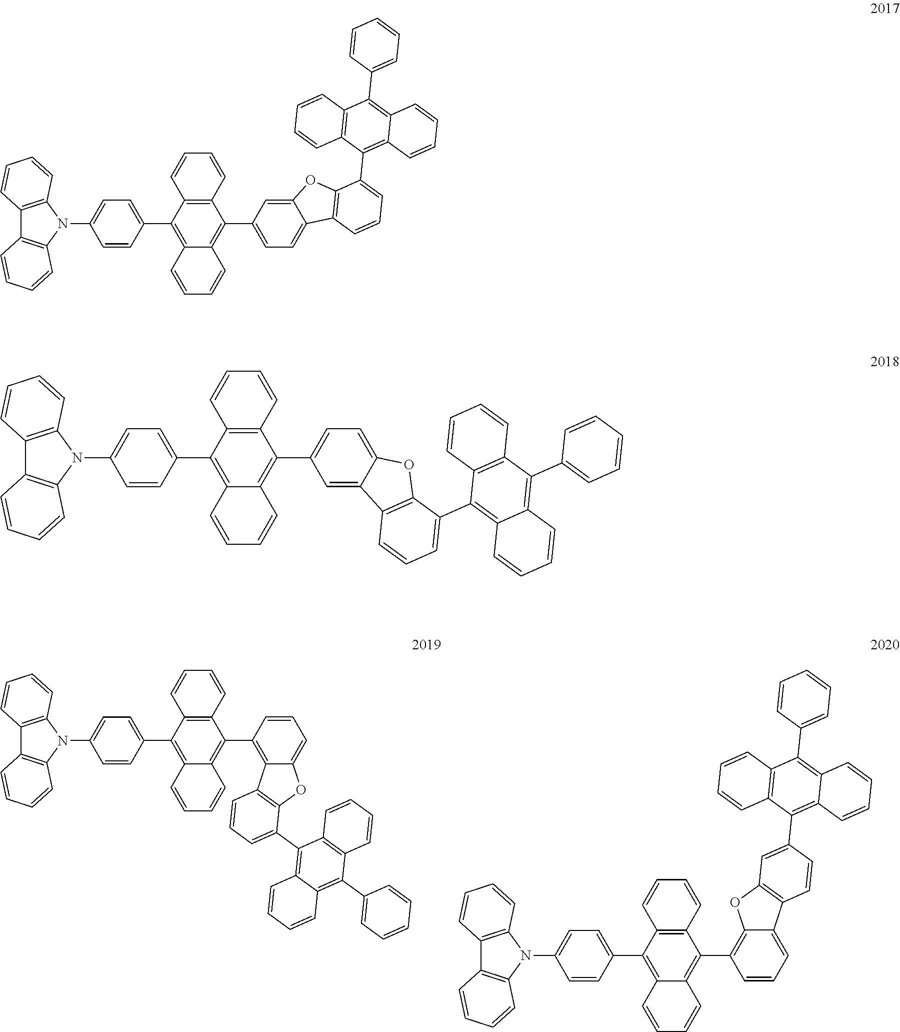 [SMILES: c1ccc(-c2c3ccccc3c(-c3cccc4c3oc3cc(-c5c6ccccc6c(-c6ccc(-n7c8ccccc8c8ccccc87)cc6)c6ccccc56)ccc34)c3ccccc23)cc1 ; c1ccc(-c2c3ccccc3c(-c3cccc4c3oc3ccc(-c5c6ccccc6c(-c6ccc(-n7c8ccccc8c8ccccc87)cc6)c6ccccc56)cc34)c3ccccc23)cc1 ; c1ccc(-c2c3ccccc3c(-c3cccc4c3oc3cccc(-c5c6ccccc6c(-c6ccc(-n7c8ccccc8c8ccccc87)cc6)c6ccccc56)c34)c3ccccc23)cc1 ; c1ccc(-c2c3ccccc3c(-c3ccc4c(c3)oc3c(-c5c6ccccc6c(-c6ccc(-n7c8ccccc8c8ccccc87)cc6)c6ccccc56)cccc34)c3ccccc23)cc1]

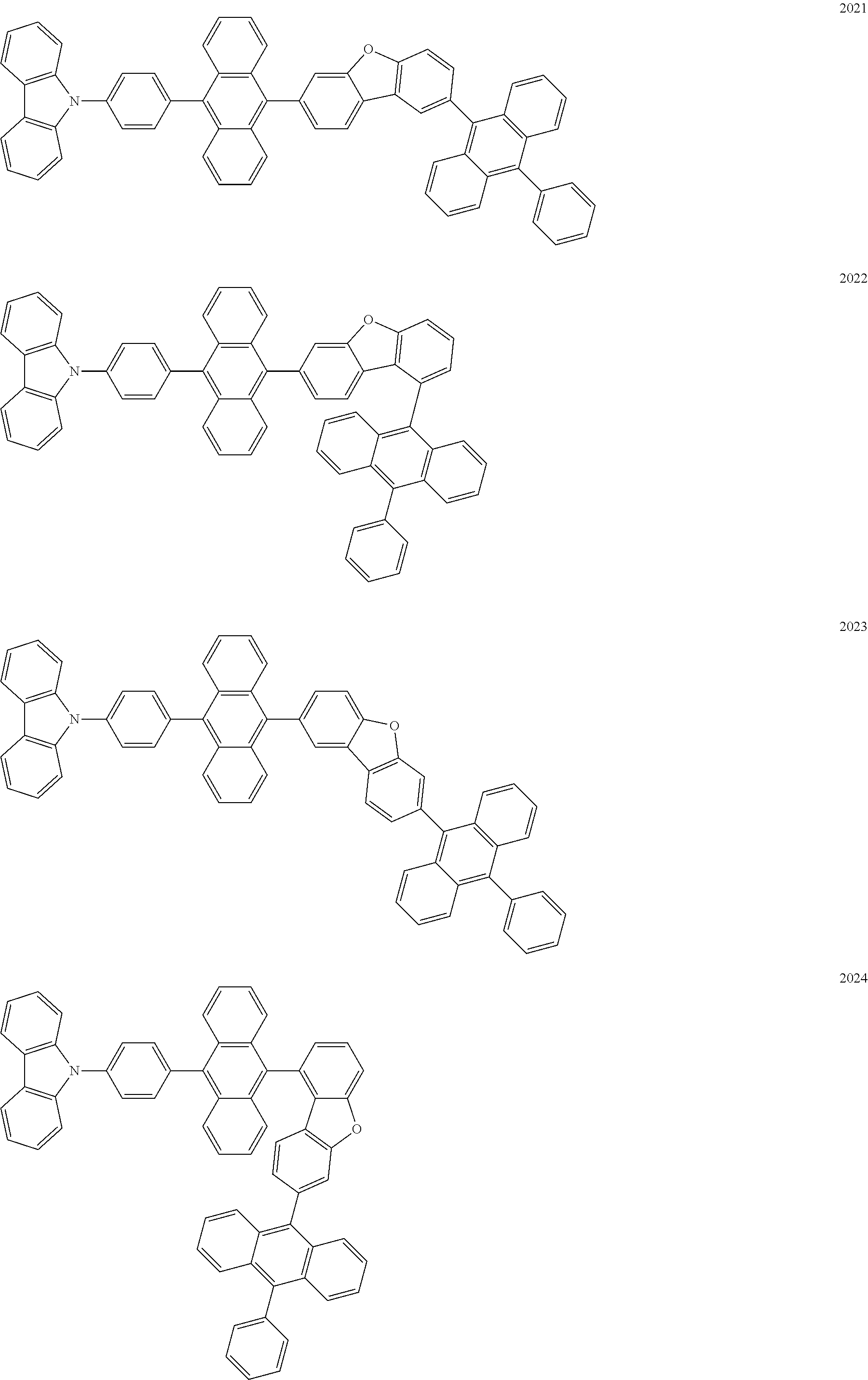 [SMILES: c1ccc(-c2c3ccccc3c(-c3ccc4oc5cc(-c6c7ccccc7c(-c7ccc(-n8c9ccccc9c9ccccc98)cc7)c7ccccc67)ccc5c4c3)c3ccccc23)cc1 ; c1ccc(-c2c3ccccc3c(-c3cccc4oc5cc(-c6c7ccccc7c(-c7ccc(-n8c9ccccc9c9ccccc98)cc7)c7ccccc67)ccc5c34)c3ccccc23)cc1 ; c1ccc(-c2c3ccccc3c(-c3ccc4c(c3)oc3ccc(-c5c6ccccc6c(-c6ccc(-n7c8ccccc8c8ccccc87)cc6)c6ccccc56)cc34)c3ccccc23)cc1 ; c1ccc(-c2c3ccccc3c(-c3ccc4c(c3)oc3cccc(-c5c6ccccc6c(-c6ccc(-n7c8ccccc8c8ccccc87)cc6)c6ccccc56)c34)c3ccccc23)cc1]

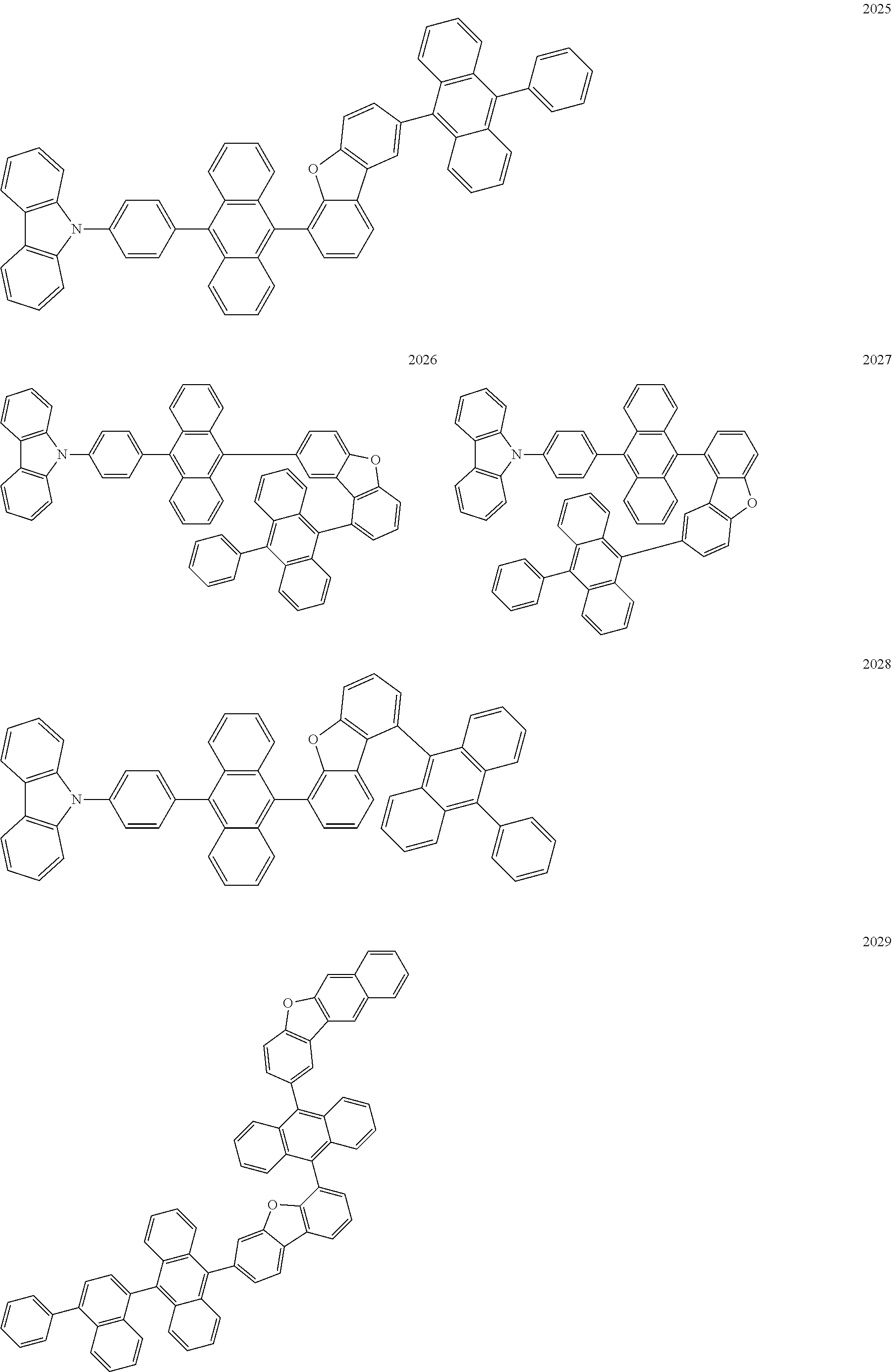

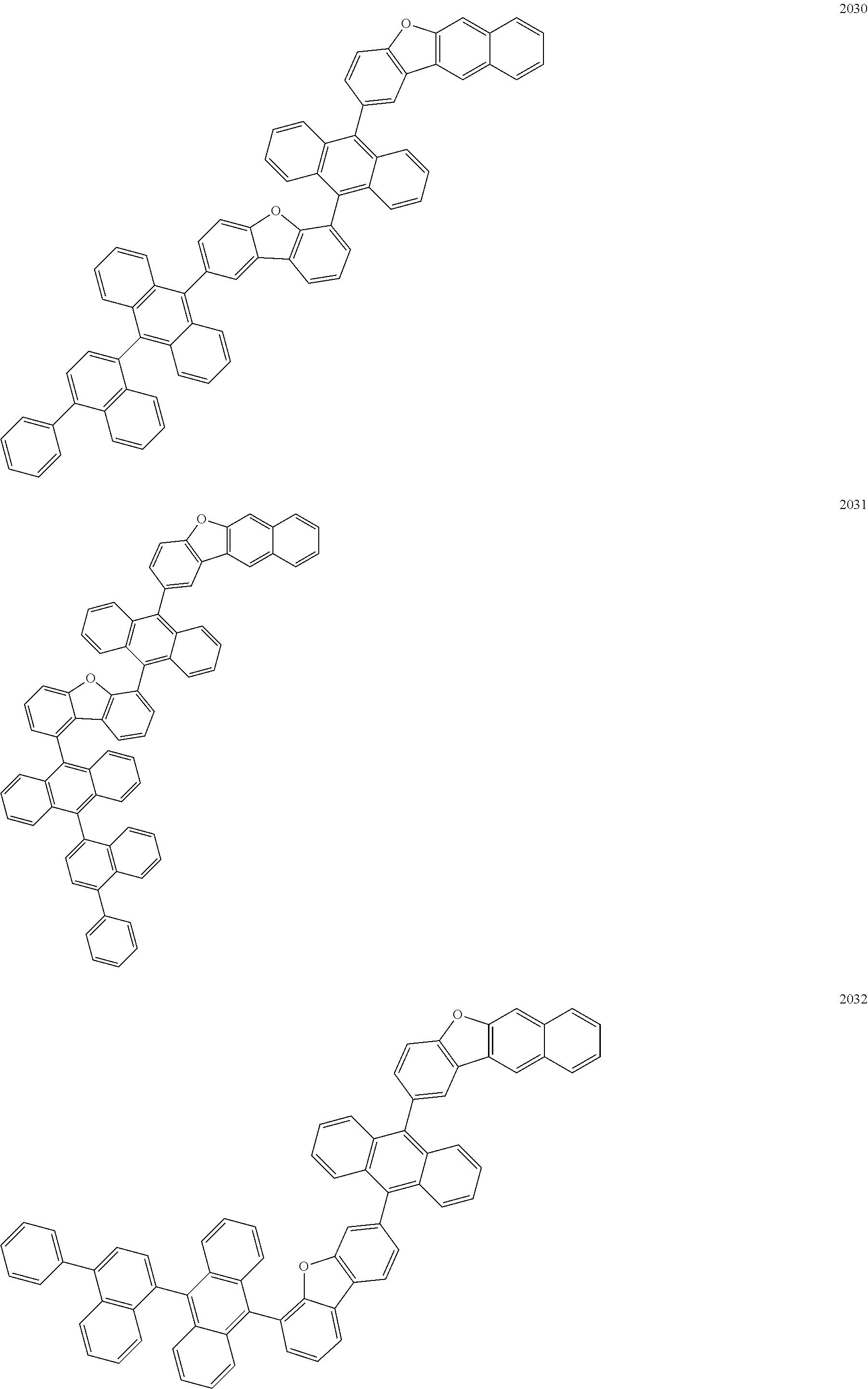 [SMILES: c1ccc(-c2ccc(-c3c4ccccc4c(-c4ccc5oc6c(-c7c8ccccc8c(-c8ccc9oc%10cc%11ccccc%11cc%10c9c8)c8ccccc78)cccc6c5c4)c4ccccc34)c3ccccc23)cc1 ; c1ccc(-c2ccc(-c3c4ccccc4c(-c4cccc5oc6c(-c7c8ccccc8c(-c8ccc9oc%10cc%11ccccc%11cc%10c9c8)c8ccccc78)cccc6c45)c4ccccc34)c3ccccc23)cc1 ; c1ccc(-c2ccc(-c3c4ccccc4c(-c4cccc5c4oc4cc(-c6c7ccccc7c(-c7ccc8oc9cc%10ccccc%10cc9c8c7)c7ccccc67)ccc45)c4ccccc34)c3ccccc23)cc1]

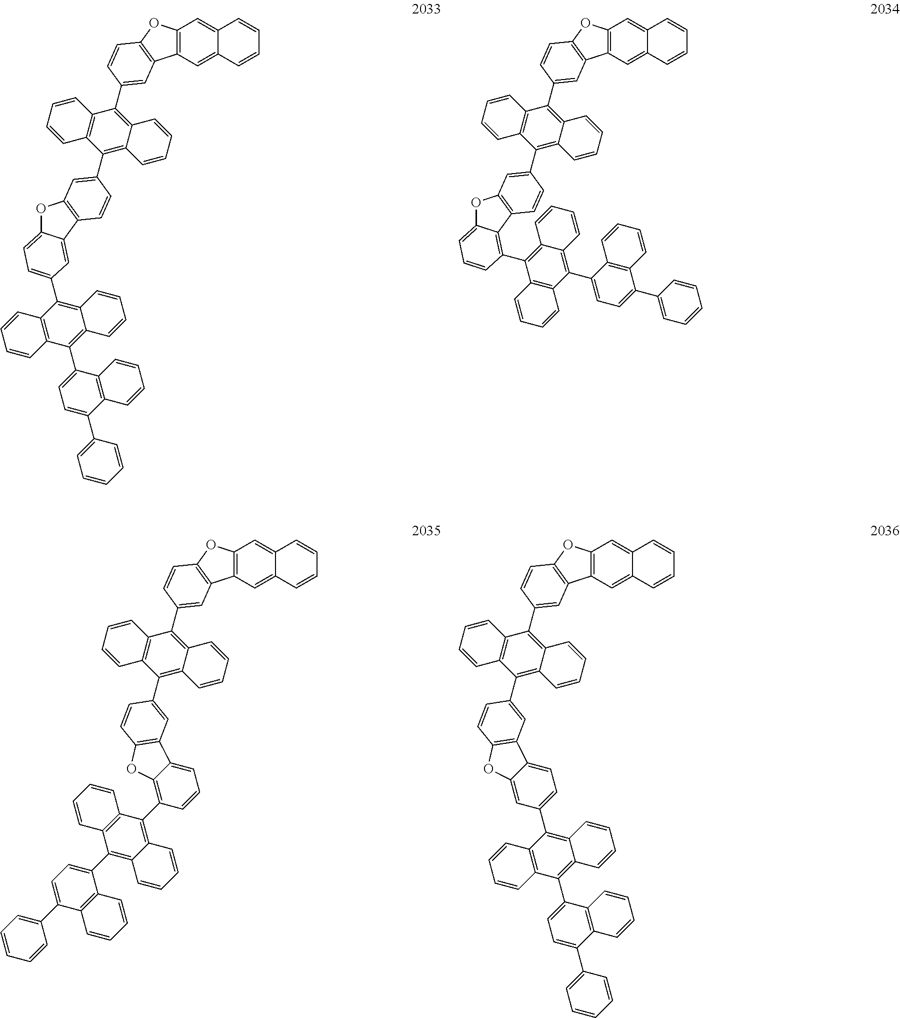

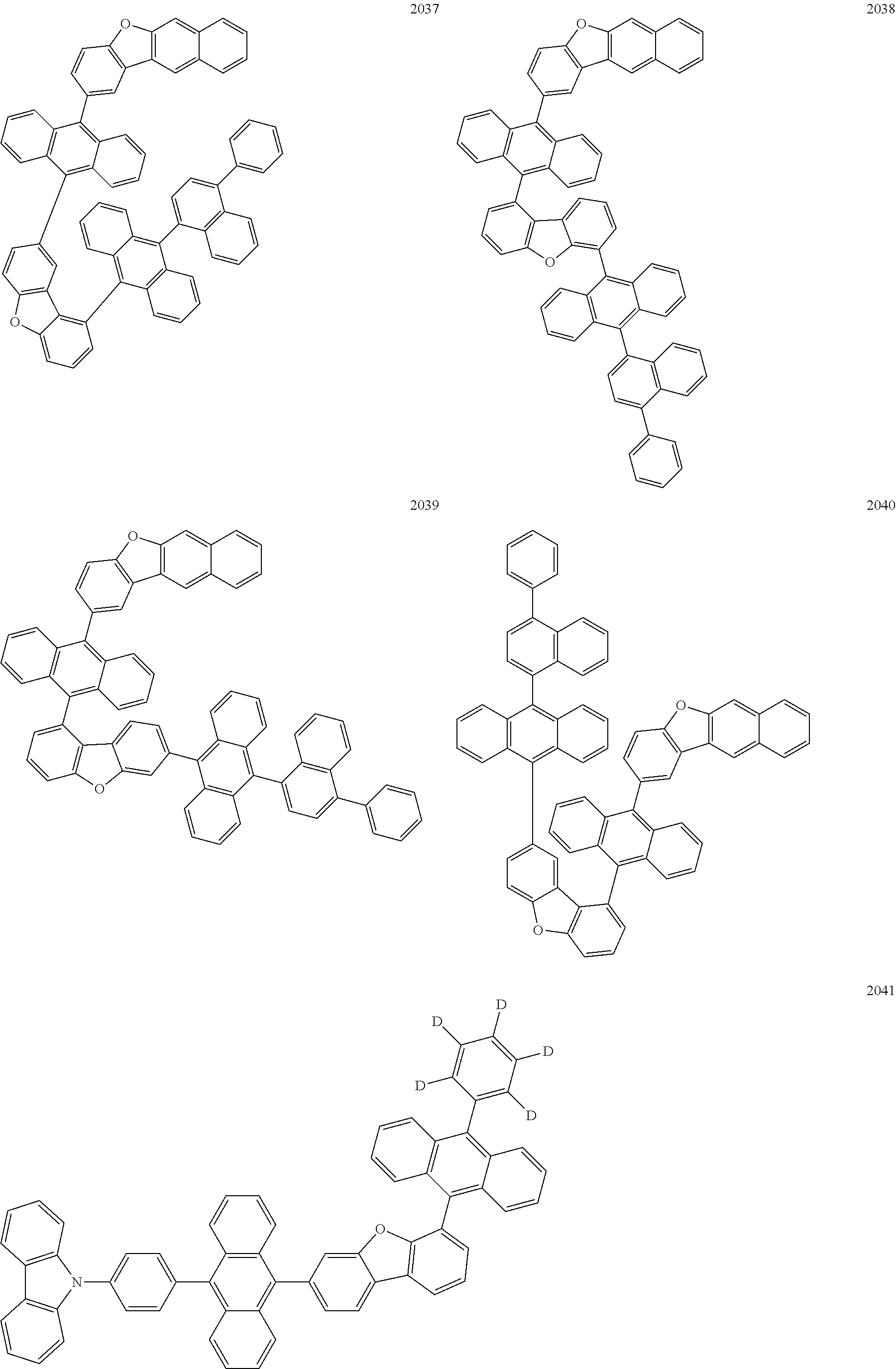

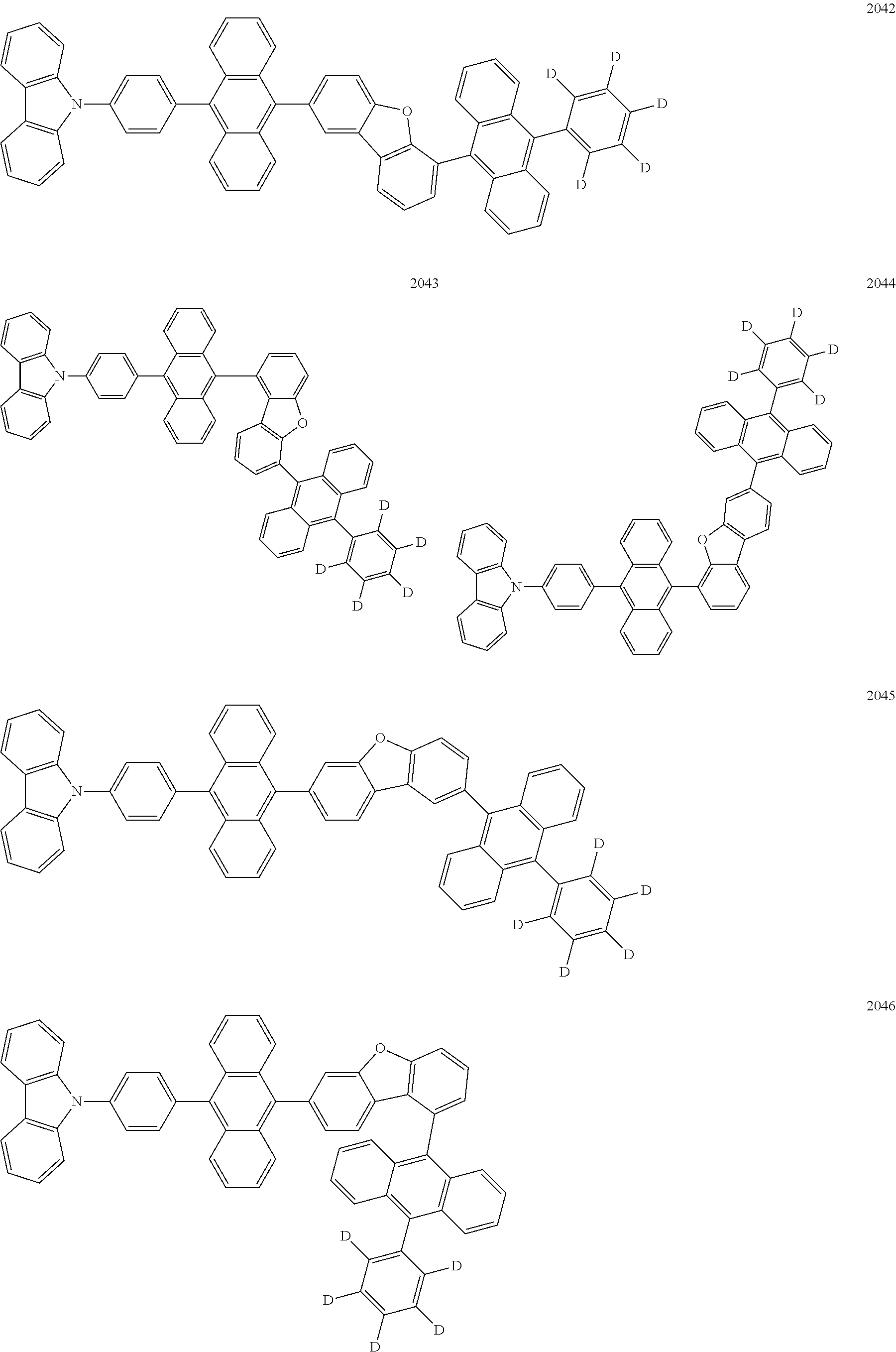

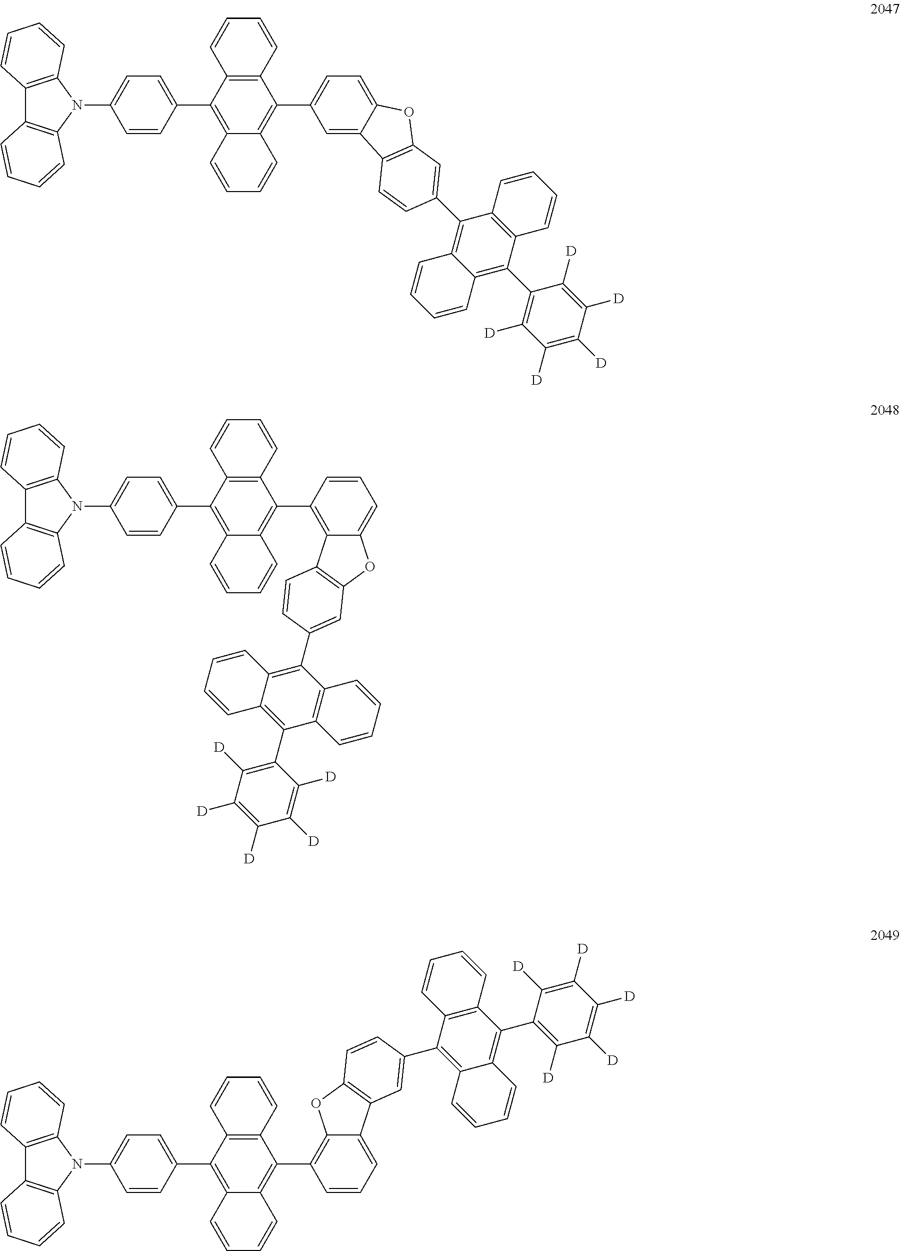 [SMILES: [2H]c1c([2H])c([2H])c(-c2c3ccccc3c(-c3ccc4c(c3)oc3ccc(-c5c6ccccc6c(-c6ccc(-n7c8ccccc8c8ccccc87)cc6)c6ccccc56)cc34)c3ccccc23)c([2H])c1[2H] ; [2H]c1c([2H])c([2H])c(-c2c3ccccc3c(-c3ccc4c(c3)oc3cccc(-c5c6ccccc6c(-c6ccc(-n7c8ccccc8c8ccccc87)cc6)c6ccccc56)c34)c3ccccc23)c([2H])c1[2H] ; [2H]c1c([2H])c([2H])c(-c2c3ccccc3c(-c3ccc4oc5c(-c6c7ccccc7c(-c7ccc(-n8c9ccccc9c9ccccc98)cc7)c7ccccc67)cccc5c4c3)c3ccccc23)c([2H])c1[2H]]

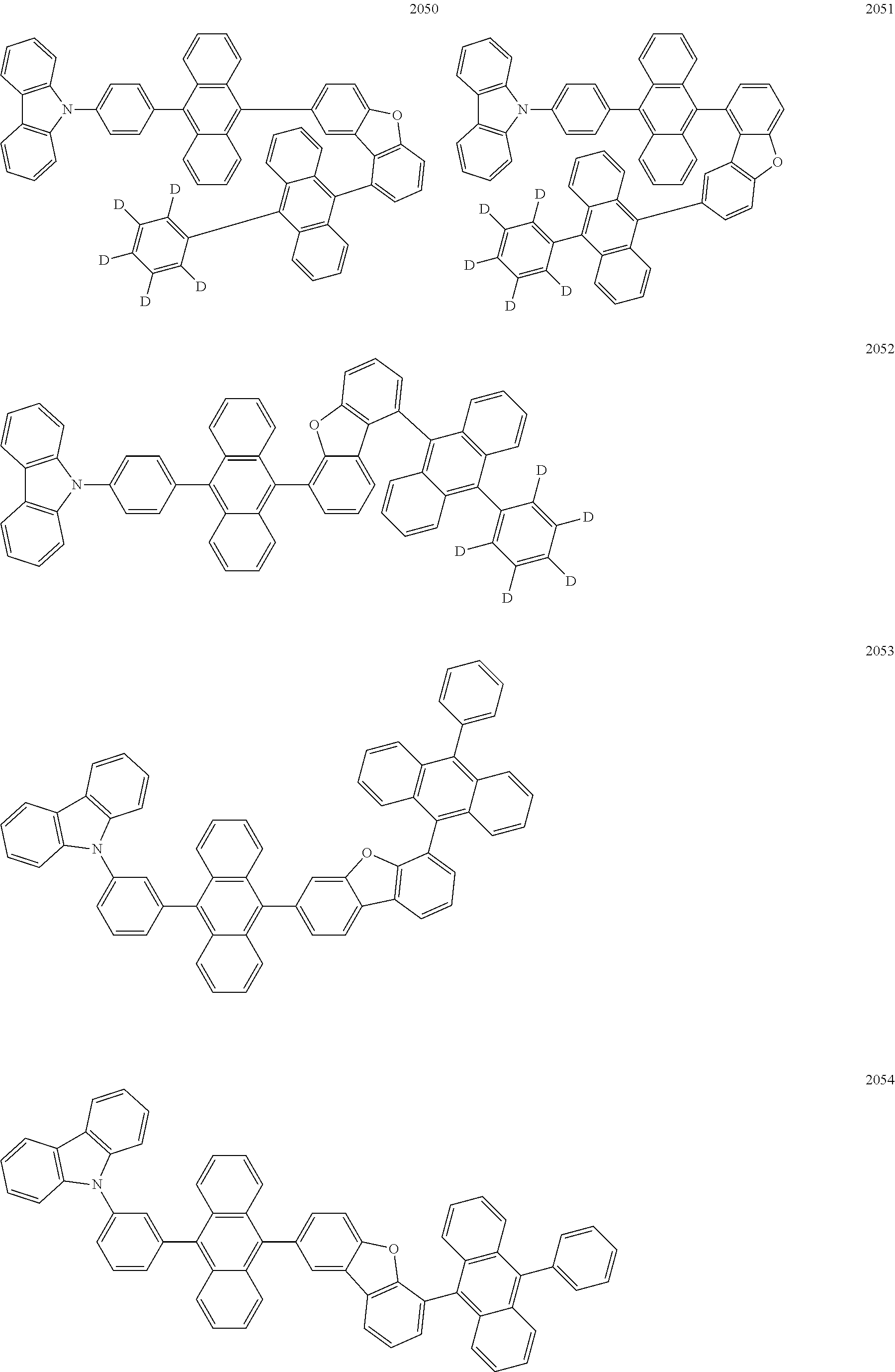

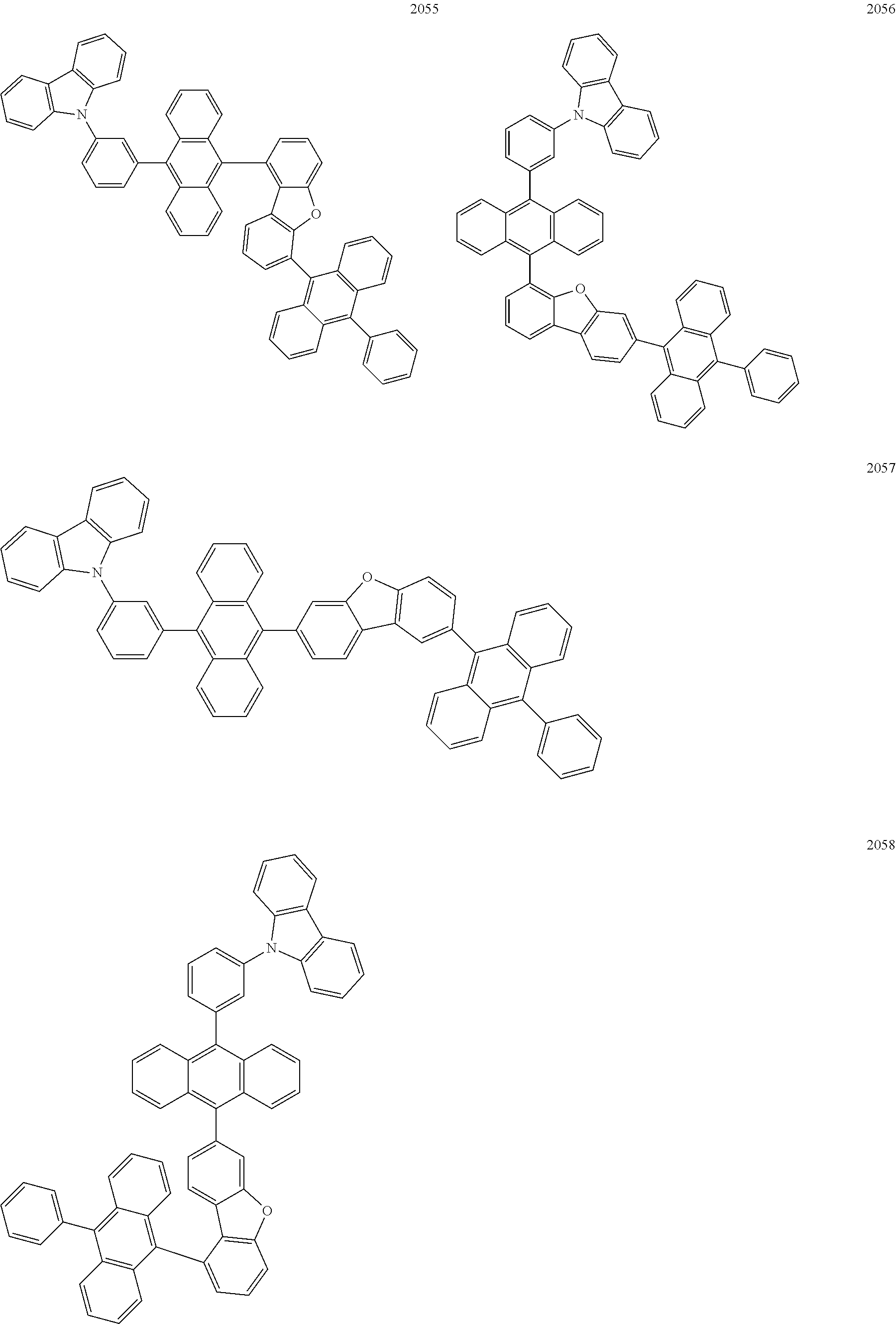 [SMILES: c1ccc(-c2c3ccccc3c(-c3cccc4c3oc3cccc(-c5c6ccccc6c(-c6cccc(-n7c8ccccc8c8ccccc87)c6)c6ccccc56)c34)c3ccccc23)cc1 ; c1ccc(-c2c3ccccc3c(-c3ccc4c(c3)oc3c(-c5c6ccccc6c(-c6cccc(-n7c8ccccc8c8ccccc87)c6)c6ccccc56)cccc34)c3ccccc23)cc1 ; c1ccc(-c2c3ccccc3c(-c3ccc4oc5cc(-c6c7ccccc7c(-c7cccc(-n8c9ccccc9c9ccccc98)c7)c7ccccc67)ccc5c4c3)c3ccccc23)cc1 ; c1ccc(-c2c3ccccc3c(-c3cccc4oc5cc(-c6c7ccccc7c(-c7cccc(-n8c9ccccc9c9ccccc98)c7)c7ccccc67)ccc5c34)c3ccccc23)cc1]

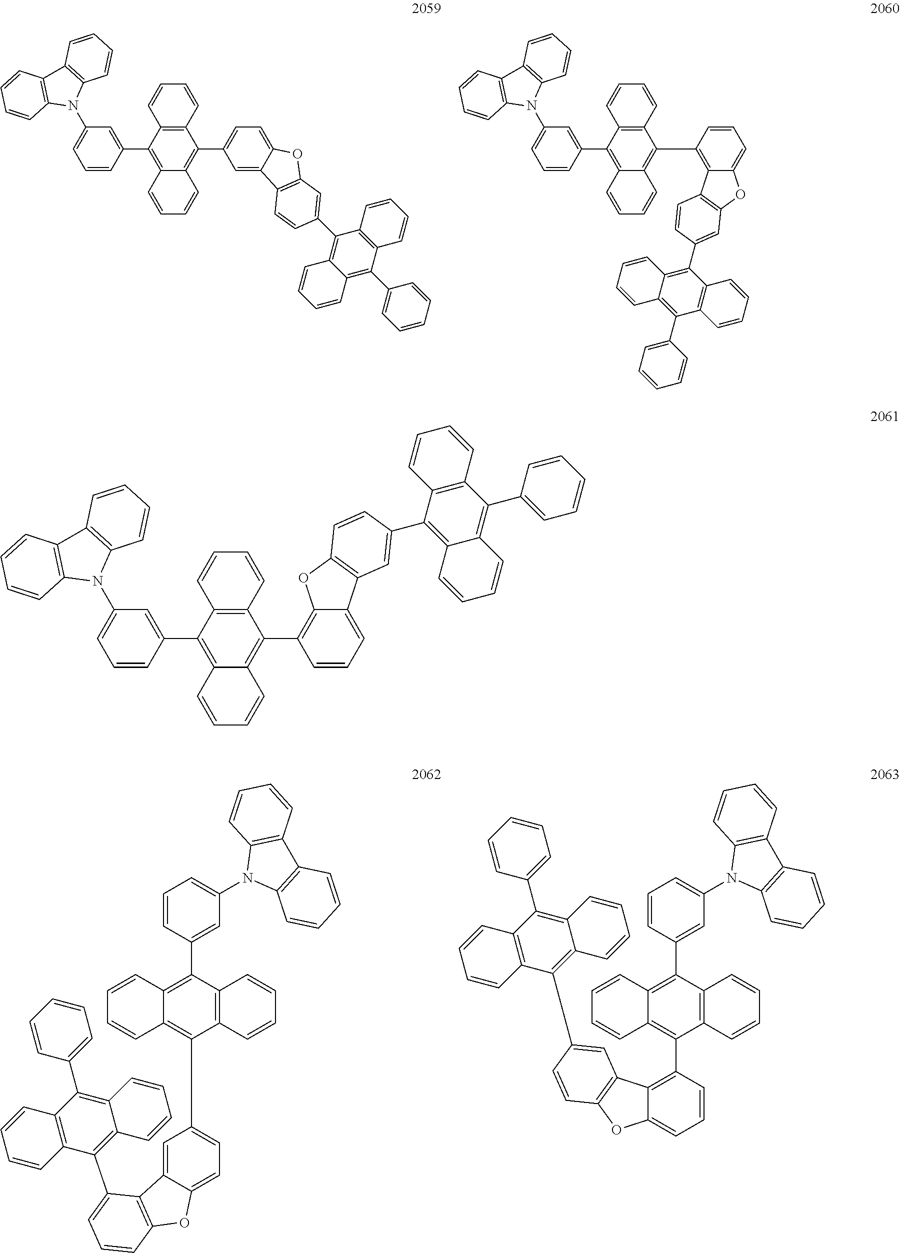

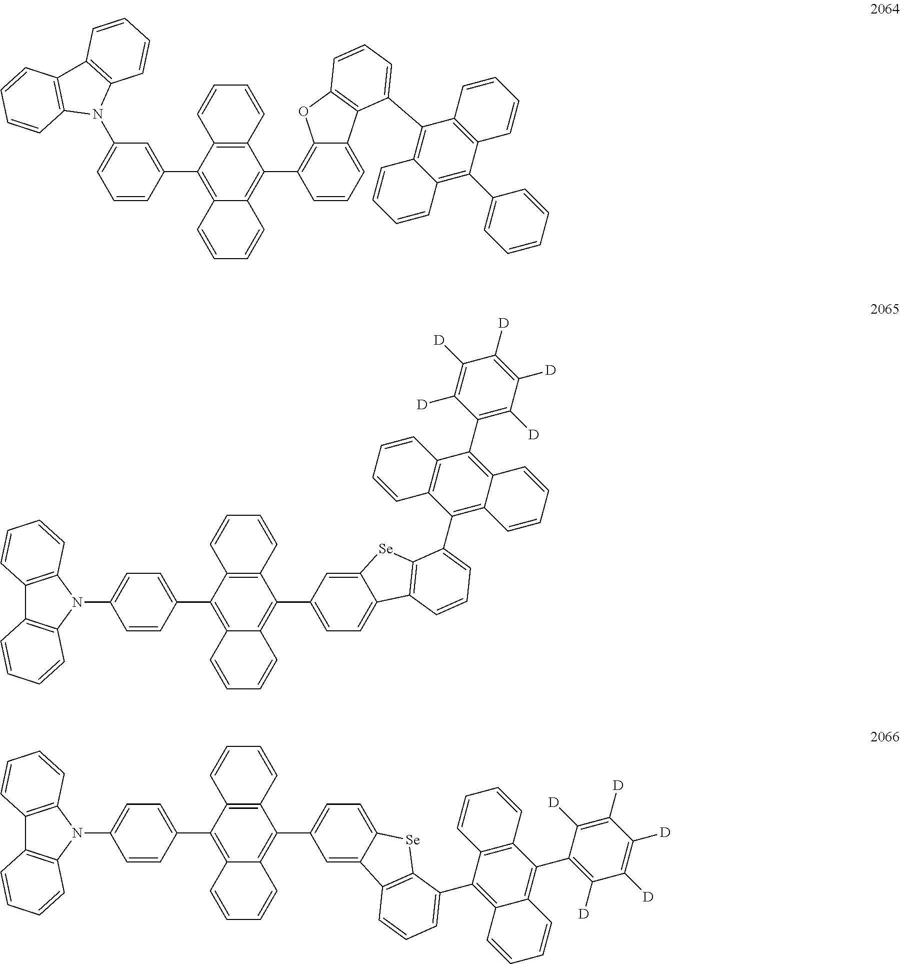 [SMILES: c1ccc(-c2c3ccccc3c(-c3cccc4oc5c(-c6c7ccccc7c(-c7cccc(-n8c9ccccc9c9ccccc98)c7)c7ccccc67)cccc5c34)c3ccccc23)cc1 ; [2H]c1c([2H])c([2H])c(-c2c3ccccc3c(-c3cccc4c3[se]c3cc(-c5c6ccccc6c(-c6ccc(-n7c8ccccc8c8ccccc87)cc6)c6ccccc56)ccc34)c3ccccc23)c([2H])c1[2H] ; [2H]c1c([2H])c([2H])c(-c2c3ccccc3c(-c3cccc4c3[se]c3ccc(-c5c6ccccc6c(-c6ccc(-n7c8ccccc8c8ccccc87)cc6)c6ccccc56)cc34)c3ccccc23)c([2H])c1[2H]]

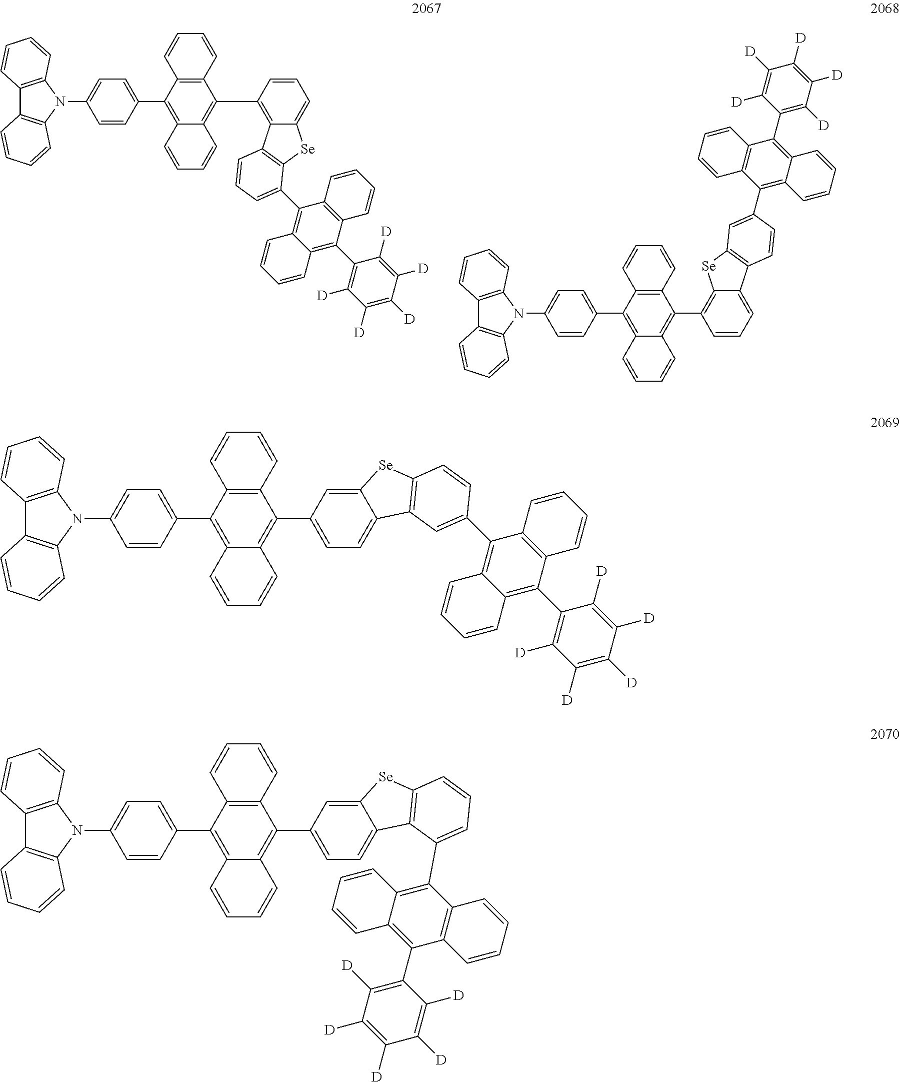

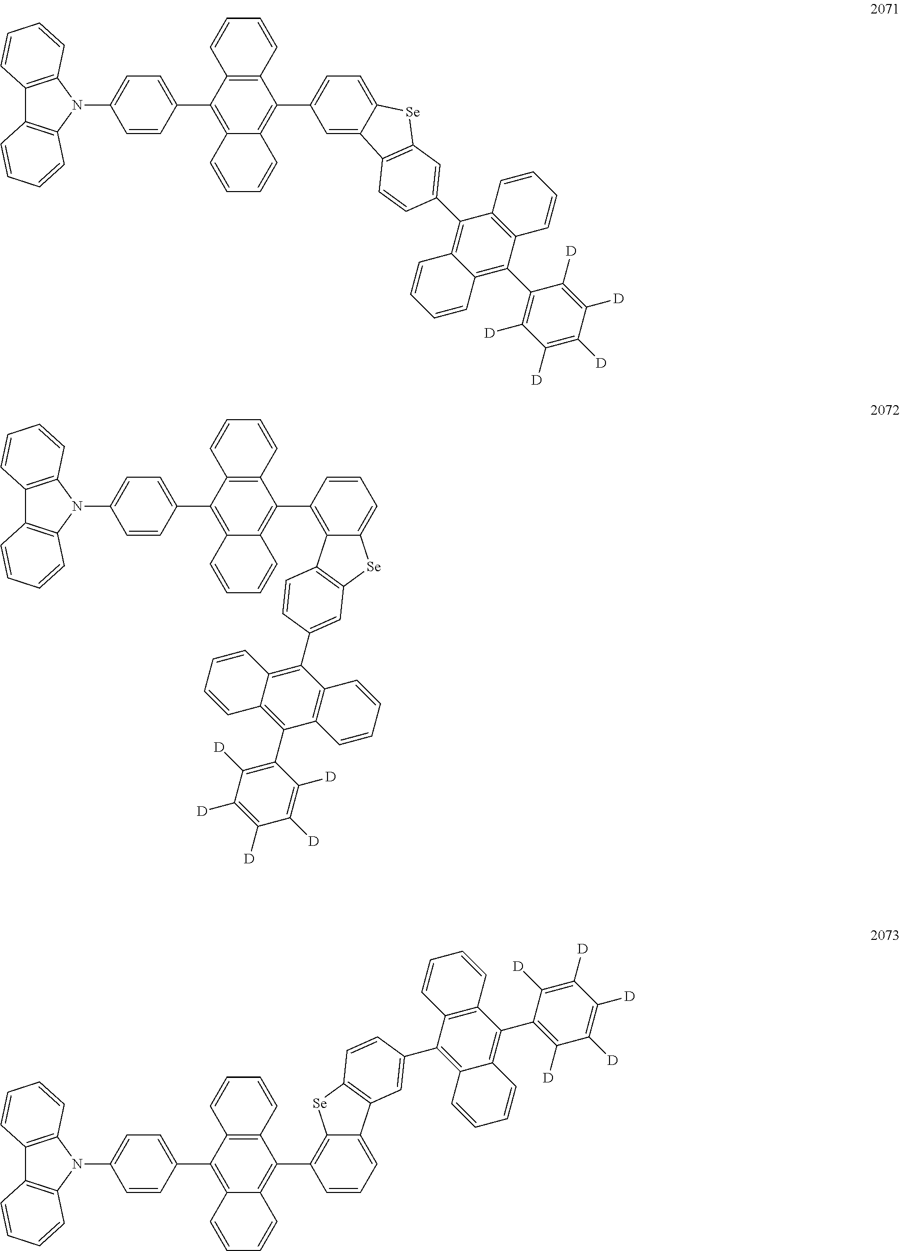 [SMILES: [2H]c1c([2H])c([2H])c(-c2c3ccccc3c(-c3ccc4c(c3)[se]c3ccc(-c5c6ccccc6c(-c6ccc(-n7c8ccccc8c8ccccc87)cc6)c6ccccc56)cc34)c3ccccc23)c([2H])c1[2H] ; [2H]c1c([2H])c([2H])c(-c2c3ccccc3c(-c3ccc4c(c3)[se]c3cccc(-c5c6ccccc6c(-c6ccc(-n7c8ccccc8c8ccccc87)cc6)c6ccccc56)c34)c3ccccc23)c([2H])c1[2H] ; [2H]c1c([2H])c([2H])c(-c2c3ccccc3c(-c3ccc4[se]c5c(-c6c7ccccc7c(-c7ccc(-n8c9ccccc9c9ccccc98)cc7)c7ccccc67)cccc5c4c3)c3ccccc23)c([2H])c1[2H]]

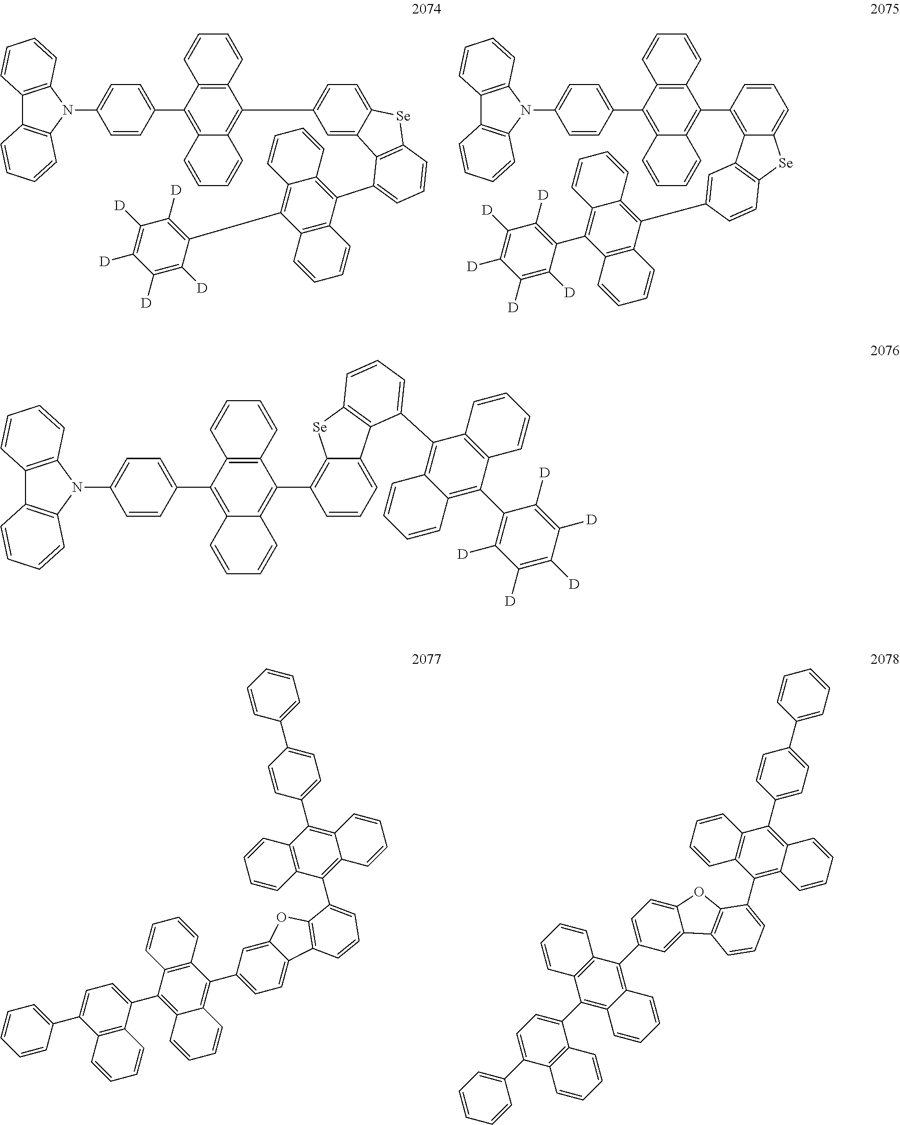

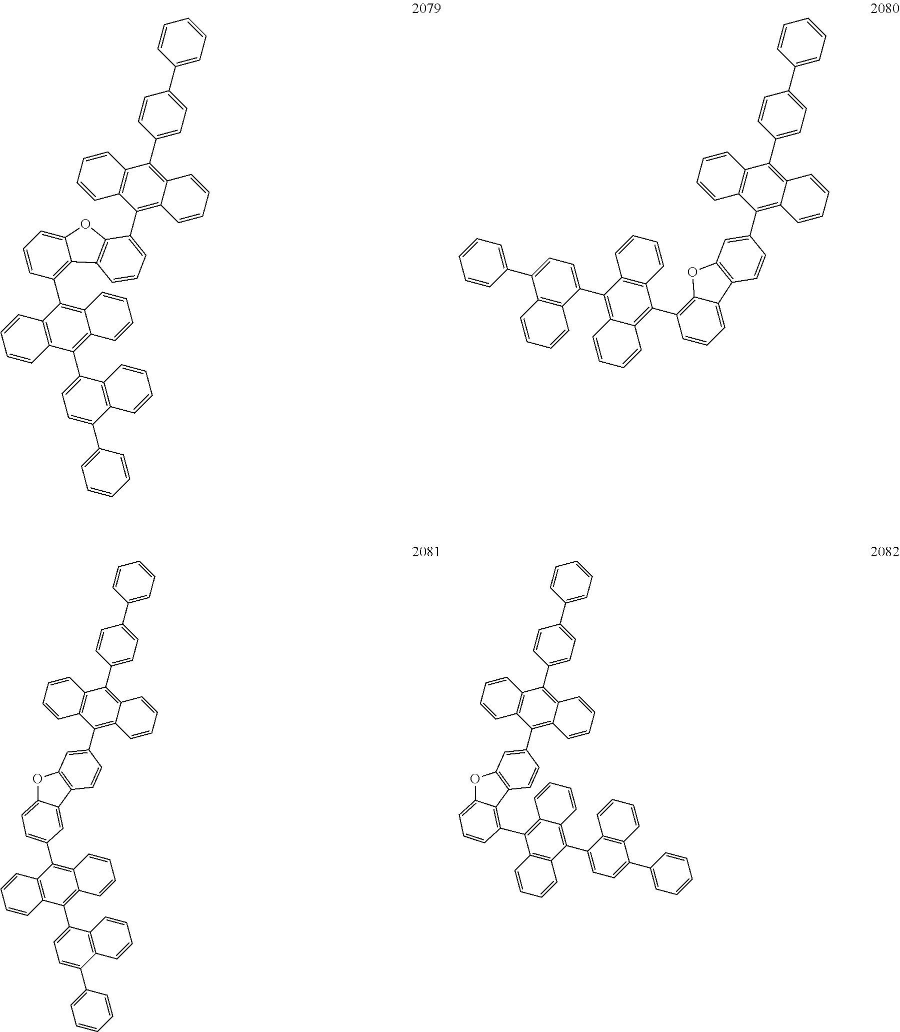 [SMILES: c1ccc(-c2ccc(-c3c4ccccc4c(-c4cccc5c4oc4cccc(-c6c7ccccc7c(-c7ccc(-c8ccccc8)c8ccccc78)c7ccccc67)c45)c4ccccc34)cc2)cc1 ; c1ccc(-c2ccc(-c3c4ccccc4c(-c4ccc5c(c4)oc4c(-c6c7ccccc7c(-c7ccc(-c8ccccc8)c8ccccc78)c7ccccc67)cccc45)c4ccccc34)cc2)cc1 ; c1ccc(-c2ccc(-c3c4ccccc4c(-c4ccc5c(c4)oc4ccc(-c6c7ccccc7c(-c7ccc(-c8ccccc8)c8ccccc78)c7ccccc67)cc45)c4ccccc34)cc2)cc1 ; c1ccc(-c2ccc(-c3c4ccccc4c(-c4ccc5c(c4)oc4cccc(-c6c7ccccc7c(-c7ccc(-c8ccccc8)c8ccccc78)c7ccccc67)c45)c4ccccc34)cc2)cc1]

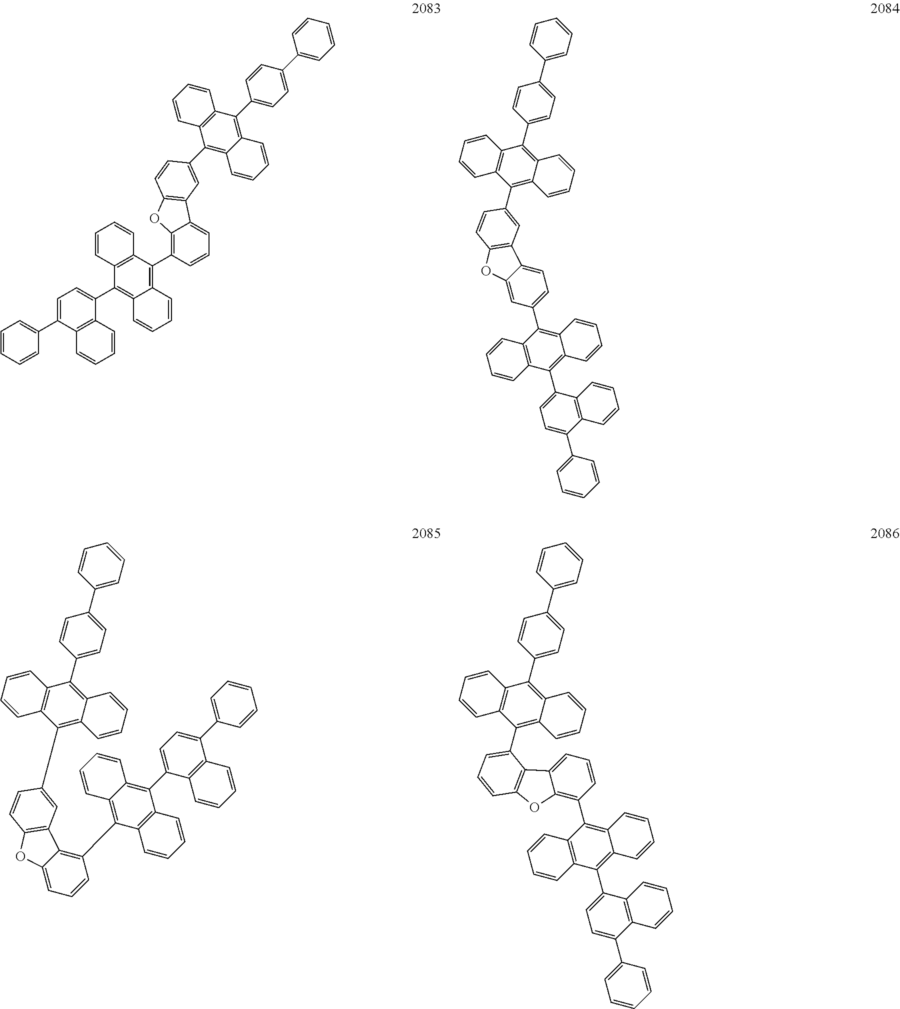 [SMILES: c1ccc(-c2ccc(-c3c4ccccc4c(-c4ccc5oc6c(-c7c8ccccc8c(-c8ccc(-c9ccccc9)c9ccccc89)c8ccccc78)cccc6c5c4)c4ccccc34)cc2)cc1 ; c1ccc(-c2ccc(-c3c4ccccc4c(-c4ccc5oc6cc(-c7c8ccccc8c(-c8ccc(-c9ccccc9)c9ccccc89)c8ccccc78)ccc6c5c4)c4ccccc34)cc2)cc1 ; c1ccc(-c2ccc(-c3c4ccccc4c(-c4ccc5oc6cccc(-c7c8ccccc8c(-c8ccc(-c9ccccc9)c9ccccc89)c8ccccc78)c6c5c4)c4ccccc34)cc2)cc1 ; c1ccc(-c2ccc(-c3c4ccccc4c(-c4cccc5oc6c(-c7c8ccccc8c(-c8ccc(-c9ccccc9)c9ccccc89)c8ccccc78)cccc6c45)c4ccccc34)cc2)cc1]

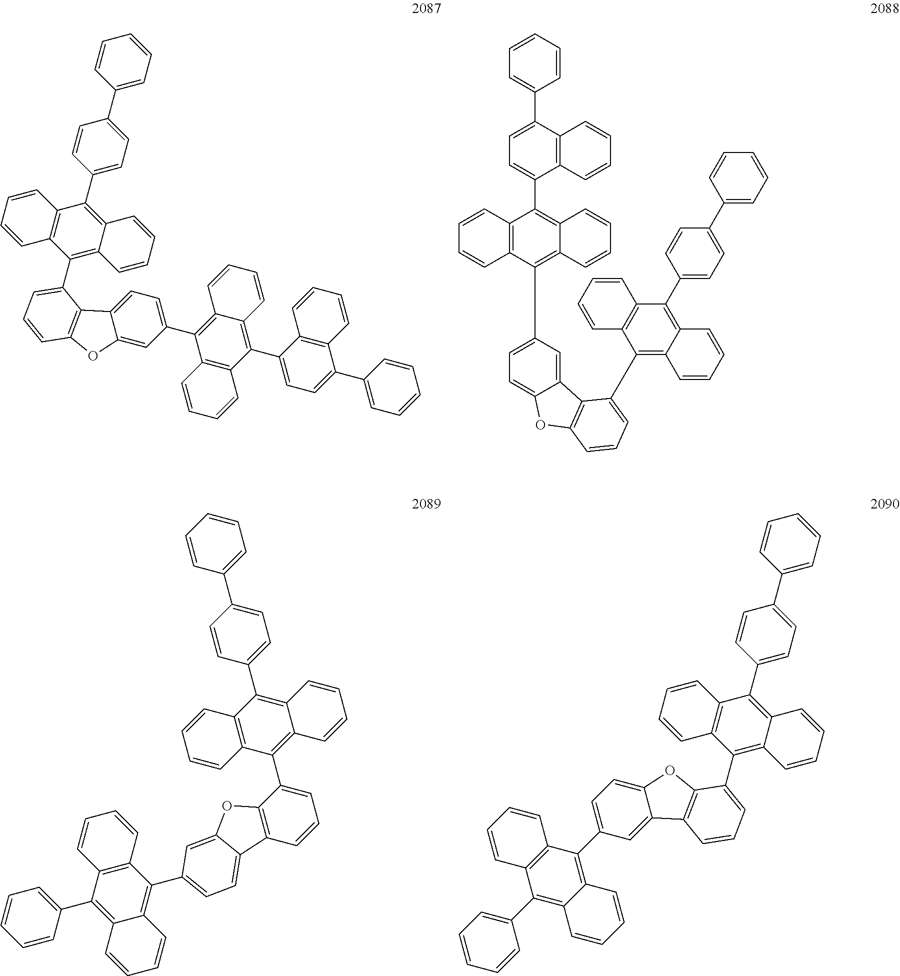 [SMILES: c1ccc(-c2ccc(-c3c4ccccc4c(-c4cccc5oc6cc(-c7c8ccccc8c(-c8ccc(-c9ccccc9)c9ccccc89)c8ccccc78)ccc6c45)c4ccccc34)cc2)cc1 ; c1ccc(-c2ccc(-c3c4ccccc4c(-c4cccc5oc6ccc(-c7c8ccccc8c(-c8ccc(-c9ccccc9)c9ccccc89)c8ccccc78)cc6c45)c4ccccc34)cc2)cc1 ; c1ccc(-c2ccc(-c3c4ccccc4c(-c4cccc5c4oc4cc(-c6c7ccccc7c(-c7ccccc7)c7ccccc67)ccc45)c4ccccc34)cc2)cc1 ; c1ccc(-c2ccc(-c3c4ccccc4c(-c4cccc5c4oc4ccc(-c6c7ccccc7c(-c7ccccc7)c7ccccc67)cc45)c4ccccc34)cc2)cc1]

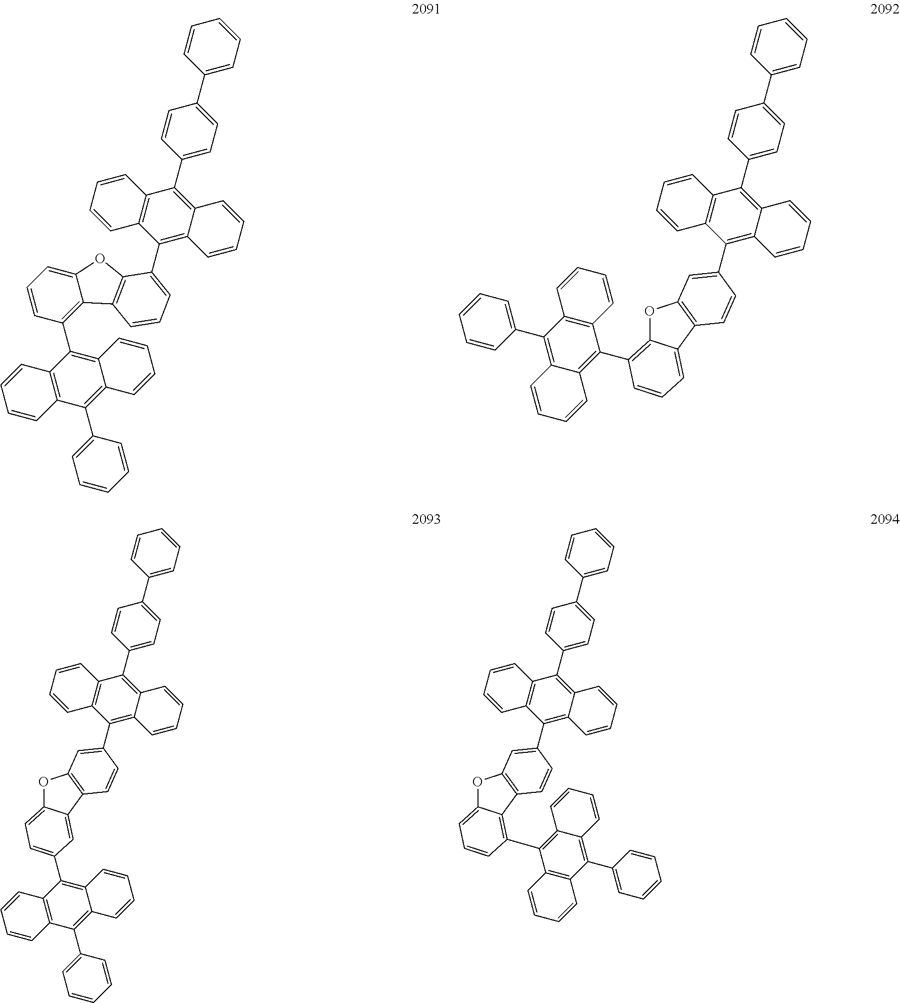 [SMILES: c1ccc(-c2ccc(-c3c4ccccc4c(-c4cccc5c4oc4cccc(-c6c7ccccc7c(-c7ccccc7)c7ccccc67)c45)c4ccccc34)cc2)cc1 ; c1ccc(-c2ccc(-c3c4ccccc4c(-c4ccc5c(c4)oc4c(-c6c7ccccc7c(-c7ccccc7)c7ccccc67)cccc45)c4ccccc34)cc2)cc1 ; c1ccc(-c2ccc(-c3c4ccccc4c(-c4ccc5c(c4)oc4ccc(-c6c7ccccc7c(-c7ccccc7)c7ccccc67)cc45)c4ccccc34)cc2)cc1 ; c1ccc(-c2ccc(-c3c4ccccc4c(-c4ccc5c(c4)oc4cccc(-c6c7ccccc7c(-c7ccccc7)c7ccccc67)c45)c4ccccc34)cc2)cc1]

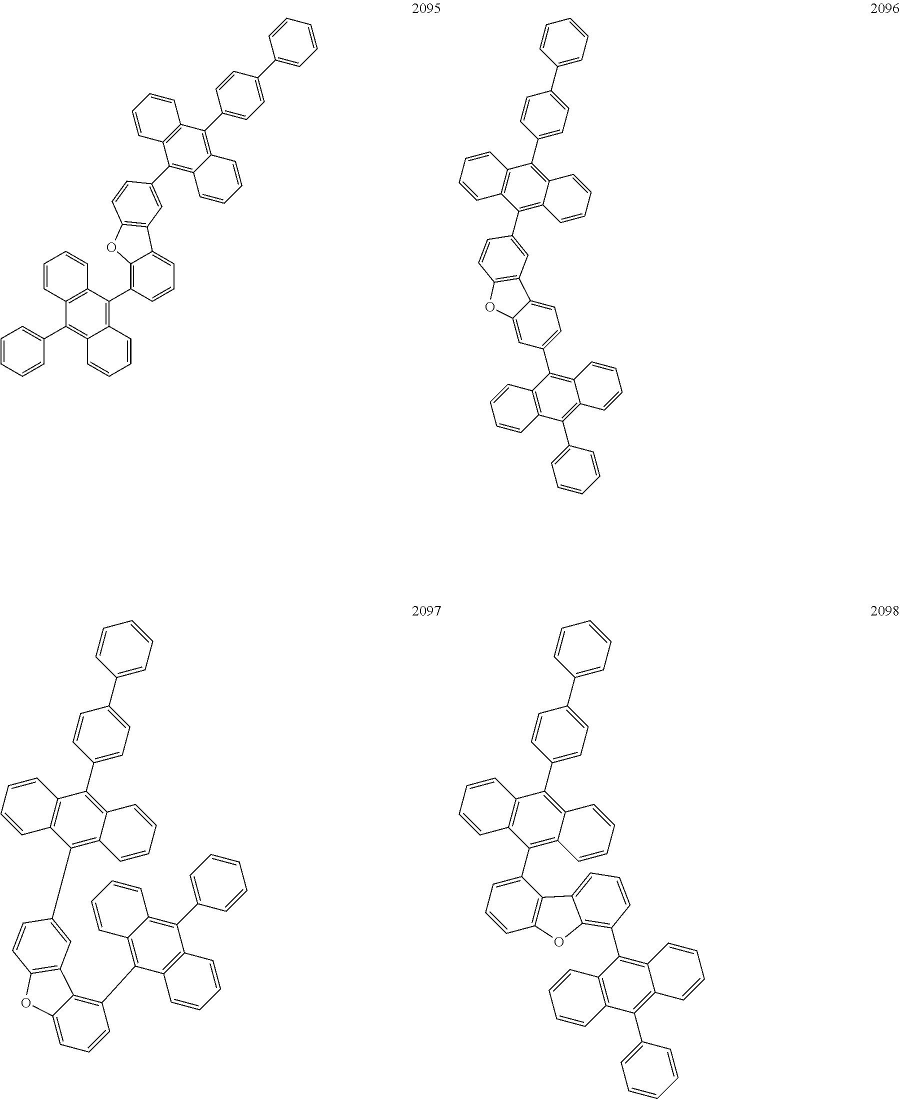 [SMILES: c1ccc(-c2ccc(-c3c4ccccc4c(-c4ccc5oc6c(-c7c8ccccc8c(-c8ccccc8)c8ccccc78)cccc6c5c4)c4ccccc34)cc2)cc1 ; c1ccc(-c2ccc(-c3c4ccccc4c(-c4ccc5oc6cc(-c7c8ccccc8c(-c8ccccc8)c8ccccc78)ccc6c5c4)c4ccccc34)cc2)cc1 ; c1ccc(-c2ccc(-c3c4ccccc4c(-c4ccc5oc6cccc(-c7c8ccccc8c(-c8ccccc8)c8ccccc78)c6c5c4)c4ccccc34)cc2)cc1 ; c1ccc(-c2ccc(-c3c4ccccc4c(-c4cccc5oc6c(-c7c8ccccc8c(-c8ccccc8)c8ccccc78)cccc6c45)c4ccccc34)cc2)cc1]

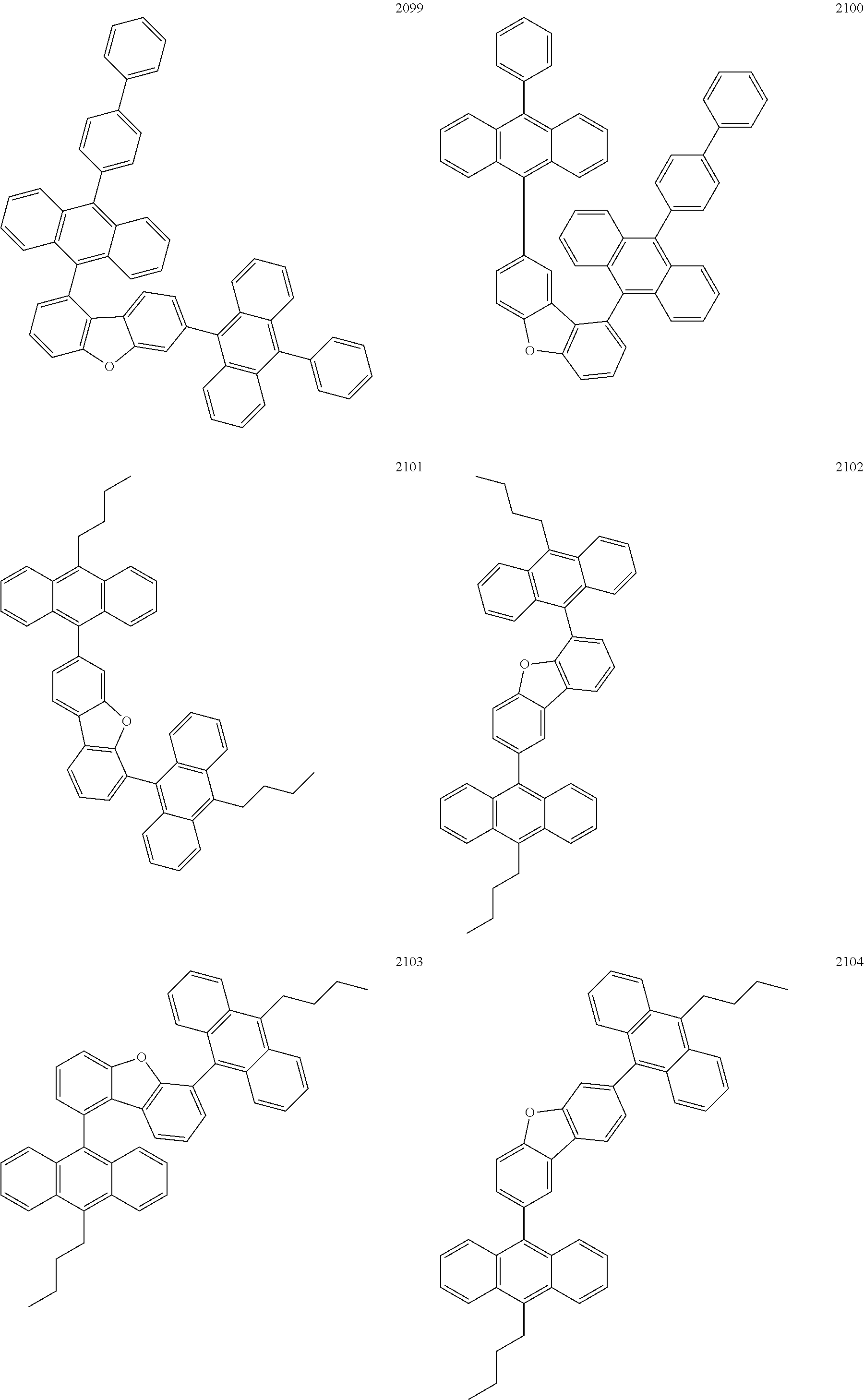

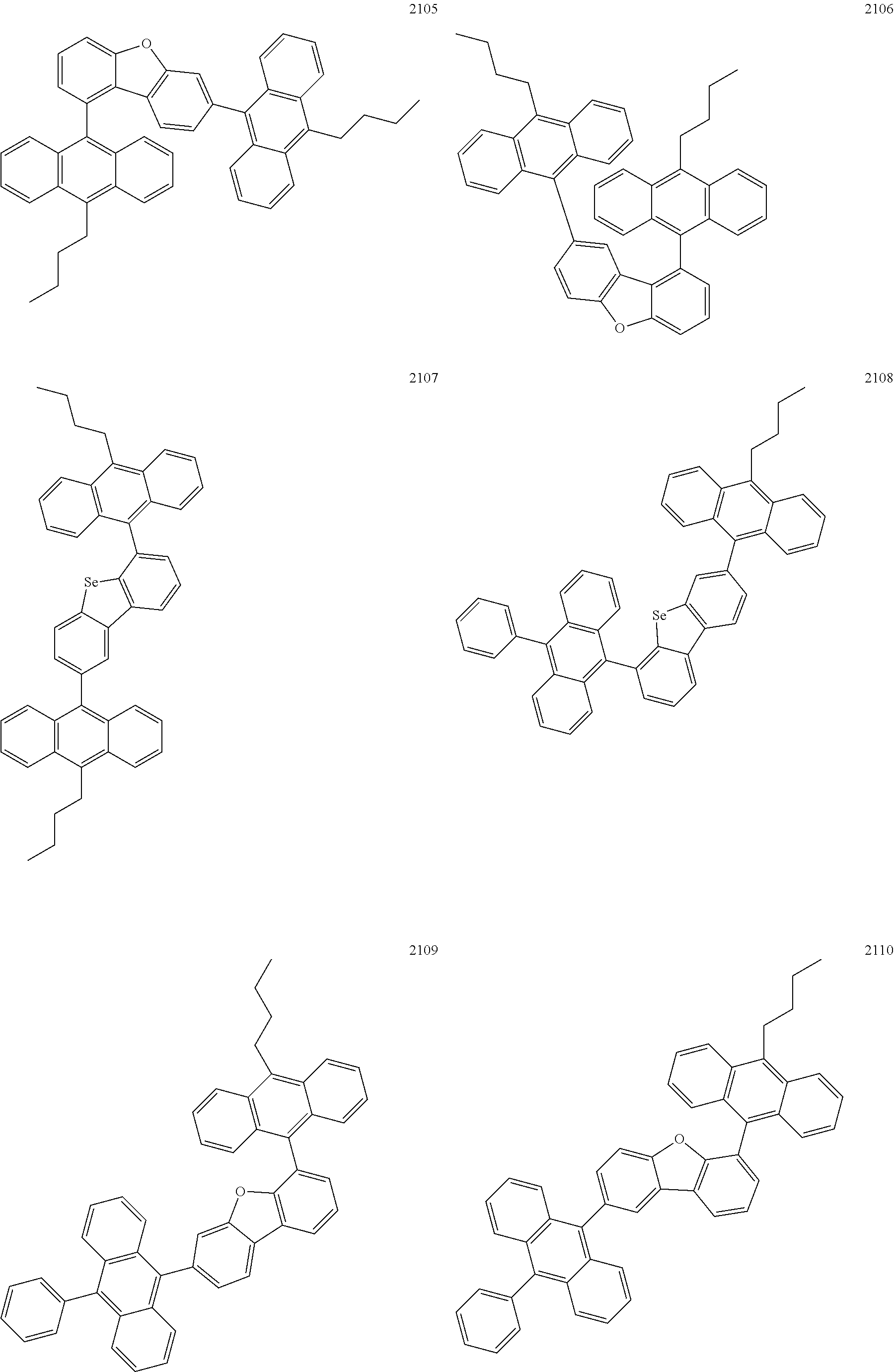 [SMILES: CCCCc1c2ccccc2c(-c2ccc3c(c2)oc2cccc(-c4c5ccccc5c(CCCC)c5ccccc45)c23)c2ccccc12 ; CCCCc1c2ccccc2c(-c2ccc3oc4cccc(-c5c6ccccc6c(CCCC)c6ccccc56)c4c3c2)c2ccccc12 ; CCCCc1c2ccccc2c(-c2ccc3[se]c4c(-c5c6ccccc6c(CCCC)c6ccccc56)cccc4c3c2)c2ccccc12 ; CCCCc1c2ccccc2c(-c2ccc3c(c2)[se]c2c(-c4c5ccccc5c(-c5ccccc5)c5ccccc45)cccc23)c2ccccc12 ; CCCCc1c2ccccc2c(-c2cccc3c2oc2cc(-c4c5ccccc5c(-c5ccccc5)c5ccccc45)ccc23)c2ccccc12 ; CCCCc1c2ccccc2c(-c2cccc3c2oc2ccc(-c4c5ccccc5c(-c5ccccc5)c5ccccc45)cc23)c2ccccc12]

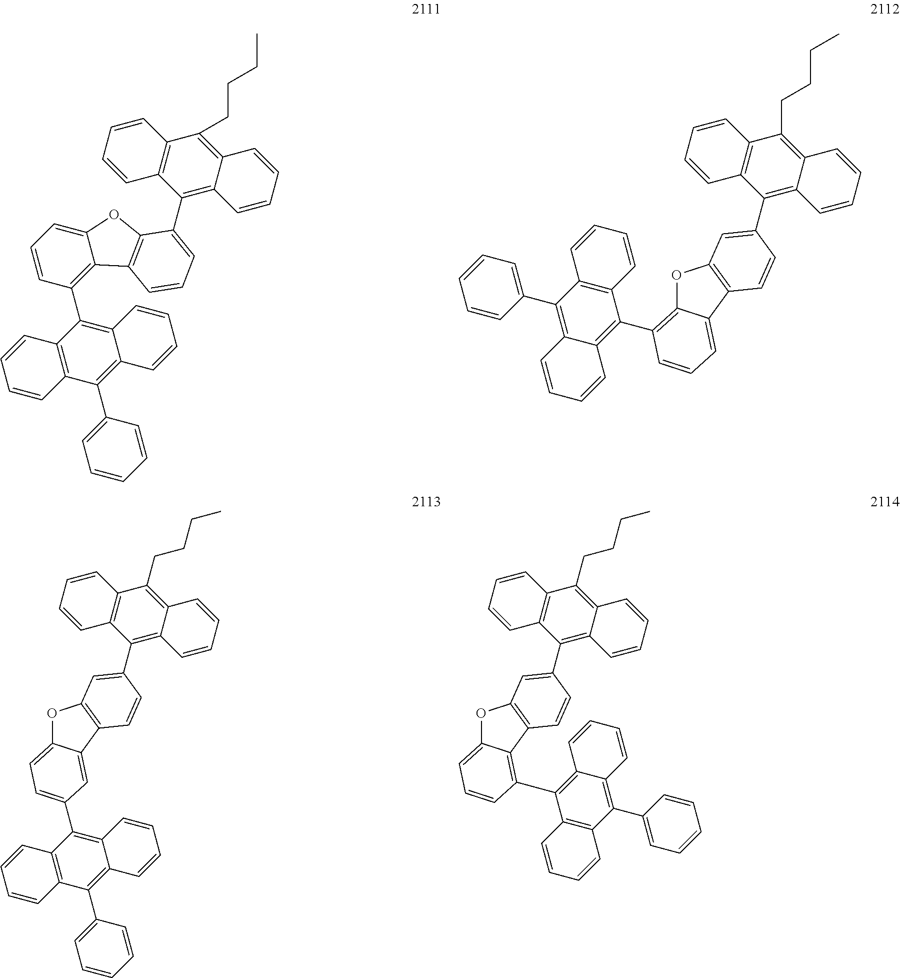 [SMILES: CCCCc1c2ccccc2c(-c2cccc3c2oc2cccc(-c4c5ccccc5c(-c5ccccc5)c5ccccc45)c23)c2ccccc12 ; CCCCc1c2ccccc2c(-c2ccc3c(c2)oc2c(-c4c5ccccc5c(-c5ccccc5)c5ccccc45)cccc23)c2ccccc12 ; CCCCc1c2ccccc2c(-c2ccc3c(c2)oc2ccc(-c4c5ccccc5c(-c5ccccc5)c5ccccc45)cc23)c2ccccc12 ; CCCCc1c2ccccc2c(-c2ccc3c(c2)oc2cccc(-c4c5ccccc5c(-c5ccccc5)c5ccccc45)c23)c2ccccc12]

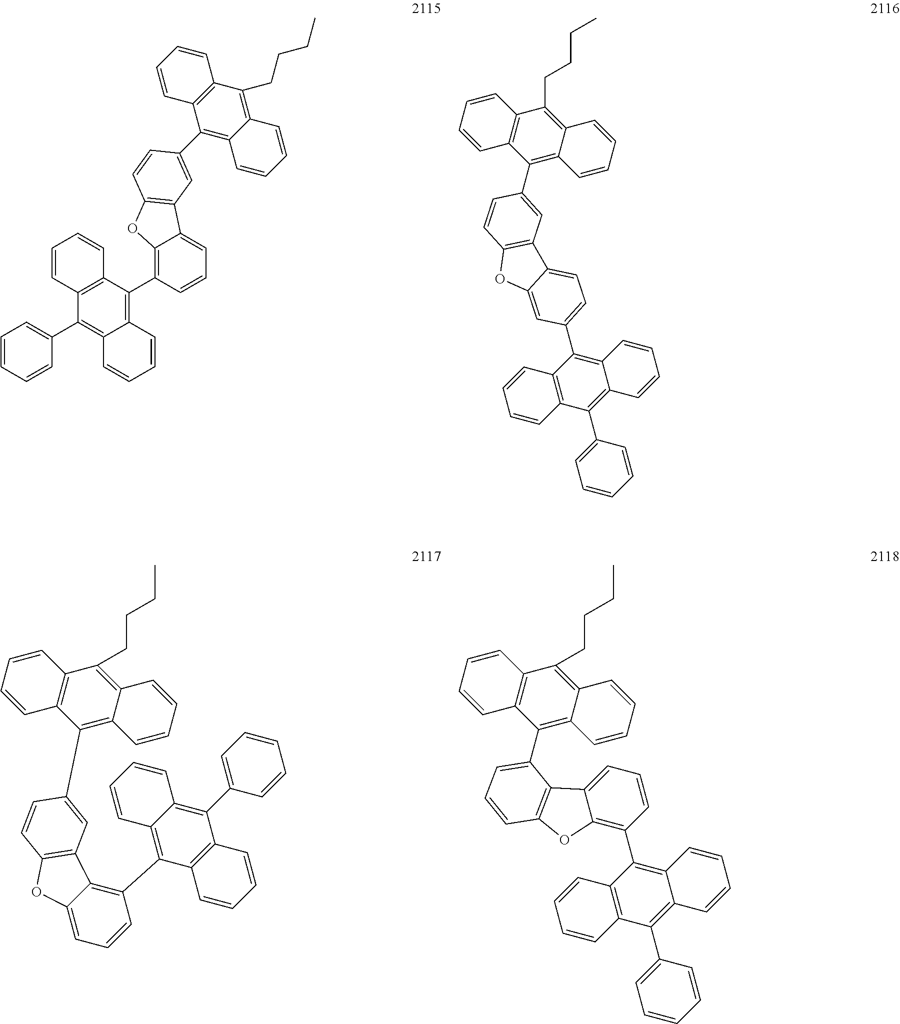 [SMILES: CCCCc1c2ccccc2c(-c2ccc3oc4c(-c5c6ccccc6c(-c6ccccc6)c6ccccc56)cccc4c3c2)c2ccccc12 ; CCCCc1c2ccccc2c(-c2ccc3oc4cc(-c5c6ccccc6c(-c6ccccc6)c6ccccc56)ccc4c3c2)c2ccccc12 ; CCCCc1c2ccccc2c(-c2ccc3oc4cccc(-c5c6ccccc6c(-c6ccccc6)c6ccccc56)c4c3c2)c2ccccc12 ; CCCCc1c2ccccc2c(-c2cccc3oc4c(-c5c6ccccc6c(-c6ccccc6)c6ccccc56)cccc4c23)c2ccccc12]

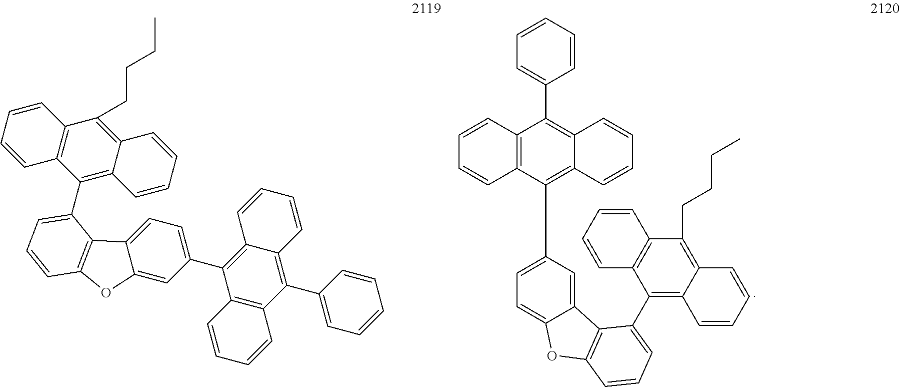

The heterocyclic compound represented by Formula 1 may satisfy the structure of Formula 1 and have an asymmetric structure, and thus, the crystallinity thereof may be decreased. Accordingly, in the heterocyclic compound, close packing between molecules may be prevented, and thus, the heterocyclic compound represented by Formula 1 may have the effect of obtaining relatively excellent amorphous thin-film characteristics.

As described above, the heterocyclic compound represented by Formula 1 may have such electrical characteristics suitable for use as a material for an organic light-emitting device, for example, a host material in the emission layer, a hole transport material, an electron transport material, and the like. Accordingly, an organic light-emitting device using the heterocyclic compound may have high efficiency and/or long lifespan.

In one or more embodiments, the heterocyclic compound represented by Formula 1 satisfies Equation 1:

E(T1)<E(S1)<2×E(T1). Equation 1

In Equation 1, E(T1) indicates the lowest excitation triplet energy level of the heterocyclic compound and E(S1) indicates the lowest excitation singlet energy level of the heterocyclic compound.

In one or more embodiments, the heterocyclic compound represented by Formula 1 satisfies Equation 2:

[2×E(T1)]−E(S1)<0.5 eV. Equation 2

In Equation 2, E(T1) indicates the lowest excitation triplet energy level of the heterocyclic compound and E(S1) indicates the lowest excitation singlet energy level of the heterocyclic compound.

Since the heterocyclic compound represented by Formula 1 satisfy Equation 1 and/or Equation 2, the triplet-triplet fusion (TTF) phenomenon in which triplet excitons are fused to generate singlet excitons may highly likely occur. Accordingly, when the heterocyclic compound is applied to an organic light-emitting device, fluorescent emission may occur from singlet excitons generated by the TTF phenomenon, thereby improving emission efficiency and lifetime of a device.

For example, the highest occupied molecular orbital (HOMO), lowest unoccupied molecular orbital (LUMO) and triplet energy level (E(T1)), singlet (E(S1)) energy levels of Compounds 1, 113, 134, 171, 1121, and 2102 are measured using the DFT method of the Gaussian program (B3LYP, structurally optimized at the level of 6-31G (d, p)). The evaluation results are shown in Table 1 below.

TABLE 1

| Compound | HOMO | LUMO | E(S1) | E(T1) | 2E(T1) − E(S1) |
|---|---|---|---|---|---|
| No. | (eV) | (eV) | (eV) | (eV) | (eV) |
| Compound 1 | −5.12 | −1.64 | 3.13 | 1.73 | 0.33 |
| Compound 113 | −5.13 | −1.64 | 3.15 | 1.74 | 0.33 |
| Compound 134 | −5.29 | −1.82 | 3.14 | 1.73 | 0.32 |
| Compound 171 | −5.13 | −1.67 | 3.13 | 1.73 | 0.33 |
| Compound 1121 | −5.13 | −1.70 | 3.12 | 1.73 | 0.34 |
| Compound 2102 | −5.08 | −1.63 | 3.11 | 1.71 | 0.31 |

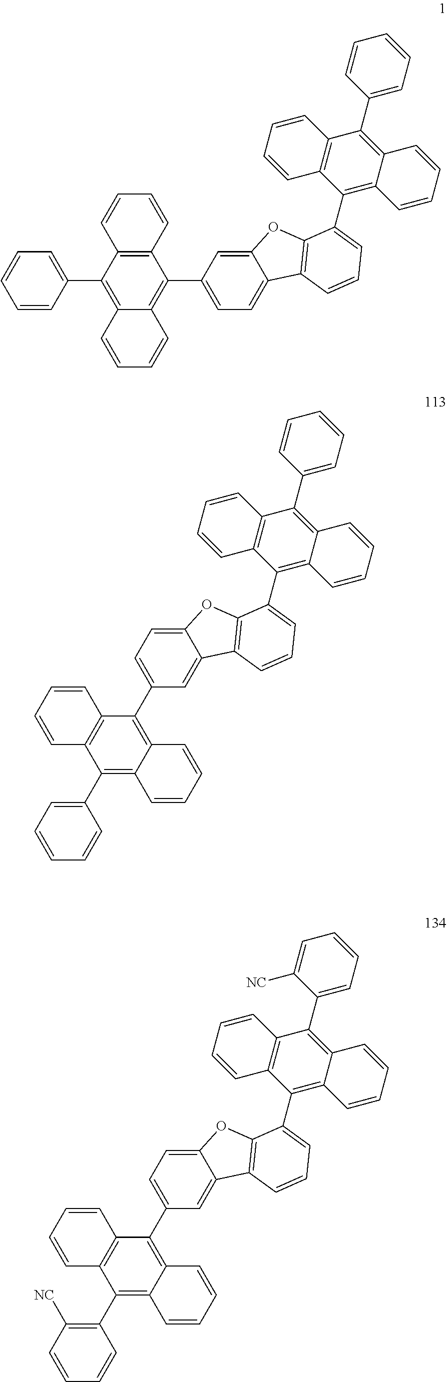 [SMILES: c1ccc(-c2c3ccccc3c(-c3ccc4c(c3)oc3c(-c5c6ccccc6c(-c6ccccc6)c6ccccc56)cccc34)c3ccccc23)cc1 ; c1ccc(-c2c3ccccc3c(-c3ccc4oc5c(-c6c7ccccc7c(-c7ccccc7)c7ccccc67)cccc5c4c3)c3ccccc23)cc1 ; N#Cc1ccccc1-c1c2ccccc2c(-c2ccc3oc4c(-c5c6ccccc6c(-c6ccccc6C#N)c6ccccc56)cccc4c3c2)c2ccccc12]

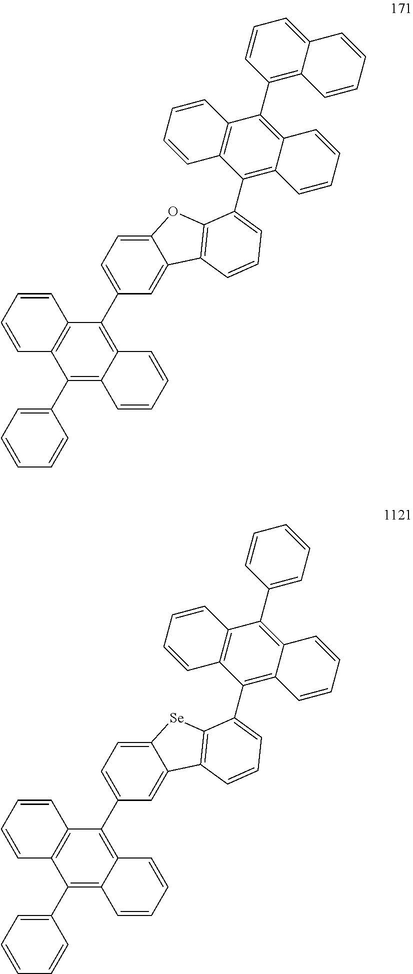 [SMILES: c1ccc(-c2c3ccccc3c(-c3ccc4oc5c(-c6c7ccccc7c(-c7cccc8ccccc78)c7ccccc67)cccc5c4c3)c3ccccc23)cc1 ; c1ccc(-c2c3ccccc3c(-c3ccc4[se]c5c(-c6c7ccccc7c(-c7ccccc7)c7ccccc67)cccc5c4c3)c3ccccc23)cc1]

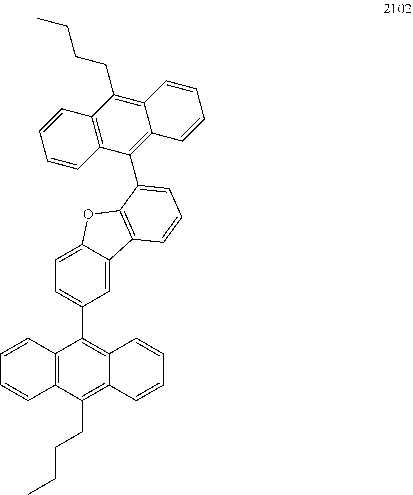 [SMILES: CCCCc1c2ccccc2c(-c2ccc3oc4c(-c5c6ccccc6c(CCCC)c6ccccc56)cccc4c3c2)c2ccccc12]

From the Table 1, it can be seen that the heterocyclic compound represented by Formula 1 has electric characteristics suitable for use in an electronic device, for example, as a material for an emission layer of an organic light-emitting device.

Synthesis method of the heterocyclic compound represented by Formula 1 may be recognized by those skilled in the art with reference to the following Synthesis Examples.

The heterocyclic compound represented by Formula 1 may be suitable for use in an organic layer, for example, as an emission layer material, a hole transport region material, and/or an electron transport region material of an organic layer. Accordingly, another aspect of the present disclosure provides an organic light-emitting device including: a first electrode; a second electrode; and an organic layer located between the first electrode and the second electrode and including an emission layer, wherein the organic layer may include at least one heterocyclic compound represented by Formula 1.

The organic light-emitting device may have a low driving voltage, high efficiency, high luminance, high quantum emission efficiency, and long lifespan, due to the inclusion of an organic layer including the heterocyclic compound represented by Formula 1 as described above.

In one or more embodiments, in the organic light-emitting device,
the first electrode is an anode and the second electrode is a cathode,
the organic layer further includes a hole transport region between the first electrode and the emission layer and an electron transport region between the emission layer and the second electrode,
the hole transport region may include a hole injection layer, a hole transport layer, an electron blocking layer, or any combination thereof, and
the electron transport region may include a hole blocking layer, an electron transport layer, an electron injection layer, or any combination thereof, but embodiments of the present disclosure are not limited thereto.

For example, the emission layer of the organic light-emitting device may include at least one heterocyclic compound represented by Formula 1.

In one or more embodiments, the emission layer in the organic light-emitting device may include a host and a dopant, and the host may include at least one heterocyclic compound represented by Formula 1, and the dopant may include a phosphorescent dopant or a fluorescent dopant. For example, the dopant may include a phosphorescent dopant (for example, an organometallic compound represented by Formula 81). The host may further include any other host, in addition to the heterocyclic compound represented by Formula 1.

The emission layer may emit red light, green light, or blue light.

In one or more embodiments, the emission layer may include a fluorescent dopant, but embodiments of the present disclosure are not limited thereto.

In one or more embodiments, the emission layer may include a phosphorescent dopant, but embodiments of the present disclosure are not limited thereto.

In one or more embodiments, the heterocyclic compound represented by Formula 1 may be included in the hole transport region of the organic light-emitting device.

For example, the hole transport region of the organic light-emitting device includes at least one of a hole injection layer, a hole transport layer an electron blocking layer, or any combination thereof and at least one of the hole injection layer, the hole transport layer, the electron blocking layer, or any combination thereof may include the heterocyclic compound represented by Formula 1.

In one or more embodiments, the heterocyclic compound represented by Formula 1 may be included in the electron transport region of the organic light-emitting device.

For example, the electron transport region of the organic light-emitting device includes at least one of a hole blocking layer, an electron transport layer, an electron injection layer, or any combination thereof and at least one of the hole blocking layer, the electron transport layer, the electron injection layer, or any combination thereof may include the heterocyclic compound represented by Formula 1.

In one or more embodiments, the hole transport region of the organic light-emitting device may include an electron blocking layer, and the heterocyclic compound represented by Formula 1 may be included in the electron blocking layer. The electron blocking layer may be in direct contact with the emission layer.

In one or more embodiments, the electron transport region of the organic light-emitting device may include a hole blocking layer, and the heterocyclic compound represented by Formula 1 may be included in the hole blocking layer. The hole blocking layer may directly contact the emission layer.

In one or more embodiments, the organic layer of the organic light-emitting device may further include a fluorescent dopant in addition to the heterocyclic compound represented by Formula 1.

For example, the fluorescent dopant may be a condensation polycyclic compound or a styryl compound.

For example, the fluorescent dopant may include one of a naphthalene-containing core, a fluorene-containing core, a spiro-bifluorene-containing core, a benzofluorene-containing core, a dibenzofluorene-containing core, a phenanthrene-containing core, an anthracene-containing core, a fluoranthene-containing core, a triphenylene-containing core, a pyrene-containing core, a chrysene-containing core, a naphthacene-containing core, a picene-containing core, a perylene-containing core, a pentaphene-containing core, an indenoanthracene-containing core, a tetracene-containing core, a bisanthracene-containing core, a core represented by Formulae 501-1 to 501-18, or any combination thereof, but embodiments of the present disclosure are not limited thereto:

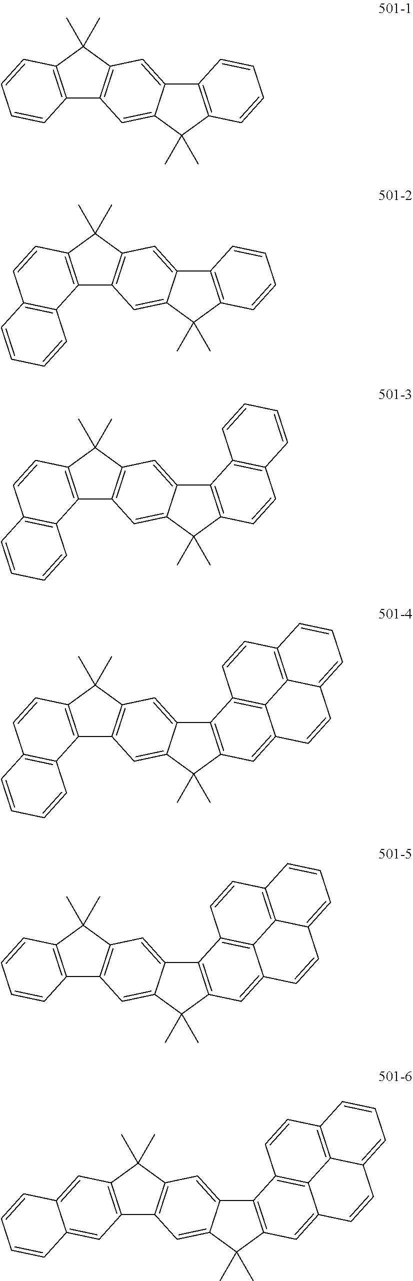

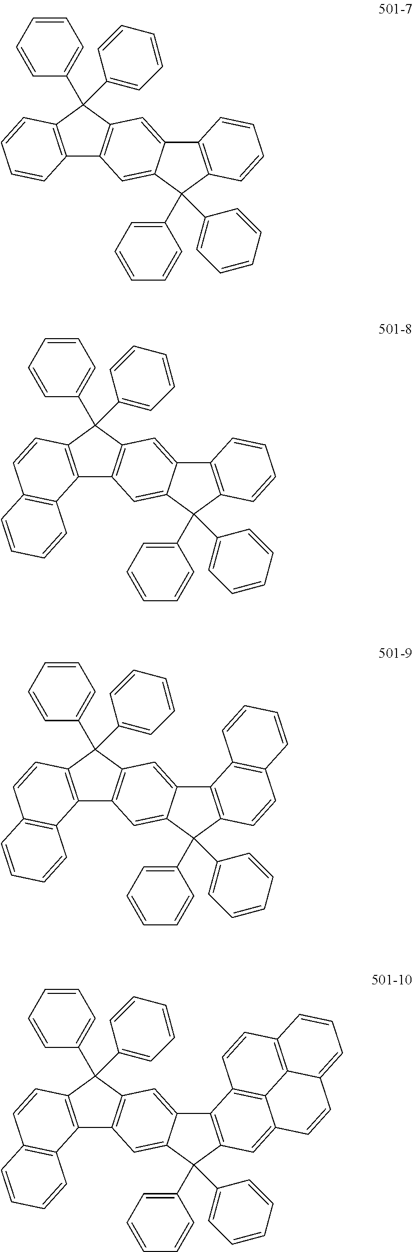

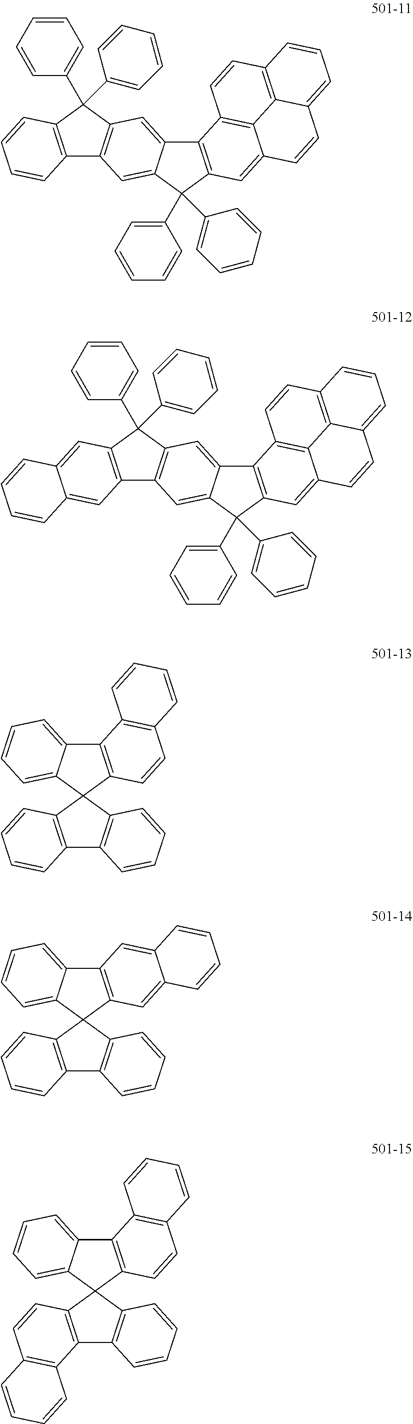

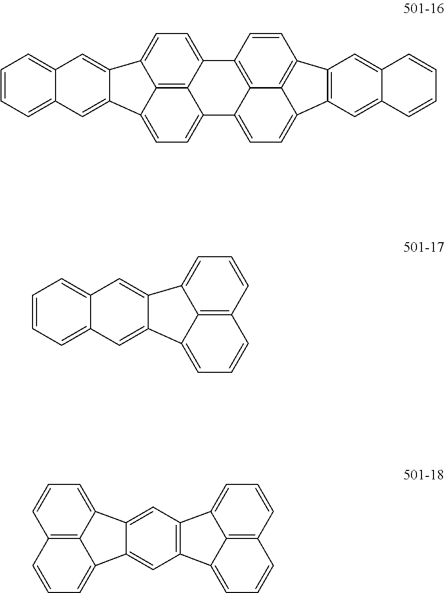

In one or more embodiments, the fluorescent dopant may be a styryl-amine-based compound or a styryl-carbazole-based compound, but embodiments of the present disclosure are not limited thereto.

In one or more embodiments, the fluorescent dopant may be a compound represented by Formula 501:

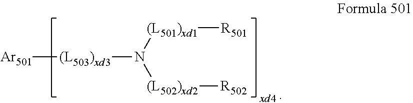

In Formula 501,
Ar₅₀₁ may be:
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by Formulae 501-1 to 501-18; or
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by Formulae 501-1 to 501-18, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group —Si(Q₅₀₁)(Q₅₀₂)(Q₅₀₃) (wherein Q₅₀₁ to Q₅₀₃ may each independently be hydrogen, C₁-C₆₀ alkyl group, a C₁-C₆₀ alkoxy group, a C₆-C₆₀ aryl group, a C₁-C₆₀ heteroaryl group, a monovalent non-aromatic condensed polycyclic group, a monovalent non-aromatic condensed heteropolycyclic group, or any combination thereof), or any combination thereof;
L₅₀₁ to L₅₀₃ may each independently be a substituted or unsubstituted C₃-C₁₀ cycloalkylene group, a substituted or unsubstituted heterocycloalkylene group, a substituted or unsubstituted C₃-C₁₀ cycloalkenylene group, a substituted or unsubstituted heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, a substituted or unsubstituted divalent non-aromatic condensed heteropolycyclic group, or any combination thereof,
R₅₀₁ and R₅₀₂ may each independently be:
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, a dibenzothiophenyl group, or any combination thereof; or
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, or any combination thereof,
xd1 to xd3 may each independently be 0, 1, 2, or 3, and
xd4 may be 0, 1, 2, 3, 4, 5, or 6.

For example, in Formula 501,
Ar₅₀₁ may be:
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by Formulae 501-1 to 501-18; or
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, a group represented by Formula 501-1 to 501-18, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a biphenyl group, a terphenyl group, a naphthyl group, a fluorenyl group, a dibenzofuranyl group, a dibenzothiophenyl group, a carbazolyl group, a pyridinyl group, a pyrimidinyl group, a triazinyl group, a quinolinyl group, an isoquinolinyl group, —Si(Q₅₀₁)(Q₅₀₂)(Q₅₀₃) (Q₅₀₁ to Q₅₀₃ may each independently be hydrogen, C₁-C₂₀ alkyl group, a C₁-C₂₀ alkoxy group, a phenyl group, a biphenyl group, a terphenyl group, a naphthyl group, or any combination thereof), or any combination thereof,
L₅₀₁ to L₅₀₃ are the same as described in connection with L₂₁,
xd1 to xd3 may each independently be 0, 1, or 2, and
xd4 may be 0, 1, 2, or 3, but embodiments of the present disclosure are not limited thereto.

In one or more embodiments, the fluorescent dopant may include a compound represented by one of Formulae 502-1 to 502-5:

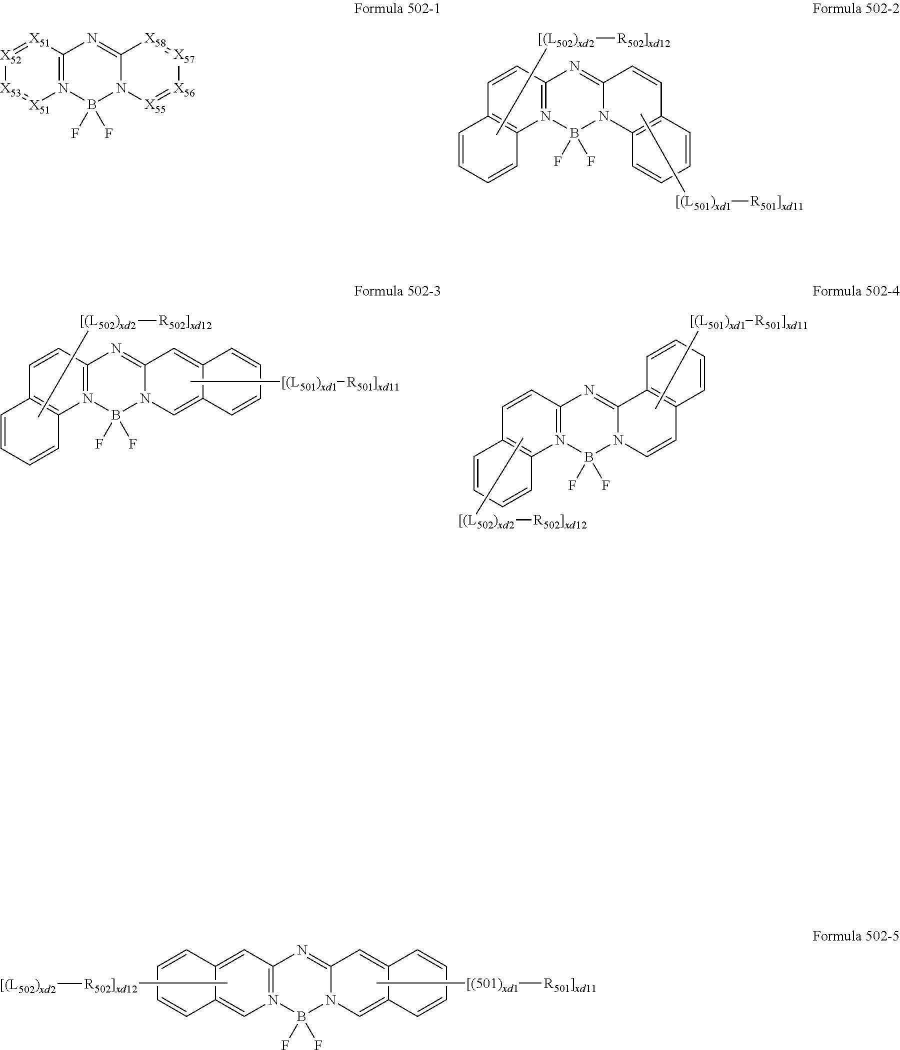

In Formulae 502-1 to 502-5,

X₅₁ may be N or C-[(L₅₀₁)_{xd1}-R₅₀₁], X₅₂ may be N or C-[(L₅₀₂)_{xd2}-R₅₀₂], X₅₃ may be N or C-[(L₅₀₃)_{xd3}-R₅₀₃], X₅₄ may be N or C-[(L₅₀₄)_{xd4}-R₅₀₄], X₅₅ may be N or C-[(L₅₀₅)_{xd5}-R₅₀₅], X₅₆ may be N or C-[(L₅₀₆)_{xd6}-R₅₀₆], X₅₇ may be N or C-[(L₅₀₇)_{xd7}-R₅₀₇], and X₅₈ may be N or C-[(L₅₀₈)_{xd8}-R₅₀₈],
L₅₀₁ to L₅₀₈ are each the same as described in connection with L₅₀₁ in Formula 501,
xd1 to xd8 are each the same as described in connection with xd1 in Formula 501,
R₅₀₁ to R₅₀₈ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, or any combination thereof,
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, a dibenzothiophenyl group, or any combination thereof; or
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, or any combination thereof,
xd11 and xd12 may each independently be an integer from 0 to 5, and
two of R₅₀₁ to R₅₀₄ may optionally be linked together to form a saturated or unsaturated ring, and
two of R₅₀₅ to R₅₀₅ may optionally be linked together to form a saturated or unsaturated ring.

The fluorescent dopant may include at least one compound, for example, the following compounds FD(1) to FD(16) and FD1 to FD14:

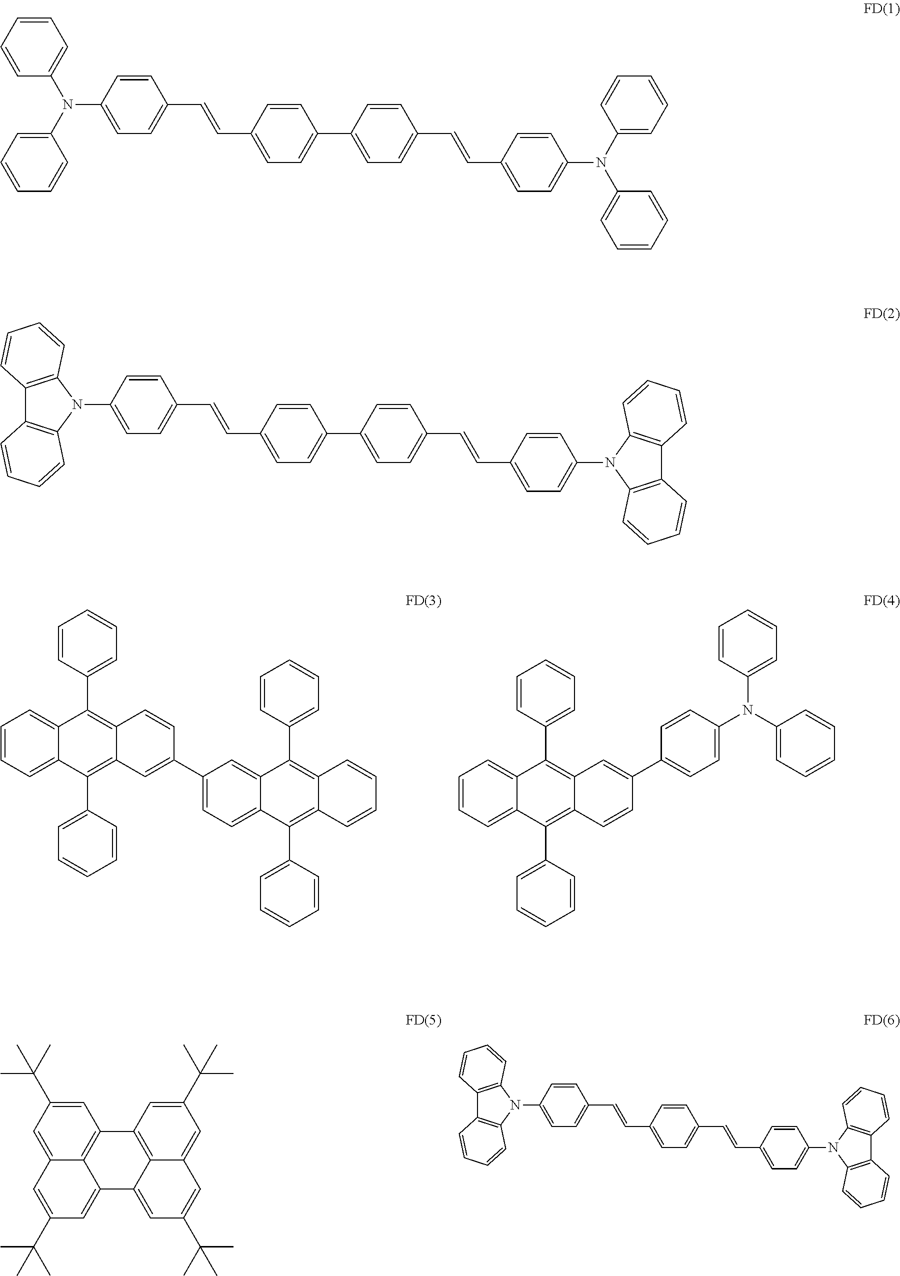

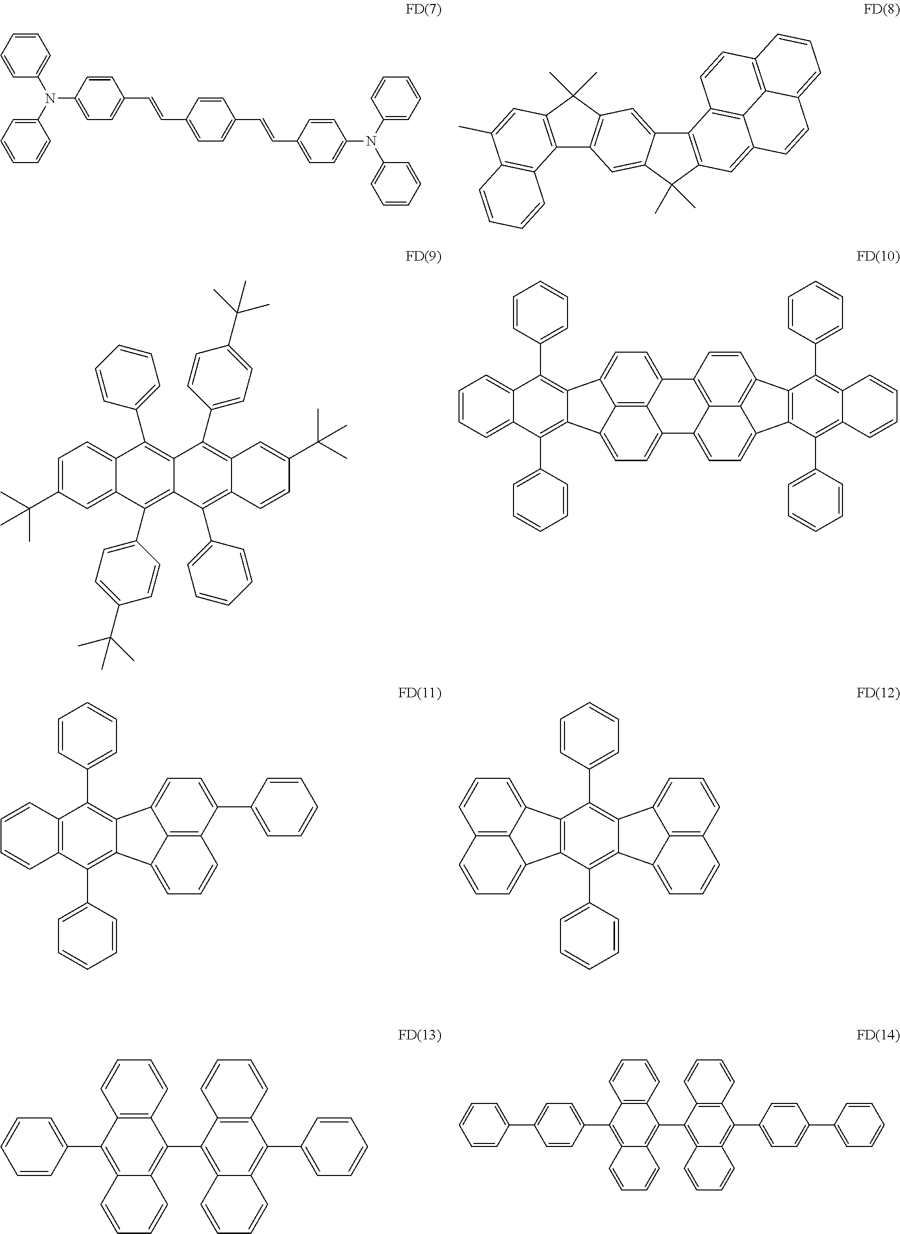

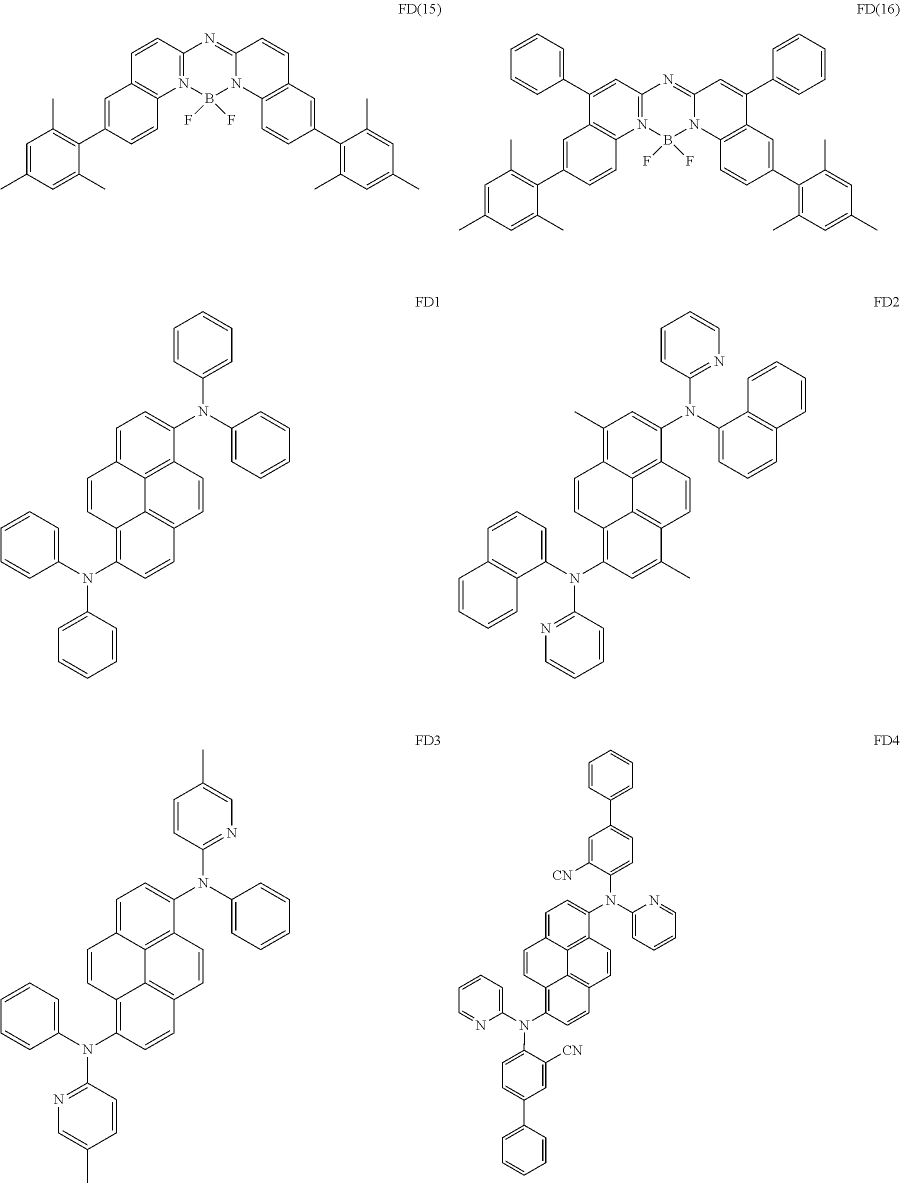

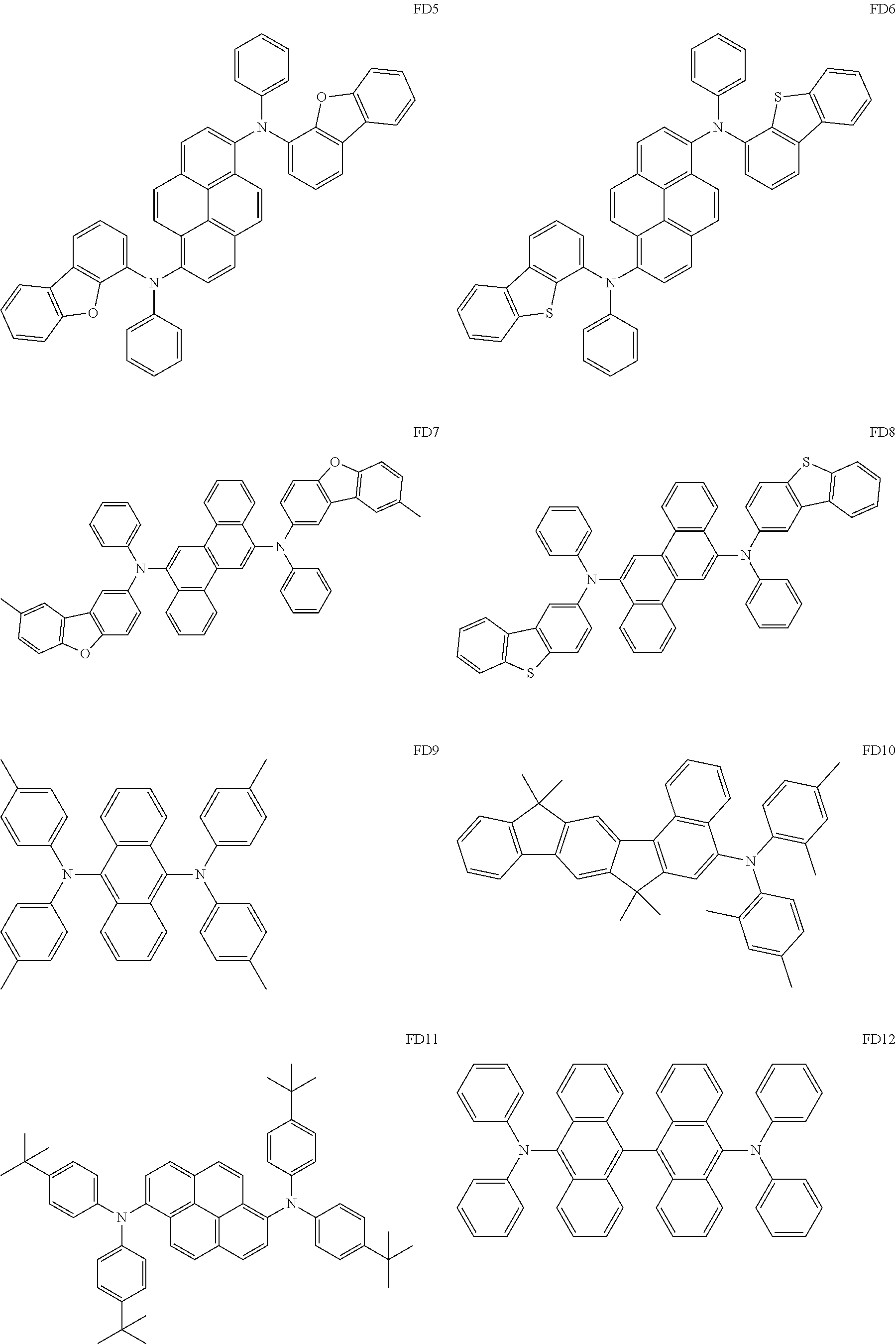

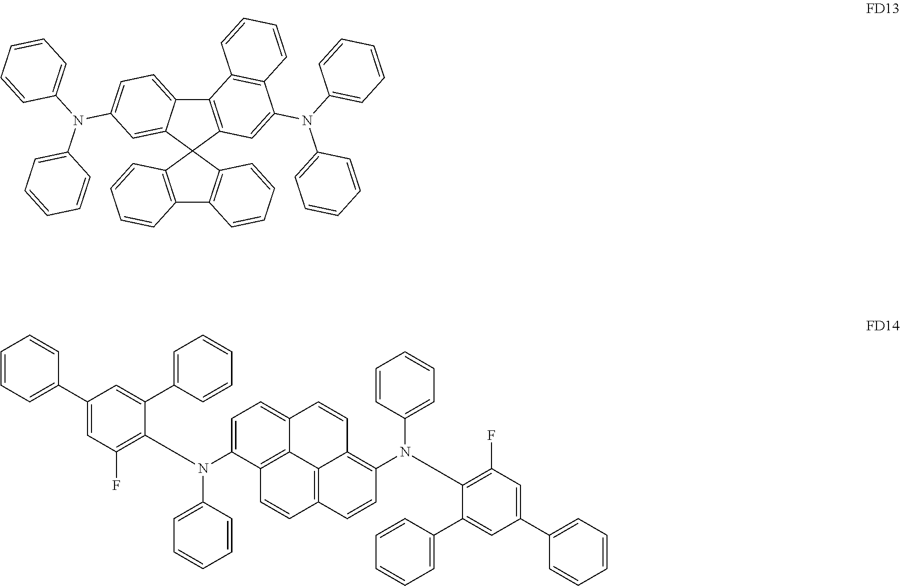 [SMILES: c1ccc(N(c2ccccc2)c2ccc3c(c2)C2(c4ccccc4-c4ccccc42)c2cc(N(c4ccccc4)c4ccccc4)c4ccccc4c2-3)cc1 ; Fc1cc(-c2ccccc2)cc(-c2ccccc2)c1N(c1ccccc1)c1ccc2ccc3c(N(c4ccccc4)c4c(F)cc(-c5ccccc5)cc4-c4ccccc4)ccc4ccc1c2c43]

In one or more embodiments, the organic layer of the organic light-emitting device may further include a phosphorescent dopant in addition to the heterocyclic compound represented by Formula 1.

For example, the phosphorescent dopant may further include an organometallic compound represented by Formula 81:

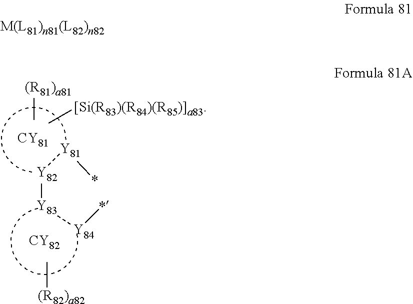

In Formulae 81 and 81A,
M may be iridium (Ir), platinum (Pt), osmium (Os), titanium (Ti), zirconium (Zr), hafnium (Hf), europium (Eu), terbium (Tb), thulium (Tm), or rhodium (Rh),
L₈₁ may be a ligand represented by Formula 81A, n81 may be an integer from 1 to 3, and when n81 is 2 or more, two or more of L₈₁(s) may be identical to or different from each other,
L₈₂ may be an organic ligand, n82 may be an integer from 0 to 4, and when n82 is 2 or more, two or more of L₅₂(s) may be identical to or different from each other,
Y₈₁ to Y₈₄ may each independently be carbon (C) or nitrogen (N),
Y₈₁ and Y₈₂ may be linked to each other via a single bond or a double bond, and Y₈₃ and Y₈₄ may be linked to each other via a single bond or a double bond,
CY₈₁ and CY₈₂ may each independently be a C₅-C₃₀ carbocyclic group, a C₂-C₃₀ heterocarbocyclic group, or any combination thereof,
additionally, CY₈₁ and CY₈₂ may optionally be linked to each other via an organic linking group,
R₈₁ to R₈₅ may each independently be hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, —SF₅, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-Cis heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₈₁)(Q₈₂)(Q₈₃), —N(Q₈₄)(Q₈₅), —B(Q₈₆)(Q₈₇), —P(═O)(Q₈₈)(Q₈₉), or any combination thereof,
a81 to a83 may each independently be an integer from 0 to 5,
when a81 is two or more, two or more R₈₁(s) may be identical to or different from each other,
when a82 is two or more, two or more Ru(s) may be identical to or different from each other,
when a81 is 2 or more, neighboring two R₈₁(s) may optionally be linked to form a saturated or unsaturated 02-030 ring (for example, a benzene ring, a cyclopentane ring, a cyclohexane ring, a cyclopentene ring, a cyclohexene ring, a norbornane ring, a bicyclo[2.2.1]heptane ring, a naphthalene ring, a benzoindene ring, a benzoindole ring, a benzofuran ring, a benzothiophene ring, a pyridine ring, a pyrimidine ring, or a pyrazine ring) or a saturated or unsaturated 02-030 ring substituted with at least one R₈₈(for example, a benzene ring, cyclopentane ring, a cyclohexane ring, cyclopentene ring, a cyclohexene ring, norbornane ring, a bicyclo[2.2.1]heptane ring, a naphthalene ring, benzoindene ring, benzoindole ring, a benzofuran ring, a benzothiophene ring, a pyridine ring, a pyrimidine ring, or a pyrazine ring, each substituted with at least one R₈₈),
when a82 is 2 or more, neighboring two R₈₂(s) may optionally be linked to form a saturated or unsaturated C₂-C₃₀ ring (for example, a benzene ring, a cyclopentane ring, a cyclohexane ring, a cyclopentene ring, a cyclohexene ring, a norbornane ring, a bicyclo[2.2.1]heptane ring, a naphthalene ring, a benzoindene ring, a benzoindole ring, a benzofuran ring, a benzothiophene ring, a pyridine ring, a pyrimidine ring, or a pyrazine ring) or a saturated or unsaturated C₂-C₃₀ ring substituted with at least one R₈₉ (for example, a benzene ring, cyclopentane ring, a cyclohexane ring, cyclopentene ring, a cyclohexene ring, norbornane ring, a bicyclo[2.2.1]heptane ring, a naphthalene ring, benzoindene ring, benzoindole ring, a benzofuran ring, a benzothiophene ring, a pyridine ring, a pyrimidine ring, or a pyrazine ring, each substituted with at least one R₈₉),
R₈₈ may be the same as explained in connection with R₈₁,
R₈₉ may be the same as explained in connection with R₈₂,
* and *′ in Formula 81A each indicates a binding site to M in Formula 81, and
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₈-C₈₀ aryl group, the substituted C₈-C₈₀ aryloxy group, the substituted C₈-C₈₀ arylthio group, the substituted C₁-C₆₀ heteroaryl group, the substituted monovalent non-aromatic condensed polycyclic group, and substituted monovalent non-aromatic condensed heteropolycyclic group may be deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₉₁)(Q₉₂)(Q₉₃), or any combination thereof,
wherein Q₈₁ to Q₈₉ and Q₉₁ to Q₉₃ may each independently be hydrogen, deuterium, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, or any combination thereof.

In one or more embodiments, in Formula 81A,
a83 may be 1 or 2,
R₈₃ to R₈₅ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CH₃, —CD₂CD₃, —CD₂CD₂H, —CD₂CDH₂, or any combination thereof;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, or any combination thereof; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, or any combination thereof, each substituted with at least one deuterium, a C₁-C₁₀ alkyl group, a phenyl group, or any combination thereof, but embodiments of the present disclosure are not limited thereto.

In one or more embodiments, in Formula 81A,
Y₈₁ may be nitrogen, Y₈₂ and Y₈₃ may be carbon, Y₈₄ may be nitrogen or carbon,
CY₈₁ and CY₈₂ may each independently be a cyclopentadiene group, a benzene group, a heptalene group, an indene group, a naphthalene group, an azulene group, a heptalene group, an indacene group, acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, an indazole group, a pyrazole group, an imidazole group, a triazole group, an oxazole group, an isoxazole group, an oxadiazole group, a thiazole group, an isothiazole group, a thiadiazole group, a purine group, a furan group, a thiophene group, a pyridine group, a pyrimidine group, a quinoline group, an isoquinoline group, a benzoquinoline group, a phthalazine group, a naphthyridine group, a quinoxaline group, a quinazoline group, a cinnoline group, a phenanthrididine group, an acridine group, a phenanthroline group, a phenazine group, a benzimidazole group, a benzofuran group, a benzothiophene group, an isobenzothiazole group, a benzoxazole group, an isobenzoxazole group, a benzocarbazole group, a dibenzocarbazole group, an imidazopyridine group, an imidazopyrimidine group, a dibenzofuran group, a dibenzothiophene group, a dibenzothiophene sulfone group, a carbazole group, a dibenzosilole group, a 2,3-dihydro-1H-imidazole group, or any combination thereof.

In one or more embodiments, in Formula 81A, Y₈₁ may be nitrogen, Y₈₂ to Y₈₄ may each be carbon, CY₈₁ may be 5-membered rings in which two nitrogen atoms are ring-forming atoms, and CY₈₂ may be a benzene group, a naphthalene group, a fluorene group, a dibenzofuran group, or a dibenzothiophene group, but embodiments of the present disclosure are not limited thereto.

In one or more embodiments, in Formula 81A, Y₈₁ may be nitrogen, Y₈₂ to Y₈₄ may each be carbon, CY₅₁ may be an imidazole group or a 2,3-dihydro-1H-imidazole group, and CY₅₂ may be a benzene group, a naphthalene group, a fluorene group, a dibenzofuran group, or a dibenzothiophene group, but embodiments of the present disclosure are not limited thereto.

In one or more embodiments, in Formula 81A,
Y₈₁ may be nitrogen and Y₈₂ to Y₈₄ may be carbon,
CY₅₁ may be a pyrrole group, a pyrazole group, an imidazole group, a triazole group, an oxazole group, an isoxazole group, an oxadiazole group, a thiazole group, an isothiazole group, a thiadiazole group, a pyridine group, a pyrimidine group, a quinoline group, an isoquinoline group, a benzoquinoline group, a phthalazine group, a naphthyridine group, a quinoxaline group, a quinazoline group, a cinnoline group, a benzimidazole group, an isobenzothiazole group, a benzoxazole group, or an isobenzoxazole group, and
CY₅₂ may be a cyclopentadiene group, a benzene group, a naphthalene group, a fluorene group, a benzofluorene group, a dibenzofluorene group, a phenanthrene group, an anthracene group, a triphenylene group, a pyrene group, a chrysene group, a perylene group, a benzofuran group, a benzothiophene group, a benzocarbazole group, a dibenzocarbazole group, a dibenzofuran group, a dibenzothiophene group, a dibenzothiophene sulfone group, a carbazole group, or a dibenzosilole group.

In one or more embodiments, in Formula 81A
R₈₁ and R₈₂ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, C₁-C₂₀ alkyl group, a C₁-C₂₀ alkoxy group, or any combination thereof;
a C₁-C₂₀ alkyl group, a C₁-C₂₀ alkoxy group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₁₀ alkyl group, a cyclopentyl group, a cyclohexyl group, a cycloheptyl group, a cyclooctyl group, an adamantanyl group, a norbomanyl group, a norbornenyl group, a cyclopentenyl group, a cydohexenyl group, a cycloheptenyl group, a phenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbomanyl group, a norbornenyl group, a cyclopentenyl group, a cydohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof;
a cyclopentyl group, a cyclohexyl group, a cycloheptyl group, a cyclooctyl group, an adamantanyl group, a norbomanyl group, a norbornenyl group, a cyclopentenyl group, a cydohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof; or
—B(Q₈₆)(Q₈₇), or —P(═O)(Q₈₈)(Q₈₉), or any combination thereof,
wherein Q₈₆ to Q₈₉ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CH₃, —CD₂CD₃, —CD₂CD₂H, —CD₂CDH₂, or any combination thereof;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, or any combination thereof; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, or any combination thereof, each substituted with at least one deuterium, a C₁ to C₁₀ alkyl group, a phenyl group, or any combination thereof.

In one or more embodiments, in Formula 81A, at least one R₈₁(s) in the number of a81 and R₈₂(s) in the number of a82 may be a cyano group.

In one or more embodiments, in Formula 81A, at least one R₈₂(s) in the number of a82 may be a cyano group.

In one or more embodiments, in Formula 81A, at least one R₈₁(s) in the number of a81 and R₈₂(s) in the number of a82 may be a deuterium.

In one or more embodiments, L₈₂ in Formula 81 may be a ligand represented by one of Formulae 3-1(1) to 3-1(69), 3-1(71) to 3-1(79), 3-1(81) to 3-1(88), 3-1(91) to 3-1(98), and 3-1(101) to 3-1(114):

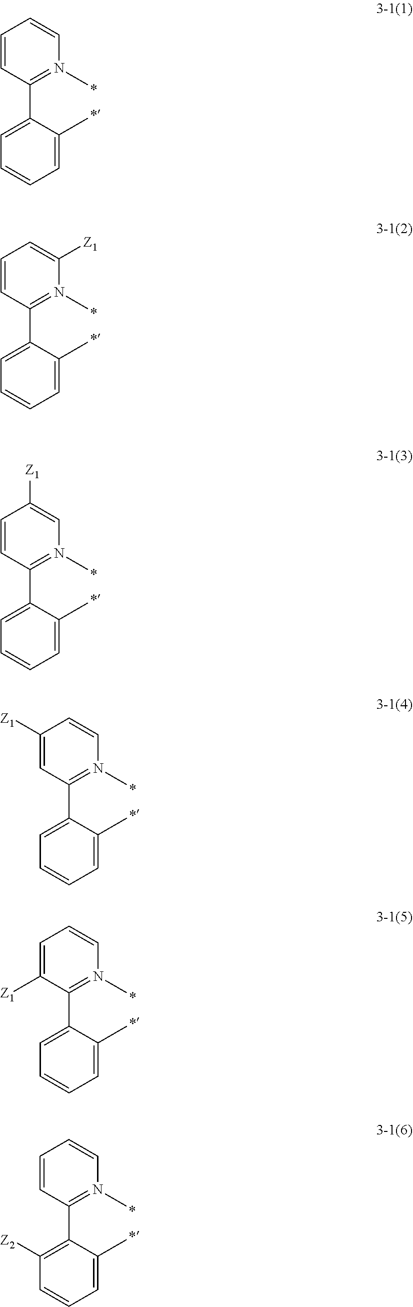

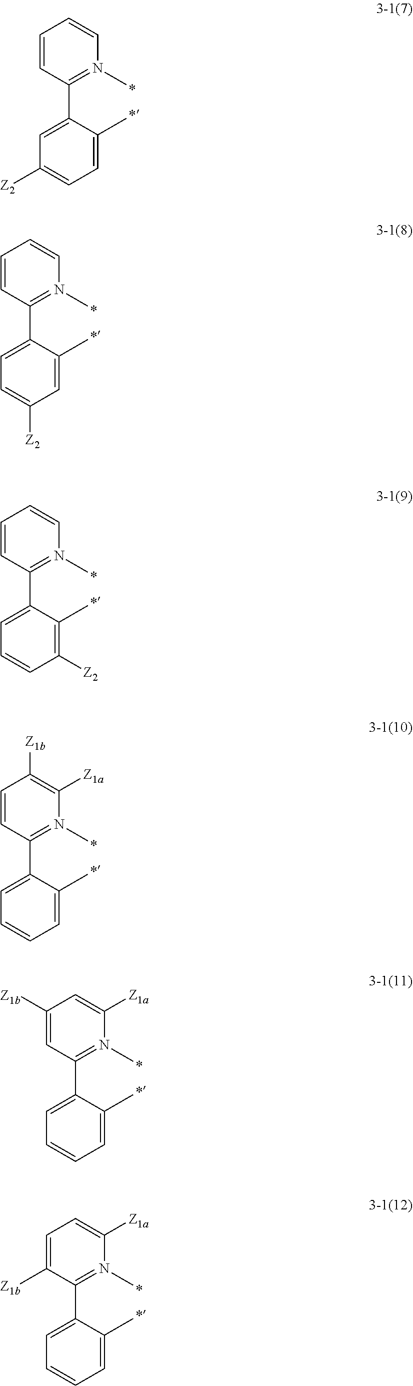

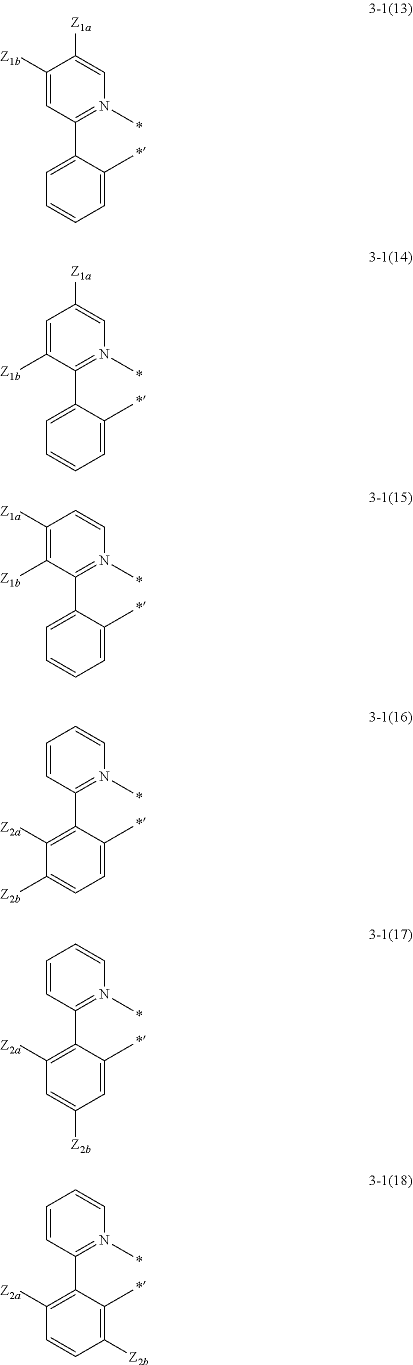

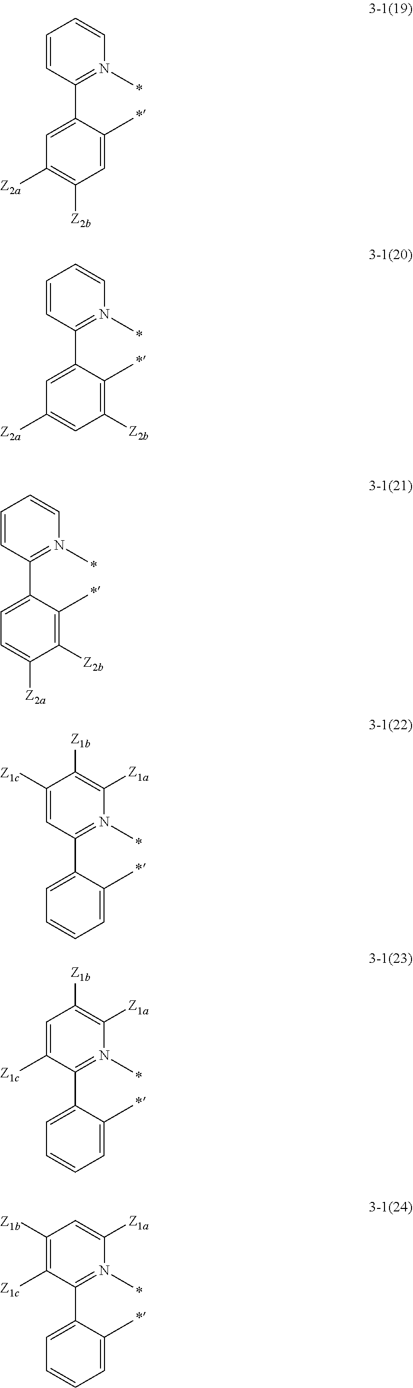

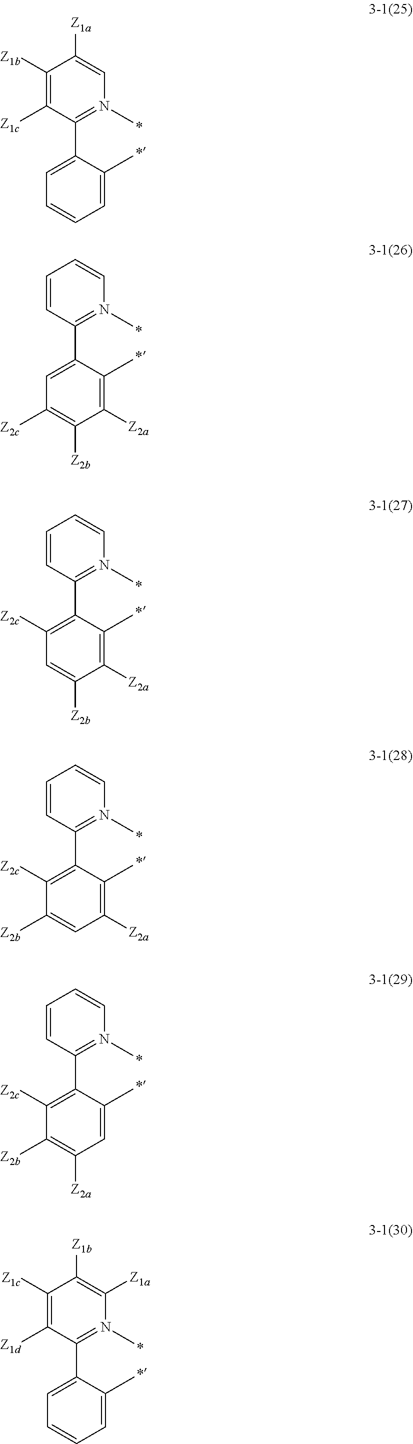

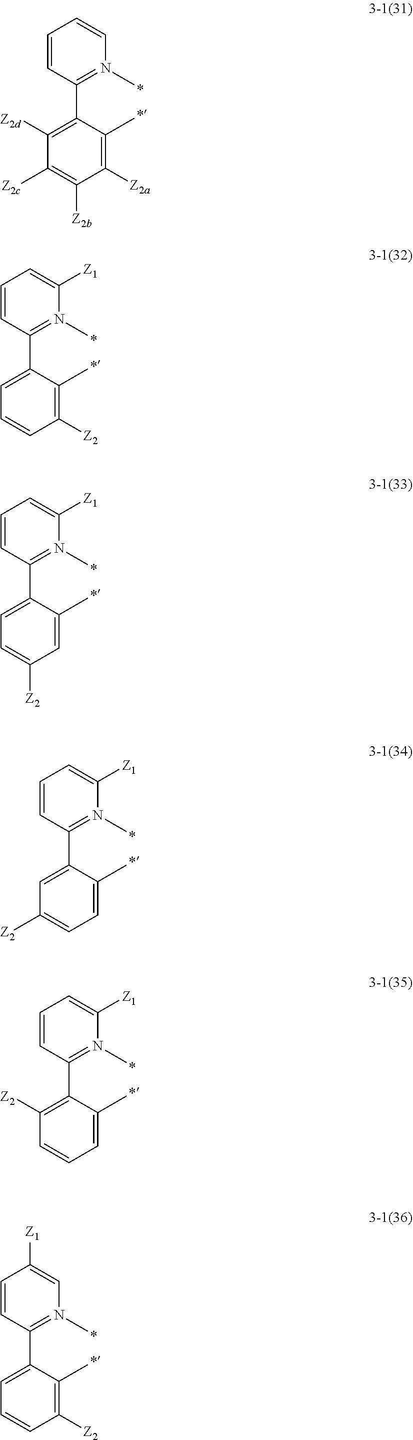

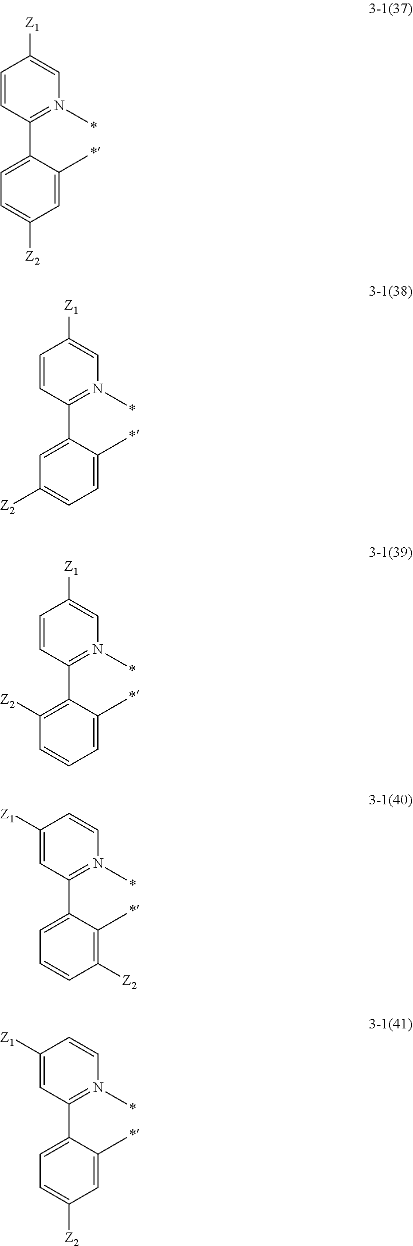

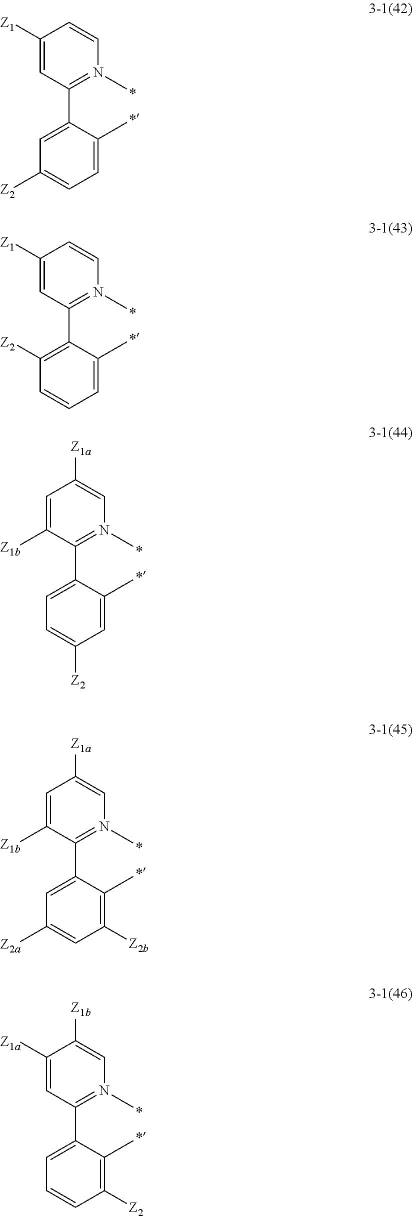

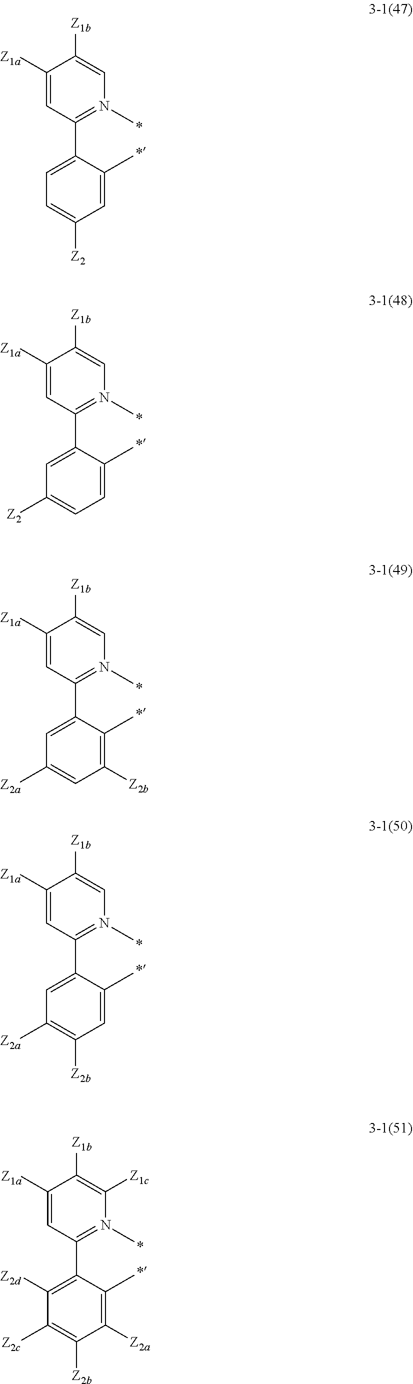

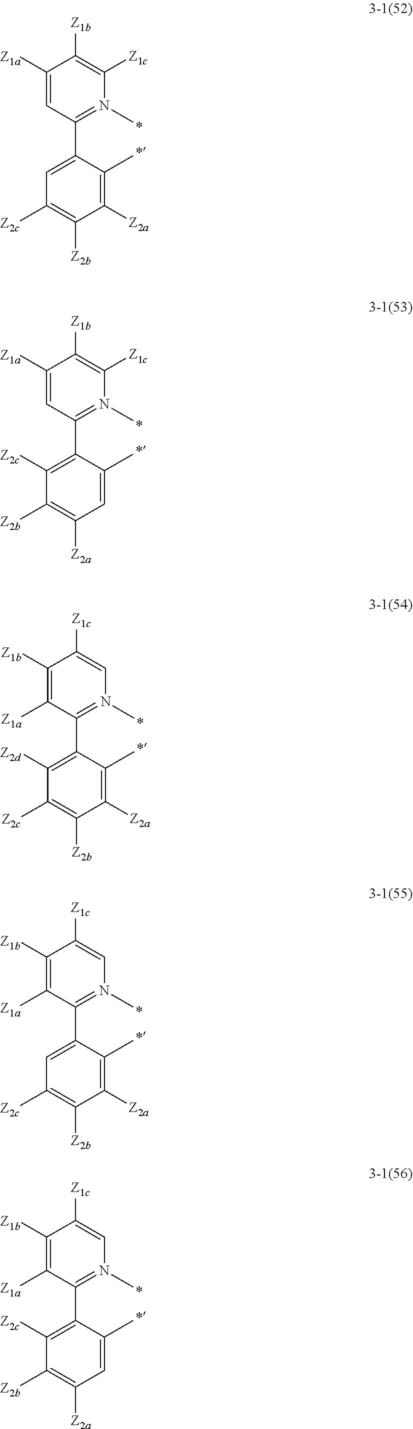

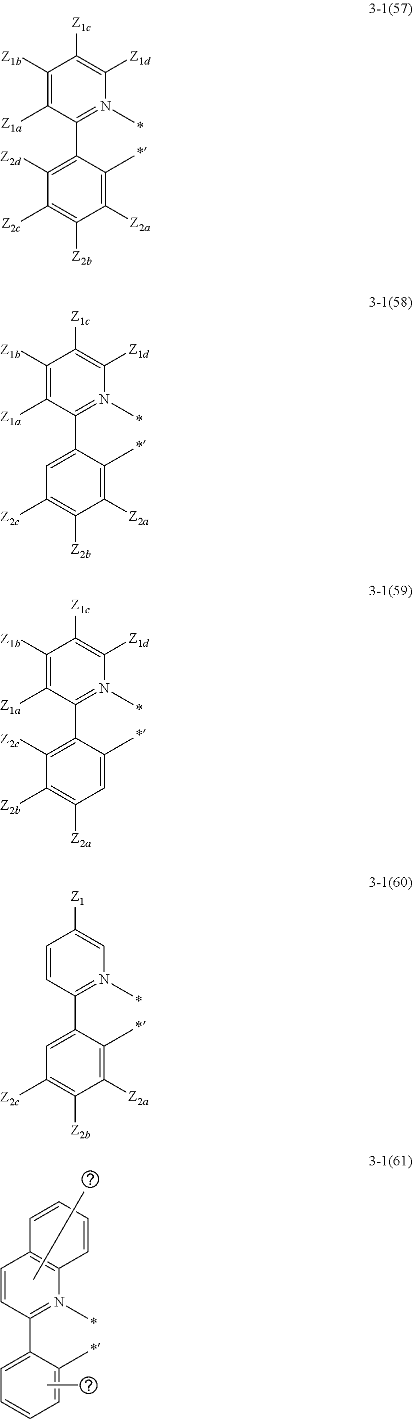

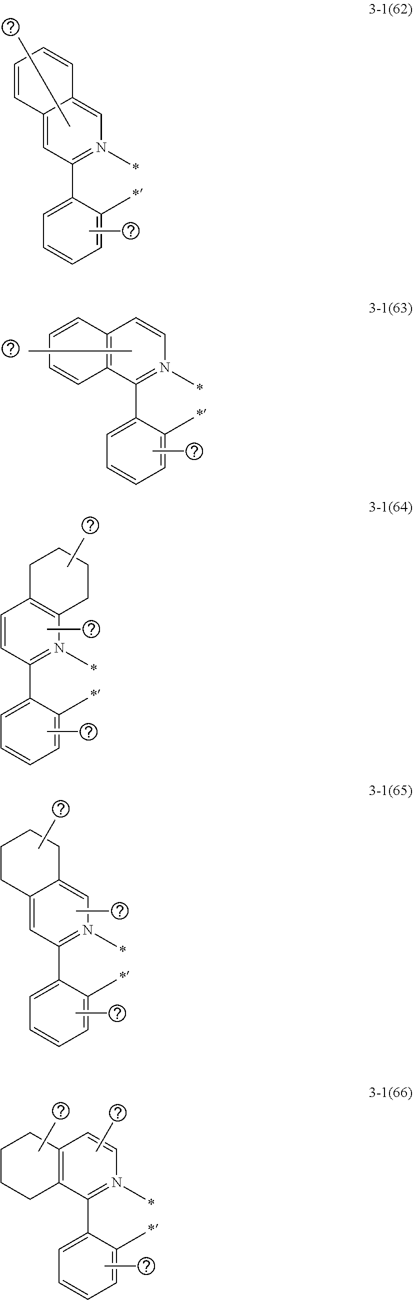

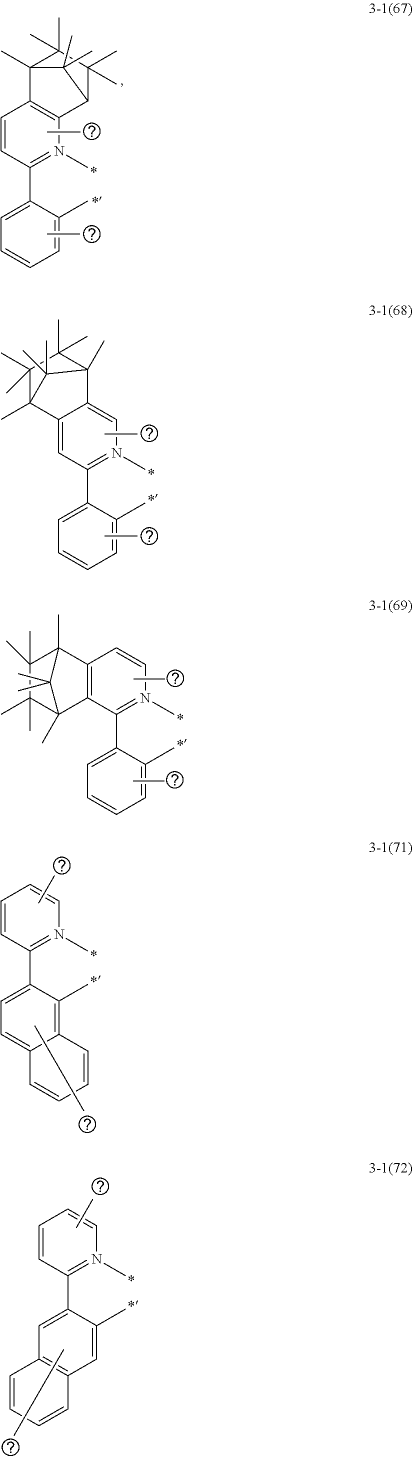

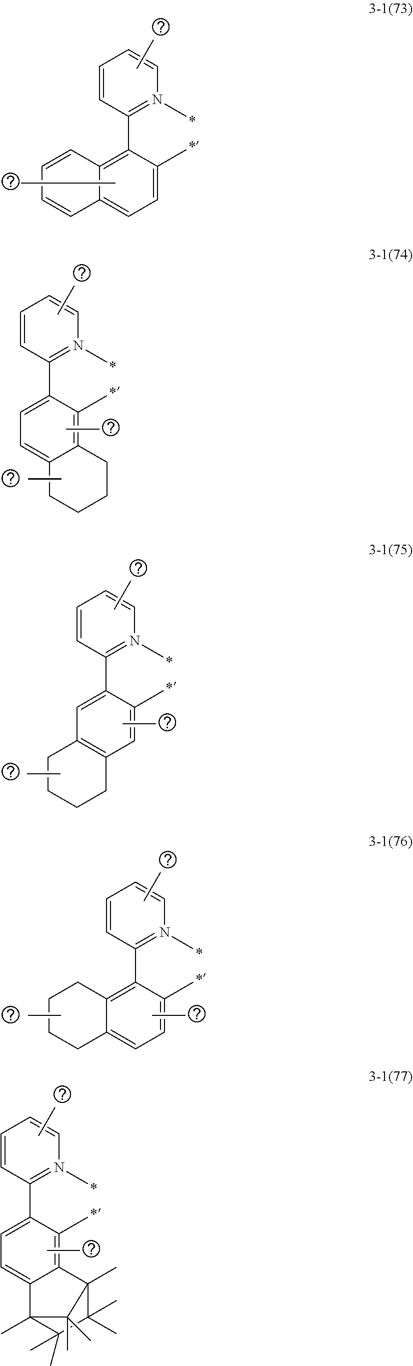

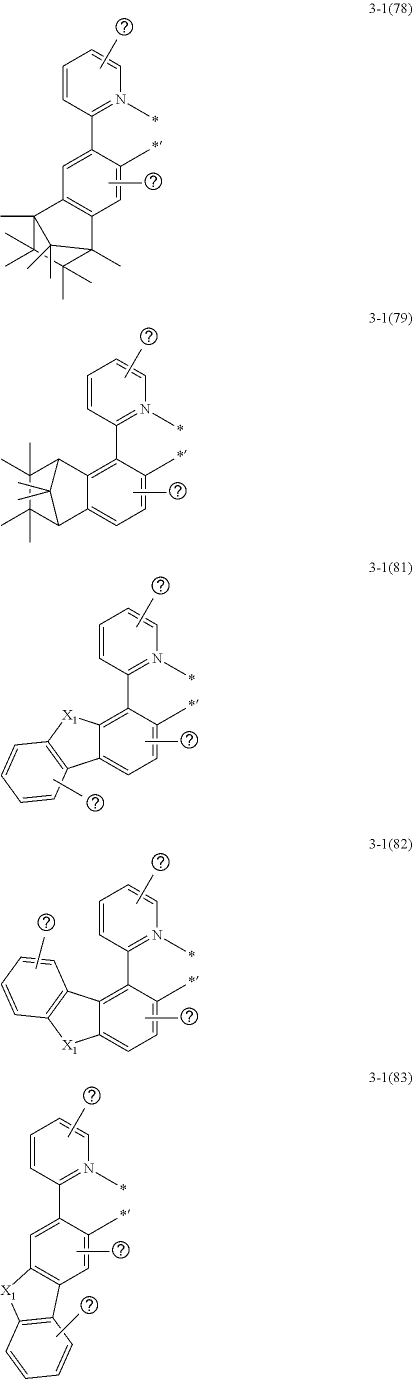

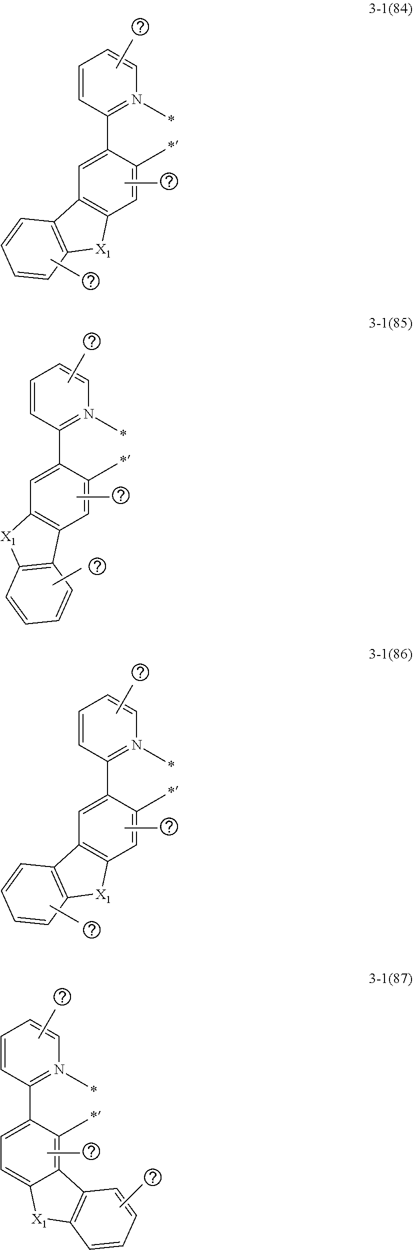

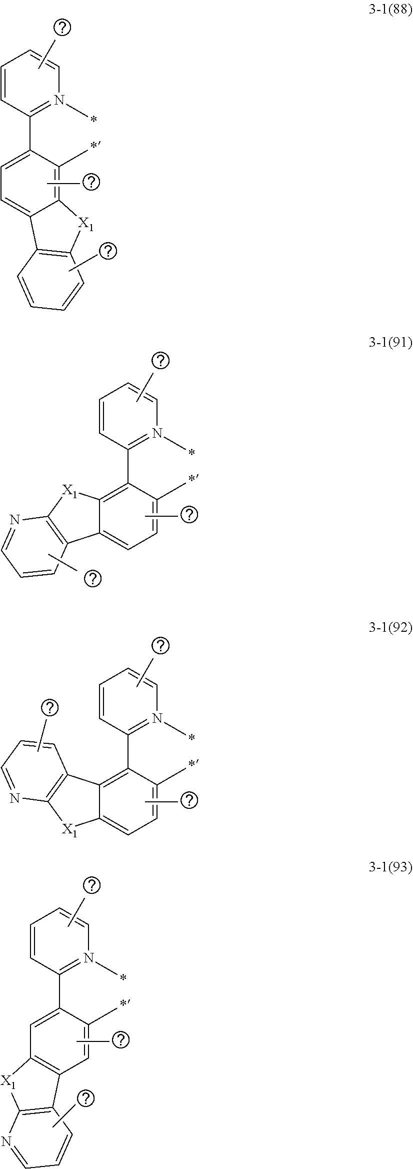

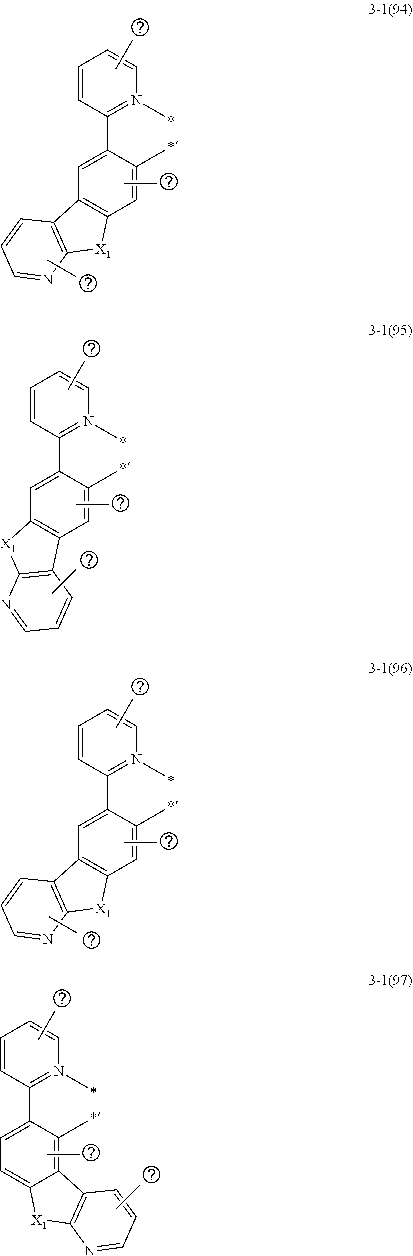

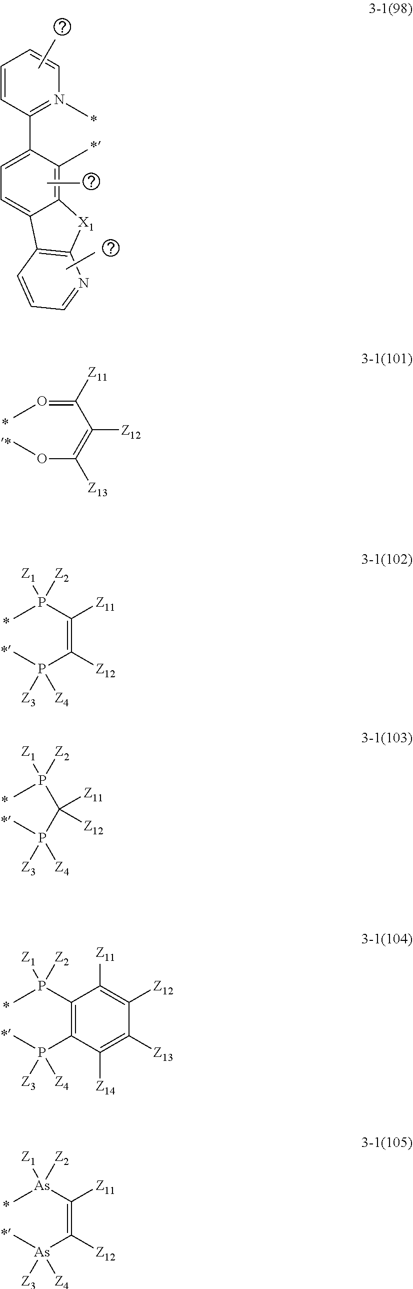

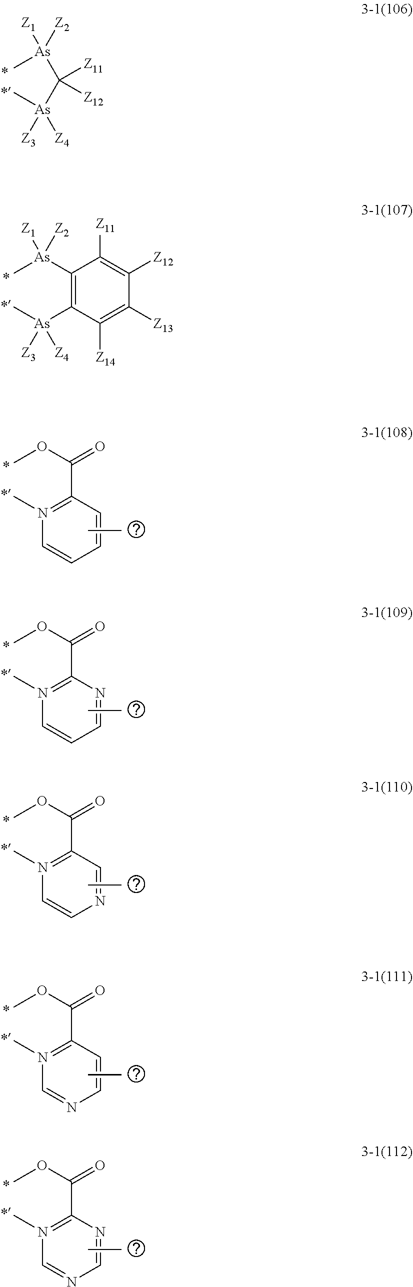

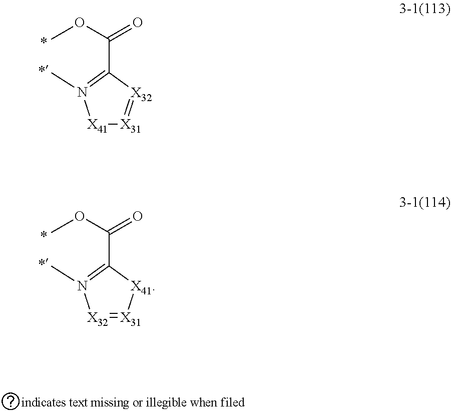

In Formulae 3-1(1) to 3-1(69), 3-1(71) to 3-1(79), 3-1(81) to 3-1(88), 3-1(91) to 3-1(98), and 3-1(101) to 3-1(114),
X₁ may be O, S, C(Z₂₁)(Z₂₂), or N(Z₂₃),
X₃₁ may be N or C(Zia) and X₃₂ may be N or C(Z_{1b}),
X₄₁ may be O, S, N(Z₁ₐ), or C(Z₁ₐ)(Z_{1b}),
Z₁ to Z₄, Z₁ₐ, Z_{1b}, Z_{1c}, Z_{1d}, Z₂ₐ, Z_{2b}, Z_{2c}, Z_{2d}, Z₁₁ to Z₁₄ and Z₂₁ to Z₂₃ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, C₁-C₂₀ alkyl group, a C₁-C₂₀ alkoxy group, or any combination thereof;
a C₁-C₂₀ alkyl group, a C₁-C₂₀ alkoxy group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof; or
—B(Q₈₆)(Q₈₇), —P(═O)(Q₈₈)(Q₈₉), or any combination thereof,
wherein Q₈₆ to Q₈₉ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CH₃, —CD₂CD₃, —CD₂CD₂H, —CD₂CDH₂, or any combination thereof;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, or any combination thereof; or an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, and a naphthyl group, each substituted with at least one deuterium, a C₁ to C₁₀ alkyl group, a phenyl group, or any combination thereof;
d2 and e2 may each independently be 0 or 2,
e3 may be an integer from 0 to 3,
d4 and e4 may each independently be an integer from 0 to 4,
d6 and e6 may each independently be an integer from 0 to 6,
d8 and e8 may each independently be an integer from 0 to 8,
* and *′ each indicate a binding site to M in Formula 1.

In one or more embodiments, in Formula 81, M may be Ir and the sum of n81 and n82 may be 3; or M may be Pt and the sum of n81 and n82 may be 2.

In one or more embodiments, the organometallic compound represented by Formula 81 may be electrically neutral rather than a salt consisting of the pair of a cation and an anion.

In one or more embodiments, the organometallic compound represented by Formula 81 may include at least one compound of PD1 to PD78 and FIr₆, but embodiments of the present disclosure are not limited thereto.

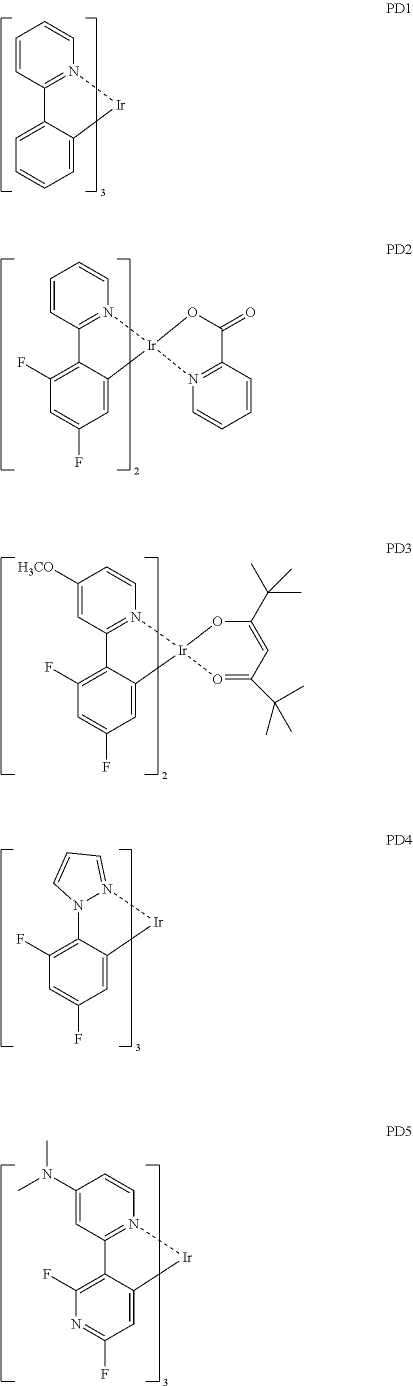

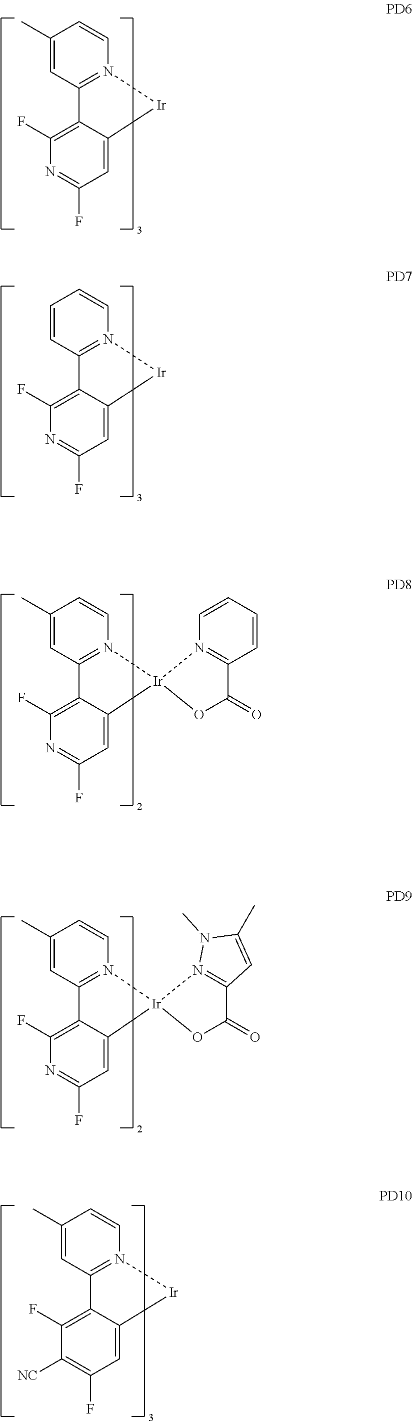

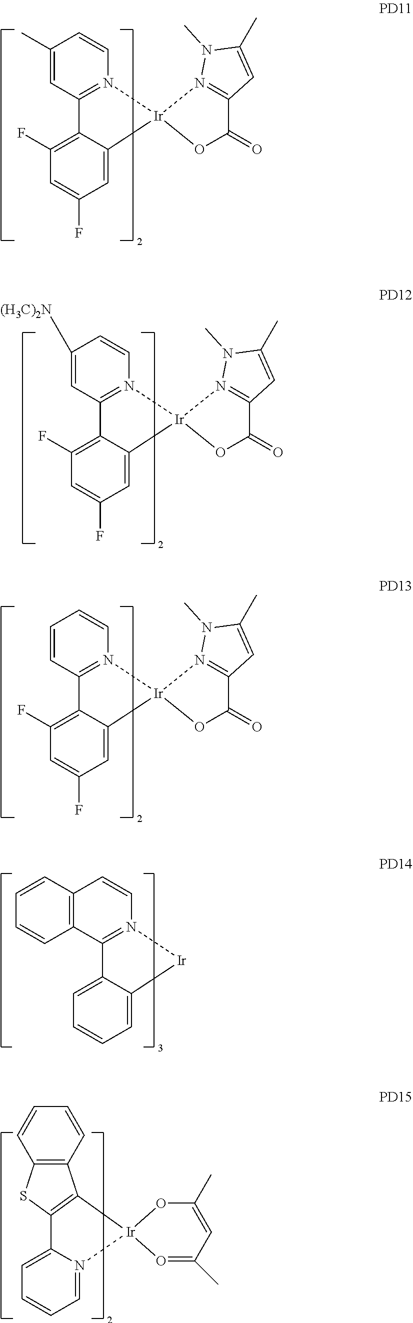

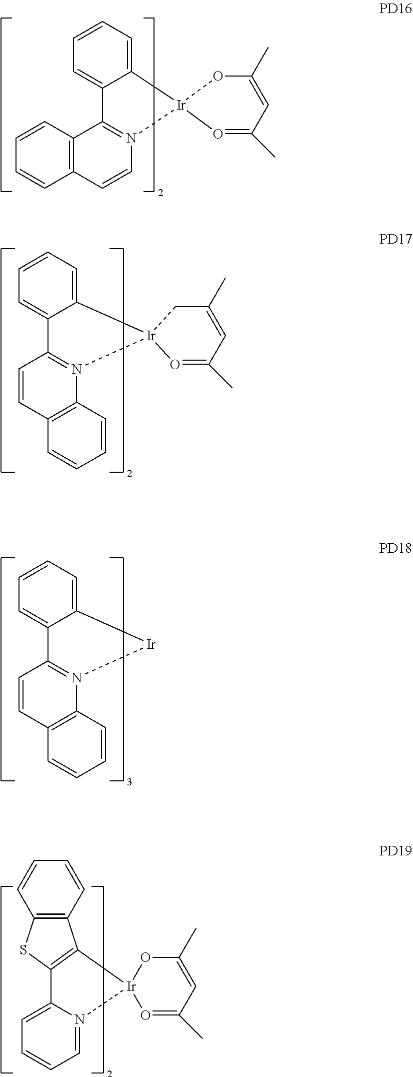

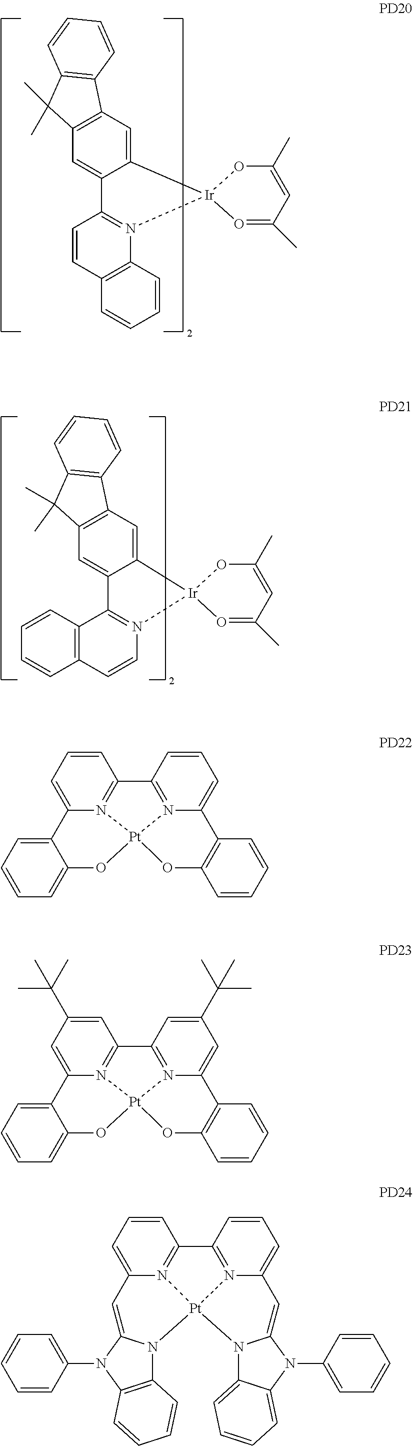

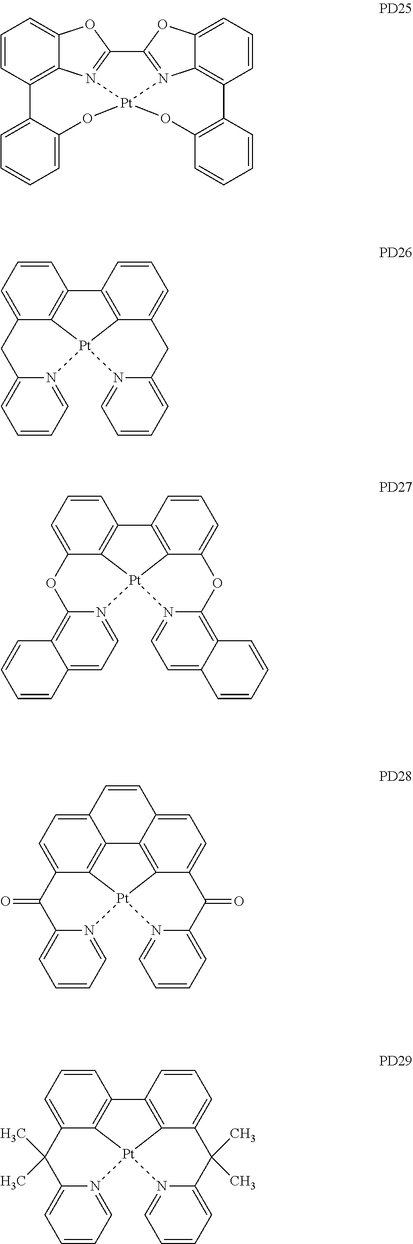

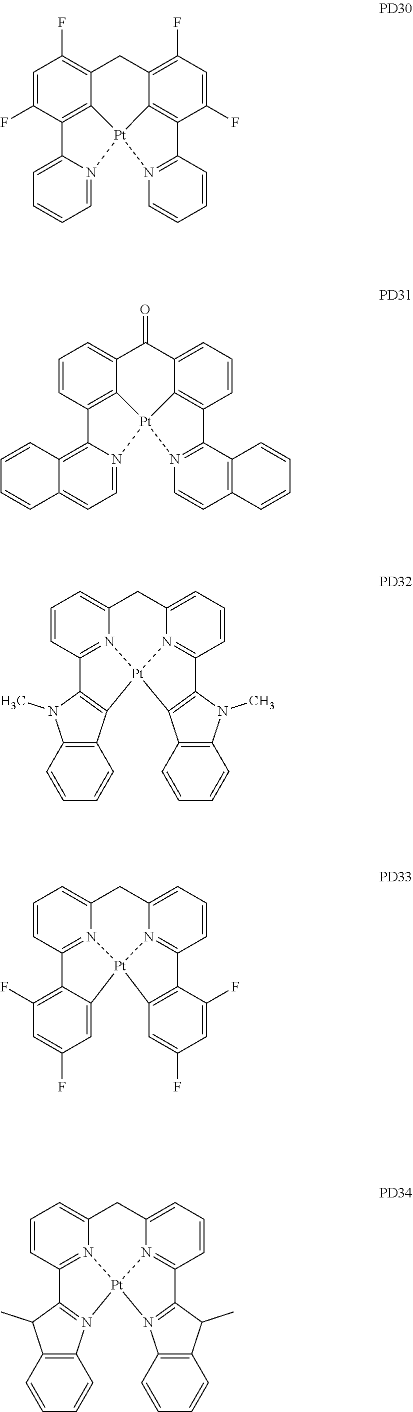

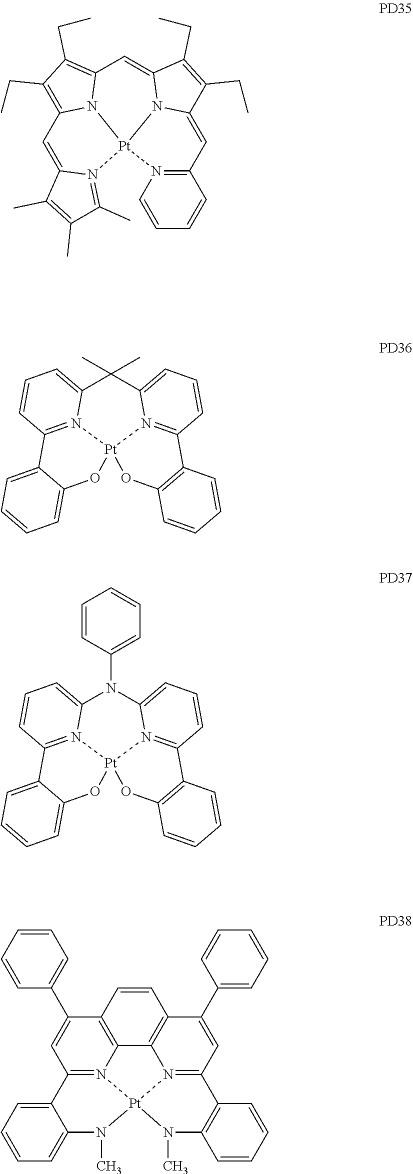

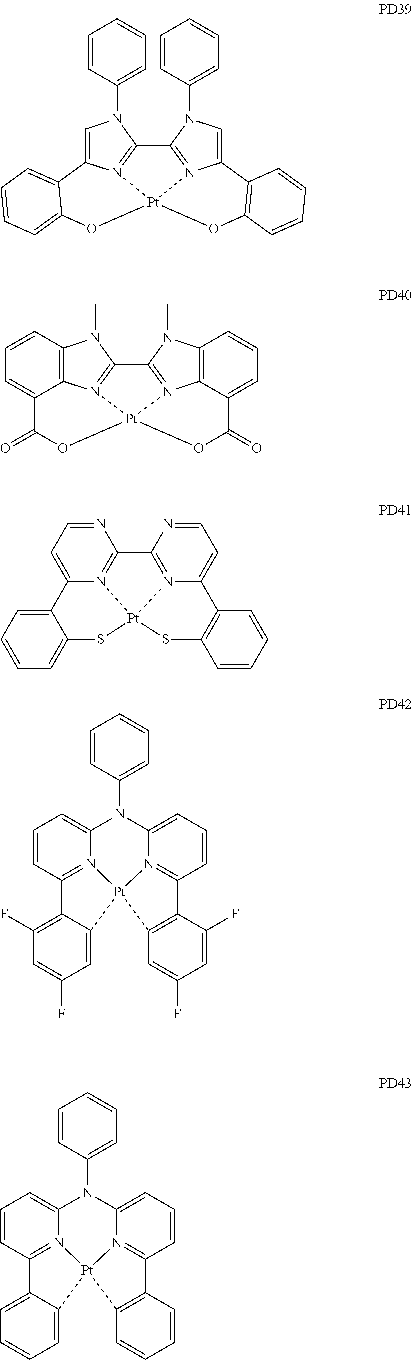

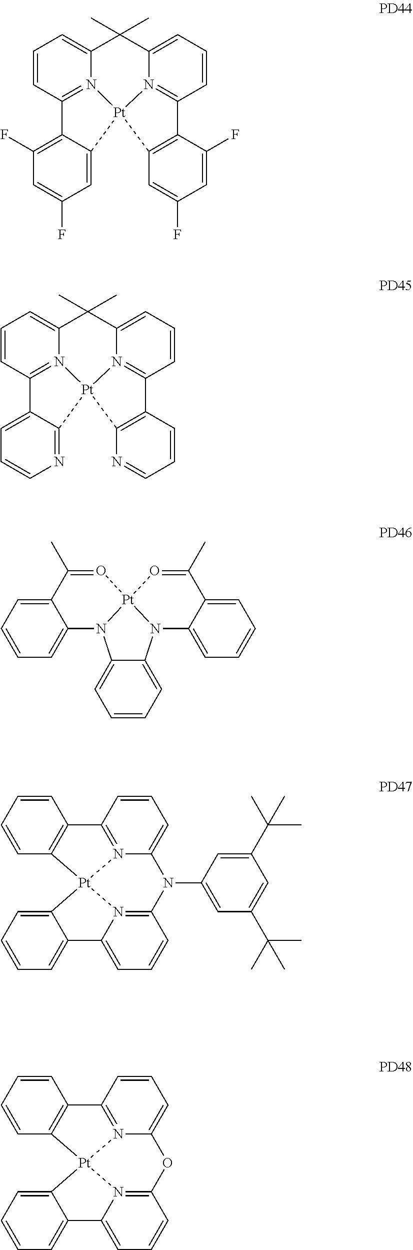

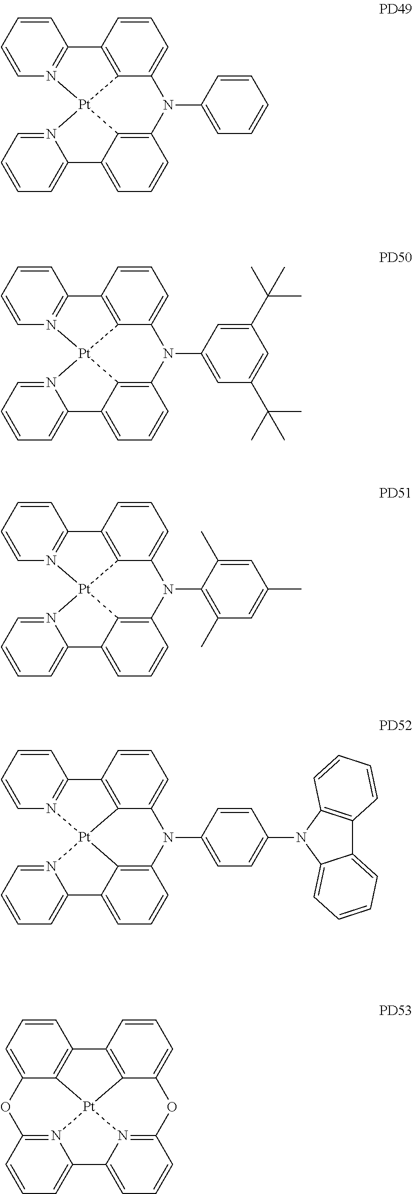

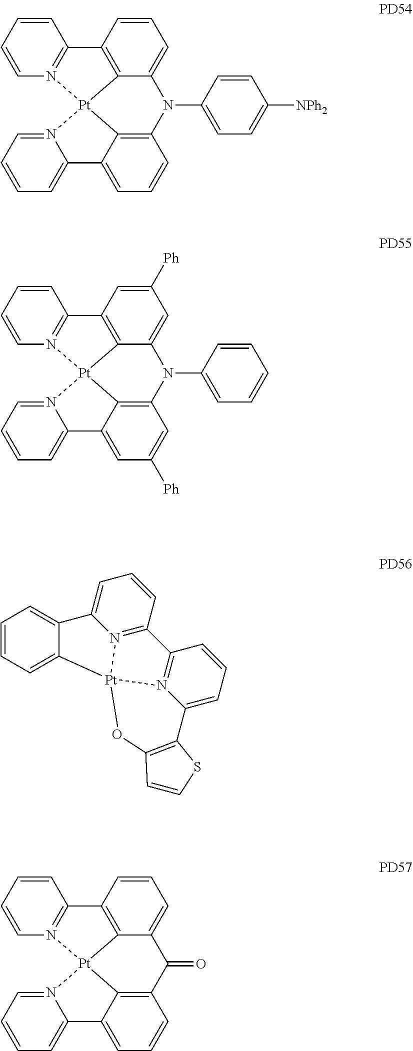

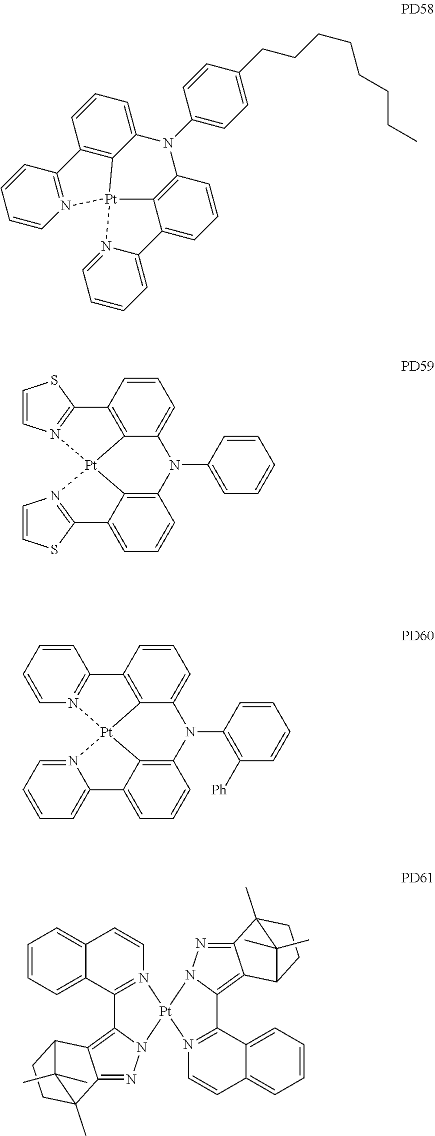

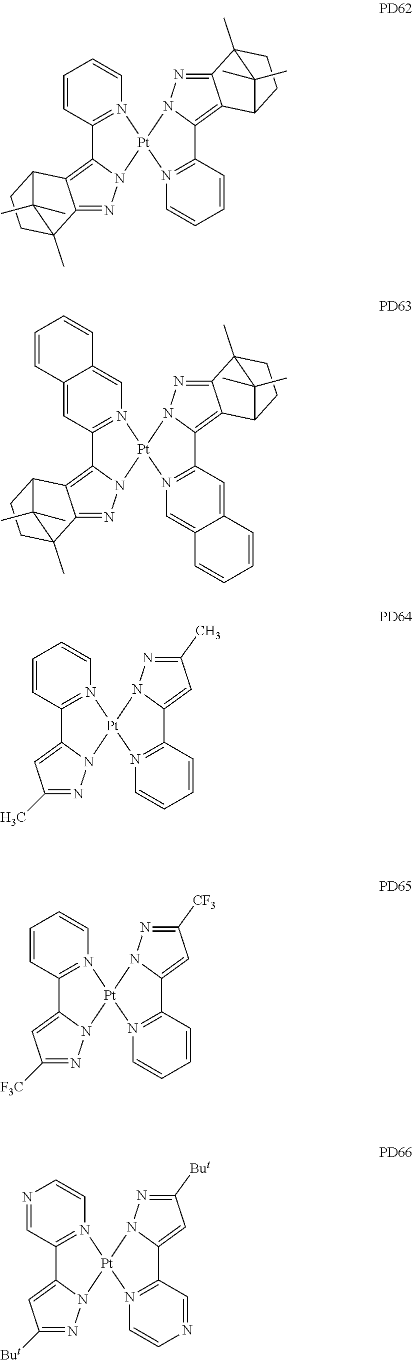

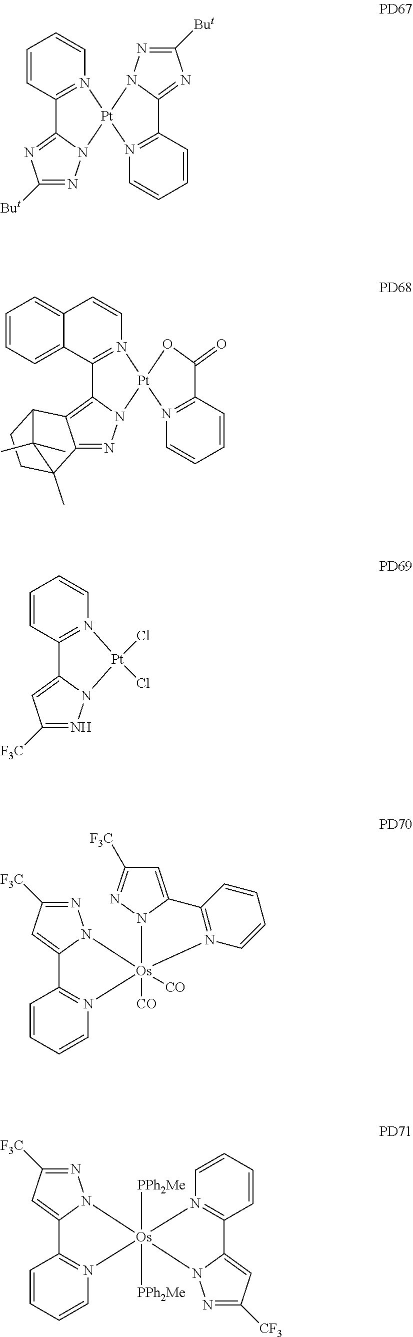

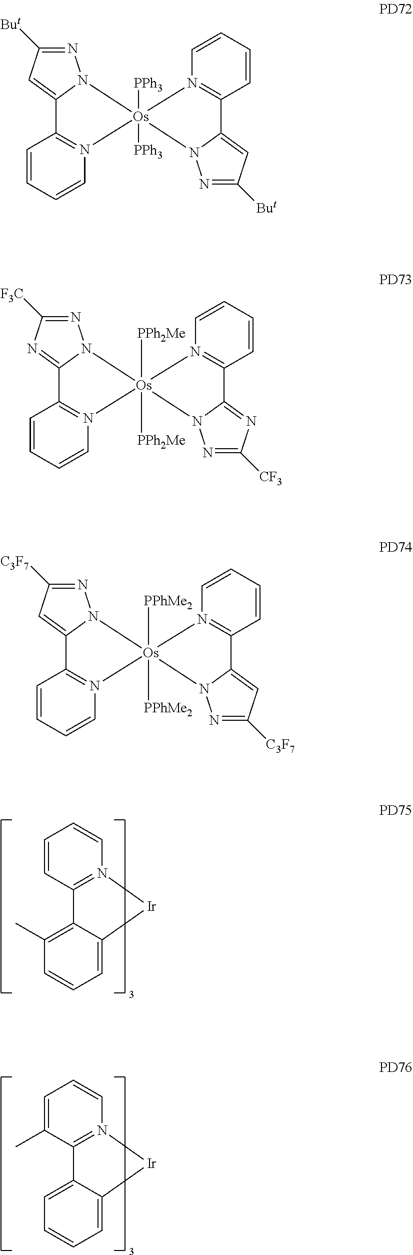

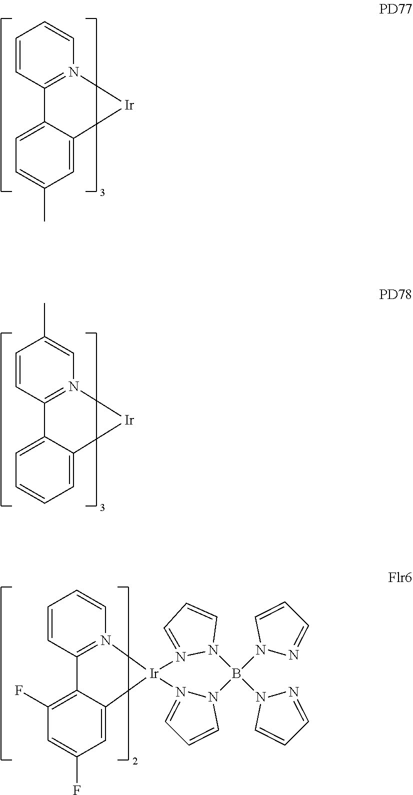

The expression “(an organic layer) includes at least one heterocyclic compound” as used herein may include a case in which “(an organic layer) includes identical heterocyclic compounds represented by Formula 1” and a case in which “(an organic layer) includes two or more different heterocyclic compounds represented by Formula 1”.

For example, the organic layer may include, as the heterocyclic compound, only Compound 1. In this regard, Compound 1 may exist only in the emission layer of the organic light-emitting device. In one or more embodiments, the organic layer may include, as the heterocyclic compound, Compound 1 and Compound 2. In this case, Compound 1 and Compound 2 may be present in an identical layer (for example, Compound 1 and Compound 2 may all be present in an emission layer), or different layers (for example, Compound 1 may be present in an emission layer and Compound 2 may be present in a hole blocking layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

The term “organic layer” used herein refers to a single layer and/or a plurality of layers between the first electrode and the second electrode of the organic light-emitting device. The “organic layer” may include, in addition to an organic compound, an organometallic complex including metal.

FIGURE is a schematic cross-sectional view of an organic light-emitting device 10 according to an embodiment. Hereinafter, the structure of an organic light-emitting device according to an embodiment and a method of manufacturing an organic light-emitting device according to an embodiment will be described in connection with FIGURE. The organic light-emitting device 10 includes a first electrode 11, an organic layer 15, and a second electrode 19, which are sequentially stacked.

A substrate may be additionally located under the first electrode 11 or above the second electrode 19. For use as the substrate, any substrate that is used in organic light-emitting devices available in the art may be used, and the substrate may be a glass substrate or a transparent plastic substrate, each having excellent mechanical strength, thermal stability, transparency, surface smoothness, ease of handling, and water resistance.

In one or more embodiments, the first electrode 11 may be formed by depositing or sputtering a material for forming the first electrode 11 on the substrate. The first electrode 11 may be an anode. The material for forming the first electrode 11 may be materials with a high work function to facilitate hole injection. The first electrode 11 may be a reflective electrode, a semi-transmissive electrode, or a transmissive electrode. The material for forming the first electrode 11 may be indium tin oxide (ITO), indium zinc oxide (IZO), tin oxide (SnO₂), or zinc oxide (ZnO). In one or more embodiments, the material for forming the first electrode 11 may be metal, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multi-layered structure including two or more layers. For example, the first electrode 11 may have a three-layered structure of ITO/Ag/ITO, but the structure of the first electrode 11 is not limited thereto.

The organic layer 15 is located on the first electrode 11.

The organic layer 15 may include a hole transport region, an emission layer, and an electron transport region.

The hole transport region may be between the first electrode 11 and the emission layer.

The hole transport region may include at least one a hole injection layer, a hole transport layer, an electron blocking layer, and a buffer layer.

The hole transport region may include only either a hole injection layer or a hole transport layer. In one or more embodiments, the hole transport region may have a hole injection layer/hole transport layer structure or a hole injection layer/hole transport layer/electron blocking layer structure, wherein, for each structure, each layer is sequentially stacked in this stated order from the first electrode 11.

When the hole transport region includes a hole injection layer (HIL), the hole injection layer may be formed on the first electrode 11 by using one or more suitable methods, for example, vacuum deposition, spin coating, casting, and/or Langmuir-Blodgett (LB) deposition.

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10⁻³ torr, and a deposition rate of about 0.01 Å/sec to about 100 Å/sec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 rpm to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include at least one m-MTDATA, TDATA, 2-TNATA, NPB, β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANT/PSS), a compound represented by Formula 201 below, a compound represented by Formula 202 below, or any combination thereof:

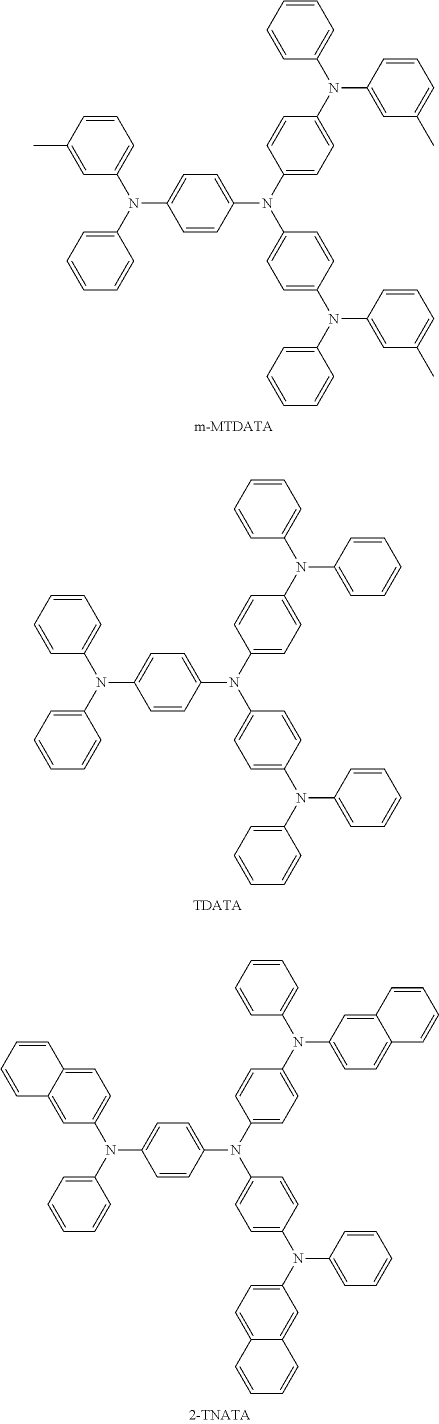

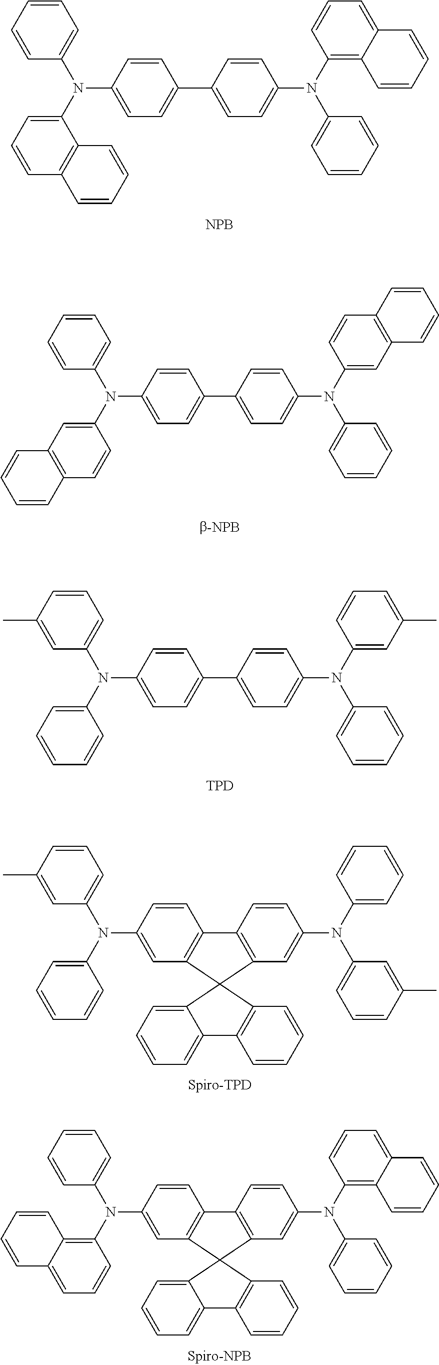

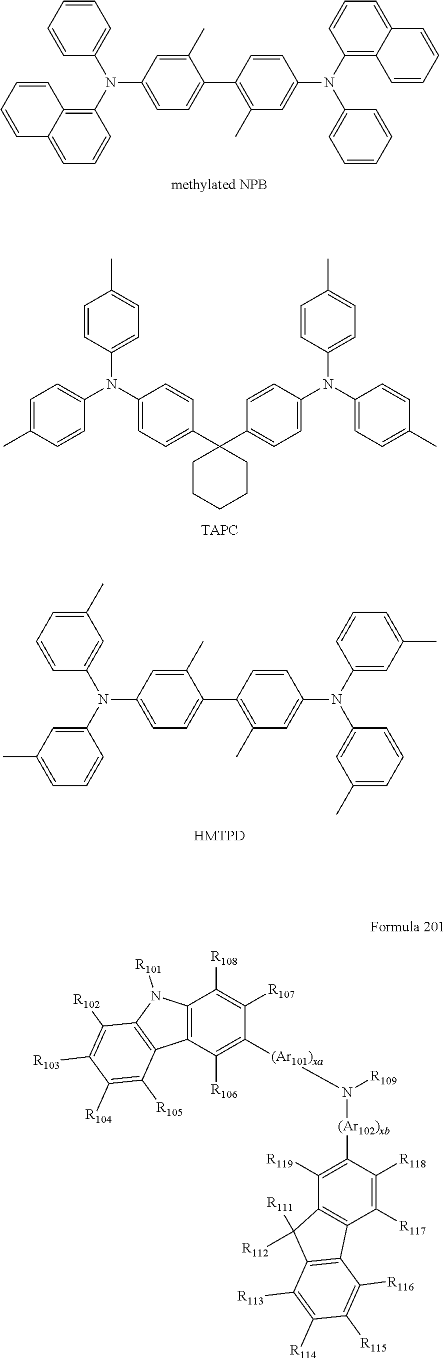

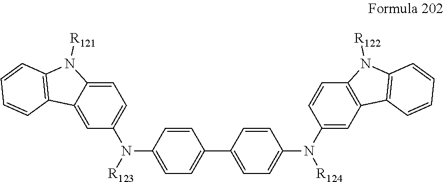

Ar₁₀₁ to Ar₁₀₂ in Formula 201 may each independently be:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, a pentacenylene group, or any combination thereof; or

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, a pentacenylene group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a heterocycloalkyl group, a C₂-C₁₀ heterocycloalkenyl group, a C₈-C₈₀ aryl group, a C₈-C₈₀ aryloxy group, a C₈-C₈₀ arylthio group, a C₁-C₆₀ heteroaryl group, a monovalent non-aromatic condensed polycyclic group, a monovalent non-aromatic condensed heteropolycyclic group, or any combination thereof.

xa and xb in Formula 201 may each independently be an integer from 0 to 5, or 0, 1, or 2. For example, xa may be 1 and xb may be 0, but xa and xb are not limited thereto.

R₁₀₁ to R₁₀₈, R₁₁₁ to R₁₁₉ and R₁₂₁ to R₁₂₄ in Formulae 201 and 202 may each independently be:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, etc.) a C₁-C₁₀ alkoxy group (for example, a methoxy group, an ethoxy group, a propoxy group, a butoxy group, a pentoxy group, etc.), or any combination thereof;

a C₁-C₁₀ alkyl group, a C₁-C₁₀ alkoxy group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, or any combination thereof;

a phenyl group, a naphthyl group, an anthracenyl group, a fluorenyl group, a pyrenyl group, or any combination thereof; or

a phenyl group, a naphthyl group, an anthracenyl group, a fluorenyl group, a pyrenyl group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, or any combination thereof, but embodiments of the present disclosure are not limited thereto.

R₁₀₉ in Formula 201 may be:

a phenyl group, a naphthyl group, an anthracenyl group, or a pyridinyl group; or a phenyl group, a naphthyl group, an anthracenyl group, or a pyridinyl group, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, a pyridinyl group, or any combination thereof.

According to an embodiment, the compound represented by Formula 201 may be represented by Formula 201A below, but embodiments of the present disclosure are not limited thereto:

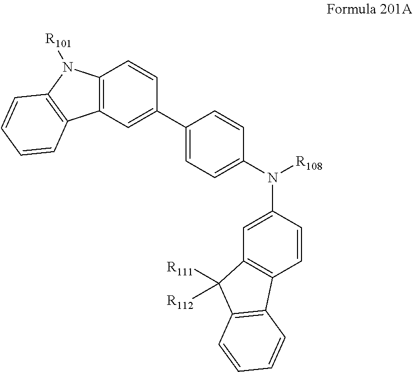

R₁₀₁, R₁₁₁, R₁₁₂, and R₁₀₉ in Formula 201A may be understood by referring to the description provided herein.

For example, the compound represented by Formula 201, and the compound represented by Formula 202 may include compounds HT1 to HT20 illustrated below, but are not limited thereto:

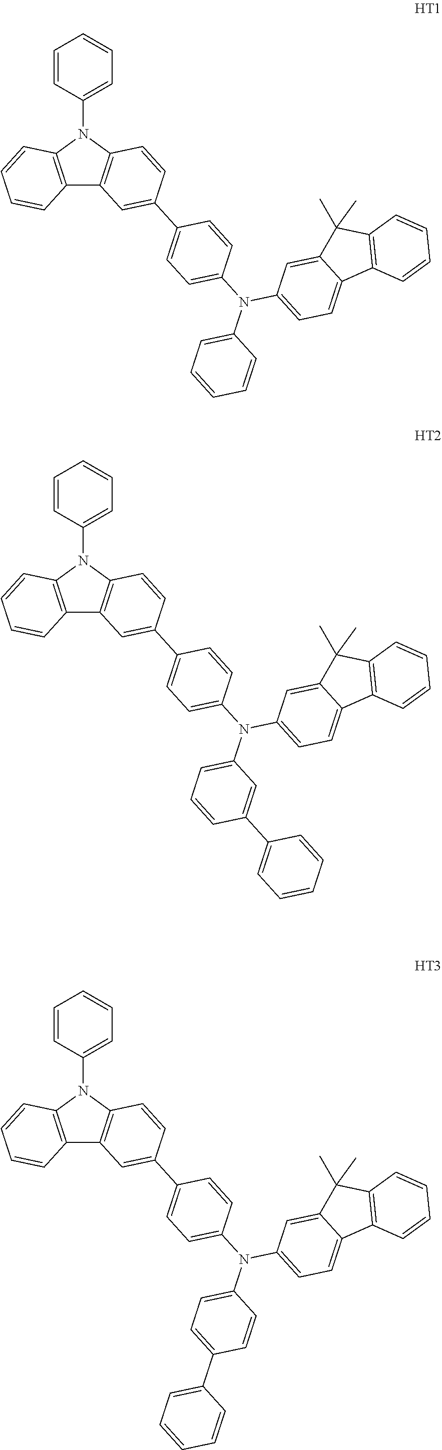 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

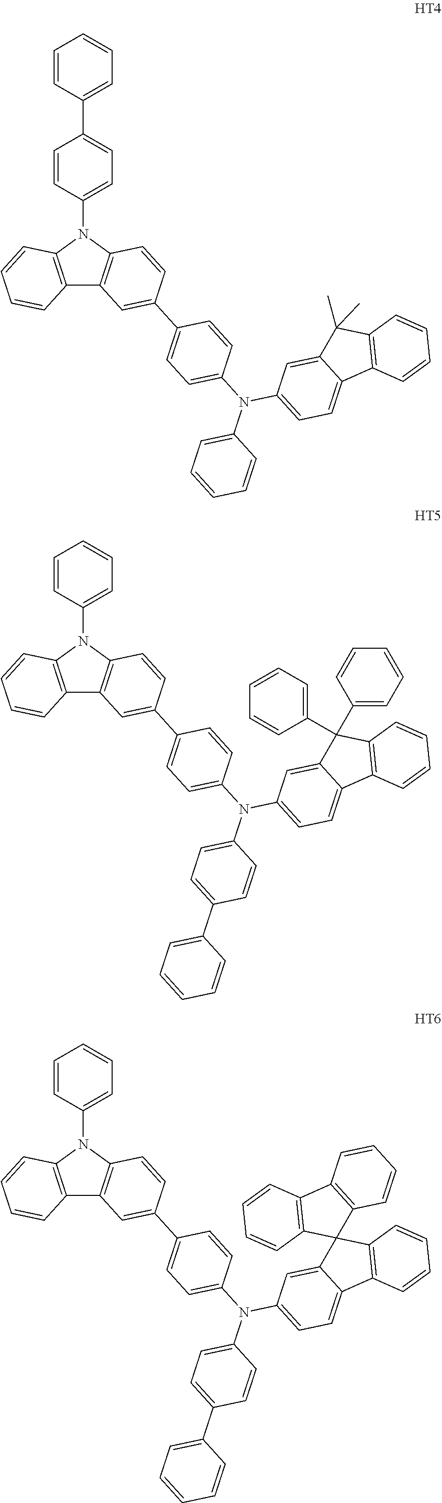 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

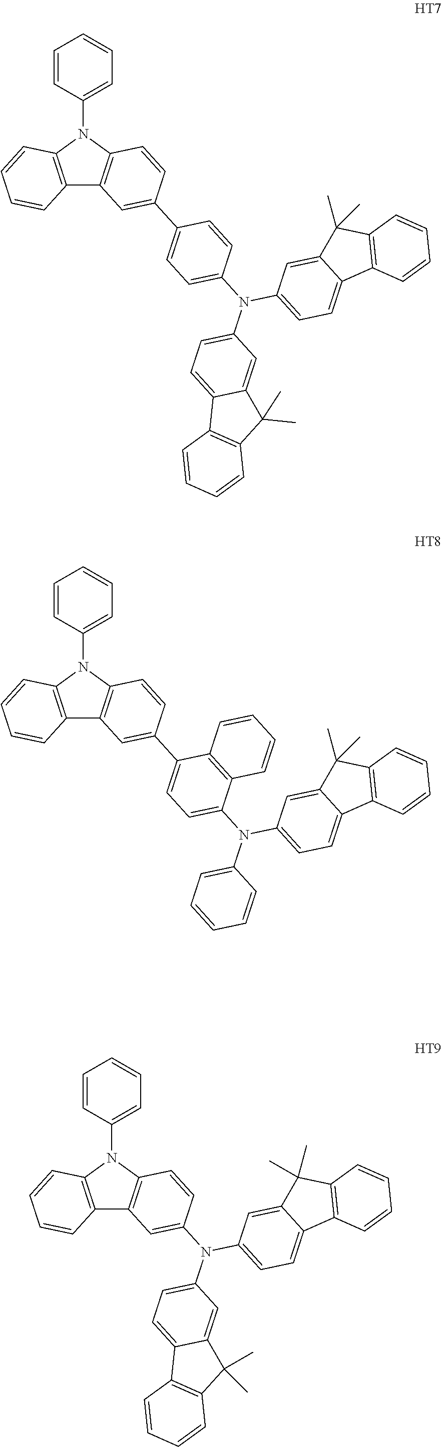 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

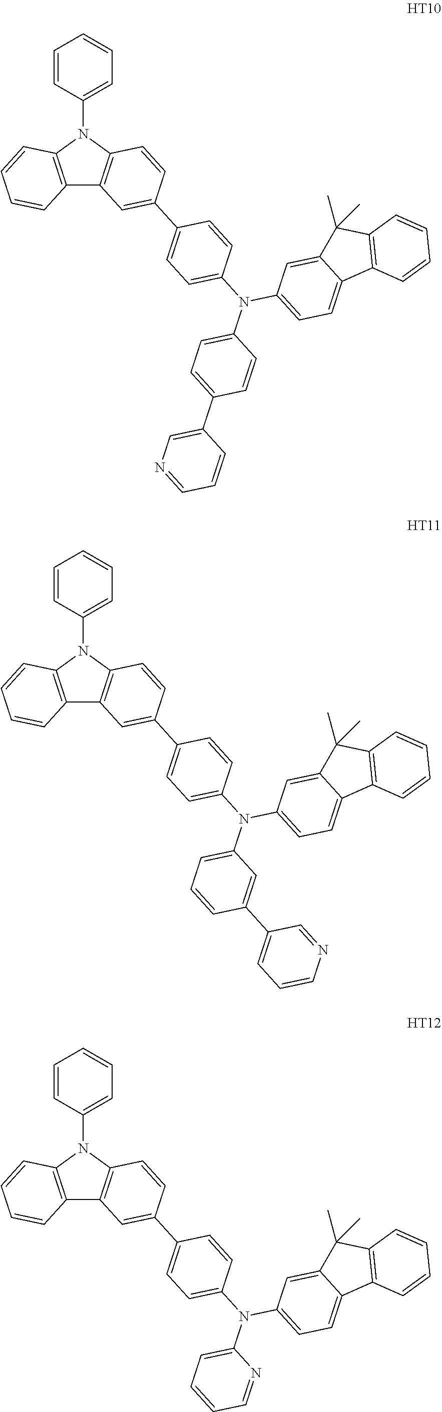 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

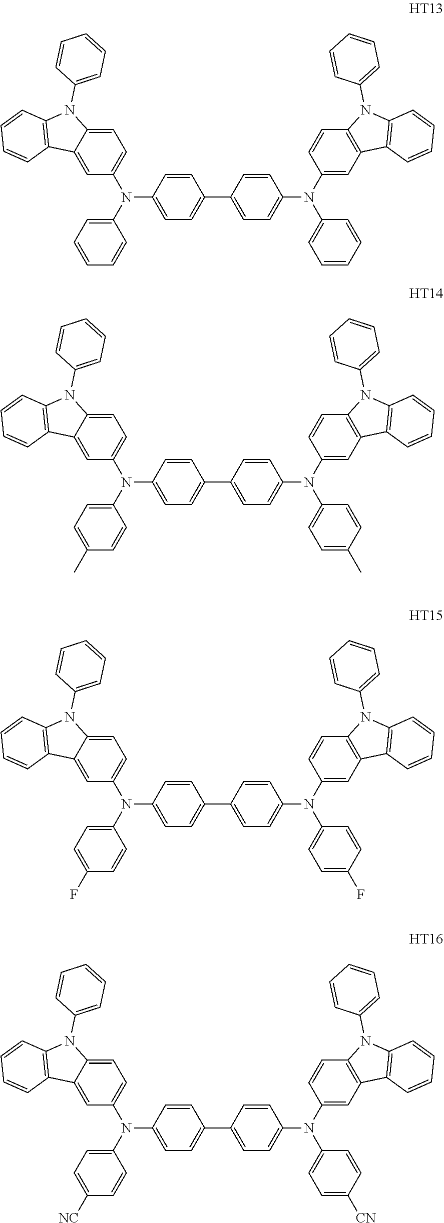 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

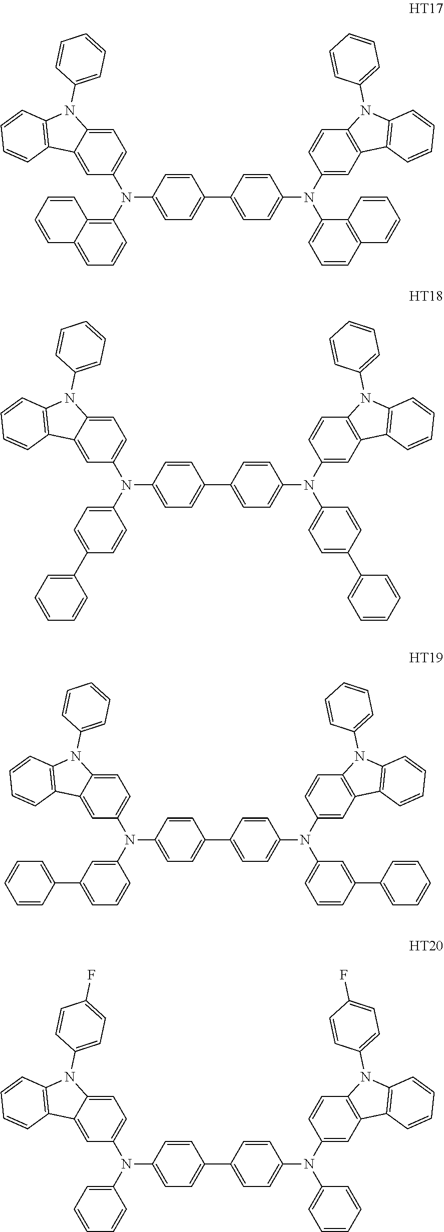 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes at least one of a hole injection layer and a hole transport layer, a thickness of the hole injection layer may be in a range of about 100 Å to about 10,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer and the hole transport layer are within these ranges, satisfactory hole transporting characteristics may be obtained without a substantial increase in driving voltage.

The hole transport region may further include, in addition to these materials, a charge-generation material for improvement of conductive properties. The charge-generation material may be homogeneously or non-homogeneously dispersed in the hole transport region.

The charge-generation material may be, for example, a p-dopant. The p-dopant may be one of a quinone derivative, a metal oxide, and a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide; and a cyano group-containing compound, such as Compound HT-D1 or Compound HT-D2 below, but are not limited thereto.

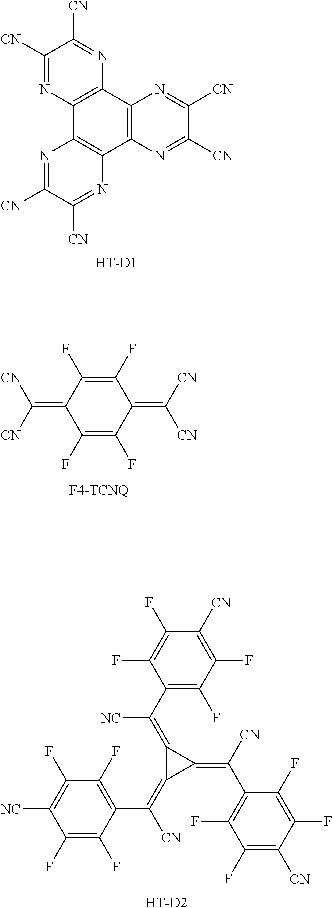

The hole transport region may include a buffer layer.

Also, the buffer layer may compensate for an optical resonance distance according to a wavelength of light emitted from the emission layer, and thus, efficiency of a formed organic light-emitting device may be improved.

The hole transport region may further include an electron blocking layer. The electron blocking layer may include a material available in the art, for example, mCP, but embodiments of the present disclosure are not limited.

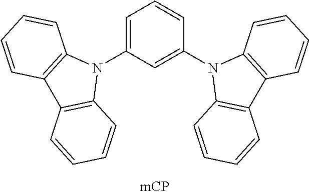 [SMILES: c1cc(-n2c3ccccc3c3ccccc32)cc(-n2c3ccccc3c3ccccc32)c1]

The thickness of the electron blocking layer may be about 50 Å to about 1,000 Å, for example about 70 Å to about 500 Å. When the thickness of the electron blocking layer is within the range described above, the electron blocking layer may have satisfactory electron blocking characteristics without a substantial increase in driving voltage.

Then, an emission layer (EML) may be formed on the hole transport region by vacuum deposition, spin coating, casting, LB deposition, or the like. When the emission layer is formed by vacuum deposition or spin coating, the deposition or coating conditions may be similar to those applied in forming the hole injection layer although the deposition or coating conditions may vary according to a material that is used to form the emmission layer.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The emission layer may include the heterocyclic compound represented by Formula 1.

For example, the emission layer may include the heterocyclic compound represented by Formula 1 alone.

In one or more embodiments, the emission layer may include

the heterocyclic compound represented by Formula 1, and may further include: i) the second compound (for example, a compound different from the heterocyclic compound represented by Formula 1); ii) the organometallic compound represented by Formula 81; or iii) any combination thereof.

The heterocyclic compound represented by Formula 1, the second compound, and the organometallic compound represented by Formula 81 may each be the same as described above.

When the emission layer includes a host and a dopant, the amount of the dopant may be in the range of about 0.01 to about 20 parts by weight based on 100 parts by weight of the emission layer. However, the amount of the dopant included in the emission layer is not limited thereto. When the amount of the dopant satisfies the range, it may be possible to realize emission without extinction phenomenon.

When the emission layer includes the heterocyclic compound represented by Formula 1 and the second compound, the weight ratio of the heterocyclic compound represented by formula 1 to the second compound may be in the range of about 1:99 to about 99:1, for example, about 70:30 to about 30:70. In one or more embodiments, the weight ratio of the heterocyclic compound represented by Formula 1 and the second compound may be in the range of about 60:40 to about 40:60. When the weight ratio of the heterocyclic compound represented by formula 1 to the second compound in the emission layer is within this range, the charge transport balance in the emission layer may be effectively performed.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

Then, an electron transport region may be located on the emission layer.

The electron transport region may include at least one a hole blocking layer, an electron transport layer and an electron injection layer.

For example, the electron transport region may have a hole blocking layer/electron transport layer/electron injection layer structure or an electron transport layer/electron injection layer structure, and the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP, Bphen, or any combination thereof, but embodiments of the present disclosure are not limited thereto.

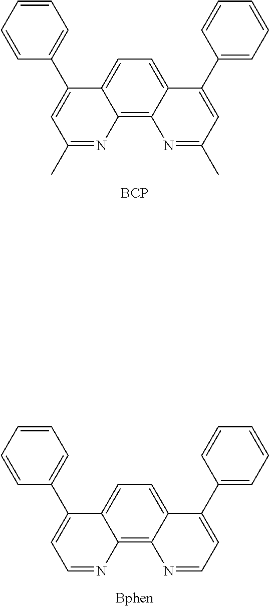

In one or more embodiments, the hole blocking layer may include the heterocyclic compound represented by Formula 1.

A thickness of the hole blocking layer may be in a range of about 20 Å to about 1,000 Å, for example, about 30 Å to about 300 Å. When the thickness of the hole blocking layer is within these ranges, the hole blocking layer may have excellent hole blocking characteristics without a substantial increase in driving voltage.

The electron transport layer may further include at least one BCP, Bphen, Alq₃, BAlq, TAZ, NTAZ, or any combination thereof.

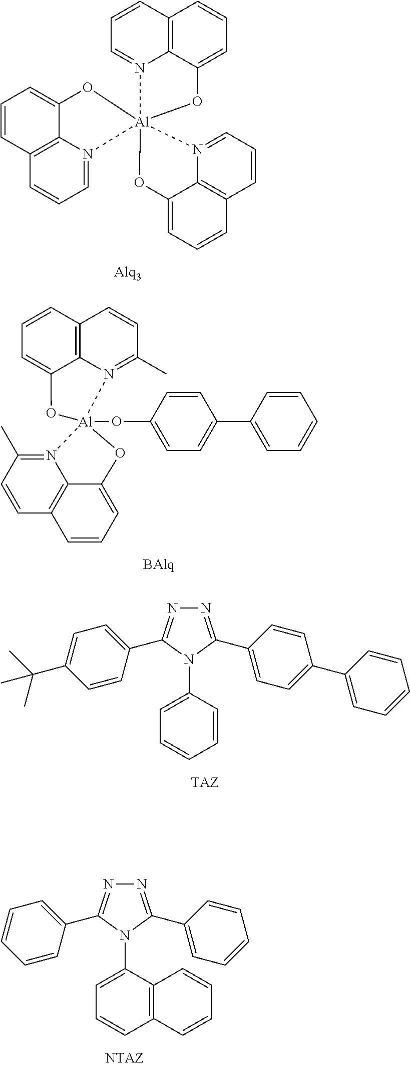

in one or more embodiments, the electron transport layer may include at least one of ET1, ET2, ET3, or any combination thereof, but are not limited thereto:

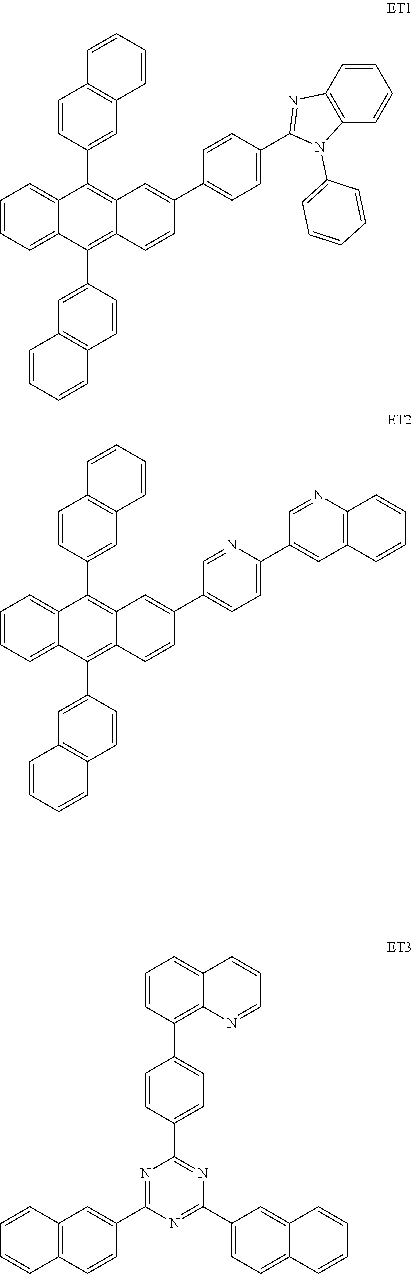 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

A thickness of the electron transport layer may be in a range of about 100 Å to about 1,000 Å, for example, about 150 Å to about 500 Å. When the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a L₁ complex. The L₁ complex may include, for example, Compound ET-D1 (lithium 8-hydroxyquinolate, LiQ) or ET-D2:

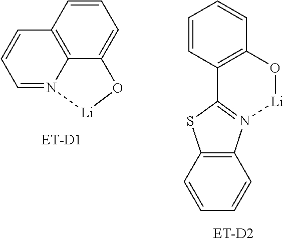

The electron transport region may include an electron injection layer (EIL) that promotes the flow of electrons from the second electrode 19 thereinto.

The electron injection layer may include at least one LiQ, LiF, NaCl, CsF, Li₂O, BaO, or any combination thereof.

A thickness of the electron injection layer may be in a range of about 1 Å to about 100 Å, and, for example, about 3 Å to about 90 Å. When the thickness of the electron injection layer is within the range described above, the electron injection layer may have satisfactory electron injection characteristics without a substantial increase in driving voltage.

The second electrode 19 is located on the organic layer 15. The second electrode 19 may be a cathode. A material for forming the second electrode 19 may be metal, an alloy, an electrically conductive compound, or a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be used as the material for forming the second electrode 19. To manufacture a top-emission type light-emitting device, a transmissive electrode formed using ITO or IZO may be used as the second electrode 19.

Hereinbefore, the organic light-emitting device has been described with reference to FIGURE, but embodiments of the present disclosure are not limited thereto.

According to one embodiment, provided is an electronic apparatus including a substrate, and an organic light-emitting device located on the substrate. The organic light-emitting device is the same as described above.

According to one embodiment, the electronic apparatus may further include a color conversion layer,

The color conversion layer may be located on at least one propagation direction of light emitted from the organic light-emitting device, and may include a quantum dot.

The quantum dot is a particle having a crystal structure of several to several tens of nanometers, and includes hundreds to thousands of atoms.

Since the quantum dot is very small in size, a quantum confinement effect may occur. The quantum confinement effect refers to a phenomenon in which a band gap of an object becomes large when the object becomes smaller than a nanometer size. Accordingly, when light having a wavelength having an energy intensity that is greater than the band gap of the quantum dot is irradiated to the quantum dot, the quantum dot is excited by absorbing the light and emits light having a specific wavelength and transits to the ground state. In this case, the wavelength of the emitted light has a value corresponding to the band gap.

The quantum dot may be a semiconductor material. For example, the quantum dot may include a Group II-VI semiconductor compound, a Group III-V semiconductor compound, a Group IV-VI semiconductor compound, a Group IV element or compound, or a combination thereof. The Group II-VI semiconductor compound may be, for example, a binary compound which may be CdS, CdSe, CdTe, ZnS, ZnSe, ZnTe, ZnO, HgS, HgSe, HgTe, or a combination thereof; a ternary compound which may be CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, or a combination thereof; or a quaternary compound which may be CdHgZnTe, CdZnSeS, CdZnSeTe, CdZnSTe, CdHgSeS, CdHgSeTe, CdHgSTe, HgZnSeS, HgZnSeTe, HgZnSTe, or a combination thereof. The Group III-V semiconductor compound may be, for example, a binary compound which may be GaN, GaP, GaAs, GaSb, AlN, AlP, AlAs, AlSb, InN, InP, InAs, InSb, or a combination thereof; a ternary compound which may be GaNP, GaNAs, GaNSb, GaPAs, GaPSb, AlNP, AlNAs, AlNSb, AlPAs, AlPSb, InNP, InNAs, InNSb, InPAs, InPSb, GaAlNP, or a combination thereof; or a quaternary compound which may be GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, or a combination thereof. The Group IV-VI semiconductor compound may be, for example, a binary compound which may be SnS, SnSe, SnTe, PbS, PbSe, PbTe, or a combination thereof; a ternary compound which may be SnSeS, SnSeTe, SnSTe, PbSeS, PbSeTe, PbSTe, SnPbS, SnPbSe, SnPbTe, or a combination thereof; or a quaternary compound which may be SnPbSSe, SnPbSeTe, SnPbSTe, or a combination thereof. The Group IV element or compound may be, for example Si, Ge, SiC, SiGe, or a combination thereof.

The quantum dot may have a core structure or a core-shell structure, or a core-shell-shell structure. The quantum dot core may have a diameter of about 1 nm to several tens nm depending on a composition material. The quantum dot core-shell structure may be, for example, a CdSe/CdS structure or an InP/ZnS structure. The quantum dot core-shell-shell structure may be, for example, a CdSe/CdS/ZnS structure.

The quantum dot may adjust the color of emitted light according to the particle size. Therefore, the quantum dot may emit various emission colors such as blue, red, or green.

In addition, the form of the quantum dot is not particularly limited. For example, the quantum dot may be a spherical, cubic, pyramid, or multi-arm nanoparticle. In one or more embodiments, the quantum dot may have the form of nanotubes, nanowires, nanofibers, nanoplate particles, or the like.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” used herein refers to a monovalent group represented by-OA₁₀₁ (wherein A₁₀₁ is the C₁-C₆₀ alkyl group), and examples thereof include a methoxy group, an ethoxy group, and an isopropyloxy group.

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one N, O, P, Si, B, Se, Ge, Te, S, or any combination thereof as a ring-forming atom and 1 to 10 carbon atoms, and non-limiting examples thereof include a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one N, O, P, Si, B, Se, Ge, Te, S, or any combination thereof as a ring-forming atom, 2 to 10 carbon atoms, and at least one (e.g., carbon-carbon) double bond in its ring. Examples of the C₂-C₁₀ heterocycloalkenyl group are a 2,3-dihydrofuranyl group, and a 2,3-dihydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other. The C₇-C₆₀ alkylaryl group refers to a C₆-C₆₀ aryl group substituted with at least one C₁-C₆₀ alkyl group.

The term “C₁-C₆₀ heteroaryl group” as used herein refers to a monovalent group having a heterocyclic aromatic system that has at least one heteroatom N, O, P, Si, B, Se, Ge, Te, S, or any combination thereof as a ring-forming atom, and 1 to 60 carbon atoms. The term “C₁-C₆₀ heteroarylene group” as used herein refers to a divalent group having a heterocyclic aromatic system that has at least one heteroatom N, O, P, B, Se, Ge, Te, S, or any combination thereof as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₆-C₆₀ heteroaryl group and the C₆-C₆₀ heteroarylene group each include two or more rings, the rings may be fused to each other. The C₂-C₆₀ alkylheteroaryl group refers to a C₁-C₅₉ heteroaryl group substituted with at least one C₁-C₅₉ alkyl group.

The term “C₆-C₆₀ aryloxy group” as used herein indicates-OA₁₀₂ (wherein A₁₀₂ is the C₆-C₆₀ aryl group), and the term “C₆-C₆₀ arylthio group” as used herein indicates-SA₁₀₃ (wherein A₁₀₃ is the C₆-C₆₀ aryl group).

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 2 to 60 carbon atoms) having two or more rings condensed to each other, at least one heteroatom N, O, P, Si, B, Se, Ge, Te, S, or any combination thereof other than carbon atoms, as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₃₀ carbocyclic group” as used herein refers to a saturated or unsaturated cyclic group having, as a ring-forming atom, 5 to 30 carbon atoms only. The C₅-C₃₀ carbocyclic group may be a monocyclic group or a polycyclic group.

The term “C₁-C₃₀ heterocyclic group” as used herein refers to a saturated or unsaturated cyclic group having, as a ring-forming atom, at least one heteroatom N, O, Si, P, B, Se, Ge, Te, S, or any combination thereof other than 1 to 30 carbon atoms. The C₁-C₃₀ heterocyclic group may be a monocyclic group or a polycyclic group.

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₇-C₆₀ alkylaryl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted monovalent non-aromatic condensed polycyclic group, and the substituted monovalent non-aromatic condensed heteropolycyclic group is:

deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or any combination thereof;

a C₁-C₆₀ alkyl group, a C₂-C₆₀ alkenyl group, a C₂-C₆₀ alkynyl group, a C₁-C₆₀ alkoxy group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkyl heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or any combination thereof;

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkyl heteroaryl group, a monovalent non-aromatic condensed polycyclic group, a monovalent non-aromatic condensed heteropolycyclic group, or any combination thereof;

a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkyl heteroaryl group, a monovalent non-aromatic condensed polycyclic group, a monovalent non-aromatic condensed heteropolycyclic group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkyl heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or any combination thereof; or

—N(Q₃₁)(Q₃₂), —Si(Q₃₃)(Q₃₄)(Q₃₅), —B(Q₃₆)(Q₃₇), —P(═O)(Q₃₈)(Q₃₉), or any combination thereof,

wherein Q₁ to Q₉, Q₁₁ to Q₁₉, Q₂₁ to Q₂₉, and Q₃₁ to Q₃₉ may each independently be hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₁-C₆₀ alkyl group substituted with at least one deuterium, a C₁-C₆₀ alkyl group, a C₆-C₆₀ aryl group, or any combination thereof, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryl group substituted with at least one deuterium, a C₁-C₆₀ alkyl group, a C₆-C₆₀ aryl group, or any combination thereof, a C₆-C₆₀ aryloxy group, a C₆-C₆₀ arylthio group, a C₁-C₆₀ heteroaryl group, a C₂-C₆₀ alkyl heteroaryl group, a monovalent non-aromatic condensed polycyclic group, a monovalent non-aromatic condensed heteropolycyclic group, or any combination thereof.

Hereinafter, a compound and an organic light-emitting device according to embodiments are described in detail with reference to Synthesis Example and Examples. However, the organic light-emitting device is not limited thereto. The wording “‘B’ was used instead of ‘A’” used in describing Synthesis Examples means that an amount of ‘A’ used was identical to an amount of ‘B’ used, in terms of a molar equivalent.

### EXAMPLES

### Synthesis Example 1: Synthesis of Compound 1

Compound 1 was synthesized according to the following reaction scheme.

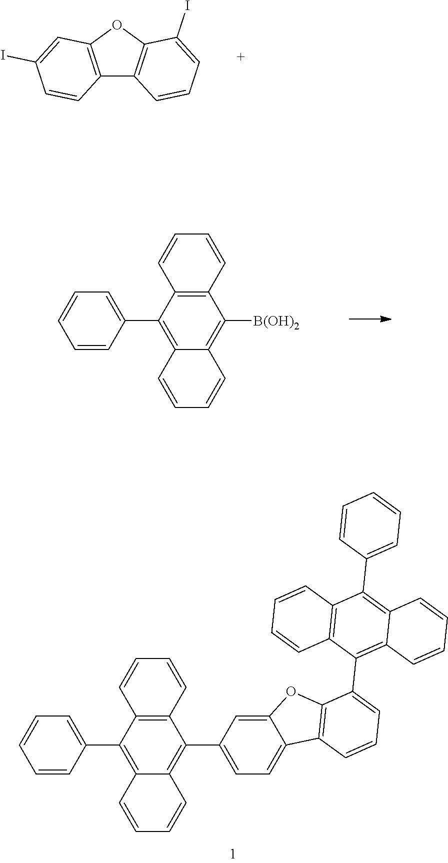 [SMILES: Ic1ccc2c(c1)oc1c(I)cccc12 ; OB(O)c1c2ccccc2c(-c2ccccc2)c2ccccc12 ; c1ccc(-c2c3ccccc3c(-c3ccc4c(c3)oc3c(-c5c6ccccc6c(-c6ccccc6)c6ccccc56)cccc34)c3ccccc23)cc1]

3,6-diiododibenzofuran (5 g, 11.9 mmol), (10-phenylanthracen-9-yl)boronic acid (7.8 g, 26.2 mmol), palladium tetrakistriphenylphosphine (Pd(PPh₃)₄, 2.8 g, 2.4 mmol), and potassium phosphate tribasic (K₃PO₄, 15.2 g, 71.4 mmol) were added to 50 ml of toluene, 12 ml of ethanol, and 12 ml of distilled water, and the mixture was refluxed. After the reaction was completed, the reaction mixture was cooled to room temperature, the organic layer was extracted with toluene, dried using anhydrous sodium sulfate (Na₂SO₄), and filtered using a silica filter. The filtrate was concentrated and recrystallize with toluene and ethyl acetate to obtain Compound 1. (3.8 g, 5.6 mmol, yield 47%)

LCMS (calculated: 672.25, found (M+1): 673.245 m/z) Synthesis Example 2: Synthesis of Compound 113

Compound 113 was synthesized using the same method as in Synthesis Example 1, except that when Compound 1 was synthesized, 6-bromo-2-iododibenzofuran was used instead of 3,6-diiododibenzofuran. (Yield of 45%)

LCMS (calculated: 672.25, found(M+1): 673.245 m/z)

### Synthesis Example 3: Synthesis of Compound 134

Compound 134 was synthesized in the same manner as used in Synthesis Example 1, except that when Compound 1 was synthesized, 6-bromo-2-iododibenzofuran was used instead of 3,6-diiododibenzofuran, and 2-(10-(4,4,5,5-tetramethyl-1,3,2-dioxaborolan-2-ypanthracen-9-yl)benzonitrile was used instead of (10-phenylanthracene-9-yl)boronic acid, (Yield of 35%)

LCMS (calculated: 722.24, found(M+1): 723.241 m/z)

### Synthesis Example 4: Synthesis of Compound 171

Compound 171 was synthesized according to the following reaction scheme.

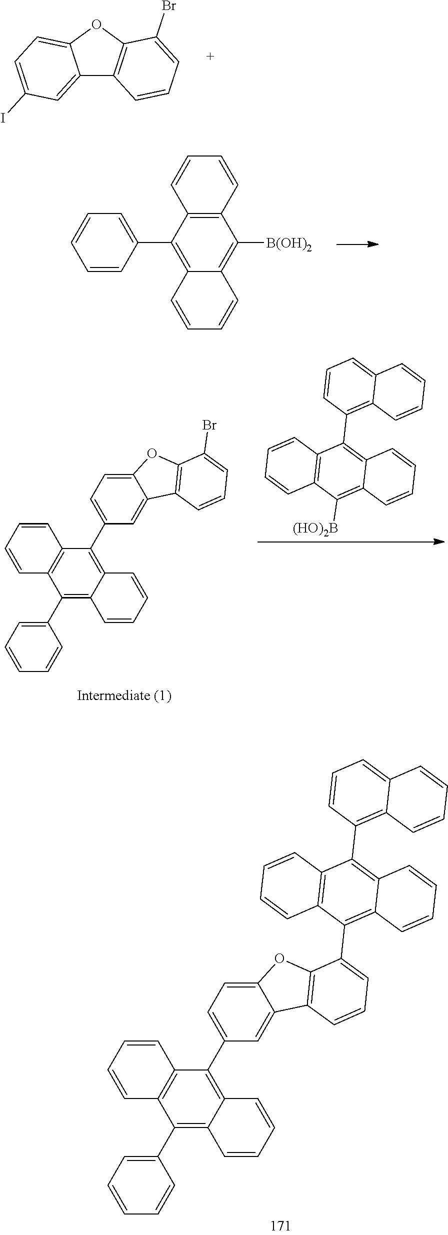

### (1) Synthesis of Intermediate (1)

6-bromo-2-iododibenzofuran (10 g, 26.8 mmol), (10-phenylanthracen-9-yl)boronic acid (8.8 g, 29.5 mmol), palladium tetrakistriphenylphosphine (Pd(PPh₃)₄, 6.2 g, 5.4 mmol), and potassium carbonate (K₂CO₃, 11.1 g, 80.4 mmol) were added to 120 ml of tetrahydrofuran (THF) and 40 ml of distilled water, and the mixture was refluxed. After the reaction was completed, the reaction mixture was cooled to room temperature, the organic layer was extracted with toluene, dried using anhydrous sodium sulfate (Na₂SO₄) and concentrated, and subjected to silica column chromatography, thereby obtaining Intermediate 1. (7.3 g, 14.6 mmol, yield 55%)

### (2) Synthesis of Compound 171

Compound 171 was synthesized using the same method as in Synthesis Example 1, except that when Compound 1 was synthesized, Intermediate 1 was used instead of 3,6-diiododibenzofuran, and 1.2 eq of 10-(1-naphthyl)anthracene-9-boronic acid was used instead of (10-phenylanthracen-9-yl)boronic acid. (Yield of 52%)

LCMS (calculated: 722.26, found(M+1): 723.257 m/z)

### Synthesis Example 5: Synthesis of Compound 1121

Compound 1121 was synthesized using the same method as in Synthesis Example 1, except that when Compound 1 was synthesized, 6-bromo-2-iododibenzoselenophene was used instead of 3,6-diiododibenzofuran. (Yield of 38%)

LCMS (calculated: 736.17, found (M+1): 737.155 m/z)

### Synthesis Example 6: Synthesis of Compound 2102

Compound 2102 was synthesized using the same method as in Synthesis Example 1, except that when Compound 1 was synthesized, 6-bromo-2-iododibenzofuran was used instead of 3,6-diiododibenzofuran and (10-butylanthracene-9-yl)boronic acid was used instead of (10-phenylanthracen-9-yl)boronic acid. (Yield of 46%)

LCMS (calculated: 632.31, found(M+1): 633.307 m/z)

### Synthesis Example 7: Synthesis of Compound A

Compound A was synthesized using the same method as in Synthesis Example 1, except that when Compound 1 was synthesized, 2,8-dibromodibenzofuran was used instead of 3,6-diiododibenzofuran. (Yield of 43%)

LCMS (calculated: 672.25, found (M+1): 673.245 m/z)

### Synthesis Example 8: Synthesis of Compound B

Compound B was synthesized using the same method as in Synthesis Example 1, except that when Compound 1 was synthesized, 6-bromo-2-iododibenzothiophene was used instead of 3,6-diiododibenzofuran. (Yield of 41%)

LCMS (calculated: 688.22, found (M+1): 689.167 m/z)

### Example 1

A patterned ITO glass substrate (50 mm×50 mm×0.7 mm) was ultrasonically cleaned in acetone, isopropyl alcohol, and distilled water, each for 20 minutes, and then, heat-treated at a temperature of 250° C. for 10 minutes.

Then, HATCN was deposited on the ITO electrode (anode) on the glass substrate at a deposition rate of 1 Å/sec to form a hole injection layer having a thickness of 100 Å, and NPB was deposited on the hole injection layer at a deposition rate of 1 Å/sec to form a hole transport layer having a thickness of 800 Å.

Then, mCP was deposited on the hole transport layer at a deposition rate of 1 Å/sec to form an electron blocking layer having a thickness of 50 Å.

Compound 1 (host) and Compound D1 (dopant) were co-deposited on the electron blocking layer respectively at deposition rates of 0.97 Å/sec and 0.3 Å/sec to form an emission layer having a thickness of 200 Å.

DPEPO and LiQ (at the ratio of 1:1) were co-deposited on the emission layer at the deposition rate of 0.5 Å/sec to form an electron transport layer having a thickness of 300 Å, and then LiQ was deposited on the electron transport layer at the deposition rate of 0.5 Å/sec to form an electron injection layer having a thickness of 10 Å, and then, Al was vacuum-deposited on the electron injection layer to form a second electrode(cathode) having a thickness of 1000 Å, thereby completing the manufacture of an organic light-emitting device having the structure of ITO/HATCN (100 Å)/NPB (800 Å)/mCP (50 Å)/Compound 1+Compound D1 (3%) (200 Å)/DPEPO:LiQ (300 Å)/LiQ (10 Å)/Al (1000 Å)

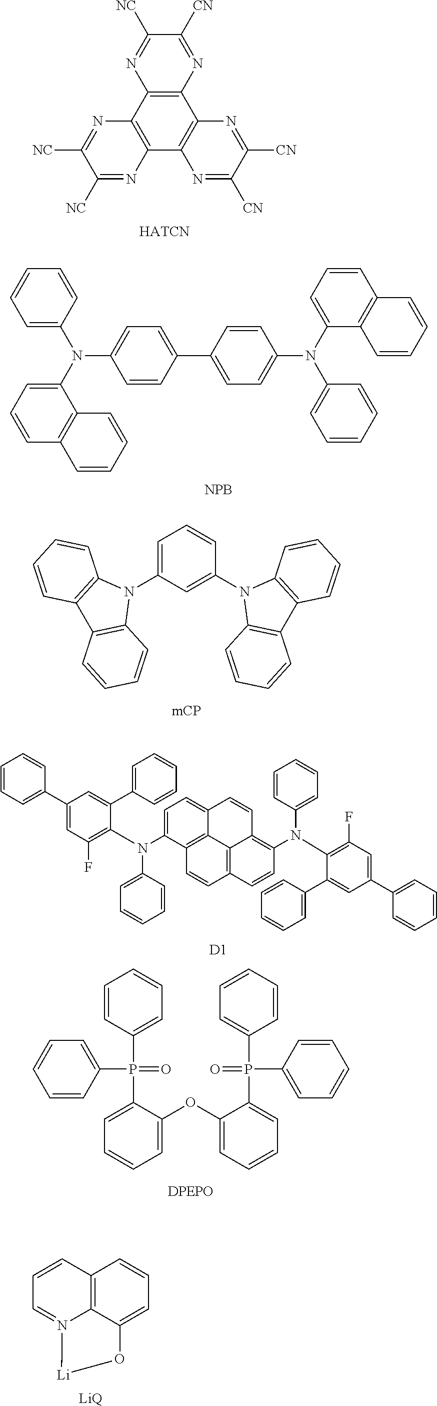

### Examples 2 to 5 and Comparative Examples 1 to 2

Organic light-emitting devices were manufactured in the same manner as in Example 1, except that Compounds shown in Table 2 were each used instead of Compound 1 as a host in forming an emission layer.

### Evaluation Example 1: Characterization of Organic Light-Emitting Device

For each of the organic light-emitting devices manufactured according to Examples 1 to 5 and Comparative Examples 1 to 2, the photoluminescence quantum efficiency (PLQY), external quantum efficiency (EQE), TTF ratio and lifespan (T₉₅) were evaluated as relative values. The results are shown in Table 2. This evaluation was performed using a current-voltage meter (Keithley 2400) and a luminescence meter (Minolta Cs-1,000A), and the lifespan (T₉₅)(at 6000 nit) was evaluated as a relative value by measuring, as a relative value, the amount of time that elapsed until luminance was reduced to 95% of the initial brightness of 100%. The TTF ratio was obtained by: obtaining a graph of 1/square root of the TrEL (1/sqrt (TrEL)) with respect to time by measuring the decay of the transient electroluminescence (TrEL) and then taking the square of the inverse of the y-intercept value of the 1/sqrt (TrEL) from 500 ns to 4000 ns in the graph.

TABLE 2

| | Emission | EQE | lifespan(T₉₅) | TTF |
|---|---|---|---|---|
| | layer | (relative | (relative | ratio |
| No. | host | value) | value) | (%) |
| Example 1 | Compound 1 | 100 | 100 | 23.8 |
| Example 2 | Compound 113 | 107.6 | 106.3 | 29.8 |
| Example 3 | Compound 171 | 107.9 | 116.3 | 28.5 |
| Example 4 | Compound 1121 | 113.4 | 53.2 | 26.3 |
| Example 5 | Compound 2102 | 117.3 | 60.0 | 24.2 |
| Comparative | Compound A | 102.5 | 28.8 | 22 |
| Example 1 | | | | |
| Comparative | Compound B | 111.8 | 52.8 | 22.8 |
| Example 2 | | | | |

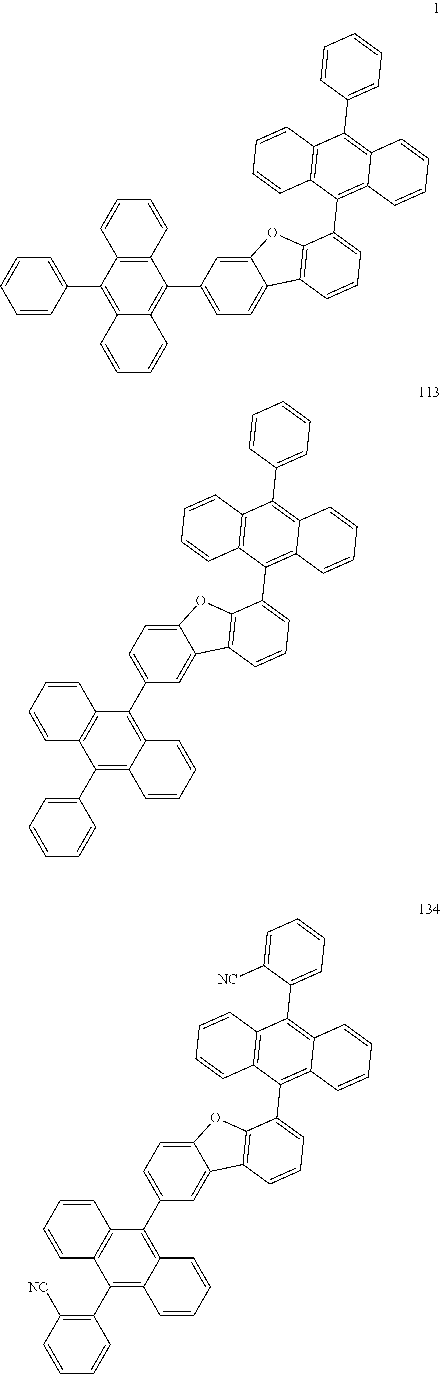 [SMILES: c1ccc(-c2c3ccccc3c(-c3ccc4c(c3)oc3c(-c5c6ccccc6c(-c6ccccc6)c6ccccc56)cccc34)c3ccccc23)cc1 ; c1ccc(-c2c3ccccc3c(-c3ccc4oc5c(-c6c7ccccc7c(-c7ccccc7)c7ccccc67)cccc5c4c3)c3ccccc23)cc1 ; N#Cc1ccccc1-c1c2ccccc2c(-c2ccc3oc4c(-c5c6ccccc6c(-c6ccccc6C#N)c6ccccc56)cccc4c3c2)c2ccccc12]

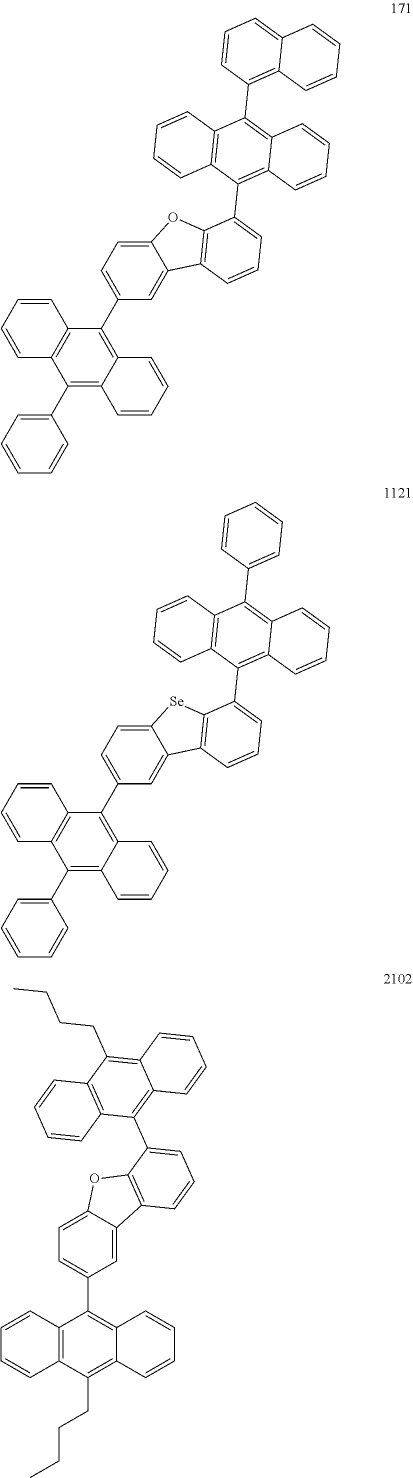

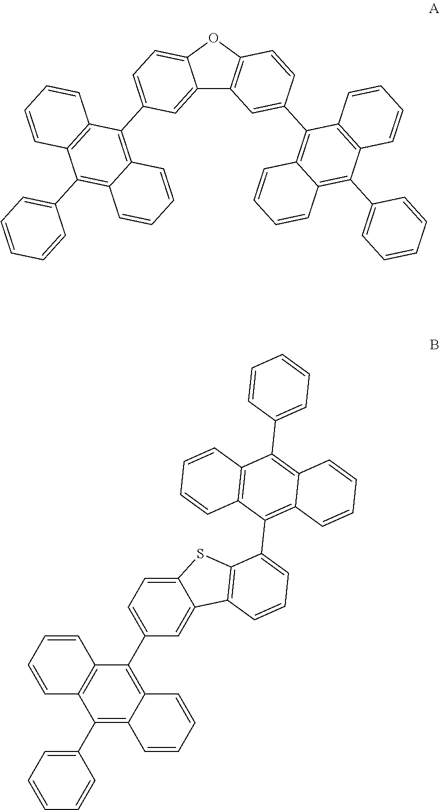 [SMILES: c1ccc(-c2c3ccccc3c(-c3ccc4oc5ccc(-c6c7ccccc7c(-c7ccccc7)c7ccccc67)cc5c4c3)c3ccccc23)cc1 ; c1ccc(-c2c3ccccc3c(-c3ccc4sc5c(-c6c7ccccc7c(-c7ccccc7)c7ccccc67)cccc5c4c3)c3ccccc23)cc1]

Table 2 shows that the organic light-emitting devices of Examples 1 to 5 have excellent external quantum efficiency and lifespan characteristics and high TTF ratios. In addition, the organic light-emitting devices of Example 1 to 5 showed better lifespan characteristics and higher TTF ratios than the organic light-emitting devices of Comparative Examples 1 and 2.

As described above, the heterocyclic compounds according to embodiments of the present disclosure have excellent electrical characteristics and thermal stability. Accordingly, an organic light-emitting device using the heterocyclic compound may have high efficiency, long lifespans, and high TTF ratios.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A heterocyclic compound represented by Formula 1 and having an asymmetric structure: 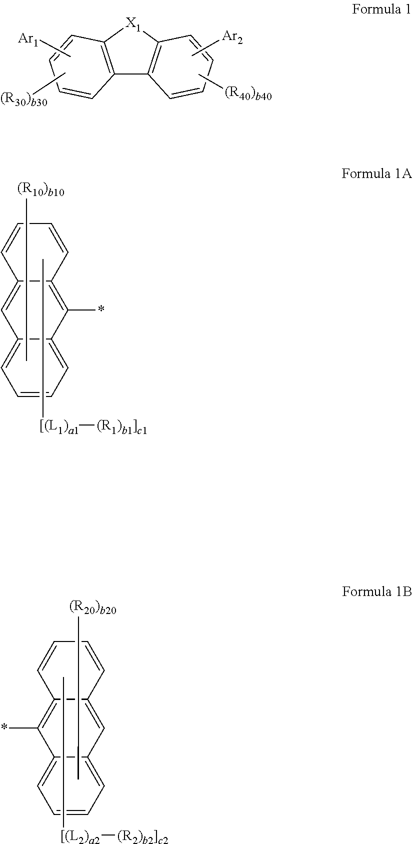
wherein, in Formulae 1, 1A, and 1B,
X₁ is O or Se,
Ar₁ is a group represented by Formula 1A, and Are is a group represented by Formula 1B,
L₁ and L₂ are each independently a substituted or unsubstituted C₅-C₃₀ carbocyclic group, a substituted or unsubstituted C₁-C₃₀ heterocyclic group, or any combination thereof,
a1 and a2 are each independently an integer from 0 to 3,
wherein when a1 is 2 or more, two or more of L₁(s) are identical to or different from each other, and when a2 is 2 or more, two or more of L₂(s) are identical to or different from each other,
R₁, R₂, R₁₀, R₂₀, R₃₀, and R₄₀ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), —P(═O)(Q₈)(Q₉), or any combination thereof,
b1 and b2 are each independently an integer from 1 to 5,
wherein when b1 is 2 or more, two or more of R₁(s) are identical to or different from each other, and when b2 is 2 or more, two or more of R₂ are identical to or different from each other,
b10 and b20 are each independently an integer from 1 to 8,
wherein, when b10 is 2 or more, two or more of R₁₀(s) are identical to or different from each other, and when b20 is 2 or more, two or more of R₂₀(s) are identical to or different from each other,
b30 and b40 are each independently an integer from 1 to 3,
wherein, when b30 is 2 or more, two or more of R₃₀(s) are identical to or different from each other, and when b40 is 2 or more, two or more of R₄₀(s) are identical to or different from each other,
c1 and c2 are each independently an integer from 1 to 8,
wherein, when c1 is 2 or more, two or more of-(L₁)ₐ₁-(R₁)_{b1} groups are identical to or different from each other, and when c2 is 2 or more, two or more of-(L₂)ₐ₂-(R₂)_{b2} groups are identical to or different from each other,
the sum of b10 and c1 is 9 and the sum of b20 and c2 is 9, and
at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, or any combination thereof;
a C₁-C₆₀ alkyl group, a C₂-C₆₀ alkenyl group, a C₂-C₆₀ alkynyl group, a C₁-C₆₀ alkoxy group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇), or any combination thereof;
a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or any combination thereof;
a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or any combination thereof, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), —B(Q₂₆)(Q₂₇), or any combination thereof; or
—Si(Q₃₁)(Q₃₂)(Q₃₃), —N(Q₃₄)(Q₃₅), —B(Q₃₆)(Q₃₇), or any combination thereof,
wherein Q₁ to Q₇, Q₁₁ to Q₁₇, Q₂₁ to Q₂₇, and Q₃₁ to Q₃₇ are each independently hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, or any combination thereof.

2. The heterocyclic compound of claim 1, wherein Ar₁ is represented by one of Formulae 1A-1 to 1A-5: 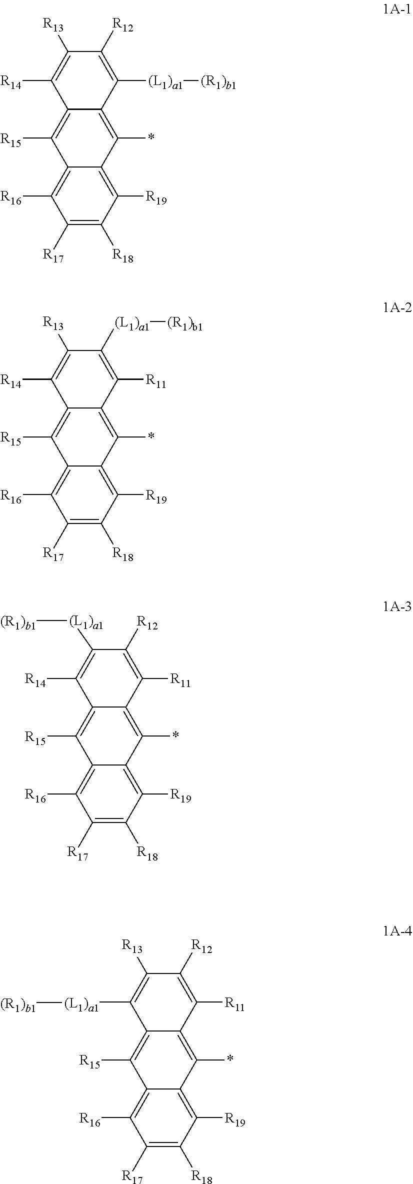 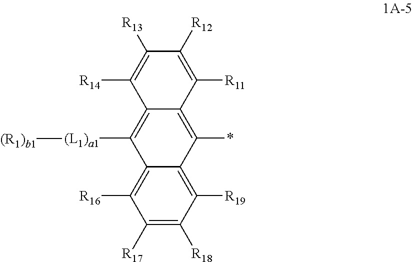
wherein, in Formulae 1A-1 to 1A-5,
L₁, a1, R₁, and b1 are the same as described in connection with claim 1,
R₁₁ to R₁₉ are the same as described in connection with R₁₀ in claim 1, and
* indicates a binding site to a neighboring atom.

3. The heterocyclic compound of claim 1, wherein Are is represented by one of Formulae 2A-1 to 2A-5: 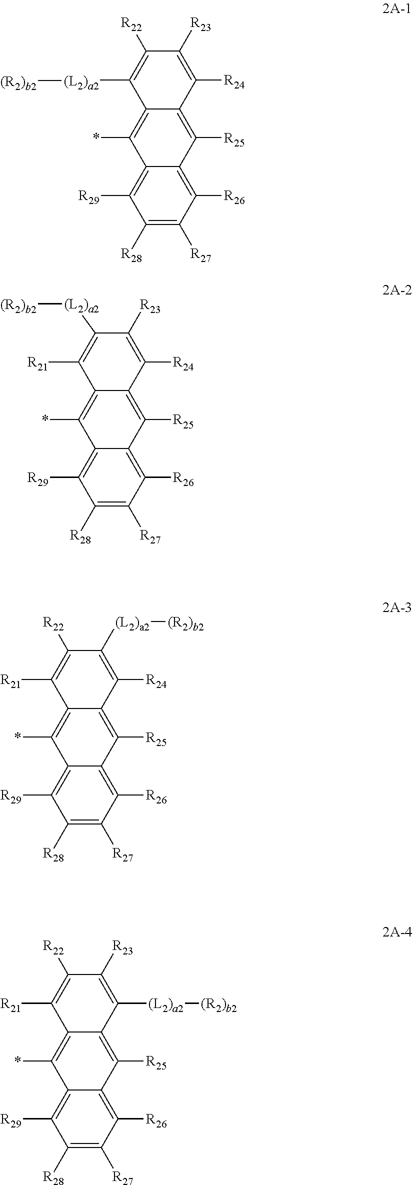 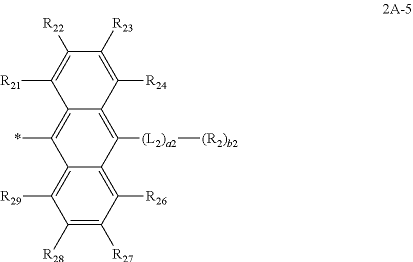
wherein, in Formulae 2A-1 to 2A-5,
L₂, a2, R₂, and b2 are the same as described in connection with claim 1,
R₂₁ to R₂₉ are the same as described in connection with R₂₀ in claim 1, and
* indicates a binding site to a neighboring atom.

4. The heterocyclic compound of claim 1, wherein the heterocyclic compound represented by Formula 1 is represented by one of Formulae 1-1 to 1-6: 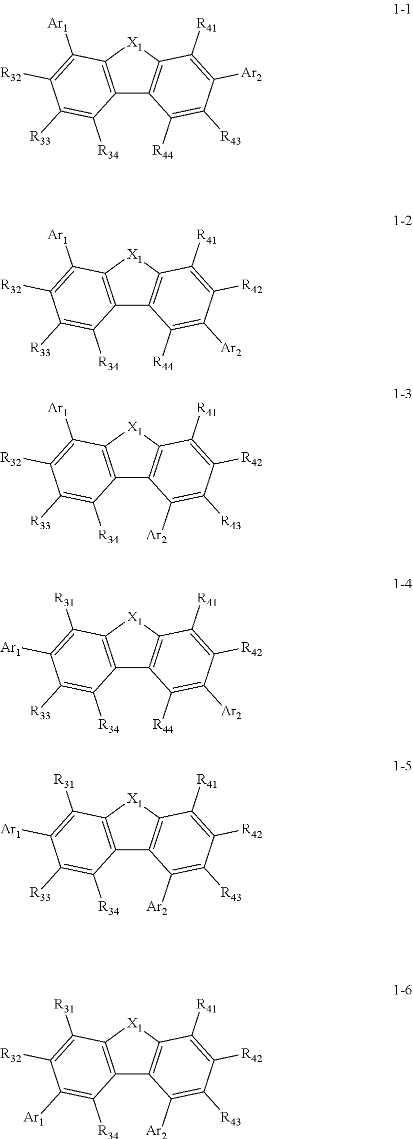
wherein, in Formulae 1-1 to 1-6,
X₁, Ar₁, and Ar₂ are the same as described in connection with claim 1,
R₃₁ to R₃₄ are the same as described in connection with R₃₀ in claim 1, and
R₄₁ to R₄₄ are the same as described in connection with R₄₀ in claim 1.

5. The heterocyclic compound of claim 1, wherein L₁ and L₂ are each independently:
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, a pentacenylene group, or any combination thereof; or
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, a pentacenylene group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or any combination thereof.

6. The heterocyclic compound of claim 1, wherein R₁ and R₂ are each independently deuterium, —F, a cyano group, a group represented by Formulae 9-1 to 9-19, a group represented by Formulae 10-1 to 10-208, or any combination thereof: 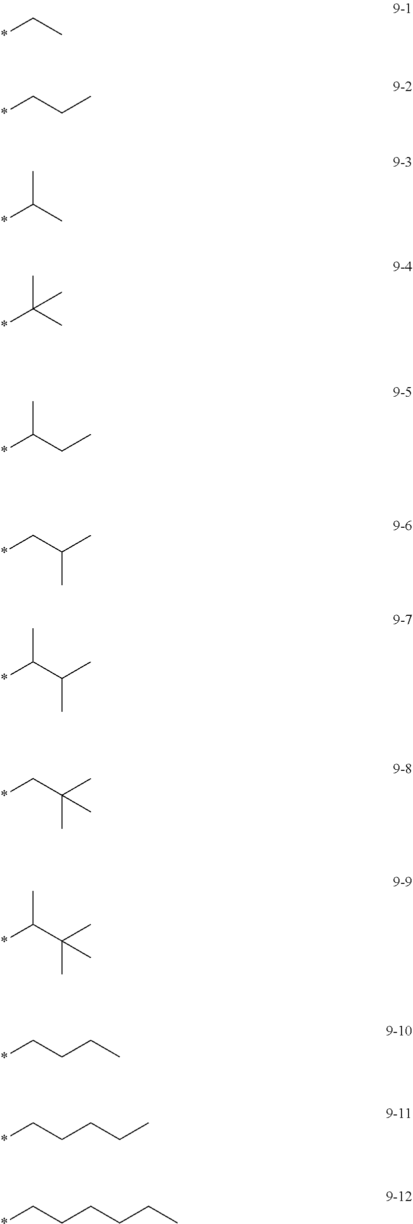 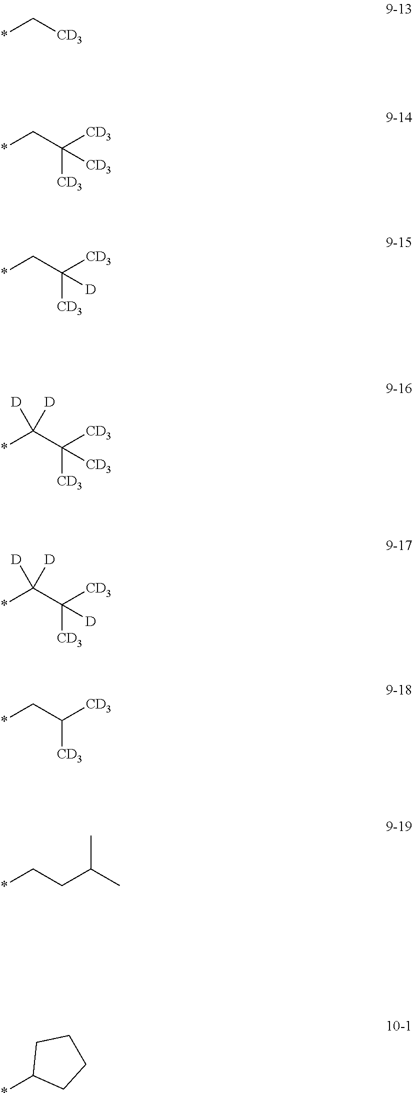 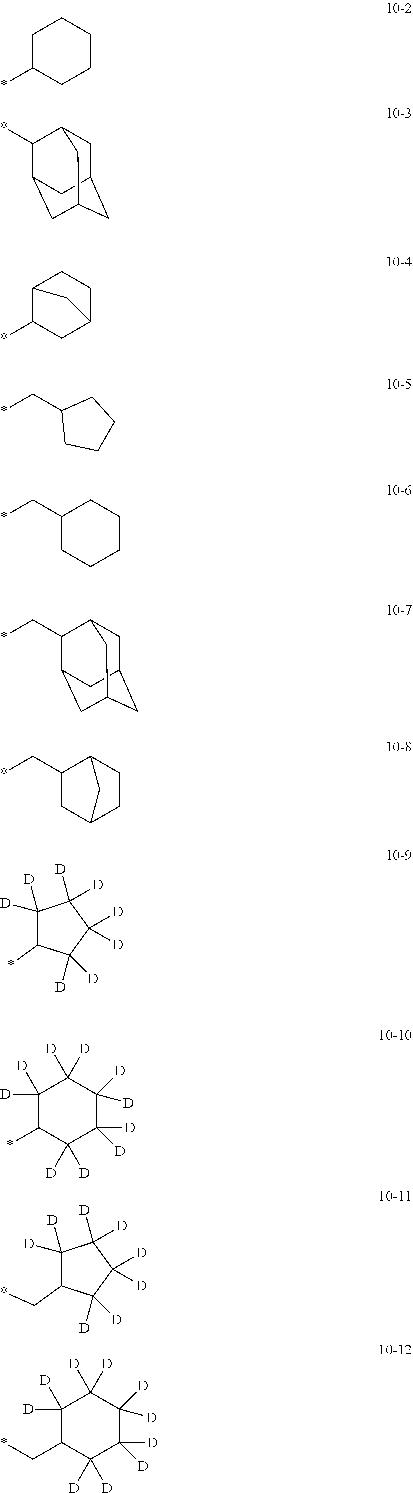 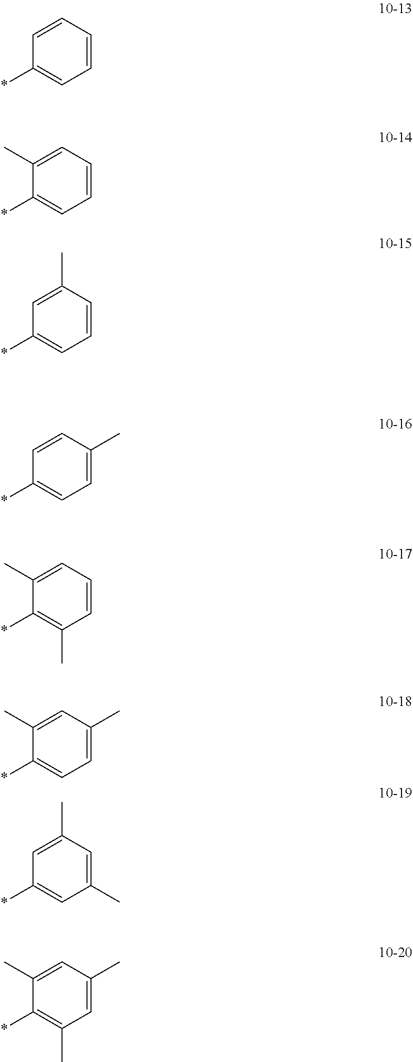 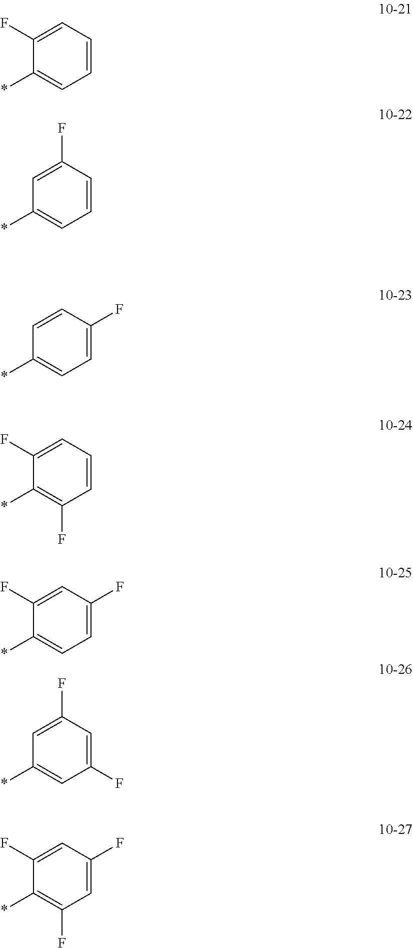 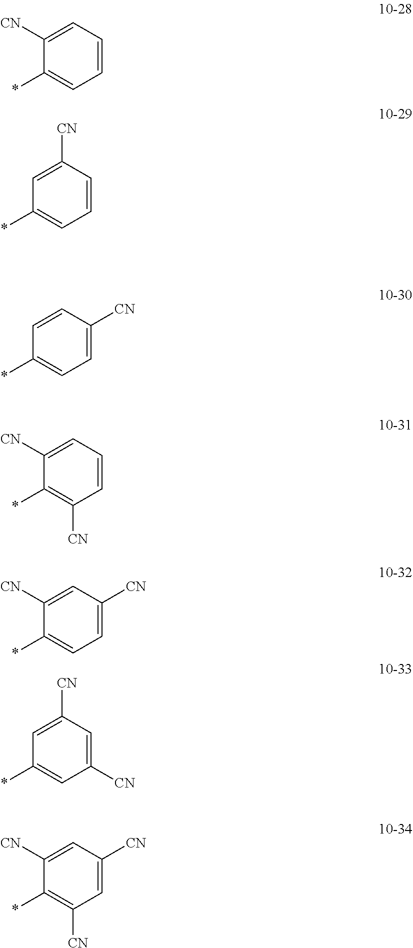 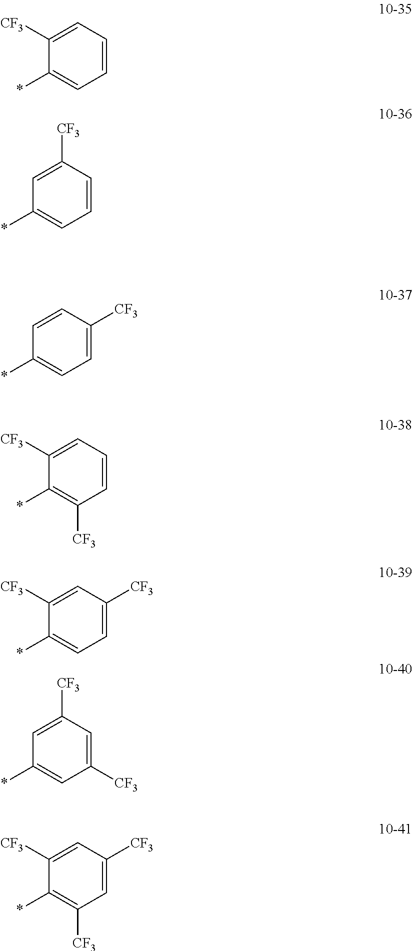 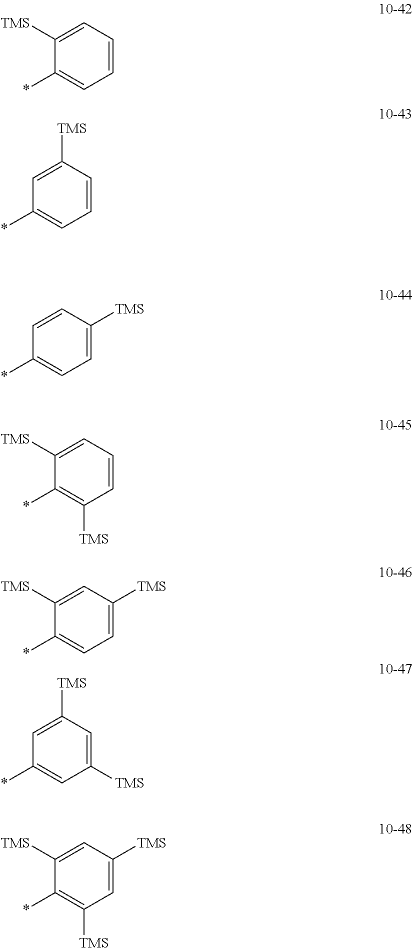 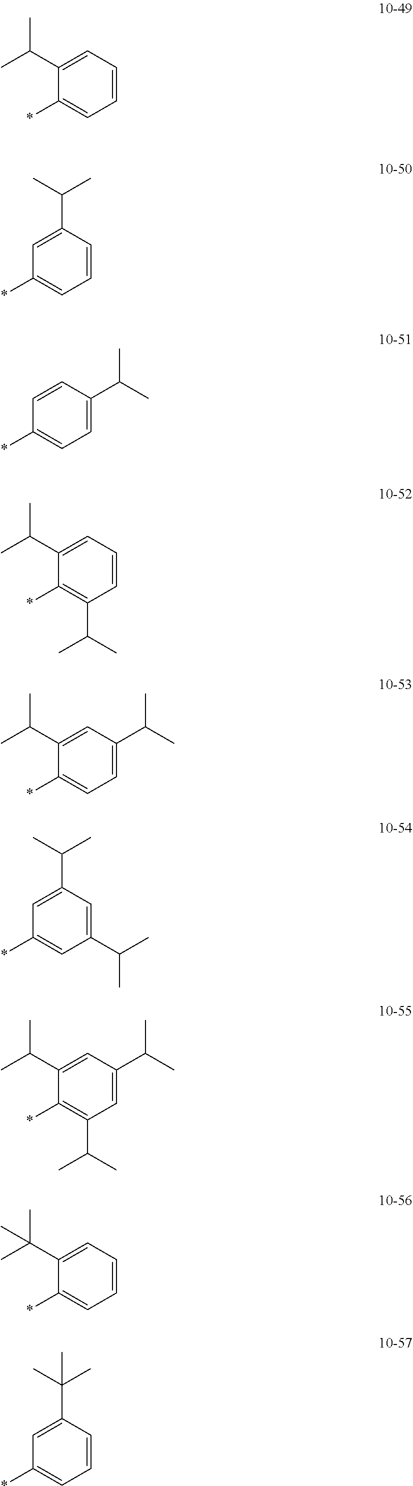 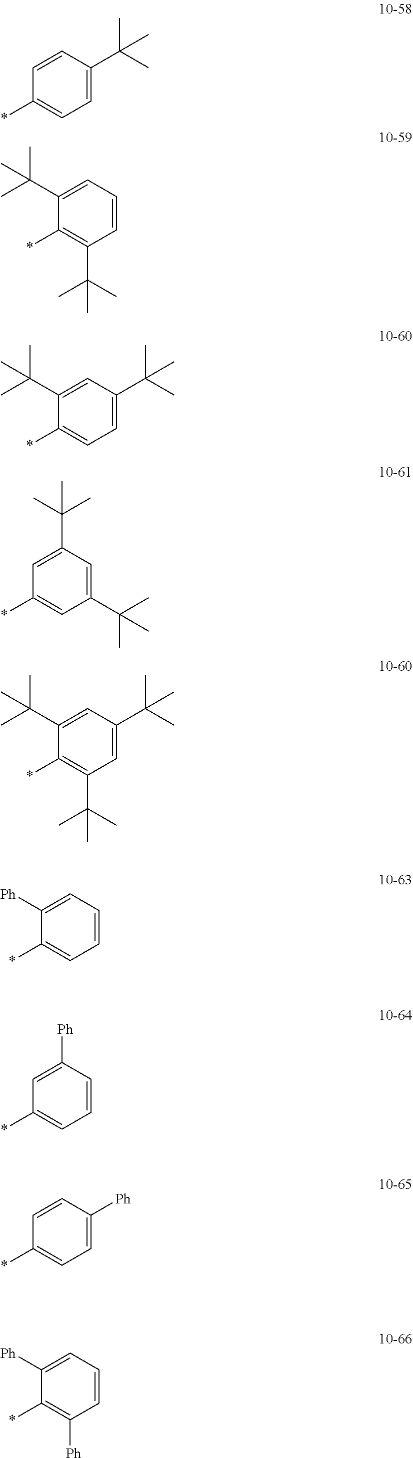 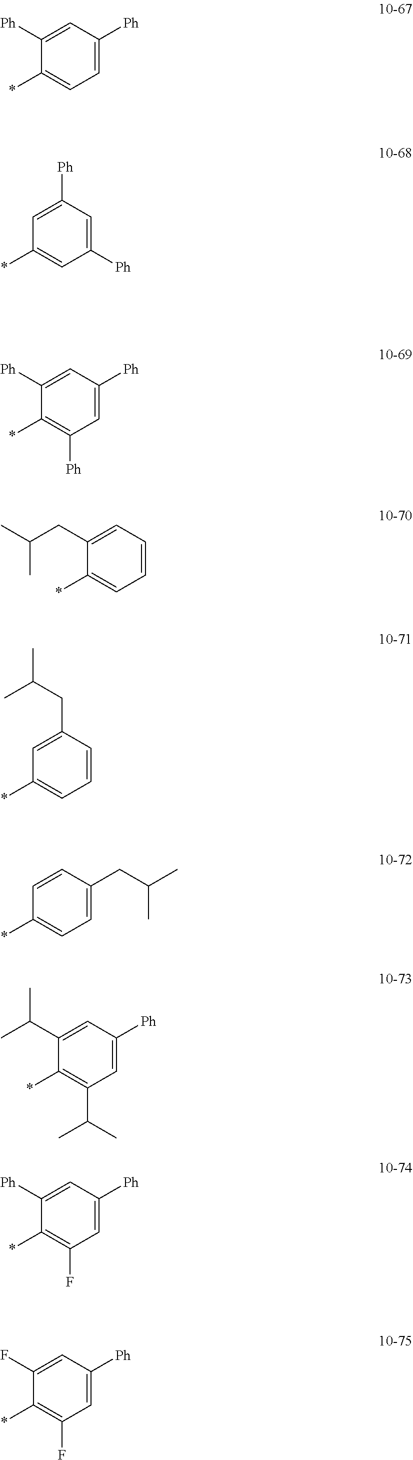 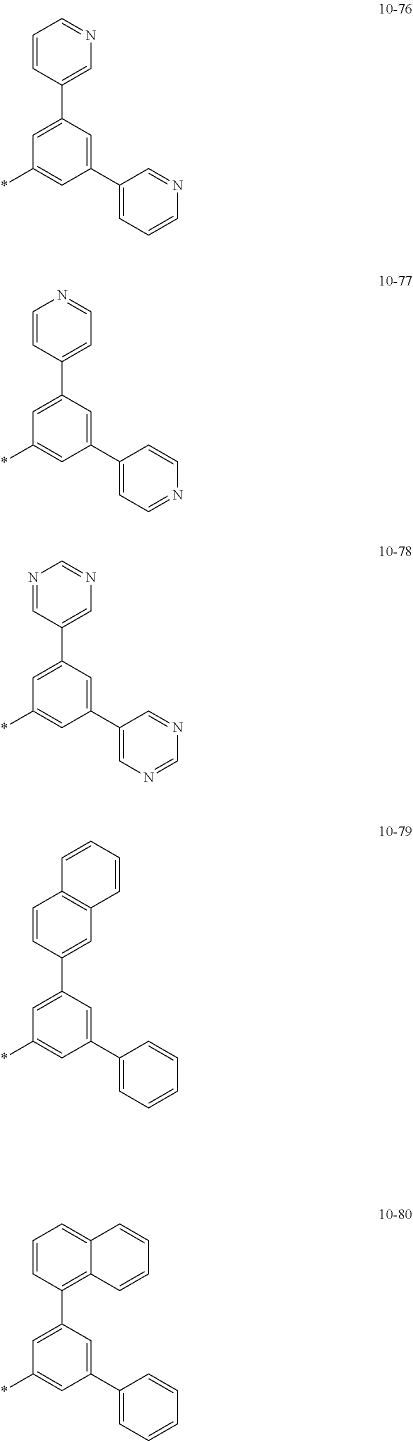 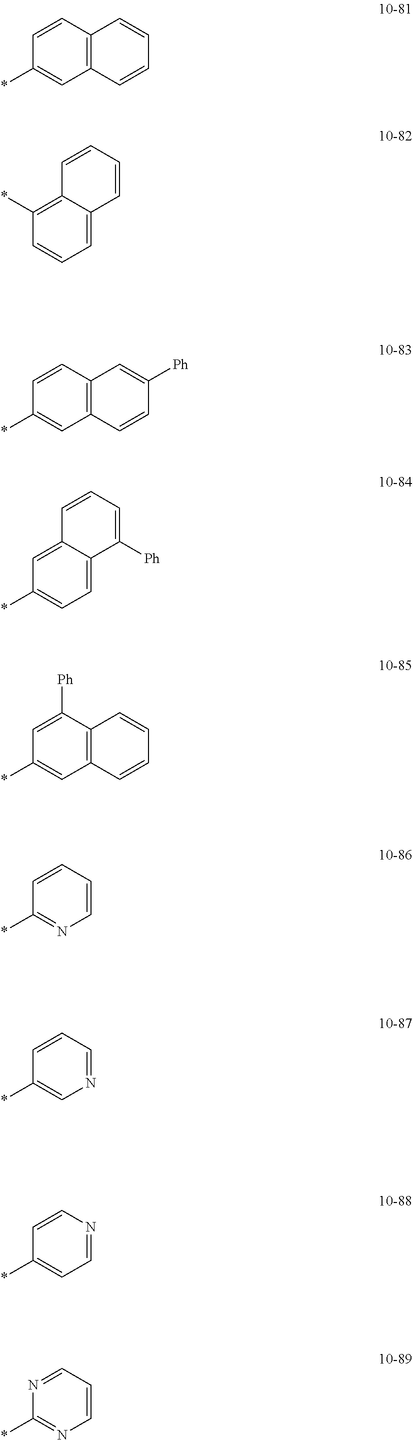 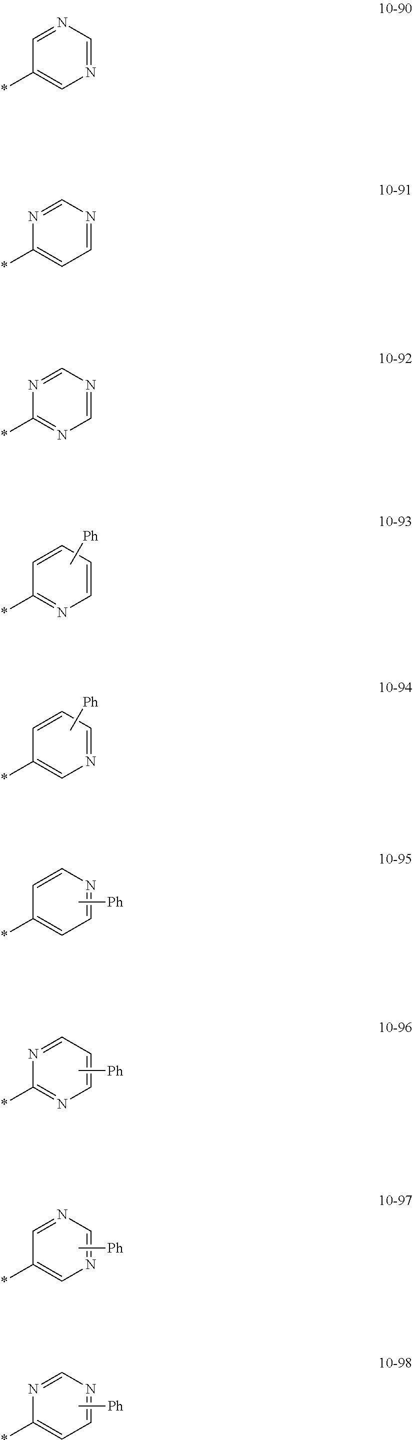 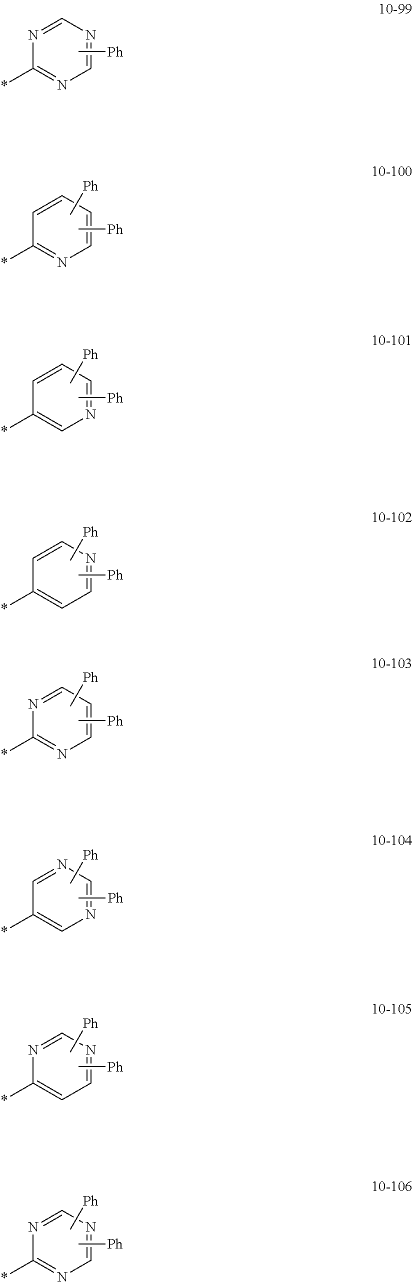 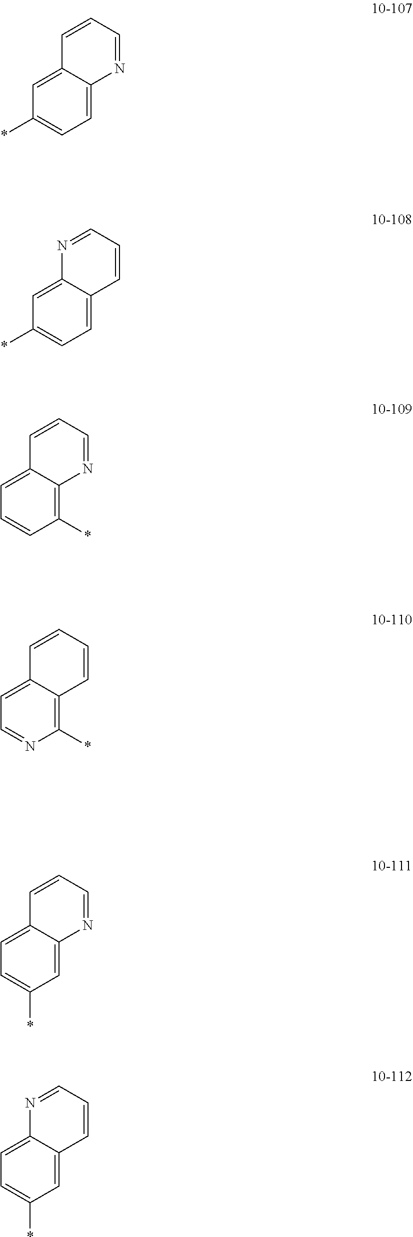 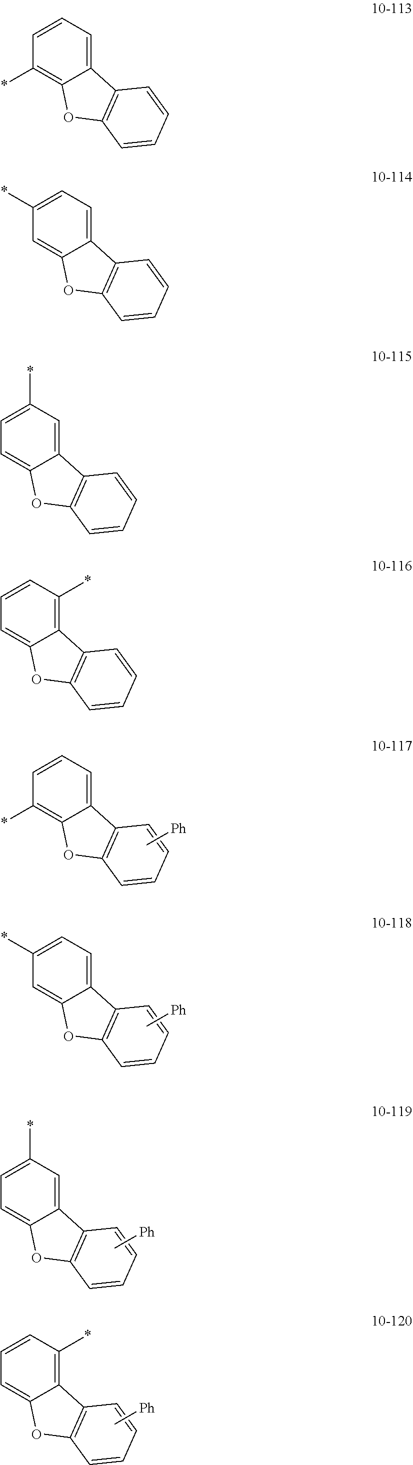 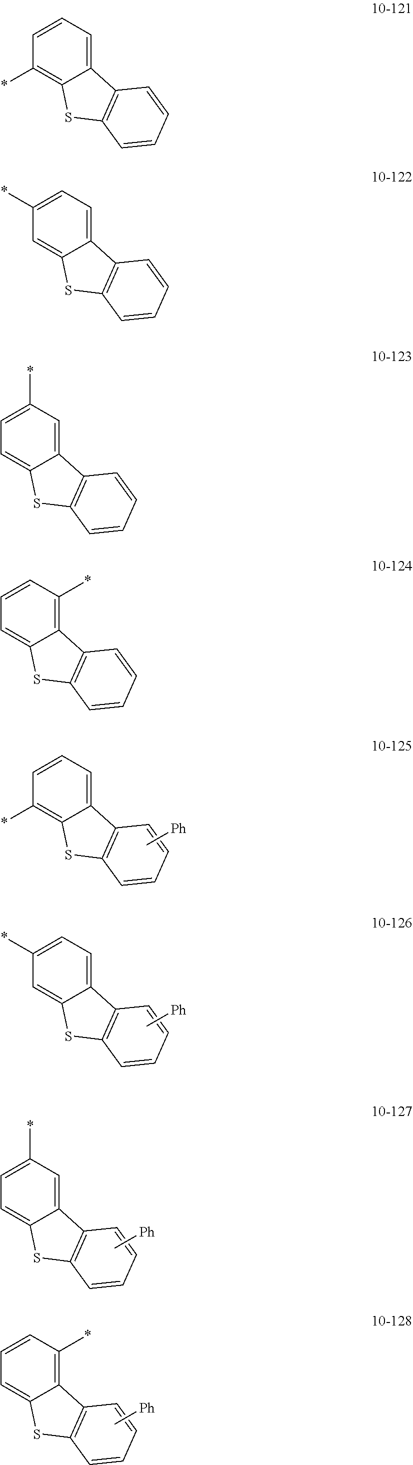 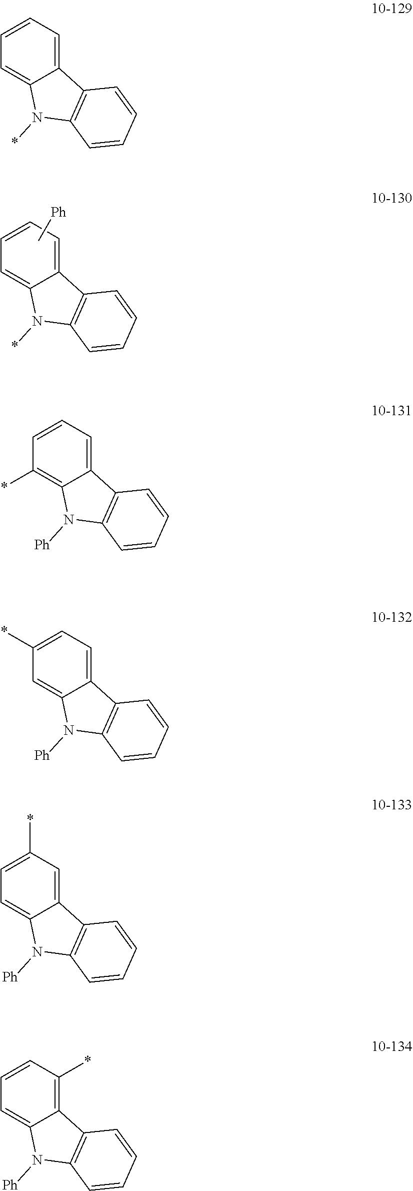 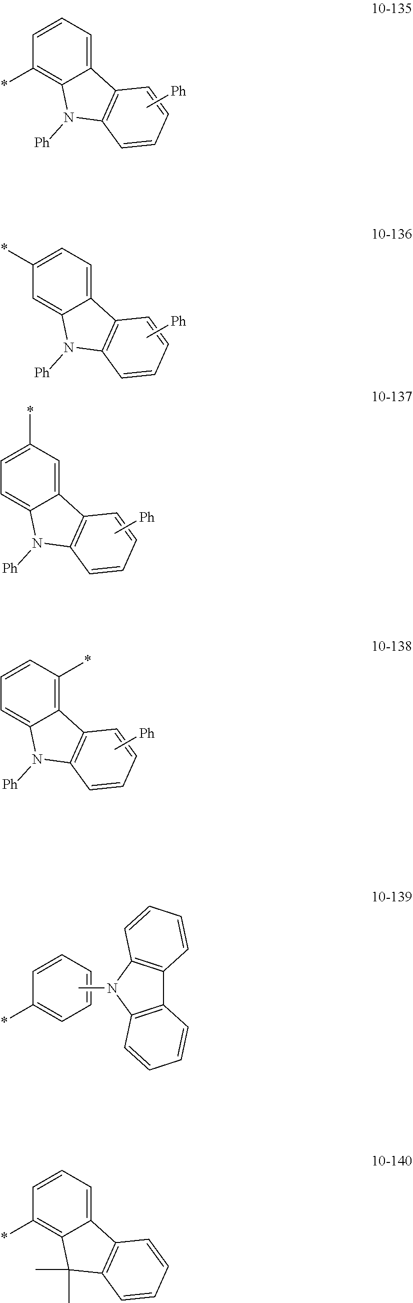 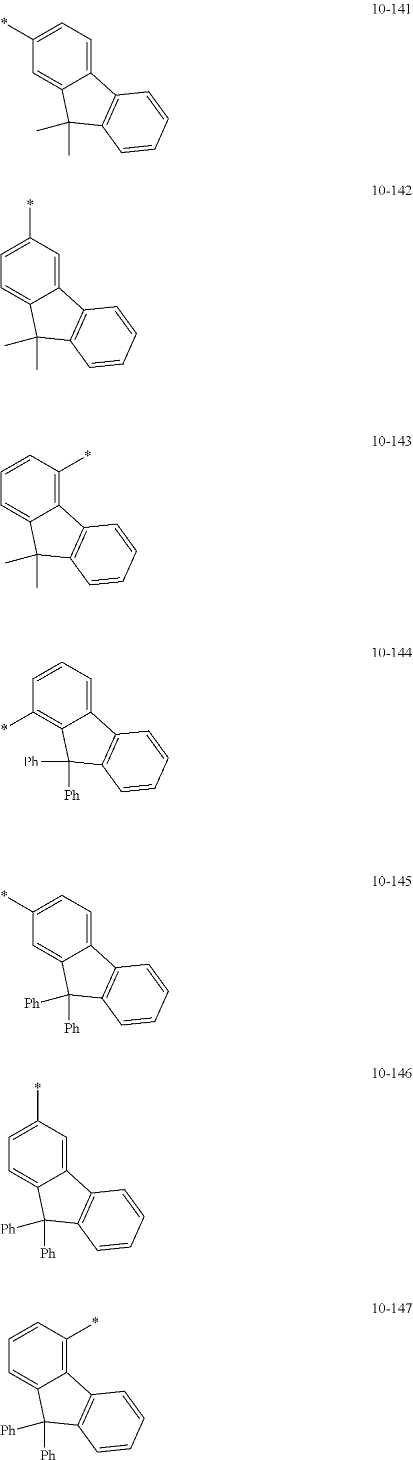 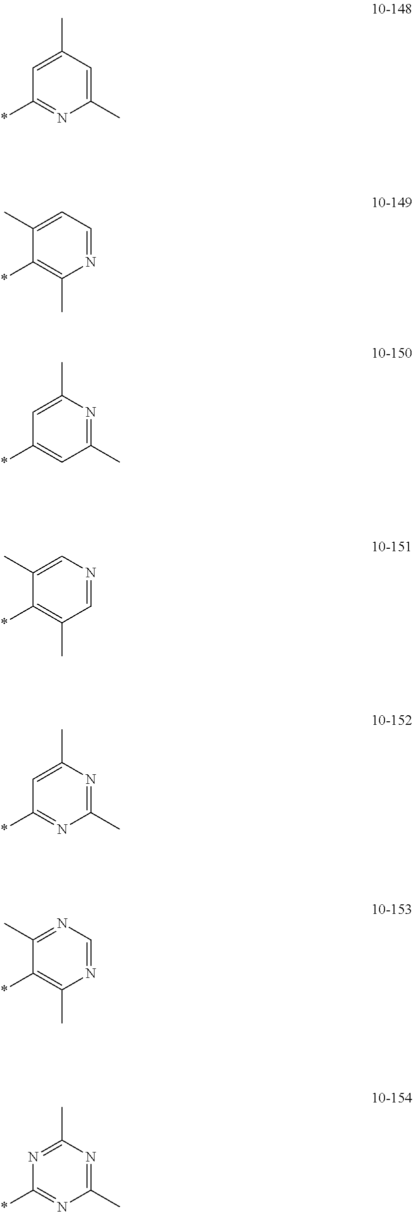 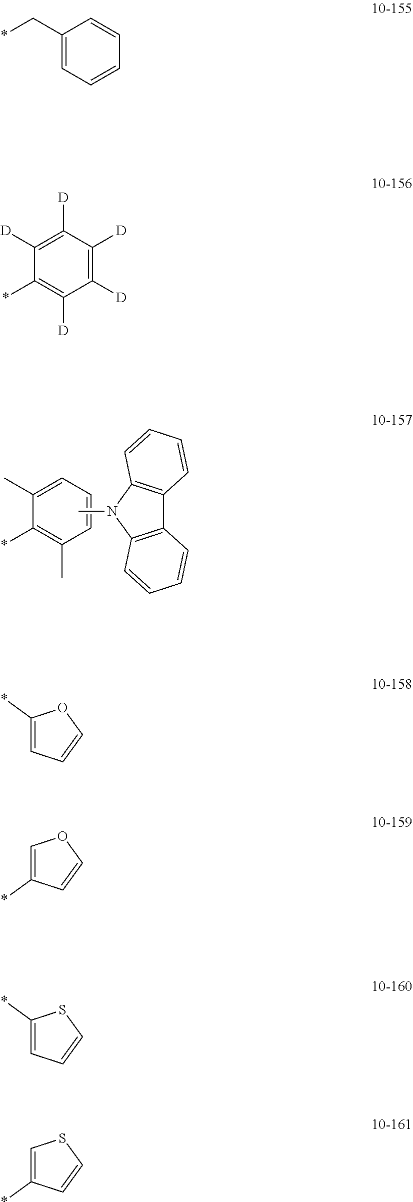 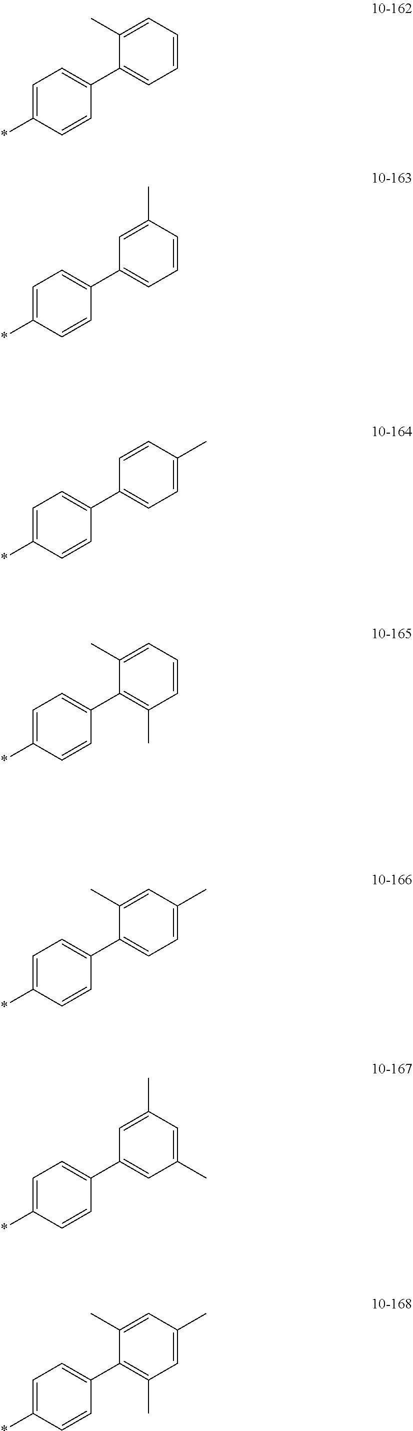 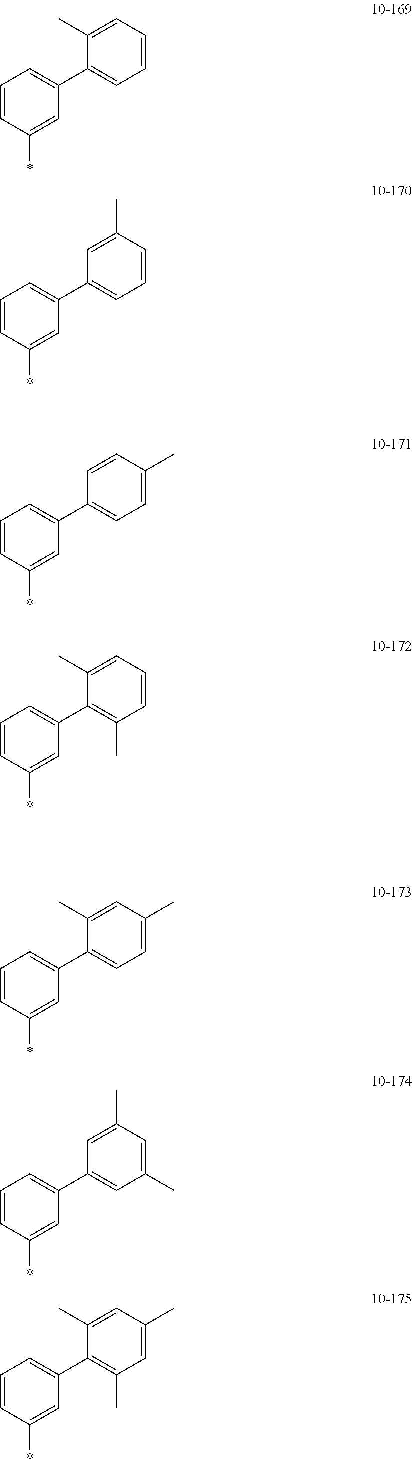 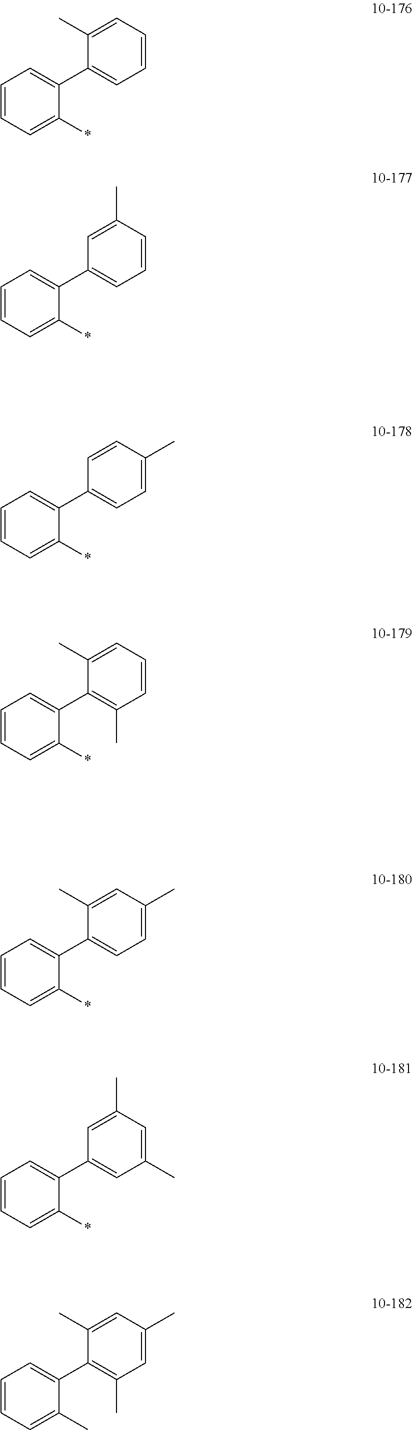 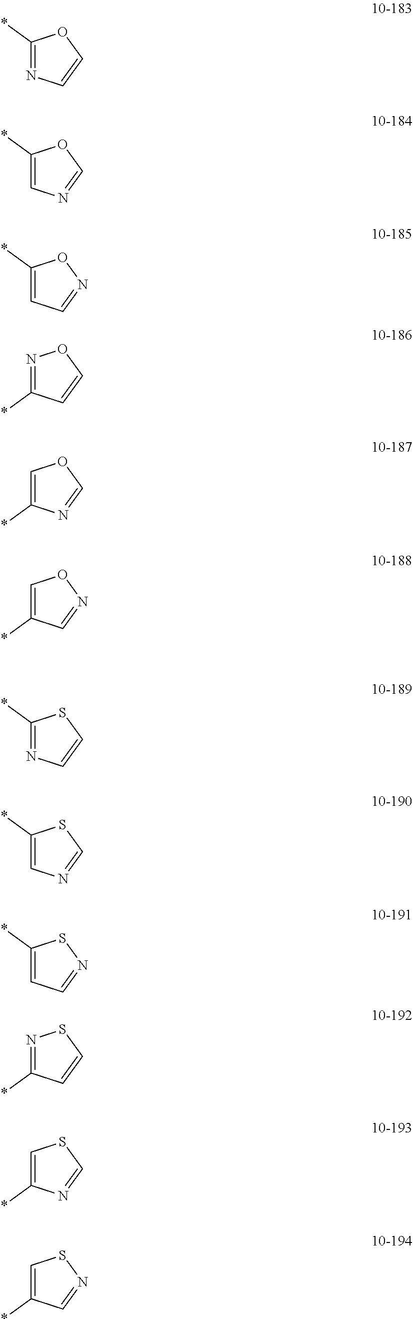 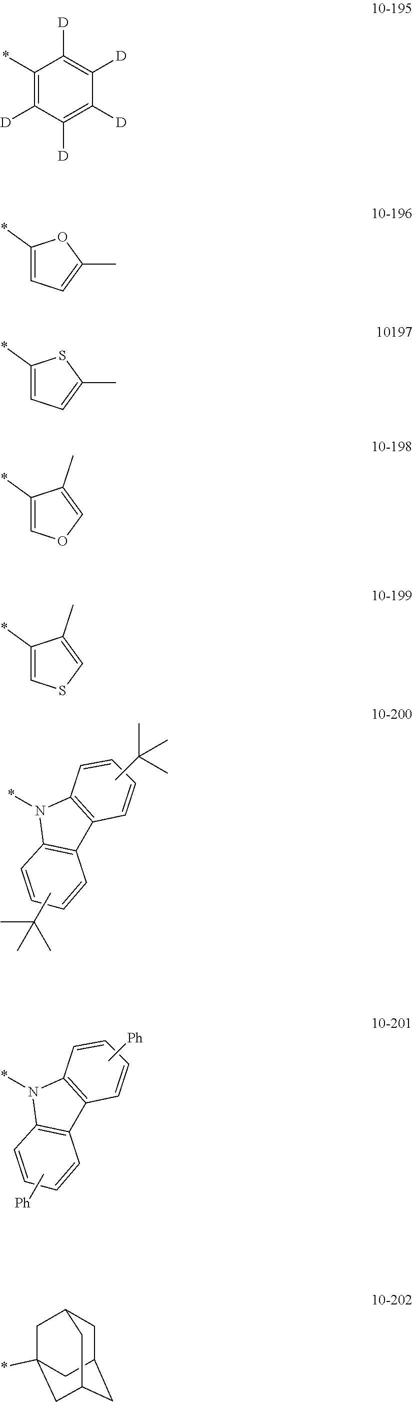 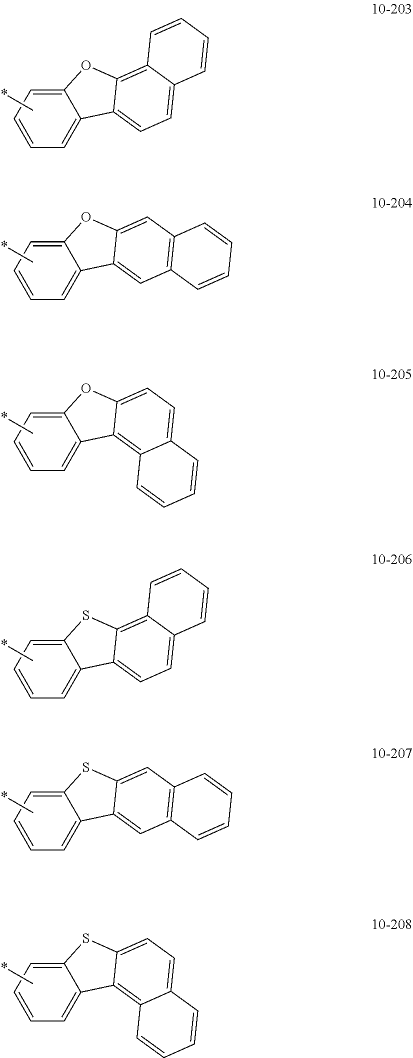
wherein, in Formulae 9-1 to 9-19 and 10-1 to 10-208, * indicates a binding site to a neighboring atom, Ph is a phenyl group, and TMS is a trimethylsilyl group.

7. The heterocyclic compound of claim 1, wherein R₁₀, R₂₀, R₃₀, and R₄₀ are each independently:
hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, or any combination thereof;
a C₁-C₂₀ alkyl group. a C₁-C₂₀ alkoxy group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a pyridinyl group, a pyrimidinyl group, a pyrazinyl group, a pyridazinyl group, a triazinyl group, or any combination thereof;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a thazinyl group, a dibenzofuranyl group, a dibenzothiophenyl group, an imidazopyrimidinyl group, an imidazopyridinyl group, or any combination thereof; or
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyrimidinyl group, an imidazopyridinyl group, or any combination thereof, each substituted with at least one deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, or any combination thereof.

8. The heterocyclic compound of claim 1, wherein R₁₀, R₂₀, R₃₀, and R₄₀ are each independently:
hydrogen, deuterium, a cyano group, a C₁-C₂₀ alkyl group, a C₁-C₂₀ alkoxy group, or any combination thereof;
a C₁-C₂₀ alkyl group, a C₁-C₂₀ alkoxy group, or any combination thereof, each substituted with at least one deuterium, a cyano group, a phenyl group, a biphenyl group, a terphenyl group, a naphthyl group, or any combination thereof;
a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a carbazolyl group, a dibenzofuranyl group, a dibenzothiophenyl group, or any combination thereof; or
a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a carbazolyl group, a dibenzofuranyl group, a dibenzothiophenyl group, or any combination thereof, each substituted with at least one deuterium, a cyano group, a C₁-C₂₀ alkyl group, a C₁-C₂₀ alkoxy group, a phenyl group, a biphenyl group, a terphenyl group, a naphthyl group, or any combination thereof.

9. The heterocyclic compound of claim 1, wherein the heterocyclic compound represented by Formula 1 is a compound represented by one of Formulas 10-1 to 10-6: 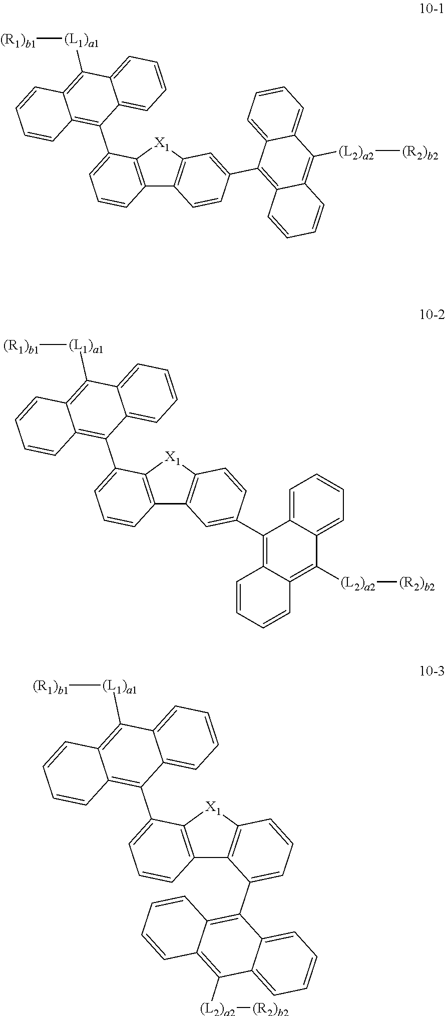 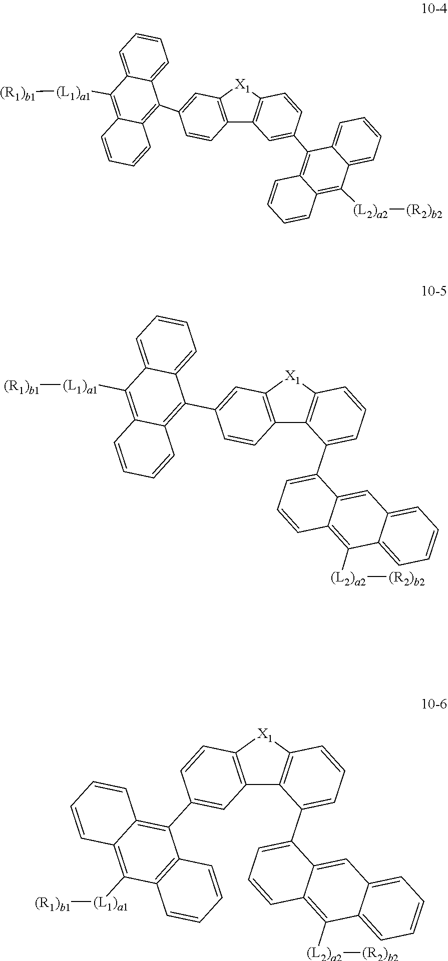
wherein, in Formulae 10-1 to 10-6,
X₁, L₁, L₂, a1, a2, R₁, R₂, b1, and b2 are the same as described in connection with claim 1.

10. The heterocyclic compound of claim 1, wherein the heterocyclic compound represented by Formula 1 satisfies Equation 1:
E(T1)<E(S1)<2×E(T1) Equation 1
wherein, in Equation 1,
E(T1) indicates a lowest excitation triplet energy level of the heterocyclic compound and E(S1) indicates a lowest excitation singlet energy level of the heterocyclic compound.

11. The heterocyclic compound of claim 1, wherein the heterocyclic compound represented by Formula 1 satisfies Equation 2:
[2×E(T1)]−E(S1)<0.5 eV Equation 2
wherein, in Equation 2,
E(T1) indicates a lowest excitation triplet energy level of the heterocyclic compound and E(S1) indicates a lowest excitation singlet energy level of the heterocyclic compound.

12. The heterocyclic compound of claim 1, wherein the heterocyclic compound represented by Formula 1 is one of the following compounds:

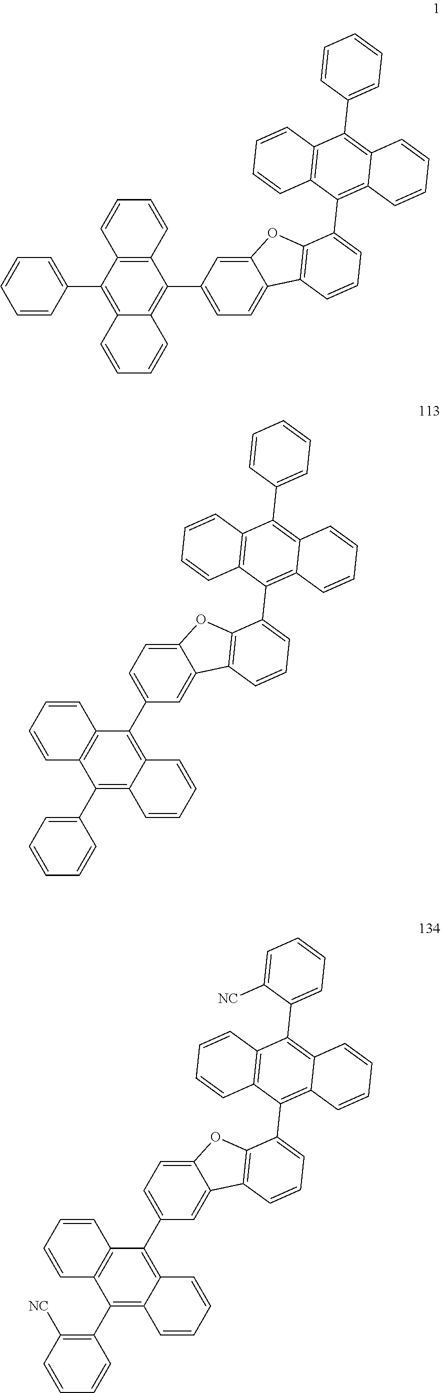 [SMILES: c1ccc(-c2c3ccccc3c(-c3ccc4c(c3)oc3c(-c5c6ccccc6c(-c6ccccc6)c6ccccc56)cccc34)c3ccccc23)cc1 ; c1ccc(-c2c3ccccc3c(-c3ccc4oc5c(-c6c7ccccc7c(-c7ccccc7)c7ccccc67)cccc5c4c3)c3ccccc23)cc1 ; N#Cc1ccccc1-c1c2ccccc2c(-c2ccc3oc4c(-c5c6ccccc6c(-c6ccccc6C#N)c6ccccc56)cccc4c3c2)c2ccccc12]

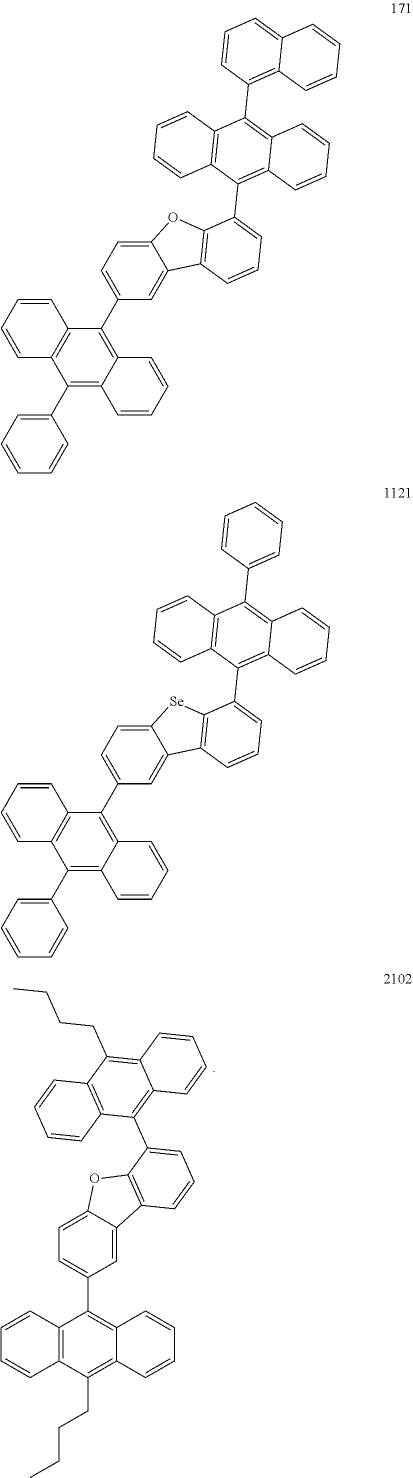

13. An organic light-emitting device comprising:
a first electrode;
a second electrode; and
an organic layer located between the first electrode and the second electrode and comprising an emission layer,
wherein the organic layer comprises at least one of the heterocyclic compound represented by Formula 1 of claim 1.

14. The organic light-emitting device of claim 13, wherein
the first electrode is an anode and the second electrode is a cathode,
the organic layer comprises a hole transport region between the first electrode and the emission layer and an electron transport region between the emission layer and the second electrode,
wherein the hole transport region comprises at least one of a hole injection layer, a hole transport layer, an electron blocking layer or any combination thereof, and
the electron transport region comprises at least one of a hole blocking layer, an electron transport layer, an electron injection layer, or any combination thereof.

15. The organic light-emitting device of claim 13, wherein the heterocyclic compound represented by Formula 1 is included in the emission layer.

16. The organic light-emitting device of claim 15, wherein the emission layer comprises a host and a dopant,
wherein the host comprises the heterocyclic compound represented by Formula 1, and an amount of the host is greater than that of the dopant.

17. The organic light-emitting device of claim 16, wherein the dopant is a fluorescent dopant.

18. The organic light-emitting device of claim 15, wherein the emission layer emits light having a maximum emission wavelength of about 410 nm to about 490 nm.

19. The organic light-emitting device of claim 14, wherein the heterocyclic compound represented by Formula 1 is included in the hole transport region.

20. The organic light-emitting device of claim 14, wherein the heterocyclic compound represented by Formula 1 is included in the electron transport region.
